(19) United States
(12) Patent Application Publication
(10) Pub. No.: US 2025/0263681 A1
(43) Pub. Date: Aug. 21, 2025

(54) LIPID NANOPARTICLES FOR GENE EDITING SYSTEMS

(71) Applicant: ORTHOBIO THERAPEUTICS, INC., Vail, CO (US)

(72) Inventors: Peter J. Millet, Edwards, CO (US); Matthew Allen, Swaffham Bulbeck, Cambs (GB); George Gentsch, Cambridge (GB)

(21) Appl. No.: 18/856,005

(22) PCT Filed: Apr. 12, 2023
(86) PCT No.: PCT/US2023/065691

§ 371 Date: Oct. 10, 2024

Related U.S. Application Data
(60) Provisional application No. 63/362,858, filed on Apr. 12, 2022.
(60) Provisional application No. 63/334,476, filed on Apr. 25, 2022.
(60) Provisional application No. 63/342,471, filed on May 16, 2022.
(60) Provisional application No. 63/495,461, filed on Apr. 11, 2023.

(51) Int. Cl. C12N 9/22; C12N 15/11

(52) U.S. Cl. CPC C12N 9/226; C12N 15/111; C12N 2310/20

(57) ABSTRACT

The present disclosure provides compositions and methods for treating and preventing localized nociception, inflammation, or morphological changes associated with joint disease or illness, back or spine conditions or disorders, and musculoskeletal diseases or dysfunction with an LNP-encapsulated CRISPR/Cas9 gene editing system.

### CROSS REFERENCE TO RELATED APPLICATIONS

This application claims priority to U.S. Provisional Patent Application No. 63/334,476, filed Apr. 25, 2022, U.S. Provisional Patent Application No. 63/362,858, filed Apr. 12, 2022, U.S. Provisional Patent Application No. 63/342,471, filed May 16, 2022, and U.S. Provisional Patent Application No. 63/495,461, filed Apr. 11, 2023, the contents of which are hereby incorporated by reference herein, in their entireties, for all purposes.

### BACKGROUND OF THE DISCLOSURE

### Joint Disorders

Treatment of osteoarthritis, degenerative joint disease, and other joint dysfunction is complex and there are few long term options for either symptomatic relief or restoring joint function. Osteoarthritis (OA) is the leading cause of disability due to pain. Neogi, Osteoarthritis Cartilage 2013; 21:1145-53. All mammal species are affected: working animals, domestic pets, and their owners all suffer OA-related discomfort, pain, and disability, depending on the degree of disease progression.

OA is a complex disease characterized by a progressive course of disability. Systemic inflammation is associated with OA and with OA disease progression. Inflammation is driven by increased levels of pro-inflammatory cytokines. New methods and compositions to treat this disease are acutely needed. Disclosed herein are compositions and methods useful for treating OA as well as other inflammatory joint disorders.

### Back and Spine Disorders

Back or spine conditions or disorders, including low back pain, and pain or inflammation associated with discogenic disorders e.g., degenerative disc disease (DDD) or internal disc disruption (IDD), is a major cause of morbidity and disability worldwide for which few long-term options for amelioration currently exist. Andersson GB. Epidemiological features of chronic low-back pain. Lancet. 1999; 354:581-585. Presently available treatments include surgical or less invasive options that often fail to offer long-term palliation. Ju, et al. Global Spine Journal (2020): 2192568220963058. All vertebrate species are affected by back or spine conditions or disorders, including working animals, domestic pets, and their owners. All suffer from the associated discomfort, pain, and disability, depending on the degree of disease progression.

Back or spine conditions or disorders, such as low back pain, are complex diseases characterized by a multitude of inputs contributing to a progressive course of disability. Among these contributors are morphological irregularities (e.g., disc disruptions), inflammation, and changes in the localized cellular environment (e.g., vascularization and/or innervation). Peng, Bao-Gan. World Journal of Orthopedics 4.2 (2013): 42. Each contributing factor is driven by differential expression of various gene products, including at least pro-inflammatory cytokines, growth factors and other effector biomolecules. New methods and compositions to treat this disease are acutely needed.

### BRIEF SUMMARY OF THE DISCLOSURE

Provided herein are compositions and methods for treating synovial joint dysfunction, are described herein. In addition, compositions, and methods for treating or preventing localized nociception, inflammation, or morphological changes associated with back or spine conditions or disorders, are disclosed herein. Further, compositions and methods for the treating or preventing musculoskeletal disease and dysfunction, including fibrosis and/or scarring in, for example, post-operative subjects are herein described. Additionally, methods for gene-editing cells, including, but not limited to synovial cells and/or synoviocytes, chondrocytes, synovial macrophages, and synovial fibroblasts, and uses of gene-edited synovial cells and/or synoviocytes, chondrocytes, synovial macrophages, and synovial fibroblasts, in the treatment of diseases such as osteoarthritis are disclosed herein.

### BRIEF DESCRIPTION OF THE DRAWINGS

The presently disclosed embodiments will be further explained with reference to the attached drawings. The drawings shown are not necessarily to scale, with emphasis instead generally being placed upon illustrating the principles of the presently disclosed embodiments.

FIG. 1 illustrates SEQ ID NOs: 1-48, the crRNA sequences generated by the bioinformatic methods herein described that target human ADAM17 to modify and/or ablate expression of its encoded products. Additional information includes the chromosomal genomic coordinates (assembly hg38) of the edit site and a score, summarizing several predicted performance metrics.

FIG. 2 illustrates SEQ ID NOs: 49-96, the crRNA sequences generated by the bioinformatic methods herein described that target human ADAMTS1 to modify and/or ablate expression of its encoded products. Additional information includes the chromosomal genomic coordinates (assembly hg38) of the edit site and a score, summarizing several predicted performance metrics.

FIG. 3 illustrates SEQ ID NOs: 97-144, the crRNA sequences generated by the bioinformatic methods herein described that target human ADAMTS5 to modify and/or ablate expression of its encoded products. Additional information includes the chromosomal genomic coordinates (assembly hg38) of the edit site and a score, summarizing several predicted performance metrics.

FIG. 4 illustrates SEQ ID NOs: 145-192, the crRNA sequences generated by the bioinformatic methods herein described that target human ADM to modify and/or ablate expression of its encoded products. Additional information includes the chromosomal genomic coordinates (assembly hg38) of the edit site and a score, summarizing several predicted performance metrics.

FIG. 5 illustrates SEQ ID NOs: 193-240, the crRNA sequences generated by the bioinformatic methods herein described that target human ATP1A1 to modify and/or ablate expression of its encoded products. Additional information includes the chromosomal genomic coordinates (assembly hg38) of the edit site and a score, summarizing several predicted performance metrics.

FIG. 6 illustrates SEQ ID NOs: 241-281, the crRNA sequences generated by the bioinformatic methods herein described that target human BDNF to modify and/or ablate expression of its encoded products. Additional information includes the chromosomal genomic coordinates (assembly hg38) of the edit site and a score, summarizing several predicted performance metrics.

FIG. 7 illustrates SEQ ID NOs: 282-301, the crRNA sequences generated by the bioinformatic methods herein described that target human CALCA to modify and/or ablate expression of its encoded products. Additional information includes the chromosomal genomic coordinates (assembly hg38) of the edit site and a score, summarizing several predicted performance metrics.

FIG. 8 illustrates SEQ ID NOs: 302-318, the crRNA sequences generated by the bioinformatic methods herein described that target human CALCB to modify and/or ablate expression of its encoded products. Additional information includes the chromosomal genomic coordinates (assembly hg38) of the edit site and a score, summarizing several predicted performance metrics.

FIG. 9 illustrates SEQ ID NOs: 319-340, the crRNA sequences generated by the bioinformatic methods herein described that target human CALCRL to modify and/or ablate expression of its encoded products. Additional information includes the chromosomal genomic coordinates (assembly hg38) of the edit site and a score, summarizing several predicted performance metrics.

FIG. 10 illustrates SEQ ID NOs: 341-357, the crRNA sequences generated by the bioinformatic methods herein described that target human CCL2 to modify and/or ablate expression of its encoded products. Additional information includes the chromosomal genomic coordinates (assembly hg38) of the edit site and a score, summarizing several predicted performance metrics.

FIG. 11 illustrates SEQ ID NOs: 358-374, the crRNA sequences generated by the bioinformatic methods herein described that target human CCL3 to modify and/or ablate expression of its encoded products. Additional information includes the chromosomal genomic coordinates (assembly hg38) of the edit site and a score, summarizing several predicted performance metrics.

FIG. 12 illustrates SEQ ID NOs: 375-391, the crRNA sequences generated by the bioinformatic methods herein described that target human CCL5 to modify and/or ablate expression of its encoded products. Additional information includes the chromosomal genomic coordinates (assembly hg38) of the edit site and a score, summarizing several predicted performance metrics.

FIG. 13 illustrates SEQ ID NOs: 392-408, the crRNA sequences generated by the bioinformatic methods herein described that target human CCL7 to modify and/or ablate expression of its encoded products. Additional information includes the chromosomal genomic coordinates (assembly hg38) of the edit site and a score, summarizing several predicted performance metrics.

FIG. 14 illustrates SEQ ID NOs: 409-425, the crRNA sequences generated by the bioinformatic methods herein described that target human CCL20 to modify and/or ablate expression of its encoded products. Additional information includes the chromosomal genomic coordinates (assembly hg38) of the edit site and a score, summarizing several predicted performance metrics.

FIG. 15 illustrates SEQ ID NOs: 426-473, the crRNA sequences generated by the bioinformatic methods herein described that target human CCN2 to modify and/or ablate expression of its encoded products. Additional information includes the chromosomal genomic coordinates (assembly hg38) of the edit site and a score, summarizing several predicted performance metrics.

FIG. 16 illustrates SEQ ID NOs: 474-517, the crRNA sequences generated by the bioinformatic methods herein described that target human CCR7 to modify and/or ablate expression of its encoded products. Additional information includes the chromosomal genomic coordinates (assembly hg38) of the edit site and a score, summarizing several predicted performance metrics.

FIG. 17 illustrates SEQ ID NOs: 518-534, the crRNA sequences generated by the bioinformatic methods herein described that target human CRCP to modify and/or ablate expression of its encoded products. Additional information includes the chromosomal genomic coordinates (assembly hg38) of the edit site and a score, summarizing several predicted performance metrics.

FIG. 18 illustrates SEQ ID NOs: 535-551, the crRNA sequences generated by the bioinformatic methods herein described that target human CXCL1 to modify and/or ablate expression of its encoded products. Additional information includes the chromosomal genomic coordinates (assembly hg38) of the edit site and a score, summarizing several predicted performance metrics.

FIG. 19 illustrates SEQ ID NOs: 552-568, the crRNA sequences generated by the bioinformatic methods herein described that target human CXCL2 to modify and/or ablate expression of its encoded products. Additional information includes the chromosomal genomic coordinates (assembly hg38) of the edit site and a score, summarizing several predicted performance metrics.

FIG. 20 illustrates SEQ ID NOs: 569-585, the crRNA sequences generated by the bioinformatic methods herein described that target human CXCL3 to modify and/or ablate expression of its encoded products. Additional information includes the chromosomal genomic coordinates (assembly hg38) of the edit site and a score, summarizing several predicted performance metrics.

FIG. 21 illustrates SEQ ID NOs: 586-602, the crRNA sequences generated by the bioinformatic methods herein described that target human CXCL5 to modify and/or ablate expression of its encoded products. Additional information includes the chromosomal genomic coordinates (assembly hg38) of the edit site and a score, summarizing several predicted performance metrics.

FIG. 22 illustrates SEQ ID NOs: 603-619, the crRNA sequences generated by the bioinformatic methods herein described that target human CXCL6 to modify and/or ablate expression of its encoded products. Additional information includes the chromosomal genomic coordinates (assembly hg38) of the edit site and a score, summarizing several predicted performance metrics.

FIG. 23 illustrates SEQ ID NOs: 620-636, the crRNA sequences generated by the bioinformatic methods herein described that target human CXCL8 to modify and/or ablate expression of its encoded products. Additional information includes the chromosomal genomic coordinates (assembly hg38) of the edit site and a score, summarizing several predicted performance metrics.

FIG. 24 illustrates SEQ ID NOs: 637-655, the crRNA sequences generated by the bioinformatic methods herein described that target human CXCR1 to modify and/or ablate expression of its encoded products. Additional information includes the chromosomal genomic coordinates (assembly hg38) of the edit site and a score, summarizing several predicted performance metrics.

FIG. 25 illustrates SEQ ID NOs: 656-672, the crRNA sequences generated by the bioinformatic methods herein described that target human CXCR2 to modify and/or ablate expression of its encoded products. Additional information includes the chromosomal genomic coordinates (assembly hg38) of the edit site and a score, summarizing several predicted performance metrics.

FIG. 26 illustrates SEQ ID NOs: 673-720, the crRNA sequences generated by the bioinformatic methods herein described that target human FGF2 to modify and/or ablate expression of its encoded products. Additional information includes the chromosomal genomic coordinates (assembly hg38) of the edit site and a score, summarizing several predicted performance metrics.

FIG. 27 illustrates SEQ ID NOs: 721-768, the crRNA sequences generated by the bioinformatic methods herein described that target human FGFR1 to modify and/or ablate expression of its encoded products. Additional information includes the chromosomal genomic coordinates (assembly hg38) of the edit site and a score, summarizing several predicted performance metrics.

FIG. 28 illustrates SEQ ID NOs: 769-786, the crRNA sequences generated by the bioinformatic methods herein described that target human IL1A to modify and/or ablate expression of its encoded products. Additional information includes the chromosomal genomic coordinates (assembly hg38) of the edit site and a score, summarizing several predicted performance metrics.

FIG. 29 illustrates SEQ ID NOs: 787-805, the crRNA sequences generated by the bioinformatic methods herein described that target human IL1B to modify and/or ablate expression of its encoded products. Additional information includes the chromosomal genomic coordinates (assembly hg38) of the edit site and a score, summarizing several predicted performance metrics.

FIG. 30 illustrates SEQ ID NOs: 806-839, the crRNA sequences generated by the bioinformatic methods herein described that target human IL1R1 to modify and/or ablate expression of its encoded products. Additional information includes the chromosomal genomic coordinates (assembly hg38) of the edit site and a score, summarizing several predicted performance metrics.

FIG. 31 illustrates SEQ ID NOs: 840-887, the crRNA sequences generated by the bioinformatic methods herein described that target human IL1RAP to modify and/or ablate expression of its encoded products. Additional information includes the chromosomal genomic coordinates (assembly hg38) of the edit site and a score, summarizing several predicted performance metrics.

FIG. 32 illustrates SEQ ID NOs: 888-911, the crRNA sequences generated by the bioinformatic methods herein described that target human IL4 to modify and/or ablate expression of its encoded products. Additional information includes the chromosomal genomic coordinates (assembly hg38) of the edit site and a score, summarizing several predicted performance metrics.

FIG. 33 illustrates SEQ ID NOs: 912-928, the crRNA sequences generated by the bioinformatic methods herein described that target human IL6 to modify and/or ablate expression of its encoded products. Additional information includes the chromosomal genomic coordinates (assembly hg38) of the edit site and a score, summarizing several predicted performance metrics.

FIG. 34 illustrates SEQ ID NOs: 929-963, the crRNA sequences generated by the bioinformatic methods herein described that target human IL6R to modify and/or ablate expression of its encoded products. Additional information includes the chromosomal genomic coordinates (assembly hg38) of the edit site and a score, summarizing several predicted performance metrics.

FIG. 35 illustrates SEQ ID NOs: 964-990, the crRNA sequences generated by the bioinformatic methods herein described that target human IL6ST to modify and/or ablate expression of its encoded products. Additional information includes the chromosomal genomic coordinates (assembly hg38) of the edit site and a score, summarizing several predicted performance metrics.

FIG. 36 illustrates SEQ ID NOs: 991-1007, the crRNA sequences generated by the bioinformatic methods herein described that target human IL10 to modify and/or ablate expression of its encoded products. Additional information includes the chromosomal genomic coordinates (assembly hg38) of the edit site and a score, summarizing several predicted performance metrics.

FIG. 37 illustrates SEQ ID NOs: 1008-1055, the crRNA sequences generated by the bioinformatic methods herein described that target human IL10RA to modify and/or ablate expression of its encoded products. Additional information includes the chromosomal genomic coordinates (assembly hg38) of the edit site and a score, summarizing several predicted performance metrics.

FIG. 38 illustrates SEQ ID NOs: 1056-1082, the crRNA sequences generated by the bioinformatic methods herein described that target human IL10RB to modify and/or ablate expression of its encoded products. Additional information includes the chromosomal genomic coordinates (assembly hg38) of the edit site and a score, summarizing several predicted performance metrics.

FIG. 39 illustrates SEQ ID NOs: 1083-1104, the crRNA sequences generated by the bioinformatic methods herein described that target human IL13 to modify and/or ablate expression of its encoded products. Additional information includes the chromosomal genomic coordinates (assembly hg38) of the edit site and a score, summarizing several predicted performance metrics.

FIG. 40 illustrates SEQ ID NOs: 1105-1130, the crRNA sequences generated by the bioinformatic methods herein described that target human IL13RA1 to modify and/or ablate expression of its encoded products. Additional information includes the chromosomal genomic coordinates (assembly hg38) of the edit site and a score, summarizing several predicted performance metrics.

FIG. 41 illustrates SEQ ID NOs: 1131-1147, the crRNA sequences generated by the bioinformatic methods herein described that target human IL13RA2 to modify and/or ablate expression of its encoded products. Additional information includes the chromosomal genomic coordinates (assembly hg38) of the edit site and a score, summarizing several predicted performance metrics.

FIG. 42 illustrates SEQ ID NOs: 1148-1173, the crRNA sequences generated by the bioinformatic methods herein described that target human IL17A to modify and/or ablate expression of its encoded products. Additional information includes the chromosomal genomic coordinates (assembly hg38) of the edit site and a score, summarizing several predicted performance metrics.

FIG. 43 illustrates SEQ ID NOs: 1174-1221, the crRNA sequences generated by the bioinformatic methods herein described that target human IL17RA to modify and/or ablate expression of its encoded products. Additional information includes the chromosomal genomic coordinates (assembly hg38) of the edit site and a score, summarizing several predicted performance metrics.

FIG. 44 illustrates SEQ ID NOs: 1222-1238, the crRNA sequences generated by the bioinformatic methods herein described that target human IL18 to modify and/or ablate expression of its encoded products. Additional information includes the chromosomal genomic coordinates (assembly hg38) of the edit site and a score, summarizing several predicted performance metrics.

FIG. 45 illustrates SEQ ID NOs: 1239-1262, the crRNA sequences generated by the bioinformatic methods herein described that target human IL18R1 to modify and/or ablate expression of its encoded products. Additional information includes the chromosomal genomic coordinates (assembly hg38) of the edit site and a score, summarizing several predicted performance metrics.

FIG. 46 illustrates SEQ ID NOs: 1263-1310, the crRNA sequences generated by the bioinformatic methods herein described that target human IL18RAP to modify and/or ablate expression of its encoded products. Additional information includes the chromosomal genomic coordinates (assembly hg38) of the edit site and a score, summarizing several predicted performance metrics.

FIG. 47 illustrates SEQ ID NOs: 1311-1343, the crRNA sequences generated by the bioinformatic methods herein described that target human MMP1 to modify and/or ablate expression of its encoded products. Additional information includes the chromosomal genomic coordinates (assembly hg38) of the edit site and a score, summarizing several predicted performance metrics.

FIG. 48 illustrates SEQ ID NOs: 1344-1391, the crRNA sequences generated by the bioinformatic methods herein described that target human MMP2 to modify and/or ablate expression of its encoded products. Additional information includes the chromosomal genomic coordinates (assembly hg38) of the edit site and a score, summarizing several predicted performance metrics.

FIG. 49 illustrates SEQ ID NOs: 1392-1417, the crRNA sequences generated by the bioinformatic methods herein described that target human MMP3 to modify and/or ablate expression of its encoded products. Additional information includes the chromosomal genomic coordinates (assembly hg38) of the edit site and a score, summarizing several predicted performance metrics.

FIG. 50 illustrates SEQ ID NOs: 1418-1436, the crRNA sequences generated by the bioinformatic methods herein described that target human MMP7 to modify and/or ablate expression of its encoded products. Additional information includes the chromosomal genomic coordinates (assembly hg38) of the edit site and a score, summarizing several predicted performance metrics.

FIG. 51 illustrates SEQ ID NOs: 1437-1474, the crRNA sequences generated by the bioinformatic methods herein described that target human MMP8 to modify and/or ablate expression of its encoded products. Additional information includes the chromosomal genomic coordinates (assembly hg38) of the edit site and a score, summarizing several predicted performance metrics.

FIG. 52 illustrates SEQ ID NOs: 1475-1497, the crRNA sequences generated by the bioinformatic methods herein described that target human MMP10 to modify and/or ablate expression of its encoded products. Additional information includes the chromosomal genomic coordinates (assembly hg38) of the edit site and a score, summarizing several predicted performance metrics.

FIG. 53 illustrates SEQ ID NOs: 1498-1541, the crRNA sequences generated by the bioinformatic methods herein described that target human MMP12 to modify and/or ablate expression of its encoded products. Additional information includes the chromosomal genomic coordinates (assembly hg38) of the edit site and a score, summarizing several predicted performance metrics.

FIG. 54 illustrates SEQ ID NOs: 1542-1568, the crRNA sequences generated by the bioinformatic methods herein described that target human MMP13 to modify and/or ablate expression of its encoded products. Additional information includes the chromosomal genomic coordinates (assembly hg38) of the edit site and a score, summarizing several predicted performance metrics.

FIG. 55 illustrates SEQ ID NOs: 1569-1585, the crRNA sequences generated by the bioinformatic methods herein described that target human MRGPRX2 to modify and/or ablate expression of its encoded products. Additional information includes the chromosomal genomic coordinates (assembly hg38) of the edit site and a score, summarizing several predicted performance metrics.

FIG. 56 illustrates SEQ ID NOs: 1586-1628, the crRNA sequences generated by the bioinformatic methods herein described that target human NGF to modify and/or ablate expression of its encoded products. Additional information includes the chromosomal genomic coordinates (assembly hg38) of the edit site and a score, summarizing several predicted performance metrics.

FIG. 57 illustrates SEQ ID NOs: 1629-1676, the crRNA sequences generated by the bioinformatic methods herein described that target human NGFR to modify and/or ablate expression of its encoded products. Additional information includes the chromosomal genomic coordinates (assembly hg38) of the edit site and a score, summarizing several predicted performance metrics.

FIG. 58 illustrates SEQ ID NOs: 1677-1724, the crRNA sequences generated by the bioinformatic methods herein described that target human NTF3 to modify and/or ablate expression of its encoded products. Additional information includes the chromosomal genomic coordinates (assembly hg38) of the edit site and a score, summarizing several predicted performance metrics.

FIG. 59 illustrates SEQ ID NOs: 1725-1746, the crRNA sequences generated by the bioinformatic methods herein described that target human NTF4 to modify and/or ablate expression of its encoded products. Additional information includes the chromosomal genomic coordinates (assembly hg38) of the edit site and a score, summarizing several predicted performance metrics.

FIG. 60 illustrates SEQ ID NOs: 1747-1794, the crRNA sequences generated by the bioinformatic methods herein described that target human NTRK1 to modify and/or ablate expression of its encoded products. Additional information includes the chromosomal genomic coordinates (assembly hg38) of the edit site and a score, summarizing several predicted performance metrics.

FIG. 61 illustrates SEQ ID NOs: 1795-1842, the crRNA sequences generated by the bioinformatic methods herein described that target human NTRK2 to modify and/or ablate expression of its encoded products. Additional information includes the chromosomal genomic coordinates (assembly hg38) of the edit site and a score, summarizing several predicted performance metrics.

FIG. 62 illustrates SEQ ID NOs: 1843-1859, the crRNA sequences generated by the bioinformatic methods herein described that target human RAMP1 to modify and/or ablate expression of its encoded products. Additional information includes the chromosomal genomic coordinates (assembly hg38) of the edit site and a score, summarizing several predicted performance metrics.

FIG. 63 illustrates SEQ ID NOs: 1860-1907, the crRNA sequences generated by the bioinformatic methods herein described that target human SCN1A to modify and/or ablate expression of its encoded products. Additional information includes the chromosomal genomic coordinates (assembly hg38) of the edit site and a score, summarizing several predicted performance metrics.

FIG. 64 illustrates SEQ ID NOs: 1908-1955, the crRNA sequences generated by the bioinformatic methods herein described that target human SCN2A to modify and/or ablate expression of its encoded products. Additional information includes the chromosomal genomic coordinates (assembly hg38) of the edit site and a score, summarizing several predicted performance metrics.

FIG. 65 illustrates SEQ ID NOs: 1956-2003, the crRNA sequences generated by the bioinformatic methods herein described that target human SCN3A to modify and/or ablate expression of its encoded products. Additional information includes the chromosomal genomic coordinates (assembly hg38) of the edit site and a score, summarizing several predicted performance metrics.

FIG. 66 illustrates SEQ ID NOs: 2004-2051, the crRNA sequences generated by the bioinformatic methods herein described that target human SCN4A to modify and/or ablate expression of its encoded products. Additional information includes the chromosomal genomic coordinates (assembly hg38) of the edit site and a score, summarizing several predicted performance metrics.

FIG. 67 illustrates SEQ ID NOs: 2052-2099, the crRNA sequences generated by the bioinformatic methods herein described that target human SCN5A to modify and/or ablate expression of its encoded products. Additional information includes the chromosomal genomic coordinates (assembly hg38) of the edit site and a score, summarizing several predicted performance metrics.

FIG. 68 illustrates SEQ ID NOs: 2100-2147, the crRNA sequences generated by the bioinformatic methods herein described that target human SCN8A to modify and/or ablate expression of its encoded products. Additional information includes the chromosomal genomic coordinates (assembly hg38) of the edit site and a score, summarizing several predicted performance metrics.

FIG. 69 illustrates SEQ ID NOs: 2148-2195, the crRNA sequences generated by the bioinformatic methods herein described that target human SCN9A to modify and/or ablate expression of its encoded products. Additional information includes the chromosomal genomic coordinates (assembly hg38) of the edit site and a score, summarizing several predicted performance metrics.

FIG. 70 illustrates SEQ ID NOs: 2196-2243, the crRNA sequences generated by the bioinformatic methods herein described that target human SCN10A to modify and/or ablate expression of its encoded products. Additional information includes the chromosomal genomic coordinates (assembly hg38) of the edit site and a score, summarizing several predicted performance metrics.

FIG. 71 illustrates SEQ ID NOs: 2244-2291, the crRNA sequences generated by the bioinformatic methods herein described that target human SCN11A to modify and/or ablate expression of its encoded products. Additional information includes the chromosomal genomic coordinates (assembly hg38) of the edit site and a score, summarizing several predicted performance metrics.

FIG. 72 illustrates SEQ ID NOs: 2292-2308, the crRNA sequences generated by the bioinformatic methods herein described that target human TAC1 to modify and/or ablate expression of its encoded products. Additional information includes the chromosomal genomic coordinates (assembly hg38) of the edit site and a score, summarizing several predicted performance metrics.

FIG. 73 illustrates SEQ ID NOs: 2309-2325, the crRNA sequences generated by the bioinformatic methods herein described that target human TAC3 to modify and/or ablate expression of its encoded products. Additional information includes the chromosomal genomic coordinates (assembly hg38) of the edit site and a score, summarizing several predicted performance metrics.

FIG. 74 illustrates SEQ ID NOs: 2326-2373, the crRNA sequences generated by the bioinformatic methods herein described that target human TACR1 to modify and/or ablate expression of its encoded products. Additional information includes the chromosomal genomic coordinates (assembly hg38) of the edit site and a score, summarizing several predicted performance metrics.

FIG. 75 illustrates SEQ ID NOs: 2374-2421, the crRNA sequences generated by the bioinformatic methods herein described that target human TACR2 to modify and/or ablate expression of its encoded products. Additional information includes the chromosomal genomic coordinates (assembly hg38) of the edit site and a score, summarizing several predicted performance metrics.

FIG. 76 illustrates SEQ ID NOs: 2422-2469, the crRNA sequences generated by the bioinformatic methods herein described that target human TACR3 to modify and/or ablate expression of its encoded products. Additional information includes the chromosomal genomic coordinates (assembly hg38) of the edit site and a score, summarizing several predicted performance metrics.

FIG. 77 illustrates SEQ ID NOs: 2470-2509, the crRNA sequences generated by the bioinformatic methods herein described that target human TIMP1 to modify and/or ablate expression of its encoded products. Additional information includes the chromosomal genomic coordinates (assembly hg38) of the edit site and a score, summarizing several predicted performance metrics.

FIG. 78 illustrates SEQ ID NOs: 2510-2557, the crRNA sequences generated by the bioinformatic methods herein described that target human TIMP3 to modify and/or ablate expression of its encoded products. Additional information includes the chromosomal genomic coordinates (assembly hg38) of the edit site and a score, summarizing several predicted performance metrics.

FIG. 79 illustrates SEQ ID NOs: 2558-2574, the crRNA sequences generated by the bioinformatic methods herein described that target human TNF to modify and/or ablate expression of its encoded products. Additional information includes the chromosomal genomic coordinates (assembly hg38) of the edit site and a score, summarizing several predicted performance metrics.

FIG. 80 illustrates SEQ ID NOs: 2575-2622, the crRNA sequences generated by the bioinformatic methods herein described that target human TNFRSF1A to modify and/or ablate expression of its encoded products. Additional information includes the chromosomal genomic coordinates (assembly hg38) of the edit site and a score, summarizing several predicted performance metrics.

FIG. 81 illustrates SEQ ID NOs: 2623-2670, the crRNA sequences generated by the bioinformatic methods herein described that target human TNFRSF1B to modify and/or ablate expression of its encoded products. Additional information includes the chromosomal genomic coordinates (assembly hg38) of the edit site and a score, summarizing several predicted performance metrics.

FIG. 82 illustrates SEQ ID NOs: 2671-2718, the crRNA sequences generated by the bioinformatic methods herein described that target human YAP1 to modify and/or ablate expression of its encoded products. Additional information includes the chromosomal genomic coordinates (assembly hg38) of the edit site and a score, summarizing several predicted performance metrics.

FIGS. 83A, 83B, 83C, and 83D collectively illustrate the results of cell-based and in-silico gene editing analysis of crRNA sequences targeting (A) hIL1A, (B) hIL1B, (C) cIL1A, and (D) cIL1B genes. ‘º’ denotes CRISPR cut position within the translation frame of amino acids (AA). ‘*’ denotes optimized score from Doench, Fusi et al. (2016). This score is optimized for 20 bp guides with an NGG PAM. Score spans from 0 to 100. Higher is better. ** Specificity score from Hsu et al. (2013). Score spans from 0 to 100. Higher is better. *** This score is based on experiments in U2OS. A high precision score (>0.4) implies that DNA repair outcomes are uniform and enriched for just a handful of unique genotypes. **** This score is based on experiments in U2OS. A high (>80%) frameshift frequency will tend to knock a protein-coding gene out of frame. The typical genomic frameshift frequency is above 66% because 1-bp insertions and 1-2 bp deletions are particularly common repair outcomes. \hat{ }

Combined score=(Off-target score+Precision score*100+Frameshift)/3. † Pipe symbol ‘|’ indicates CRISPR cut site. Curly braces ‘{ }’ indicate insertion. Hyphen ‘-’ indicates deletion. $ Potential off-target sites. Scoring according to Hsu et al. (2013). The on-target site has a score of 100.

FIGS. 84A, 84B, 84C, and 84D collectively illustrate results of functional assays in edited or control canine chondrocytes measuring (A, B) cIL1A and (C,D) cIL1B release at 6 hours and 24 hours post-exposure to PBS or LPS.

FIGS. 85A, 85B, 85C, and 85D collectively illustrate results of functional assays in edited and control chondrocytes measuring (A, B) hIL1A and (C,D) cIL1B release from 6 hours and 24 hours after exposure to PBS or LPS.

FIG. 86 illustrates the results of a tissue-specific splicing and expression analysis of the hIL1A gene.

FIG. 87 illustrates the results of a tissue-specific splicing and expression analysis of hIL1B gene.

FIG. 88 illustrates the results of an in silico analysis of crRNAs targeting either hIL1A or hIL1B. On-target score (see Doench et al.) is optimized for 20-bp gRNA with NGG protospacer adjacent motif (PAM). Score spans from 0 to 1. Precision score is based on experiments in U2OS cells. A high precision score (>0.4) implies that DNA repair outcomes are uniform and enriched for just a handful of unique genotypes. Frameshift percentage is based on experiments in U2OS cells. A high (>80%) frameshift frequency will tend to knock a protein-coding gene out of frame. The typical genomic frameshift frequency is above 66% because 1-bp insertions and 1-2 bp deletions are particularly common repair outcomes. Off-target score from CRISPR assess the number of matches in the genome with a given number of mismatches. Mismatches in Seed sequence have a more deleterious effect.

FIG. 89 illustrates results of splicing and functional analyses on cIL1A and cIL1B genes. The reference canine genome assembly (CanFam3.1) was used for these analyses.

FIG. 90 illustrates the results of an in silico analysis of crRNAs targeting either cIL1A or cIL1B genes.

FIGS. 91A, 91B, and 91C collectively illustrate knockdown efficacy of selected sgRNAs in (A) human chondrocytes, (B) canine chondrocytes and (C) canine synoviocytes.

FIG. 92 illustrates the results of an in silico analysis of the off-target effects for multiple sgRNAs in canine cells.

FIGS. 93A and 93B collectively illustrate (A) the efficacy of enhanced-specificity Cas9 (espCas9) to abrogate the off-target editing of the indicated sgRNA in canine cells as compared to (B) the effects with canonical spCas9.

FIGS. 94A and 94B collectively illustrate summaries of editing activity for crRNAs targeting IL1A and IL1B in (A) humans and (B) canine chondrocytes.

FIG. 95 illustrates results of co-administrating multiple sgRNAs in canine cells either simultaneously or sequentially.

FIGS. 96A and 96B collectively illustrate sequence alignments of (A) IL1A and (B) IL1B genes for disparate mammalian species (human, horse, mouse and dog).

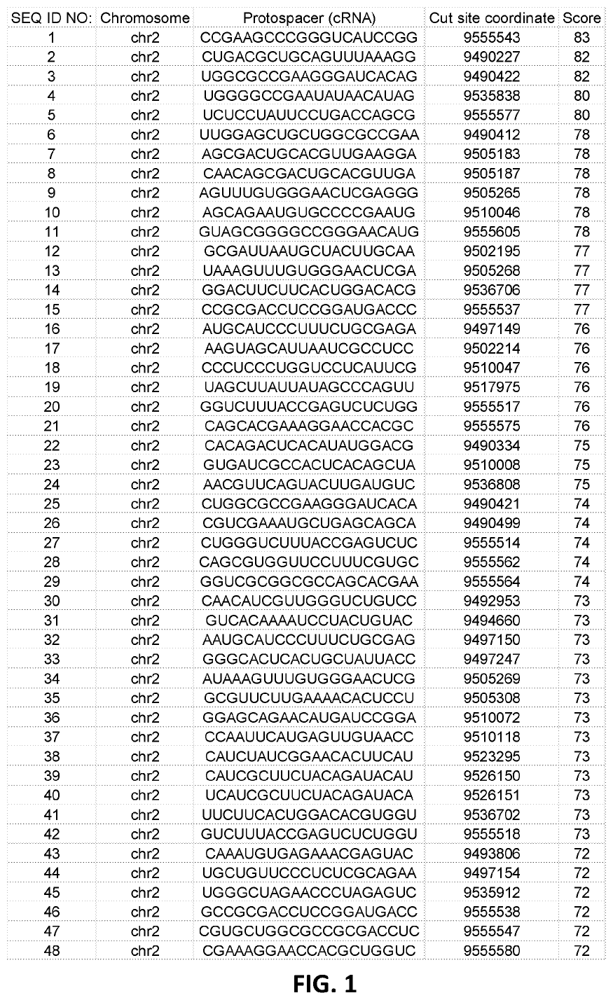

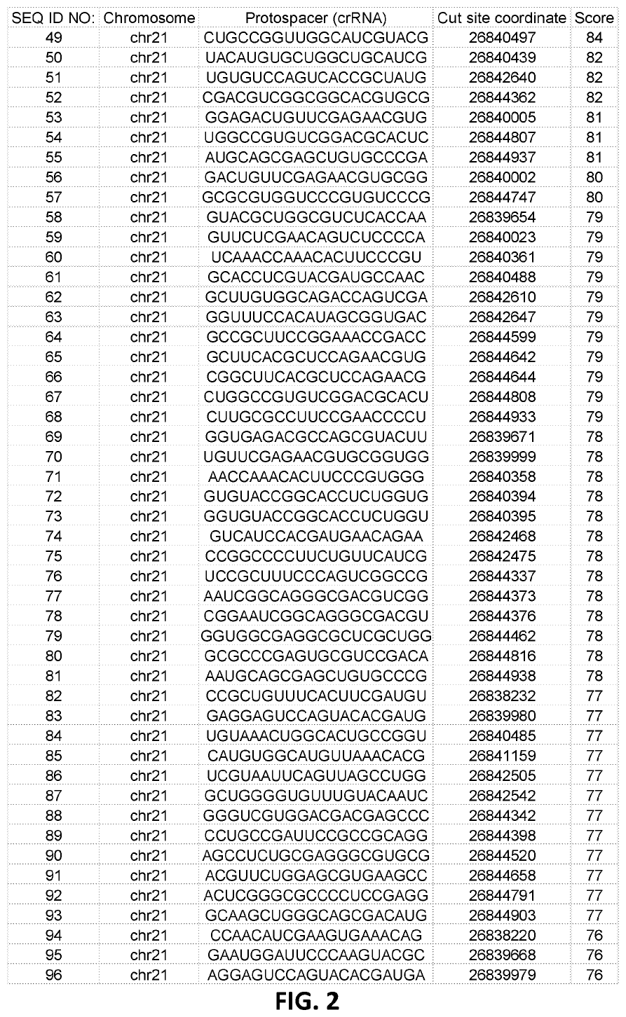

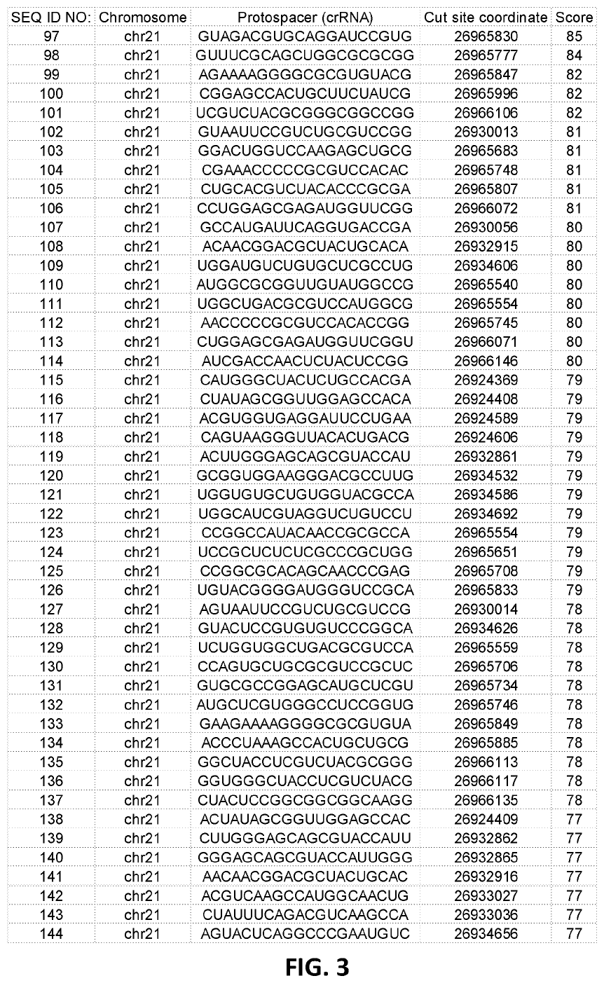

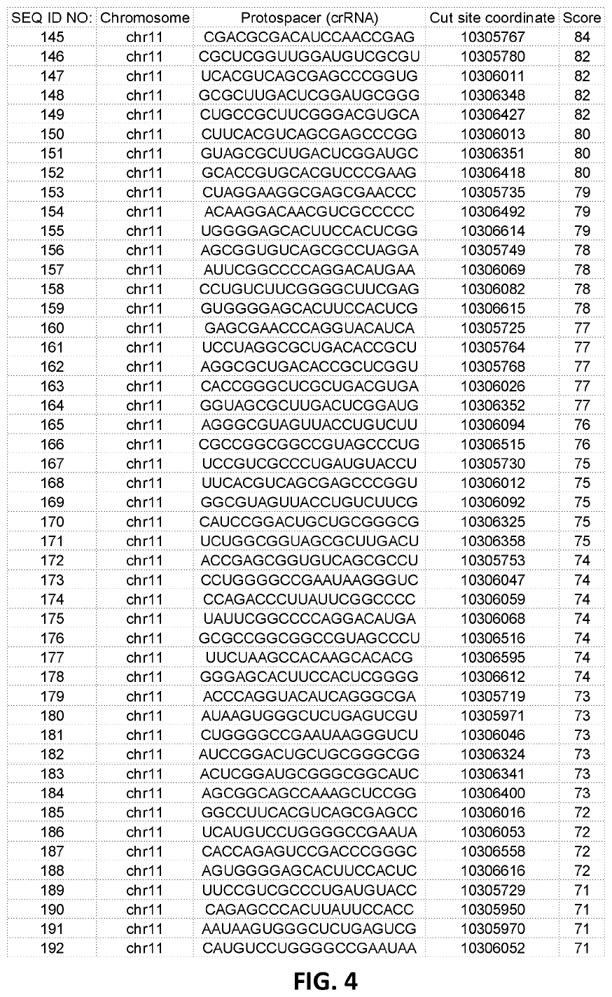

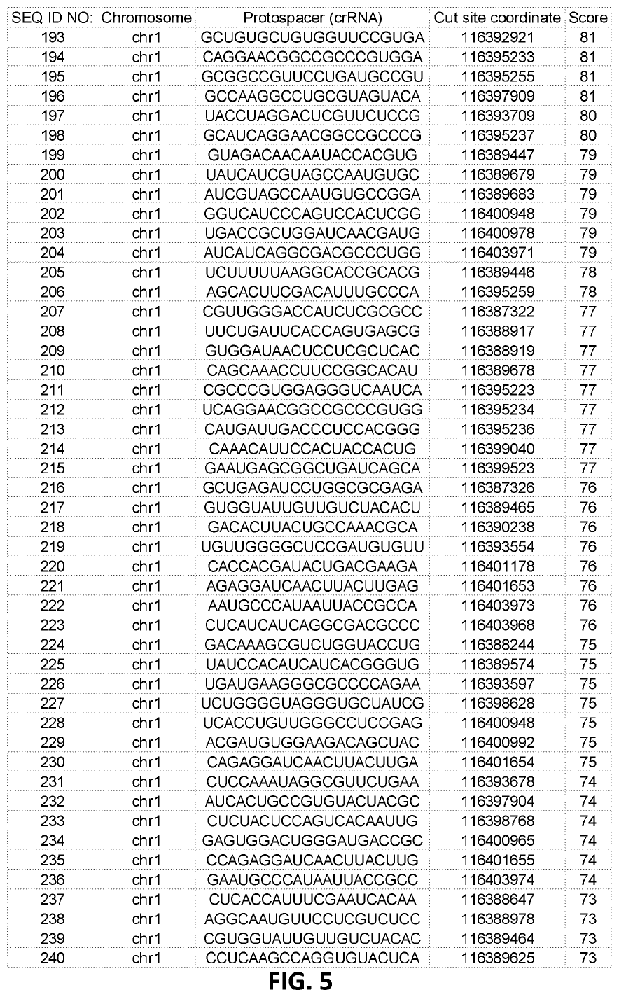

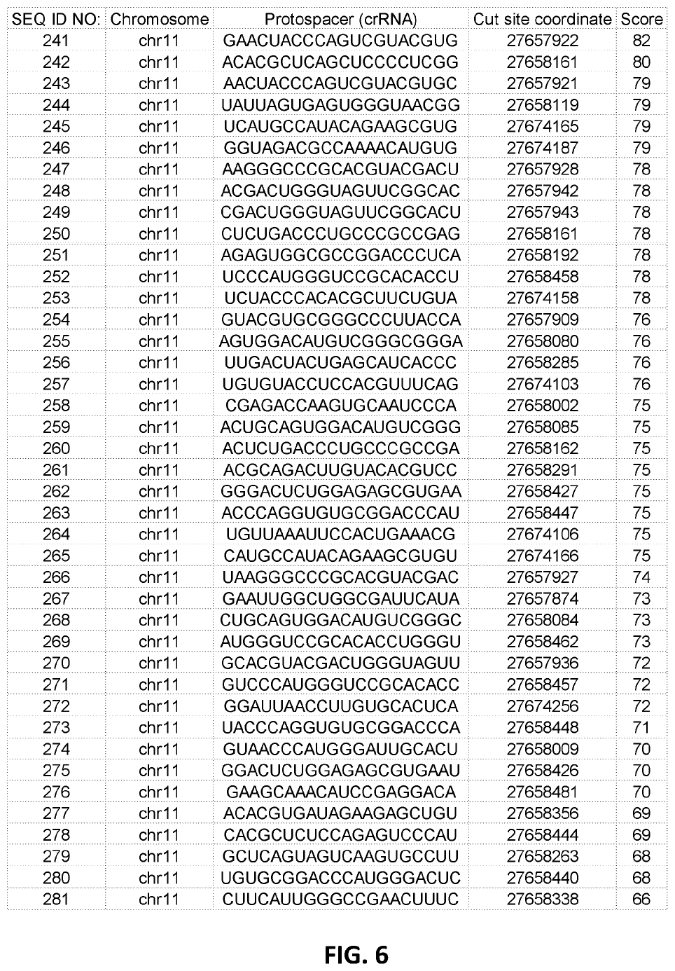

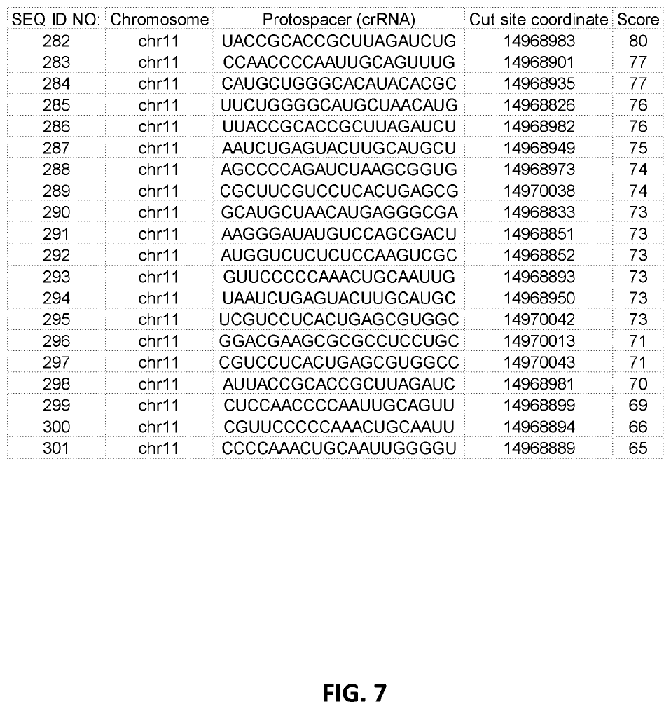

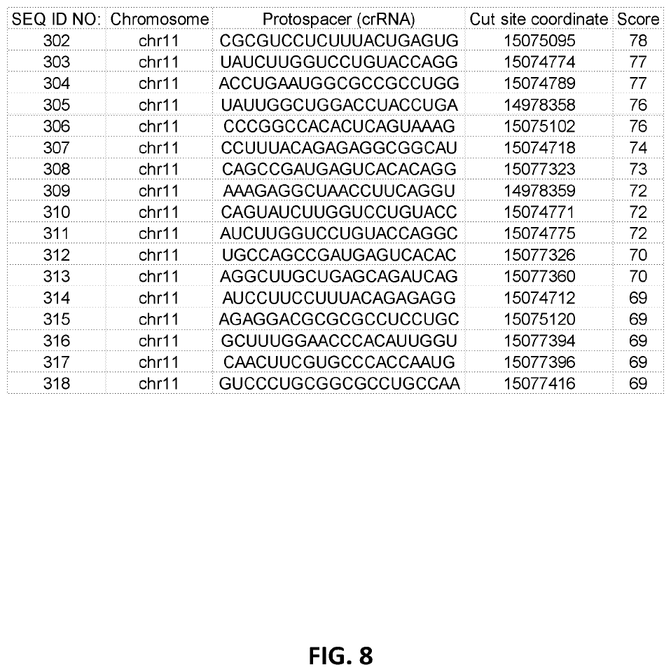

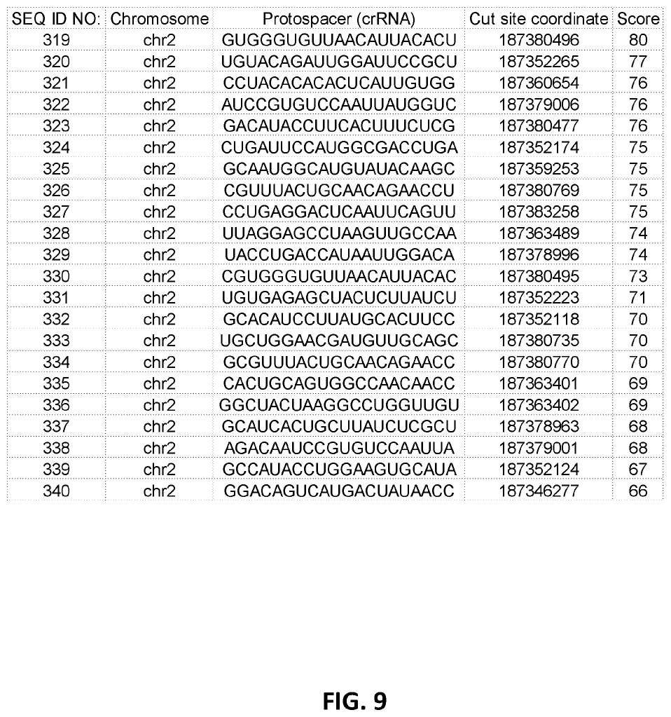

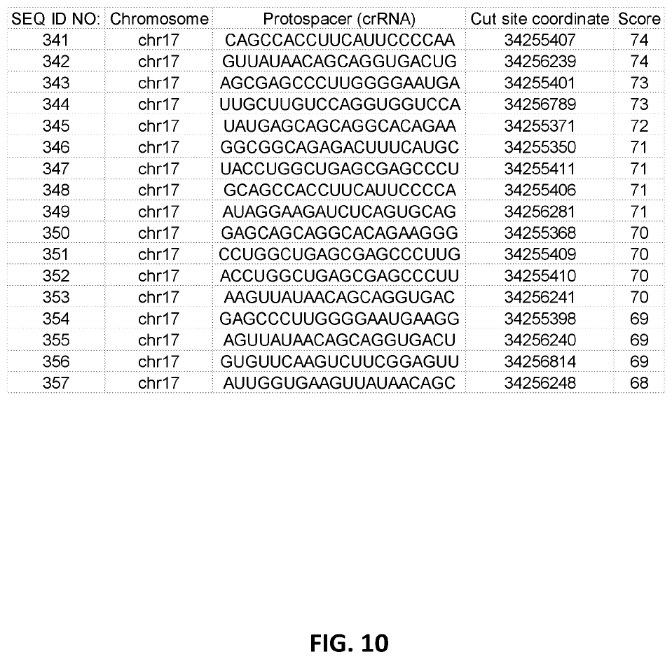

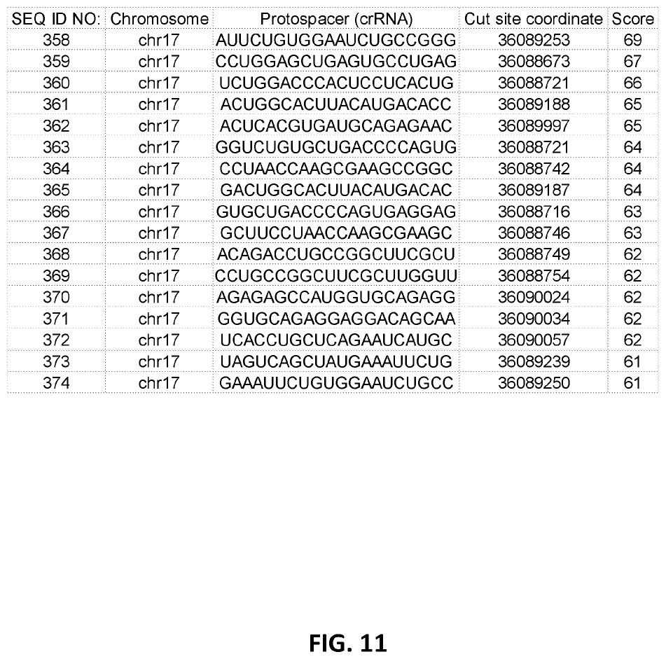

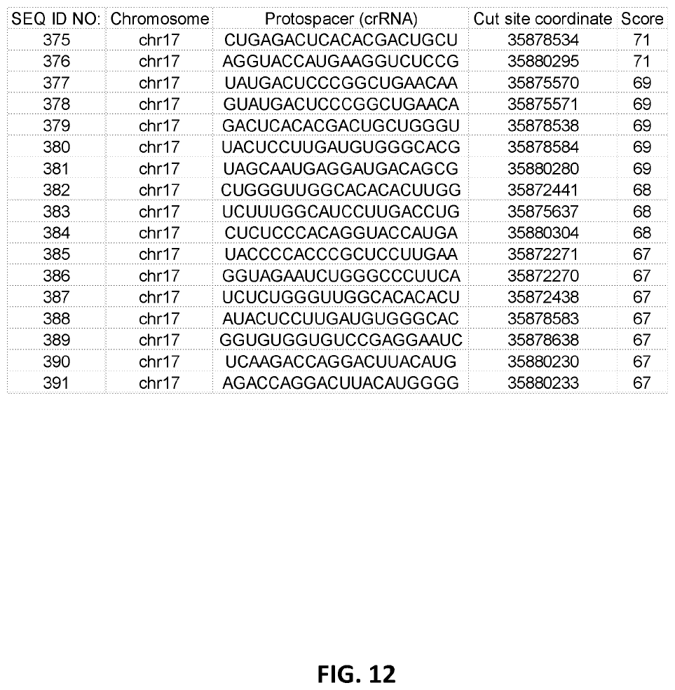

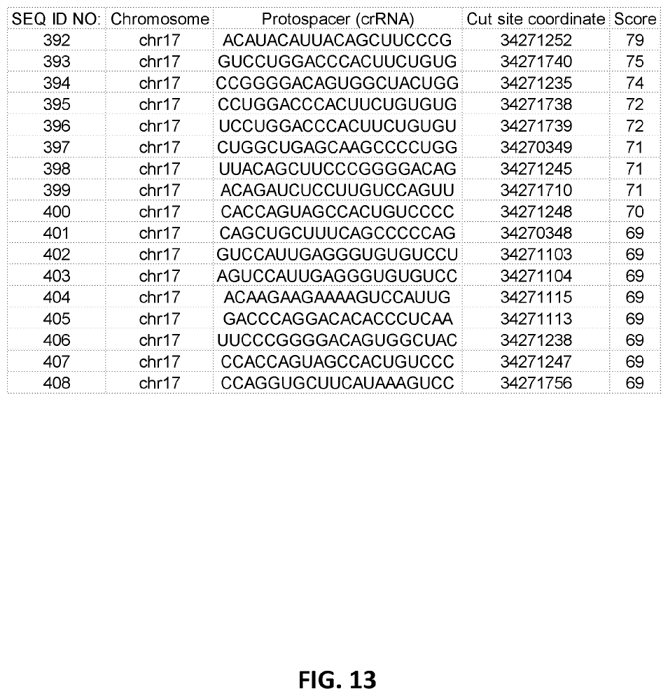

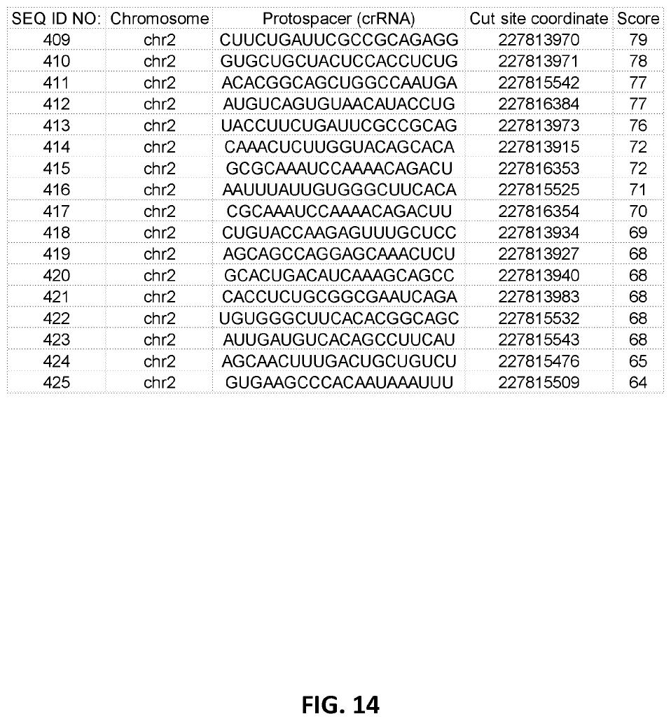

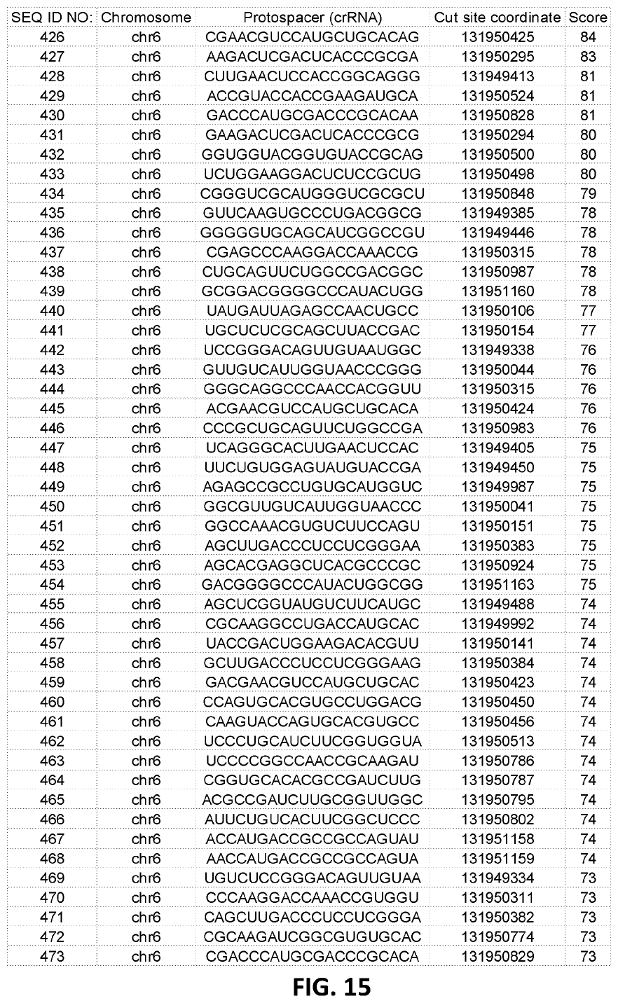

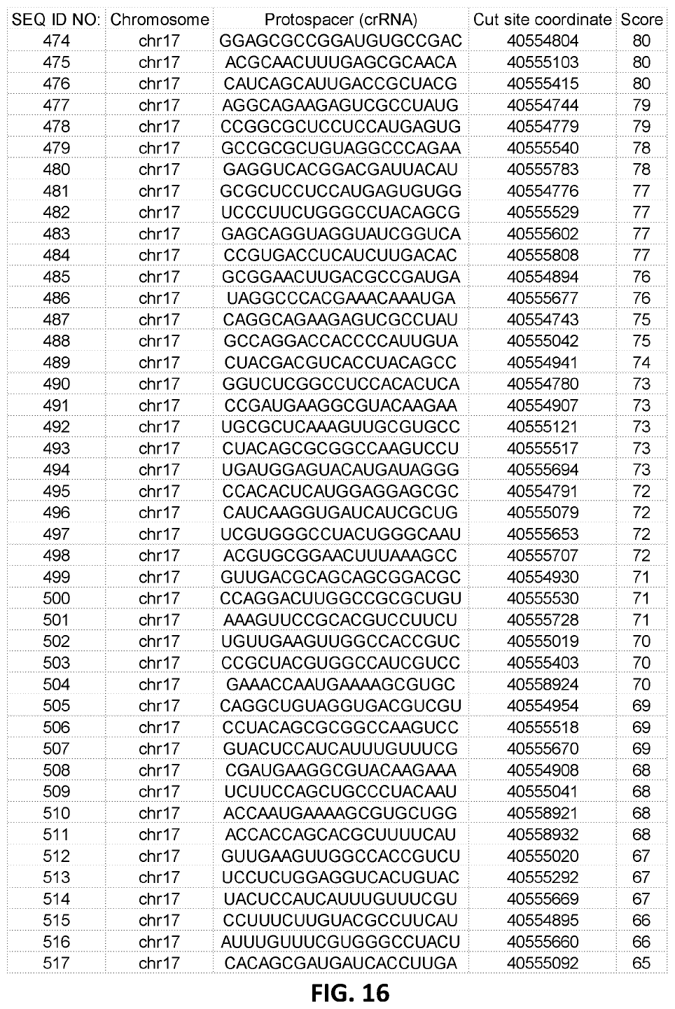

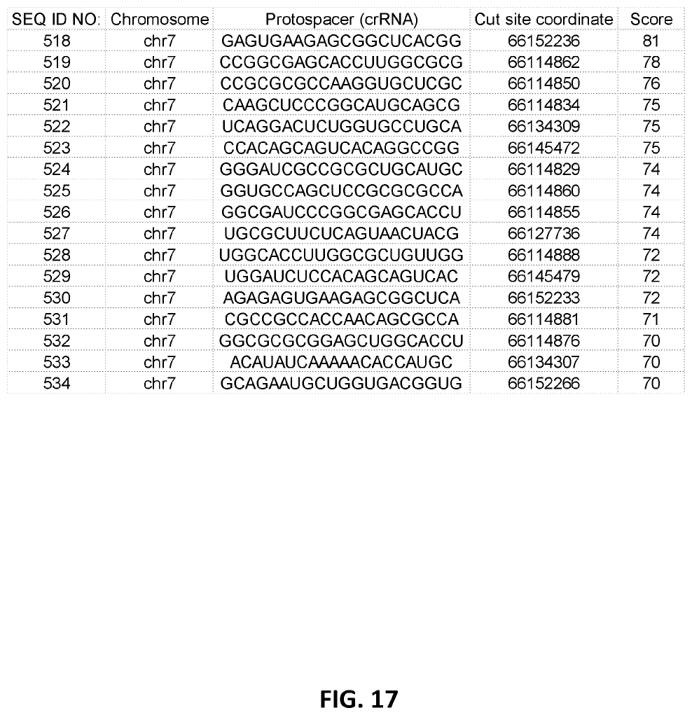

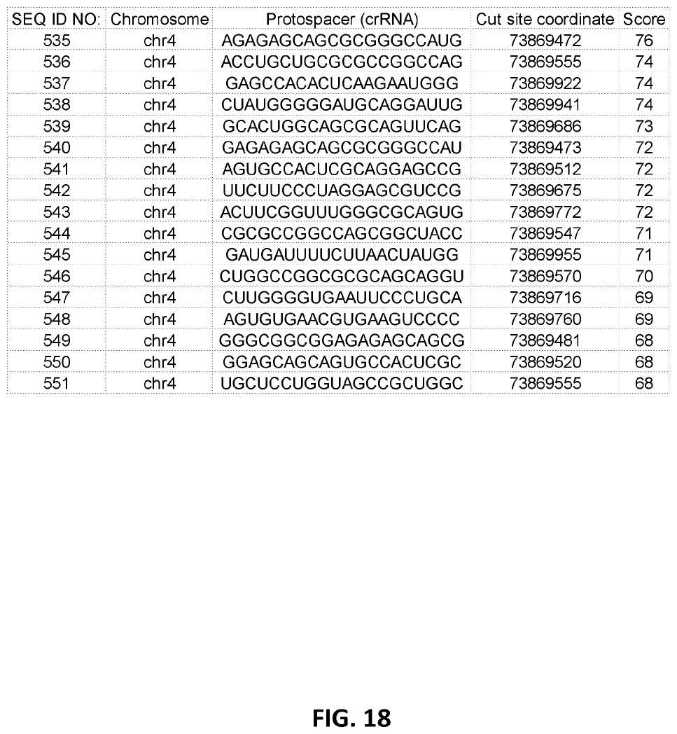

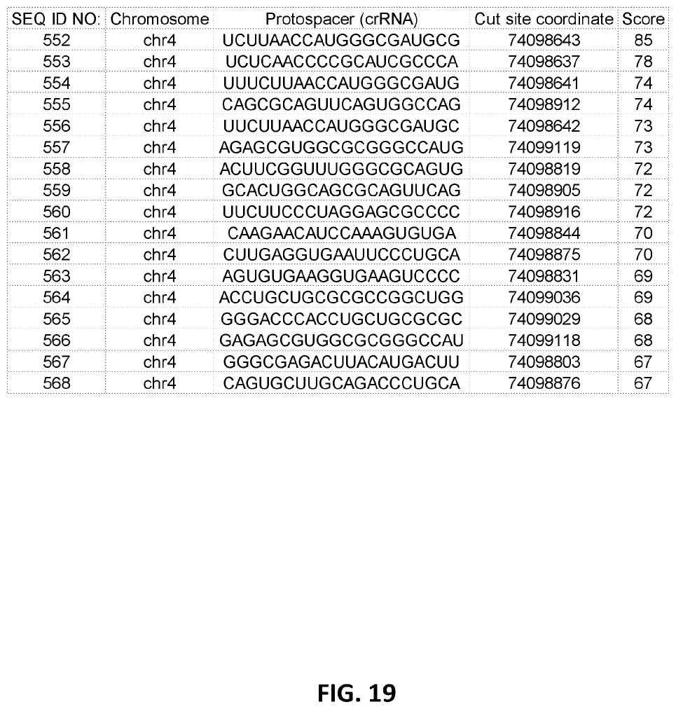

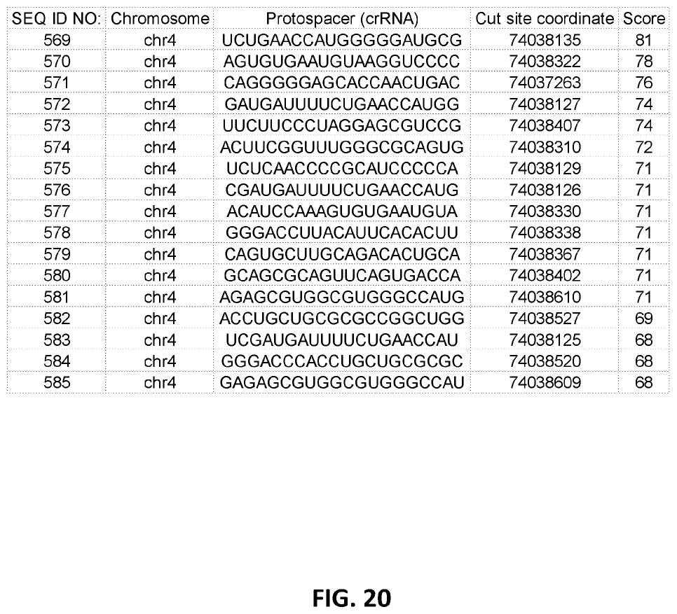

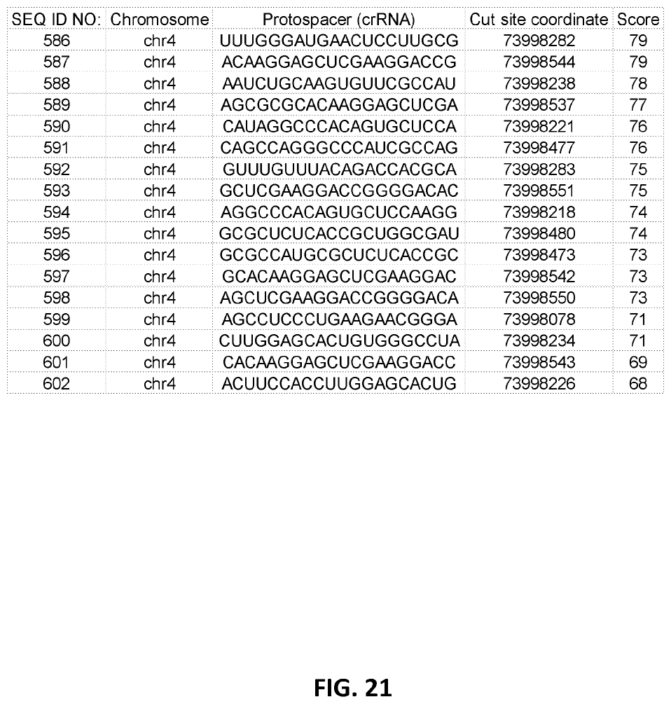

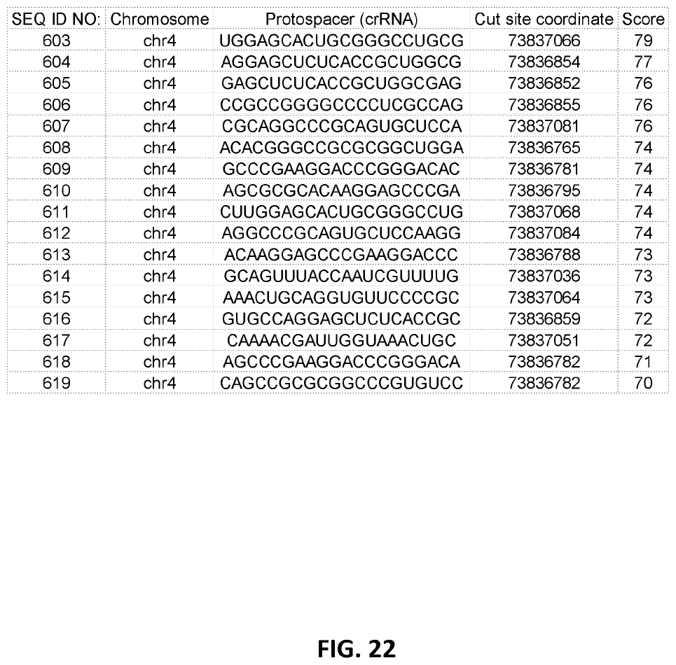

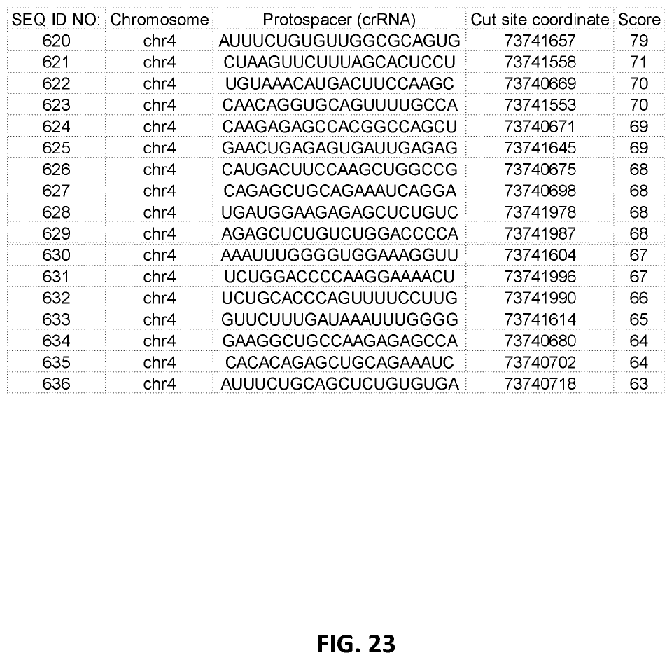

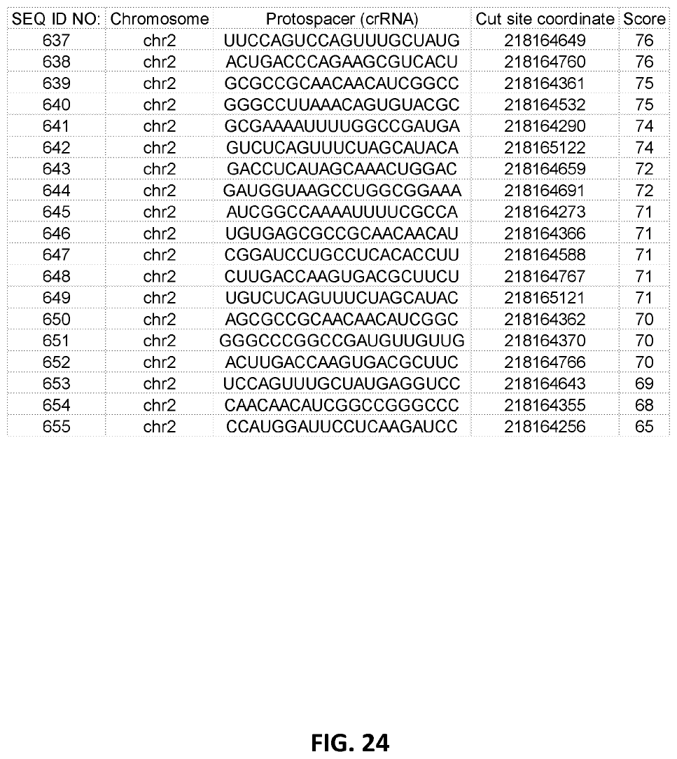

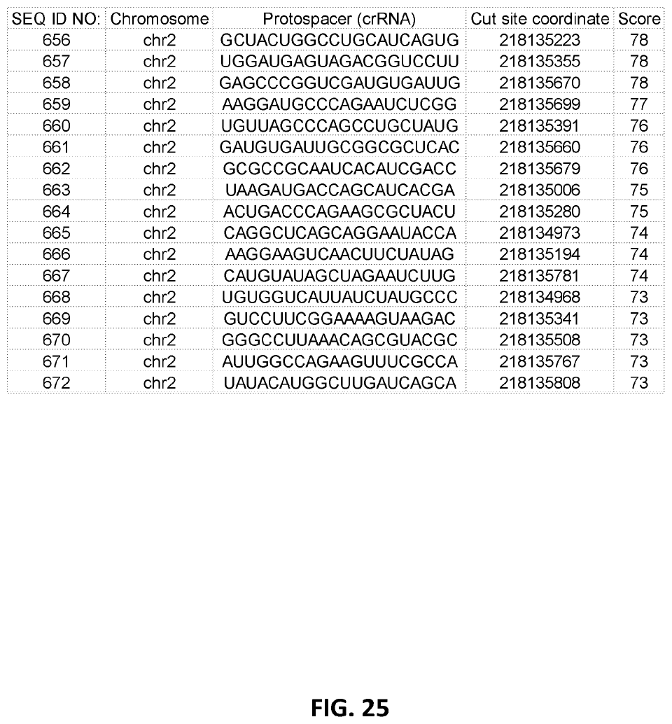

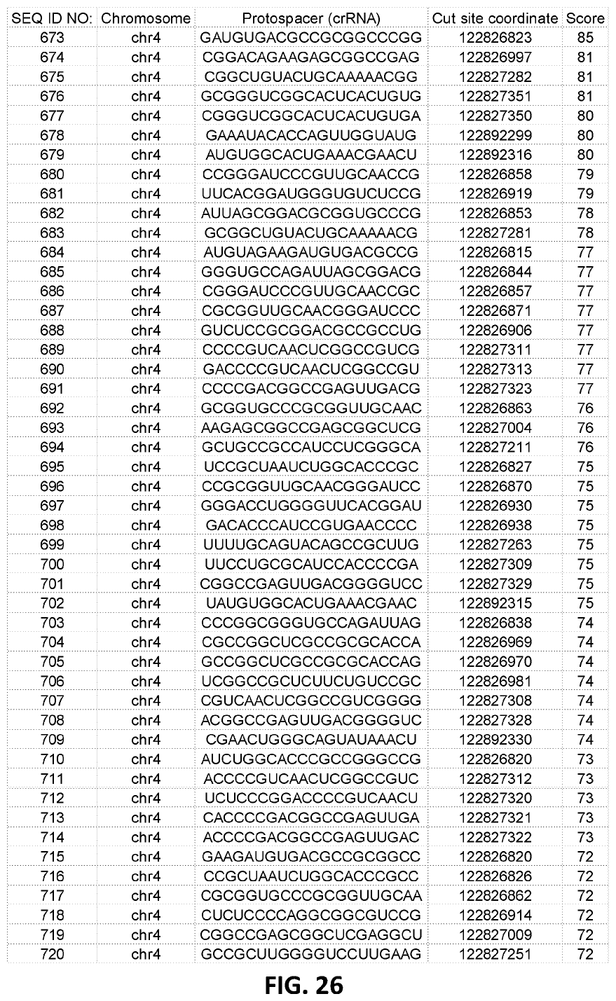

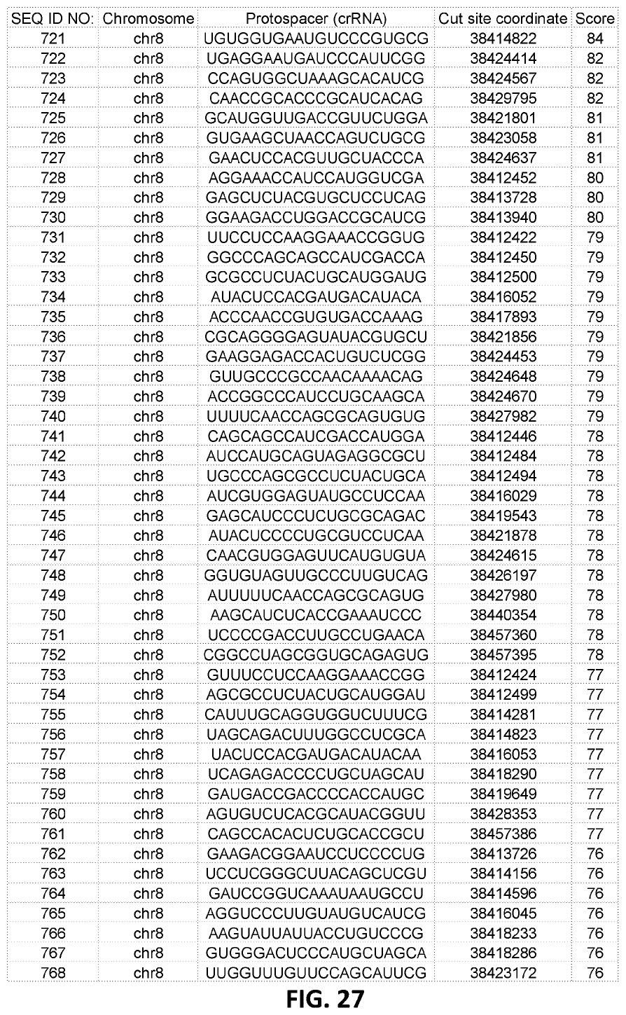

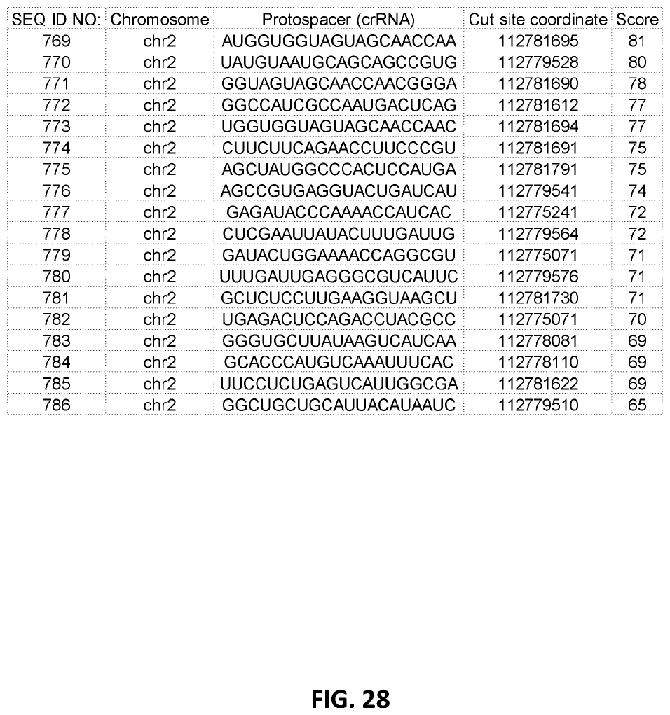

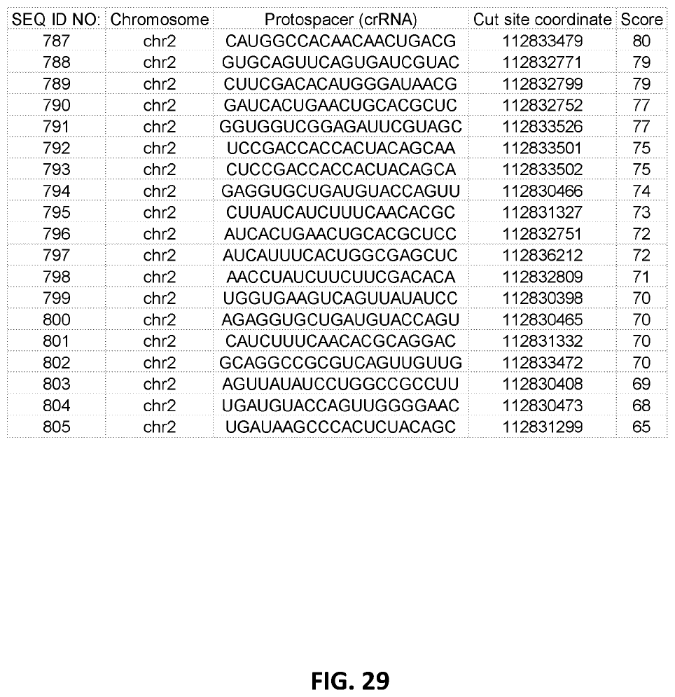

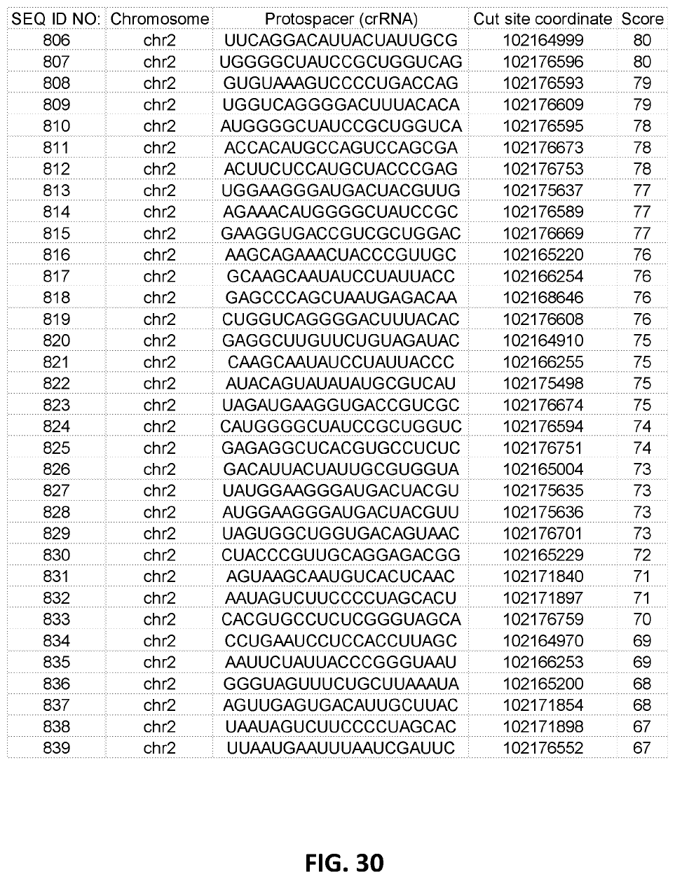

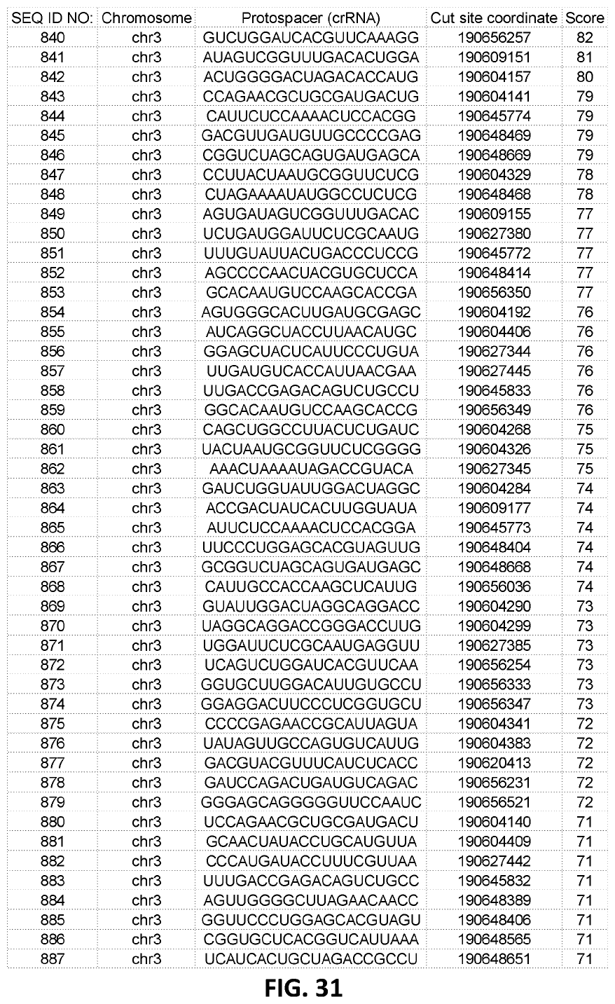

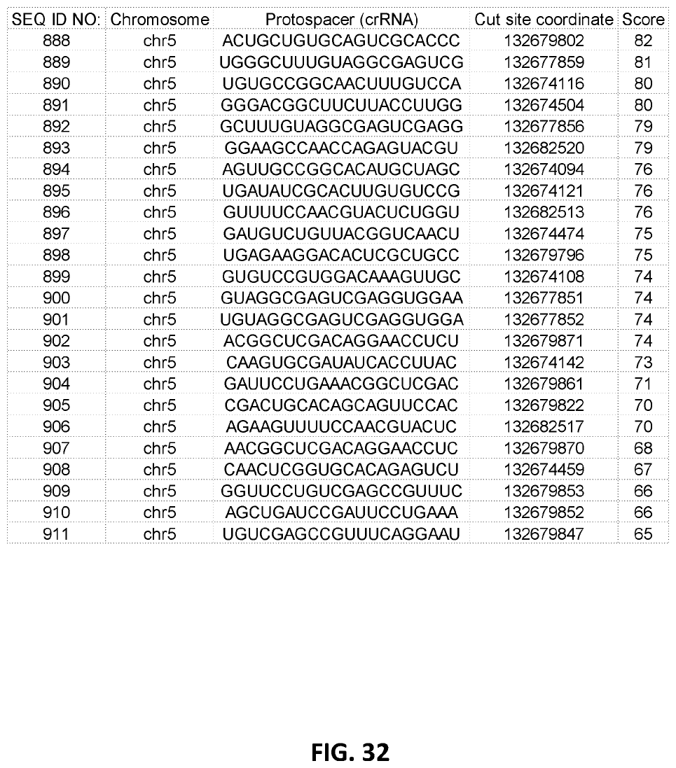

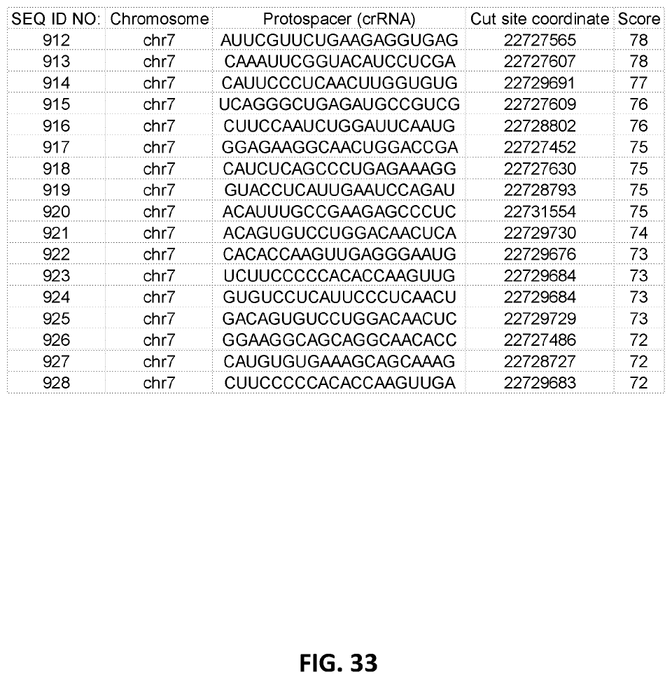

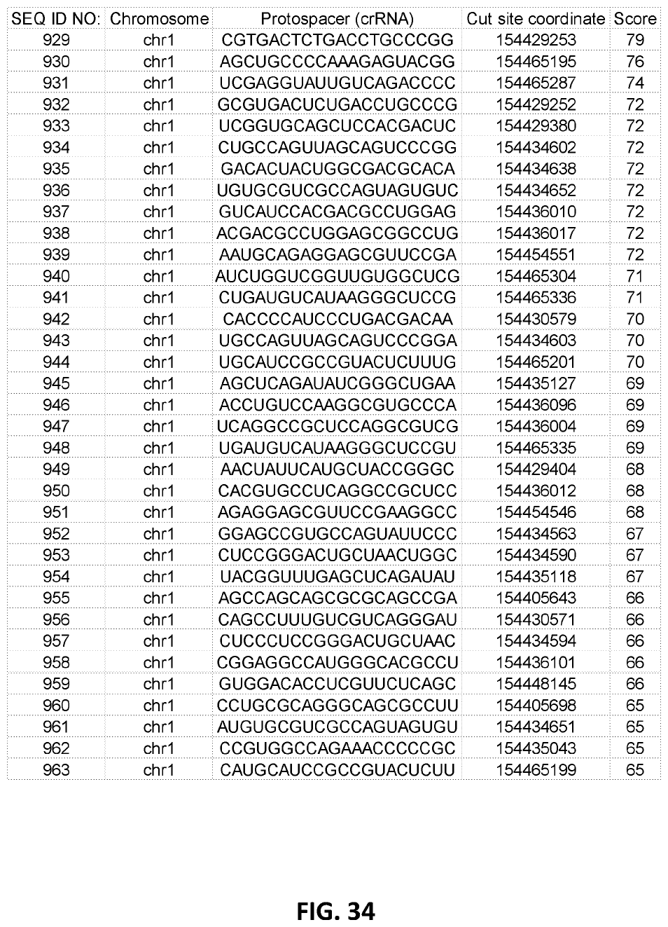

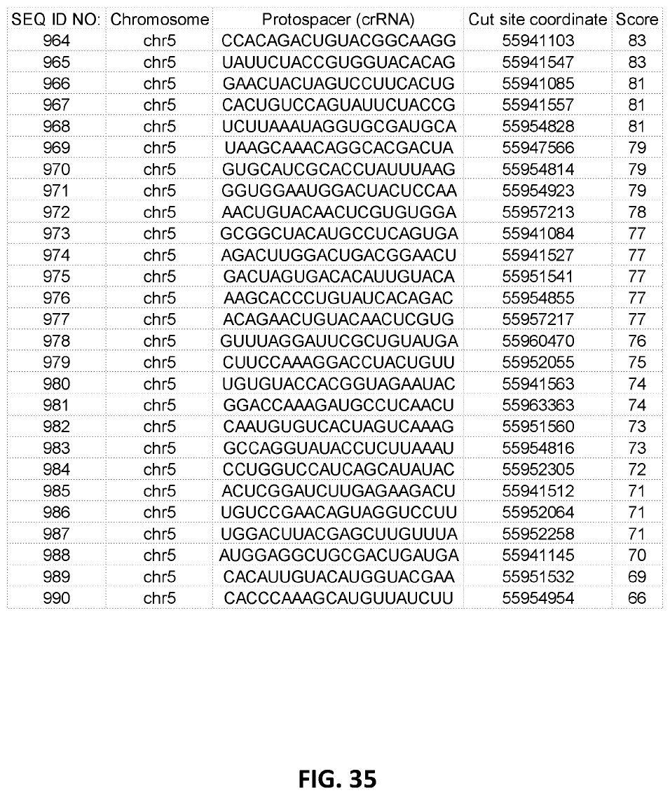

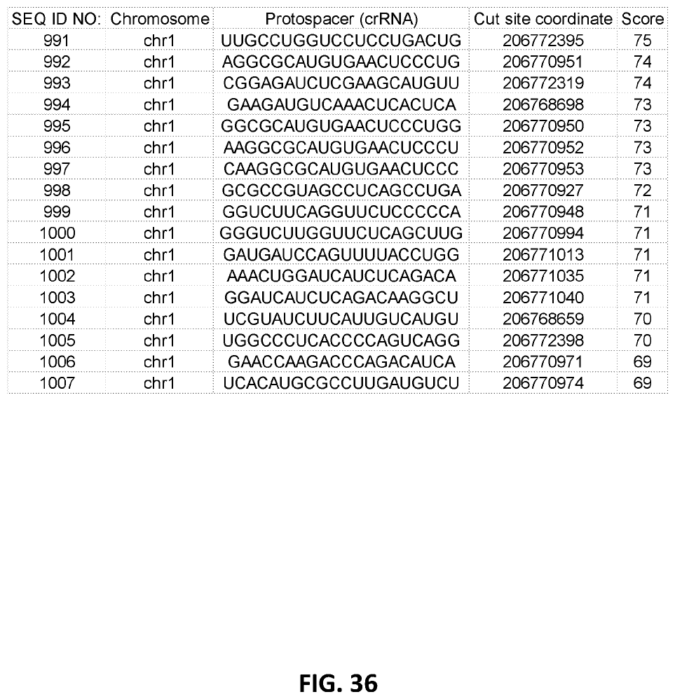

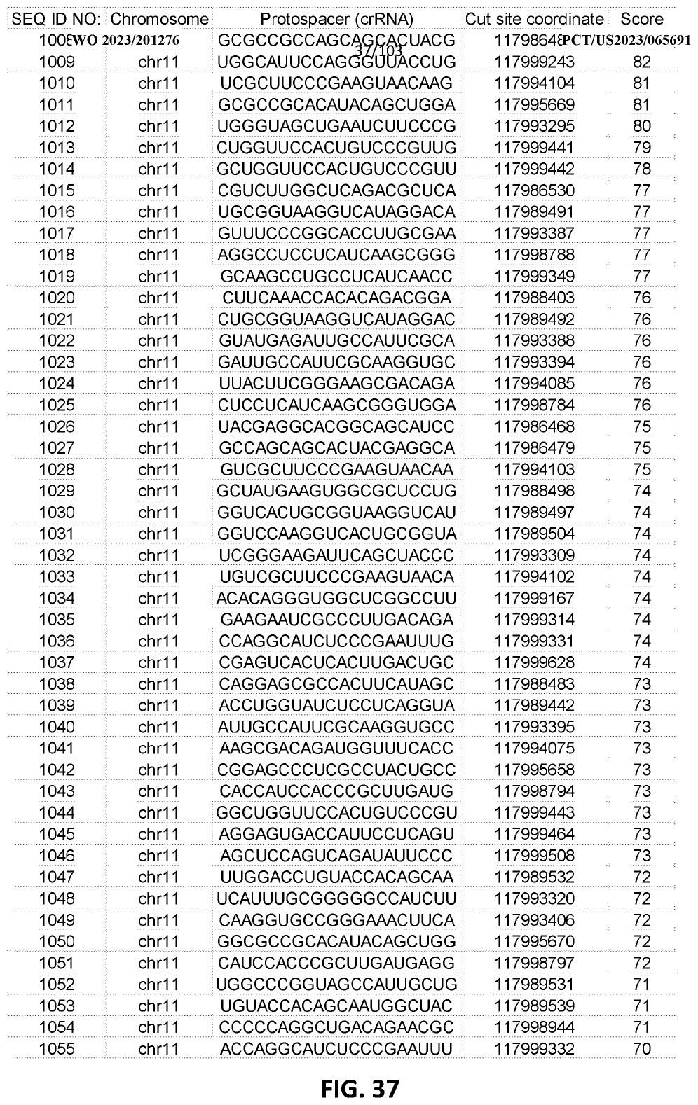

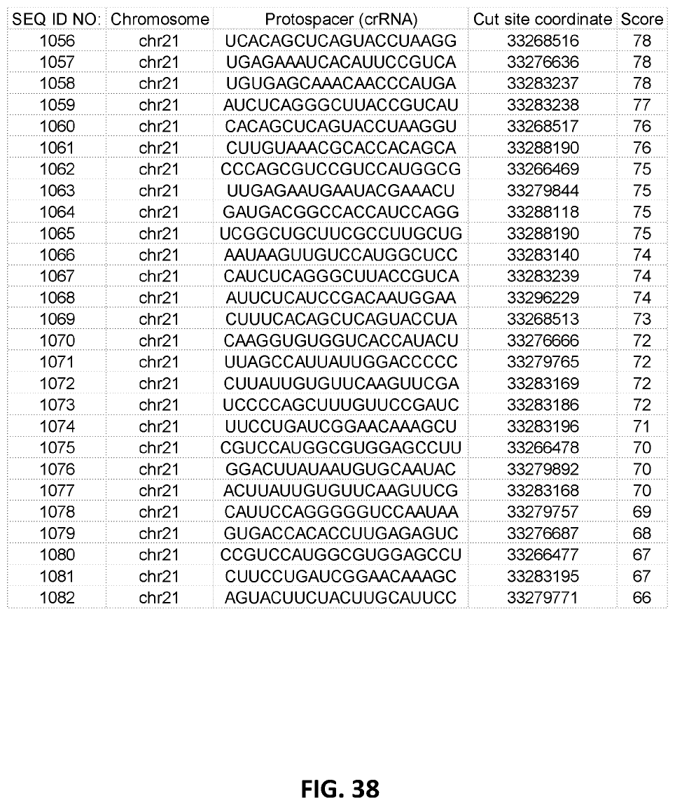

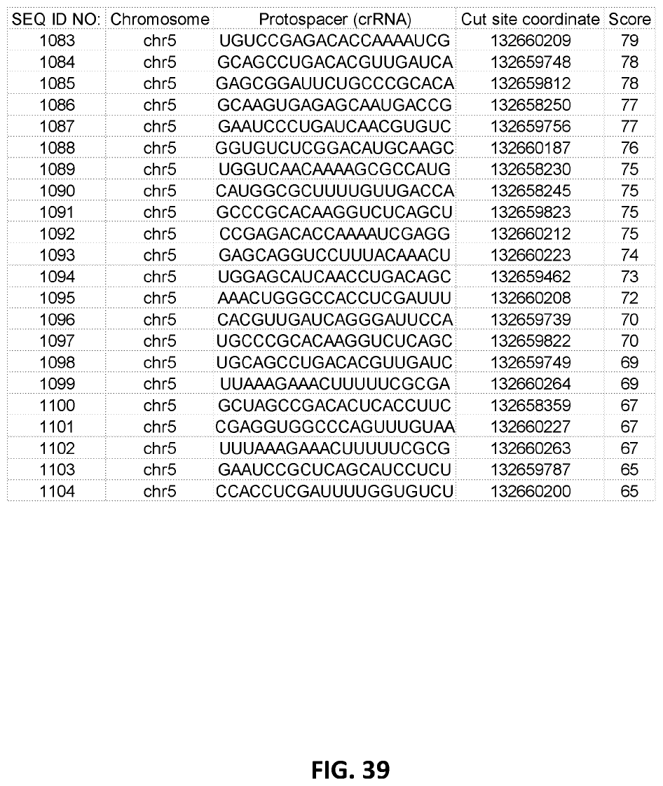

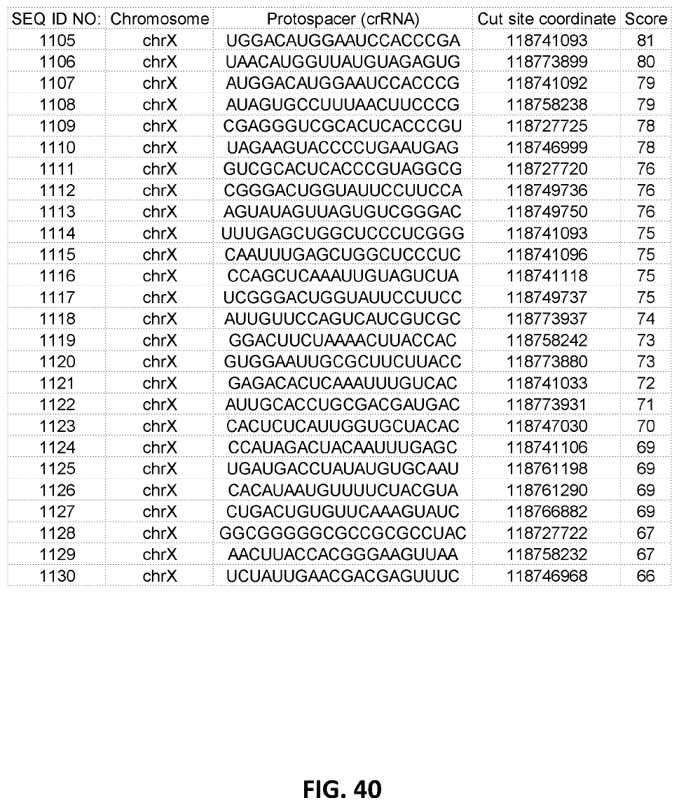

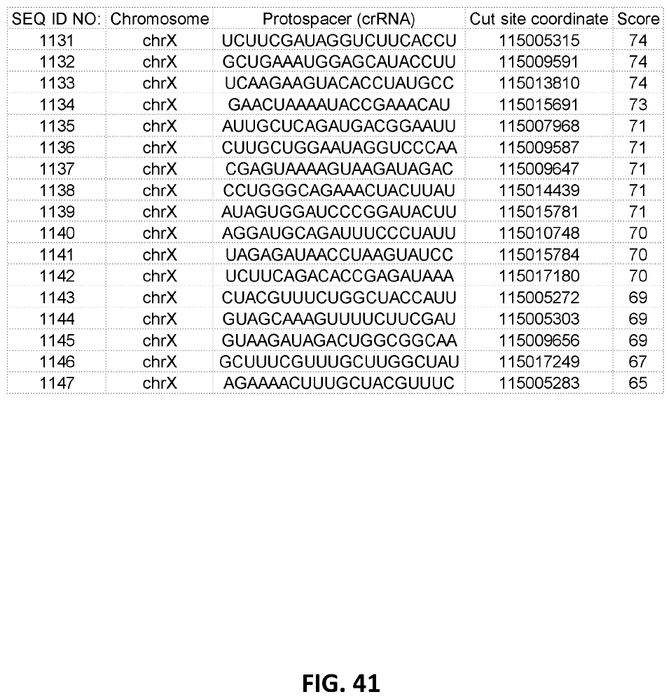

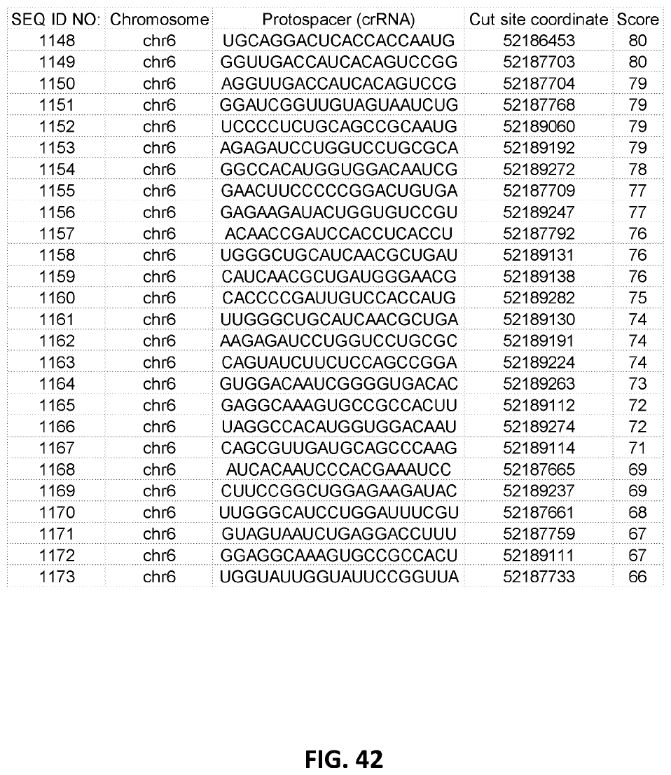

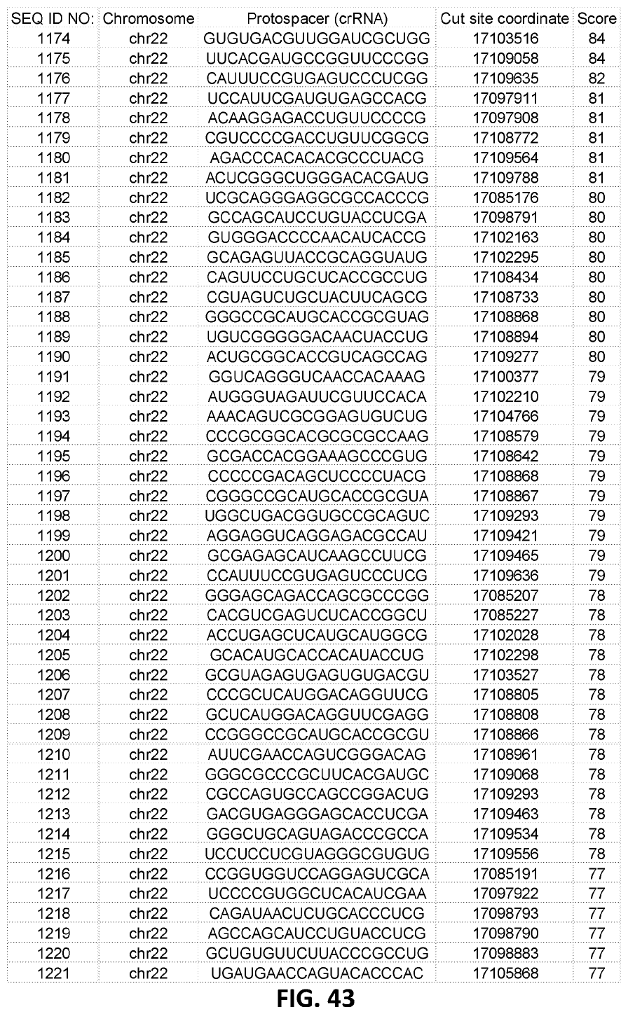

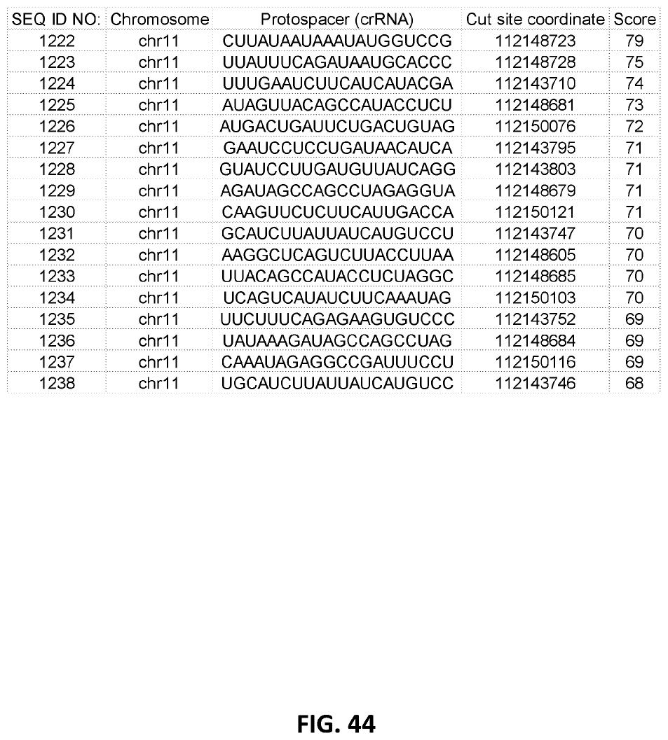

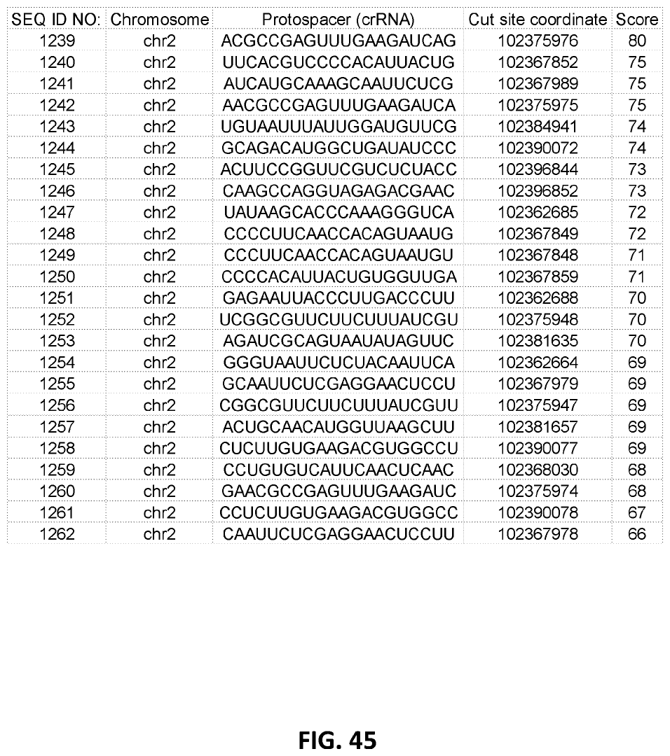

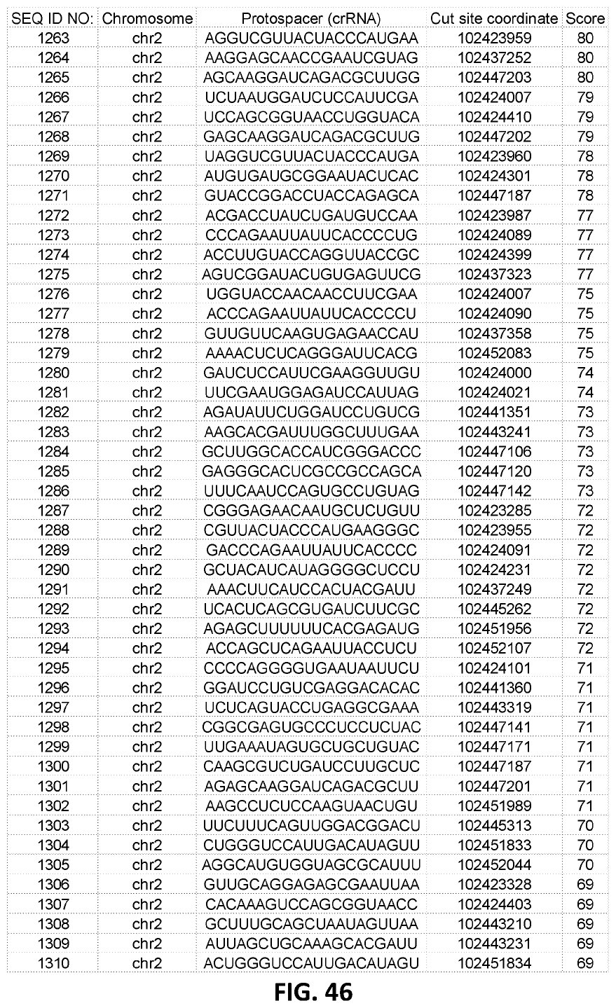

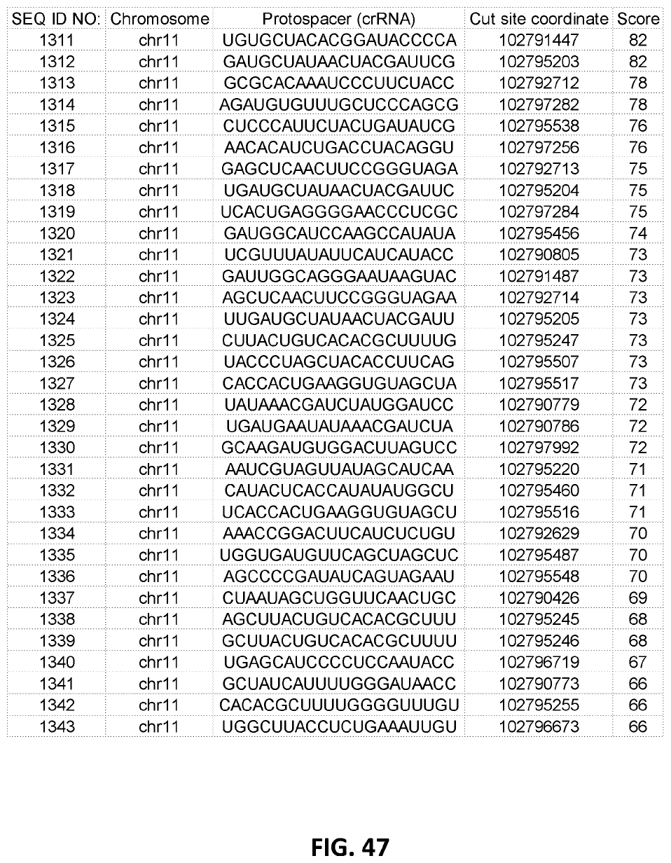

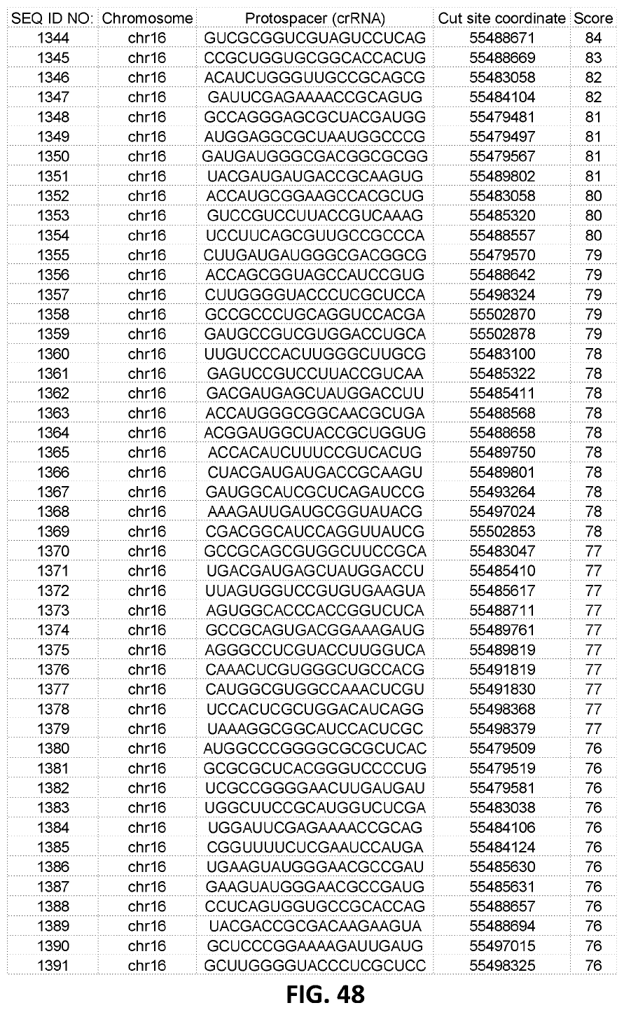

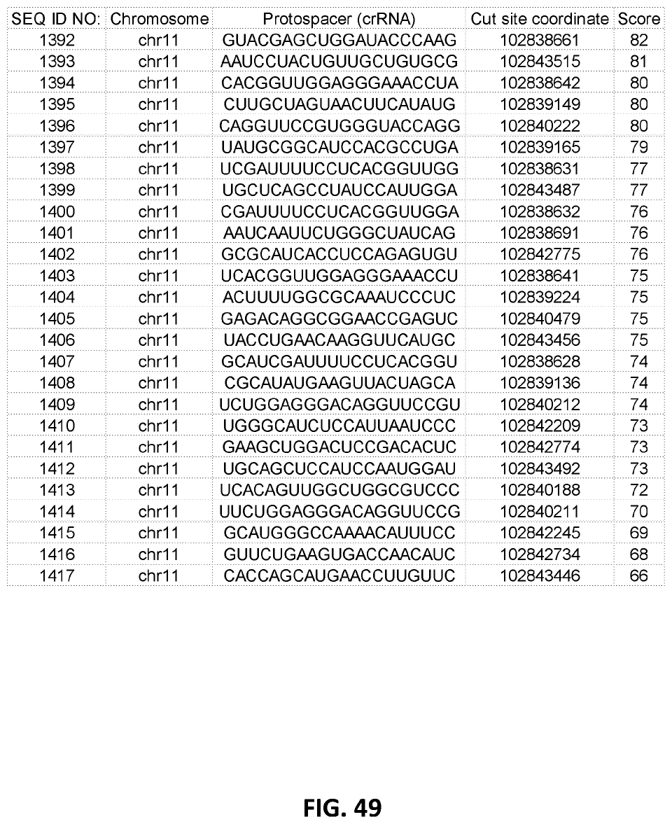

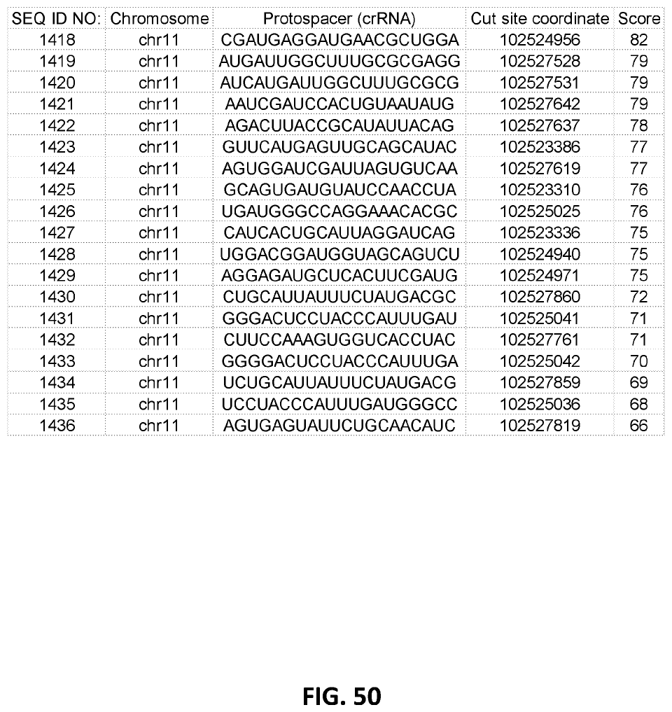

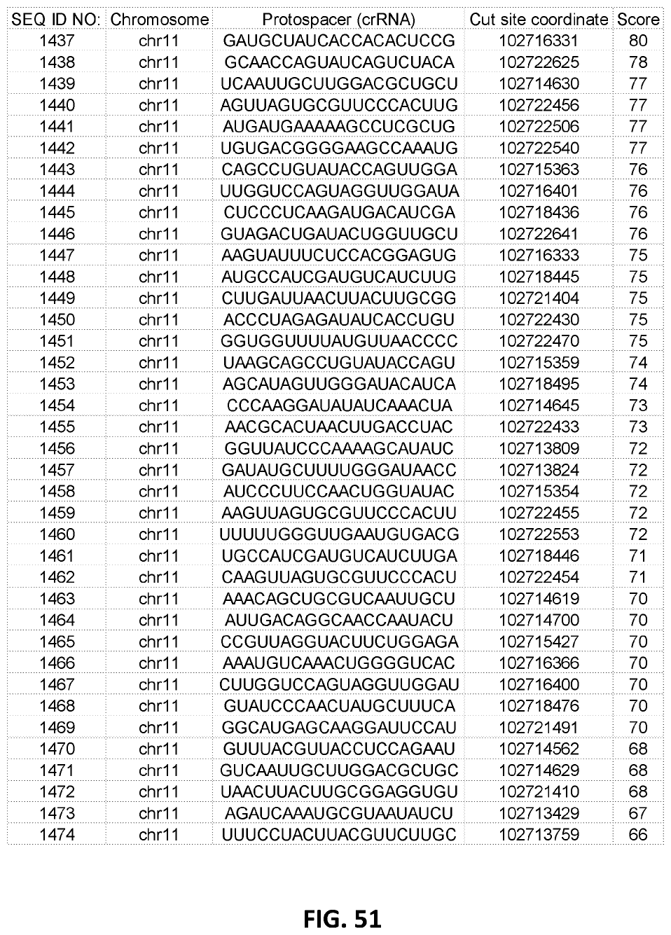

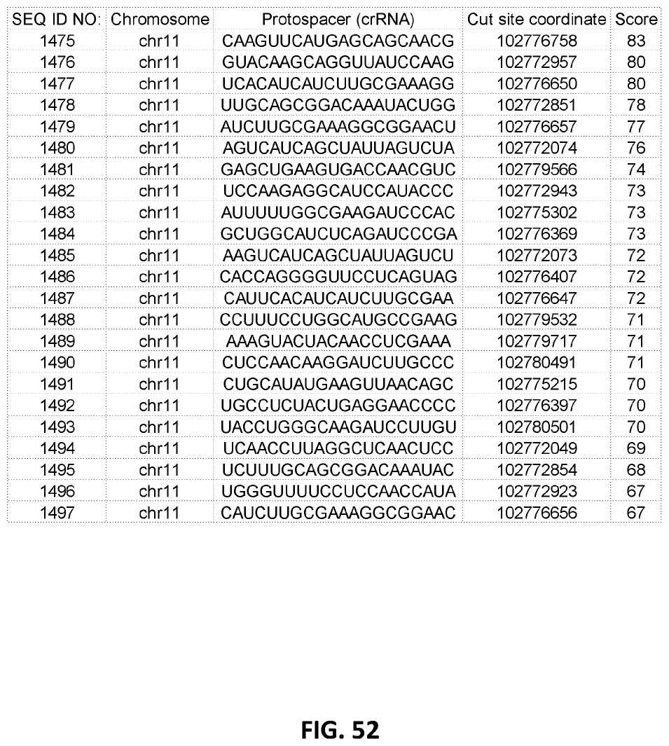

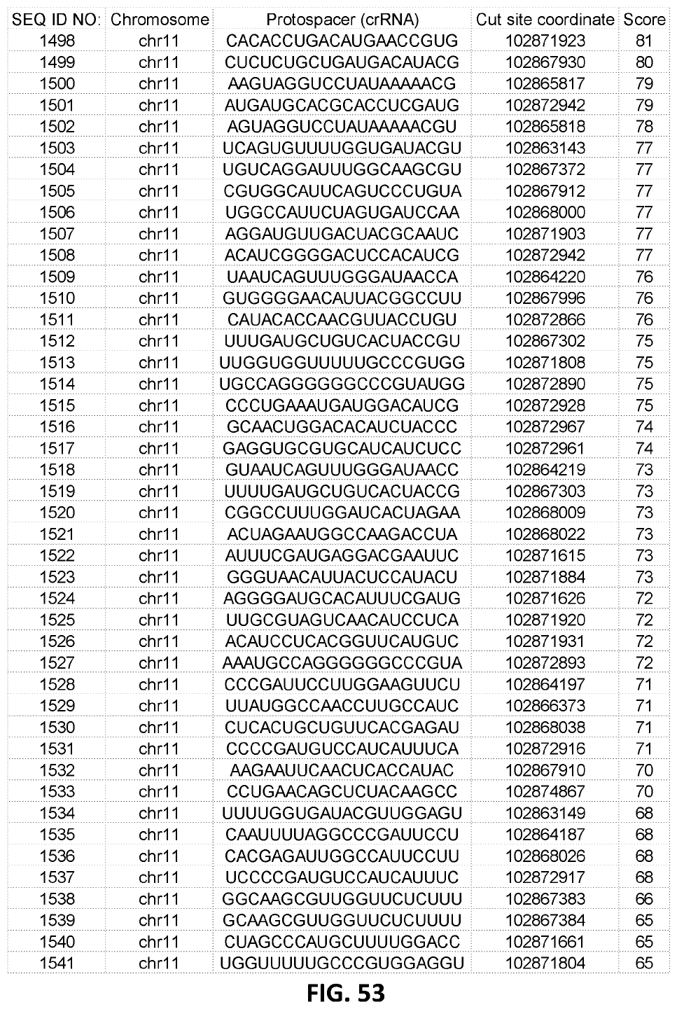

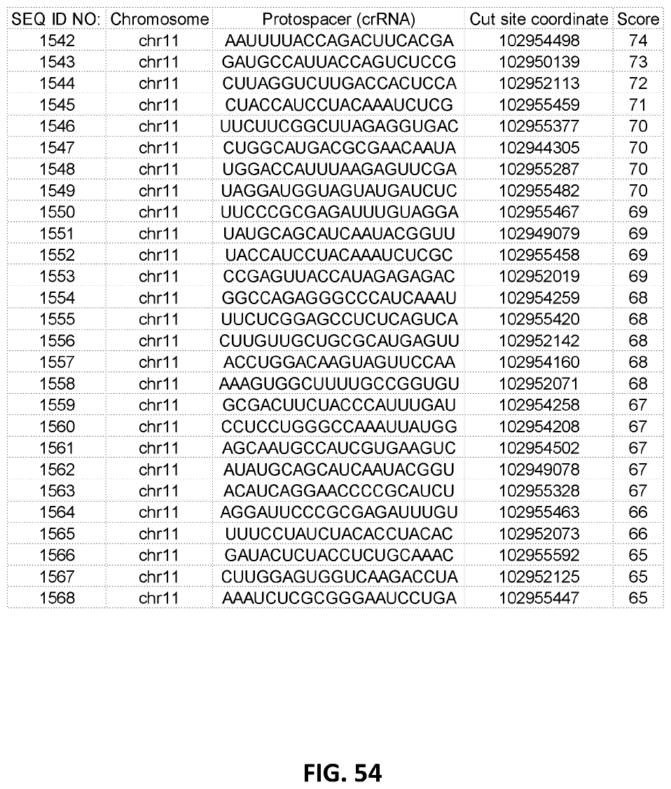

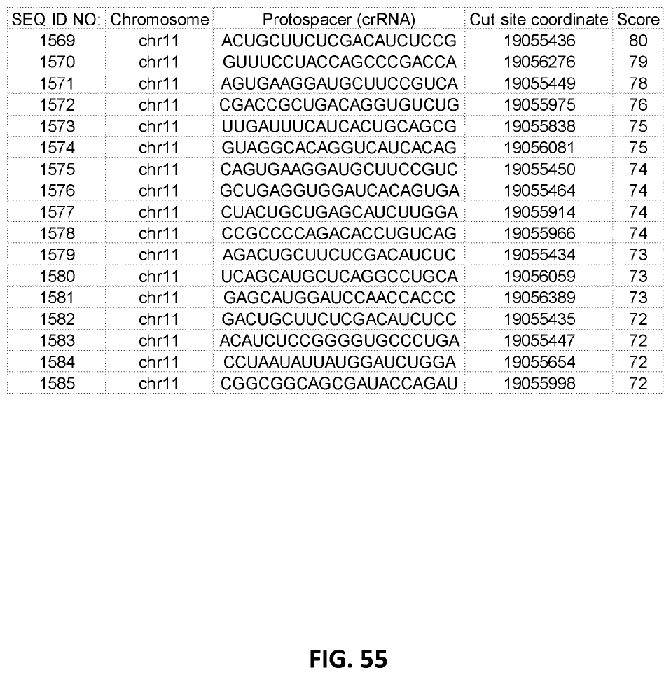

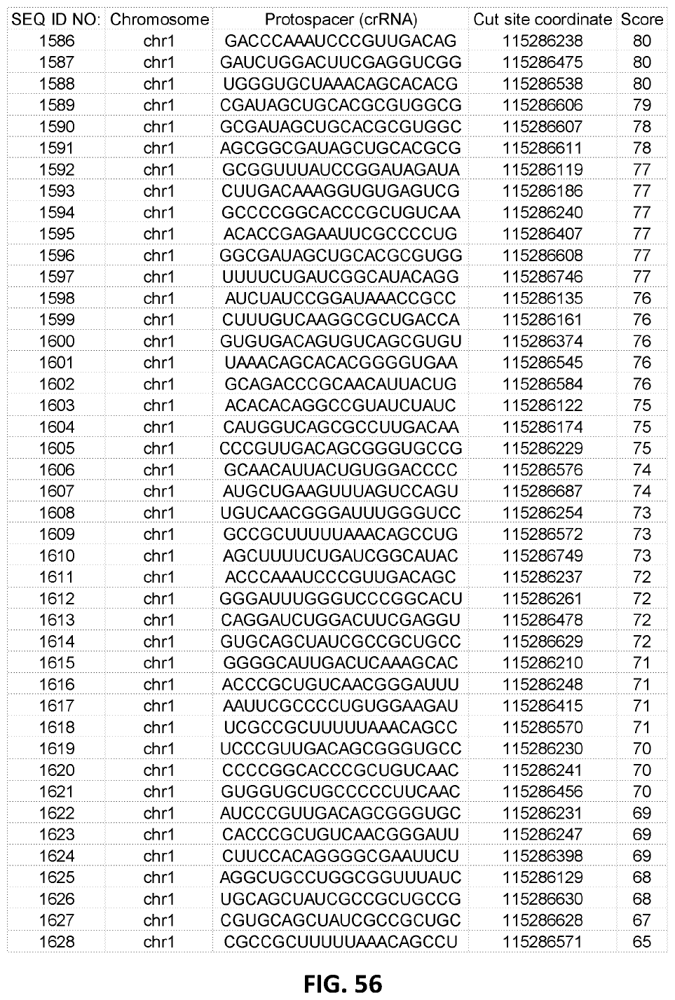

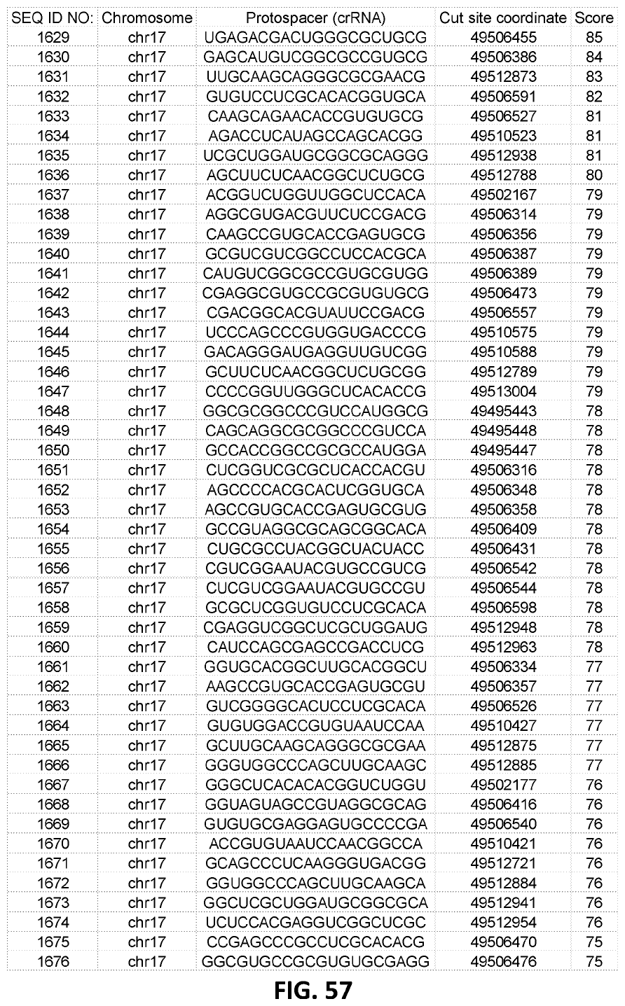

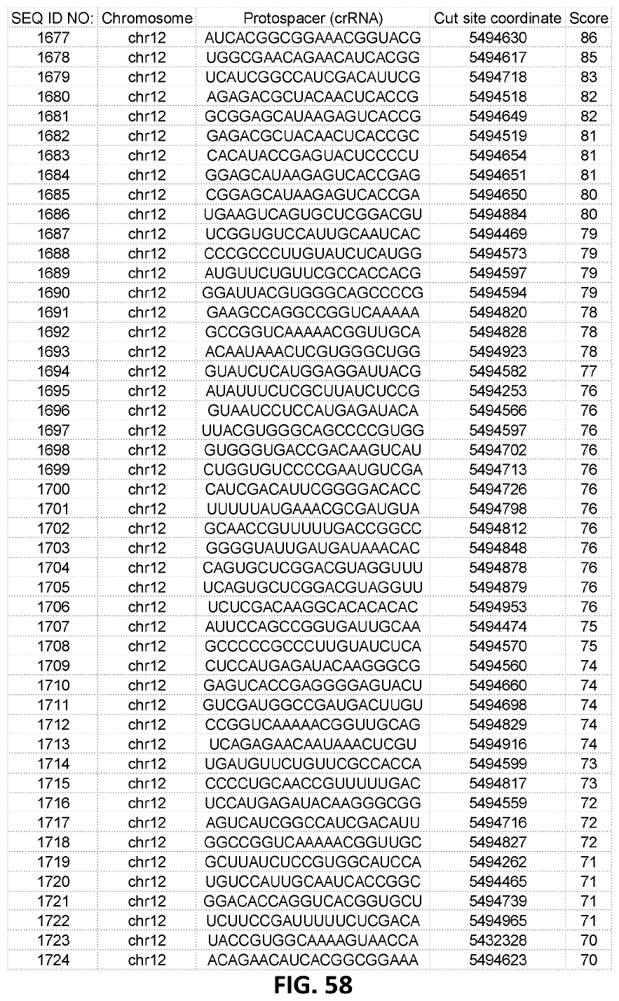

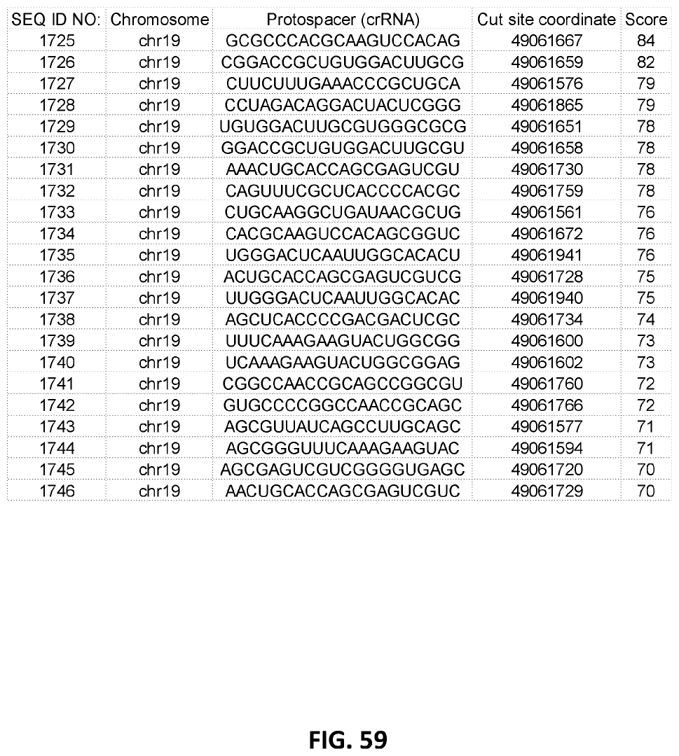

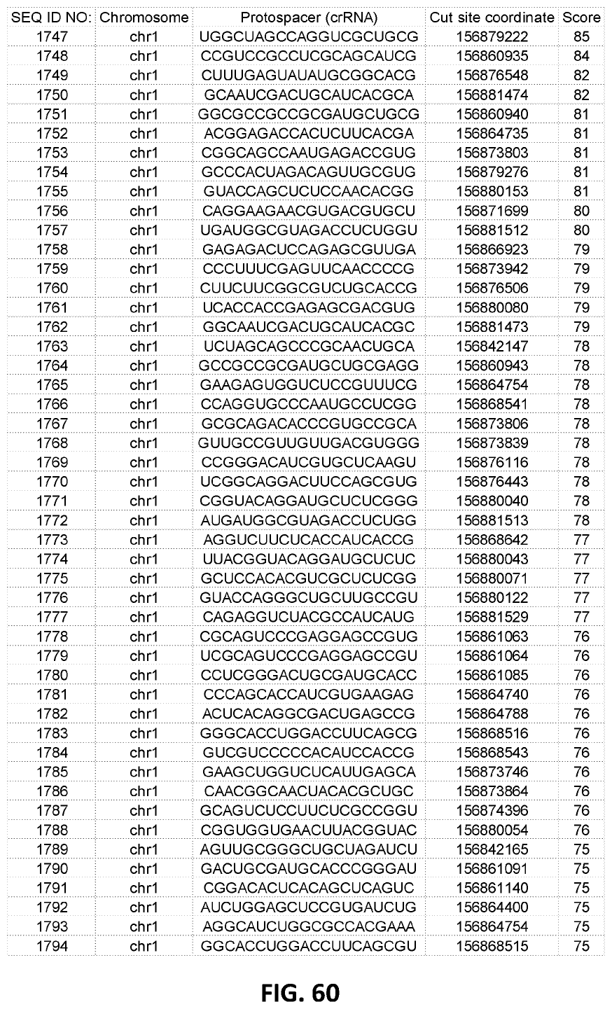

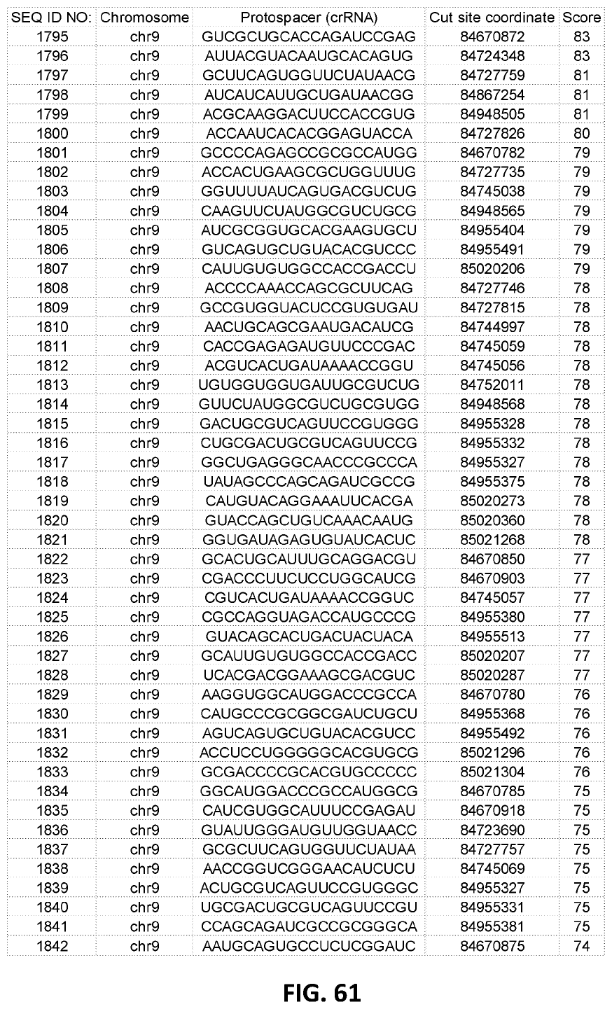

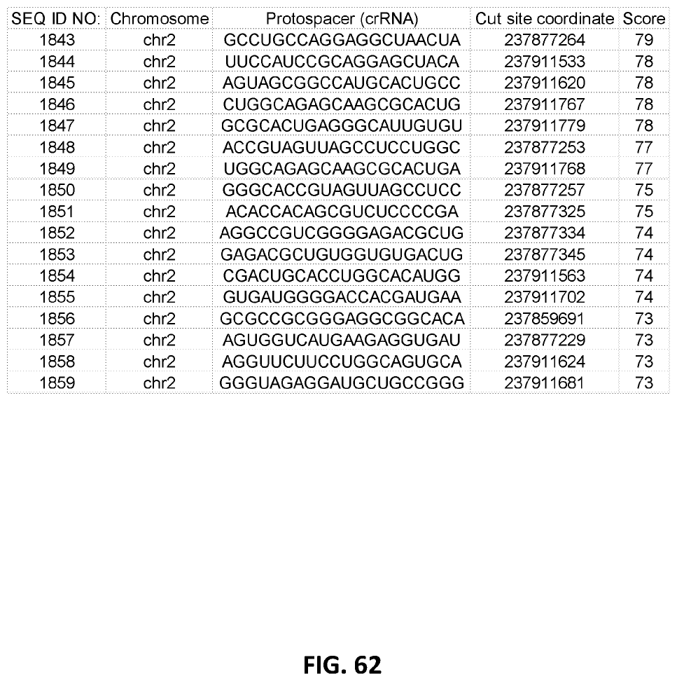

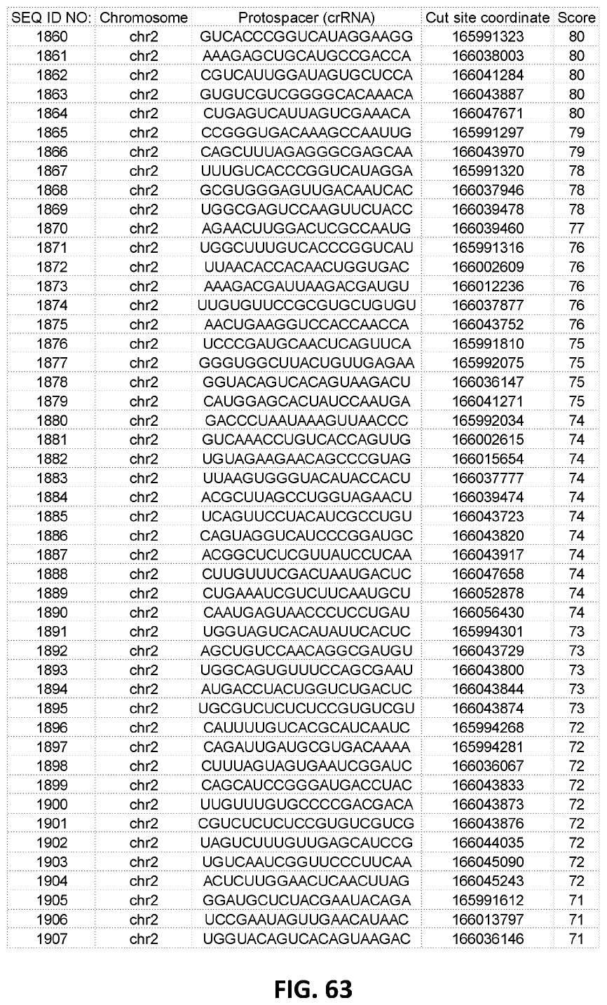

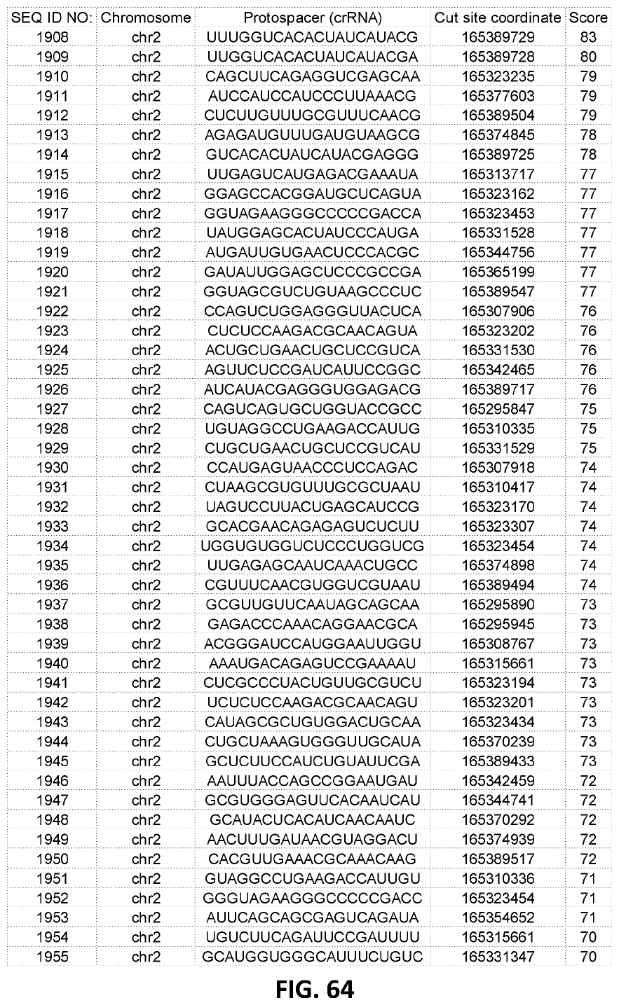

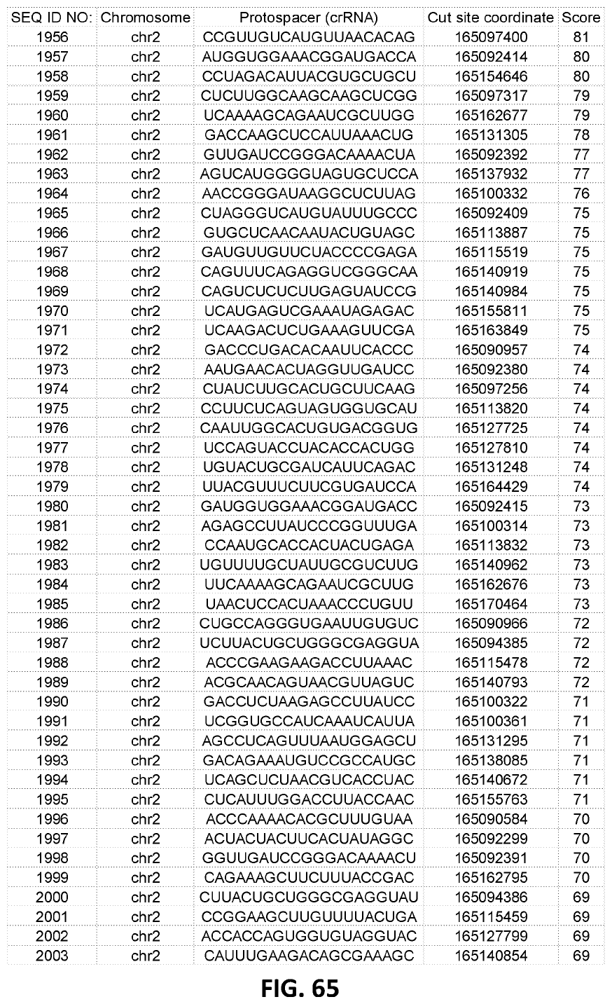

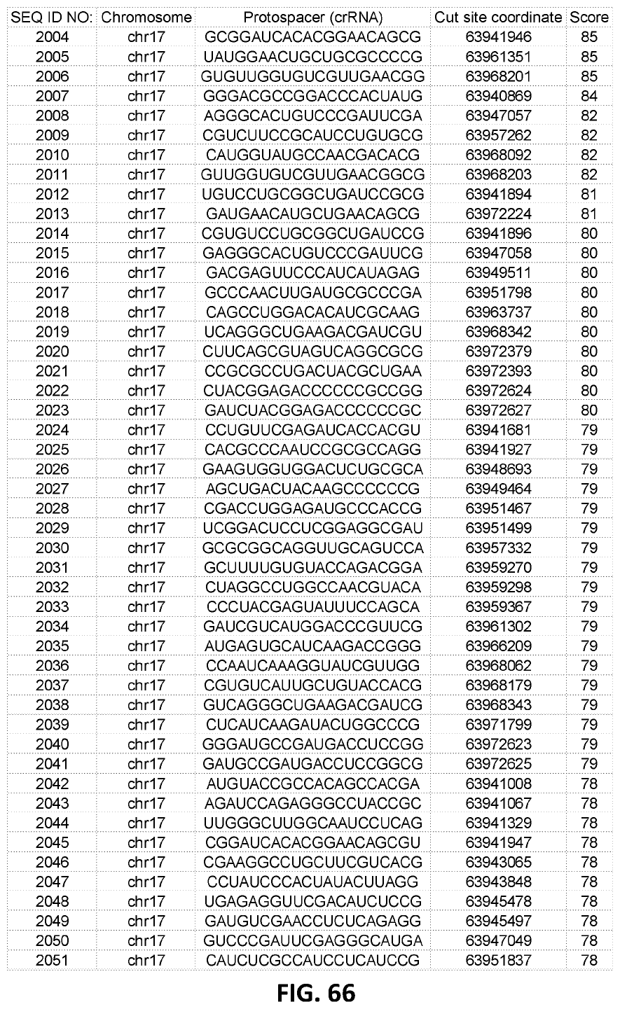

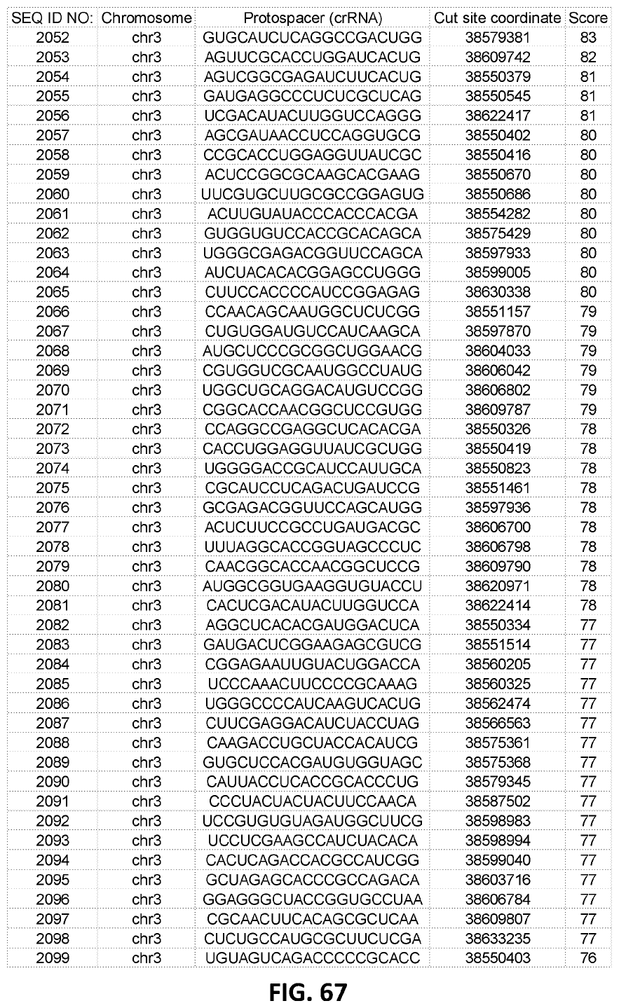

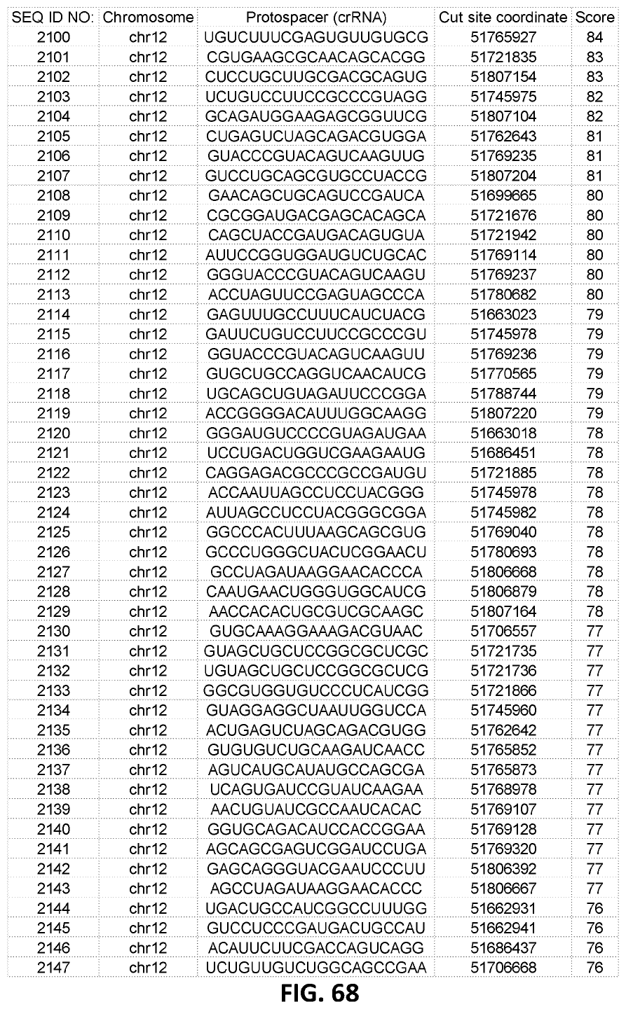

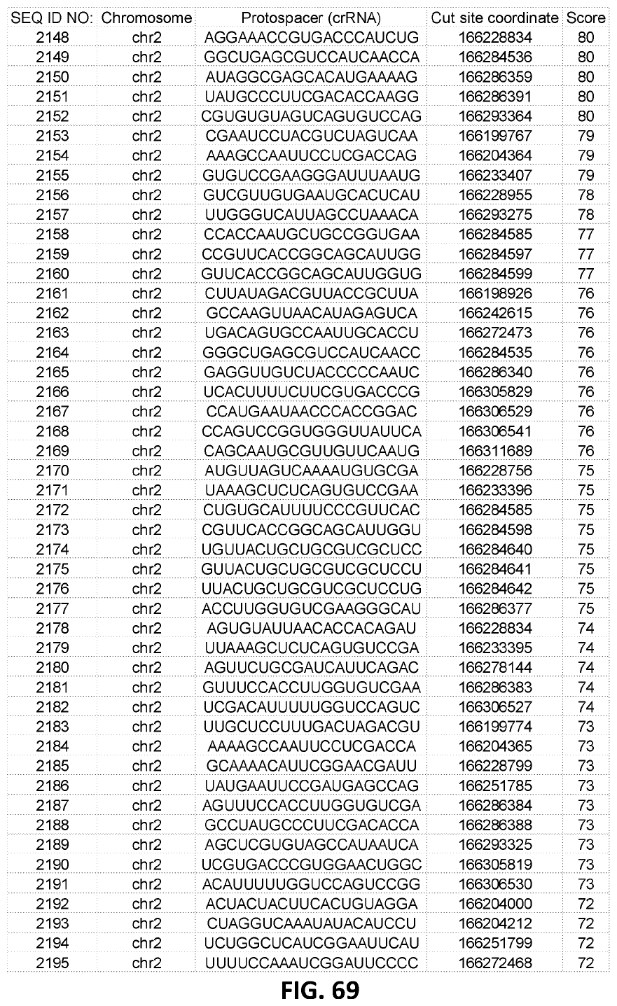

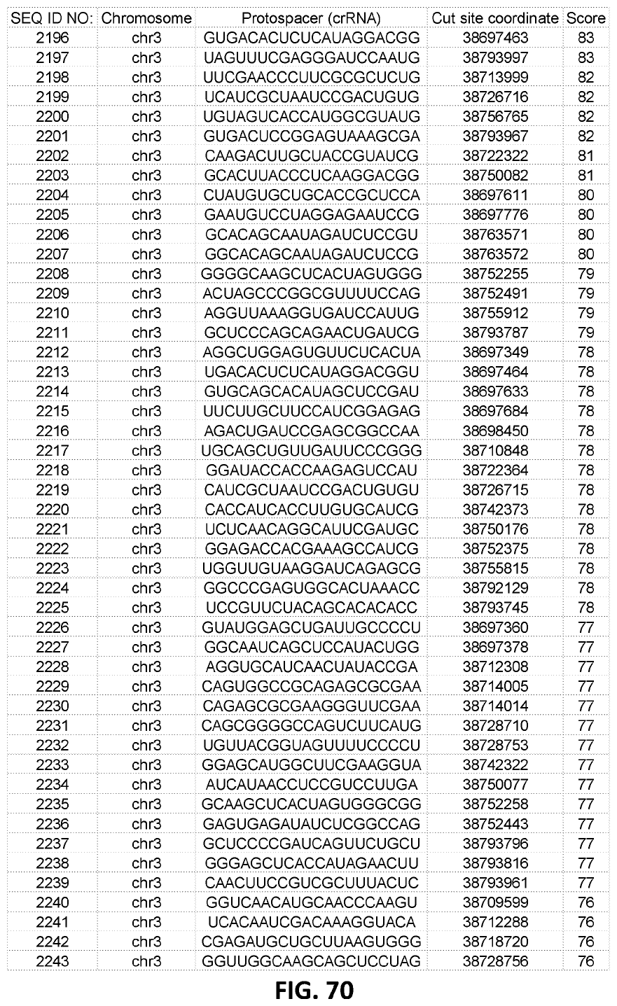

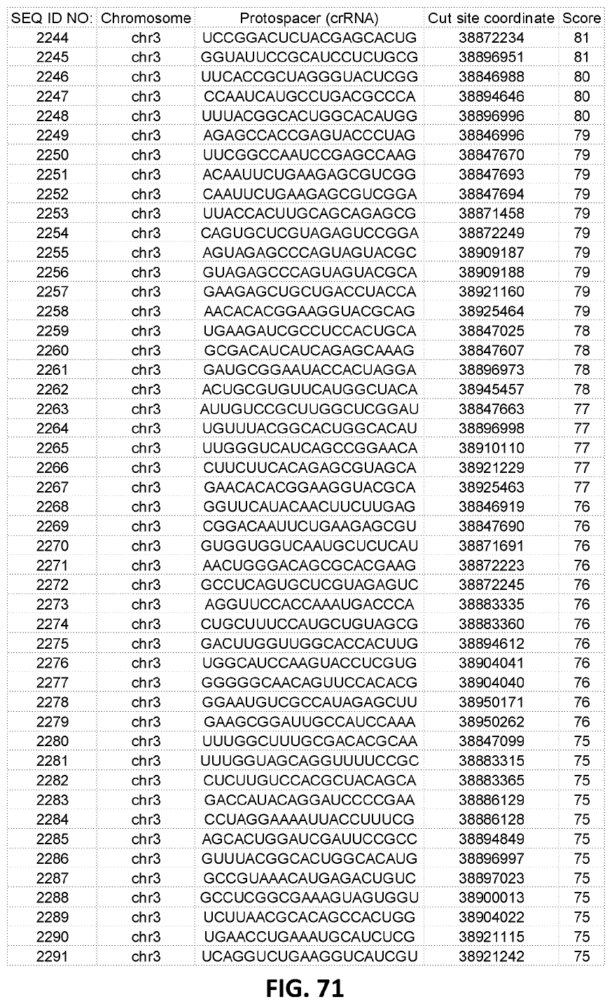

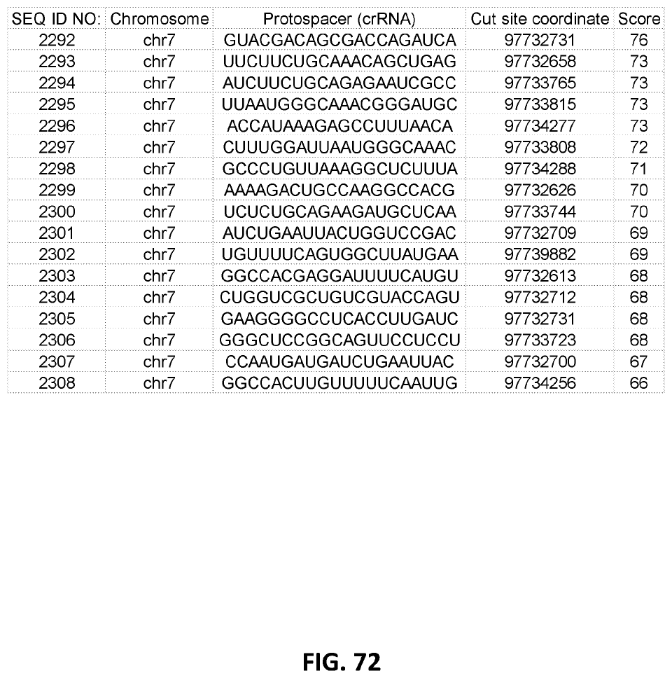

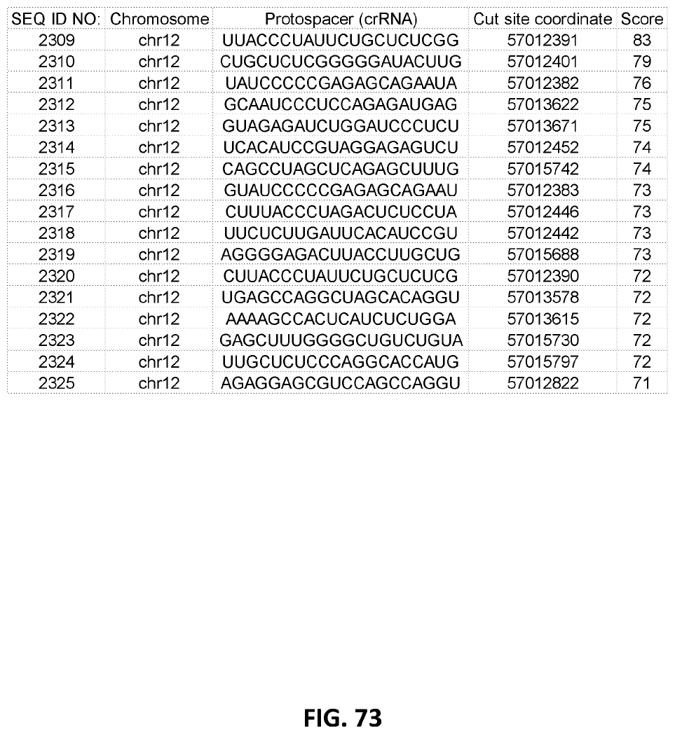

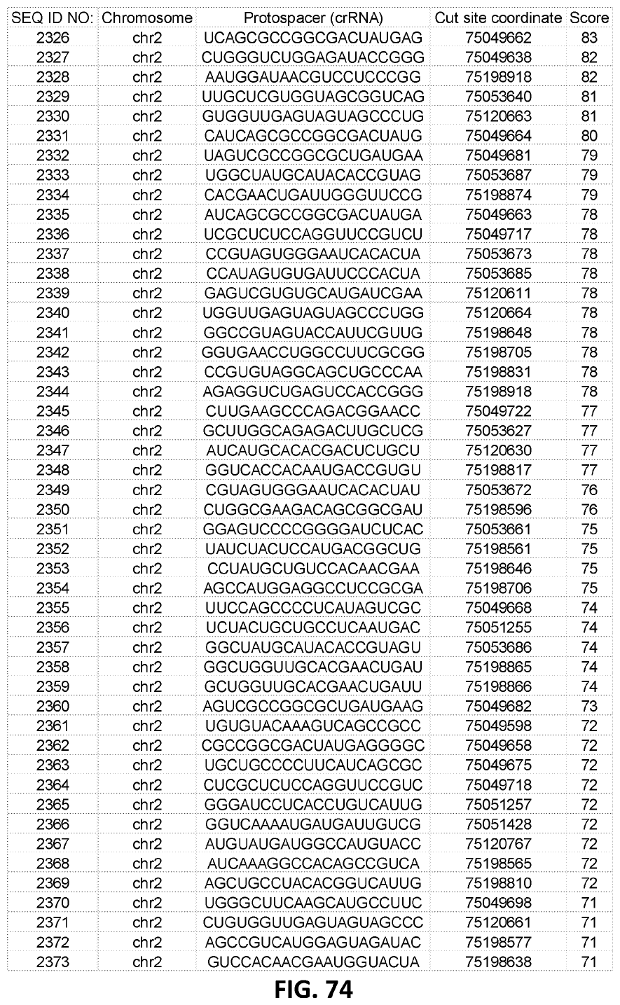

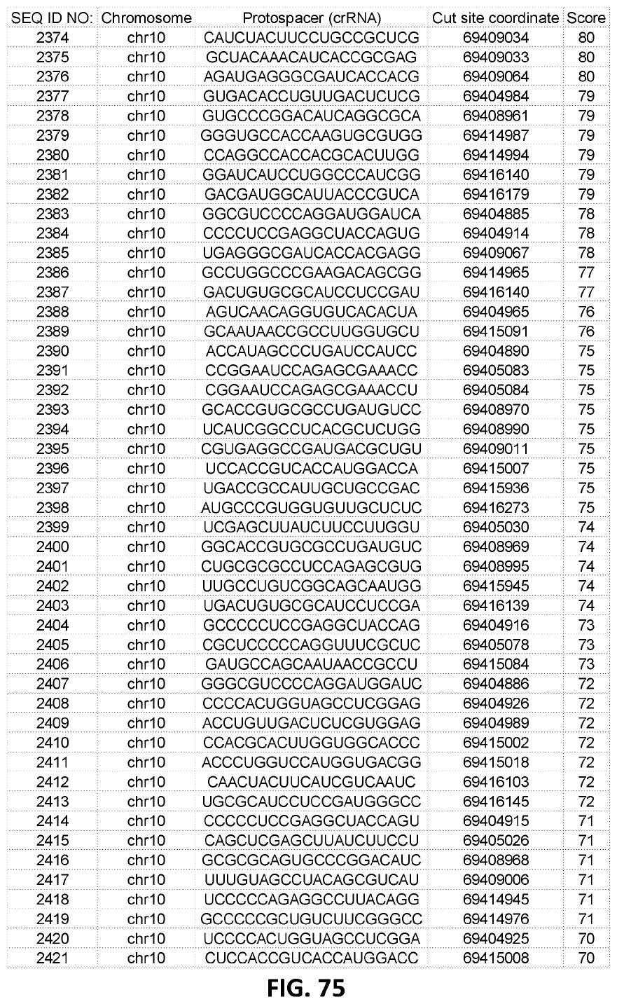

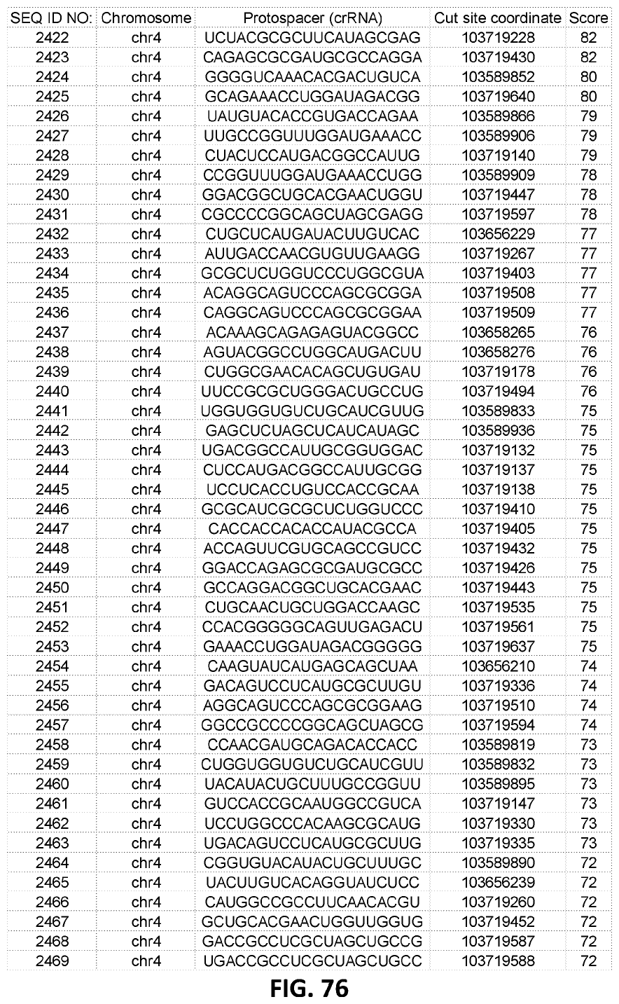

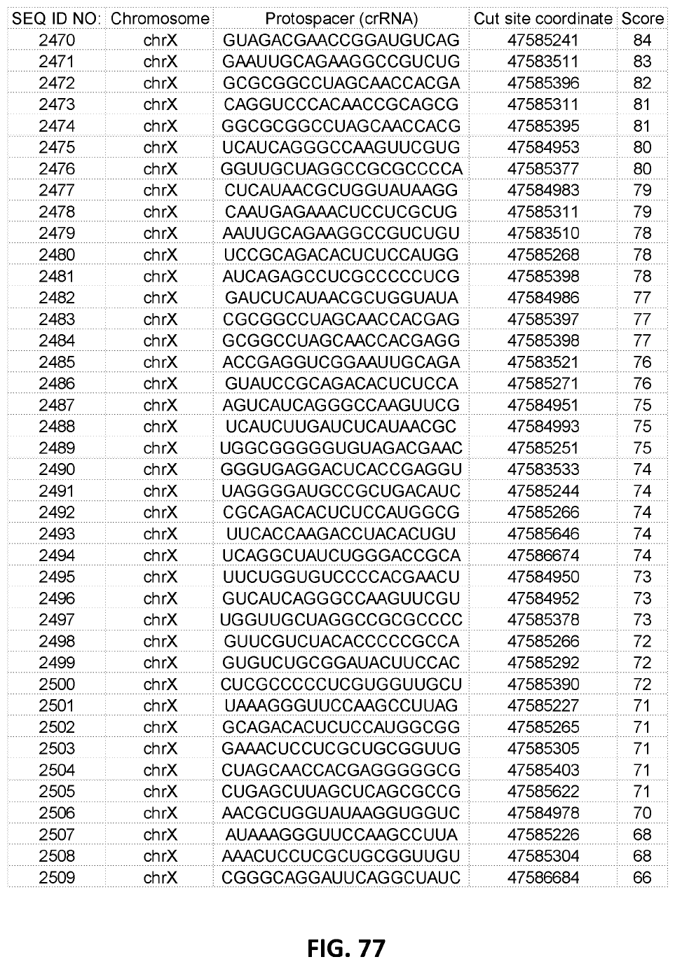

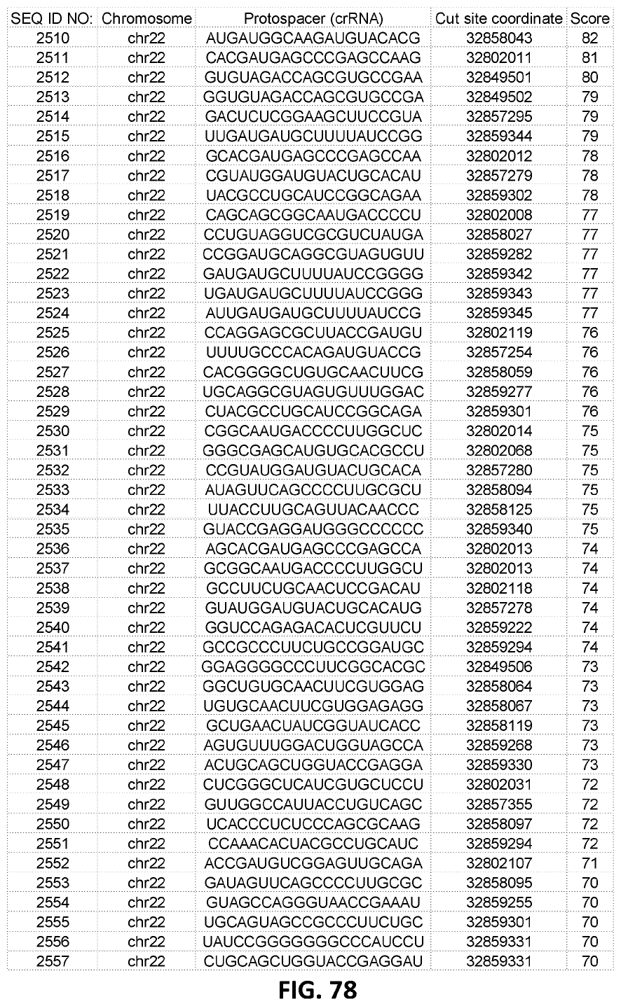

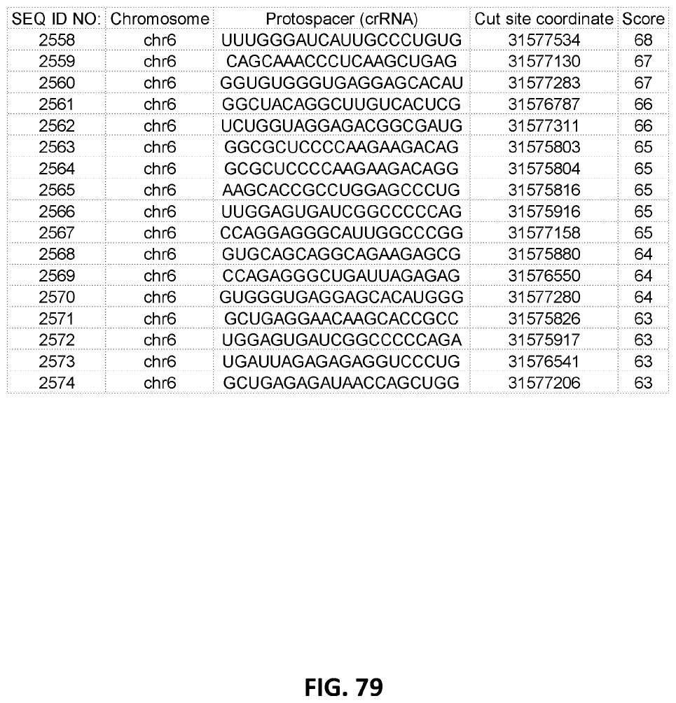

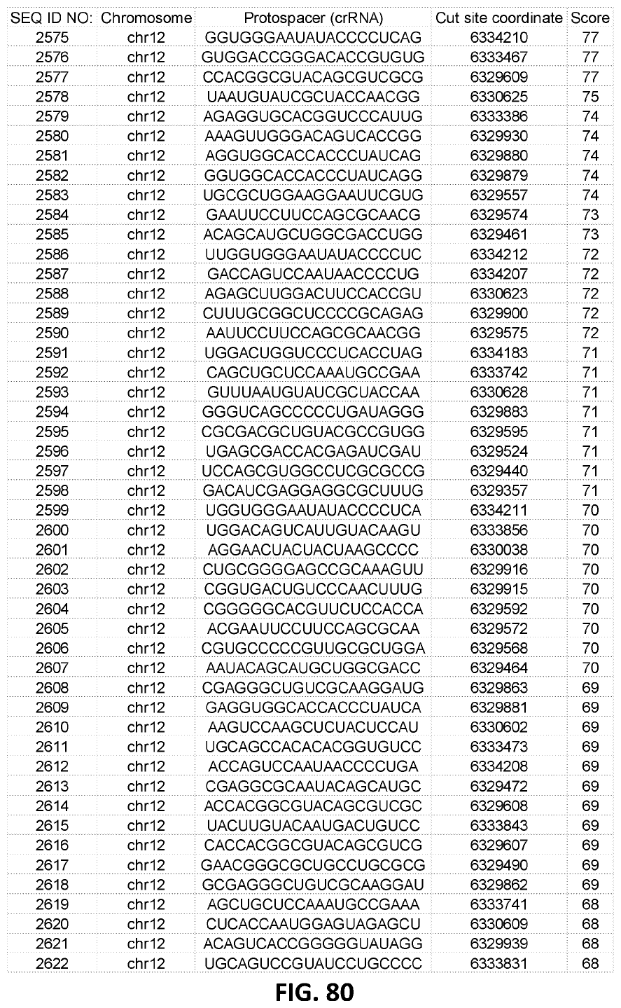

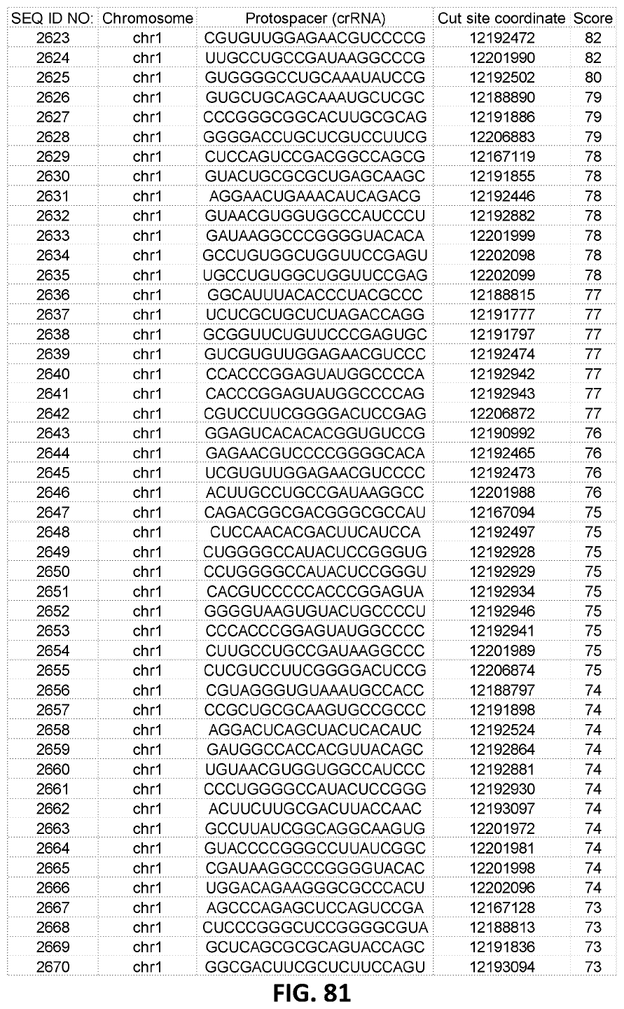

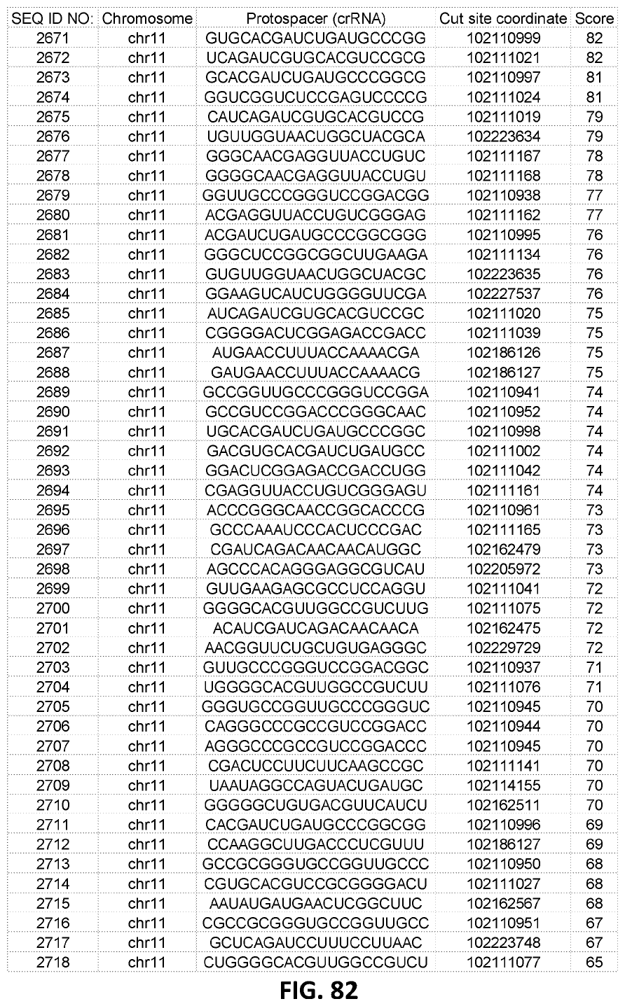

### DETAILED DESCRIPTION OF THE DISCLOSURE

### I. Introduction

Provided herein are compositions and methods for silencing the translation of one or more proteins in an animal in need thereof to treat a disease, illness or condition associated with pain.

In some embodiments, receptor signaling is silenced by CRISPR editing of the gene encoding the receptor. In some embodiments, the CRISPR editing results in ablation of a transmembrane domain (i.e., generation of a soluble decoy receptor). In some embodiments, the CRISPR editing results in ablation of a cytoplasmic domain (i.e., generation of a membrane-bound decoy receptor). In particular embodiments, compositions and methods are provided to gene-edit FGF2, CCN2, ADAMTS5, MMP1 and/or NGF.

### II. Definitions

Unless defined otherwise, all technical and scientific terms used herein have the same meaning as is commonly understood by one of skill in the art to which this invention belongs. All patents and publications referred to herein are incorporated by reference in their entireties.

In some embodiments, pain is ameliorated by silencing of a nociception signaling protein (or its cognate receptor) via CRISPR editing of the gene encoding the protein (or receptor). In some embodiments, the CRISPR editing results in ablation of a transmembrane domain of a pain receptor (i.e., generation of a soluble decoy receptor). In some embodiments, the CRISPR editing results in ablation of the cytoplasmic domain of a pain receptor (i.e., generation of a membrane-bound decoy receptor). In particular embodiments, compositions and methods are provided to gene-edit (i) one or more growth factors or growth factor receptors (e.g., FGF2, CCN2, NGF, NTF3, NTF4, BDNF, FGFR1, NGFR, NTRK1, or NTRK2), (ii) one or more metalloproteases or regulators thereof (e.g., ADAM17, ADAMTS1, ADAMTS5, MMP1, MMP2, MMP3, MMP7, MMP8, MMP10, MMP12, MMP13, TIMP1, or TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, or IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, or ATP1A1), (v) one or more other regulators of cell signaling (e.g., CALCA, CALCB, CALCRL, RAMP1, ADM, CRCP, YAP1, MRGPRX2), or (vi) a combination of any genes of (i)-(v) to ameliorate pain.

### Definitions

Unless defined otherwise, all technical and scientific terms used herein have the same meaning as is commonly understood by one of skill in the art to which this disclosure belongs. All patents and publications referred to herein are incorporated by reference in their entireties.

The term “FGF2 gene” refers to a mammalian gene encoding a Fibroblast growth factor 2 polypeptide. Non-limiting examples of FGF2 genes include: NCBI Gene ID: 2247 [human], NCBI Gene ID: 403857 [canine], NCBI Gene ID: 100033955 [equine], NCBI Gene ID: 100135772 [feline], as well as synonymous and non-synonymous sequence variants thereof. Non-limiting examples of gene products encoded by an FGF2 gene include: UniProt: P09038; NP_001348594.1 [human], XP_038421156.1 [canine], NP_001182150.1 [equine], XP_044911834.1 [feline]), as well as sequence variants, isoforms encoded by alternative splicing, and various glycoforms thereof. Canonically, the proteins encoded by the genes listed above act as ligands for the FGF receptors FGFR1, FGFR2, FGFR3 and FGFR4 in addition to strongly binding heparin and integrins. Additionally, FGF2 signaling is thought to impact localized nociception via at least its pro-angiogenic activity and has been implicated in pain perception related to at least IVD degeneration and at joint lesions. In some instances, and merely for the sake of disambiguation, a prefix is added when referring to the protein or gene of a particular species (with h, c, e, and f referring to the human, canine, equine, and feline forms, respectively).

The term “FGFR1 gene” refers to a mammalian gene encoding a Fibroblast Growth Factor Receptor 1 polypeptide. Non-limiting examples of FGFR1 genes include: NCBI Gene ID: 2260 [human], NCBI Gene ID: 100856477 [canine], NCBI Gene ID: 100057614 [equine], NCBI Gene ID: 101086055 [feline] as well as synonymous and non-synonymous sequence variants thereof. Non-limiting examples of gene products encoded by an FGFR1 gene include: UniProt: P11362; NP_001167534.1 [human], XP_038545782.1 [canine], XP_023486323.1 [equine], XP_011279822.1 [feline], as well as sequence variants, isoforms encoded by alternative splicing, and various glycoforms thereof. Canonically, the proteins encoded by the genes listed above are tyrosine-protein kinases that act as cell-surface receptor for fibroblast growth factors. In that role, they play an essential role in the regulation of embryonic development, cell proliferation, differentiation and migration. In some instances, and merely for the sake of disambiguation, a prefix is added when referring to the protein or gene of a particular species (with h, c, e, and f referring to the human, canine, equine, and feline forms, respectively).

The term “CCN2 gene refers to a mammalian gene encoding a Cellular Communication Network Factor 2 polypeptide. Non-limiting examples of CCN2 genes include: NCBI Gene ID: 1490 [human], NCBI Gene ID: 476202 [canine], NCBI Gene ID: 100073098 [equine], NCBI Gene ID: 101094598 [feline], as well as synonymous and non-synonymous sequence variants thereof. Non-limiting examples of gene products encoded by a CCN2 gene include: UniProt: P29279; NP_001892.2 [human], XP_038321343.1 [canine], XP_023506869.1 [equine], XP_023110145.1 [feline], as well as sequence variants, isoforms encoded by alternative splicing, and various glycoforms thereof. Canonically, the proteins encoded by the genes listed above are mitogens secreted by vascular endothelial cells and are related to chondrocyte proliferation and differentiation, cell adhesion in many cell types. In some instances, and merely for the sake of disambiguation, a prefix is added when referring to the protein or gene of a particular species (with h, c, e, and f referring to the human, canine, equine, and feline forms, respectively).

The term “ADAMTS5 gene” refers to a mammalian gene encoding an ADAM Metallopeptidase with Thrombospondin Type 1 Motif 5 polypeptide. Non-limiting examples of ADAMTS5 genes include: NCBI Gene ID: 11096 [human], NCBI Gene ID: 487713 [canine], NCBI Gene ID: 100066005 [equine], NCBI Gene ID: 101085063 [feline], as well as synonymous and non-synonymous sequence variants thereof. Non-limiting examples of gene products encoded by an ADAMTS5 gene include: UniProt: Q9UNA0; NP_008969.2 [human], XP_038299214.1 [canine], XP 023485737.1 [equine], XP_023094603.1 [feline], as well as sequence variants, isoforms encoded by alternative splicing, and various glycoforms thereof. Canonically, members of the family share several distinct protein modules, including a propeptide region, a metalloproteinase domain, a disintegrin-like domain, and a thrombospondin type 1 (TS) motif with individual members of the family differing in the number of C-terminal TS motifs. ADAMTS5 has two unique C-terminal domains. Once proteolytically processed to generate the mature enzyme, ADAMTS5 functions as an aggrecanase that cleaves aggrecan, a major proteoglycan of cartilage, and may mediate cartilage destruction in osteoarthritis. In some instances, and merely for the sake of disambiguation, a prefix is added when referring to the protein or gene of a particular species (with h, c, e, and f referring to the human, canine, equine, and feline forms, respectively).

The term “ADAMTS1 gene” refer to a mammalian gene encoding an ADAM Metallopeptidase with Thrombospondin Type 1 Motif 1 polypeptide. Non-limiting examples of ADAMTS1 genes include: NCBI Gene ID: 9510 [human], NCBI Gene ID: 100686153 [canine], NCBI Gene ID: 791251 [equine], NCBI Gene ID: 101085309 [feline], as well as synonymous and non-synonymous sequence variants thereof. Non-limiting examples of gene products encoded by an ADAMTS1 gene include: UniProt: Q9UH18; NP_008919.3 [human], XP_038374156.1 [canine], XP_023485736.1 [equine], XP_019695041.3 [feline], as well as sequence variants, isoforms encoded by alternative splicing, and various glycoforms thereof. Canonically, members of the family share several distinct protein modules, including a propeptide region, a metalloproteinase domain, a disintegrin-like domain, and a thrombospondin type 1 (TS) motif with individual members of the family differing in the number of C-terminal TS motifs. ADAMTS1 contains two disintegrin loops and three C-terminal TS motifs. The protein has anti-angiogenic activity and functions as an aggrecanase that cleaves aggrecan, a major proteoglycan of cartilage, and may be involved in its turnover and has been associated with various inflammatory processes. In some instances, and merely for the sake of disambiguation, a prefix is added when referring to the protein or gene of a particular species (with h, c, e, and f, referring to the human, canine, equine, and feline forms, respectively).

The term “MMP1 gene” refers to a mammalian gene encoding a Matrix Metallopeptidase 1 polypeptide. Non-limiting examples of MMP1 genes include: NCBI Gene ID: 4312 [human], NCBI Gene ID: 489428 [canine], NCBI Gene ID: 100033896 [equine], NCBI Gene ID: 101084217 [feline], as well as synonymous and non-synonymous sequence variants thereof. Non-limiting examples of gene products encoded by an MMP1 gene include: UniProt: P03956; NP_001139410.1 [human], XP_038521018.1 [canine], NP_001075316.1 [equine], XP 003992365.2 [feline], as well as sequence variants, isoforms encoded by alternative splicing, and various glycoforms thereof. Canonically, MMP1 is proteolytically processed from a preproprotein to generate the mature protease. This secreted protease breaks down the interstitial collagens, including types I, II, and III. In some instances, and merely for the sake of disambiguation, a prefix is added when referring to the protein or gene of a particular species (with h, c, e, and f, referring to the human, canine, equine, and feline forms, respectively).

The term “MMP2 gene” refers to a mammalian gene encoding a Matrix Metallopeptidase 2 polypeptide. Non-limiting examples of MMP2 genes include: NCBI Gene ID: 4313 [human], NCBI Gene ID: 403733 [canine], NCBI Gene ID: 100033948 [equine], NCBI Gene ID: 101098838 [feline], as well as synonymous and non-synonymous sequence variants thereof. Non-limiting examples of gene products encoded by an MMP2 gene include: UniProt: P08253; NP_001121363.1 [human], XP_038515255.1 [canine], XP_023492775.1 [equine], XP_003998091.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP2 is a gelatinase A, type IV collagenase, that contains three fibronectin type II repeats in its catalytic site that allow binding of denatured type IV and V collagen and elastin. In some instances, and merely for the sake of disambiguation, a prefix is added when referring to the protein or gene of a particular species (with h, c, e, and f, referring to the human, canine, equine, and feline forms, respectively).

The term “MMP3 gene” refers to a mammalian gene encoding a Matrix Metallopeptidase 3 polypeptide. Non-limiting examples of MMP3 genes include: NCBI Gene ID: 4314 [human], NCBI Gene ID: 403733 [canine], NCBI Gene ID: 100034195 [equine], NCBI Gene ID: 493666 [feline], as well as synonymous and non-synonymous sequence variants thereof. Non-limiting examples of gene products encoded by an MMP3 gene include: UniProt: P08254; NP_002413.1 [human], NP_001002967.1 [canine], NP_001075964.1 [equine], XP_003992356.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP3 is an enzyme that degrades fibronectin, laminin, collagens III, IV, IX, and X, and cartilage proteoglycans and is thought to be involved in wound repair, progression of atherosclerosis, and tumor initiation. In some instances, and merely for the sake of disambiguation, a prefix is added when referring to the protein or gene of a particular species (with h, c, e, and f referring to the human, canine, equine, and feline forms, respectively).

The term “MMP7 gene” refers to a mammalian gene encoding a Matrix Metallopeptidase 7 polypeptide. Non-limiting examples of MMP7 genes include: NCBI Gene ID: 4316 [human], NCBI Gene ID: 489432 [canine], NCBI Gene ID: 100068985 [equine], NCBI Gene ID: 727698 [feline], as well as synonymous and non-synonymous sequence variants thereof. Non-limiting examples of gene products encoded by an MMP7 gene include: UniProt: P09237; NP_002414.1 [human], NP_001229655.1 [canine], XP_001498859.1 [equine], XP_003992352.1 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP7 is proteolytically processed to generate the mature protease, which breaks down proteoglycans, fibronectin, elastin and casein in addition to activating procollagenase. In some instances, and merely for the sake of disambiguation, a prefix is added when referring to the protein or gene of a particular species (with h, c, e, and f, referring to the human, canine, equine, and feline forms, respectively).

The term “MMP8 gene” refers to a mammalian gene encoding a Matrix Metallopeptidase 8 polypeptide. Non-limiting examples of MMP8 genes include: NCBI Gene ID: 4317 [human], NCBI Gene ID: 489429 [canine], NCBI Gene ID: 100069005 [equine], NCBI Gene ID: 101080995 [feline], as well as synonymous and non-synonymous sequence variants thereof. Non-limiting examples of gene products encoded by an MMP8 gene include: UniProt: P22894; NP_001291370.1 [human], XP_038521019.1 [canine], XP_005611595.1 [equine], XP_003992354.3 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP8 is an enzyme that degrades interstitial collagens. In some instances, and merely for the sake of disambiguation, a prefix is added when referring to the protein or gene of a particular species (with h, c, e, and f, referring to the human, canine, equine, and feline forms, respectively).

The term “MMP10 gene” refers to a mammalian gene encoding a Matrix Metallopeptidase 10 polypeptide. Non-limiting examples of MMP10 genes include: NCBI Gene ID: 4319 [human], NCBI Gene ID: 100146442 [equine], NCBI Gene ID: 101081247 [feline], as well as synonymous and non-synonymous sequence variants thereof. Non-limiting examples of gene products encoded by an MMP10 gene include: UniProt: P09238; NP_002416.1 [human], XP_005614947.1 [equine], XP_003992355.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP10 is an enzyme that degrades fibronectin, and type I, III, IV, and V gelatins. In some instances, and merely for the sake of disambiguation, a prefix is added when referring to the protein or gene of a particular species (with h, c, e, and f, referring to the human, canine, equine, and feline forms, respectively).

The term “MMP12 gene” refers to a mammalian gene encoding a Matrix Metallopeptidase 12 polypeptide. Non-limiting examples of MMP12 genes include: NCBI Gene ID: 4321 [human], NCBI Gene ID: 611789 [canine], NCBI Gene ID: 100069047 [equine], NCBI Gene ID: 101084472 [feline], as well as synonymous and non-synonymous sequence variants thereof. Non-limiting examples of gene products encoded by an MMP12 gene include: UniProt: P39900; NP_002417.2 [human], NP_001274067.1 [canine], XP_001498924.2 [equine], XP_003992366.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP12 is an enzyme with significant elastolytic activity and may be involved in tissue injury and remodeling. In some instances, and merely for the sake of disambiguation, a prefix is added when referring to the protein or gene of a particular species (with h, c, e, and f, referring to the human, canine, equine, and feline forms, respectively).

The term “MMP13 gene” refers to a mammalian gene encoding a Matrix Metallopeptidase 13 polypeptide. Non-limiting examples of MMP13 genes include: NCBI Gene ID: 4322 [human], NCBI Gene ID: 403763 [canine], NCBI Gene ID: 100009711 [equine], NCBI Gene ID: 493679 [feline], as well as synonymous and non-synonymous sequence variants thereof. Non-limiting examples of gene products encoded by an MMP13 gene include: UniProt: P45452; NP_002418.1 [human], XP_038521017.1 [canine], NP_001075273.1 [equine], XP_023094811.2 [feline], as well as sequence variants, isoforms encoded by alternative splicing, and various glycoforms thereof. Canonically, the protein encoded by this gene belongs to the broader family of zinc-dependent enzymes that cleave components of the extracellular matrix. MMP13 is an enzyme that degrades various types of collagen and has been implicated in wound healing, tissue remodeling, cartilage degradation, bone development, bone mineralization and ossification. In some instances, and merely for the sake of disambiguation, a prefix is added when referring to the protein or gene of a particular species (with h, c, e, and f, referring to the human, canine, equine, and feline forms, respectively).

The term “TIMP1 gene” refers to a mammalian gene encoding a TIMP Metallopeptidase Inhibitor 1 polypeptide. Non-limiting examples of TIMP1 genes include: NCBI Gene ID: 7076 [human], NCBI Gene ID: 403816 [canine], NCBI Gene ID: 100034220 [equine], NCBI Gene ID: 101095886 [feline], as well as synonymous and non-synonymous sequence variants thereof. Non-limiting examples of gene products encoded by a TIMP1 gene include: UniProt: P01033; NP_003245.1 [human], NP_001003182.1 [canine], XP_023488949.1 [equine], XP_023105059.1 [feline], as well as sequence variants, isoforms encoded by alternative splicing, and various glycoforms thereof. Canonically, the protein encoded by this gene functions by forming one to one complexes with target metalloproteinases, such as collagenases, irreversibly inactivating through binding to their catalytic zinc cofactor. TIMP1 acts on MMP1, MMP2, MMP3, MMP7, MMP8, MMP9, MMP10, MMP11, MMP12, MMP13 and MMP16, but not on MMP14 and has been shown to act as a growth factor regulating cell differentiation, migration and cell death. In some instances, and merely for the sake of disambiguation, a prefix is added when referring to the protein or gene of a particular species (with h, c, e, and f, referring to the human, canine, equine, and feline forms, respectively).

The term “TIMP3 gene” refers to a mammalian gene encoding a TIMP Metallopeptidase Inhibitor 3 polypeptide. Non-limiting examples of TIMP3 genes include: NCBI Gene ID: 7078 [human], NCBI Gene ID: 481289 [canine], NCBI Gene ID: 100033947 [equine], NCBI Gene ID: 101091215 [feline], as well as synonymous and non-synonymous sequence variants thereof. Non-limiting examples of gene products encoded by a TIMP3 gene include: UniProt: P35625; NP_000353.1 [human], NP_001271368.1 [canine], NP_001075339.1 [equine], XP_003989265.1 [feline], as well as sequence variants, isoforms encoded by alternative splicing, and various glycoforms thereof. Canonically, the protein encoded by this gene complexes with metalloproteinases (such as collagenases) to irreversibly inactivate them by binding to their catalytic zinc cofactor. TIMP3 is known to act on MMP1, MMP2, MMP3, MMP7, MMP9, MMP13, MMP14 and MMP15. In some instances, and merely for the sake of disambiguation, a prefix is added when referring to the protein or gene of a particular species (with h, c, e, and f referring to the human, canine, equine, and feline forms, respectively).

The term “CXCL1 gene” refers to a mammalian gene encoding a C-X-C Motif Chemokine Ligand 1 polypeptide. Non-limiting examples of CXCL1 genes include: NCBI Gene ID: 2919 [human], NCBI Gene ID: 100034121 [equine], NCBI Gene ID: 102901432 [feline], as well as synonymous and non-synonymous sequence variants thereof. Non-limiting examples of gene products encoded by a CXCL1 gene include: UniProt: P09341; NP_001502.1 [human], NP_001296409.1 [equine], XP_023108817.2 [feline], as well as sequence variants, isoforms encoded by alternative splicing, and various glycoforms thereof. Canonically, the protein encoded by this gene has chemotactic activity for neutrophils and may play a role inflammation. In some instances, and merely for the sake of disambiguation, a prefix is added when referring to the protein or gene of a particular species (with h, c, e, and f referring to the human, canine, equine, and feline forms, respectively).

The term “CXCL2 gene” refers to a mammalian gene encoding a C-X-C Motif Chemokine Ligand 2 polypeptide. Non-limiting examples of CXCL2 genes include: NCBI Gene ID: 2920 [human], NCBI Gene ID: 100233237 [equine], as well as synonymous and non-synonymous sequence variants thereof. Non-limiting examples of gene products encoded by a CXCL2 gene include: UniProt: P19875, Q9UPB8; NP_002080.1 [human], NP_001137427.1 [equine], as well as sequence variants, isoforms encoded by alternative splicing, and various glycoforms thereof. Canonically, the protein encoded by this gene has antimicrobial function via its regulation of inflammatory and immunoregulatory processes. CXCL2 is expressed at the site of inflammation and has been shown to suppress proliferation of hematopoietic progenitor cells. In some instances, and merely for the sake of disambiguation, a prefix is added when referring to the protein or gene of a particular species (with h, c, e, and f referring to the human, canine, equine, and feline forms, respectively).

The term “CXCL3 gene” refers to a mammalian gene encoding a C-X-C Motif Chemokine Ligand 3 polypeptide. Non-limiting examples of CXCL3 genes include: NCBI Gene ID: 2921 [human] NCBI Gene ID: 100056258 [equine], as well as synonymous and non-synonymous sequence variants thereof. Non-limiting examples of gene products encoded by a CXCL3 gene include: UniProt: P19876, Q4W5H9; NP_002081.2 [human], NP_001137265.1 [equine], as well as sequence variants, isoforms encoded by alternative splicing, and various glycoforms thereof. Canonically, the protein encoded by this gene is a secreted growth factor that signals through the G-protein coupled receptor, CXCR2 and plays a role in inflammation and as a chemoattractant for neutrophils. In some instances, and merely for the sake of disambiguation, a prefix is added when referring to the protein or gene of a particular species (with h, c, e, and f referring to the human, canine, equine, and feline forms, respectively).

The term “CXCL5 gene” refers to a mammalian gene encoding a C-X-C Motif Chemokine Ligand 5 polypeptide. Non-limiting examples of CXCL5 genes include: NCBI Gene ID: 6374 [human], as well as synonymous and non-synonymous sequence variants thereof. Non-limiting examples of gene products encoded by a CXCL5 gene include: UniProt: P19876, Q4W5H9; NP_002081.2 [human], UniProt: P97885 [rat], UniProt: P50228 [mouse] as well as sequence variants, isoforms encoded by alternative splicing, and various glycoforms thereof. Canonically, the protein encoded by this gene is thought to interact with the G-protein coupled receptor, CXCR2 to promote angiogenesis, remodel connective tissues and recruit neutrophils. In some instances, and merely for the sake of disambiguation, a prefix is added when referring to the protein or gene of a particular species (with h, c, e, and f referring to the human, canine, equine, and feline forms, respectively).

The term “CXCL6 gene” refers to a mammalian gene encoding a C-X-C Motif Chemokine Ligand 6 polypeptide. Non-limiting examples of CXCL6 genes include: NCBI Gene ID: 6372 [human], NCBI Gene ID: 106557449 [canine], NCBI Gene ID: 100033988 [equine], NCBI Gene ID: 101094593 [feline], as well as synonymous and non-synonymous sequence variants thereof. Non-limiting examples of gene products encoded by a CXCL6 gene include: UniProt: P80162; NP_002984.1 [human], XP_038541813.1 [canine], NP_001075355.2 [equine], XP_003985379.3 [feline] as well as sequence variants, isoforms encoded by alternative splicing, and various glycoforms thereof. Canonically, the protein encoded by this gene is a chemotactic factor for neutrophils and exhibits antibacterial activity. In some instances, and merely for the sake of disambiguation, a prefix is added when referring to the protein or gene of a particular species (with h, c, e, and f referring to the human, canine, equine, and feline forms, respectively).

The term “CXCL8 gene” refers to a mammalian gene encoding a C-X-C Motif Chemokine Ligand 8 polypeptide. Non-limiting examples of CXCL8 genes include: NCBI Gene ID: 3576 [human], NCBI Gene ID: 403850 [canine], NCBI Gene ID: 100037400 [equine], NCBI Gene ID: 493836 [feline], as well as synonymous and non-synonymous sequence variants thereof. Non-limiting examples of gene products encoded by a CXCL8 gene include: UniProt: P10145; NP_000575.1 [human], NP_001003200.1 [canine], NP_001077420.2 [equine], NP_001009281.1 [feline], as well as sequence variants, isoforms encoded by alternative splicing, and various glycoforms thereof. Canonically, the protein encoded by this gene is secreted by mononuclear macrophages, neutrophils, eosinophils, T lymphocytes, epithelial cells, and fibroblasts and functions as a chemotactic factor that guides neutrophils to the site of infection. CXCL8 also participates with other cytokines in the proinflammatory signaling cascade. In some instances, and merely for the sake of disambiguation, a prefix is added when referring to the protein or gene of a particular species (with h, c, e, and f referring to the human, canine, equine, and feline forms, respectively).

The term “CCL2 gene” refers to a mammalian gene encoding a C-C Motif Chemokine Ligand 2” polypeptide. Non-limiting examples of CCL2 genes include: NCBI Gene ID: 6347 [human], NCBI Gene ID: 403981 [canine], NCBI Gene ID: 100034136 [equine], NCBI Gene ID: 100127112 [feline], as well as synonymous and non-synonymous sequence variants thereof. Non-limiting examples of gene products encoded by a CCL2 gene include: UniProt: P13500; NP_002973.1 [human], NP_001003297.1 [canine], NP_001075400.1 [equine], XP_003996605.1 [feline], as well as sequence variants, isoforms encoded by alternative splicing, and various glycoforms thereof. Canonically, the protein encoded by this gene acts as a ligand for CCR2, which induces chemotactic activity for monocytes and basophils (but not neutrophils or eosinophils). In some instances, and merely for the sake of disambiguation, a prefix is added when referring to the protein or gene of a particular species (with h, c, e, and f, referring to the human, canine, equine, and feline forms, respectively).

The term “CCL3 gene” refers to a mammalian gene encoding a C-C Motif Chemokine Ligand 3 polypeptide. Non-limiting examples of CCL3 genes include: NCBI Gene ID: 6348 [human], NCBI Gene ID: 448787 [canine], NCBI Gene ID: 100057909 [equine], NCBI Gene ID: 100302540 [feline], as well as synonymous and non-synonymous sequence variants thereof. Non-limiting examples of gene products encoded by a CCL3 gene include: UniProt: P10147; NP_002974.1 [human], NP_001005251.2 [canine], NP_001108413.1 [equine], NP_001157129.1 [feline], as well as sequence variants, isoforms encoded by alternative splicing, and various glycoforms thereof. Canonically, the protein encoded by this gene plays a role in inflammatory responses through binding to the receptors CCR1, CCR4 and CCR5. In some instances, and merely for the sake of disambiguation, a prefix is added when referring to the protein or gene of a particular species (with h, c, e, and f, referring to the human, canine, equine, and feline forms, respectively).

The term “CCL5 gene” refers to a mammalian gene encoding a C-C Motif Chemokine Ligand 5 polypeptide. Non-limiting examples of CCL5 genes include: NCBI Gene ID: 6352 [human], NCBI Gene ID: 403522 [canine], NCBI Gene ID: 100033925 [equine], NCBI Gene ID: 493689 [feline], as well as synonymous and non-synonymous sequence variants thereof. Non-limiting examples of gene products encoded by a CCL5 gene include: UniProt: P13501; NP_001265665.1 [human], NP_001003010.1 [canine], NP_001075332.1 [equine], NP_001009827.1 [feline]) as well as sequence variants, isoforms encoded by alternative splicing, and various glycoforms thereof. Canonically, the protein encoded by this gene functions as a chemoattractant for blood monocytes, memory T helper cells and eosinophils, induces the release of histamine from basophils, and activates eosinophils. In some instances, and merely for the sake of disambiguation, a prefix is added when referring to the protein or gene of a particular species (with h, c, e, and f, referring to the human, canine, equine, and feline forms, respectively).

The term “CCL7 gene” refers to a mammalian gene encoding a C-C Motif Chemokine Ligand 7 polypeptide. Non-limiting examples of CCL7 genes include: NCBI Gene ID: 6354 [human], NCBI Gene ID: 491148 [canine], NCBI Gene ID: 100071714 [equine], NCBI Gene ID: 101096931 [feline], as well as synonymous and non-synonymous sequence variants thereof. Non-limiting examples of gene products encoded by a CCL7 gene include: UniProt: P80098; NP_006264.2 [human], NP_001010960.1 [canine], XP_005597638.1 [equine], XP_044900774.1 [feline], as well as sequence variants, isoforms encoded by alternative splicing, and various glycoforms thereof. Canonically, the protein encoded by this gene is a secreted chemokine which attracts macrophages during inflammation and metastasis and is an in vivo substrate of MMP2. In some instances, and merely for the sake of disambiguation, a prefix is added when referring to the protein or gene of a particular species (with h, c, e, and f, referring to the human, canine, equine, and feline forms, respectively).

The term “CCL20 gene” refers to a mammalian gene encoding a C-C Motif Chemokine Ligand 20 polypeptide. Non-limiting examples of CCL20 genes include: NCBI Gene ID: 6364 [human], NCBI Gene ID: 448790 [canine], NCBI Gene ID: 100629808 [equine], NCBI Gene ID: 101089032 [feline], as well as synonymous and non-synonymous sequence variants thereof. Non-limiting examples of gene products encoded by a CCL20 gene include: UniProt: P78556; NP_001123518.1 [human], NP_001005254.1 [canine], XP_003365179.2 [equine], XP 003991274.2 [feline], as well as sequence variants, isoforms encoded by alternative splicing, and various glycoforms thereof. Canonically, the protein encoded by this gene is involved in inflammatory processes and displays chemotactic activity for lymphocytes. In some instances, and merely for the sake of disambiguation, a prefix is added when referring to the protein or gene of a particular species (with h, c, e, and f, referring to the human, canine, equine, and feline forms, respectively).

The term “CXCR1 gene” refers to a mammalian gene encoding a C-X-C Motif Chemokine Receptor 1 polypeptide. Non-limiting examples of CXCR1 genes include: NCBI Gene ID: 3577 [human], NCBI Gene ID: 478906 [canine], NCBI Gene ID: 100058291 [equine], NCBI Gene ID: 101085650 [feline], as well as synonymous and non-synonymous sequence variants thereof. Non-limiting examples of gene products encoded by a CXCR1 gene include: UniProt: P25024; NP_000625.1 [human], XP_038303849.1 [canine], XP_001491062.1 [equine], XP_011283865.1 [feline], as well as sequence variants, isoforms encoded by alternative splicing, and various glycoforms thereof. Canonically, the protein encoded by this gene is a receptor for IL8 and transduces signaling to mediate neutrophil migration to sites of inflammation, among other activities. In some instances, and merely for the sake of disambiguation, a prefix is added when referring to the protein or gene of a particular species (with h, c, e, and f, referring to the human, canine, equine, and feline forms, respectively).

The term “CXCR2 gene” refers to a mammalian gene encoding a C-X-C Motif Chemokine Receptor 2 polypeptide. Non-limiting examples of CXCR2 genes include: NCBI Gene ID: 3579 [human], NCBI Gene ID: 478905 [canine], NCBI Gene ID: 100055552 [equine], NCBI Gene ID: 101085396 [feline], as well as synonymous and non-synonymous sequence variants thereof. Non-limiting examples of gene products encoded by a CXCR2 gene include: e.g., UniProt: P25025; NP_001161770.1 [human], NP_001003151.2 [canine], XP_005610662.1 [equine], XP_044890398.1 [feline], as well as sequence variants, isoforms encoded by alternative splicing, and various glycoforms thereof. Canonically, the protein encoded by this gene is a receptor for IL8 and transduces signaling to mediate neutrophil migration to sites of inflammation, among other activities. In some instances, and merely for the sake of disambiguation, a prefix is added when referring to the protein or gene of a particular species (with h, c, e, and f, referring to the human, canine, equine, and feline forms, respectively).

The term “CCR7 gene” refers to a mammalian gene encoding a C-C Motif Chemokine Receptor 7 polypeptide. Non-limiting examples of CCR7 genes include: NCBI Gene ID: 1236 [human], NCBI Gene ID: 491011 [canine], NCBI Gene ID: 100067673 [equine], NCBI Gene ID: 101084327 [feline], as well as synonymous and non-synonymous sequence variants thereof. Non-limiting examples of gene products encoded by a CCR7 gene include: UniProt: P32248; NP_001288643.1 [human], XP_038403305.1 [canine], XP_001500231.1 [equine], XP_003996882.1 [feline], as well as sequence variants, isoforms encoded by alternative splicing, and various glycoforms thereof. Canonically, the protein encoded by this gene controls the migration of memory T cells to inflamed tissues, as well as stimulate dendritic cell maturation. Signals mediated by this receptor may also function in chronic inflammation pathogenesis. In some instances, and merely for the sake of disambiguation, a prefix is added when referring to the protein or gene of a particular species (with h, c, e, and f, referring to the human, canine, equine, and feline forms, respectively).

The term “ADAM17 gene” refers to a mammalian gene encoding an ADAM Metallopeptidase Domain 17 polypeptide. Non-limiting examples of ADAM17 genes include: NCBI Gene ID: 6868 [human], NCBI Gene ID: 475662 [canine], NCBI Gene ID: 100072496 [equine], NCBI Gene ID: 101089004 [feline], as well as synonymous and non-synonymous sequence variants thereof. Non-limiting examples of gene products encoded by a ADAM17 gene include: UniProt: P78536; NP_001369706.1 [human], NP_001273795.1 [canine], NP_001295481.1 [equine], XP_003984558.1 [feline], as well as sequence variants, isoforms encoded by alternative splicing, and various glycoforms thereof. Canonically, the protein encoded by these genes is proteolytically processed to generate a mature protease, which functions by shedding the ectodomain of tumor necrosis factor-alpha, thereby releasing soluble tumor necrosis factor-alpha from its membrane-bound precursor. In some instances, and merely for the sake of disambiguation, a prefix is added when referring to the protein or gene of a particular species (with h, c, e, and f referring to the human, canine, equine, and feline forms, respectively).

The term “TNF gene” refers to a mammalian gene encoding a Tumor Necrosis Factor polypeptide. Non-limiting examples of TNF genes include: NCBI Gene ID: 7124 [human], NCBI Gene ID: 403922 [canine], NCBI Gene ID: 100033834 [equine], NCBI Gene ID: 493755 [feline], as well as synonymous and non-synonymous sequence variants thereof. Non-limiting examples of gene products encoded by a TNF gene include: UniProt: P01375; NP_000585.2 [human], NP_001003244.4 [canine], NP_001075288.2 [equine], NP_001009835.1 [feline], as well as sequence variants, isoforms encoded by alternative splicing, and various glycoforms thereof. Canonically, the protein encoded by these genes is a multifunctional proinflammatory cytokine that is mainly secreted by macrophages and can bind (and therefore function through) its receptors TNFRSF1A and TNFRSF1B. In some instances, and merely for the sake of disambiguation, a prefix is added when referring to the protein or gene of a particular species (with h, c, e, and f referring to the human, canine, equine, and feline forms, respectively).

The term “TNFRSF1A gene” refers to a mammalian gene encoding a Tumor Necrosis Factor Receptor 1 polypeptide. Non-limiting examples of TNFRSF1A genes include: NCBI Gene ID: 7132 [human], NCBI Gene ID: 403634 [canine], NCBI Gene ID: 100059548 [equine], NCBI Gene ID: 493957 [feline], as well as synonymous and non-synonymous sequence variants thereof. Non-limiting examples of gene products encoded by a TNFRSF1A gene include: UniProt: P19438; NP_001056.1 [human], XP_038295153.1 [canine], XP_023498787.1 [equine], NP_001009361.1 [feline], as well as sequence variants, isoforms encoded by alternative splicing, and various glycoforms thereof. Canonically, the proteins encoded by the genes listed above are transmembrane receptor proteins capable of binding Tumor Necrosis Factor Alpha (TNFA) or lymphotoxin alpha (LTA), its principal ligand. Upon binding to TNFA, the receptor trimerizes and is activated, transmitting intracellular signaling cascades with role in various processes, including apoptosis and inflammation. See generally, Ward-Kavanagh, L. K., et al. (2016). Immunity, 44(5), 1005-1019. In some instances, and merely for the sake of disambiguation, a prefix is added when referring to the protein or gene of a particular species (with h, c, e, and f referring to the human, canine, equine, and feline forms, respectively).

The term “TNFRSF1B gene” refers to a mammalian gene encoding a Tumor Necrosis Factor Receptor 2 polypeptide. Non-limiting examples of TNFRSF1B genes include: NCBI Gene ID: 7133 [human], NCBI Gene ID: 487437 [canine], NCBI Gene ID: 100055840 [equine], NCBI Gene ID: 101080392 [feline], as well as synonymous and non-synonymous sequence variants thereof. Non-limiting examples of gene products encoded by a TNFRSF1B gene include: UniProt: P20333; XP_011540362.1 [human], XP_038387905.1 [canine], XP_023491528.1 [equine]. XP_023113905.2 [feline], as well as sequence variants, isoforms encoded by alternative splicing, and various glycoforms thereof. Canonically, the proteins encoded by the genes listed above are transmembrane receptor proteins capable of binding TNFA or LTA and are implicated in pro-survival pathways through downstream activation of NFkB pathway. See generally, Ward-Kavanagh, L. K., et al. (2016). Immunity, 44(5), 1005-1019. In some instances, and merely for the sake of disambiguation, a prefix is added when referring to the protein or gene of a particular species (with h, c, e, and f, referring to the human, canine, equine, and feline forms, respectively).

The terms “IL4 gene” refers to a mammalian gene encoding an Interleukin 4 polypeptide. Non-limiting examples of IL4 genes include: NCBI Gene ID: 3565 [human], NCBI Gene ID: 403785 [canine], NCBI Gene ID: 100034225 [equine], NCBI Gene ID: 751514 [feline], as well as synonymous and non-synonymous sequence variants thereof. Non-limiting examples of gene products encoded by a IL4 gene include: UniProt: P05112; NP_000580.1 [human], NP_001003159.1 [canine], NP_001075988.1 [equine], NP_001036804.1 [feline], as well as sequence variants, isoforms encoded by alternative splicing, and various glycoforms thereof. Canonically, the protein encoded by these genes is a pleiotropic cytokine produced by activated T cells and is considered an important cytokine for tissue repair, counterbalancing the effects of proinflammatory type 1 cytokines, though it also promotes allergic airway inflammation and mediates acute inflammation, among other activities. In some instances, and merely for the sake of disambiguation, a prefix is added when referring to the protein or gene of a particular species (with h, c, e, and f, referring to the human, canine, equine, and feline forms, respectively).

The terms “IL4R gene” refers to a mammalian gene encoding an Interleukin 4 Receptor polypeptide. Non-limiting examples of IL4R genes include: NCBI Gene ID: 3566 [human], NCBI Gene ID: 489957 [canine], NCBI Gene ID: 791252 [equine], NCBI Gene ID: 101096277 [feline], as well as synonymous and non-synonymous sequence variants thereof. Non-limiting examples of gene products encoded by a IL4R gene include: UniProt: P24394; NP_000409.1 [human], NP_001003159.1 [canine], XP_005598791.2 [equine], XP_023102076.2 [feline], as well as sequence variants, isoforms encoded by alternative splicing, and various glycoforms thereof. Canonically, the protein encoded by these genes is a type I transmembrane protein that can bind interleukin 4 and interleukin 13 to regulate IgE production and promote differentiation of Th2 cells, among other activities. In some instances, and merely for the sake of disambiguation, a prefix is added when referring to the protein or gene of a particular species (with h, c, e, and f, referring to the human, canine, equine, and feline forms, respectively).

The terms “IL6 gene” refers to a mammalian gene encoding an Interleukin 6 polypeptide. Non-limiting examples of IL6 genes include: NCBI Gene ID: 3569 [human], NCBI Gene ID: 403985 [canine], NCBI Gene ID: 100034196 [equine], NCBI Gene ID: 493687 [feline], as well as synonymous and non-synonymous sequence variants thereof. Non-limiting examples of gene products encoded by a IL6 gene include: UniProt: P05231; NP_000591.1 [human], NP_001003301.1 [canine], NP_001075965.2 [equine], NP_001009211.2 [feline], as well as sequence variants, isoforms encoded by alternative splicing, and various glycoforms thereof. Canonically, the protein encoded by these genes is a cytokine that functions in inflammation and the maturation of B cells that is primarily produced at sites of acute and chronic inflammation, where it is secreted into the serum and induces a transcriptional inflammatory response through interleukin 6 receptor. In some instances, and merely for the sake of disambiguation, a prefix is added when referring to the protein or gene of a particular species (with h, c, e, and f, referring to the human, canine, equine, and feline forms, respectively).

The term “IL6R gene” refers to a mammalian gene encoding an Interleukin-6 Receptor polypeptide. Non-limiting examples of IL6R genes include: NCBI Gene ID: 3560 [human], NCBI Gene ID: 612271 [canine], NCBI Gene ID: 102148787 [equine], NCBI Gene ID: 101085689 [feline], as well as synonymous and non-synonymous sequence variants thereof. Non-limiting examples of gene products encoded by a IL6R gene include: UniProt: P08887; CAA41231.1 [human], XP_038527979.1 [canine], XP_023496854.1 [equine], XP_023103841.2 [feline], as well as sequence variants, isoforms encoded by alternative splicing, and various glycoforms thereof. Canonically, the proteins encoded by the genes listed above are transmembrane proteins capable of binding to interleukin-6, its native ligand. This binding event triggers intracellular signaling events that result in pro-inflammatory responses. See generally, Wolf, J., et al. (2014). Cytokine, 70(1), 11-20. In some instances, and merely for the sake of disambiguation, a prefix is added when referring to the protein or gene of a particular species (with h, c, e, and f, referring to the human, canine, equine, and feline forms, respectively).

The terms “IL6ST gene” refers to a mammalian gene encoding an Interleukin-6 Cytokine Family Signal Transducer polypeptide. Non-limiting examples of IL6ST genes include: NCBI Gene ID: 3572 [human], NCBI Gene ID: 403545 [canine], NCBI Gene ID: 100051700 [equine], NCBI Gene ID: 101089832 [feline], as well as synonymous and non-synonymous sequence variants thereof. Non-limiting examples of gene products encoded by an IL6ST gene include: UniProt: P40189; NP_001177910.1 [human], NP_001273950.1 [canine], XP_023481030.1 [equine], XP_011281205.1 [feline]), as well as sequence variants, isoforms encoded by alternative splicing, and various glycoforms thereof. Canonically, the proteins encoded by the genes listed above are signal transducers shared by many cytokines, including interleukin 6 (IL6), ciliary neurotrophic factor (CNTF), leukemia inhibitory factor (LIF), and oncostatin M (OSM) and function as a part of the cytokine receptor complex. Activation of this protein is dependent upon the binding of cytokines to their receptors (e.g., IL6 to IL6R). Knockout studies in mice suggest that this gene plays a critical role in regulating myocyte apoptosis. See generally, Martinez-Perez, C., et al. (2021). Journal of Personalized Medicine, 11(7), 618. In some instances, and merely for the sake of disambiguation, a prefix is added when referring to the protein or gene of a particular species (with h, c, e, and f, referring to the human, canine, equine, and feline forms, respectively.

The terms “IL10 gene” refers to a mammalian gene encoding an Interleukin 10 polypeptide. Non-limiting examples of IL10 genes include: NCBI Gene ID: 3586 [human], NCBI Gene ID: 403628 [canine], NCBI Gene ID: 100034187 [equine], NCBI Gene ID: 493683 [feline], as well as synonymous and non-synonymous sequence variants thereof. Non-limiting examples of gene products encoded by a IL10 gene include: UniProt: P22301; NP_000563.1 [human], NP_001003077.1 [canine], NP_001075959.1 [equine], NP_001009209.1 [feline], as well as sequence variants, isoforms encoded by alternative splicing, and various glycoforms thereof. Canonically, the protein encoded by these genes is a pleiotropic cytokine that regulates inflammation and acts on many immune cell types through binding to its heterodimeric receptor composed of IL10RA and IL10RB, thereby activating downstream signaling cascades, such as the JAK-STAT pathway. In some instances, and merely for the sake of disambiguation, a prefix is added when referring to the protein or gene of a particular species (with h, c, e, and f, referring to the human, canine, equine, and feline forms, respectively).

The term “IL10RA gene” refers to a mammalian gene encoding a Interleukin 10 Receptor Alpha polypeptide. Non-limiting examples of IL10RA genes include: NCBI Gene ID: 3587 [human], NCBI Gene ID: 610823 [canine], NCBI Gene ID: 100071172 [equine], NCBI Gene ID: 101087601 [feline], as well as synonymous and non-synonymous sequence variants thereof. Non-limiting examples of gene products encoded by a IL10RA gene include: UniProt: Q13651; NP_001549.2 [human], XP_038520677.1 [canine], XP_014596783.1 [equine], XP_003992449.1 [feline], as well as sequence variants, isoforms encoded by alternative splicing, and various glycoforms thereof. Canonically, the protein encoded by these genes is, upon forming a heterodimer with IL10RB, a regulator of pro-inflammatory signaling through the binding of its ligand IL-10. In some instances, and merely for the sake of disambiguation, a prefix is added when referring to the protein or gene of a particular species (with h, c, e, and f referring to the human, canine, equine, and feline forms, respectively).

The term “IL10RB gene” refers to a mammalian gene encoding an Interleukin 10 Receptor Beta polypeptide. Non-limiting examples of IL10RB genes include: NCBI Gene ID: 3588 [human], NCBI Gene ID: 478404 [canine], NCBI Gene ID: 100052549 [equine], NCBI Gene ID: 101090038 [feline], as well as synonymous and non-synonymous sequence variants thereof. Non-limiting examples of gene products encoded by a IL10RB gene include: UniProt: Q08334; NP_000619.3 [human], XP_038299308.1 [canine], XP_023485821.1 [equine], XP_003991512.2 [feline], as well as sequence variants, isoforms encoded by alternative splicing, and various glycoforms thereof. Canonically, the protein encoded by these genes is, upon forming a heterodimer with IL10RA, a regulator of pro-inflammatory signaling through the binding of its ligand IL-10. In some instances, and merely for the sake of disambiguation, a prefix is added when referring to the protein or gene of a particular species (with h, c, e, and f referring to the human, canine, equine, and feline forms, respectively).

The term “IL13 gene” refers to a mammalian gene encoding an Interleukin 13 polypeptide. Non-limiting examples of IL13 genes include: NCBI Gene ID: 3596 [human], NCBI Gene ID: 442990 [canine], NCBI Gene ID: 100034113 [equine], NCBI Gene ID: 101084678 [feline], as well as synonymous and non-synonymous sequence variants thereof. Non-limiting examples of gene products encoded by a IL13 gene include: UniProt: P35225; NP_001341920.1 [human], NP 001003384.1 [canine], NP_001137263.1 [equine], NP_001009209.1 [feline], as well as sequence variants, isoforms encoded by alternative splicing, and various glycoforms thereof. Canonically, the protein encoded by these genes regulates of the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL13RA1 gene” refers to a mammalian gene encoding an Interleukin 13 Receptor Alpha 1 polypeptide. Non-limiting examples of IL13RA1 genes include: NCBI Gene ID: 3597 [human], NCBI Gene ID: 403623 [canine], NCBI Gene ID: 100055312 [equine], NCBI Gene ID: 101091351 [feline], as well as synonymous and non-synonymous sequence variants thereof. Non-limiting examples of gene products encoded by a IL13RA1 gene include: UniProt: P78552; NP_001551.1 [human], XP_038306633.1 [canine], XP_023490026.1 [equine], XP_023104651.1 [feline], as well as sequence variants, isoforms encoded by alternative splicing, and various glycoforms thereof. Canonically, the protein encoded by these genes is a low affinity binding partner of IL13 and comprises a functional receptor once associated with of IL13RA2. Once bound to IL13, the receptor complex stimulates the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL13RA2 gene” refers to a mammalian gene encoding an Interleukin 13 Receptor Alpha 2 polypeptide. Non-limiting examples of IL13RA2 genes include: NCBI Gene ID: 3598 [human], NCBI Gene ID: 403622 [canine], NCBI Gene ID: 100057673 [equine], NCBI Gene ID: 101100114 [feline], as well as synonymous and non-synonymous sequence variants thereof. Non-limiting examples of gene products encoded by a IL13RA2 gene include: UniProt: Q14627; NP_000631.1 [human], NP_001003075.1 [canine], XP_023489189.1 [equine], XP_044906881.1 [feline], as well as sequence variants, isoforms encoded by alternative splicing, and various glycoforms thereof. Canonically, the protein encoded by these genes is a high affinity binding partner of IL13 but lacks a cytoplasmic domain. Along with IL13RA1, it forms a functional receptor that stimulates the production of pro-inflammatory cytokines and chemokines. In some instances, and merely for the sake of disambiguation, a prefix is added when referring to the protein or gene of a particular species (with h, c, e, and f referring to the human, canine, equine, and feline forms, respectively).

The term “IL17A gene” refers to a mammalian gene encoding an Interleukin 17A polypeptide. Non-limiting examples of IL17A genes include: NCBI Gene ID: 3605 [human], NCBI Gene ID: 481837 [canine], NCBI Gene ID: 100034142 [equine], NCBI Gene ID: 101095339 [feline], as well as synonymous and non-synonymous sequence variants thereof. Non-limiting examples of gene products encoded by a IL17A gene include: UniProt: Q16552; NP_002181.1 [human], NP_001159350.1 [canine], NP_001137264.1 [equine], XP_006931878.1 [feline], as well as sequence variants, isoforms encoded by alternative splicing, and various glycoforms thereof. Canonically, the protein encoded by these genes is an inflammatory cytokine that activates the NF kappa B signaling pathway through interactions with its heterodimeric receptor complex of IL17RA and IL17RC, thereby activating transcription of various chemokines, cytokines and other factors. In some instances, and merely for the sake of disambiguation, a prefix is added when referring to the protein or gene of a particular species (with h, c, e, and f, referring to the human, canine, equine, and feline forms, respectively).

The term “IL17RA gene” refers to a mammalian gene encoding an Interleukin 17 Receptor A polypeptide. Non-limiting examples of IL17RA genes include: NCBI Gene ID: 23765 [human], NCBI Gene ID: 486759 [canine], NCBI Gene ID: 100055511 [equine], NCBI Gene ID: 101095588 [feline], as well as synonymous and non-synonymous sequence variants thereof. Non-limiting examples of gene products encoded by a IL17RA gene include: UniProt: Q96F46; NP_001276834.1 [human], XP_038295433.1 [canine], XP_005610881.1 [equine], XP_023112364.2 [feline], as well as sequence variants, isoforms encoded by alternative splicing, and various glycoforms thereof. Canonically, the protein encoded by these genes is a transmembrane protein that binds to IL17A with low affinity as part of a multimeric receptor complex. With its ligand, IL17RA is implicated in many inflammatory conditions. In some instances, and merely for the sake of disambiguation, a prefix is added when referring to the protein or gene of a particular species (with h, c, e, and f referring to the human, canine, equine, and feline forms, respectively).

The term “IL18 gene” refers to a mammalian gene encoding an Interleukin 18 polypeptide. Non-limiting examples of IL18 genes include: NCBI Gene ID: 3606 [human], NCBI Gene ID: 403796 [canine], NCBI Gene ID: 100034216 [equine], NCBI Gene ID: 493688 [feline], as well as synonymous and non-synonymous sequence variants thereof. Non-limiting examples of gene products encoded by a IL18 gene include: UniProt: Q14116; NP_001230140.1 [human], XP 038520002.1 [canine], XP_005611483.1 [equine], NP_001009213.2 [feline], as well as sequence variants, isoforms encoded by alternative splicing, and various glycoforms thereof. Canonically, the protein encoded by these genes is a pro-inflammatory cytokine that regulates inflammatory signaling through the NF kappa B pathway when engaged with its receptor and co-receptor, IL18R1 and IL18RAP. In some instances, and merely for the sake of disambiguation, a prefix is added when referring to the protein or gene of a particular species (with h, c, e, and f, referring to the human, canine, equine, and feline forms, respectively).

The term “IL18R1 gene” refers to a mammalian gene encoding an Interleukin 18 Receptor 1 polypeptide. Non-limiting examples of IL18R1 genes include: NCBI Gene ID: 8809 [human], NCBI Gene ID: 611438 [canine], NCBI Gene ID: 100058269 [equine], NCBI Gene ID: 493938 [feline], as well as synonymous and non-synonymous sequence variants thereof. Non-limiting examples of gene products encoded by a IL18R1 gene include: UniProt: Q13478; NP_001269328.1 [human], XP_038536128.1 [canine], XP_023474273.1 [equine], NP_001009863.2 [feline], as well as sequence variants, isoforms encoded by alternative splicing, and various glycoforms thereof. Canonically, the protein encoded by these genes is an essential component for transducing IL18-mediated pro-inflammatory signaling. In some instances, and merely for the sake of disambiguation, a prefix is added when referring to the protein or gene of a particular species (with h, c, e, and f referring to the human, canine, equine, and feline forms, respectively).

The term “IL18RAP gene” refers to a mammalian gene encoding an Interleukin 18 Receptor Accessory Protein polypeptide. Non-limiting examples of IL18RAP genes include: NCBI Gene ID: 8807 [human], NCBI Gene ID: 481327 [canine], NCBI Gene ID: 100050212 [equine], NCBI Gene ID: 101084868 [feline], as well as synonymous and non-synonymous sequence variants thereof. Non-limiting examples of gene products encoded by a IL18RAP gene include: UniProt: Q53TU5; NP_001380415.1 [human], XP_038536125.1 [canine], XP_014586460.1 [equine], XP_019682529.2 [feline], as well as sequence variants, isoforms encoded by alternative splicing, and various glycoforms thereof. Canonically, the protein encoded by these genes is an accessory protein that enhances the signal transduction of IL18-mediated pro-inflammatory signaling. In some instances, and merely for the sake of disambiguation, a prefix is added when referring to the protein or gene of a particular species (with h, c, e, and f, referring to the human, canine, equine, and feline forms, respectively).

The term “NGF gene” refers to a mammalian gene encoding a Nerve Growth Factor polypeptide. Non-limiting examples of NGF genes include: NCBI Gene ID: 4803 [human], NCBI Gene ID: 403402 [canine], NCBI Gene ID: 100065669 [equine], NCBI Gene ID: 100144611 [feline], as well as synonymous and non-synonymous sequence variants thereof. Non-limiting examples of gene products encoded by a NGF gene include: UniProt: P01138; NP_002497.2 [human], XP_038546347.1 [canine], XP_001496237.2 [equine], XP_044889256.1 [feline], as well as sequence variants, isoforms encoded by alternative splicing, and various glycoforms thereof. Canonically, this secreted protein forms a functional homodimer that is incorporated into a larger complex and has nerve growth stimulating activity. The complex is also involved in the regulation of growth and the differentiation of sympathetic and certain sensory neurons. In some instances, and merely for the sake of disambiguation, a prefix is added when referring to the protein or gene of a particular species (with h, c, e, and f, referring to the human, canine, equine, and feline forms, respectively).

The term “NGFR gene” refers to a mammalian gene encoding a Nerve Growth Factor Receptor polypeptide. Non-limiting examples of NGFR genes include: NCBI Gene ID: 4804 [human], NCBI Gene ID: 491071 [canine], NCBI Gene ID: 100069694 [equine], NCBI Gene ID: 101101519 [feline], as well as synonymous and non-synonymous sequence variants thereof. Non-limiting examples of gene products encoded by a NGFR gene include: UniProt: P08138; NP_002498.1 [human], XP_038531049.1 [canine], XP_023508464.1 [equine], XP_023099534.1 [feline], as well as sequence variants, isoforms encoded by alternative splicing, and various glycoforms thereof. Canonically, the protein encoded by these genes contains four 40-amino acid repeats within its extracellular domain with 6 cysteine residues at conserved positions followed by a serine/threonine-rich region. This cysteine-rich region contains the nerve growth factor binding domain and allows for signal transduction once bound. In some instances, and merely for the sake of disambiguation, a prefix is added when referring to the protein or gene of a particular species (with h, c, e, and f, referring to the human, canine, equine, and feline forms, respectively).

The term “NTF3 gene” refers to a mammalian gene encoding a Neurotrophin-3 polypeptide. Non-limiting examples of NTF3 genes include: NCBI Gene ID: 4908 [human], NCBI Gene ID: 493963 [canine], NCBI Gene ID: 100051839 [equine], NCBI Gene ID: 486731 [feline], as well as synonymous and non-synonymous sequence variants thereof. Non-limiting examples of gene products encoded by a NTF3 gene include: UniProt: P20783; NP_001096124.1 [human], XP 038293846.1 [canine], XP_023498780.1 [equine], NP_001009367.1 [feline], as well as sequence variants, isoforms encoded by alternative splicing, and various glycoforms thereof. Canonically, the protein encoded by these genes controls survival and differentiation of neurons. In some instances, and merely for the sake of disambiguation, a prefix is added when referring to the protein or gene of a particular species (with h, c, e, and f referring to the human, canine, equine, and feline forms, respectively).

The term “NTF4 gene” refers to a mammalian gene encoding a Neurotrophin-4 polypeptide. Non-limiting examples of NTF4 genes include: NCBI Gene ID: 4909 [human], NCBI Gene ID: 611987 [canine], NCBI Gene ID: 100054859 [equine], NCBI Gene ID: 101100428 [feline], as well as synonymous and non-synonymous sequence variants thereof. Non-limiting examples of gene products encoded by a NTF4 gene include: UniProt: P34130; NP_001382418.1 [human], NP 001177358.2 [canine], XP_023505846.1 [equine], XP_023101354.2 [feline], as well as sequence variants, isoforms encoded by alternative splicing, and various glycoforms thereof. Canonically, the protein encoded by these genes is proteolytically processed to a mature form, which can promote survival of neurons through binding of its cognate receptor. Dysregulation of this protein is observed in various neurological disorders. In some instances, and merely for the sake of disambiguation, a prefix is added when referring to the protein or gene of a particular species (with h, c, e, and f referring to the human, canine, equine, and feline forms, respectively).

The term “NTRK1 gene” refers to a mammalian gene encoding a Neurotrophic Receptor Tyrosine Kinase 1 polypeptide. Non-limiting examples of NTRK1 genes include: NCBI Gene ID: 4914 [human], NCBI Gene ID: 490404 [canine], NCBI Gene ID: 100064594 [equine], NCBI Gene ID: 101081603 [feline], as well as synonymous and non-synonymous sequence variants thereof. Non-limiting examples of gene products encoded by a NTRK1 gene include: UniProt: P04629; NP_001007793.1 [human], XP 038527745.1 [canine], XP_023496742.1 [equine], XP_023103311.1 [feline], as well as sequence variants, isoforms encoded by alternative splicing, and various glycoforms thereof. Canonically, the protein encoded by these genes is a membrane-bound receptor that binds neutrophin and signals through the MAPK pathway to regulate cell differentiation, among other functions. In some instances, and merely for the sake of disambiguation, a prefix is added when referring to the protein or gene of a particular species (with h, c, e, and f referring to the human, canine, equine, and feline forms, respectively).

The term “NTRK2 gene” refers to a mammalian gene encoding a Neurotrophic Receptor Tyrosine Kinase 2 polypeptide. Non-limiting examples of NTRK2 genes include: NCBI Gene ID: 4915 [human], NCBI Gene ID: 484147 [canine], NCBI Gene ID: 100061700 [equine], NCBI Gene ID: 101101347 [feline], as well as synonymous and non-synonymous sequence variants thereof. Non-limiting examples of gene products encoded by a NTRK2 gene include: UniProt: Q16620; NP_001007098.1 [human], XP 038510982.1 [canine], XP_023482906.1 [equine], XP_023097987.1 [feline], as well as sequence variants, isoforms encoded by alternative splicing, and various glycoforms thereof. Canonically, the protein encoded by these genes is a membrane-bound receptor that binds neutrophin and signals through the MAPK pathway to regulate cell differentiation, among other functions. In some instances, and merely for the sake of disambiguation, a prefix is added when referring to the protein or gene of a particular species (with h, c, e, and f referring to the human, canine, equine, and feline forms, respectively).

The term “BDNF gene” refers to a mammalian gene encoding a Brain-Derived Neurotrophic Factor polypeptide. Non-limiting examples of BDNF genes include: NCBI Gene ID: 627 [human], NCBI Gene ID: 403461 [canine], NCBI Gene ID: 100009689 [equine], NCBI Gene ID: 493690 [feline], as well as synonymous and non-synonymous sequence variants thereof. Non-limiting examples of gene products encoded by a BDNF gene include: UniProt: P23560; NP_001137277.1 [human], NP_001002975.1 [canine], NP_001075256.1 [equine], NP_001009828.1 [feline], as well as sequence variants, isoforms encoded by alternative splicing, and various glycoforms thereof. Canonically, the protein encoded by these genes is proteolytically processed to a mature form, which can promote survival of neurons through binding of its cognate receptor. Dysregulation of this protein is observed in various neurological disorders. In some instances, and merely for the sake of disambiguation, a prefix is added when referring to the protein or gene of a particular species (with h, c, e, and f referring to the human, canine, equine, and feline forms, respectively).

The terms “SCN1A gene” refers to a mammalian gene encoding a Sodium Voltage-Gated Channel Alpha 1 polypeptide. Non-limiting examples of SCN1A genes include: NCBI Gene ID: 6323 [human], NCBI Gene ID: 478775 [canine], NCBI Gene ID: 100052059 [equine], NCBI Gene ID: 101081823 [feline], as well as synonymous and non-synonymous sequence variants thereof. Non-limiting examples of gene products encoded by a SCN1A gene include: UniProt: P35498; NP_001159435.1 [human], XP_038302870.1 [canine], XP_023478839.1 [equine], XP_019693764.1 [feline], as well as sequence variants, isoforms encoded by alternative splicing, and various glycoforms thereof. Canonically, the protein encoded by these genes mediates the voltage-dependent sodium ion permeability of excitable membranes and is involved in sensory perception of mechanical pain (i.e., activation in somatosensory neurons has been shown to induce pain without neurogenic inflammation). In some instances, and merely for the sake of disambiguation, a prefix is added when referring to the protein or gene of a particular species (with h, c, e, and f referring to the human, canine, equine, and feline forms, respectively).

The terms “SCN2A gene” refers to a mammalian gene encoding a Sodium Voltage-Gated Channel Alpha 2 polypeptide. Non-limiting examples of SCN2A genes include: NCBI Gene ID: 6326 [human], NCBI Gene ID: 478773 [canine], NCBI Gene ID: 100051816 [equine], NCBI Gene ID: 101080472 [feline], as well as synonymous and non-synonymous sequence variants thereof. Non-limiting examples of gene products encoded by a SCN2A gene include: UniProt: Q99250; NP_001035232.1 [human], XP 038302857.1 [canine], XP_023478830.1 [equine], XP_023115179.1 [feline], as well as sequence variants, isoforms encoded by alternative splicing, and various glycoforms thereof. Canonically, the protein encoded by these genes mediates the voltage-dependent sodium ion permeability of excitable membranes. In some instances, and merely for the sake of disambiguation, a prefix is added when referring to the protein or gene of a particular species (with h, c, e, and f, referring to the human, canine, equine, and feline forms, respectively).

The term “SCN3A gene” refers to a mammalian gene encoding a Sodium Voltage-Gated Channel Alpha 3 polypeptide. Non-limiting examples of SCN3A genes include: NCBI Gene ID: 6328 [human], NCBI Gene ID: 478772 [canine], NCBI Gene ID: 100061941 [equine], NCBI Gene ID: 101082587 [feline], as well as synonymous and non-synonymous sequence variants thereof. Non-limiting examples of gene products encoded by a SCN3A gene include: UniProt: Q9NY46; NP_001075145.1 [human], XP_038302852.1 [canine], XP_023478823.1 [equine], XP_019693750.1 [feline], as well as sequence variants, isoforms encoded by alternative splicing, and various glycoforms thereof. Canonically, the protein encoded by these genes is a subunit of voltage-gated sodium channels and is responsible for propagation of action potentials in neurons and muscle tissue. In some instances, and merely for the sake of disambiguation, a prefix is added when referring to the protein or gene of a particular species (with h, c, e, and f, referring to the human, canine, equine, and feline forms, respectively).

The terms “SCN4A gene” refers to a mammalian gene encoding a Sodium Voltage-Gated Channel Alpha 4 polypeptide. Non-limiting examples of SCN4A genes include: NCBI Gene ID: 6328 [human], NCBI Gene ID: 119873250 [canine], NCBI Gene ID: 100049793 [equine], NCBI Gene ID: 101098669 [feline], as well as synonymous and non-synonymous sequence variants thereof. Non-limiting examples of gene products encoded by a SCN4A gene include: UniProt: Q9NY46; NP_001075145.1 [human], XP_038531923.1 [canine], NP_001075230.2 [equine], XP_006940553.1 [feline], as well as sequence variants, isoforms encoded by alternative splicing, and various glycoforms thereof. Canonically, the protein encoded by these genes is a subunit of voltage-gated sodium channels and is responsible for propagation of action potentials in neurons and muscle tissue. In some instances, and merely for the sake of disambiguation, a prefix is added when referring to the protein or gene of a particular species (with h, c, e, and f, referring to the human, canine, equine, and feline forms, respectively).

The terms “SCN5A gene” refers to a mammalian gene encoding a Sodium Voltage-Gated Channel Alpha 5 polypeptide. Non-limiting examples of SCN5A genes include: NCBI Gene ID: 6331 [human], NCBI Gene ID: 403497 [canine], NCBI Gene ID: 100034027 [equine], NCBI Gene ID: 101100994 [feline], as well as synonymous and non-synonymous sequence variants thereof. Non-limiting examples of gene products encoded by a SCN5A gene include: UniProt: Q14524; NP_000326.2 [human], NP_001002994.1 [canine], NP_001157367.1 [equine], XP_044893792.1 [feline], as well as sequence variants, isoforms encoded by alternative splicing, and various glycoforms thereof. Canonically, the protein encoded by these genes is a subunit of voltage-gated sodium channels and is found primarily in cardiac muscle and is responsible for the initial upstroke of the action potential in an electrocardiogram. In some instances, and merely for the sake of disambiguation, a prefix is added when referring to the protein or gene of a particular species (with h, c, e, and f, referring to the human, canine, equine, and feline forms, respectively).

The term “SCN8A gene” refers to a mammalian gene encoding a Sodium Voltage-Gated Channel Alpha 8 polypeptide. Non-limiting examples of SCN8A genes include: NCBI Gene ID: 6335 [human], NCBI Gene ID: 477604 [canine], NCBI Gene ID: 100052777 [equine], NCBI Gene ID: 101096578 [feline], as well as synonymous and non-synonymous sequence variants thereof. Non-limiting examples of gene products encoded by a SCN8A gene include: UniProt: Q9UQD0; NP_001171455.1 [human], XP_038294063.1 [canine], XP_023499351.1 [equine], XP_023112849.1 [feline], as well as sequence variants, isoforms encoded by alternative splicing, and various glycoforms thereof. Canonically, the protein encoded by these genes is the ion pore subunit of the voltage-gated sodium channel and is essential for rapid membrane depolarization during neuronal action potentials. In some instances, and merely for the sake of disambiguation, a prefix is added when referring to the protein or gene of a particular species (with h, c, e, and f, referring to the human, canine, equine, and feline forms, respectively).

The term “SCN9A gene” refers to a mammalian gene encoding a Sodium Voltage-Gated Channel Alpha 9 polypeptide. Non-limiting examples of SCN9A genes include: NCBI Gene ID: 6335 [human], NCBI Gene ID: 100855710 [canine], NCBI Gene ID: 100052120 [equine], NCBI Gene ID: 101082841 [feline], as well as synonymous and non-synonymous sequence variants thereof. Non-limiting examples of gene products encoded by a SCN9A gene include: UniProt: Q15858; NP_001352465.1 [human], XP_038302872.1 [canine], XP_023478844.1 [equine], XP_044889827.1 [feline], as well as sequence variants, isoforms encoded by alternative splicing, and various glycoforms thereof. Canonically, the protein encoded by these genes is a voltage-dependent sodium ion channel that has been associated with various pain disorders, especially in the development of inflammatory pain. In some instances, and merely for the sake of disambiguation, a prefix is added when referring to the protein or gene of a particular species (with h, c, e, and f, referring to the human, canine, equine, and feline forms, respectively).

The term “SCN10A gene” refers to a mammalian gene encoding a Sodium Voltage-Gated Channel Alpha 10 polypeptide. Non-limiting examples of SCN10A genes include: NCBI Gene ID: 6336 [human], NCBI Gene ID: 477026 [canine], NCBI Gene ID: 100055493 [equine], NCBI Gene ID: 101085569 [feline], as well as synonymous and non-synonymous sequence variants thereof. Non-limiting examples of gene products encoded by a SCN10A gene include: UniProt: Q9Y5Y9; NP_001280235.2 [human], NP_001003203.1 [canine], XP_014587037.1 [equine], XP_044893784.1 [feline], as well as sequence variants, isoforms encoded by alternative splicing, and various glycoforms thereof. Canonically, the protein encoded by these genes is a membrane-spanning subunit of voltage-dependent sodium channels that may be involved in the onset of pain associated with neuropathies. In some instances, and merely for the sake of disambiguation, a prefix is added when referring to the protein or gene of a particular species (with h, c, e, and f, referring to the human, canine, equine, and feline forms, respectively).

The term “SCN11A gene” refers to a mammalian gene encoding a Sodium Voltage-Gated Channel Alpha 11 polypeptide. Non-limiting examples of SCN11A genes include: NCBI Gene ID: 11280 [human], NCBI Gene ID: 485593 [canine], NCBI Gene ID: 100068480 [equine], NCBI Gene ID: 101085312 [feline], as well as synonymous and non-synonymous sequence variants thereof. Non-limiting examples of gene products encoded by a SCN11A gene include: UniProt: Q9UI33; NP_001336182.1 [human], XP_038426400.1 [canine], XP_001916634.3 [equine], XP_044893782.1 [feline], as well as sequence variants, isoforms encoded by alternative splicing, and various glycoforms thereof. Canonically, the protein encoded by these genes is a membrane-spanning subunit of voltage-dependent sodium channels and is highly expressed in nociceptive neurons of dorsal root ganglia and trigeminal ganglia. Mutations in the SCN11A gene have been associated with various pain disorders. In some instances, and merely for the sake of disambiguation, a prefix is added when referring to the protein or gene of a particular species (with h, c, e, and f referring to the human, canine, equine, and feline forms, respectively).

The terms “TAC1 gene” refers to a mammalian gene encoding a Tachykinin Precursor 1 polypeptide. Non-limiting examples of TAC1 genes include: NCBI Gene ID: 6863 [human], NCBI Gene ID: 475239 [canine], NCBI Gene ID: 100052324 [equine], NCBI Gene ID: 101095481 [feline], as well as synonymous and non-synonymous sequence variants thereof. Non-limiting examples of gene products encoded by a TAC1 gene include: UniProt: P20366; NP_003173.1 [human], XP_038541905.1 [canine], XP_014594521.1 [equine], XP_003982840.1 [feline], as well as sequence variants, isoforms encoded by alternative splicing, and various glycoforms thereof. Canonically, the protein encoded by these genes is a precursor for four products of the tachykinin peptide hormone family-substance P, neurokinin A, neuropeptide K and neuropeptide gamma. These hormones are thought to function as neurotransmitters that interact with nerve receptors and smooth muscle cells. In some instances, and merely for the sake of disambiguation, a prefix is added when referring to the protein or gene of a particular species (with h, c, e, and f, referring to the human, canine, equine, and feline forms, respectively).

The terms “TAC3 gene” refers to a mammalian gene encoding a Tachykinin Precursor 3 polypeptide. Non-limiting examples of TAC3 genes include: NCBI Gene ID: 6866 [human], NCBI Gene ID: 607315 [canine], NCBI Gene ID: 100052722 [equine], NCBI Gene ID: 101089368 [feline], as well as synonymous and non-synonymous sequence variants thereof. Non-limiting examples of gene products encoded by a TAC3 gene include: UniProt: Q9UHF0; NP_001171525.1 [human], UniProt: A0A8I3N7Z8; NP_001362511.2 canine], XP_023499603.1 [equine], XP_019690663.1 [feline], as well as sequence variants, isoforms encoded by alternative splicing, and various glycoforms thereof. Canonically, the protein encoded by these genes is proteolytically processed to generate a mature peptide, which is primarily expressed in the central and peripheral nervous systems and functions as a neurotransmitter. This peptide is the ligand for the neurokinin-3 receptor. These hormones are thought to function as neurotransmitters that interact with nerve receptors and smooth muscle cells. In some instances, and merely for the sake of disambiguation, a prefix is added when referring to the protein or gene of a particular species (with h, c, e, and f referring to the human, canine, equine, and feline forms, respectively).

The term “TACR1 gene” refers to a mammalian gene encoding a Tachykinin Receptor 1 polypeptide. Non-limiting examples of TACR1 genes include: NCBI Gene ID: 6869 [human], NCBI Gene ID: 403815 [canine], NCBI Gene ID: 100053491 [equine], NCBI Gene ID: 101090094 [feline], as well as synonymous and non-synonymous sequence variants thereof. Non-limiting examples of gene products encoded by a TACR1 gene include: UniProt: P25103; NP_001049.1 [human], NP_001012637.1 canine], XP_001499730.1 [equine], XP_003984209.1 [feline], as well as sequence variants, isoforms encoded by alternative splicing, and various glycoforms thereof. Canonically, the protein encoded by these genes is the receptor for the tachykinin substance P, also referred to as neurokinin 1. TACR1 activates a phosphatidylinositol-calcium second messenger system and can also bind substance K and neuromedin-K with less affinity. In some instances, and merely for the sake of disambiguation, a prefix is added when referring to the protein or gene of a particular species (with h, c, e, and f referring to the human, canine, equine, and feline forms, respectively).

The term “TACR2 gene” refers to a mammalian gene encoding a Tachykinin Receptor 2 polypeptide. Non-limiting examples of TACR2 genes include: NCBI Gene ID: 6865 [human], NCBI Gene ID: 489020 [canine], NCBI Gene ID: 100034168 [equine], NCBI Gene ID: 101094541 [feline]], as well as synonymous and non-synonymous sequence variants thereof. Non-limiting examples of gene products encoded by a TACR2 gene include: UniProt: P21452; NP_001048.2 [human], NP_001012635.1 [canine], XP_001502752.2 [equine], XP_044896003.1 [feline], as well as sequence variants, isoforms encoded by alternative splicing, and various glycoforms thereof. Canonically, the protein encoded by these genes is the receptor for the tachykinin substance K, also referred to as neurokinin A. TACR2 activates a phosphatidylinositol-calcium second messenger system and can also bind neuromedin-K and substance P with less affinity. In some instances, and merely for the sake of disambiguation, a prefix is added when referring to the protein or gene of a particular species (with h, c, e, and f, referring to the human, canine, equine, and feline forms, respectively).

The term “TACR3 gene” refers to a mammalian gene encoding a Tachykinin Receptor 3 polypeptide. Non-limiting examples of TACR3 genes include: NCBI Gene ID: 6870 [human], NCBI Gene ID: 403814 [canine], NCBI Gene ID: 100073088 [equine], NCBI Gene ID: 101093603 [feline], as well as synonymous and non-synonymous sequence variants thereof. Non-limiting examples of gene products encoded by a TACR3 gene include: UniProt: P29371; NP_001050.1 [human], NP_001091010.1 [canine], XP_023492571.1 [equine], XP_003985169.3 [feline], as well as sequence variants, isoforms encoded by alternative splicing, and various glycoforms thereof. Canonically, the protein encoded by these genes is the receptor for the tachykinin neurokinin 3, also referred to as neurokinin B or neuromedin-K. TACR3 activates a phosphatidylinositol-calcium second messenger system and can also bind substance K and substance P with less affinity. In some instances, and merely for the sake of disambiguation, a prefix is added when referring to the protein or gene of a particular species (with h, c, e, and f referring to the human, canine, equine, and feline forms, respectively).

The term “MRGPRX2 gene” refers to a mammalian gene encoding a MAS related GPR family member X2 polypeptide. Non-limiting examples of MRGPRX2 genes include: NCBI Gene ID: 117194 [human], NCBI Gene ID: 485410 [canine], NCBI Gene ID: 100071950 [equine], NCBI Gene ID: 101097092 [feline]) or an encoded gene product (e.g., UniProt: Q96LB1; NP_001290544.1 [human], XP_038285538.1 [canine], XP_023501936.1 [equine], XP_003993155.1 [feline], as well as sequence variants, isoforms encoded by alternative splicing, and various glycoforms thereof. Canonically, the protein encoded by these genes enables G protein-coupled receptor activity and neuropeptide binding activity and is involved in mast cell degranulation and positive regulation of cytokinesis. In some instances, and merely for the sake of disambiguation, a prefix is added when referring to the protein or gene of a particular species (with h, c, e, and f referring to the human, canine, equine, and feline forms, respectively).

The term “ATP1A1 gene” refers to a mammalian gene encoding a ATPase Na+/K+ transporting subunit alpha 1 polypeptide. Non-limiting examples of ATP1A1 genes include: NCBI Gene ID: 476 [human], NCBI Gene ID: 403992 [canine], NCBI Gene ID: 100034139 [equine], NCBI Gene ID: 101083695 [feline], as well as synonymous and non-synonymous sequence variants thereof. Non-limiting examples of gene products encoded by a ATP1A1 gene include: UniProt: P05023; NP_000692.2 [human], NP_001376153.1 [canine], NP_001108004.2 [equine], XP_011283388.1 [feline], as well as sequence variants, isoforms encoded by alternative splicing, and various glycoforms thereof. Canonically, the protein encoded by these genes is an integral membrane protein subunit of the complex responsible for establishing and maintaining the electrochemical gradients of Na and K ions across a plasma membrane, which is essential for osmoregulation and electrical excitability of nerve and muscle. In some instances, and merely for the sake of disambiguation, a prefix is added when referring to the protein or gene of a particular species (with h, c, e, and f, referring to the human, canine, equine, and feline forms, respectively).

The term “CALCA gene” refers to a mammalian gene encoding a Calcitonin Related Polypeptide Alpha polypeptide. Non-limiting examples of CALCA genes include: NCBI Gene ID: 796 [human], NCBI Gene ID: 403946 [canine], NCBI Gene ID: 100033906 [equine], NCBI Gene ID: 101095582 [feline], as well as synonymous and non-synonymous sequence variants thereof. Non-limiting examples of gene products encoded by a CALCA gene include: UniProt: P01258; NP_001029124.1 [human], NP_001300719.1 [canine], NP_001075323.1 [equine], XP_019667660.1 [feline], as well as sequence variants, isoforms encoded by alternative splicing, and various glycoforms thereof. Canonically, this gene encodes multiple gene products, such as calcitonin, calcitonin gene-related peptide and katacalcin, through tissue-specific alternative RNA splicing of the gene transcripts and cleavage of inactive precursor proteins. The proteins are involved in calcium regulation, regulate phosphorus metabolism, and function as a vasodilator, among other functions. In some instances, and merely for the sake of disambiguation, a prefix is added when referring to the protein or gene of a particular species (with h, c, e, and f referring to the human, canine, equine, and feline forms, respectively).

The term “CALCB gene” refers to a mammalian gene encoding a Calcitonin Related Polypeptide Beta polypeptide. Non-limiting examples of CALCB genes include: NCBI Gene ID: 797 [human], NCBI Gene ID: 403415 [canine], NCBI Gene ID: 100034126 [equine], NCBI Gene ID: 101094539 [feline], as well as synonymous and non-synonymous sequence variants thereof. Non-limiting examples of gene products encoded by a CALCB gene include: UniProt: P10092; NP_000719.1 [human], NP_001002948.1 [canine], NP_001075397.1 [equine], XP_044894937.1 [feline], as well as sequence variants, isoforms encoded by alternative splicing, and various glycoforms thereof. Canonically, the protein encoded by these genes acts as a vasodilator and a neurotransmitter, among other functions. In some instances, and merely for the sake of disambiguation, a prefix is added when referring to the protein or gene of a particular species (with h, c, e, and f, referring to the human, canine, equine, and feline forms, respectively).

The terms “CALCRL gene” refers to a mammalian gene encoding a Calcitonin Receptor Like Receptor polypeptide. Non-limiting examples of CALCRL genes include: NCBI Gene ID: 10203 [human], NCBI Gene ID: 488438 [canine], NCBI Gene ID: 100054281 [equine], NCBI Gene ID: 101086333 [feline], as well as synonymous and non-synonymous sequence variants thereof. Non-limiting examples of gene products encoded by a CALCRL gene include: UniProt: Q16602; NP_001258680.1 [human], XP_038303202.1 [canine], XP_023477941.1 [equine], XP_011283721.1 [feline], as well as sequence variants, isoforms encoded by alternative splicing, and various glycoforms thereof. Canonically, the protein encoded by these genes comprises the receptor for CGRP (with RAMP1) and receptor for ADM (with RAMP2/3) and activates adenylyl cyclase. In some instances, and merely for the sake of disambiguation, a prefix is added when referring to the protein or gene of a particular species (with h, c, e, and f, referring to the human, canine, equine, and feline forms, respectively).

The term “RAMP1 gene” refers to a mammalian gene encoding a Receptor Activity Modifying Protein 1 polypeptide. Non-limiting examples of RAMP1 genes include: NCBI Gene ID: 10267 [human], NCBI Gene ID: 607163 [canine], NCBI Gene ID: 100066550 [equine], NCBI Gene ID: 101092133 [feline], as well as synonymous and non-synonymous sequence variants thereof. Non-limiting examples of gene products encoded by a RAMP1 gene include: UniProt: 060894; NP_005846.1 [human], XP_038291846.1 [canine], XP_023498460.1 [equine], XP_044890618.1 [feline], as well as sequence variants, isoforms encoded by alternative splicing, and various glycoforms thereof. Canonically, the protein encoded by these genes is required to transport calcitonin-receptor-like receptor (CRLR) to the plasma membrane and, with CRLR, functions as a CGRP receptor. In some instances, and merely for the sake of disambiguation, a prefix is added when referring to the protein or gene of a particular species (with h, c, e, and f, referring to the human, canine, equine, and feline forms, respectively).

The term “ADM gene” refers to a mammalian gene encoding an Adrenomedullin polypeptide. Non-limiting examples of ADM genes include: NCBI Gene ID: 133 [human], NCBI Gene ID: 403817 [canine], NCBI Gene ID: 100033857 [equine], NCBI Gene ID: 101087095 [feline], as well as synonymous and non-synonymous sequence variants thereof. Non-limiting examples of gene products encoded by a ADM gene include: UniProt: P35318; NP_001115.1 [human], NP_001003183.1 [canine], NP_001157351.1 [equine], XP_044894880.1 [feline], as well as sequence variants, isoforms encoded by alternative splicing, and various glycoforms thereof. Canonically, the protein encoded by this gene is a 52 aa peptide with several functions, including vasodilation, regulation of hormone secretion, promotion of angiogenesis. In some instances, and merely for the sake of disambiguation, a prefix is added when referring to the protein or gene of a particular species (with h, c, e, and f referring to the human, canine, equine, and feline forms, respectively).

The term “CRCP gene” refers to a mammalian gene encoding a CGRP Receptor Component polypeptide. Non-limiting examples of CRCP genes include: NCBI Gene ID: 27297 [human], NCBI Gene ID: 479705 [canine], NCBI Gene ID: 100061681 [equine], NCBI Gene ID: 101084503 [feline], as well as synonymous and non-synonymous sequence variants thereof. Non-limiting examples of gene products encoded by a CRCP gene include: UniProt: 075575; NP_001035737.1 [human], XP_038523718.1 [canine], XP_001493592.3 [equine], XP_044903465.1 [feline], as well as sequence variants, isoforms encoded by alternative splicing, and various glycoforms thereof. Canonically, the protein encoded by this gene is an accessory protein for the CGRP receptor that modulates CGRP responsiveness in a variety of tissues. In some instances, and merely for the sake of disambiguation, a prefix is added when referring to the protein or gene of a particular species (with h, c, e, and f referring to the human, canine, equine, and feline forms, respectively).

The term “YAP1 gene” refers to a mammalian gene encoding a Yes1-Associated Protein polypeptide. Non-limiting examples of YAP1 genes include: NCBI Gene ID: 10413 [human], NCBI Gene ID: 479465 [canine], NCBI Gene ID: 100068834 [equine], NCBI Gene ID: 101101408 [feline], as well as synonymous and non-synonymous sequence variants thereof. Non-limiting examples of gene products encoded by a YAP1 gene include: UniProt: P46937; NP_001123617.1 [human], XP 038521022.1 [canine], XP_023500466.1 [equine], XP_044894121.1 [feline], as well as sequence variants, isoforms encoded by alternative splicing, and various glycoforms thereof. Canonically, the protein encoded by this gene is involved in development, growth, repair and homeostasis. In some instances, and merely for the sake of disambiguation, a prefix is added when referring to the protein or gene of a particular species (with h, c, e, and f, referring to the human, canine, equine, and feline forms, respectively).

The term “IL1RAP gene” refers to a mammalian gene encoding an Interleukin 1 Receptor Accessory Protein polypeptide. Non-limiting examples of ILRAP1 genes include: NCBI Gene ID: 3556 [human], NCBI Gene ID: 488126 [canine], NCBI Gene ID: 100068726 [equine], NCBI Gene ID: 101094125 [feline], as well as synonymous and non-synonymous sequence variants thereof. Non-limiting examples of gene products encoded by a IL1RAP gene include: UniProt: Q9NPH3; NP_002173.1 [human], XP_038318680.1 [canine], XP_001498597.2 [equine], XP 044893081.1 [feline], as well as sequence variants, isoforms encoded by alternative splicing, and various glycoforms thereof. Canonically, the proteins encoded by the genes listed above are capable of associating with IL1R1 bound to IL1 to form the high affinity interleukin-1 receptor complex that mediates interleukin-1-dependent activation of NF-kappa-B and other signaling pathways through the recruitment of adapter molecules such as TOLL1P, MYD88, and IRAK1 or IRAK2 via TIR-TIR interactions with the cytoplasmic domains of receptor/coreceptor subunits. In some instances, and merely for the sake of disambiguation, a prefix is added when referring to the protein or gene of a particular species (with h, c, e, and f, referring to the human, canine, equine, and feline forms, respectively).

The term “IL1R1 gene” refers to a mammalian gene encoding an Interleukin 1 receptor type 1 polypeptide. Non-limiting examples of IL1R1 genes include: NCBI Gene ID: 3554 [human], NCBI Gene ID: 481328 [canine], NCBI Gene ID: 100009699 [equine], NCBI Gene ID: 101080705 [feline], as well as synonymous and non-synonymous sequence variants thereof. Non-limiting examples of gene products encoded by a ILR1 gene include: UniProt: P14778; NP_001307909.1 [human], XP 038536135.1 [canine], NP_001075263.2 [equine], XP_023107327.2 [feline], as well as sequence variants, isoforms encoded by alternative splicing, and various glycoforms thereof. Canonically, the proteins encoded by the genes listed above are capable of binding all forms of the pro-inflammatory cytokine interleukin 1 (IL1 or IL1) to mediate interleukin-1-dependent activation of NF-kappa-B, MAPK and other signaling pathways. This intracellular signaling involves the recruitment of adapter molecules such as TOLLIP, MYD88, and IRAK1 or IRAK2 via TIR-TIR interactions with the cytoplasmic domains of receptor/coreceptor subunits. IL1R1 can also bind the Interleukin 1 receptor antagonist (IL1Ra or IL1Ra or IL1RN), which prevents association with IL1RAP to form a signaling-competent complex. In some instances, and merely for the sake of disambiguation, a prefix is added when referring to the protein or gene of a particular species (with h, c, e, and f referring to the human, canine, equine, and feline forms, respectively).

The term “ILIA gene” refers to a mammalian gene encoding a Interleukin 1 Alpha polypeptide. Non-limiting examples of IL1A genes include: NCBI Gene ID: 3552 [human], NCBI Gene ID: 403782 [canine], NCBI Gene ID: 100064969 [equine], NCBI Gene ID: 493944 [feline], as well as synonymous and non-synonymous sequence variants thereof. Non-limiting examples of gene products encoded by a IL1A gene include: UniProt: P01583; NP_000566.3 [human], NP_001003157.2 [canine], NP_001075969.2 [equine], NP_001009351.1 [feline], as well as sequence variants, isoforms encoded by alternative splicing, and various glycoforms thereof. Canonically, the proteins encoded by the genes listed above are pro-inflammatory cytokines that signal through interaction with IL1R1 and IL1RAP to activate various pathways, including MAPK, JNK and NF-kappa B. In some instances, and merely for the sake of disambiguation, a prefix is added when referring to the protein or gene of a particular species (with h, c, e, and f referring to the human, canine, equine, and feline forms, respectively).

The terms “IL1B gene” refers to a mammalian gene encoding an Interleukin 1 Beta polypeptide. Non-limiting examples of IL1B genes include: NCBI Gene ID: 3553 [human], NCBI Gene ID: 403974 [canine], NCBI Gene ID: 100034237 [equine], NCBI Gene ID: 768274 [feline], as well as synonymous and non-synonymous sequence variants thereof. Non-limiting examples of gene products encoded by an IL1B gene include: UniProt: P01584; NP_000567.1 [human], NP_001033060.1 [canine], NP_001075995.1 [equine], NP_001070882.1 [feline], as well as sequence variants, isoforms encoded by alternative splicing, and various glycoforms thereof. Canonically, the protein encoded by the genes listed above is a major mediator of the inflammatory response and pyrogen that signals through interaction with IL1R1 and IL1RAP. In the central nervous system (CNS) IL1B has been shown to contribute to inflammatory pain hypersensitivity, among other pathologies. In some instances, and merely for the sake of disambiguation, a prefix is added when referring to the protein or gene of a particular species (with h, c, e, and f referring to the human, canine, equine, and feline forms, respectively).

The term “treatment” refers to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. For example, a composition, method, or system of the present disclosure may be administered as a prophylactic treatment to a subject that has a predisposition for a given condition (e.g., arthritis). “Treatment”, as used herein, covers any treatment of a disease in a mammal, particularly in a human, canine, feline, or equine, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms.

“Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine. It is understood that compositions and methods of the present disclosure are applicable to treat all mammals, including, but not limited to human, canine, feline, equine, and bovine subjects.

The term “therapeutically effective” refers to the amount of a composition or combination of compositions as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular composition(s) chosen, the dosing regimen to be followed, whether the composition is administered in combination with other compositions or compounds, timing of administration, the tissue to which it is administered, and the physical delivery system in which the composition is carried.

A “spinal condition or disorder” includes, but is not limited to, low back pain, neck pain, discogenic disorders, adolescent idiopathic scoliosis, adult degenerative scoliosis, cervical degenerative disc disease, cervical disc herniation, cervical myelopathy, cervical stenosis, compression fractures, degenerative spondylolisthesis, isthmic spondylolisthesis, low back sprains and strains, lumbar degenerative disc disease, lumbar disc hemiation, lumbar stenosis, neck sprain (whiplash) and strain, neck strain, osteoporosis, and whiplash. Generally, such disorders or conditions contribute to or cause localized nociception, inflammation, or morphological changes (e.g., fibrosis, degeneration, osteolysis, osteogenesis) at the cervical, thoracic, lumbar or sacral spine, or surrounding tissues.

“Low back pain” is defined as measurable or discernible pain or discomfort (either chronic or sporadic) in a given subject, encompassing at least the lumbar-spinal region of a mammal. The pain may present as being localized to the lower back (e.g., muscle ache) or as shooting, burning, stinging, and/or radiating sensations throughout the subject's back and/or extremities. The pain may be idiopathic or may be associated with one or more (diagnosed or undiagnosed) underlying conditions including, but not limited to degenerative disc disease, chronic inflammation, arthritis, osteoporosis, trauma (e.g., post-surgical), infection (e.g., discospondylitis), neuropathies, musculo-skeletal abnormalities (e.g., slipped discs or spinal stenosis or spondylolisthesis), herniated nucleus pulposus (HNP), annular ligament tears, facet joint arthritis, radicular nerve compression, and/or other degenerative disorders.

“Neck pain” is defined as measurable or discernable pain or discomfort associated with the cervical spine or adjacent ligaments, muscles, and/or tendons. The pain may manifest as localized pain in the neck or shooting, stinging, burning, and/or radiating sensations throughout the back or extremities, including, but not limited to, the subject's head, shoulders, arms, legs, and/or back. Neck pain may be idiopathic or associated with one or more (diagnosed or undiagnosed) underlying conditions, including, but not limited to, degenerative disc disease, rheumatoid arthritis, osteoporosis, fibromyalgia, chronic inflammation, infection (e.g., discospondylitis), herniated disc, spondylosis, spinal stenosis, cervical compressive myelopathy, whiplash, and/or other disorders.

The terms “polynucleotide,” “nucleotide,” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA, tRNA, lncRNA, RNA antagomirs, and inhibitory DNA or RNA (RNAi, e.g., small or short hairpin (sh)RNA, microRNA (miRNA), aptamers, small or short interfering (si)RNA, trans-splicing RNA, or antisense RNA). Polynucleotides also include non-coding RNA, which include for example, but are not limited to, RNAi, miRNAs, lncRNAs, RNA antagomirs, aptamers, and any other non-coding RNAs known to those of skill in the art. Polynucleotides include naturally occurring, synthetic, and intentionally altered or modified polynucleotides as well as analogues and derivatives. The term “polynucleotide” also refers to a polymeric form of nucleotides of any length, including deoxyribonucleotides or ribonucleotides, or analogs thereof, and is synonymous with nucleic acid sequence. A polynucleotide may comprise modified nucleotides, such as methylated nucleotides and nucleotide analogs, and may be interrupted by non-nucleotide components. If present, modifications to the nucleotide structure may be imparted before or after assembly of the polymer. The term polynucleotide, as used herein, refers interchangeably to double- and single-stranded molecules. Unless otherwise specified or required, any embodiment as described herein encompassing a polynucleotide encompasses both the double-stranded form and each of two complementary single-stranded forms known or predicted to make up the double-stranded form. Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

The term “gene” or “nucleotide sequence encoding a polypeptide” refers to the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons). For example, a gene includes a polynucleotide containing at least one open reading frame capable of encoding a particular protein or polypeptide after being transcribed and translated.

The terms “extracellular domain” and “ectodomain” may be used interchangeably and, when referring to transmembrane cellular receptors, is defined as the portion of the protein that is exposed to the extracellular environment and is able to engage with and/or bind a ligand.

The terms “cytoplasmic domain” and “intracellular domain” may be used interchangeably and, when referring to transmembrane receptors, define the portion of the protein that is exposed to the cytoplasm. In many instances, these portions of the proteins comprise signaling domains to recruit and associate with various intracellular factors. Following engagement with a ligand via the extracellular domain, the interaction effects changes that may result in new association, dissociation or recruitment of various cytoplasmic factors that aid in transducing a signal.

The term “transmembrane domain,” which may be abbreviated as “TM,” as it refers to transmembrane receptors, is defined as the portion of the protein is embedded within the plasma membrane (i.e., not exposed to either the extracellular environment or the cytosol). Transmembrane domains are generally of a more hydrophobic character than either the extracellular or cytoplasmic portions and often adopt higher order helical structures. Though its primary role is an anchor, ligand-induced conformational changes to particular receptors have been shown to impact the transmembrane domain such that it is integral to the subsequent intracellular signaling.

The term “receptor” refers to a protein capable of binding another cognate protein (i.e., its ligand) with high affinity. This receptor-ligand interaction may be 1:1, or result in multimerization, wherein numerous proteins aggregate to bind one or more ligands. Receptors are generally present at the cell surface, such that they may most efficiently encounter a ligand and initiate intracellular signaling.

The term “intracellular signaling” refers to cellular changes that result due to events occurring at the cell surface. Typically, a soluble ligand binds its receptor at the cell surface, which can induce changes in the receptor, such that associated intracellular factors are also affected. These factors may then impact others within the cell, and this cascade continues until, in many cases, a particular factor is able to alter gene expression in the nucleus in response to the stimulus at the surface.

The term “RNA-guided nuclease” refers to an enzyme capable of breaking the backbone of, for example, a DNA molecule. The activity of RNA-guided nucleases is directed by a nucleic acid molecule (i.e., guide RNA). Once properly oriented to form a functional ribonucleoprotein complex, the enzyme locates a specific position within a target nucleic acid (e.g., a gene or locus) via sequence complementarity with a portion of the guide RNA. Non-exhaustive examples of RNA-guided nucleases include Cas9, Cas12 and Cas12a (previously known as Cpf1).

The term “Cas9” refers to an RNA-guided, double-stranded DNA-binding nuclease protein or nickase protein, or a variant thereof and may be used to refer to either naturally-occurring or recombinant Cas9 nucleases variants (e.g., ES-Cas9, HF-Cas9, PE-Cas9, and AR-Cas9). The wildtype Cas9 nuclease has two functional domains, e.g., RuvC and HNH, that simultaneously cut both strands of double stranded DNA, resulting in a double-strand break. Cas9 enzymes described herein may comprise a HNH or HNH-like nuclease domain and/or a RuvC or RuvC-like nuclease domain without impacts on the ability to induce double-strand breaks in genomic DNA (e.g., at a target locus) when both functional domains are active. The Cas9 enzyme may comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

As used herein, “PAM” refers to a Protospacer Adjacent Motif and is necessary for an RNA-guided nuclease to bind a target nucleic acid. In many instances, the PAM directly abuts the complementary sequence in the target. Naturally occurring Cas9, for example, molecules recognize specific PAM sequences (see, e.g., Table 1). In some embodiments, a Cas9 molecule has the same PAM specificities as a naturally occurring Cas9 molecule. In other embodiments, a Cas9 molecule has a PAM specificity not associated with a naturally occurring Cas9 molecule. In other embodiments, a Cas9 molecule's PAM specificity is not associated with the naturally occurring Cas9 molecule to which it has the closest sequence homology. For example, a naturally occurring Cas9 molecule can be altered such that the PAM sequence recognition is altered to decrease off target sites, improve specificity, or eliminate a PAM recognition requirement. In an embodiment, a Cas9 molecule may be altered (e.g., to lengthen a PAM recognition sequence, improve Cas9 specificity to high level of identity, to decrease off target sites, and/or increase specificity). In an embodiment, the length of the PAM recognition sequence is at least 4, 5, 6, 7, 8, 9, 10 or 15 amino acids in length. In some embodiments, a Cas9 molecule may be altered to ablate PAM recognition.

The terms “guide RNA,” “gRNA” or “sgRNA” may be used interchangeably and refer to an RNA molecule, preferably a synthetic RNA molecule, composed of a targeting (crRNA) sequence and scaffold. These molecules, once loaded onto a functional RNA-guided nuclease can direct sequence-specific cleavage of a target nucleic acid.

An sgRNA can be administered or formulated, e.g., as a synthetic RNA, or as a nucleic acid comprising a sequence encoding the gRNA, which is then expressed in the target cells. As would be evident to one of ordinary skill in the art, various tools may be used in the design and/or optimization of an sgRNA in order to, for example, increase specificity and/or precision of genomic editing at a particular site.

In general, candidate sgRNAs may be designed and identified by first locating suitable PAMs within a genomic sequence. Then additional calculations may be utilized to predict on-target and off-target efficiencies. Available web-based tools to aid in the initial design and modeling of candidate sgRNAs include, without limitation, CRISPRseek, CRISPR Design Tool, Cas-OFFinder, E-CRISP, ChopChop, CasOT, CRISPR direct, CRISPOR, BREAKING-CAS, CrispRGold, and CCTop. See, e.g., Safari, F. et al. (2017). Current Pharmaceutical Biotechnology, 18(13):1038-54, which is incorporated by reference herein in its entirety for all purposes. Such tools are also described, for example, in PCT Publication No. WO2014093701A1 and Liu, G. et al. (2020). Computational approaches for effective CRISPR guide RNA design and evaluation. Computational and Structural Biotechnology Journal, 18: 35-44, each of which is incorporated by reference herein in its entirety for all purposes. Candidate sgRNAs may be further assessed by experimental screening or other methodologies.

The terms “CRISPR RNA” or “crRNA” refer to the portion of an sgRNA molecule with complementarity to the target nucleic acid.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the disclosure is contemplated. Additional active pharmaceutical ingredients, such as other drugs, can also be incorporated into the described compositions and methods.

The term “pharmaceutically acceptable excipient” is intended to include vehicles and carriers capable of being co-administered with a compound to facilitate the performance of its intended function. The use of such media for pharmaceutically active substances is well known in the art. Examples of such vehicles and carriers include solutions, solvents, dispersion media, delay agents, emulsions and the like. Any other conventional carrier suitable for use with the multi-binding compounds also falls within the scope of the present disclosure.

As used herein, the term “a”, “an”, or “the” generally is construed to cover both the singular and the plural forms.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that compositions, amounts, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate,” whether or not expressly stated to be such. It is noted that embodiments of very different sizes, shapes and dimensions may employ the described arrangements.

The term “substantially” as used herein can refer to a majority of, or mostly, as in at least about 50%, 60%, 70%, 80%, 90%, 95%, 96%, 97%, 98%, 99%, 99.5%, 99.9%, 99.99%, or at least about 99.999% or more.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed methods and compositions. All compositions, methods, and kits described herein that embody the present disclosure can, in alternate embodiments, be more specifically defined by any of the transitional terms “comprising,” “consisting essentially of,” and “consisting of.”

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, subcutaneous, intraarticular, or intradiscal route). Administration of a nanoparticle composition to a mammal or mammalian cell may involve contacting one or more cells with the nanoparticle composition.

As used herein, “naturally occurring” means existing in nature without artificial aid.

As used herein, a “PEG lipid” or “PEGylated lipid” refers to a lipid comprising a polyethylene glycol component. These lipids may also be referred to a PEG-modified lipids.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

Any of the compositions disclosed herein can be administered to a non-human subject, such as a laboratory or farm animal. Non-limiting examples of a non-human subject include laboratory or research animals, pets, wild or domestic animals, farm animals, etc., e.g., a dog, a goat, a guinea pig, a hamster, a mouse, a pig, a non-human primate (e.g., a gorilla, an ape, an orangutan, a lemur, a baboon, etc.), a rat, a sheep, a horse, a cow, or the like. As used herein, a “lipid component” is that component of a nanoparticle composition that includes one or more lipids. For example, the lipid component may include one or more cationic/ionizable, PEGylated, structural, or other lipids, such as phospholipids.

### III. Methods

### A. CRISPR

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In one aspect, the present disclosure encompasses compositions relating to clustered regularly interspaced short palindromic repeats (CRISPR) and CRISPR-associated RNA-guided nucleases and associated methods, components, and compositions (hereafter, CRISPR/Cas systems). Such systems minimally require at least one isolated or non-naturally occurring RNA-guided nuclease (e.g., a Cas9 protein) and at least one isolated or non-naturally occurring guide RNA (e.g., an sgRNA) to effectuate augmentation of a nucleic acid sequence (e.g., genomic DNA).

In some embodiments, a CRISPR/Cas system effectuates the alteration of a targeted gene or locus in a eukaryotic cell by effecting an alteration of the sequence at a target position (e.g., by creating an insertion or deletion (collectively, an indel) resulting in loss-of-function of (i.e., knocking out) the affected gene or allele; e.g., a nucleotide substitution resulting in a truncation, nonsense mutation, or other type of loss-of-function of an encoded product of, for example, (i) one or more growth factors or growth factor receptors (e.g., FGF2, CCN2, NGF, NTF3, NTF4, BDNF, FGFR1, NGFR, NTRK1, NTRK2), (ii) one or more metallproteases or regulators thereof (e.g., ADAM17, ADAMTS1, ADAMTS5, MMP1, MMP2, MMP3, MMP7, MMP8, MMP10, MMP12, MMP13, TIMP1, TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, ATP1A1), (v) one or more other regulators of cell signaling (e.g., CALCA, CALCB, CALCRL, RAMP1, ADM, CRCP, YAP1, MRGPRX2), and (vi) combinations of any genes of (i)-(v), i.e., mRNA or protein; a deletion of one or more nucleotides resulting in a truncation, nonsense mutation, or other type of loss-of-function of an encoded product of, for example, one or more FGF2, CCN2, ADAMTS5, MMP1, or NGF gene; e.g., loss-of-function of the encoded mRNA or protein by a single nucleotide, double nucleotide, or other frame-shifting deletion, or a deletion resulting in a premature stop codon; or an insertion resulting in a truncation, nonsense mutation, or other type of loss-of-function of an encoded gene product, such as an encoded gene product of, for example, one or FGF2, CCN2, ADAMTS5, MMP1, or NGF gene (i.e., mRNA or protein); e.g., a single nucleotide, double nucleotide, or other frame-shifting insertions, or an insertion resulting in a premature stop codon. In some embodiments, a CRISPR/Cas system of the present disclosure provides for the alteration of a gene and/or encoded product of a gene, such that the altered product has a resultant loss-of-function and becomes a dominant negative or decoy (e.g., a transmembrane receptor incapable of initiating intracellular signaling or a soluble receptor). In some embodiments, a CRISPR/Cas system of the present disclosure is encapsulated in an LNP system described herein, e.g., and without limitation, any one of LNP systems LNP001 to LNP240.

In one aspect, CRISPR/Cas systems effectuate changes to the sequence of a nucleic acid through nuclease activity. For example, in the case of genomic DNA, the RNA-guided-nuclease locates a target position within a targeted gene or locus by sequence complementarity with the target genomic sequence (e.g., CRISPR RNA (crRNA) or a complementary component of a synthetic single guide RNA (sgRNA)) and cleaves the genomic DNA upon recognition of a particular, nuclease-specific motif called the protospacer adjacent motif (PAM). See generally, Collias, D., & Beisel, C. L. (2021). Nature Communications, 12(1), 1-12.

Nuclease activity (i.e., cleavage) induces a double-strand break (DSB) in the case of genomic DNA. Endogenous cellular mechanisms of DSB repair, namely non-homologous end joining (NHEJ), microhomology-mediated end joining (MMEJ), and homologous recombination, result in erroneous repair at a given target position with some calculable frequency as a result of interference from said components of the CRISPR/Cas system, thereby introducing substitutions or indels into the genomic DNA. See generally Scully, R., et al. (2019). Nature Reviews Molecular Cell Biology, 20(11), 698-714. At some frequency, these indels and/or substitutions may result in frameshifts, nonsense mutations (i.e., early stop codons) or truncations that impact the availability of gene products, such as mRNA and/or protein. In certain embodiments, the CRISPR/Cas system may induce a homology-directed repair (HDR) mechanism leading to insertions of non-random sequences at a target position through the use of templates (e.g., an HDR template) provided to the cell as part of the system along with the nuclease and gRNA. See Bloh, K., & Rivera-Torres, N. (2021). International Journal of Molecular Sciences, 22(8), 3834.

In general, the minimum requirements of the CRISPR/Cas system will be dependent upon the nuclease (i.e., Cas protein) provided therewith. To this extent, these bacterially derived nucleases have been functionally divided into Types I, III, and V, which all fall into Class 1 and Types II, IV, and VI that are grouped into Class 2.

### Class 1 CRISPR/Cas Systems:

The exact components, compositions, and methods for effectuating a change in a targeted nucleic acid sequence using a Class 1 CRISPR/Cas system will vary, but should minimally include: a nuclease (selected from at least Types I, and III), at least one guide RNA selected from 1) sgRNA or 2) a combination of crRNA and tracrRNA. These CRISPR/Cas systems have been categorized together as Class 1 CRISPR/Cas systems due to their similarities in requirements and mode of action within a eukaryotic cell. To this end, compositions, components, and methods among Class 1 constituents may be considered functionally interchangeable, and the following details, provided merely for exemplary purposes, do not represent an exhaustive list of class members. In some embodiments, a CRISPR/Cas system of the present disclosure is encapsulated in an LNP system described herein, e.g., and without limitation, any one of LNP systems LNP001 to LNP240.

Cas3 (see Table 1) is the prototypical Type I DNA nuclease that functions as the effector protein as part of a larger complex (the Cascade complex comprising Cse1, Cse2,), that is capable of genome editing. See generally He, L., et al. (2020). Genes, 11(2), 208. Unlike other CRISPR/Cas systems, Type I systems localize to the DNA target without the Cas3 nuclease via the Cascade complex, which then recruits Cas3 to cleave DNA upon binding and locating the 3′ PAM. The Cascade complex is also responsible for processing crRNAs such that they can be used to guide it to the target position. Because of this functionality, Cascade has the ability to process multiple arrayed crRNAs from a single molecule. See. Luo, M. (2015). Nucleic Acids Research, 43(1), 674-681. As such, Type I system may be used to edit multiple targeted genes or loci from a single molecule.

Because the natural Cas3 substrate is ssDNA, its function in genomic editing is thought to be as a nickase; however, when targeted in tandem, the resulting edit is a result of blunt end cuts to opposing strands to approximate a blunt-cutting endonuclease, such as Cas9. See Pickar-Oliver, A., & Gersbach, C. A. (2019). Nature Reviews Molecular Cell Biology, 20(8), 490-507.

Like Type I nucleases, the Type III system relies upon a complex of proteins to effect nucleic acid cleavage. Particularly, Cas10 possesses the nuclease activity to cleave ssDNA in prokaryotes. See Tamulaitis, G. Trends in Microbiology, 25(1), 49-61 (2017). Interestingly, this CRISPR/Cas system, native to archaea, exhibits dual specificity and targets both ssDNA and ssRNA. Aside from this change, the system functions much like Type I in that the crRNA targets an effector complex (similar to Cascade) in a sequence-dependent manner. Similarly, the effector complex processes crRNAs prior to association. The dual nature of this nuclease makes its applications to genomic editing potentially more powerful, as both genomic DNA and, in some cases, mRNAs with the same sequence may be targeted to silence particular targeted genes.

### Class 2 CRISPR/Cas Systems:

The exact components, compositions, and methods for effectuating a change in a targeted nucleic acid sequence using a Class 2 CRISPR/Cas system will vary but should minimally include: a nuclease (selected from at least Types II, and V), at least one guide RNA selected from 1) sgRNA or 2) a combination of crRNA and tracrRNA. These CRISPR/Cas systems have been categorized together as Class 2 CRISPR/Cas systems due to their similarities in requirements and mode of action within a eukaryotic cell. To this end, compositions, components, and methods among Class 2 constituents may be considered functionally interchangeable, and the following details, provided merely for exemplary purposes, do not represent an exhaustive list of class members:

Type II nucleases are the best-characterized CRISPR/Cas systems, particularly the canonical genomic editing nuclease Cas9 (see Table 1). Multiple Cas9 proteins, derived from various bacterial species, have been isolated. The primary distinction between these nucleases is the PAM, a required recognition site within the targeted dsDNA. After association with a gRNA molecule, the crRNA (or targeting domain of a sgRNA) orients the nuclease at the proper position, but the protein's recognition of the PAM is what induces a cleavage event near that site, resulting in a blunt DSB.

In addition to the naturally derived Cas9 proteins, several engineered variants have similarly been reported. These range from Cas9 with enhanced specific (i.e., less off-target activity), such as espCas9. Others have been catalytically modified via point mutations in the RuvC (e.g., D10A) and HNH (e.g., H840A) domains such that they induce only single-strand breaks (i.e., Cas9 nickases). See Frock, R. et al. (2015). Nature Biotechnology, 33(2), 179-186. These have also been shown to be less error-prone in editing. Such mitigation of off-target effects becomes paramount when selecting for a desired insertion (i.e., a knock in mutation, in which a desired nucleotide sequence is introduced into a target nucleic acid molecule) rather than a deletion. Indeed, less off-target effects may aid in the preferred DNA repair mechanism (HDR, in most instances for knock in mutations). See generally Naeem, M., et al. (2020). Cells, 9(7), 1608.

Additional exemplary further engineered variants of canonical Cas proteins (e.g., mutants, chimeras, and include the following (each of which are hereby incorporated by reference in their entireties for all purposes): WO2015035162A2, WO2019126716A1, WO2019126774A1, WO2014093694A1, WO2014150624A1, US20190225955A1, U.S. patent Ser. No. 11/427,818, U.S. patent Ser. No. 11/242,542, U.S. patent Ser. No. 11/098,297, U.S. patent Ser. No. 10/876,100, U.S. patent Ser. No. 10/767,193, U.S. patent Ser. No. 10/494,621, and U.S. patent Ser. No. 10/100,291.

For the avoidance of doubt, SpCas9 collectively refers to any one of the group consisting of espCas9 (also referred to herein as ES-Cas9 or esCas9), HF-Cas9, PE-Cas9, ARCas9 (also referred to as AR-Cas9), SpCas9-D1135E, SpCas9-HF1, HypaCas9, HiFiCas9, xCas9-3.6, xCas9-3.7, Sniper-Cas9, evoCas9, SpartaCas, LZ3Cas9, miCas9, and SuperFi-Cas9. Additional examples of Cas9 variants disclosed in the following are hereby incorporated by reference in their entireties for all purposes: Huang, X., et al. (2022). Cells, 11(14), 2186.

Like the canonical Cas9 systems, Type V nucleases only require a synthetic sgRNA with a targeting domain complementary to a genomic sequence to carry out genomic editing. These nucleases contain a RuvC domain but lack the HNH domain of Type II nucleases. Further, Cas12, for example, leaves a staggered cut in the dsDNA substrate distal to the PAM, as compared to Cas9's blunt cut next to the PAM. Both Cas12a, also known as Cpf1, and Cas12b, also known as C2cl (see Table 1), act as part of larger complex of two gRNA-associated nucleases that acts on dsDNA as a quaternary structure, nicking each strand simultaneously. See Zetsche, B. et al. (2015). Cell, 163(3):759-771; see also Liu, L. et al. (2017). Molecular Cell, 65(2):310-322. Additionally, Cas12b (C2cl) is a highly accurate nuclease with little tolerance for mismatches. See Yang, H. et al. (2016). Cell, 167(7):1814-1828.e12.

TABLE 1
Exemplary list of Cas nucleases and their requirements

| | | | Spacer |
|---|---|---|---|
| | PAM | Type of end generated | length |
| Nuclease (Species) | (5′→3′) | (nucleic acid target) | (nt) |
| Cas9 (S. pyogenes) | NGG | Blunt (dsDNA) | 20 |
| Cas9 (S. aureus) | NNGRRT | Blunt (dsDNA) | 20 |
| Cas9 (C. jejuni) | NNNNRYAC | Blunt (dsDNA) | 22 |
| Cas9 (S. thermophilus) | NNAGAAW | Blunt (dsDNA) | 20 |
| Cas9 (N. meningitidis) | NNNNGATT | Blunt (dsDNA) | 24 |
| Cas9 (F. novicida) | NGG | Blunt (dsDNA) | 21 |
| Cas12a (L. bacterium) | TTTV | 5′ staggered | 23-25 |
| | | (dsDNA/ssDNA) | |
| Cas12a (Acidaminococcus | TTTN | 5′ staggered | 24 |
| sp.) | | (dsDNA/ssDNA) | |
| Cas3 (E. coli) | CTT/CCT/CAT/CTC | None/blunt (ssDNA) | 32 |

See generally Wang, J., Zhang, C., & Feng, B. (2020). Journal of Cellular and Molecular Medicine, 24(6), 3256-3270, where N = any nucleotide; R = any purine (A or G); Y = any pyrimidine (C or T); W = A or T ; V = A, C or G.

In one aspect, the CRISPR/Cas system of the present disclosure comprises at least one RNA-guided nuclease (e.g. a Cas protein) derived from one or more of the following selected bacterial genera: Corynebacterium, Sutterella, Legionella, Treponema, Fiifactor, Eubacterium, Streptococcus, Lactobacillus, Mycoplasma, Bacteroides, Flavobacterium, Spirochaeta, Azospirillum, Gluconacetobacter, Neisseria, Roseburia, Parvibaculum, Nitratifractor, Campylobacter, Pseudomonas, Streptomyces, Staphylococcus, Francisella, Acidaminococcus, Lachnospiraceae, Leptotrichia, and Prevolella. In some embodiments, the Cas protein is derived from Deltaproteobacteria or Planctomycetes bacterial species.

Some aspects of the present disclosure provide strategies, methods, compositions, and treatment modalities for altering a targeted sequence within a gene locus (e.g., altering the sequence of wild type and/or of a mutant sequence within a cell or within a mammal) by insertion or deletion of one or more nucleotides mediated by an RNA-guided nuclease and one or more guide RNAs (gRNAs), resulting in loss of function of the targeted gene product. In some embodiments, the loss of function results in “knocking out” the gene of interest (i.e., generation of a “knock out”) by ablating gene expression. In some embodiments, the loss function results in a non-functional gene product (i.e., a gene product without all functionality of the wildtype gene product). In some embodiments, the loss of function results in expression of gene product with different characteristics (e.g., different binding affinity or different cellular localization).

In certain embodiments, the targeted gene is selected from (i) one or more growth factors or growth factor receptors (e.g., FGF2, CCN2, NGF, NTF3, NTF4, BDNF, FGFR1, NGFR, NTRK1, NTRK2), (ii) one or more metallproteases or regulators thereof (e.g., ADAM17, ADAMTS1, ADAMTS5, MMP1, MMP2, MMP3, MMP7, MMP8, MMP10, MMP12, MMP13, TIMP1, TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, ATP1A1), (v) one or more other regulators of cell signaling (e.g., CALCA, CALCB, CALCRL, RAMP1, ADM, CRCP, YAP1, MRGPRX2), and (vi) combinations of any genes of (i)-(v). In some embodiments, any region of the targeted gene (e.g., a promoter region, a 5′ untranslated region, a 3′ untranslated region, an exon, an intron, or an exon/intron border) is targeted by an RNA-guided nuclease to alter the gene. In some embodiments, a non-coding region of the targeted gene (e.g., an enhancer region, a promoter region, an intron, 5′ UTR, 3′ UTR, polyadenylation signal) is targeted to alter the gene.

### CRISPR Guide RNAs:

In one aspect, the CRISPR/Cas system of the present disclosure further provides a gRNA molecule (e.g., an isolated or non-naturally occurring RNA molecule) that interacts with the RNA-guided nuclease. In certain embodiments, the gRNA is an sgRNA comprising a crRNA sequence comprising a nucleotide sequence which is complementary to a sequence in a target nucleic acid. In some embodiments, the sgRNA further comprises an RNA scaffolding portion (i.e., tracrRNA) that interacts with the RNA-guided nuclease, such that the crRNA is positioned to scan a target nucleic acid for complementarity. In some embodiments, the system is further, optionally, comprised of an oligonucleotide—an HDR template with homology to either side of the target position. See Bloh, K., & Rivera-Torres, N. (2021). International Journal of Molecular Sciences, 22(8):3834.

In an embodiment, the RNA-guided nuclease and sgRNA are configured to orient an associated nuclease such that a cleavage event, (e.g., a double strand break or a single strand break) occurs sufficiently close to a complementary sequence in the targeted nucleic acid, thereby facilitating an alteration in the nucleic acid sequence. In some embodiments, the crRNA is 20 nucleotides in length. In some embodiments, the crRNA is 14, 15, 16, 17, 18, 19, 20, 21, 22, 23, 24, 25, 26, 27, 28, 29, or 30 nucleotides in length.

In some embodiments, the crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In certain embodiments, a second gRNA molecule, comprising a second crRNA orients a second RNA-guided nuclease, such that a cleavage event occurs sufficiently close to a complementary sequence in the targeted nucleic acid, thereby facilitating an alteration in the nucleic acid sequence. In some embodiments, the first gRNA and the second gRNA promote a cleavage event within a single targeted gene. In some embodiments, the first gRNA and the second gRNA promote a cleavage event within different targeted genes. In some embodiments, the second crRNA is 20 nucleotides in length. In some embodiments, the second crRNA is 14, 15, 16, 17, 18, 19, 20, 21, 22, 23, 24, 25, 26, 27, 28, 29, or 30 nucleotides in length.

In some embodiments, the second crRNA orients the RNA-guided nuclease such that a cleavage event occurs within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides away from the complementary sequence in the targeted nucleic acid. The double- or single-strand break may be positioned upstream or downstream of the complementary sequence in the targeted nucleic acid. In some embodiments, the cleavage event occurs within a targeted gene. In some embodiments, the cleavage event occurs upstream of a targeted gene.

In some embodiments, the targeting domains of the first gRNA and the second gRNA are configured such that a cleavage event is positioned, independently for each of the gRNA molecules, within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of the others cleavage event. In some embodiments, the first gRNA and the second gRNA molecules alter the targeted nucleic acid sequences simultaneously. In some embodiments, the first gRNA and the second gRNA molecules alter the targeted nucleic acid sequences sequentially.

In some embodiments, a single-strand break is accompanied by a second single-strand break, positioned by the crRNA of a first gRNA and a second gRNA, respectively. For example, the crRNA may orient the associated RNA-guided nucleases such that a cleavage event, (e.g., the two single-strand breaks), are positioned within 1, 2, 3, 4, 5, 10, 15, 20, 25, 30, 35, 40, 45, 50, 55, 60, 65, 70, 75, 80, 85, 90, 95, 100, 125, 150, 175, or 200 nucleotides of one another. In some embodiments, a first crRNA and a second crRNA are configured to orient associated RNA-guided nucleases such that, for example, two single-strand breaks occur at the same position, or within 1, 2, 3, 4, 5, 10, 15, 20, 25, 30, 35, 40, 45, 50 nucleotides of one another, on opposing strands of genomic DNA, thereby essentially approximating a double strand break.

In some embodiments a nucleic acid encodes a second sgRNA molecule. In some embodiments, a nucleic acid encodes a third sgRNA molecule. In some embodiments, a nucleic acid encodes a fourth sgRNA molecule.

In certain embodiments, a nucleic acid may comprise (a) a sequence encoding a first sgRNA, comprising a crRNA that is complementary with a sequence in a targeted gene, (b) a sequence encoding a second sgRNA, comprising a crRNA that is complementary with a sequence in a second targeted gene, and (c) a sequence encoding an RNA-guided nuclease (e.g., Cas9). Optionally, (d) and (e) are sequences encoding a third sgRNA and a fourth sgRNA, respectively. In some embodiments, the second targeted gene is the same as the first targeted gene. In other embodiments, the second targeted gene is different from the first targeted gene. In some embodiments, (a), (b), and (c) are encoded within the same nucleic acid molecule (e.g., the same vector). In some embodiments, (a) and (b) are encoded within the same nucleic acid molecule. In some embodiments, (a), (b) and (d) are encoded within the same nucleic acid molecule. In some embodiments, (a), (b) and (e) are encoded within the same nucleic acid molecule. In some embodiments, (a), (b), (d) and (e) are encoded within the same nucleic acid molecule. In some embodiments, (a), (b), and (c) are encoded within separate nucleic acid molecules. When more than two sgRNAs are used, any combination of (a), (b), (c), (d) and (e) may be encoded within a single or separate nucleic acid molecules.

In one aspect, the nucleic acid molecules (i.e., those encoding (a), (b), (c), (d) or (e)) are delivered to a target cell (i.e., any combination of the encoded RNA-guided nuclease of (c) and at least one encoded gRNA molecule of (a), (b), (d), or (e) contact a target cell). In some embodiments, said nucleic acid molecules are delivered to a target cell in vivo. In other embodiments, said nucleic acid molecules are delivered to a target cell ex vivo. In some embodiments, said nucleic acid molecules are delivered to a target cell in vitro. In certain embodiments, said nucleic acid molecules are delivered to a target cell as DNA. In other embodiments, said nucleic acid molecules are delivered to a target cell as RNA (e.g., mRNA). In some embodiments, the products of said nucleic acid molecules are delivered as an assembled ribonucleoprotein (RNP).

In some embodiments, contacting a target cell comprises delivering said RNA-guided nuclease of (c), as a protein with at least one said nucleic acid molecules selected from (a), (b), (d), and (e). In some embodiments, contacting a target cell comprises delivering said encoded RNA-guided nuclease of (c), as DNA with at least one said nucleic acid molecules selected from (a), (b), (d), and (e). In some embodiments, contacting a target cell comprises delivering said encoded RNA-guided nuclease of (c), as mRNA with at least one said nucleic acid molecules selected from (a), (b), (d), and (e).

In certain embodiments, CRISPR components are delivered to a target cell via nanoparticles. Exemplary nanoparticles that may be used with all CRISPR/Cas systems disclosed herein include, at least, lipid nanoparticles or liposomes, hydrogel nanoparticles, metalorganic nanoparticles, gold nanoparticles, magnetic nanoparticles and virus-like particles. See generally Xu, C. F. et al. (2021). Advanced Drug Delivery Reviews, 168:3-29. In some embodiments, CRISPR components of the present disclosure are encapsulated in an LNP system described herein, e.g., and without limitation, any one of LNP systems LNP001 to LNP240.

### B. TALEN

In one aspect, the present disclosure contemplates use of methods, components, and compositions relating to Transcription Activator-Like Effector Nucleases (TALENs) to effectuate augmentation of a ‘nucleic acid sequence (e.g., a targeted gene.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present disclosure are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

Non-limiting examples of genes that may be silenced or inhibited by permanently gene-editing via a TALE method include (i) one or more growth factors or growth factor receptors (e.g., FGF2, CCN2, NGF, NTF3, NTF4, BDNF, FGFR1, NGFR, NTRK1, NTRK2), (ii) one or more metallproteases or regulators thereof (e.g., ADAM17, ADAMTS1, ADAMTS5, MMP1, MMP2, MMP3, MMP7, MMP8, MMP10, MMP12, MMP13, TIMP1, TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, ATP1A1), (v) one or more other regulators of cell signaling (e.g., CALCA, CALCB, CALCRL, RAMP1, ADM, CRCP, YAP1, MRGPRX2), and (vi) combinations of any genes of (i)-(v). In an aspect, the disclosure provides compositions for up-regulation of protein receptors (including wildtype or genetically edited), including those that bind to anti-inflammatory cytokines via a TALE method.

Examples of systems, methods, and compositions for altering the expression of a target gene sequence by a TALE method, and which may be used in accordance with embodiments of the present disclosure, are described in U.S. Pat. No. 8,586,526, which is incorporated by reference herein.

### C. Zinc-Finger Nucleases (ZFN)

In one aspect, the present disclosure contemplates use of methods, components, and compositions relating to zinc-finger nucleases (ZFNs) to effectuate augmentation of a ‘nucleic acid sequence (e.g., a targeted gene).

An individual zinc finger contains approximately 30 amino acids in a conserved ββα configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, MO, USA).

Non-limiting examples of genes that may be silenced or inhibited by permanently gene-editing via a zinc finger method include (i) one or more growth factors or growth factor receptors (e.g., FGF2, CCN2, NGF, NTF3, NTF4, BDNF, FGFR1, NGFR, NTRK11, NTRK2), (ii) one or more metallproteases or regulators thereof (e.g., ADAM17, ADAMTS1, ADAMTS5, MMP1, MMP2, MMP3, MMP7, MMP8, MMP10, MMP12, MMP13, TIMP1, TIMP3), (iii) one or more cytokines, chemokines or cytokine/chemokine receptors (e.g., CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL10RA, IL10RB, IL13RA1, IL13RA2, IL17RA, IL18R1, IL18RAP), (iv) one or more regulators of neuronal signaling (e.g., SCN1A, SCN2A, SCN3A, SCN4A, SCN5A, SCN8A, SCN9A, SCN10A, SCN11A, TAC1, TAC3, TACR1, TACR2, TACR3, ATP1A1), (v) one or more other regulators of cell signaling (e.g., CALCA, CALCB, CALCRL, RAMP1, ADM, CRCP, YAP1, MRGPRX2), and (vi) combinations of any genes of (i)-(v). Non-limiting examples of genes that may be augmented such that their resultant products function as decoys or dominant negatives by permanently gene-editing via a zinc finger method include. In an aspect, the disclosure provides compositions for up-regulation of protein receptors (including wildtype or genetically edited), including those that bind to anti-inflammatory cytokines via a zinc finger method.

Examples of systems, methods, and compositions for altering the expression of a target gene sequence by a zinc finger method, which may be used in accordance with embodiments of the present disclosure, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present disclosure, are described in Beane, et al., Mol. Therapy, 2015, 23 1380-1390, the disclosure of which is incorporated by reference herein.

### IV. Joint Disease or Illness

### A. Introduction

As described herein, embodiments of the present disclosure provide compositions and methods for improving joint function and treating joint disease. In particular embodiments, compositions and methods are provided to gene-edit synovial fibroblasts, synoviocytes, chondrocytes, tissue (resident) macrophages, or other cells to reduce pro-inflammatory signaling mediated by the binding of inflammatory cytokines-including, but not limited to, IL1α, IL1β, TNFα, IL6, IL8, IL18, IL33, matrix metalloproteinases (MMPs), TGFβ1, TGFβ2, and combinations thereof—to their cognate receptor(s). Some embodiments are used for treating various forms of arthritis and other inflammatory joint diseases. Some embodiments are further useful for treating canine lameness due to osteoarthritis. Some embodiments are further useful for treating equine lameness due to joint disease. Some embodiments are further useful for treating feline lameness due to joint disease. Some embodiments are also useful for treating post-traumatic arthritis, gout, pseudogout, psoriatic arthritis, and other inflammation-mediated or immune-mediated joint diseases. Some embodiments are further useful as it relates to encapsulation in an LNP system described herein, e.g., and without limitation, any one of LNP systems LNP001 to LNP240.

Treatment of osteoarthritis, degenerative joint disease, and other joint dysfunctions is complex, and few long-term options exist for either symptomatic relief or restoring joint function. Osteoarthritis (OA) is the leading cause of disability due to pain. See, Neogi, T. (2013). Osteoarthritis Cartilage, 21(9):1145-53. OA and similar diseases impact all mammal species, including working animals, domestic pets, and their owners. The common mechanistic thread among joint diseases is the presence of acute of chronic inflammation, which is driven by increased levels of pro-inflammatory cytokine signaling. Joint diseases tend to take a progressive course that encompasses discomfort, pain, and—especially in the case of OA—disability, depending on the degree of disease progression.

Psoriatic arthritis (PsA) is another chronic inflammatory joint disease, in which the joint symptoms are accompanied by skin lesions, such as those commonly associated with psoriasis. See, Boehncke, W. et al. (2014). British Journal of Dermatology, 170(4):772-786. Like other forms of arthritis, such as OA, PsA is caused by pro-inflammatory signaling of a host of cytokines, including IL1. Indeed, PsA morbidity has been shown to correlate with single nucleotide polymorphisms (SNPs) that impact the activity of the IL1 gene locus. See, Rahman, P. et al. (2006). Arthritis and Rheumatism, 54(7):2321-2325. These studies also implicate inflammatory cytokine signaling, in general, and IL1 more specifically, in disease progression.

Gout is a chronic inflammatory condition that affects joints. The underlying cause is monosodium urate (MSU) crystal deposition and the resultant host response, particularly in joint structures (as well as subcutaneous tissues and other sites). See, Dalbeth, N., & Stamp, L. (2014). Annals of the Rheumatic Diseases, 73(9):1598-1600. The clinical manifestations include recurrent acute flares of severe inflammatory arthritis and tendinobursitis. IL1 and other pro-inflammotory mediators are a major contributor to this host response. See, Dinarello, C. A. (2014). Molecular Medicine, 20(1):S43-S58. To this end, effective blockade of these signaling pathways may provide relief to gout patients.

The current standard of care for many joint disease patients includes anti-inflammatory medications (e.g., NSAIDs) or anti-rheumatics (e.g., methotrexate [inhibitor of AICAR] or adalimumab [anti-TNF alpha monoclonal antibody]). See, Friedman, B., & Cronstein, B. (2019). Joint Bone Spine, 86(3):301-307. All of these treatments require repeated dosing for continued effectiveness, which may lead to toxicity issues or tolerance over time. As such, new methods and compositions to treat joint disease and illness are acutely needed to treat these chronic conditions.

In one aspect, the compositions and methods herein described are directed to treat joint disease or illness in a mammal in need thereof. In some embodiments, the joint disease or illness is osteoarthritis. In some embodiments, the joint disease or illness is psoriatic arthritis. In some embodiments, the joint disease or illness is gout.

Among the advantages of the present disclosure over treatments currently available for mammals afflicted with one or more joint disease or illness include the period of relief from symptoms. Upon genetic editing of a cell within a joint, pro-inflammatory signaling is silenced through the targeted gene for the life of that cell and any mitotic progeny. By contrast, biologic treatments require periodic dosing, which may magnify the impact of the host of potentially severe side effects. Among various genetic approaches, the present disclosure is also superior due to, among other reasons, a resistance to leakiness by virtue of modifying a protein receptor, rather than ablating expression of a ligand, which may result in compensatory effects (e.g., buildup of other factors due to lack of negative feedback).

In some embodiments, the present disclosure includes a method for the treatment or prevention of a joint disease or condition in a subject in need thereof, the method comprising administering, to a joint of the subject, a pharmaceutical composition comprising a therapeutically effective amount of a Clustered Regularly Interspaced Short Palindromic Repeats (CRISPR) gene-editing system, the system comprising (i) a CRISPR Associated (Cas) protein; and (ii) at least one guide RNA targeting an IL1R1 gene, IL1RAP gene, TGFBR1 gene, TGFBR2 gene, IL6R gene, IL6ST gene, TNFRSF1A gene, TNFRSF1B gene, TNFRSF3 gene, TNFRSF4 gene, or TNFRSF11A gene or a combination thereof. In some embodiments, the joint disease or condition is osteoarthritis. In some embodiments, the joint disease or condition is psoriatic arthritis. In some embodiments, the joint disease or condition is gout.

In some embodiments, the present disclosure includes a method for the treatment or prevention of an arthritis. Non-limiting examples of arthritis the can be treated using the compositions and methods described herein include post-tramatic arthritis, osteoarthritis (a degenerative condition that affects the joints, most commonly the hips, knees, and hands), rheumatoid arthritis (an autoimmune disorder that causes inflammation in the joints and surrounding tissue), psoriatic arthritis (a type of arthritis that occurs in people with psoriasis, a skin condition characterized by scaly red patches), gout (a type of arthritis caused by the buildup of uric acid crystals in the joints), lupus (a chronic autoimmune disorder that can cause inflammation and damage to the joints, as well as other organs), ankylosing spondylitis (a type of arthritis that primarily affects the spine, causing inflammation and stiffness), reactive arthritis (a type of arthritis that occurs as a reaction to an infection in the body), septic arthritis (a type of arthritis caused by an infection in the joint), juvenile idiopathic arthritis (a form of arthritis that affects children under the age of 16), and fibromyalgia (a chronic pain disorder that can cause widespread pain and stiffness, including in the joints).

In some embodiments, the present disclosure includes a method for the treatment or prevention of pseudogout, Crystal arthropathies (caused by the formation of crystals in the joints, such as gout and pseudogout), or CPPD disease (calcium pyrophosphate deposition disease) also called chondroclacinosis.

In some embodiments, the present disclosure includes a method for the treatment or prevention of rheumatoid arthritis, psoriasis, asthma, inflammatory bowel disease, multiple sclerosis, Alzheimer's disease, Type 2 diabetes, cardiovascular disease, or cancer. In some embodiments, these disorders are treated by administering a CRISPR composition, as described herein, targeting an IL1 receptor, e.g., IL1R1 or IL1RAP.

### B. Osteoarthritis

In one aspect, the present disclosure encompasses treatments for osteoarthritis (OA). In some embodiments, OA treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the OA treatment comprises a CRISPR gene-editing system targeting hIL1R1. In some embodiments, the OA treatment comprises a CRISPR gene-editing system targeting cIL1R1. In some embodiments, the OA treatment comprises a CRISP gene-editing system R targeting eIL1R1. In some embodiments, the OA treatment comprises a CRISPR gene-editing system targeting fIL1R1. In some embodiments, the CRISPR gene-editing system for the treatment of OA comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hIL1R1. In some embodiments, the CRISPR gene-editing system for the treatment of OA comprises one or more sgRNAs targeting exon 2 of hIL1R1. In some embodiments, the CRISPR gene-editing system for the treatment of OA comprises one or more sgRNAs targeting exon 3 of hIL1R1. In some embodiments, the CRISPR gene-editing system for the treatment of OA comprises one or more sgRNAs targeting exon 4 of hIL1R1. In some embodiments, the CRISPR gene-editing system for the treatment of OA comprises one or more sgRNAs targeting exon 5 of hIL1R1. In some embodiments, the CRISPR gene-editing system for the treatment of OA comprises one or more sgRNAs targeting exon 6 of hIL1R1. In some embodiments, the CRISPR gene-editing system for the treatment of OA comprises one or more sgRNAs targeting exon 7 of hIL1R1. In some embodiments, the CRISPR gene-editing system for the treatment of OA comprises one or more sgRNAs targeting exon 8 of hIL1R1. In some embodiments, the CRISPR gene-editing system for the treatment of OA comprises one or more sgRNAs targeting exon 9 of hIL1R1. In some embodiments, the CRISPR gene-editing system for the treatment of OA comprises one or more sgRNAs targeting exon 10 of hIL1R1. In some embodiments, the CRISPR gene-editing system for the treatment of OA comprises one or more sgRNAs targeting exon 11 of hIL1R1. In some embodiments, the CRISPR gene-editing system for the treatment of OA comprises one or more sgRNAs targeting exon 12 of hIL1R1. In some embodiments, the CRISPR gene-editing system for the treatment of OA comprises one or more sgRNAs targeting exon 13 of hIL1R1. In some embodiments, the CRISPR gene-editing system for the treatment of OA comprises one or more sgRNAs targeting exon 14 of hIL1R1. In some embodiments, the CRISPR gene-editing system for the treatment of OA comprises one or more sgRNAs targeting exon 15 of hIL1R1. In some embodiments, the CRISPR gene-editing system for the treatment of OA comprises one or more sgRNAs targeting exon 16 of hIL1R1. In some embodiments, the CRISPR gene-editing system for the treatment of OA comprises one or more sgRNAs targeting exon 17 of hIL1R1. In some embodiments, the CRISPR gene-editing system for the treatment of OA comprises one or more sgRNAs targeting exon 18 of hIL1R1. In some embodiments, the CRISPR gene-editing system for the treatment of OA comprises one or more sgRNAs targeting exon 19 of hIL1R1. In some embodiments, the CRISPR gene-editing system for the treatment of OA comprises one or more sgRNAs targeting exon 20 of hIL1R1. In some embodiments, the CRISPR gene-editing system for the treatment of OA comprises one or more sgRNAs targeting exon 21 of hIL1R1. In some embodiments, the CRISPR gene-editing system for the treatment of OA comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cIL1R1. In some embodiments, the CRISPR gene-editing system for the treatment of OA comprises one or more sgRNAs targeting exon 2 of cIL1R1. In some embodiments, the CRISPR gene-editing system for the treatment of OA comprises one or more sgRNAs targeting exon 3 of cIL1R1. In some embodiments, the CRISPR gene-editing system for the treatment of OA comprises one or more sgRNAs targeting exon 4 of cIL1R1. In some embodiments, the CRISPR gene-editing system for the treatment of OA comprises one or more sgRNAs targeting exon 5 of cIL1R1. In some embodiments, the CRISPR gene-editing system for the treatment of OA comprises one or more sgRNAs targeting exon 6 of cIL1R1. In some embodiments, the CRISPR gene-editing system for the treatment of OA comprises one or more sgRNAs targeting exon 7 of cIL1R1. In some embodiments, the CRISPR gene-editing system for the treatment of OA comprises one or more sgRNAs targeting exon 8 of cIL1R1. In some embodiments, the CRISPR gene-editing system for the treatment of OA comprises one or more sgRNAs targeting exon 9 of cIL1R1. In some embodiments, the CRISPR gene-editing system for the treatment of OA comprises one or more sgRNAs targeting exon 10 of cIL1R1. In some embodiments, the CRISPR gene-editing system for the treatment of OA comprises one or more sgRNAs targeting exon 11 of cIL1R1. In some embodiments, the CRISPR gene-editing system for the treatment of OA comprises one or more sgRNAs targeting exon 12 of cIL1R1. In some embodiments, the CRISPR gene-editing system for the treatment of OA comprises one or more sgRNAs targeting exon 13 of cIL1R1. In some embodiments, the CRISPR gene-editing system for the treatment of OA comprises one or more sgRNAs targeting exon 14 of cIL1R1. In some embodiments, the CRISPR gene-editing system for the treatment of OA comprises one or more sgRNAs targeting exon 15 of cIL1R1. In some embodiments, the CRISPR gene-editing system for the treatment of OA comprises one or more sgRNAs targeting exon 16 of cIL1R1. In some embodiments, the CRISPR gene-editing system for the treatment of OA comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eIL1R1. In some embodiments, the CRISPR gene-editing system for the treatment of OA comprises one or more sgRNAs targeting exon 2 of eIL1R1. In some embodiments, the CRISPR gene-editing system for the treatment of OA comprises one or more sgRNAs targeting exon 3 of eIL1R1. In some embodiments, the CRISPR gene-editing system for the treatment of OA comprises one or more sgRNAs targeting exon 4 of eIL1R1. In some embodiments, the CRISPR gene-editing system for the treatment of OA comprises one or more sgRNAs targeting exon 5 of eIL1R1. In some embodiments, the CRISPR gene-editing system for the treatment of OA comprises one or more sgRNAs targeting exon 6 of eIL1R1. In some embodiments, the CRISPR gene-editing system for the treatment of OA comprises one or more sgRNAs targeting exon 7 of eIL1R1. In some embodiments, the CRISPR gene-editing system for the treatment of OA comprises one or more sgRNAs targeting exon 8 of eIL1R1. In some embodiments, the CRISPR gene-editing system for the treatment of OA comprises one or more sgRNAs targeting exon 9 of eIL1R1. In some embodiments, the CRISPR gene-editing system for the treatment of OA comprises one or more sgRNAs targeting exon 10 of eIL1R1. In some embodiments, the CRISPR gene-editing system for the treatment of OA comprises one or more sgRNAs targeting exon 11 of eIL1R1. In some embodiments, the CRISPR gene-editing system for the treatment of OA comprises one or more sgRNAs targeting exon 12 of eIL1R1. In some embodiments, the CRISPR gene-editing system for the treatment of OA comprises one or more sgRNAs targeting exon 13 of eIL1R1. In some embodiments, the CRISPR gene-editing system for the treatment of OA comprises one or more sgRNAs targeting exon 14 of eIL1R1. In some embodiments, the CRISPR gene-editing system for the treatment of OA comprises one or more sgRNAs targeting exon 15 of eIL1R1. In some embodiments, the CRISPR gene-editing system for the treatment of OA comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fIL1R1. In some embodiments, the CRISPR gene-editing system for the treatment of OA comprises one or more sgRNAs targeting exon 2 of fIL1R1. In some embodiments, the CRISPR gene-editing system for the treatment of OA comprises one or more sgRNAs targeting exon 3 of fIL1R1. In some embodiments, the CRISPR gene-editing system for the treatment of OA comprises one or more sgRNAs targeting exon 4 of fIL1R1. In some embodiments, the CRISPR gene-editing system for the treatment of OA comprises one or more sgRNAs targeting exon 5 of fIL1R1. In some embodiments, the CRISPR gene-editing system for the treatment of OA comprises one or more sgRNAs targeting exon 6 of fIL1R1. In some embodiments, the CRISPR gene-editing system for the treatment of OA comprises one or more sgRNAs targeting exon 7 of fIL1R1. In some embodiments, the CRISPR gene-editing system for the treatment of OA comprises one or more sgRNAs targeting exon 8 of fIL1R1. In some embodiments, the CRISPR gene-editing system for the treatment of OA comprises one or more sgRNAs targeting exon 9 of fIL1R1. In some embodiments, the CRISPR gene-editing system for the treatment of OA comprises one or more sgRNAs targeting exon 10 of fIL1R1. In some embodiments, the CRISPR gene-editing system for the treatment of OA comprises one or more sgRNAs targeting exon 11 of fIL1R. In some embodiments, the CRISPR gene-editing system for the treatment of OA comprises one or more sgRNAs targeting exon 12 of fIL1R1. In some embodiments, the CRISPR gene-editing system for the treatment of OA comprises one or more sgRNAs targeting exon 13 of fIL1R1. In some embodiments, the CRISPR gene-editing system for the treatment of OA comprises one or more sgRNAs targeting exon 14 of fIL1R1. In some embodiments, the CRISPR gene-editing system for the treatment of OA comprises one or more sgRNAs targeting exon 15 of fIL1R1. In some embodiments, the CRISPR gene-editing system for the treatment of OA comprises one or more sgRNAs targeting exon 16 of fIL1R1. In some embodiments, the CRISPR gene-editing system for the treatment of OA comprises one or more sgRNAs targeting exon 17 of fIL1R1. In some embodiments, the CRISPR gene-editing system for the treatment of OA comprises one or more sgRNAs targeting exon 18 of fIL1R1. In some embodiments, the CRISPR gene-editing system for the treatment of OA comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting IL1RAP. In some embodiments, the CRISPR gene-editing system for the treatment of OA comprises one or more sgRNAs targeting hIL1RAP. In some embodiments, the CRISPR gene-editing system for the treatment of OA comprises one or more sgRNAs targeting cIL1RAP. In some embodiments, the CRISPR gene-editing system for the treatment of OA comprises one or more sgRNAs targeting eIL1RAP. In some embodiments, the CRISPR gene-editing system for the treatment of OA comprises one or more sgRNAs targeting fIL1RAP. In some embodiments, the CRISPR gene-editing system for the treatment of OA comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hIL1RAP. In some embodiments, the CRISPR gene-editing system for the treatment of OA comprises one or more sgRNAs targeting exon 2 of hIL1RAP. In some embodiments, the CRISPR gene-editing system for the treatment of OA comprises one or more sgRNAs targeting exon 3 of hIL1RAP. In some embodiments, the CRISPR gene-editing system for the treatment of OA comprises one or more sgRNAs targeting exon 4 of hIL1RAP. In some embodiments, the CRISPR gene-editing system for the treatment of OA comprises one or more sgRNAs targeting exon 5 of hIL1RAP. In some embodiments, the CRISPR gene-editing system for the treatment of OA comprises one or more sgRNAs targeting exon 6 of hIL1RAP. In some embodiments, the CRISPR gene-editing system for the treatment of OA comprises one or more sgRNAs targeting exon 7 of hIL1RAP. In some embodiments, the CRISPR gene-editing system for the treatment of OA comprises one or more sgRNAs targeting exon 8 of hIL1RAP. In some embodiments, the CRISPR gene-editing system for the treatment of OA comprises one or more sgRNAs targeting exon 9 of hIL1RAP. In some embodiments, the CRISPR gene-editing system for the treatment of OA comprises one or more sgRNAs targeting exon 10 of hIL1RAP. In some embodiments, the CRISPR gene-editing system for the treatment of OA comprises one or more sgRNAs targeting exon 11 of hIL1RAP. In some embodiments, the CRISPR gene-editing system for the treatment of OA comprises one or more sgRNAs targeting exon 12 of hIL1RAP. In some embodiments, the CRISPR gene-editing system for the treatment of OA comprises one or more sgRNAs targeting exon 13 of hIL1RAP. In some embodiments, the CRISPR gene-editing system for the treatment of OA comprises one or more sgRNAs targeting exon 14 of hIL1RAP. In some embodiments, the CRISPR gene-editing system for the treatment of OA comprises one or more sgRNAs targeting exon 15 of hIL1RAP. In some embodiments, the CRISPR gene-editing system for the treatment of OA comprises one or more sgRNAs targeting exon 16 of hIL1RAP. In some embodiments, the CRISPR gene-editing system for the treatment of OA comprises one or more sgRNAs targeting exon 17 of hIL1RAP. In some embodiments, the CRISPR gene-editing system for the treatment of OA comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cIL1RAP. In some embodiments, the CRISPR gene-editing system for the treatment of OA comprises one or more sgRNAs targeting exon 2 of cIL1RAP. In some embodiments, the CRISPR gene-editing system for the treatment of OA comprises one or more sgRNAs targeting exon 3 of cIL1RAP. In some embodiments, the CRISPR gene-editing system for the treatment of OA comprises one or more sgRNAs targeting exon 4 of cIL1RAP. In some embodiments, the CRISPR gene-editing system for the treatment of OA comprises one or more sgRNAs targeting exon 5 of cIL1RAP. In some embodiments, the CRISPR gene-editing system for the treatment of OA comprises one or more sgRNAs targeting exon 6 of cIL1RAP. In some embodiments, the CRISPR gene-editing system for the treatment of OA comprises one or more sgRNAs targeting exon 7 of cIL1RAP. In some embodiments, the CRISPR gene-editing system for the treatment of OA comprises one or more sgRNAs targeting exon 8 of cIL1RAP. In some embodiments, the CRISPR gene-editing system for the treatment of OA comprises one or more sgRNAs targeting exon 9 of cIL1RAP. In some embodiments, the CRISPR gene-editing system for the treatment of OA comprises one or more sgRNAs targeting exon 10 of cIL1RAP. In some embodiments, the CRISPR gene-editing system for the treatment of OA comprises one or more sgRNAs targeting exon 11 of cIL1RAP. In some embodiments, the CRISPR gene-editing system for the treatment of OA comprises one or more sgRNAs targeting exon 12 of cIL1RAP. In some embodiments, the CRISPR gene-editing system for the treatment of OA comprises one or more sgRNAs targeting exon 13 of cIL1RAP. In some embodiments, the CRISPR gene-editing system for the treatment of OA comprises one or more sgRNAs targeting exon 14 of cIL1RAP. In some embodiments, the CRISPR gene-editing system for the treatment of OA comprises one or more sgRNAs targeting exon 15 of cIL1RAP. In some embodiments, the CRISPR gene-editing system for the treatment of OA comprises one or more sgRNAs targeting exon 16 of cIL1RAP. In some embodiments, the CRISPR gene-editing system for the treatment of OA comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eIL1RAP. In some embodiments, the CRISPR gene-editing system for the treatment of OA comprises one or more sgRNAs targeting exon 2 of eIL1RAP. In some embodiments, the CRISPR gene-editing system for the treatment of OA comprises one or more sgRNAs targeting exon 3 of eIL1RAP. In some embodiments, the CRISPR gene-editing system for the treatment of OA comprises one or more sgRNAs targeting exon 4 of eIL1RAP. In some embodiments, the CRISPR gene-editing system for the treatment of OA comprises one or more sgRNAs targeting exon 5 of eIL1RAP. In some embodiments, the CRISPR gene-editing system for the treatment of OA comprises one or more sgRNAs targeting exon 6 of eIL1RAP. In some embodiments, the CRISPR gene-editing system for the treatment of OA comprises one or more sgRNAs targeting exon 7 of eIL1RAP. In some embodiments, the CRISPR gene-editing system for the treatment of OA comprises one or more sgRNAs targeting exon 8 of eIL1RAP. In some embodiments, the CRISPR gene-editing system for the treatment of OA comprises one or more sgRNAs targeting exon 9 of eIL1RAP. In some embodiments, the CRISPR gene-editing system for the treatment of OA comprises one or more sgRNAs targeting exon 10 of eIL1RAP. In some embodiments, the CRISPR gene-editing system for the treatment of OA comprises one or more sgRNAs targeting exon 11 of eIL1RAP. In some embodiments, the CRISPR gene-editing system for the treatment of OA comprises one or more sgRNAs targeting exon 12 of eIL1RAP. In some embodiments, the CRISPR gene-editing system for the treatment of OA comprises one or more sgRNAs targeting exon 13 of eIL1RAP. In some embodiments, the CRISPR gene-editing system for the treatment of OA comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fIL1RAP. In some embodiments, the CRISPR gene-editing system for the treatment of OA comprises one or more sgRNAs targeting exon 2 of fIL1RAP. In some embodiments, the CRISPR gene-editing system for the treatment of OA comprises one or more sgRNAs targeting exon 3 of fIL1RAP. In some embodiments, the CRISPR gene-editing system for the treatment of OA comprises one or more sgRNAs targeting exon 4 of fIL1RAP. In some embodiments, the CRISPR gene-editing system for the treatment of OA comprises one or more sgRNAs targeting exon 5 of fIL1RAP. In some embodiments, the CRISPR gene-editing system for the treatment of OA comprises one or more sgRNAs targeting exon 6 of fIL1RAP. In some embodiments, the CRISPR gene-editing system for the treatment of OA comprises one or more sgRNAs targeting exon 7 of fIL1RAP. In some embodiments, the CRISPR gene-editing system for the treatment of OA comprises one or more sgRNAs targeting exon 8 of fIL1RAP. In some embodiments, the CRISPR gene-editing system for the treatment of OA comprises one or more sgRNAs targeting exon 9 of fIL1RAP. In some embodiments, the CRISPR gene-editing system for the treatment of OA comprises one or more sgRNAs targeting exon 10 of fIL1RAP. In some embodiments, the CRISPR gene-editing system for the treatment of OA comprises one or more sgRNAs targeting exon 11 of fIL1RAP. In some embodiments, the CRISPR gene-editing system for the treatment of OA comprises one or more sgRNAs targeting exon 12 of fIL1RAP. In some embodiments, the CRISPR gene-editing system for the treatment of OA comprises one or more sgRNAs targeting exon 13 of fIL1RAP. In some embodiments, the CRISPR gene-editing system for the treatment of OA comprises one or more sgRNAs targeting exon 14 of fIL1RAP. In some embodiments, the CRISPR gene-editing system for the treatment of OA comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TGFBR1. In some embodiments, the CRISPR gene-editing system for the treatment of OA comprises one or more sgRNAs targeting hTGFBR1. In some embodiments, the CRISPR gene-editing system for the treatment of OA comprises one or more sgRNAs targeting cTGFBR1. In some embodiments, the CRISPR gene-editing system for the treatment of OA comprises one or more sgRNAs targeting e TGFBR1. In some embodiments, the CRISPR gene-editing system for the treatment of OA comprises one or more sgRNAs targeting fTGFBR1. In some embodiments, the CRISPR gene-editing system for the treatment of OA comprising one or more sgRNAs targeting an exon of TGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTGFBR1. In some embodiments, the CRISPR gene-editing system for the treatment of OA comprises one or more sgRNAs targeting exon 2 of hTGFBR1. In some embodiments, the CRISPR gene-editing system for the treatment of OA comprises one or more sgRNAs targeting exon 3 of hTGFBR1. In some embodiments, the CRISPR gene-editing system for the treatment of OA comprises one or more sgRNAs targeting exon 4 of hTGFBR1. In some embodiments, the CRISPR gene-editing system for the treatment of OA comprises one or more sgRNAs targeting exon 5 of hTGFBR1. In some embodiments, the CRISPR gene-editing system for the treatment of OA comprises one or more sgRNAs targeting exon 6 of hTGFBR1. In some embodiments, the CRISPR gene-editing system for the treatment of OA comprises one or more sgRNAs targeting exon 7 of hTGFBR1. In some embodiments, the CRISPR gene-editing system for the treatment of OA comprises one or more sgRNAs targeting exon 8 of hTGFBR1. In some embodiments, the CRISPR gene-editing system for the treatment of OA comprises one or more sgRNAs targeting exon 9 of hTGFBR1. In some embodiments, the CRISPR gene-editing system for the treatment of OA comprises one or more sgRNAs targeting exon 10 of hTGFBR1. In some embodiments, the CRISPR gene-editing system for the treatment of OA comprises one or more sgRNAs targeting exon 11 of hTGFBR1. In some embodiments, the CRISPR gene-editing system for the treatment of OA comprising one or more sgRNAs targeting an exon of hTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTGFBR1. In some embodiments, the CRISPR gene-editing system for the treatment of OA comprises one or more sgRNAs targeting exon 2 of cTGFBR1. In some embodiments, the CRISPR gene-editing system for the treatment of OA comprises one or more sgRNAs targeting exon 3 of cTGFBR1. In some embodiments, the CRISPR gene-editing system for the treatment of OA comprises one or more sgRNAs targeting exon 4 of cTGFBR1. In some embodiments, the CRISPR gene-editing system for the treatment of OA comprises one or more sgRNAs targeting exon 5 of cTGFBR1. In some embodiments, the CRISPR gene-editing system for the treatment of OA comprises one or more sgRNAs targeting exon 6 of cTGFBR1. In some embodiments, the CRISPR gene-editing system for the treatment of OA comprises one or more sgRNAs targeting exon 7 of cTGFBR1. In some embodiments, the CRISPR gene-editing system for the treatment of OA comprises one or more sgRNAs targeting exon 8 of cTGFBR1. In some embodiments, the CRISPR gene-editing system for the treatment of OA comprises one or more sgRNAs targeting exon 9 of cTGFBR1. In some embodiments, the CRISPR gene-editing system for the treatment of OA comprising one or more sgRNAs targeting an exon of cTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTGFBR1. In some embodiments, the CRISPR gene-editing system for the treatment of OA comprises one or more sgRNAs targeting exon 2 of eTGFBR1. In some embodiments, the CRISPR gene-editing system for the treatment of OA comprises one or more sgRNAs targeting exon 3 of eTGFBR1. In some embodiments, the CRISPR gene-editing system for the treatment of OA comprises one or more sgRNAs targeting exon 4 of eTGFBR1. In some embodiments, the CRISPR gene-editing system for the treatment of OA comprises one or more sgRNAs targeting exon 5 of eTGFBR1. In some embodiments, the CRISPR gene-editing system for the treatment of OA comprises one or more sgRNAs targeting exon 6 of eTGFBR1. In some embodiments, the CRISPR gene-editing system for the treatment of OA comprises one or more sgRNAs targeting exon 7 of eTGFBR1. In some embodiments, the CRISPR gene-editing system for the treatment of OA comprises one or more sgRNAs targeting exon 8 of eTGFBR1. In some embodiments, the CRISPR gene-editing system for the treatment of OA comprises one or more sgRNAs targeting exon 9 of eTGFBR1. In some embodiments, the CRISPR gene-editing system for the treatment of OA comprises one or more sgRNAs targeting exon 10 of eTGFBR1. In some embodiments, the CRISPR gene-editing system for the treatment of OA comprises one or more sgRNAs targeting exon 11 of eTGFBR1. In some embodiments, the CRISPR gene-editing system for the treatment of OA comprises one or more sgRNAs targeting exon 12 of eTGFBR1. In some embodiments, the CRISPR gene-editing system for the treatment of OA comprising one or more sgRNAs targeting an exon of eTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTGFBR1. In some embodiments, the CRISPR gene-editing system for the treatment of OA comprises one or more sgRNAs targeting exon 2 of fTGFBR1. In some embodiments, the CRISPR gene-editing system for the treatment of OA comprises one or more sgRNAs targeting exon 3 of fTGFBR1. In some embodiments, the CRISPR gene-editing system for the treatment of OA comprises one or more sgRNAs targeting exon 4 of fTGFBR1. In some embodiments, the CRISPR gene-editing system for the treatment of OA comprises one or more sgRNAs targeting exon 5 of fTGFBR1. In some embodiments, the CRISPR gene-editing system for the treatment of OA comprises one or more sgRNAs targeting exon 6 of fTGFBR1. In some embodiments, the CRISPR gene-editing system for the treatment of OA comprises one or more sgRNAs targeting exon 7 of fTGFBR1. In some embodiments, the CRISPR gene-editing system for the treatment of OA comprises one or more sgRNAs targeting exon 8 of fTGFBR1. In some embodiments, the CRISPR gene-editing system for the treatment of OA comprises one or more sgRNAs targeting exon 9 of fTGFBR1. In some embodiments, the CRISPR gene-editing system for the treatment of OA comprises one or more sgRNAs targeting exon 10 of fTGFBR1. In some embodiments, the CRISPR gene-editing system for the treatment of OA comprises one or more sgRNAs targeting exon 11 of fTGFBR1. In some embodiments, the CRISPR gene-editing system for the treatment of OA comprising one or more sgRNAs targeting an exon of fTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TGFBR2. In some embodiments, the CRISPR gene-editing system for the treatment of OA comprises one or more sgRNAs targeting hTGFBR2. In some embodiments, the CRISPR gene-editing system for the treatment of OA comprises one or more sgRNAs targeting cTGFBR2. In some embodiments, the CRISPR gene-editing system for the treatment of OA comprises one or more sgRNAs targeting eTGFBR2. In some embodiments, the CRISPR gene-editing system for the treatment of OA comprises one or more sgRNAs targeting fTGFBR2. In some embodiments, the CRISPR gene-editing system for the treatment of OA comprising one or more sgRNAs targeting an exon of TGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTGFBR2. In some embodiments, the CRISPR gene-editing system for the treatment of OA comprises one or more sgRNAs targeting exon 2 of hTGFBR2. In some embodiments, the CRISPR gene-editing system for the treatment of OA comprises one or more sgRNAs targeting exon 3 of hTGFBR2. In some embodiments, the CRISPR gene-editing system for the treatment of OA comprises one or more sgRNAs targeting exon 4 of hTGFBR2. In some embodiments, the CRISPR gene-editing system for the treatment of OA comprises one or more sgRNAs targeting exon 5 of hTGFBR2. In some embodiments, the CRISPR gene-editing system for the treatment of OA comprises one or more sgRNAs targeting exon 6 of hTGFBR2. In some embodiments, the CRISPR gene-editing system for the treatment of OA comprises one or more sgRNAs targeting exon 7 of hTGFBR2. In some embodiments, the CRISPR gene-editing system for the treatment of OA comprises one or more sgRNAs targeting exon 8 of hTGFBR2. In some embodiments, the CRISPR gene-editing system for the treatment of OA comprises one or more sgRNAs targeting exon 9 of hTGFBR2. In some embodiments, the CRISPR gene-editing system for the treatment of OA comprising one or more sgRNAs targeting an exon of hTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTGFBR2. In some embodiments, the CRISPR gene-editing system for the treatment of OA comprises one or more sgRNAs targeting exon 2 of cTGFBR2. In some embodiments, the CRISPR gene-editing system for the treatment of OA comprises one or more sgRNAs targeting exon 3 of cTGFBR2. In some embodiments, the CRISPR gene-editing system for the treatment of OA comprises one or more sgRNAs targeting exon 4 of cTGFBR2. In some embodiments, the CRISPR gene-editing system for the treatment of OA comprises one or more sgRNAs targeting exon 5 of cTGFBR2. In some embodiments, the CRISPR gene-editing system for the treatment of OA comprises one or more sgRNAs targeting exon 6 of cTGFBR2. In some embodiments, the CRISPR gene-editing system for the treatment of OA comprises one or more sgRNAs targeting exon 7 of cTGFBR2. In some embodiments, the CRISPR gene-editing system for the treatment of OA comprises one or more sgRNAs targeting exon 8 of cTGFBR2. In some embodiments, the CRISPR gene-editing system for the treatment of OA comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTGFBR2. In some embodiments, the CRISPR gene-editing system for the treatment of OA comprises one or more sgRNAs targeting exon 2 of eTGFBR2. In some embodiments, the CRISPR gene-editing system for the treatment of OA comprises one or more sgRNAs targeting exon 3 of eTGFBR2. In some embodiments, the CRISPR gene-editing system for the treatment of OA comprises one or more sgRNAs targeting exon 4 of eTGFBR2. In some embodiments, the CRISPR gene-editing system for the treatment of OA comprises one or more sgRNAs targeting exon 5 of eTGFBR2. In some embodiments, the CRISPR gene-editing system for the treatment of OA comprises one or more sgRNAs targeting exon 6 of eTGFBR2. In some embodiments, the CRISPR gene-editing system for the treatment of OA comprises one or more sgRNAs targeting exon 7 of eTGFBR2. In some embodiments, the CRISPR gene-editing system for the treatment of OA comprises one or more sgRNAs targeting exon 8 of eTGFBR2. In some embodiments, the CRISPR gene-editing system for the treatment of OA comprises one or more sgRNAs targeting exon 9 of eTGFBR2. In some embodiments, the CRISPR gene-editing system for the treatment of OA comprises one or more sgRNAs targeting exon 10 of eTGFBR2. In some embodiments, the CRISPR gene-editing system for the treatment of OA comprising one or more sgRNAs targeting an exon of eTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTGFBR2. In some embodiments, the CRISPR gene-editing system for the treatment of OA comprises one or more sgRNAs targeting exon 2 of fTGFBR2. In some embodiments, the CRISPR gene-editing system for the treatment of OA comprises one or more sgRNAs targeting exon 3 of fTGFBR2. In some embodiments, the CRISPR gene-editing system for the treatment of OA comprises one or more sgRNAs targeting exon 4 of fTGFBR2. In some embodiments, the CRISPR gene-editing system for the treatment of OA comprises one or more sgRNAs targeting exon 5 of fTGFBR2. In some embodiments, the CRISPR gene-editing system for the treatment of OA comprises one or more sgRNAs targeting exon 6 of fTGFBR2. In some embodiments, the CRISPR gene-editing system for the treatment of OA comprises one or more sgRNAs targeting exon 7 of fTGFBR2. In some embodiments, the CRISPR gene-editing system for the treatment of OA comprises one or more sgRNAs targeting exon 8 of fTGFBR2. In some embodiments, the CRISPR gene-editing system for the treatment of OA comprising one or more sgRNAs targeting an exon of fTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting IL6R. In some embodiments, the CRISPR gene-editing system for the treatment of OA comprises one or more sgRNAs targeting hIL6R. In some embodiments, the CRISPR gene-editing system for the treatment of OA comprises one or more sgRNAs targeting cIL6R. In some embodiments, the CRISPR gene-editing system for the treatment of OA comprises one or more sgRNAs targeting eIL6R. In some embodiments, the CRISPR gene-editing system for the treatment of OA comprises one or more sgRNAs targeting fIL6R. In some embodiments, the CRISPR gene-editing system for the treatment of OA comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hIL6R. In some embodiments, the CRISPR gene-editing system for the treatment of OA comprises one or more sgRNAs targeting exon 2 of hIL6R. In some embodiments, the CRISPR gene-editing system for the treatment of OA comprises one or more sgRNAs targeting exon 3 of hIL6R. In some embodiments, the CRISPR gene-editing system for the treatment of OA comprises one or more sgRNAs targeting exon 4 of hIL6R. In some embodiments, the CRISPR gene-editing system for the treatment of OA comprises one or more sgRNAs targeting exon 5 of hIL6R. In some embodiments, the CRISPR gene-editing system for the treatment of OA comprises one or more sgRNAs targeting exon 6 of hIL6R. In some embodiments, the CRISPR gene-editing system for the treatment of OA comprises one or more sgRNAs targeting exon 7 of hIL6R. In some embodiments, the CRISPR gene-editing system for the treatment of OA comprises one or more sgRNAs targeting exon 8 of hIL6R. In some embodiments, the CRISPR gene-editing system for the treatment of OA comprises one or more sgRNAs targeting exon 9 of hIL6R. In some embodiments, the CRISPR gene-editing system for the treatment of OA comprises one or more sgRNAs targeting exon 10 of hIL6R. In some embodiments, the CRISPR gene-editing system for the treatment of OA comprises one or more sgRNAs targeting exon 11 of hIL6R In some embodiments, the CRISPR gene-editing system for the treatment of OA comprises one or more sgRNAs targeting exon 12 of hIL6R. In some embodiments, the CRISPR gene-editing system for the treatment of OA comprises one or more sgRNAs targeting exon 13 of hIL6R. In some embodiments, the CRISPR gene-editing system for the treatment of OA comprises one or more sgRNAs targeting exon 14 of hIL6R. In some embodiments, the CRISPR gene-editing system for the treatment of OA comprises one or more sgRNAs targeting exon 15 of hIL6R. In some embodiments, the CRISPR gene-editing system for the treatment of OA comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cIL6R. In some embodiments, the CRISPR gene-editing system for the treatment of OA comprises one or more sgRNAs targeting exon 2 of cIL6R. In some embodiments, the CRISPR gene-editing system for the treatment of OA comprises one or more sgRNAs targeting exon 3 of cIL6R. In some embodiments, the CRISPR gene-editing system for the treatment of OA comprises one or more sgRNAs targeting exon 4 of cIL6R. In some embodiments, the CRISPR gene-editing system for the treatment of OA comprises one or more sgRNAs targeting exon 5 of cIL6R. In some embodiments, the CRISPR gene-editing system for the treatment of OA comprises one or more sgRNAs targeting exon 6 of cIL6R. In some embodiments, the CRISPR gene-editing system for the treatment of OA comprises one or more sgRNAs targeting exon 7 of cIL6R. In some embodiments, the CRISPR gene-editing system for the treatment of OA comprises one or more sgRNAs targeting exon 8 of cIL6R. In some embodiments, the CRISPR gene-editing system for the treatment of OA comprises one or more sgRNAs targeting exon 9 of cIL6R. In some embodiments, the CRISPR gene-editing system for the treatment of OA comprises one or more sgRNAs targeting exon 10 of cIL6R. In some embodiments, the CRISPR gene-editing system for the treatment of OA comprises one or more sgRNAs targeting exon 11 of cIL6R. In some embodiments, the CRISPR gene-editing system for the treatment of OA comprises one or more sgRNAs targeting exon 12 of cIL6R. In some embodiments, the CRISPR gene-editing system for the treatment of OA comprises one or more sgRNAs targeting exon 13 of cIL6R. In some embodiments, the CRISPR gene-editing system for the treatment of OA comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eIL6R. In some embodiments, the CRISPR gene-editing system for the treatment of OA comprises one or more sgRNAs targeting exon 2 of eIL6R. In some embodiments, the CRISPR gene-editing system for the treatment of OA comprises one or more sgRNAs targeting exon 3 of eIL6R. In some embodiments, the CRISPR gene-editing system for the treatment of OA comprises one or more sgRNAs targeting exon 4 of eIL6R. In some embodiments, the CRISPR gene-editing system for the treatment of OA comprises one or more sgRNAs targeting exon 5 of eIL6R. In some embodiments, the CRISPR gene-editing system for the treatment of OA comprises one or more sgRNAs targeting exon 6 of eIL6R. In some embodiments, the CRISPR gene-editing system for the treatment of OA comprises one or more sgRNAs targeting exon 7 of eIL6R. In some embodiments, the CRISPR gene-editing system for the treatment of OA comprises one or more sgRNAs targeting exon 8 of eIL6R. In some embodiments, the CRISPR gene-editing system for the treatment of OA comprises one or more sgRNAs targeting exon 9 of eIL6R. In some embodiments, the CRISPR gene-editing system for the treatment of OA comprises one or more sgRNAs targeting exon 10 of eIL6R. In some embodiments, the CRISPR gene-editing system for the treatment of OA comprises one or more sgRNAs targeting exon 11 of eIL6R. In some embodiments, the CRISPR gene-editing system for the treatment of OA comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fIL6R. In some embodiments, the CRISPR gene-editing system for the treatment of OA comprises one or more sgRNAs targeting exon 2 of fIL6R. In some embodiments, the CRISPR gene-editing system for the treatment of OA comprises one or more sgRNAs targeting exon 3 of fIL6R. In some embodiments, the CRISPR gene-editing system for the treatment of OA comprises one or more sgRNAs targeting exon 4 of fIL6R. In some embodiments, the CRISPR gene-editing system for the treatment of OA comprises one or more sgRNAs targeting exon 5 of fIL6R. In some embodiments, the CRISPR gene-editing system for the treatment of OA comprises one or more sgRNAs targeting exon 6 of fIL6R. In some embodiments, the CRISPR gene-editing system for the treatment of OA comprises one or more sgRNAs targeting exon 7 of fIL6R. In some embodiments, the CRISPR gene-editing system for the treatment of OA comprises one or more sgRNAs targeting exon 8 of fIL6R. In some embodiments, the CRISPR gene-editing system for the treatment of OA comprises one or more sgRNAs targeting exon 9 of fIL6R. In some embodiments, the CRISPR gene-editing system for the treatment of OA comprises one or more sgRNAs targeting exon 10 of fIL6R. In some embodiments, the CRISPR gene-editing system for the treatment of OA comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting IL6ST. In some embodiments, the CRISPR gene-editing system for the treatment of OA comprises one or more sgRNAs targeting hIL6ST. In some embodiments, the CRISPR gene-editing system for the treatment of OA comprises one or more sgRNAs targeting cIL6ST. In some embodiments, the CRISPR gene-editing system for the treatment of OA comprises one or more sgRNAs targeting eIL6ST. In some embodiments, the CRISPR gene-editing system for the treatment of OA comprises one or more sgRNAs targeting fIL6ST. In some embodiments, the CRISPR gene-editing system for the treatment of OA comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hIL6ST. In some embodiments, the CRISPR gene-editing system for the treatment of OA comprises one or more sgRNAs targeting exon 2 of hIL6ST. In some embodiments, the CRISPR gene-editing system for the treatment of OA comprises one or more sgRNAs targeting exon 3 of hIL6ST. In some embodiments, the CRISPR gene-editing system for the treatment of OA comprises one or more sgRNAs targeting exon 4 of hIL6ST. In some embodiments, the CRISPR gene-editing system for the treatment of OA comprises one or more sgRNAs targeting exon 5 of hIL6ST. In some embodiments, the CRISPR gene-editing system for the treatment of OA comprises one or more sgRNAs targeting exon 6 of hIL6ST. In some embodiments, the CRISPR gene-editing system for the treatment of OA comprises one or more sgRNAs targeting exon 7 of hIL6ST. In some embodiments, the CRISPR gene-editing system for the treatment of OA comprises one or more sgRNAs targeting exon 8 of hIL6ST. In some embodiments, the CRISPR gene-editing system for the treatment of OA comprises one or more sgRNAs targeting exon 9 of hIL6ST. In some embodiments, the CRISPR gene-editing system for the treatment of OA comprises one or more sgRNAs targeting exon 10 of hIL6ST. In some embodiments, the CRISPR gene-editing system for the treatment of OA comprises one or more sgRNAs targeting exon 11 of hIL6ST. In some embodiments, the CRISPR gene-editing system for the treatment of OA comprises one or more sgRNAs targeting exon 12 of hIL6ST. In some embodiments, the CRISPR gene-editing system for the treatment of OA comprises one or more sgRNAs targeting exon 13 of hIL6ST. In some embodiments, the CRISPR gene-editing system for the treatment of OA comprises one or more sgRNAs targeting exon 14 of hIL6ST. In some embodiments, the CRISPR gene-editing system for the treatment of OA comprises one or more sgRNAs targeting exon 15 of hIL6ST. In some embodiments, the CRISPR gene-editing system for the treatment of OA comprises one or more sgRNAs targeting exon 16 of hIL6ST. In some embodiments, the CRISPR gene-editing system for the treatment of OA comprises one or more sgRNAs targeting exon 17 of hIL6ST. In some embodiments, the CRISPR gene-editing system for the treatment of OA comprises one or more sgRNAs targeting exon 18 of hIL6ST. In some embodiments, the CRISPR gene-editing system for the treatment of OA comprises one or more sgRNAs targeting exon 19 of hIL6ST. In some embodiments, the CRISPR gene-editing system for the treatment of OA comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cIL6ST. In some embodiments, the CRISPR gene-editing system for the treatment of OA comprises one or more sgRNAs targeting exon 2 of cIL6ST. In some embodiments, the CRISPR gene-editing system for the treatment of OA comprises one or more sgRNAs targeting exon 3 of cIL6ST. In some embodiments, the CRISPR gene-editing system for the treatment of OA comprises one or more sgRNAs targeting exon 4 of cIL6ST. In some embodiments, the CRISPR gene-editing system for the treatment of OA comprises one or more sgRNAs targeting exon 5 of cIL6ST. In some embodiments, the CRISPR gene-editing system for the treatment of OA comprises one or more sgRNAs targeting exon 6 of cIL6ST. In some embodiments, the CRISPR gene-editing system for the treatment of OA comprises one or more sgRNAs targeting exon 7 of cIL6ST. In some embodiments, the CRISPR gene-editing system for the treatment of OA comprises one or more sgRNAs targeting exon 8 of cIL6ST. In some embodiments, the CRISPR gene-editing system for the treatment of OA comprises one or more sgRNAs targeting exon 9 of cIL6ST. In some embodiments, the CRISPR gene-editing system for the treatment of OA comprises one or more sgRNAs targeting exon 10 of cIL6ST. In some embodiments, the CRISPR gene-editing system for the treatment of OA comprises one or more sgRNAs targeting exon 11 of cIL6ST In some embodiments, the CRISPR gene-editing system for the treatment of OA comprises one or more sgRNAs targeting exon 12 of cIL6ST. In some embodiments, the CRISPR gene-editing system for the treatment of OA comprises one or more sgRNAs targeting exon 13 of cIL6ST. In some embodiments, the CRISPR gene-editing system for the treatment of OA comprises one or more sgRNAs targeting exon 14 of cIL6ST. In some embodiments, the CRISPR gene-editing system for the treatment of OA comprises one or more sgRNAs targeting exon 15 of cIL6ST. In some embodiments, the CRISPR gene-editing system for the treatment of OA comprises one or more sgRNAs targeting exon 16 of cIL6ST. In some embodiments, the CRISPR gene-editing system for the treatment of OA comprises one or more sgRNAs targeting exon 17 of cIL6ST. In some embodiments, the CRISPR gene-editing system for the treatment of OA comprises one or more sgRNAs targeting exon 18 of cIL6ST. In some embodiments, the CRISPR gene-editing system for the treatment of OA comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eIL6ST. In some embodiments, the CRISPR gene-editing system for the treatment of OA comprises one or more sgRNAs targeting exon 2 of eIL6ST. In some embodiments, the CRISPR gene-editing system for the treatment of OA comprises one or more sgRNAs targeting exon 3 of eIL6ST. In some embodiments, the CRISPR gene-editing system for the treatment of OA comprises one or more sgRNAs targeting exon 4 of eIL6ST. In some embodiments, the CRISPR gene-editing system for the treatment of OA comprises one or more sgRNAs targeting exon 5 of eIL6ST. In some embodiments, the CRISPR gene-editing system for the treatment of OA comprises one or more sgRNAs targeting exon 6 of eIL6ST. In some embodiments, the CRISPR gene-editing system for the treatment of OA comprises one or more sgRNAs targeting exon 7 of eIL6ST. In some embodiments, the CRISPR gene-editing system for the treatment of OA comprises one or more sgRNAs targeting exon 8 of eIL6ST. In some embodiments, the CRISPR gene-editing system for the treatment of OA comprises one or more sgRNAs targeting exon 9 of eIL6ST. In some embodiments, the CRISPR gene-editing system for the treatment of OA comprises one or more sgRNAs targeting exon 10 of eIL6ST. In some embodiments, the CRISPR gene-editing system for the treatment of OA comprises one or more sgRNAs targeting exon 11 of eIL6ST In some embodiments, the CRISPR gene-editing system for the treatment of OA comprises one or more sgRNAs targeting exon 12 of eIL6ST. In some embodiments, the CRISPR gene-editing system for the treatment of OA comprises one or more sgRNAs targeting exon 13 of eIL6ST. In some embodiments, the CRISPR gene-editing system for the treatment of OA comprises one or more sgRNAs targeting exon 14 of eIL6ST. In some embodiments, the CRISPR gene-editing system for the treatment of OA comprises one or more sgRNAs targeting exon 15 of eIL6ST. In some embodiments, the CRISPR gene-editing system for the treatment of OA comprises one or more sgRNAs targeting exon 16 of eIL6ST. In some embodiments, the CRISPR gene-editing system for the treatment of OA comprises one or more sgRNAs targeting exon 17 of eIL6ST. In some embodiments, the CRISPR gene-editing system for the treatment of OA comprises one or more sgRNAs targeting exon 18 of eIL6ST. In some embodiments, the CRISPR gene-editing system for the treatment of OA comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fIL6ST. In some embodiments, the CRISPR gene-editing system for the treatment of OA comprises one or more sgRNAs targeting exon 2 of fIL6ST. In some embodiments, the CRISPR gene-editing system for the treatment of OA comprises one or more sgRNAs targeting exon 3 of fIL6ST. In some embodiments, the CRISPR gene-editing system for the treatment of OA comprises one or more sgRNAs targeting exon 4 of fIL6ST. In some embodiments, the CRISPR gene-editing system for the treatment of OA comprises one or more sgRNAs targeting exon 5 of fIL6ST. In some embodiments, the CRISPR gene-editing system for the treatment of OA comprises one or more sgRNAs targeting exon 6 of fIL6ST. In some embodiments, the CRISPR gene-editing system for the treatment of OA comprises one or more sgRNAs targeting exon 7 of fIL6ST. In some embodiments, the CRISPR gene-editing system for the treatment of OA comprises one or more sgRNAs targeting exon 8 of fIL6ST. In some embodiments, the CRISPR gene-editing system for the treatment of OA comprises one or more sgRNAs targeting exon 9 of fIL6ST. In some embodiments, the CRISPR gene-editing system for the treatment of OA comprises one or more sgRNAs targeting exon 10 of fIL6ST. In some embodiments, the CRISPR gene-editing system for the treatment of OA comprises one or more sgRNAs targeting exon 11 of fIL6ST. In some embodiments, the CRISPR gene-editing system for the treatment of OA comprises one or more sgRNAs targeting exon 12 of fIL6ST. In some embodiments, the CRISPR gene-editing system for the treatment of OA comprises one or more sgRNAs targeting exon 13 of fIL6ST. In some embodiments, the CRISPR gene-editing system for the treatment of OA comprises one or more sgRNAs targeting exon 14 of fIL6ST. In some embodiments, the CRISPR gene-editing system for the treatment of OA comprises one or more sgRNAs targeting exon 15 of fIL6ST. In some embodiments, the CRISPR gene-editing system for the treatment of OA comprises one or more sgRNAs targeting exon 16 of fIL6ST. In some embodiments, the CRISPR gene-editing system for the treatment of OA comprises one or more sgRNAs targeting exon 17 of fIL6ST. In some embodiments, the CRISPR gene-editing system for the treatment of OA comprises one or more sgRNAs targeting exon 18 of fIL6ST. In some embodiments, the CRISPR gene-editing system for the treatment of OA comprises one or more sgRNAs targeting exon 19 of fIL6ST. In some embodiments, the CRISPR gene-editing system for the treatment of OA comprises one or more sgRNAs targeting exon 20 of fIL6ST. In some embodiments, the CRISPR gene-editing system for the treatment of OA comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TNFRSF1A. In some embodiments, the CRISPR gene-editing system for the treatment of OA comprises one or more sgRNAs targeting hTNFRSF1A. In some embodiments, the CRISPR gene-editing system for the treatment of OA comprises one or more sgRNAs targeting cTNFRSF1A. In some embodiments, the CRISPR gene-editing system for the treatment of OA comprises one or more sgRNAs targeting eTNFRSF1A. In some embodiments, the CRISPR gene-editing system for the treatment of OA comprises one or more sgRNAs targeting fTNFRSF1A. In some embodiments, the CRISPR gene-editing system for the treatment of OA comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTNFRSF1A. In some embodiments, the CRISPR gene-editing system for the treatment of OA comprises one or more sgRNAs targeting exon 2 of hTNFRSF1A. In some embodiments, the CRISPR gene-editing system for the treatment of OA comprises one or more sgRNAs targeting exon 3 of hTNFRSF1A. In some embodiments, the CRISPR gene-editing system for the treatment of OA comprises one or more sgRNAs targeting exon 4 of hTNFRSF1A. In some embodiments, the CRISPR gene-editing system for the treatment of OA comprises one or more sgRNAs targeting exon 5 of hTNFRSF1A. In some embodiments, the CRISPR gene-editing system for the treatment of OA comprises one or more sgRNAs targeting exon 6 of hTNFRSF1A. In some embodiments, the CRISPR gene-editing system for the treatment of OA comprises one or more sgRNAs targeting exon 7 of hTNFRSF1A. In some embodiments, the CRISPR gene-editing system for the treatment of OA comprises one or more sgRNAs targeting exon 8 of hTNFRSF1A. In some embodiments, the CRISPR gene-editing system for the treatment of OA comprises one or more sgRNAs targeting exon 9 of hTNFRSF1A. In some embodiments, the CRISPR gene-editing system for the treatment of OA comprises one or more sgRNAs targeting exon 10 of hTNFRSF1A. In some embodiments, the CRISPR gene-editing system for the treatment of OA comprises one or more sgRNAs targeting exon 11 of hTNFRSF1A. In some embodiments, the CRISPR gene-editing system for the treatment of OA comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTNFRSF1A. In some embodiments, the CRISPR gene-editing system for the treatment of OA comprises one or more sgRNAs targeting exon 2 of cTNFRSF1A. In some embodiments, the CRISPR gene-editing system for the treatment of OA comprises one or more sgRNAs targeting exon 3 of cTNFRSF1A. In some embodiments, the CRISPR gene-editing system for the treatment of OA comprises one or more sgRNAs targeting exon 4 of cTNFRSF1A. In some embodiments, the CRISPR gene-editing system for the treatment of OA comprises one or more sgRNAs targeting exon 5 of cTNFRSF1A. In some embodiments, the CRISPR gene-editing system for the treatment of OA comprises one or more sgRNAs targeting exon 6 of cTNFRSF1A. In some embodiments, the CRISPR gene-editing system for the treatment of OA comprises one or more sgRNAs targeting exon 7 of cTNFRSF1A. In some embodiments, the CRISPR gene-editing system for the treatment of OA comprises one or more sgRNAs targeting exon 8 of cTNFRSF1A. In some embodiments, the CRISPR gene-editing system for the treatment of OA comprises one or more sgRNAs targeting exon 9 of cTNFRSF1A. In some embodiments, the CRISPR gene-editing system for the treatment of OA comprises one or more sgRNAs targeting exon 10 of cTNFRSF1A. In some embodiments, the CRISPR gene-editing system for the treatment of OA comprises one or more sgRNAs targeting exon 11 of cTNFRSF1A. In some embodiments, the CRISPR gene-editing system for the treatment of OA comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTNFRSF1A. In some embodiments, the CRISPR gene-editing system for the treatment of OA comprises one or more sgRNAs targeting exon 2 of eTNFRSF1A. In some embodiments, the CRISPR gene-editing system for the treatment of OA comprises one or more sgRNAs targeting exon 3 of eTNFRSF1A. In some embodiments, the CRISPR gene-editing system for the treatment of OA comprises one or more sgRNAs targeting exon 4 of eTNFRSF1A. In some embodiments, the CRISPR gene-editing system for the treatment of OA comprises one or more sgRNAs targeting exon 5 of eTNFRSF1A. In some embodiments, the CRISPR gene-editing system for the treatment of OA comprises one or more sgRNAs targeting exon 6 of eTNFRSF1A. In some embodiments, the CRISPR gene-editing system for the treatment of OA comprises one or more sgRNAs targeting exon 7 of eTNFRSF1A. In some embodiments, the CRISPR gene-editing system for the treatment of OA comprises one or more sgRNAs targeting exon 8 of eTNFRSF1A. In some embodiments, the CRISPR gene-editing system for the treatment of OA comprises one or more sgRNAs targeting exon 9 of eTNFRSF1A. In some embodiments, the CRISPR gene-editing system for the treatment of OA comprises one or more sgRNAs targeting exon 10 of eTNFRSF1A. In some embodiments, the CRISPR gene-editing system for the treatment of OA comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTNFRSF1A. In some embodiments, the CRISPR gene-editing system for the treatment of OA comprises one or more sgRNAs targeting exon 2 of fTNFRSF1A. In some embodiments, the CRISPR gene-editing system for the treatment of OA comprises one or more sgRNAs targeting exon 3 of fTNFRSF1A. In some embodiments, the CRISPR gene-editing system for the treatment of OA comprises one or more sgRNAs targeting exon 4 of fTNFRSF1A. In some embodiments, the CRISPR gene-editing system for the treatment of OA comprises one or more sgRNAs targeting exon 5 of fTNFRSF1A. In some embodiments, the CRISPR gene-editing system for the treatment of OA comprises one or more sgRNAs targeting exon 6 of fTNFRSF1A. In some embodiments, the CRISPR gene-editing system for the treatment of OA comprises one or more sgRNAs targeting exon 7 of fTNFRSF1A. In some embodiments, the CRISPR gene-editing system for the treatment of OA comprises one or more sgRNAs targeting exon 8 of fTNFRSF1A. In some embodiments, the CRISPR gene-editing system for the treatment of OA comprises one or more sgRNAs targeting exon 9 of fTNFRSF1A. In some embodiments, the CRISPR gene-editing system for the treatment of OA comprises one or more sgRNAs targeting exon 10 of fTNFRSF1A. In some embodiments, the CRISPR gene-editing system for the treatment of OA comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TNFRSF1B. In some embodiments, the CRISPR gene-editing system for the treatment of OA comprises one or more sgRNAs targeting hTNFRSF1B. In some embodiments, the CRISPR gene-editing system for the treatment of OA comprises one or more sgRNAs targeting cTNFRSF1B. In some embodiments, the CRISPR gene-editing system for the treatment of OA comprises one or more sgRNAs targeting eTNFRSF1B. In some embodiments, the CRISPR gene-editing system for the treatment of OA comprises one or more sgRNAs targeting fTNFRSF1B. In some embodiments, the CRISPR gene-editing system for the treatment of OA comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTNFRSF1B. In some embodiments, the CRISPR gene-editing system for the treatment of OA comprises one or more sgRNAs targeting exon 2 of hTNFRSF1B. In some embodiments, the CRISPR gene-editing system for the treatment of OA comprises one or more sgRNAs targeting exon 3 of hTNFRSF1B. In some embodiments, the CRISPR gene-editing system for the treatment of OA comprises one or more sgRNAs targeting exon 4 of hTNFRSF1B. In some embodiments, the CRISPR gene-editing system for the treatment of OA comprises one or more sgRNAs targeting exon 5 of hTNFRSF1B. In some embodiments, the CRISPR gene-editing system for the treatment of OA comprises one or more sgRNAs targeting exon 6 of hTNFRSF1B. In some embodiments, the CRISPR gene-editing system for the treatment of OA comprises one or more sgRNAs targeting exon 7 of hTNFRSF1B. In some embodiments, the CRISPR gene-editing system for the treatment of OA comprises one or more sgRNAs targeting exon 8 of hTNFRSF1B. In some embodiments, the CRISPR gene-editing system for the treatment of OA comprises one or more sgRNAs targeting exon 9 of hTNFRSF1B. In some embodiments, the CRISPR gene-editing system for the treatment of OA comprises one or more sgRNAs targeting exon 10 of hTNFRSF1B. In some embodiments, the CRISPR gene-editing system for the treatment of OA comprises one or more sgRNAs targeting exon 11 of hTNFRSF1B In some embodiments, the CRISPR gene-editing system for the treatment of OA comprises one or more sgRNAs targeting exon 12 of hTNFRSF1B. In some embodiments, the CRISPR gene-editing system for the treatment of OA comprises one or more sgRNAs targeting exon 13 of hTNFRSF1B. In some embodiments, the CRISPR gene-editing system for the treatment of OA comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTNFRSF1B. In some embodiments, the CRISPR gene-editing system for the treatment of OA comprises one or more sgRNAs targeting exon 2 of cTNFRSF1B. In some embodiments, the CRISPR gene-editing system for the treatment of OA comprises one or more sgRNAs targeting exon 3 of cTNFRSF1B. In some embodiments, the CRISPR gene-editing system for the treatment of OA comprises one or more sgRNAs targeting exon 4 of cTNFRSF1B. In some embodiments, the CRISPR gene-editing system for the treatment of OA comprises one or more sgRNAs targeting exon 5 of cTNFRSF1B. In some embodiments, the CRISPR gene-editing system for the treatment of OA comprises one or more sgRNAs targeting exon 6 of cTNFRSF1B. In some embodiments, the CRISPR gene-editing system for the treatment of OA comprises one or more sgRNAs targeting exon 7 of cTNFRSF1B. In some embodiments, the CRISPR gene-editing system for the treatment of OA comprises one or more sgRNAs targeting exon 8 of cTNFRSF1B. In some embodiments, the CRISPR gene-editing system for the treatment of OA comprises one or more sgRNAs targeting exon 9 of cTNFRSF1B. In some embodiments, the CRISPR gene-editing system for the treatment of OA comprises one or more sgRNAs targeting exon 10 of cTNFRSF1B. In some embodiments, the CRISPR gene-editing system for the treatment of OA comprises one or more sgRNAs targeting exon 11 of cTNFRSF1B. In some embodiments, the CRISPR gene-editing system for the treatment of OA comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTNFRSF1B. In some embodiments, the CRISPR gene-editing system for the treatment of OA comprises one or more sgRNAs targeting exon 2 of eTNFRSF1B. In some embodiments, the CRISPR gene-editing system for the treatment of OA comprises one or more sgRNAs targeting exon 3 of eTNFRSF1B. In some embodiments, the CRISPR gene-editing system for the treatment of OA comprises one or more sgRNAs targeting exon 4 of eTNFRSF1B. In some embodiments, the CRISPR gene-editing system for the treatment of OA comprises one or more sgRNAs targeting exon 5 of eTNFRSF1B. In some embodiments, the CRISPR gene-editing system for the treatment of OA comprises one or more sgRNAs targeting exon 6 of eTNFRSF1B. In some embodiments, the CRISPR gene-editing system for the treatment of OA comprises one or more sgRNAs targeting exon 7 of eTNFRSF1B. In some embodiments, the CRISPR gene-editing system for the treatment of OA comprises one or more sgRNAs targeting exon 8 of eTNFRSF1B. In some embodiments, the CRISPR gene-editing system for the treatment of OA comprises one or more sgRNAs targeting exon 9 of eTNFRSF1B. In some embodiments, the CRISPR gene-editing system for the treatment of OA comprises one or more sgRNAs targeting exon 10 of eTNFRSF1B. In some embodiments, the CRISPR gene-editing system for the treatment of OA comprises one or more sgRNAs targeting exon 11 of eTNFRSF1B. In some embodiments, the CRISPR gene-editing system for the treatment of OA comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTNFRSF1B. In some embodiments, the CRISPR gene-editing system for the treatment of OA comprises one or more sgRNAs targeting exon 2 of fTNFRSF1B. In some embodiments, the CRISPR gene-editing system for the treatment of OA comprises one or more sgRNAs targeting exon 3 of fTNFRSF1B. In some embodiments, the CRISPR gene-editing system for the treatment of OA comprises one or more sgRNAs targeting exon 4 of fTNFRSF1B. In some embodiments, the CRISPR gene-editing system for the treatment of OA comprises one or more sgRNAs targeting exon 5 of fTNFRSF1B. In some embodiments, the CRISPR gene-editing system for the treatment of OA comprises one or more sgRNAs targeting exon 6 of fTNFRSF1B. In some embodiments, the CRISPR gene-editing system for the treatment of OA comprises one or more sgRNAs targeting exon 7 of fTNFRSF1B. In some embodiments, the CRISPR gene-editing system for the treatment of OA comprises one or more sgRNAs targeting exon 8 of fTNFRSF1B. In some embodiments, the CRISPR gene-editing system for the treatment of OA comprises one or more sgRNAs targeting exon 9 of fTNFRSF1B. In some embodiments, the CRISPR gene-editing system for the treatment of OA comprises one or more sgRNAs targeting exon 10 of fTNFRSF1B. In some embodiments, the CRISPR gene-editing system for the treatment of OA comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TNFRSF3. In some embodiments, the CRISPR gene-editing system for the treatment of OA comprises one or more sgRNAs targeting hTNFRSF3. In some embodiments, the CRISPR gene-editing system for the treatment of OA comprises one or more sgRNAs targeting cTNFRSF3. In some embodiments, the CRISPR gene-editing system for the treatment of OA comprises one or more sgRNAs targeting eTNFRSF3. In some embodiments, the CRISPR gene-editing system for the treatment of OA comprises one or more sgRNAs targeting fTNFRSF3. In some embodiments, the CRISPR gene-editing system for the treatment of OA comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTNFRSF3. In some embodiments, the CRISPR gene-editing system for the treatment of OA comprises one or more sgRNAs targeting exon 2 of hTNFRSF3. In some embodiments, the CRISPR gene-editing system for the treatment of OA comprises one or more sgRNAs targeting exon 3 of hTNFRSF3. In some embodiments, the CRISPR gene-editing system for the treatment of OA comprises one or more sgRNAs targeting exon 4 of hTNFRSF3. In some embodiments, the CRISPR gene-editing system for the treatment of OA comprises one or more sgRNAs targeting exon 5 of hTNFRSF3. In some embodiments, the CRISPR gene-editing system for the treatment of OA comprises one or more sgRNAs targeting exon 6 of hTNFRSF3. In some embodiments, the CRISPR gene-editing system for the treatment of OA comprises one or more sgRNAs targeting exon 7 of hTNFRSF3. In some embodiments, the CRISPR gene-editing system for the treatment of OA comprises one or more sgRNAs targeting exon 8 of hTNFRSF3. In some embodiments, the CRISPR gene-editing system for the treatment of OA comprises one or more sgRNAs targeting exon 9 of hTNFRSF3. In some embodiments, the CRISPR gene-editing system for the treatment of OA comprises one or more sgRNAs targeting exon 10 of hTNFRSF3. In some embodiments, the CRISPR gene-editing system for the treatment of OA comprises one or more sgRNAs targeting exon 11 of hTNFRSF3. In some embodiments, the CRISPR gene-editing system for the treatment of OA comprises one or more sgRNAs targeting exon 12 of hTNFRSF3. In some embodiments, the CRISPR gene-editing system for the treatment of OA comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTNFRSF3. In some embodiments, the CRISPR gene-editing system for the treatment of OA comprises one or more sgRNAs targeting exon 2 of cTNFRSF3. In some embodiments, the CRISPR gene-editing system for the treatment of OA comprises one or more sgRNAs targeting exon 3 of cTNFRSF3. In some embodiments, the CRISPR gene-editing system for the treatment of OA comprises one or more sgRNAs targeting exon 4 of cTNFRSF3. In some embodiments, the CRISPR gene-editing system for the treatment of OA comprises one or more sgRNAs targeting exon 5 of cTNFRSF3. In some embodiments, the CRISPR gene-editing system for the treatment of OA comprises one or more sgRNAs targeting exon 6 of cTNFRSF3. In some embodiments, the CRISPR gene-editing system for the treatment of OA comprises one or more sgRNAs targeting exon 7 of cTNFRSF3. In some embodiments, the CRISPR gene-editing system for the treatment of OA comprises one or more sgRNAs targeting exon 8 of cTNFRSF3. In some embodiments, the CRISPR gene-editing system for the treatment of OA comprises one or more sgRNAs targeting exon 9 of cTNFRSF3. In some embodiments, the CRISPR gene-editing system for the treatment of OA comprises one or more sgRNAs targeting exon 10 of cTNFRSF3. In some embodiments, the CRISPR gene-editing system for the treatment of OA comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTNFRSF3. In some embodiments, the CRISPR gene-editing system for the treatment of OA comprises one or more sgRNAs targeting exon 2 of eTNFRSF3. In some embodiments, the CRISPR gene-editing system for the treatment of OA comprises one or more sgRNAs targeting exon 3 of eTNFRSF3. In some embodiments, the CRISPR gene-editing system for the treatment of OA comprises one or more sgRNAs targeting exon 4 of eTNFRSF3. In some embodiments, the CRISPR gene-editing system for the treatment of OA comprises one or more sgRNAs targeting exon 5 of eTNFRSF3. In some embodiments, the CRISPR gene-editing system for the treatment of OA comprises one or more sgRNAs targeting exon 6 of eTNFRSF3. In some embodiments, the CRISPR gene-editing system for the treatment of OA comprises one or more sgRNAs targeting exon 7 of eTNFRSF3. In some embodiments, the CRISPR gene-editing system for the treatment of OA comprises one or more sgRNAs targeting exon 8 of eTNFRSF3. In some embodiments, the CRISPR gene-editing system for the treatment of OA comprises one or more sgRNAs targeting exon 9 of eTNFRSF3. In some embodiments, the CRISPR gene-editing system for the treatment of OA comprises one or more sgRNAs targeting exon 10 of eTNFRSF3. In some embodiments, the CRISPR gene-editing system for the treatment of OA comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTNFRSF3. In some embodiments, the CRISPR gene-editing system for the treatment of OA comprises one or more sgRNAs targeting exon 2 of fTNFRSF3. In some embodiments, the CRISPR gene-editing system for the treatment of OA comprises one or more sgRNAs targeting exon 3 of fTNFRSF3. In some embodiments, the CRISPR gene-editing system for the treatment of OA comprises one or more sgRNAs targeting exon 4 of fTNFRSF3. In some embodiments, the CRISPR gene-editing system for the treatment of OA comprises one or more sgRNAs targeting exon 5 of fTNFRSF3. In some embodiments, the CRISPR gene-editing system for the treatment of OA comprises one or more sgRNAs targeting exon 6 of fTNFRSF3. In some embodiments, the CRISPR gene-editing system for the treatment of OA comprises one or more sgRNAs targeting exon 7 of fTNFRSF3. In some embodiments, the CRISPR gene-editing system for the treatment of OA comprises one or more sgRNAs targeting exon 8 of fTNFRSF3. In some embodiments, the CRISPR gene-editing system for the treatment of OA comprises one or more sgRNAs targeting exon 9 of fTNFRSF3. In some embodiments, the CRISPR gene-editing system for the treatment of OA comprises one or more sgRNAs targeting exon 10 of fTNFRSF3. In some embodiments, the CRISPR gene-editing system for the treatment of OA comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TNFRSF4. In some embodiments, the CRISPR gene-editing system for the treatment of OA comprises one or more sgRNAs targeting hTNFRSF4. In some embodiments, the CRISPR gene-editing system for the treatment of OA comprises one or more sgRNAs targeting cTNFRSF4. In some embodiments, the CRISPR gene-editing system for the treatment of OA comprises one or more sgRNAs targeting eTNFRSF4. In some embodiments, the CRISPR gene-editing system for the treatment of OA comprises one or more sgRNAs targeting fTNFRSF4. In some embodiments, the CRISPR gene-editing system for the treatment of OA comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTNFRSF4. In some embodiments, the CRISPR gene-editing system for the treatment of OA comprises one or more sgRNAs targeting exon 2 of hTNFRSF4. In some embodiments, the CRISPR gene-editing system for the treatment of OA comprises one or more sgRNAs targeting exon 3 of hTNFRSF4. In some embodiments, the CRISPR gene-editing system for the treatment of OA comprises one or more sgRNAs targeting exon 4 of hTNFRSF4. In some embodiments, the CRISPR gene-editing system for the treatment of OA comprises one or more sgRNAs targeting exon 5 of hTNFRSF4. In some embodiments, the CRISPR gene-editing system for the treatment of OA comprises one or more sgRNAs targeting exon 6 of hTNFRSF4. In some embodiments, the CRISPR gene-editing system for the treatment of OA comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTNFRSF4. In some embodiments, the CRISPR gene-editing system for the treatment of OA comprises one or more sgRNAs targeting exon 2 of cTNFRSF4. In some embodiments, the CRISPR gene-editing system for the treatment of OA comprises one or more sgRNAs targeting exon 3 of cTNFRSF4. In some embodiments, the CRISPR gene-editing system for the treatment of OA comprises one or more sgRNAs targeting exon 4 of cTNFRSF4. In some embodiments, the CRISPR gene-editing system for the treatment of OA comprises one or more sgRNAs targeting exon 5 of cTNFRSF4. In some embodiments, the CRISPR gene-editing system for the treatment of OA comprises one or more sgRNAs targeting exon 6 of cTNFRSF4. In some embodiments, the CRISPR gene-editing system for the treatment of OA comprises one or more sgRNAs targeting exon 7 of cTNFRSF4. In some embodiments, the CRISPR gene-editing system for the treatment of OA comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTNFRSF4. In some embodiments, the CRISPR gene-editing system for the treatment of OA comprises one or more sgRNAs targeting exon 2 of eTNFRSF4. In some embodiments, the CRISPR gene-editing system for the treatment of OA comprises one or more sgRNAs targeting exon 3 of eTNFRSF4. In some embodiments, the CRISPR gene-editing system for the treatment of OA comprises one or more sgRNAs targeting exon 4 of eTNFRSF4. In some embodiments, the CRISPR gene-editing system for the treatment of OA comprises one or more sgRNAs targeting exon 5 of eTNFRSF4. In some embodiments, the CRISPR gene-editing system for the treatment of OA comprises one or more sgRNAs targeting exon 6 of eTNFRSF4. In some embodiments, the CRISPR gene-editing system for the treatment of OA comprises one or more sgRNAs targeting exon 7 of eTNFRSF4. In some embodiments, the CRISPR gene-editing system for the treatment of OA comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTNFRSF4. In some embodiments, the CRISPR gene-editing system for the treatment of OA comprises one or more sgRNAs targeting exon 2 of fTNFRSF4. In some embodiments, the CRISPR gene-editing system for the treatment of OA comprises one or more sgRNAs targeting exon 3 of fTNFRSF4. In some embodiments, the CRISPR gene-editing system for the treatment of OA comprises one or more sgRNAs targeting exon 4 of fTNFRSF4. In some embodiments, the CRISPR gene-editing system for the treatment of OA comprises one or more sgRNAs targeting exon 5 of fTNFRSF4. In some embodiments, the CRISPR gene-editing system for the treatment of OA comprises one or more sgRNAs targeting exon 6 of fTNFRSF4. In some embodiments, the CRISPR gene-editing system for the treatment of OA comprises one or more sgRNAs targeting exon 7 of fTNFRSF4. In some embodiments, the CRISPR gene-editing system for the treatment of OA comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting TNFRSF11A. In some embodiments, the CRISPR gene-editing system for the treatment of OA comprises one or more sgRNAs targeting hTNFRSF11A. In some embodiments, the CRISPR gene-editing system for the treatment of OA comprises one or more sgRNAs targeting cTNFRSF11A. In some embodiments, the CRISPR gene-editing system for the treatment of OA comprises one or more sgRNAs targeting eTNFRSF11A. In some embodiments, the CRISPR gene-editing system for the treatment of OA comprises one or more sgRNAs targeting fTNFRSF11A. In some embodiments, the CRISPR gene-editing system for the treatment of OA comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of hTNFRSF11A. In some embodiments, the CRISPR gene-editing system for the treatment of OA comprises one or more sgRNAs targeting exon 2 of hTNFRSF11A. In some embodiments, the CRISPR gene-editing system for the treatment of OA comprises one or more sgRNAs targeting exon 3 of hTNFRSF11A. In some embodiments, the CRISPR gene-editing system for the treatment of OA comprises one or more sgRNAs targeting exon 4 of hTNFRSF11A. In some embodiments, the CRISPR gene-editing system for the treatment of OA comprises one or more sgRNAs targeting exon 5 of hTNFRSF11A. In some embodiments, the CRISPR gene-editing system for the treatment of OA comprises one or more sgRNAs targeting exon 6 of hTNFRSF11A. In some embodiments, the CRISPR gene-editing system for the treatment of OA comprises one or more sgRNAs targeting exon 7 of hTNFRSF11A. In some embodiments, the CRISPR gene-editing system for the treatment of OA comprises one or more sgRNAs targeting exon 8 of hTNFRSF11A. In some embodiments, the CRISPR gene-editing system for the treatment of OA comprises one or more sgRNAs targeting exon 9 of hTNFRSF11A. In some embodiments, the CRISPR gene-editing system for the treatment of OA comprises one or more sgRNAs targeting exon 10 of hTNFRSF11A. In some embodiments, the CRISPR gene-editing system for the treatment of OA comprises one or more sgRNAs targeting exon 11 of hTNFRSF11A. In some embodiments, the CRISPR gene-editing system for the treatment of OA comprises one or more sgRNAs targeting exon 12 of hTNFRSF11A. In some embodiments, the CRISPR gene-editing system for the treatment of OA comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of cTNFRSF11A. In some embodiments, the CRISPR gene-editing system for the treatment of OA comprises one or more sgRNAs targeting exon 2 of cTNFRSF11A. In some embodiments, the CRISPR gene-editing system for the treatment of OA comprises one or more sgRNAs targeting exon 3 of cTNFRSF11A. In some embodiments, the CRISPR gene-editing system for the treatment of OA comprises one or more sgRNAs targeting exon 4 of cTNFRSF11A. In some embodiments, the CRISPR gene-editing system for the treatment of OA comprises one or more sgRNAs targeting exon 5 of cTNFRSF11A. In some embodiments, the CRISPR gene-editing system for the treatment of OA comprises one or more sgRNAs targeting exon 6 of cTNFRSF11A. In some embodiments, the CRISPR gene-editing system for the treatment of OA comprises one or more sgRNAs targeting exon 7 of cTNFRSF11A. In some embodiments, the CRISPR gene-editing system for the treatment of OA comprises one or more sgRNAs targeting exon 8 of cTNFRSF11A. In some embodiments, the CRISPR gene-editing system for the treatment of OA comprises one or more sgRNAs targeting exon 9 of cTNFRSF11A. In some embodiments, the CRISPR gene-editing system for the treatment of OA comprises one or more sgRNAs targeting exon 10 of cTNFRSF11A. In some embodiments, the CRISPR gene-editing system for the treatment of OA comprises one or more sgRNAs targeting exon 11 of cTNFRSF11A. In some embodiments, the CRISPR gene-editing system for the treatment of OA comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of eTNFRSF11A. In some embodiments, the CRISPR gene-editing system for the treatment of OA comprises one or more sgRNAs targeting exon 2 of eTNFRSF11A. In some embodiments, the CRISPR gene-editing system for the treatment of OA comprises one or more sgRNAs targeting exon 3 of eTNFRSF11A. In some embodiments, the CRISPR gene-editing system for the treatment of OA comprises one or more sgRNAs targeting exon 4 of eTNFRSF11A. In some embodiments, the CRISPR gene-editing system for the treatment of OA comprises one or more sgRNAs targeting exon 5 of eTNFRSF11A. In some embodiments, the CRISPR gene-editing system for the treatment of OA comprises one or more sgRNAs targeting exon 6 of eTNFRSF11A. In some embodiments, the CRISPR gene-editing system for the treatment of OA comprises one or more sgRNAs targeting exon 7 of eTNFRSF11A. In some embodiments, the CRISPR gene-editing system for the treatment of OA comprises one or more sgRNAs targeting exon 8 of eTNFRSF11A. In some embodiments, the CRISPR gene-editing system for the treatment of OA comprises one or more sgRNAs targeting exon 9 of eTNFRSF11A. In some embodiments, the CRISPR gene-editing system for the treatment of OA comprises one or more sgRNAs targeting exon 10 of eTNFRSF11A. In some embodiments, the CRISPR gene-editing system for the treatment of OA comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of OA comprises one or more sgRNAs targeting exon 1 of fTNFRSF11A. In some embodiments, the CRISPR gene-editing system for the treatment of OA comprises one or more sgRNAs targeting exon 2 of fTNFRSF11A. In some embodiments, the CRISPR gene-editing system for the treatment of OA comprises one or more sgRNAs targeting exon 3 of fTNFRSF11A. In some embodiments, the CRISPR gene-editing system for the treatment of OA comprises one or more sgRNAs targeting exon 4 of fTNFRSF11A. In some embodiments, the CRISPR gene-editing system for the treatment of OA comprises one or more sgRNAs targeting exon 5 of fTNFRSF11A. In some embodiments, the CRISPR gene-editing system for the treatment of OA comprises one or more sgRNAs targeting exon 6 of fTNFRSF11A. In some embodiments, the CRISPR gene-editing system for the treatment of OA comprises one or more sgRNAs targeting exon 7 of fTNFRSF11A. In some embodiments, the CRISPR gene-editing system for the treatment of OA comprises one or more sgRNAs targeting exon 8 of fTNFRSF11A. In some embodiments, the CRISPR gene-editing system for the treatment of OA comprises one or more sgRNAs targeting exon 9 of fTNFRSF11A. In some embodiments, the CRISPR gene-editing system for the treatment of OA comprises one or more sgRNAs targeting exon 10 of fTNFRSF11A. In some embodiments, the CRISPR gene-editing system for the treatment of OA comprises one or more sgRNAs targeting exon 11 of fTNFRSF11A. In some embodiments, the CRISPR gene-editing system for the treatment of OA comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### C. Psoriatic Arthritis

In one aspect, the present disclosure encompasses treatments for psoriatic arthritis (PsA). In some embodiments, the psoriatic arthritis treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the psoriatic arthritis treatment comprises a CRISPR gene-editing system targeting hIL1R1. In some embodiments, the psoriatic arthritis treatment comprises a CRISPR gene-editing system targeting cIL1R1. In some embodiments, the psoriatic arthritis treatment comprises a CRISP gene-editing system R targeting eIL1R1. In some embodiments, the psoriatic arthritis treatment comprises a CRISPR gene-editing system targeting fIL1R1. In some embodiments, the CRISPR gene-editing system for the treatment of PsA comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hIL1R1. In some embodiments, the CRISPR gene-editing system for the treatment of psoriatic arthritis comprises one or more sgRNAs targeting exon 2 of hIL1R1. In some embodiments, the CRISPR gene-editing system for the treatment of psoriatic arthritis comprises one or more sgRNAs targeting exon 3 of hIL1R1. In some embodiments, the CRISPR gene-editing system for the treatment of psoriatic arthritis comprises one or more sgRNAs targeting exon 4 of hIL1R1. In some embodiments, the CRISPR gene-editing system for the treatment of psoriatic arthritis comprises one or more sgRNAs targeting exon 5 of hIL1R1. In some embodiments, the CRISPR gene-editing system for the treatment of psoriatic arthritis comprises one or more sgRNAs targeting exon 6 of hIL1R1. In some embodiments, the CRISPR gene-editing system for the treatment of psoriatic arthritis comprises one or more sgRNAs targeting exon 7 of hIL1R1. In some embodiments, the CRISPR gene-editing system for the treatment of psoriatic arthritis comprises one or more sgRNAs targeting exon 8 of hIL1R1. In some embodiments, the CRISPR gene-editing system for the treatment of psoriatic arthritis comprises one or more sgRNAs targeting exon 9 of hIL1R1. In some embodiments, the CRISPR gene-editing system for the treatment of psoriatic arthritis comprises one or more sgRNAs targeting exon 10 of hIL1R1. In some embodiments, the CRISPR gene-editing system for the treatment of psoriatic arthritis comprises one or more sgRNAs targeting exon 11 of hIL1R1. In some embodiments, the CRISPR gene-editing system for the treatment of psoriatic arthritis comprises one or more sgRNAs targeting exon 12 of hIL1R1. In some embodiments, the CRISPR gene-editing system for the treatment of psoriatic arthritis comprises one or more sgRNAs targeting exon 13 of hIL1R1. In some embodiments, the CRISPR gene-editing system for the treatment of psoriatic arthritis comprises one or more sgRNAs targeting exon 14 of hIL1R1. In some embodiments, the CRISPR gene-editing system for the treatment of psoriatic arthritis comprises one or more sgRNAs targeting exon 15 of hIL1R1. In some embodiments, the CRISPR gene-editing system for the treatment of psoriatic arthritis comprises one or more sgRNAs targeting exon 16 of hIL1R1. In some embodiments, the CRISPR gene-editing system for the treatment of psoriatic arthritis comprises one or more sgRNAs targeting exon 17 of hIL1R1. In some embodiments, the CRISPR gene-editing system for the treatment of psoriatic arthritis comprises one or more sgRNAs targeting exon 18 of hIL1R1. In some embodiments, the CRISPR gene-editing system for the treatment of psoriatic arthritis comprises one or more sgRNAs targeting exon 19 of hIL1R1. In some embodiments, the CRISPR gene-editing system for the treatment of psoriatic arthritis comprises one or more sgRNAs targeting exon 20 of hIL1R1. In some embodiments, the CRISPR gene-editing system for the treatment of psoriatic arthritis comprises one or more sgRNAs targeting exon 21 of hIL1R1. In some embodiments, the CRISPR gene-editing system for the treatment of PsA comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cIL1R1. In some embodiments, the CRISPR gene-editing system for the treatment of psoriatic arthritis comprises one or more sgRNAs targeting exon 2 of cIL1R. In some embodiments, the CRISPR gene-editing system for the treatment of psoriatic arthritis comprises one or more sgRNAs targeting exon 3 of cIL1R1. In some embodiments, the CRISPR gene-editing system for the treatment of psoriatic arthritis comprises one or more sgRNAs targeting exon 4 of cIL1R1. In some embodiments, the CRISPR gene-editing system for the treatment of psoriatic arthritis comprises one or more sgRNAs targeting exon 5 of cIL1R1. In some embodiments, the CRISPR gene-editing system for the treatment of psoriatic arthritis comprises one or more sgRNAs targeting exon 6 of cIL1R1. In some embodiments, the CRISPR gene-editing system for the treatment of psoriatic arthritis comprises one or more sgRNAs targeting exon 7 of cIL1R1. In some embodiments, the CRISPR gene-editing system for the treatment of psoriatic arthritis comprises one or more sgRNAs targeting exon 8 of cIL1R1. In some embodiments, the CRISPR gene-editing system for the treatment of psoriatic arthritis comprises one or more sgRNAs targeting exon 9 of cIL1R1. In some embodiments, the CRISPR gene-editing system for the treatment of psoriatic arthritis comprises one or more sgRNAs targeting exon 10 of cIL1R1. In some embodiments, the CRISPR gene-editing system for the treatment of psoriatic arthritis comprises one or more sgRNAs targeting exon 11 of cIL1R1. In some embodiments, the CRISPR gene-editing system for the treatment of psoriatic arthritis comprises one or more sgRNAs targeting exon 12 of cIL1R1. In some embodiments, the CRISPR gene-editing system for the treatment of psoriatic arthritis comprises one or more sgRNAs targeting exon 13 of cIL1R1. In some embodiments, the CRISPR gene-editing system for the treatment of psoriatic arthritis comprises one or more sgRNAs targeting exon 14 of cIL1R1. In some embodiments, the CRISPR gene-editing system for the treatment of psoriatic arthritis comprises one or more sgRNAs targeting exon 15 of cIL1R1. In some embodiments, the CRISPR gene-editing system for the treatment of psoriatic arthritis comprises one or more sgRNAs targeting exon 16 of cIL1R1. In some embodiments, the CRISPR gene-editing system for the treatment of PsA comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eIL1R1. In some embodiments, the CRISPR gene-editing system for the treatment of psoriatic arthritis comprises one or more sgRNAs targeting exon 2 of eIL1R1. In some embodiments, the CRISPR gene-editing system for the treatment of psoriatic arthritis comprises one or more sgRNAs targeting exon 3 of eIL1R1. In some embodiments, the CRISPR gene-editing system for the treatment of psoriatic arthritis comprises one or more sgRNAs targeting exon 4 of eIL1R1. In some embodiments, the CRISPR gene-editing system for the treatment of psoriatic arthritis comprises one or more sgRNAs targeting exon 5 of eIL1R1. In some embodiments, the CRISPR gene-editing system for the treatment of psoriatic arthritis comprises one or more sgRNAs targeting exon 6 of eIL1R1. In some embodiments, the CRISPR gene-editing system for the treatment of psoriatic arthritis comprises one or more sgRNAs targeting exon 7 of eIL1R1. In some embodiments, the CRISPR gene-editing system for the treatment of psoriatic arthritis comprises one or more sgRNAs targeting exon 8 of eIL1R1. In some embodiments, the CRISPR gene-editing system for the treatment of psoriatic arthritis comprises one or more sgRNAs targeting exon 9 of eIL1R1. In some embodiments, the CRISPR gene-editing system for the treatment of psoriatic arthritis comprises one or more sgRNAs targeting exon 10 of eIL1R1. In some embodiments, the CRISPR gene-editing system for the treatment of psoriatic arthritis comprises one or more sgRNAs targeting exon 11 of eIL1R1. In some embodiments, the CRISPR gene-editing system for the treatment of psoriatic arthritis comprises one or more sgRNAs targeting exon 12 of eIL1R1. In some embodiments, the CRISPR gene-editing system for the treatment of psoriatic arthritis comprises one or more sgRNAs targeting exon 13 of eIL1R1. In some embodiments, the CRISPR gene-editing system for the treatment of psoriatic arthritis comprises one or more sgRNAs targeting exon 14 of eIL1R1. In some embodiments, the CRISPR gene-editing system for the treatment of psoriatic arthritis comprises one or more sgRNAs targeting exon 15 of eIL1R1. In some embodiments, the CRISPR gene-editing system for the treatment of PsA comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fIL1R1. In some embodiments, the CRISPR gene-editing system for the treatment of psoriatic arthritis comprises one or more sgRNAs targeting exon 2 of fIL1R1. In some embodiments, the CRISPR gene-editing system for the treatment of psoriatic arthritis comprises one or more sgRNAs targeting exon 3 of fIL1R1. In some embodiments, the CRISPR gene-editing system for the treatment of psoriatic arthritis comprises one or more sgRNAs targeting exon 4 of fIL1R1. In some embodiments, the CRISPR gene-editing system for the treatment of psoriatic arthritis comprises one or more sgRNAs targeting exon 5 of fIL1R1. In some embodiments, the CRISPR gene-editing system for the treatment of psoriatic arthritis comprises one or more sgRNAs targeting exon 6 of fIL1R1. In some embodiments, the CRISPR gene-editing system for the treatment of psoriatic arthritis comprises one or more sgRNAs targeting exon 7 of fIL1R1. In some embodiments, the CRISPR gene-editing system for the treatment of psoriatic arthritis comprises one or more sgRNAs targeting exon 8 of fIL1R1. In some embodiments, the CRISPR gene-editing system for the treatment of psoriatic arthritis comprises one or more sgRNAs targeting exon 9 of fIL1R1. In some embodiments, the CRISPR gene-editing system for the treatment of psoriatic arthritis comprises one or more sgRNAs targeting exon 10 of fIL1R1. In some embodiments, the CRISPR gene-editing system for the treatment of psoriatic arthritis comprises one or more sgRNAs targeting exon 11 of fIL1R1. In some embodiments, the CRISPR gene-editing system for the treatment of psoriatic arthritis comprises one or more sgRNAs targeting exon 12 of fIL1R1. In some embodiments, the CRISPR gene-editing system for the treatment of psoriatic arthritis comprises one or more sgRNAs targeting exon 13 of fIL1R1. In some embodiments, the CRISPR gene-editing system for the treatment of psoriatic arthritis comprises one or more sgRNAs targeting exon 14 of fIL1R1. In some embodiments, the CRISPR gene-editing system for the treatment of psoriatic arthritis comprises one or more sgRNAs targeting exon 15 of fIL1R1. In some embodiments, the CRISPR gene-editing system for the treatment of psoriatic arthritis comprises one or more sgRNAs targeting exon 16 of fIL1R1. In some embodiments, the CRISPR gene-editing system for the treatment of psoriatic arthritis comprises one or more sgRNAs targeting exon 17 of fIL1R. In some embodiments, the CRISPR gene-editing system for the treatment of psoriatic arthritis comprises one or more sgRNAs targeting exon 18 of fIL1R1. In some embodiments, the CRISPR gene-editing system for the treatment of PsA comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting IL1RAP. In some embodiments, the CRISPR gene-editing system for the treatment of psoriatic arthritis comprises one or more sgRNAs targeting hIL1RAP. In some embodiments, the CRISPR gene-editing system for the treatment of psoriatic arthritis comprises one or more sgRNAs targeting cIL1RAP. In some embodiments, the CRISPR gene-editing system for the treatment of psoriatic arthritis comprises one or more sgRNAs targeting eIL1RAP. In some embodiments, the CRISPR gene-editing system for the treatment of psoriatic arthritis comprises one or more sgRNAs targeting fIL1RAP. In some embodiments, the CRISPR gene-editing system for the treatment of PsA comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hIL1RAP. In some embodiments, the CRISPR gene-editing system for the treatment of psoriatic arthritis comprises one or more sgRNAs targeting exon 2 of hIL1RAP. In some embodiments, the CRISPR gene-editing system for the treatment of psoriatic arthritis comprises one or more sgRNAs targeting exon 3 of hIL1RAP. In some embodiments, the CRISPR gene-editing system for the treatment of psoriatic arthritis comprises one or more sgRNAs targeting exon 4 of hIL1RAP. In some embodiments, the CRISPR gene-editing system for the treatment of psoriatic arthritis comprises one or more sgRNAs targeting exon 5 of hIL1RAP. In some embodiments, the CRISPR gene-editing system for the treatment of psoriatic arthritis comprises one or more sgRNAs targeting exon 6 of hIL1RAP. In some embodiments, the CRISPR gene-editing system for the treatment of psoriatic arthritis comprises one or more sgRNAs targeting exon 7 of hIL1RAP. In some embodiments, the CRISPR gene-editing system for the treatment of psoriatic arthritis comprises one or more sgRNAs targeting exon 8 of hIL1RAP. In some embodiments, the CRISPR gene-editing system for the treatment of psoriatic arthritis comprises one or more sgRNAs targeting exon 9 of hIL1RAP. In some embodiments, the CRISPR gene-editing system for the treatment of psoriatic arthritis comprises one or more sgRNAs targeting exon 10 of hIL1RAP. In some embodiments, the CRISPR gene-editing system for the treatment of psoriatic arthritis comprises one or more sgRNAs targeting exon 11 of hIL1RAP. In some embodiments, the CRISPR gene-editing system for the treatment of psoriatic arthritis comprises one or more sgRNAs targeting exon 12 of hIL1RAP. In some embodiments, the CRISPR gene-editing system for the treatment of psoriatic arthritis comprises one or more sgRNAs targeting exon 13 of hIL1RAP. In some embodiments, the CRISPR gene-editing system for the treatment of psoriatic arthritis comprises one or more sgRNAs targeting exon 14 of hIL1RAP. In some embodiments, the CRISPR gene-editing system for the treatment of psoriatic arthritis comprises one or more sgRNAs targeting exon 15 of hIL1RAP. In some embodiments, the CRISPR gene-editing system for the treatment of psoriatic arthritis comprises one or more sgRNAs targeting exon 16 of hIL1RAP. In some embodiments, the CRISPR gene-editing system for the treatment of psoriatic arthritis comprises one or more sgRNAs targeting exon 17 of hIL1RAP. In some embodiments, the CRISPR gene-editing system for the treatment of PsA comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cIL1RAP. In some embodiments, the CRISPR gene-editing system for the treatment of psoriatic arthritis comprises one or more sgRNAs targeting exon 2 of cIL1RAP. In some embodiments, the CRISPR gene-editing system for the treatment of psoriatic arthritis comprises one or more sgRNAs targeting exon 3 of cIL1RAP. In some embodiments, the CRISPR gene-editing system for the treatment of psoriatic arthritis comprises one or more sgRNAs targeting exon 4 of cIL1RAP. In some embodiments, the CRISPR gene-editing system for the treatment of psoriatic arthritis comprises one or more sgRNAs targeting exon 5 of cIL1RAP. In some embodiments, the CRISPR gene-editing system for the treatment of psoriatic arthritis comprises one or more sgRNAs targeting exon 6 of cIL1RAP. In some embodiments, the CRISPR gene-editing system for the treatment of psoriatic arthritis comprises one or more sgRNAs targeting exon 7 of cIL1RAP. In some embodiments, the CRISPR gene-editing system for the treatment of psoriatic arthritis comprises one or more sgRNAs targeting exon 8 of cIL1RAP. In some embodiments, the CRISPR gene-editing system for the treatment of psoriatic arthritis comprises one or more sgRNAs targeting exon 9 of cIL1RAP. In some embodiments, the CRISPR gene-editing system for the treatment of psoriatic arthritis comprises one or more sgRNAs targeting exon 10 of cIL1RAP. In some embodiments, the CRISPR gene-editing system for the treatment of psoriatic arthritis comprises one or more sgRNAs targeting exon 11 of cIL1RAP. In some embodiments, the CRISPR gene-editing system for the treatment of psoriatic arthritis comprises one or more sgRNAs targeting exon 12 of cIL1RAP. In some embodiments, the CRISPR gene-editing system for the treatment of psoriatic arthritis comprises one or more sgRNAs targeting exon 13 of cIL1RAP. In some embodiments, the CRISPR gene-editing system for the treatment of psoriatic arthritis comprises one or more sgRNAs targeting exon 14 of cIL1RAP. In some embodiments, the CRISPR gene-editing system for the treatment of psoriatic arthritis comprises one or more sgRNAs targeting exon 15 of cIL1RAP. In some embodiments, the CRISPR gene-editing system for the treatment of psoriatic arthritis comprises one or more sgRNAs targeting exon 16 of cIL1RAP. In some embodiments, the CRISPR gene-editing system for the treatment of PsA comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eIL1RAP. In some embodiments, the CRISPR gene-editing system for the treatment of psoriatic arthritis comprises one or more sgRNAs targeting exon 2 of eIL1RAP. In some embodiments, the CRISPR gene-editing system for the treatment of psoriatic arthritis comprises one or more sgRNAs targeting exon 3 of eIL1RAP. In some embodiments, the CRISPR gene-editing system for the treatment of psoriatic arthritis comprises one or more sgRNAs targeting exon 4 of eIL1RAP. In some embodiments, the CRISPR gene-editing system for the treatment of psoriatic arthritis comprises one or more sgRNAs targeting exon 5 of eIL1RAP. In some embodiments, the CRISPR gene-editing system for the treatment of psoriatic arthritis comprises one or more sgRNAs targeting exon 6 of eIL1RAP. In some embodiments, the CRISPR gene-editing system for the treatment of psoriatic arthritis comprises one or more sgRNAs targeting exon 7 of eIL1RAP. In some embodiments, the CRISPR gene-editing system for the treatment of psoriatic arthritis comprises one or more sgRNAs targeting exon 8 of eIL1RAP. In some embodiments, the CRISPR gene-editing system for the treatment of psoriatic arthritis comprises one or more sgRNAs targeting exon 9 of eIL1RAP. In some embodiments, the CRISPR gene-editing system for the treatment of psoriatic arthritis comprises one or more sgRNAs targeting exon 10 of eIL1RAP. In some embodiments, the CRISPR gene-editing system for the treatment of psoriatic arthritis comprises one or more sgRNAs targeting exon 11 of eIL1RAP. In some embodiments, the CRISPR gene-editing system for the treatment of psoriatic arthritis comprises one or more sgRNAs targeting exon 12 of eIL1RAP. In some embodiments, the CRISPR gene-editing system for the treatment of psoriatic arthritis comprises one or more sgRNAs targeting exon 13 of eIL1RAP. In some embodiments, the CRISPR gene-editing system for the treatment of PsA comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fIL1RAP. In some embodiments, the CRISPR gene-editing system for the treatment of psoriatic arthritis comprises one or more sgRNAs targeting exon 2 of fIL1RAP. In some embodiments, the CRISPR gene-editing system for the treatment of psoriatic arthritis comprises one or more sgRNAs targeting exon 3 of fIL1RAP. In some embodiments, the CRISPR gene-editing system for the treatment of psoriatic arthritis comprises one or more sgRNAs targeting exon 4 of fIL1RAP. In some embodiments, the CRISPR gene-editing system for the treatment of psoriatic arthritis comprises one or more sgRNAs targeting exon 5 of fIL1RAP. In some embodiments, the CRISPR gene-editing system for the treatment of psoriatic arthritis comprises one or more sgRNAs targeting exon 6 of fIL1RAP. In some embodiments, the CRISPR gene-editing system for the treatment of psoriatic arthritis comprises one or more sgRNAs targeting exon 7 of fIL1RAP. In some embodiments, the CRISPR gene-editing system for the treatment of psoriatic arthritis comprises one or more sgRNAs targeting exon 8 of fIL1RAP. In some embodiments, the CRISPR gene-editing system for the treatment of psoriatic arthritis comprises one or more sgRNAs targeting exon 9 of fIL1RAP. In some embodiments, the CRISPR gene-editing system for the treatment of psoriatic arthritis comprises one or more sgRNAs targeting exon 10 of fIL1RAP. In some embodiments, the CRISPR gene-editing system for the treatment of psoriatic arthritis comprises one or more sgRNAs targeting exon 11 of fIL1RAP. In some embodiments, the CRISPR gene-editing system for the treatment of psoriatic arthritis comprises one or more sgRNAs targeting exon 12 of fIL1RAP. In some embodiments, the CRISPR gene-editing system for the treatment of psoriatic arthritis comprises one or more sgRNAs targeting exon 13 of fIL1RAP. In some embodiments, the CRISPR gene-editing system for the treatment of psoriatic arthritis comprises one or more sgRNAs targeting exon 14 of fIL1RAP. In some embodiments, the CRISPR gene-editing system for the treatment of PsA comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TGFBR1. In some embodiments, the CRISPR gene-editing system for the treatment of psoriatic arthritis comprises one or more sgRNAs targeting hTGFBR1. In some embodiments, the CRISPR gene-editing system for the treatment of psoriatic arthritis comprises one or more sgRNAs targeting cTGFBR1. In some embodiments, the CRISPR gene-editing system for the treatment of psoriatic arthritis comprises one or more sgRNAs targeting e TGFBR1. In some embodiments, the CRISPR gene-editing system for the treatment of psoriatic arthritis comprises one or more sgRNAs targeting fTGFBR1. In some embodiments, the CRISPR gene-editing system for the treatment of PsA comprising one or more sgRNAs targeting an exon of TGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTGFBR1. In some embodiments, the CRISPR gene-editing system for the treatment of psoriatic arthritis comprises one or more sgRNAs targeting exon 2 of hTGFBR1. In some embodiments, the CRISPR gene-editing system for the treatment of psoriatic arthritis comprises one or more sgRNAs targeting exon 3 of hTGFBR1. In some embodiments, the CRISPR gene-editing system for the treatment of psoriatic arthritis comprises one or more sgRNAs targeting exon 4 of hTGFBR1. In some embodiments, the CRISPR gene-editing system for the treatment of psoriatic arthritis comprises one or more sgRNAs targeting exon 5 of hTGFBR1. In some embodiments, the CRISPR gene-editing system for the treatment of psoriatic arthritis comprises one or more sgRNAs targeting exon 6 of hTGFBR1. In some embodiments, the CRISPR gene-editing system for the treatment of psoriatic arthritis comprises one or more sgRNAs targeting exon 7 of hTGFBR1. In some embodiments, the CRISPR gene-editing system for the treatment of psoriatic arthritis comprises one or more sgRNAs targeting exon 8 of hTGFBR1. In some embodiments, the CRISPR gene-editing system for the treatment of psoriatic arthritis comprises one or more sgRNAs targeting exon 9 of hTGFBR1. In some embodiments, the CRISPR gene-editing system for the treatment of psoriatic arthritis comprises one or more sgRNAs targeting exon 10 of hTGFBR1. In some embodiments, the CRISPR gene-editing system for the treatment of psoriatic arthritis comprises one or more sgRNAs targeting exon 11 of hTGFBR1. In some embodiments, the CRISPR gene-editing system for the treatment of PsA comprising one or more sgRNAs targeting an exon of hTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTGFBR1. In some embodiments, the CRISPR gene-editing system for the treatment of psoriatic arthritis comprises one or more sgRNAs targeting exon 2 of cTGFBR1. In some embodiments, the CRISPR gene-editing system for the treatment of psoriatic arthritis comprises one or more sgRNAs targeting exon 3 of cTGFBR1. In some embodiments, the CRISPR gene-editing system for the treatment of psoriatic arthritis comprises one or more sgRNAs targeting exon 4 of cTGFBR1. In some embodiments, the CRISPR gene-editing system for the treatment of psoriatic arthritis comprises one or more sgRNAs targeting exon 5 of cTGFBR1. In some embodiments, the CRISPR gene-editing system for the treatment of psoriatic arthritis comprises one or more sgRNAs targeting exon 6 of cTGFBR1. In some embodiments, the CRISPR gene-editing system for the treatment of psoriatic arthritis comprises one or more sgRNAs targeting exon 7 of cTGFBR1. In some embodiments, the CRISPR gene-editing system for the treatment of psoriatic arthritis comprises one or more sgRNAs targeting exon 8 of cTGFBR1. In some embodiments, the CRISPR gene-editing system for the treatment of psoriatic arthritis comprises one or more sgRNAs targeting exon 9 of cTGFBR1. In some embodiments, the CRISPR gene-editing system for the treatment of PsA comprising one or more sgRNAs targeting an exon of cTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTGFBR1. In some embodiments, the CRISPR gene-editing system for the treatment of psoriatic arthritis comprises one or more sgRNAs targeting exon 2 of eTGFBR1. In some embodiments, the CRISPR gene-editing system for the treatment of psoriatic arthritis comprises one or more sgRNAs targeting exon 3 of eTGFBR1. In some embodiments, the CRISPR gene-editing system for the treatment of psoriatic arthritis comprises one or more sgRNAs targeting exon 4 of eTGFBR1. In some embodiments, the CRISPR gene-editing system for the treatment of psoriatic arthritis comprises one or more sgRNAs targeting exon 5 of eTGFBR1. In some embodiments, the CRISPR gene-editing system for the treatment of psoriatic arthritis comprises one or more sgRNAs targeting exon 6 of eTGFBR1. In some embodiments, the CRISPR gene-editing system for the treatment of psoriatic arthritis comprises one or more sgRNAs targeting exon 7 of eTGFBR1. In some embodiments, the CRISPR gene-editing system for the treatment of psoriatic arthritis comprises one or more sgRNAs targeting exon 8 of eTGFBR1. In some embodiments, the CRISPR gene-editing system for the treatment of psoriatic arthritis comprises one or more sgRNAs targeting exon 9 of eTGFBR1. In some embodiments, the CRISPR gene-editing system for the treatment of psoriatic arthritis comprises one or more sgRNAs targeting exon 10 of eTGFBR1. In some embodiments, the CRISPR gene-editing system for the treatment of psoriatic arthritis comprises one or more sgRNAs targeting exon 11 of eTGFBR1. In some embodiments, the CRISPR gene-editing system for the treatment of psoriatic arthritis comprises one or more sgRNAs targeting exon 12 of eTGFBR1. In some embodiments, the CRISPR gene-editing system for the treatment of PsA comprising one or more sgRNAs targeting an exon of eTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTGFBR1. In some embodiments, the CRISPR gene-editing system for the treatment of psoriatic arthritis comprises one or more sgRNAs targeting exon 2 of fTGFBR1. In some embodiments, the CRISPR gene-editing system for the treatment of psoriatic arthritis comprises one or more sgRNAs targeting exon 3 of fTGFBR1. In some embodiments, the CRISPR gene-editing system for the treatment of psoriatic arthritis comprises one or more sgRNAs targeting exon 4 of fTGFBR1. In some embodiments, the CRISPR gene-editing system for the treatment of psoriatic arthritis comprises one or more sgRNAs targeting exon 5 of fTGFBR1. In some embodiments, the CRISPR gene-editing system for the treatment of psoriatic arthritis comprises one or more sgRNAs targeting exon 6 of fTGFBR1. In some embodiments, the CRISPR gene-editing system for the treatment of psoriatic arthritis comprises one or more sgRNAs targeting exon 7 of fTGFBR1. In some embodiments, the CRISPR gene-editing system for the treatment of psoriatic arthritis comprises one or more sgRNAs targeting exon 8 of fTGFBR1. In some embodiments, the CRISPR gene-editing system for the treatment of psoriatic arthritis comprises one or more sgRNAs targeting exon 9 of fTGFBR1. In some embodiments, the CRISPR gene-editing system for the treatment of psoriatic arthritis comprises one or more sgRNAs targeting exon 10 of fTGFBR1. In some embodiments, the CRISPR gene-editing system for the treatment of psoriatic arthritis comprises one or more sgRNAs targeting exon 11 of fTGFBR1. In some embodiments, the CRISPR gene-editing system for the treatment of PsA comprising one or more sgRNAs targeting an exon of fTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TGFBR2. In some embodiments, the CRISPR gene-editing system for the treatment of psoriatic arthritis comprises one or more sgRNAs targeting hTGFBR2. In some embodiments, the CRISPR gene-editing system for the treatment of psoriatic arthritis comprises one or more sgRNAs targeting cTGFBR2. In some embodiments, the CRISPR gene-editing system for the treatment of psoriatic arthritis comprises one or more sgRNAs targeting eTGFBR2. In some embodiments, the CRISPR gene-editing system for the treatment of psoriatic arthritis comprises one or more sgRNAs targeting fTGFBR2. In some embodiments, the CRISPR gene-editing system for the treatment of PsA comprising one or more sgRNAs targeting an exon of TGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTGFBR2. In some embodiments, the CRISPR gene-editing system for the treatment of psoriatic arthritis comprises one or more sgRNAs targeting exon 2 of hTGFBR2. In some embodiments, the CRISPR gene-editing system for the treatment of psoriatic arthritis comprises one or more sgRNAs targeting exon 3 of hTGFBR2. In some embodiments, the CRISPR gene-editing system for the treatment of psoriatic arthritis comprises one or more sgRNAs targeting exon 4 of hTGFBR2. In some embodiments, the CRISPR gene-editing system for the treatment of psoriatic arthritis comprises one or more sgRNAs targeting exon 5 of hTGFBR2. In some embodiments, the CRISPR gene-editing system for the treatment of psoriatic arthritis comprises one or more sgRNAs targeting exon 6 of hTGFBR2. In some embodiments, the CRISPR gene-editing system for the treatment of psoriatic arthritis comprises one or more sgRNAs targeting exon 7 of hTGFBR2. In some embodiments, the CRISPR gene-editing system for the treatment of psoriatic arthritis comprises one or more sgRNAs targeting exon 8 of hTGFBR2. In some embodiments, the CRISPR gene-editing system for the treatment of psoriatic arthritis comprises one or more sgRNAs targeting exon 9 of hTGFBR2. In some embodiments, the CRISPR gene-editing system for the treatment of PsA comprising one or more sgRNAs targeting an exon of hTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTGFBR2. In some embodiments, the CRISPR gene-editing system for the treatment of psoriatic arthritis comprises one or more sgRNAs targeting exon 2 of cTGFBR2. In some embodiments, the CRISPR gene-editing system for the treatment of psoriatic arthritis comprises one or more sgRNAs targeting exon 3 of cTGFBR2. In some embodiments, the CRISPR gene-editing system for the treatment of psoriatic arthritis comprises one or more sgRNAs targeting exon 4 of cTGFBR2. In some embodiments, the CRISPR gene-editing system for the treatment of psoriatic arthritis comprises one or more sgRNAs targeting exon 5 of cTGFBR2. In some embodiments, the CRISPR gene-editing system for the treatment of psoriatic arthritis comprises one or more sgRNAs targeting exon 6 of cTGFBR2. In some embodiments, the CRISPR gene-editing system for the treatment of psoriatic arthritis comprises one or more sgRNAs targeting exon 7 of cTGFBR2. In some embodiments, the CRISPR gene-editing system for the treatment of psoriatic arthritis comprises one or more sgRNAs targeting exon 8 of cTGFBR2. In some embodiments, the CRISPR gene-editing system for the treatment of PsA comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTGFBR2. In some embodiments, the CRISPR gene-editing system for the treatment of psoriatic arthritis comprises one or more sgRNAs targeting exon 2 of eTGFBR2. In some embodiments, the CRISPR gene-editing system for the treatment of psoriatic arthritis comprises one or more sgRNAs targeting exon 3 of eTGFBR2. In some embodiments, the CRISPR gene-editing system for the treatment of psoriatic arthritis comprises one or more sgRNAs targeting exon 4 of eTGFBR2. In some embodiments, the CRISPR gene-editing system for the treatment of psoriatic arthritis comprises one or more sgRNAs targeting exon 5 of eTGFBR2. In some embodiments, the CRISPR gene-editing system for the treatment of psoriatic arthritis comprises one or more sgRNAs targeting exon 6 of eTGFBR2. In some embodiments, the CRISPR gene-editing system for the treatment of psoriatic arthritis comprises one or more sgRNAs targeting exon 7 of eTGFBR2. In some embodiments, the CRISPR gene-editing system for the treatment of psoriatic arthritis comprises one or more sgRNAs targeting exon 8 of eTGFBR2. In some embodiments, the CRISPR gene-editing system for the treatment of psoriatic arthritis comprises one or more sgRNAs targeting exon 9 of eTGFBR2. In some embodiments, the CRISPR gene-editing system for the treatment of psoriatic arthritis comprises one or more sgRNAs targeting exon 10 of eTGFBR2. In some embodiments, the CRISPR gene-editing system for the treatment of PsA comprising one or more sgRNAs targeting an exon of eTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTGFBR2. In some embodiments, the CRISPR gene-editing system for the treatment of psoriatic arthritis comprises one or more sgRNAs targeting exon 2 of fTGFBR2. In some embodiments, the CRISPR gene-editing system for the treatment of psoriatic arthritis comprises one or more sgRNAs targeting exon 3 of fTGFBR2. In some embodiments, the CRISPR gene-editing system for the treatment of psoriatic arthritis comprises one or more sgRNAs targeting exon 4 of fTGFBR2. In some embodiments, the CRISPR gene-editing system for the treatment of psoriatic arthritis comprises one or more sgRNAs targeting exon 5 of fTGFBR2. In some embodiments, the CRISPR gene-editing system for the treatment of psoriatic arthritis comprises one or more sgRNAs targeting exon 6 of fTGFBR2. In some embodiments, the CRISPR gene-editing system for the treatment of psoriatic arthritis comprises one or more sgRNAs targeting exon 7 of fTGFBR2. In some embodiments, the CRISPR gene-editing system for the treatment of psoriatic arthritis comprises one or more sgRNAs targeting exon 8 of fTGFBR2. In some embodiments, the CRISPR gene-editing system for the treatment of PsA comprising one or more sgRNAs targeting an exon of fTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting IL6R. In some embodiments, the CRISPR gene-editing system for the treatment of psoriatic arthritis comprises one or more sgRNAs targeting hIL6R. In some embodiments, the CRISPR gene-editing system for the treatment of psoriatic arthritis comprises one or more sgRNAs targeting cIL6R. In some embodiments, the CRISPR gene-editing system for the treatment of psoriatic arthritis comprises one or more sgRNAs targeting eIL6R. In some embodiments, the CRISPR gene-editing system for the treatment of psoriatic arthritis comprises one or more sgRNAs targeting fIL6R. In some embodiments, the CRISPR gene-editing system for the treatment of PsA comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hIL6R. In some embodiments, the CRISPR gene-editing system for the treatment of psoriatic arthritis comprises one or more sgRNAs targeting exon 2 of hIL6R. In some embodiments, the CRISPR gene-editing system for the treatment of psoriatic arthritis comprises one or more sgRNAs targeting exon 3 of hIL6R. In some embodiments, the CRISPR gene-editing system for the treatment of psoriatic arthritis comprises one or more sgRNAs targeting exon 4 of hIL6R. In some embodiments, the CRISPR gene-editing system for the treatment of psoriatic arthritis comprises one or more sgRNAs targeting exon 5 of hIL6R. In some embodiments, the CRISPR gene-editing system for the treatment of psoriatic arthritis comprises one or more sgRNAs targeting exon 6 of hIL6R. In some embodiments, the CRISPR gene-editing system for the treatment of psoriatic arthritis comprises one or more sgRNAs targeting exon 7 of hIL6R. In some embodiments, the CRISPR gene-editing system for the treatment of psoriatic arthritis comprises one or more sgRNAs targeting exon 8 of hIL6R. In some embodiments, the CRISPR gene-editing system for the treatment of psoriatic arthritis comprises one or more sgRNAs targeting exon 9 of hIL6R. In some embodiments, the CRISPR gene-editing system for the treatment of psoriatic arthritis comprises one or more sgRNAs targeting exon 10 of hIL6R. In some embodiments, the CRISPR gene-editing system for the treatment of psoriatic arthritis comprises one or more sgRNAs targeting exon 11 of hIL6R In some embodiments, the CRISPR gene-editing system for the treatment of psoriatic arthritis comprises one or more sgRNAs targeting exon 12 of hIL6R. In some embodiments, the CRISPR gene-editing system for the treatment of psoriatic arthritis comprises one or more sgRNAs targeting exon 13 of hIL6R. In some embodiments, the CRISPR gene-editing system for the treatment of psoriatic arthritis comprises one or more sgRNAs targeting exon 14 of hIL6R. In some embodiments, the CRISPR gene-editing system for the treatment of psoriatic arthritis comprises one or more sgRNAs targeting exon 15 of hIL6R. In some embodiments, the CRISPR gene-editing system for the treatment of PsA comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cIL6R. In some embodiments, the CRISPR gene-editing system for the treatment of psoriatic arthritis comprises one or more sgRNAs targeting exon 2 of cIL6R. In some embodiments, the CRISPR gene-editing system for the treatment of psoriatic arthritis comprises one or more sgRNAs targeting exon 3 of cIL6R. In some embodiments, the CRISPR gene-editing system for the treatment of psoriatic arthritis comprises one or more sgRNAs targeting exon 4 of cIL6R. In some embodiments, the CRISPR gene-editing system for the treatment of psoriatic arthritis comprises one or more sgRNAs targeting exon 5 of cIL6R. In some embodiments, the CRISPR gene-editing system for the treatment of psoriatic arthritis comprises one or more sgRNAs targeting exon 6 of cIL6R. In some embodiments, the CRISPR gene-editing system for the treatment of psoriatic arthritis comprises one or more sgRNAs targeting exon 7 of cIL6R. In some embodiments, the CRISPR gene-editing system for the treatment of psoriatic arthritis comprises one or more sgRNAs targeting exon 8 of cIL6R. In some embodiments, the CRISPR gene-editing system for the treatment of psoriatic arthritis comprises one or more sgRNAs targeting exon 9 of cIL6R. In some embodiments, the CRISPR gene-editing system for the treatment of psoriatic arthritis comprises one or more sgRNAs targeting exon 10 of cIL6R. In some embodiments, the CRISPR gene-editing system for the treatment of psoriatic arthritis comprises one or more sgRNAs targeting exon 11 of cIL6R. In some embodiments, the CRISPR gene-editing system for the treatment of psoriatic arthritis comprises one or more sgRNAs targeting exon 12 of cIL6R. In some embodiments, the CRISPR gene-editing system for the treatment of psoriatic arthritis comprises one or more sgRNAs targeting exon 13 of cIL6R. In some embodiments, the CRISPR gene-editing system for the treatment of PsA comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eIL6R. In some embodiments, the CRISPR gene-editing system for the treatment of psoriatic arthritis comprises one or more sgRNAs targeting exon 2 of eIL6R. In some embodiments, the CRISPR gene-editing system for the treatment of psoriatic arthritis comprises one or more sgRNAs targeting exon 3 of eIL6R. In some embodiments, the CRISPR gene-editing system for the treatment of psoriatic arthritis comprises one or more sgRNAs targeting exon 4 of eIL6R. In some embodiments, the CRISPR gene-editing system for the treatment of psoriatic arthritis comprises one or more sgRNAs targeting exon 5 of eIL6R. In some embodiments, the CRISPR gene-editing system for the treatment of psoriatic arthritis comprises one or more sgRNAs targeting exon 6 of eIL6R. In some embodiments, the CRISPR gene-editing system for the treatment of psoriatic arthritis comprises one or more sgRNAs targeting exon 7 of eIL6R. In some embodiments, the CRISPR gene-editing system for the treatment of psoriatic arthritis comprises one or more sgRNAs targeting exon 8 of eIL6R. In some embodiments, the CRISPR gene-editing system for the treatment of psoriatic arthritis comprises one or more sgRNAs targeting exon 9 of eIL6R. In some embodiments, the CRISPR gene-editing system for the treatment of psoriatic arthritis comprises one or more sgRNAs targeting exon 10 of eIL6R. In some embodiments, the CRISPR gene-editing system for the treatment of psoriatic arthritis comprises one or more sgRNAs targeting exon 11 of eIL6R. In some embodiments, the CRISPR gene-editing system for the treatment of PsA comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fIL6R. In some embodiments, the CRISPR gene-editing system for the treatment of psoriatic arthritis comprises one or more sgRNAs targeting exon 2 of fIL6R. In some embodiments, the CRISPR gene-editing system for the treatment of psoriatic arthritis comprises one or more sgRNAs targeting exon 3 of fIL6R. In some embodiments, the CRISPR gene-editing system for the treatment of psoriatic arthritis comprises one or more sgRNAs targeting exon 4 of fIL6R. In some embodiments, the CRISPR gene-editing system for the treatment of psoriatic arthritis comprises one or more sgRNAs targeting exon 5 of fIL6R. In some embodiments, the CRISPR gene-editing system for the treatment of psoriatic arthritis comprises one or more sgRNAs targeting exon 6 of fIL6R. In some embodiments, the CRISPR gene-editing system for the treatment of psoriatic arthritis comprises one or more sgRNAs targeting exon 7 of fIL6R. In some embodiments, the CRISPR gene-editing system for the treatment of psoriatic arthritis comprises one or more sgRNAs targeting exon 8 of fIL6R. In some embodiments, the CRISPR gene-editing system for the treatment of psoriatic arthritis comprises one or more sgRNAs targeting exon 9 of fIL6R. In some embodiments, the CRISPR gene-editing system for the treatment of psoriatic arthritis comprises one or more sgRNAs targeting exon 10 of fIL6R. In some embodiments, the CRISPR gene-editing system for the treatment of PsA comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting IL6ST. In some embodiments, the CRISPR gene-editing system for the treatment of psoriatic arthritis comprises one or more sgRNAs targeting hIL6ST. In some embodiments, the CRISPR gene-editing system for the treatment of psoriatic arthritis comprises one or more sgRNAs targeting cIL6ST. In some embodiments, the CRISPR gene-editing system for the treatment of psoriatic arthritis comprises one or more sgRNAs targeting eIL6ST. In some embodiments, the CRISPR gene-editing system for the treatment of psoriatic arthritis comprises one or more sgRNAs targeting fIL6ST. In some embodiments, the CRISPR gene-editing system for the treatment of PsA comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hIL6ST. In some embodiments, the CRISPR gene-editing system for the treatment of psoriatic arthritis comprises one or more sgRNAs targeting exon 2 of hIL6ST. In some embodiments, the CRISPR gene-editing system for the treatment of psoriatic arthritis comprises one or more sgRNAs targeting exon 3 of hIL6ST. In some embodiments, the CRISPR gene-editing system for the treatment of psoriatic arthritis comprises one or more sgRNAs targeting exon 4 of hIL6ST. In some embodiments, the CRISPR gene-editing system for the treatment of psoriatic arthritis comprises one or more sgRNAs targeting exon 5 of hIL6ST. In some embodiments, the CRISPR gene-editing system for the treatment of psoriatic arthritis comprises one or more sgRNAs targeting exon 6 of hIL6ST. In some embodiments, the CRISPR gene-editing system for the treatment of psoriatic arthritis comprises one or more sgRNAs targeting exon 7 of hIL6ST. In some embodiments, the CRISPR gene-editing system for the treatment of psoriatic arthritis comprises one or more sgRNAs targeting exon 8 of hIL6ST. In some embodiments, the CRISPR gene-editing system for the treatment of psoriatic arthritis comprises one or more sgRNAs targeting exon 9 of hIL6ST. In some embodiments, the CRISPR gene-editing system for the treatment of psoriatic arthritis comprises one or more sgRNAs targeting exon 10 of hIL6ST. In some embodiments, the CRISPR gene-editing system for the treatment of psoriatic arthritis comprises one or more sgRNAs targeting exon 11 of hIL6ST. In some embodiments, the CRISPR gene-editing system for the treatment of psoriatic arthritis comprises one or more sgRNAs targeting exon 12 of hIL6ST. In some embodiments, the CRISPR gene-editing system for the treatment of psoriatic arthritis comprises one or more sgRNAs targeting exon 13 of hIL6ST. In some embodiments, the CRISPR gene-editing system for the treatment of psoriatic arthritis comprises one or more sgRNAs targeting exon 14 of hIL6ST. In some embodiments, the CRISPR gene-editing system for the treatment of psoriatic arthritis comprises one or more sgRNAs targeting exon 15 of hIL6ST. In some embodiments, the CRISPR gene-editing system for the treatment of psoriatic arthritis comprises one or more sgRNAs targeting exon 16 of hIL6ST. In some embodiments, the CRISPR gene-editing system for the treatment of psoriatic arthritis comprises one or more sgRNAs targeting exon 17 of hIL6ST. In some embodiments, the CRISPR gene-editing system for the treatment of psoriatic arthritis comprises one or more sgRNAs targeting exon 18 of hIL6ST. In some embodiments, the CRISPR gene-editing system for the treatment of psoriatic arthritis comprises one or more sgRNAs targeting exon 19 of hIL6ST. In some embodiments, the CRISPR gene-editing system for the treatment of PsA comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cIL6ST. In some embodiments, the CRISPR gene-editing system for the treatment of psoriatic arthritis comprises one or more sgRNAs targeting exon 2 of cIL6ST. In some embodiments, the CRISPR gene-editing system for the treatment of psoriatic arthritis comprises one or more sgRNAs targeting exon 3 of cIL6ST. In some embodiments, the CRISPR gene-editing system for the treatment of psoriatic arthritis comprises one or more sgRNAs targeting exon 4 of cIL6ST. In some embodiments, the CRISPR gene-editing system for the treatment of psoriatic arthritis comprises one or more sgRNAs targeting exon 5 of cIL6ST. In some embodiments, the CRISPR gene-editing system for the treatment of psoriatic arthritis comprises one or more sgRNAs targeting exon 6 of cIL6ST. In some embodiments, the CRISPR gene-editing system for the treatment of psoriatic arthritis comprises one or more sgRNAs targeting exon 7 of cIL6ST. In some embodiments, the CRISPR gene-editing system for the treatment of psoriatic arthritis comprises one or more sgRNAs targeting exon 8 of cIL6ST. In some embodiments, the CRISPR gene-editing system for the treatment of psoriatic arthritis comprises one or more sgRNAs targeting exon 9 of cIL6ST. In some embodiments, the CRISPR gene-editing system for the treatment of psoriatic arthritis comprises one or more sgRNAs targeting exon 10 of cIL6ST. In some embodiments, the CRISPR gene-editing system for the treatment of psoriatic arthritis comprises one or more sgRNAs targeting exon 11 of cIL6ST In some embodiments, the CRISPR gene-editing system for the treatment of psoriatic arthritis comprises one or more sgRNAs targeting exon 12 of cIL6ST. In some embodiments, the CRISPR gene-editing system for the treatment of psoriatic arthritis comprises one or more sgRNAs targeting exon 13 of cIL6ST. In some embodiments, the CRISPR gene-editing system for the treatment of psoriatic arthritis comprises one or more sgRNAs targeting exon 14 of cIL6ST. In some embodiments, the CRISPR gene-editing system for the treatment of psoriatic arthritis comprises one or more sgRNAs targeting exon 15 of cIL6ST. In some embodiments, the CRISPR gene-editing system for the treatment of psoriatic arthritis comprises one or more sgRNAs targeting exon 16 of cIL6ST. In some embodiments, the CRISPR gene-editing system for the treatment of psoriatic arthritis comprises one or more sgRNAs targeting exon 17 of cIL6ST. In some embodiments, the CRISPR gene-editing system for the treatment of psoriatic arthritis comprises one or more sgRNAs targeting exon 18 of cIL6ST. In some embodiments, the CRISPR gene-editing system for the treatment of PsA comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eIL6ST. In some embodiments, the CRISPR gene-editing system for the treatment of psoriatic arthritis comprises one or more sgRNAs targeting exon 2 of eIL6ST. In some embodiments, the CRISPR gene-editing system for the treatment of psoriatic arthritis comprises one or more sgRNAs targeting exon 3 of eIL6ST. In some embodiments, the CRISPR gene-editing system for the treatment of psoriatic arthritis comprises one or more sgRNAs targeting exon 4 of eIL6ST. In some embodiments, the CRISPR gene-editing system for the treatment of psoriatic arthritis comprises one or more sgRNAs targeting exon 5 of eIL6ST. In some embodiments, the CRISPR gene-editing system for the treatment of psoriatic arthritis comprises one or more sgRNAs targeting exon 6 of eIL6ST. In some embodiments, the CRISPR gene-editing system for the treatment of psoriatic arthritis comprises one or more sgRNAs targeting exon 7 of eIL6ST. In some embodiments, the CRISPR gene-editing system for the treatment of psoriatic arthritis comprises one or more sgRNAs targeting exon 8 of eIL6ST. In some embodiments, the CRISPR gene-editing system for the treatment of psoriatic arthritis comprises one or more sgRNAs targeting exon 9 of eIL6ST. In some embodiments, the CRISPR gene-editing system for the treatment of psoriatic arthritis comprises one or more sgRNAs targeting exon 10 of eIL6ST. In some embodiments, the CRISPR gene-editing system for the treatment of psoriatic arthritis comprises one or more sgRNAs targeting exon 11 of eIL6ST In some embodiments, the CRISPR gene-editing system for the treatment of psoriatic arthritis comprises one or more sgRNAs targeting exon 12 of eIL6ST. In some embodiments, the CRISPR gene-editing system for the treatment of psoriatic arthritis comprises one or more sgRNAs targeting exon 13 of eIL6ST. In some embodiments, the CRISPR gene-editing system for the treatment of psoriatic arthritis comprises one or more sgRNAs targeting exon 14 of eIL6ST. In some embodiments, the CRISPR gene-editing system for the treatment of psoriatic arthritis comprises one or more sgRNAs targeting exon 15 of eIL6ST. In some embodiments, the CRISPR gene-editing system for the treatment of psoriatic arthritis comprises one or more sgRNAs targeting exon 16 of eIL6ST. In some embodiments, the CRISPR gene-editing system for the treatment of psoriatic arthritis comprises one or more sgRNAs targeting exon 17 of eIL6ST. In some embodiments, the CRISPR gene-editing system for the treatment of psoriatic arthritis comprises one or more sgRNAs targeting exon 18 of eIL6ST. In some embodiments, the CRISPR gene-editing system for the treatment of PsA comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fIL6ST. In some embodiments, the CRISPR gene-editing system for the treatment of psoriatic arthritis comprises one or more sgRNAs targeting exon 2 of fIL6ST. In some embodiments, the CRISPR gene-editing system for the treatment of psoriatic arthritis comprises one or more sgRNAs targeting exon 3 of fIL6ST. In some embodiments, the CRISPR gene-editing system for the treatment of psoriatic arthritis comprises one or more sgRNAs targeting exon 4 of fIL6ST. In some embodiments, the CRISPR gene-editing system for the treatment of psoriatic arthritis comprises one or more sgRNAs targeting exon 5 of fIL6ST. In some embodiments, the CRISPR gene-editing system for the treatment of psoriatic arthritis comprises one or more sgRNAs targeting exon 6 of fIL6ST. In some embodiments, the CRISPR gene-editing system for the treatment of psoriatic arthritis comprises one or more sgRNAs targeting exon 7 of fIL6ST. In some embodiments, the CRISPR gene-editing system for the treatment of psoriatic arthritis comprises one or more sgRNAs targeting exon 8 of fIL6ST. In some embodiments, the CRISPR gene-editing system for the treatment of psoriatic arthritis comprises one or more sgRNAs targeting exon 9 of fIL6ST. In some embodiments, the CRISPR gene-editing system for the treatment of psoriatic arthritis comprises one or more sgRNAs targeting exon 10 of fIL6ST. In some embodiments, the CRISPR gene-editing system for the treatment of psoriatic arthritis comprises one or more sgRNAs targeting exon 11 of fIL6ST In some embodiments, the CRISPR gene-editing system for the treatment of psoriatic arthritis comprises one or more sgRNAs targeting exon 12 of fIL6ST. In some embodiments, the CRISPR gene-editing system for the treatment of psoriatic arthritis comprises one or more sgRNAs targeting exon 13 of fIL6ST. In some embodiments, the CRISPR gene-editing system for the treatment of psoriatic arthritis comprises one or more sgRNAs targeting exon 14 of fIL6ST. In some embodiments, the CRISPR gene-editing system for the treatment of psoriatic arthritis comprises one or more sgRNAs targeting exon 15 of fIL6ST. In some embodiments, the CRISPR gene-editing system for the treatment of psoriatic arthritis comprises one or more sgRNAs targeting exon 16 of fIL6ST. In some embodiments, the CRISPR gene-editing system for the treatment of psoriatic arthritis comprises one or more sgRNAs targeting exon 17 of fIL6ST. In some embodiments, the CRISPR gene-editing system for the treatment of psoriatic arthritis comprises one or more sgRNAs targeting exon 18 of fIL6ST. In some embodiments, the CRISPR gene-editing system for the treatment of psoriatic arthritis comprises one or more sgRNAs targeting exon 19 of fIL6ST. In some embodiments, the CRISPR gene-editing system for the treatment of psoriatic arthritis comprises one or more sgRNAs targeting exon 20 of fIL6ST. In some embodiments, the CRISPR gene-editing system for the treatment of PsA comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TNFRSF1A. In some embodiments, the CRISPR gene-editing system for the treatment of psoriatic arthritis comprises one or more sgRNAs targeting hTNFRSF1A. In some embodiments, the CRISPR gene-editing system for the treatment of psoriatic arthritis comprises one or more sgRNAs targeting cTNFRSF1A. In some embodiments, the CRISPR gene-editing system for the treatment of psoriatic arthritis comprises one or more sgRNAs targeting eTNFRSF1A. In some embodiments, the CRISPR gene-editing system for the treatment of psoriatic arthritis comprises one or more sgRNAs targeting fTNFRSF1A. In some embodiments, the CRISPR gene-editing system for the treatment of PsA comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTNFRSF1A. In some embodiments, the CRISPR gene-editing system for the treatment of psoriatic arthritis comprises one or more sgRNAs targeting exon 2 of hTNFRSF1A. In some embodiments, the CRISPR gene-editing system for the treatment of psoriatic arthritis comprises one or more sgRNAs targeting exon 3 of hTNFRSF1A. In some embodiments, the CRISPR gene-editing system for the treatment of psoriatic arthritis comprises one or more sgRNAs targeting exon 4 of hTNFRSF1A. In some embodiments, the CRISPR gene-editing system for the treatment of psoriatic arthritis comprises one or more sgRNAs targeting exon 5 of hTNFRSF1A. In some embodiments, the CRISPR gene-editing system for the treatment of psoriatic arthritis comprises one or more sgRNAs targeting exon 6 of hTNFRSF1A. In some embodiments, the CRISPR gene-editing system for the treatment of psoriatic arthritis comprises one or more sgRNAs targeting exon 7 of hTNFRSF1A. In some embodiments, the CRISPR gene-editing system for the treatment of psoriatic arthritis comprises one or more sgRNAs targeting exon 8 of hTNFRSF1A. In some embodiments, the CRISPR gene-editing system for the treatment of psoriatic arthritis comprises one or more sgRNAs targeting exon 9 of hTNFRSF1A. In some embodiments, the CRISPR gene-editing system for the treatment of psoriatic arthritis comprises one or more sgRNAs targeting exon 10 of hTNFRSF1A. In some embodiments, the CRISPR gene-editing system for the treatment of psoriatic arthritis comprises one or more sgRNAs targeting exon 11 of hTNFRSF1A. In some embodiments, the CRISPR gene-editing system for the treatment of PsA comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTNFRSF1A. In some embodiments, the CRISPR gene-editing system for the treatment of psoriatic arthritis comprises one or more sgRNAs targeting exon 2 of cTNFRSF1A. In some embodiments, the CRISPR gene-editing system for the treatment of psoriatic arthritis comprises one or more sgRNAs targeting exon 3 of cTNFRSF1A. In some embodiments, the CRISPR gene-editing system for the treatment of psoriatic arthritis comprises one or more sgRNAs targeting exon 4 of cTNFRSF1A. In some embodiments, the CRISPR gene-editing system for the treatment of psoriatic arthritis comprises one or more sgRNAs targeting exon 5 of cTNFRSF1A. In some embodiments, the CRISPR gene-editing system for the treatment of psoriatic arthritis comprises one or more sgRNAs targeting exon 6 of cTNFRSF1A. In some embodiments, the CRISPR gene-editing system for the treatment of psoriatic arthritis comprises one or more sgRNAs targeting exon 7 of cTNFRSF1A. In some embodiments, the CRISPR gene-editing system for the treatment of psoriatic arthritis comprises one or more sgRNAs targeting exon 8 of cTNFRSF1A. In some embodiments, the CRISPR gene-editing system for the treatment of psoriatic arthritis comprises one or more sgRNAs targeting exon 9 of cTNFRSF1A. In some embodiments, the CRISPR gene-editing system for the treatment of psoriatic arthritis comprises one or more sgRNAs targeting exon 10 of cTNFRSF1A. In some embodiments, the CRISPR gene-editing system for the treatment of psoriatic arthritis comprises one or more sgRNAs targeting exon 11 of cTNFRSF1A. In some embodiments, the CRISPR gene-editing system for the treatment of PsA comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTNFRSF1A. In some embodiments, the CRISPR gene-editing system for the treatment of psoriatic arthritis comprises one or more sgRNAs targeting exon 2 of eTNFRSF1A. In some embodiments, the CRISPR gene-editing system for the treatment of psoriatic arthritis comprises one or more sgRNAs targeting exon 3 of eTNFRSF1A. In some embodiments, the CRISPR gene-editing system for the treatment of psoriatic arthritis comprises one or more sgRNAs targeting exon 4 of eTNFRSF1A. In some embodiments, the CRISPR gene-editing system for the treatment of psoriatic arthritis comprises one or more sgRNAs targeting exon 5 of eTNFRSF1A. In some embodiments, the CRISPR gene-editing system for the treatment of psoriatic arthritis comprises one or more sgRNAs targeting exon 6 of eTNFRSF1A. In some embodiments, the CRISPR gene-editing system for the treatment of psoriatic arthritis comprises one or more sgRNAs targeting exon 7 of eTNFRSF1A. In some embodiments, the CRISPR gene-editing system for the treatment of psoriatic arthritis comprises one or more sgRNAs targeting exon 8 of eTNFRSF1A. In some embodiments, the CRISPR gene-editing system for the treatment of psoriatic arthritis comprises one or more sgRNAs targeting exon 9 of eTNFRSF1A. In some embodiments, the CRISPR gene-editing system for the treatment of psoriatic arthritis comprises one or more sgRNAs targeting exon 10 of eTNFRSF1A. In some embodiments, the CRISPR gene-editing system for the treatment of PsA comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTNFRSF1A. In some embodiments, the CRISPR gene-editing system for the treatment of psoriatic arthritis comprises one or more sgRNAs targeting exon 2 of fTNFRSF1A. In some embodiments, the CRISPR gene-editing system for the treatment of psoriatic arthritis comprises one or more sgRNAs targeting exon 3 of fTNFRSF1A. In some embodiments, the CRISPR gene-editing system for the treatment of psoriatic arthritis comprises one or more sgRNAs targeting exon 4 of fTNFRSF1A. In some embodiments, the CRISPR gene-editing system for the treatment of psoriatic arthritis comprises one or more sgRNAs targeting exon 5 of fTNFRSF1A. In some embodiments, the CRISPR gene-editing system for the treatment of psoriatic arthritis comprises one or more sgRNAs targeting exon 6 of fTNFRSF1A. In some embodiments, the CRISPR gene-editing system for the treatment of psoriatic arthritis comprises one or more sgRNAs targeting exon 7 of fTNFRSF1A. In some embodiments, the CRISPR gene-editing system for the treatment of psoriatic arthritis comprises one or more sgRNAs targeting exon 8 of fTNFRSF1A. In some embodiments, the CRISPR gene-editing system for the treatment of psoriatic arthritis comprises one or more sgRNAs targeting exon 9 of fTNFRSF1A. In some embodiments, the CRISPR gene-editing system for the treatment of psoriatic arthritis comprises one or more sgRNAs targeting exon 10 of fTNFRSF1A. In some embodiments, the CRISPR gene-editing system for the treatment of PsA comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TNFRSF1B. In some embodiments, the CRISPR gene-editing system for the treatment of psoriatic arthritis comprises one or more sgRNAs targeting hTNFRSF1B. In some embodiments, the CRISPR gene-editing system for the treatment of psoriatic arthritis comprises one or more sgRNAs targeting cTNFRSF1B. In some embodiments, the CRISPR gene-editing system for the treatment of psoriatic arthritis comprises one or more sgRNAs targeting eTNFRSF1B. In some embodiments, the CRISPR gene-editing system for the treatment of psoriatic arthritis comprises one or more sgRNAs targeting fTNFRSF1B. In some embodiments, the CRISPR gene-editing system for the treatment of PsA comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTNFRSF1B. In some embodiments, the CRISPR gene-editing system for the treatment of psoriatic arthritis comprises one or more sgRNAs targeting exon 2 of hTNFRSF1B. In some embodiments, the CRISPR gene-editing system for the treatment of psoriatic arthritis comprises one or more sgRNAs targeting exon 3 of hTNFRSF1B. In some embodiments, the CRISPR gene-editing system for the treatment of psoriatic arthritis comprises one or more sgRNAs targeting exon 4 of hTNFRSF1B. In some embodiments, the CRISPR gene-editing system for the treatment of psoriatic arthritis comprises one or more sgRNAs targeting exon 5 of hTNFRSF1B. In some embodiments, the CRISPR gene-editing system for the treatment of psoriatic arthritis comprises one or more sgRNAs targeting exon 6 of hTNFRSF1B. In some embodiments, the CRISPR gene-editing system for the treatment of psoriatic arthritis comprises one or more sgRNAs targeting exon 7 of hTNFRSF1B. In some embodiments, the CRISPR gene-editing system for the treatment of psoriatic arthritis comprises one or more sgRNAs targeting exon 8 of hTNFRSF1B. In some embodiments, the CRISPR gene-editing system for the treatment of psoriatic arthritis comprises one or more sgRNAs targeting exon 9 of hTNFRSF1B. In some embodiments, the CRISPR gene-editing system for the treatment of psoriatic arthritis comprises one or more sgRNAs targeting exon 10 of hTNFRSF1B. In some embodiments, the CRISPR gene-editing system for the treatment of psoriatic arthritis comprises one or more sgRNAs targeting exon 11 of hTNFRSF1B In some embodiments, the CRISPR gene-editing system for the treatment of psoriatic arthritis comprises one or more sgRNAs targeting exon 12 of hTNFRSF1B. In some embodiments, the CRISPR gene-editing system for the treatment of psoriatic arthritis comprises one or more sgRNAs targeting exon 13 of hTNFRSF1B. In some embodiments, the CRISPR gene-editing system for the treatment of PsA comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTNFRSF1B. In some embodiments, the CRISPR gene-editing system for the treatment of psoriatic arthritis comprises one or more sgRNAs targeting exon 2 of cTNFRSF1B. In some embodiments, the CRISPR gene-editing system for the treatment of psoriatic arthritis comprises one or more sgRNAs targeting exon 3 of cTNFRSF1B. In some embodiments, the CRISPR gene-editing system for the treatment of psoriatic arthritis comprises one or more sgRNAs targeting exon 4 of cTNFRSF1B. In some embodiments, the CRISPR gene-editing system for the treatment of psoriatic arthritis comprises one or more sgRNAs targeting exon 5 of cTNFRSF1B. In some embodiments, the CRISPR gene-editing system for the treatment of psoriatic arthritis comprises one or more sgRNAs targeting exon 6 of cTNFRSF1B. In some embodiments, the CRISPR gene-editing system for the treatment of psoriatic arthritis comprises one or more sgRNAs targeting exon 7 of cTNFRSF1B. In some embodiments, the CRISPR gene-editing system for the treatment of psoriatic arthritis comprises one or more sgRNAs targeting exon 8 of cTNFRSF1B. In some embodiments, the CRISPR gene-editing system for the treatment of psoriatic arthritis comprises one or more sgRNAs targeting exon 9 of cTNFRSF1B. In some embodiments, the CRISPR gene-editing system for the treatment of psoriatic arthritis comprises one or more sgRNAs targeting exon 10 of cTNFRSF1B. In some embodiments, the CRISPR gene-editing system for the treatment of psoriatic arthritis comprises one or more sgRNAs targeting exon 11 of cTNFRSF1B. In some embodiments, the CRISPR gene-editing system for the treatment of PsA comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTNFRSF1B. In some embodiments, the CRISPR gene-editing system for the treatment of psoriatic arthritis comprises one or more sgRNAs targeting exon 2 of eTNFRSF1B. In some embodiments, the CRISPR gene-editing system for the treatment of psoriatic arthritis comprises one or more sgRNAs targeting exon 3 of eTNFRSF1B. In some embodiments, the CRISPR gene-editing system for the treatment of psoriatic arthritis comprises one or more sgRNAs targeting exon 4 of eTNFRSF1B. In some embodiments, the CRISPR gene-editing system for the treatment of psoriatic arthritis comprises one or more sgRNAs targeting exon 5 of eTNFRSF1B. In some embodiments, the CRISPR gene-editing system for the treatment of psoriatic arthritis comprises one or more sgRNAs targeting exon 6 of eTNFRSF1B. In some embodiments, the CRISPR gene-editing system for the treatment of psoriatic arthritis comprises one or more sgRNAs targeting exon 7 of eTNFRSF1B. In some embodiments, the CRISPR gene-editing system for the treatment of psoriatic arthritis comprises one or more sgRNAs targeting exon 8 of eTNFRSF1B. In some embodiments, the CRISPR gene-editing system for the treatment of psoriatic arthritis comprises one or more sgRNAs targeting exon 9 of eTNFRSF1B. In some embodiments, the CRISPR gene-editing system for the treatment of psoriatic arthritis comprises one or more sgRNAs targeting exon 10 of eTNFRSF1B. In some embodiments, the CRISPR gene-editing system for the treatment of psoriatic arthritis comprises one or more sgRNAs targeting exon 11 of eTNFRSF1B. In some embodiments, the CRISPR gene-editing system for the treatment of PsA comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTNFRSF1B. In some embodiments, the CRISPR gene-editing system for the treatment of psoriatic arthritis comprises one or more sgRNAs targeting exon 2 of fTNFRSF1B. In some embodiments, the CRISPR gene-editing system for the treatment of psoriatic arthritis comprises one or more sgRNAs targeting exon 3 of fTNFRSF1B. In some embodiments, the CRISPR gene-editing system for the treatment of psoriatic arthritis comprises one or more sgRNAs targeting exon 4 of fTNFRSF1B. In some embodiments, the CRISPR gene-editing system for the treatment of psoriatic arthritis comprises one or more sgRNAs targeting exon 5 of fTNFRSF1B. In some embodiments, the CRISPR gene-editing system for the treatment of psoriatic arthritis comprises one or more sgRNAs targeting exon 6 of fTNFRSF1B. In some embodiments, the CRISPR gene-editing system for the treatment of psoriatic arthritis comprises one or more sgRNAs targeting exon 7 of fTNFRSF1B. In some embodiments, the CRISPR gene-editing system for the treatment of psoriatic arthritis comprises one or more sgRNAs targeting exon 8 of fTNFRSF1B. In some embodiments, the CRISPR gene-editing system for the treatment of psoriatic arthritis comprises one or more sgRNAs targeting exon 9 of fTNFRSF1B. In some embodiments, the CRISPR gene-editing system for the treatment of psoriatic arthritis comprises one or more sgRNAs targeting exon 10 of fTNFRSF1B. In some embodiments, the CRISPR gene-editing system for the treatment of PsA comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TNFRSF3. In some embodiments, the CRISPR gene-editing system for the treatment of psoriatic arthritis comprises one or more sgRNAs targeting hTNFRSF3. In some embodiments, the CRISPR gene-editing system for the treatment of psoriatic arthritis comprises one or more sgRNAs targeting cTNFRSF3. In some embodiments, the CRISPR gene-editing system for the treatment of psoriatic arthritis comprises one or more sgRNAs targeting eTNFRSF3. In some embodiments, the CRISPR gene-editing system for the treatment of psoriatic arthritis comprises one or more sgRNAs targeting fTNFRSF3. In some embodiments, the CRISPR gene-editing system for the treatment of PsA comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTNFRSF3. In some embodiments, the CRISPR gene-editing system for the treatment of psoriatic arthritis comprises one or more sgRNAs targeting exon 2 of hTNFRSF3. In some embodiments, the CRISPR gene-editing system for the treatment of psoriatic arthritis comprises one or more sgRNAs targeting exon 3 of hTNFRSF3. In some embodiments, the CRISPR gene-editing system for the treatment of psoriatic arthritis comprises one or more sgRNAs targeting exon 4 of hTNFRSF3. In some embodiments, the CRISPR gene-editing system for the treatment of psoriatic arthritis comprises one or more sgRNAs targeting exon 5 of hTNFRSF3. In some embodiments, the CRISPR gene-editing system for the treatment of psoriatic arthritis comprises one or more sgRNAs targeting exon 6 of hTNFRSF3. In some embodiments, the CRISPR gene-editing system for the treatment of psoriatic arthritis comprises one or more sgRNAs targeting exon 7 of hTNFRSF3. In some embodiments, the CRISPR gene-editing system for the treatment of psoriatic arthritis comprises one or more sgRNAs targeting exon 8 of hTNFRSF3. In some embodiments, the CRISPR gene-editing system for the treatment of psoriatic arthritis comprises one or more sgRNAs targeting exon 9 of hTNFRSF3. In some embodiments, the CRISPR gene-editing system for the treatment of psoriatic arthritis comprises one or more sgRNAs targeting exon 10 of hTNFRSF3. In some embodiments, the CRISPR gene-editing system for the treatment of psoriatic arthritis comprises one or more sgRNAs targeting exon 11 of hTNFRSF3. In some embodiments, the CRISPR gene-editing system for the treatment of psoriatic arthritis comprises one or more sgRNAs targeting exon 12 of hTNFRSF3. In some embodiments, the CRISPR gene-editing system for the treatment of PsA comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTNFRSF3. In some embodiments, the CRISPR gene-editing system for the treatment of psoriatic arthritis comprises one or more sgRNAs targeting exon 2 of cTNFRSF3. In some embodiments, the CRISPR gene-editing system for the treatment of psoriatic arthritis comprises one or more sgRNAs targeting exon 3 of cTNFRSF3. In some embodiments, the CRISPR gene-editing system for the treatment of psoriatic arthritis comprises one or more sgRNAs targeting exon 4 of cTNFRSF3. In some embodiments, the CRISPR gene-editing system for the treatment of psoriatic arthritis comprises one or more sgRNAs targeting exon 5 of cTNFRSF3. In some embodiments, the CRISPR gene-editing system for the treatment of psoriatic arthritis comprises one or more sgRNAs targeting exon 6 of cTNFRSF3. In some embodiments, the CRISPR gene-editing system for the treatment of psoriatic arthritis comprises one or more sgRNAs targeting exon 7 of cTNFRSF3. In some embodiments, the CRISPR gene-editing system for the treatment of psoriatic arthritis comprises one or more sgRNAs targeting exon 8 of cTNFRSF3. In some embodiments, the CRISPR gene-editing system for the treatment of psoriatic arthritis comprises one or more sgRNAs targeting exon 9 of cTNFRSF3. In some embodiments, the CRISPR gene-editing system for the treatment of psoriatic arthritis comprises one or more sgRNAs targeting exon 10 of cTNFRSF3. In some embodiments, the CRISPR gene-editing system for the treatment of PsA comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTNFRSF3. In some embodiments, the CRISPR gene-editing system for the treatment of psoriatic arthritis comprises one or more sgRNAs targeting exon 2 of eTNFRSF3. In some embodiments, the CRISPR gene-editing system for the treatment of psoriatic arthritis comprises one or more sgRNAs targeting exon 3 of eTNFRSF3. In some embodiments, the CRISPR gene-editing system for the treatment of psoriatic arthritis comprises one or more sgRNAs targeting exon 4 of eTNFRSF3. In some embodiments, the CRISPR gene-editing system for the treatment of psoriatic arthritis comprises one or more sgRNAs targeting exon 5 of eTNFRSF3. In some embodiments, the CRISPR gene-editing system for the treatment of psoriatic arthritis comprises one or more sgRNAs targeting exon 6 of eTNFRSF3. In some embodiments, the CRISPR gene-editing system for the treatment of psoriatic arthritis comprises one or more sgRNAs targeting exon 7 of eTNFRSF3. In some embodiments, the CRISPR gene-editing system for the treatment of psoriatic arthritis comprises one or more sgRNAs targeting exon 8 of eTNFRSF3. In some embodiments, the CRISPR gene-editing system for the treatment of psoriatic arthritis comprises one or more sgRNAs targeting exon 9 of eTNFRSF3. In some embodiments, the CRISPR gene-editing system for the treatment of psoriatic arthritis comprises one or more sgRNAs targeting exon 10 of eTNFRSF3. In some embodiments, the CRISPR gene-editing system for the treatment of PsA comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTNFRSF3. In some embodiments, the CRISPR gene-editing system for the treatment of psoriatic arthritis comprises one or more sgRNAs targeting exon 2 of fTNFRSF3. In some embodiments, the CRISPR gene-editing system for the treatment of psoriatic arthritis comprises one or more sgRNAs targeting exon 3 of fTNFRSF3. In some embodiments, the CRISPR gene-editing system for the treatment of psoriatic arthritis comprises one or more sgRNAs targeting exon 4 of fTNFRSF3. In some embodiments, the CRISPR gene-editing system for the treatment of psoriatic arthritis comprises one or more sgRNAs targeting exon 5 of fTNFRSF3. In some embodiments, the CRISPR gene-editing system for the treatment of psoriatic arthritis comprises one or more sgRNAs targeting exon 6 of fTNFRSF3. In some embodiments, the CRISPR gene-editing system for the treatment of psoriatic arthritis comprises one or more sgRNAs targeting exon 7 of fTNFRSF3. In some embodiments, the CRISPR gene-editing system for the treatment of psoriatic arthritis comprises one or more sgRNAs targeting exon 8 of fTNFRSF3. In some embodiments, the CRISPR gene-editing system for the treatment of psoriatic arthritis comprises one or more sgRNAs targeting exon 9 of fTNFRSF3. In some embodiments, the CRISPR gene-editing system for the treatment of psoriatic arthritis comprises one or more sgRNAs targeting exon 10 of fTNFRSF3. In some embodiments, the CRISPR gene-editing system for the treatment of PsA comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TNFRSF4. In some embodiments, the CRISPR gene-editing system for the treatment of psoriatic arthritis comprises one or more sgRNAs targeting hTNFRSF4. In some embodiments, the CRISPR gene-editing system for the treatment of psoriatic arthritis comprises one or more sgRNAs targeting cTNFRSF4. In some embodiments, the CRISPR gene-editing system for the treatment of psoriatic arthritis comprises one or more sgRNAs targeting eTNFRSF4. In some embodiments, the CRISPR gene-editing system for the treatment of psoriatic arthritis comprises one or more sgRNAs targeting fTNFRSF4. In some embodiments, the CRISPR gene-editing system for the treatment of PsA comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTNFRSF4. In some embodiments, the CRISPR gene-editing system for the treatment of psoriatic arthritis comprises one or more sgRNAs targeting exon 2 of hTNFRSF4. In some embodiments, the CRISPR gene-editing system for the treatment of psoriatic arthritis comprises one or more sgRNAs targeting exon 3 of hTNFRSF4. In some embodiments, the CRISPR gene-editing system for the treatment of psoriatic arthritis comprises one or more sgRNAs targeting exon 4 of hTNFRSF4. In some embodiments, the CRISPR gene-editing system for the treatment of psoriatic arthritis comprises one or more sgRNAs targeting exon 5 of hTNFRSF4. In some embodiments, the CRISPR gene-editing system for the treatment of psoriatic arthritis comprises one or more sgRNAs targeting exon 6 of hTNFRSF4. In some embodiments, the CRISPR gene-editing system for the treatment of PsA comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTNFRSF4. In some embodiments, the CRISPR gene-editing system for the treatment of psoriatic arthritis comprises one or more sgRNAs targeting exon 2 of cTNFRSF4. In some embodiments, the CRISPR gene-editing system for the treatment of psoriatic arthritis comprises one or more sgRNAs targeting exon 3 of cTNFRSF4. In some embodiments, the CRISPR gene-editing system for the treatment of psoriatic arthritis comprises one or more sgRNAs targeting exon 4 of cTNFRSF4. In some embodiments, the CRISPR gene-editing system for the treatment of psoriatic arthritis comprises one or more sgRNAs targeting exon 5 of cTNFRSF4. In some embodiments, the CRISPR gene-editing system for the treatment of psoriatic arthritis comprises one or more sgRNAs targeting exon 6 of cTNFRSF4. In some embodiments, the CRISPR gene-editing system for the treatment of psoriatic arthritis comprises one or more sgRNAs targeting exon 7 of cTNFRSF4. In some embodiments, the CRISPR gene-editing system for the treatment of PsA comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTNFRSF4. In some embodiments, the CRISPR gene-editing system for the treatment of psoriatic arthritis comprises one or more sgRNAs targeting exon 2 of eTNFRSF4. In some embodiments, the CRISPR gene-editing system for the treatment of psoriatic arthritis comprises one or more sgRNAs targeting exon 3 of eTNFRSF4. In some embodiments, the CRISPR gene-editing system for the treatment of psoriatic arthritis comprises one or more sgRNAs targeting exon 4 of eTNFRSF4. In some embodiments, the CRISPR gene-editing system for the treatment of psoriatic arthritis comprises one or more sgRNAs targeting exon 5 of eTNFRSF4. In some embodiments, the CRISPR gene-editing system for the treatment of psoriatic arthritis comprises one or more sgRNAs targeting exon 6 of eTNFRSF4. In some embodiments, the CRISPR gene-editing system for the treatment of psoriatic arthritis comprises one or more sgRNAs targeting exon 7 of eTNFRSF4. In some embodiments, the CRISPR gene-editing system for the treatment of PsA comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTNFRSF4. In some embodiments, the CRISPR gene-editing system for the treatment of psoriatic arthritis comprises one or more sgRNAs targeting exon 2 of fTNFRSF4. In some embodiments, the CRISPR gene-editing system for the treatment of psoriatic arthritis comprises one or more sgRNAs targeting exon 3 of fTNFRSF4. In some embodiments, the CRISPR gene-editing system for the treatment of psoriatic arthritis comprises one or more sgRNAs targeting exon 4 of fTNFRSF4. In some embodiments, the CRISPR gene-editing system for the treatment of psoriatic arthritis comprises one or more sgRNAs targeting exon 5 of fTNFRSF4. In some embodiments, the CRISPR gene-editing system for the treatment of psoriatic arthritis comprises one or more sgRNAs targeting exon 6 of fTNFRSF4. In some embodiments, the CRISPR gene-editing system for the treatment of psoriatic arthritis comprises one or more sgRNAs targeting exon 7 of fTNFRSF4. In some embodiments, the CRISPR gene-editing system for the treatment of PsA comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting TNFRSF11A. In some embodiments, the CRISPR gene-editing system for the treatment of psoriatic arthritis comprises one or more sgRNAs targeting hTNFRSF11A. In some embodiments, the CRISPR gene-editing system for the treatment of psoriatic arthritis comprises one or more sgRNAs targeting cTNFRSF11A. In some embodiments, the CRISPR gene-editing system for the treatment of psoriatic arthritis comprises one or more sgRNAs targeting eTNFRSF11A. In some embodiments, the CRISPR gene-editing system for the treatment of psoriatic arthritis comprises one or more sgRNAs targeting fTNFRSF11A. In some embodiments, the CRISPR gene-editing system for the treatment of PsA comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of hTNFRSF11A. In some embodiments, the CRISPR gene-editing system for the treatment of psoriatic arthritis comprises one or more sgRNAs targeting exon 2 of hTNFRSF11A. In some embodiments, the CRISPR gene-editing system for the treatment of psoriatic arthritis comprises one or more sgRNAs targeting exon 3 of hTNFRSF11A. In some embodiments, the CRISPR gene-editing system for the treatment of psoriatic arthritis comprises one or more sgRNAs targeting exon 4 of hTNFRSF11A. In some embodiments, the CRISPR gene-editing system for the treatment of psoriatic arthritis comprises one or more sgRNAs targeting exon 5 of hTNFRSF11A. In some embodiments, the CRISPR gene-editing system for the treatment of psoriatic arthritis comprises one or more sgRNAs targeting exon 6 of hTNFRSF11A. In some embodiments, the CRISPR gene-editing system for the treatment of psoriatic arthritis comprises one or more sgRNAs targeting exon 7 of hTNFRSF11A. In some embodiments, the CRISPR gene-editing system for the treatment of psoriatic arthritis comprises one or more sgRNAs targeting exon 8 of hTNFRSF11A. In some embodiments, the CRISPR gene-editing system for the treatment of psoriatic arthritis comprises one or more sgRNAs targeting exon 9 of hTNFRSF11A. In some embodiments, the CRISPR gene-editing system for the treatment of psoriatic arthritis comprises one or more sgRNAs targeting exon 10 of hTNFRSF11A. In some embodiments, the CRISPR gene-editing system for the treatment of psoriatic arthritis comprises one or more sgRNAs targeting exon 11 of hTNFRSF11A. In some embodiments, the CRISPR gene-editing system for the treatment of psoriatic arthritis comprises one or more sgRNAs targeting exon 12 of hTNFRSF11A. In some embodiments, the CRISPR gene-editing system for the treatment of PsA comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of cTNFRSF11A. In some embodiments, the CRISPR gene-editing system for the treatment of psoriatic arthritis comprises one or more sgRNAs targeting exon 2 of cTNFRSF11A. In some embodiments, the CRISPR gene-editing system for the treatment of psoriatic arthritis comprises one or more sgRNAs targeting exon 3 of cTNFRSF11A. In some embodiments, the CRISPR gene-editing system for the treatment of psoriatic arthritis comprises one or more sgRNAs targeting exon 4 of cTNFRSF11A. In some embodiments, the CRISPR gene-editing system for the treatment of psoriatic arthritis comprises one or more sgRNAs targeting exon 5 of cTNFRSF11A. In some embodiments, the CRISPR gene-editing system for the treatment of psoriatic arthritis comprises one or more sgRNAs targeting exon 6 of cTNFRSF11A. In some embodiments, the CRISPR gene-editing system for the treatment of psoriatic arthritis comprises one or more sgRNAs targeting exon 7 of cTNFRSF11A. In some embodiments, the CRISPR gene-editing system for the treatment of psoriatic arthritis comprises one or more sgRNAs targeting exon 8 of cTNFRSF11A. In some embodiments, the CRISPR gene-editing system for the treatment of psoriatic arthritis comprises one or more sgRNAs targeting exon 9 of cTNFRSF11A. In some embodiments, the CRISPR gene-editing system for the treatment of psoriatic arthritis comprises one or more sgRNAs targeting exon 10 of cTNFRSF11A. In some embodiments, the CRISPR gene-editing system for the treatment of psoriatic arthritis comprises one or more sgRNAs targeting exon 11 of cTNFRSF11A. In some embodiments, the CRISPR gene-editing system for the treatment of PsA comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of eTNFRSF11A. In some embodiments, the CRISPR gene-editing system for the treatment of psoriatic arthritis comprises one or more sgRNAs targeting exon 2 of eTNFRSF11A. In some embodiments, the CRISPR gene-editing system for the treatment of psoriatic arthritis comprises one or more sgRNAs targeting exon 3 of eTNFRSF11A. In some embodiments, the CRISPR gene-editing system for the treatment of psoriatic arthritis comprises one or more sgRNAs targeting exon 4 of eTNFRSF11A. In some embodiments, the CRISPR gene-editing system for the treatment of psoriatic arthritis comprises one or more sgRNAs targeting exon 5 of eTNFRSF11A. In some embodiments, the CRISPR gene-editing system for the treatment of psoriatic arthritis comprises one or more sgRNAs targeting exon 6 of eTNFRSF11A. In some embodiments, the CRISPR gene-editing system for the treatment of psoriatic arthritis comprises one or more sgRNAs targeting exon 7 of eTNFRSF11A. In some embodiments, the CRISPR gene-editing system for the treatment of psoriatic arthritis comprises one or more sgRNAs targeting exon 8 of eTNFRSF11A. In some embodiments, the CRISPR gene-editing system for the treatment of psoriatic arthritis comprises one or more sgRNAs targeting exon 9 of eTNFRSF11A. In some embodiments, the CRISPR gene-editing system for the treatment of psoriatic arthritis comprises one or more sgRNAs targeting exon 10 of eTNFRSF11A. In some embodiments, the CRISPR gene-editing system for the treatment of PsA comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of psoriatic arthritis comprises one or more sgRNAs targeting exon 1 of fTNFRSF11A. In some embodiments, the CRISPR gene-editing system for the treatment of psoriatic arthritis comprises one or more sgRNAs targeting exon 2 of fTNFRSF11A. In some embodiments, the CRISPR gene-editing system for the treatment of psoriatic arthritis comprises one or more sgRNAs targeting exon 3 of fTNFRSF11A. In some embodiments, the CRISPR gene-editing system for the treatment of psoriatic arthritis comprises one or more sgRNAs targeting exon 4 of fTNFRSF11A. In some embodiments, the CRISPR gene-editing system for the treatment of psoriatic arthritis comprises one or more sgRNAs targeting exon 5 of fTNFRSF11A. In some embodiments, the CRISPR gene-editing system for the treatment of psoriatic arthritis comprises one or more sgRNAs targeting exon 6 of fTNFRSF11A. In some embodiments, the CRISPR gene-editing system for the treatment of psoriatic arthritis comprises one or more sgRNAs targeting exon 7 of fTNFRSF11A. In some embodiments, the CRISPR gene-editing system for the treatment of psoriatic arthritis comprises one or more sgRNAs targeting exon 8 of fTNFRSF11A. In some embodiments, the CRISPR gene-editing system for the treatment of psoriatic arthritis comprises one or more sgRNAs targeting exon 9 of fTNFRSF11A. In some embodiments, the CRISPR gene-editing system for the treatment of psoriatic arthritis comprises one or more sgRNAs targeting exon 10 of fTNFRSF11A. In some embodiments, the CRISPR gene-editing system for the treatment of psoriatic arthritis comprises one or more sgRNAs targeting exon 11 of fTNFRSF11A. In some embodiments, the CRISPR gene-editing system for the treatment of PsA comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### D. Gout

In one aspect, the present disclosure encompasses treatments for gout and other crystallopathies affecting the joint, e.g., octacalcium phosphate and calcium pyrophosphate dihydrate in horses. In some embodiments, the gout treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the gout treatment comprises a CRISPR gene-editing system targeting hIL1R1. In some embodiments, the gout treatment comprises a CRISPR gene-editing system targeting cIL1R1. In some embodiments, the gout treatment comprises a CRISP gene-editing system R targeting eIL1R1. In some embodiments, the gout treatment comprises a CRISPR gene-editing system targeting fIL1R1. In some embodiments, the CRISPR gene-editing system for the treatment of gout comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hIL1R1. In some embodiments, the CRISPR gene-editing system for the treatment of gout comprises one or more sgRNAs targeting exon 2 of hIL1R1. In some embodiments, the CRISPR gene-editing system for the treatment of gout comprises one or more sgRNAs targeting exon 3 of hIL1R1. In some embodiments, the CRISPR gene-editing system for the treatment of gout comprises one or more sgRNAs targeting exon 4 of hIL1R1. In some embodiments, the CRISPR gene-editing system for the treatment of gout comprises one or more sgRNAs targeting exon 5 of hIL1R1. In some embodiments, the CRISPR gene-editing system for the treatment of gout comprises one or more sgRNAs targeting exon 6 of hIL1R1. In some embodiments, the CRISPR gene-editing system for the treatment of gout comprises one or more sgRNAs targeting exon 7 of hIL1R1. In some embodiments, the CRISPR gene-editing system for the treatment of gout comprises one or more sgRNAs targeting exon 8 of hIL1R1. In some embodiments, the CRISPR gene-editing system for the treatment of gout comprises one or more sgRNAs targeting exon 9 of hIL1R1. In some embodiments, the CRISPR gene-editing system for the treatment of gout comprises one or more sgRNAs targeting exon 10 of hIL1R1. In some embodiments, the CRISPR gene-editing system for the treatment of gout comprises one or more sgRNAs targeting exon 11 of hIL1R1. In some embodiments, the CRISPR gene-editing system for the treatment of gout comprises one or more sgRNAs targeting exon 12 of hIL1R1. In some embodiments, the CRISPR gene-editing system for the treatment of gout comprises one or more sgRNAs targeting exon 13 of hIL1R1. In some embodiments, the CRISPR gene-editing system for the treatment of gout comprises one or more sgRNAs targeting exon 14 of hIL1R1. In some embodiments, the CRISPR gene-editing system for the treatment of gout comprises one or more sgRNAs targeting exon 15 of hIL1R1. In some embodiments, the CRISPR gene-editing system for the treatment of gout comprises one or more sgRNAs targeting exon 16 of hIL1R1. In some embodiments, the CRISPR gene-editing system for the treatment of gout comprises one or more sgRNAs targeting exon 17 of hIL1R1. In some embodiments, the CRISPR gene-editing system for the treatment of gout comprises one or more sgRNAs targeting exon 18 of hIL1R1. In some embodiments, the CRISPR gene-editing system for the treatment of gout comprises one or more sgRNAs targeting exon 19 of hIL1R1. In some embodiments, the CRISPR gene-editing system for the treatment of gout comprises one or more sgRNAs targeting exon 20 of hIL1R1. In some embodiments, the CRISPR gene-editing system for the treatment of gout comprises one or more sgRNAs targeting exon 21 of hIL1R1. In some embodiments, the CRISPR gene-editing system for the treatment of gout comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cIL1R1. In some embodiments, the CRISPR gene-editing system for the treatment of gout comprises one or more sgRNAs targeting exon 2 of cIL1R1. In some embodiments, the CRISPR gene-editing system for the treatment of gout comprises one or more sgRNAs targeting exon 3 of cIL1R1. In some embodiments, the CRISPR gene-editing system for the treatment of gout comprises one or more sgRNAs targeting exon 4 of cIL1R1. In some embodiments, the CRISPR gene-editing system for the treatment of gout comprises one or more sgRNAs targeting exon 5 of cIL1R1. In some embodiments, the CRISPR gene-editing system for the treatment of gout comprises one or more sgRNAs targeting exon 6 of cIL1R1. In some embodiments, the CRISPR gene-editing system for the treatment of gout comprises one or more sgRNAs targeting exon 7 of cIL1R1. In some embodiments, the CRISPR gene-editing system for the treatment of gout comprises one or more sgRNAs targeting exon 8 of cIL1R1. In some embodiments, the CRISPR gene-editing system for the treatment of gout comprises one or more sgRNAs targeting exon 9 of cIL1R1. In some embodiments, the CRISPR gene-editing system for the treatment of gout comprises one or more sgRNAs targeting exon 10 of cIL1R1. In some embodiments, the CRISPR gene-editing system for the treatment of gout comprises one or more sgRNAs targeting exon 11 of cIL1R1. In some embodiments, the CRISPR gene-editing system for the treatment of gout comprises one or more sgRNAs targeting exon 12 of cIL1R1. In some embodiments, the CRISPR gene-editing system for the treatment of gout comprises one or more sgRNAs targeting exon 13 of cIL1R1. In some embodiments, the CRISPR gene-editing system for the treatment of gout comprises one or more sgRNAs targeting exon 14 of cIL1R1. In some embodiments, the CRISPR gene-editing system for the treatment of gout comprises one or more sgRNAs targeting exon 15 of cIL1R1. In some embodiments, the CRISPR gene-editing system for the treatment of gout comprises one or more sgRNAs targeting exon 16 of cIL1R1. In some embodiments, the CRISPR gene-editing system for the treatment of gout comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eIL1R1. In some embodiments, the CRISPR gene-editing system for the treatment of gout comprises one or more sgRNAs targeting exon 2 of eIL1R1. In some embodiments, the CRISPR gene-editing system for the treatment of gout comprises one or more sgRNAs targeting exon 3 of eIL1R1. In some embodiments, the CRISPR gene-editing system for the treatment of gout comprises one or more sgRNAs targeting exon 4 of eIL1R1. In some embodiments, the CRISPR gene-editing system for the treatment of gout comprises one or more sgRNAs targeting exon 5 of eIL1R1. In some embodiments, the CRISPR gene-editing system for the treatment of gout comprises one or more sgRNAs targeting exon 6 of eIL1R1. In some embodiments, the CRISPR gene-editing system for the treatment of gout comprises one or more sgRNAs targeting exon 7 of eIL1R1. In some embodiments, the CRISPR gene-editing system for the treatment of gout comprises one or more sgRNAs targeting exon 8 of eIL1R1. In some embodiments, the CRISPR gene-editing system for the treatment of gout comprises one or more sgRNAs targeting exon 9 of eIL1R. In some embodiments, the CRISPR gene-editing system for the treatment of gout comprises one or more sgRNAs targeting exon 10 of eIL1R1. In some embodiments, the CRISPR gene-editing system for the treatment of gout comprises one or more sgRNAs targeting exon 11 of eIL1R1. In some embodiments, the CRISPR gene-editing system for the treatment of gout comprises one or more sgRNAs targeting exon 12 of eIL1R1. In some embodiments, the CRISPR gene-editing system for the treatment of gout comprises one or more sgRNAs targeting exon 13 of eIL1R1. In some embodiments, the CRISPR gene-editing system for the treatment of gout comprises one or more sgRNAs targeting exon 14 of eIL1R1. In some embodiments, the CRISPR gene-editing system for the treatment of gout comprises one or more sgRNAs targeting exon 15 of fIL1R1. In some embodiments, the CRISPR gene-editing system for the treatment of gout comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fIL1R1. In some embodiments, the CRISPR gene-editing system for the treatment of gout comprises one or more sgRNAs targeting exon 2 of fIL1R1. In some embodiments, the CRISPR gene-editing system for the treatment of gout comprises one or more sgRNAs targeting exon 3 of fIL1R1. In some embodiments, the CRISPR gene-editing system for the treatment of gout comprises one or more sgRNAs targeting exon 4 of fIL1R1. In some embodiments, the CRISPR gene-editing system for the treatment of gout comprises one or more sgRNAs targeting exon 5 of fIL1R1. In some embodiments, the CRISPR gene-editing system for the treatment of gout comprises one or more sgRNAs targeting exon 6 of fIL1R1. In some embodiments, the CRISPR gene-editing system for the treatment of gout comprises one or more sgRNAs targeting exon 7 of fIL1R1. In some embodiments, the CRISPR gene-editing system for the treatment of gout comprises one or more sgRNAs targeting exon 8 of fIL1R1. In some embodiments, the CRISPR gene-editing system for the treatment of gout comprises one or more sgRNAs targeting exon 9 of fIL1R1. In some embodiments, the CRISPR gene-editing system for the treatment of gout comprises one or more sgRNAs targeting exon 10 of fIL1R1. In some embodiments, the CRISPR gene-editing system for the treatment of gout comprises one or more sgRNAs targeting exon 11 of fIL1R. In some embodiments, the CRISPR gene-editing system for the treatment of gout comprises one or more sgRNAs targeting exon 12 of fIL1R1. In some embodiments, the CRISPR gene-editing system for the treatment of gout comprises one or more sgRNAs targeting exon 13 of fIL1R1. In some embodiments, the CRISPR gene-editing system for the treatment of gout comprises one or more sgRNAs targeting exon 14 of fIL1R1. In some embodiments, the CRISPR gene-editing system for the treatment of gout comprises one or more sgRNAs targeting exon 15 of fIL1R. In some embodiments, the CRISPR gene-editing system for the treatment of gout comprises one or more sgRNAs targeting exon 16 of fIL1R1. In some embodiments, the CRISPR gene-editing system for the treatment of gout comprises one or more sgRNAs targeting exon 17 of fIL1R1. In some embodiments, the CRISPR gene-editing system for the treatment of gout comprises one or more sgRNAs targeting exon 18 of fIL1R1. In some embodiments, the CRISPR gene-editing system for the treatment of gout comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting IL1RAP. In some embodiments, the CRISPR gene-editing system for the treatment of gout comprises one or more sgRNAs targeting hIL1RAP. In some embodiments, the CRISPR gene-editing system for the treatment of gout comprises one or more sgRNAs targeting cIL1RAP. In some embodiments, the CRISPR gene-editing system for the treatment of gout comprises one or more sgRNAs targeting eIL1RAP. In some embodiments, the CRISPR gene-editing system for the treatment of gout comprises one or more sgRNAs targeting fIL1RAP. In some embodiments, the CRISPR gene-editing system for the treatment of gout comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hIL1RAP. In some embodiments, the CRISPR gene-editing system for the treatment of gout comprises one or more sgRNAs targeting exon 2 of hIL1RAP. In some embodiments, the CRISPR gene-editing system for the treatment of gout comprises one or more sgRNAs targeting exon 3 of hIL1RAP. In some embodiments, the CRISPR gene-editing system for the treatment of gout comprises one or more sgRNAs targeting exon 4 of hIL1RAP. In some embodiments, the CRISPR gene-editing system for the treatment of gout comprises one or more sgRNAs targeting exon 5 of hIL1RAP. In some embodiments, the CRISPR gene-editing system for the treatment of gout comprises one or more sgRNAs targeting exon 6 of hIL1RAP. In some embodiments, the CRISPR gene-editing system for the treatment of gout comprises one or more sgRNAs targeting exon 7 of hIL1RAP. In some embodiments, the CRISPR gene-editing system for the treatment of gout comprises one or more sgRNAs targeting exon 8 of hIL1RAP. In some embodiments, the CRISPR gene-editing system for the treatment of gout comprises one or more sgRNAs targeting exon 9 of hIL1RAP. In some embodiments, the CRISPR gene-editing system for the treatment of gout comprises one or more sgRNAs targeting exon 10 of hIL1RAP. In some embodiments, the CRISPR gene-editing system for the treatment of gout comprises one or more sgRNAs targeting exon 11 of hIL1RAP. In some embodiments, the CRISPR gene-editing system for the treatment of gout comprises one or more sgRNAs targeting exon 12 of hIL1RAP. In some embodiments, the CRISPR gene-editing system for the treatment of gout comprises one or more sgRNAs targeting exon 13 of hIL1RAP. In some embodiments, the CRISPR gene-editing system for the treatment of gout comprises one or more sgRNAs targeting exon 14 of hIL1RAP. In some embodiments, the CRISPR gene-editing system for the treatment of gout comprises one or more sgRNAs targeting exon 15 of hIL1RAP. In some embodiments, the CRISPR gene-editing system for the treatment of gout comprises one or more sgRNAs targeting exon 16 of hIL1RAP. In some embodiments, the CRISPR gene-editing system for the treatment of gout comprises one or more sgRNAs targeting exon 17 of hIL1RAP. In some embodiments, the CRISPR gene-editing system for the treatment of gout comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cIL1RAP. In some embodiments, the CRISPR gene-editing system for the treatment of gout comprises one or more sgRNAs targeting exon 2 of cIL1RAP. In some embodiments, the CRISPR gene-editing system for the treatment of gout comprises one or more sgRNAs targeting exon 3 of cIL1RAP. In some embodiments, the CRISPR gene-editing system for the treatment of gout comprises one or more sgRNAs targeting exon 4 of cIL1RAP. In some embodiments, the CRISPR gene-editing system for the treatment of gout comprises one or more sgRNAs targeting exon 5 of cIL1RAP. In some embodiments, the CRISPR gene-editing system for the treatment of gout comprises one or more sgRNAs targeting exon 6 of cIL1RAP. In some embodiments, the CRISPR gene-editing system for the treatment of gout comprises one or more sgRNAs targeting exon 7 of cIL1RAP. In some embodiments, the CRISPR gene-editing system for the treatment of gout comprises one or more sgRNAs targeting exon 8 of cIL1RAP. In some embodiments, the CRISPR gene-editing system for the treatment of gout comprises one or more sgRNAs targeting exon 9 of cIL1RAP. In some embodiments, the CRISPR gene-editing system for the treatment of gout comprises one or more sgRNAs targeting exon 10 of cIL1RAP. In some embodiments, the CRISPR gene-editing system for the treatment of gout comprises one or more sgRNAs targeting exon 11 of cIL1RAP. In some embodiments, the CRISPR gene-editing system for the treatment of gout comprises one or more sgRNAs targeting exon 12 of cIL1RAP. In some embodiments, the CRISPR gene-editing system for the treatment of gout comprises one or more sgRNAs targeting exon 13 of cIL1RAP. In some embodiments, the CRISPR gene-editing system for the treatment of gout comprises one or more sgRNAs targeting exon 14 of cIL1RAP. In some embodiments, the CRISPR gene-editing system for the treatment of gout comprises one or more sgRNAs targeting exon 15 of cIL1RAP. In some embodiments, the CRISPR gene-editing system for the treatment of gout comprises one or more sgRNAs targeting exon 16 of cIL1RAP. In some embodiments, the CRISPR gene-editing system for the treatment of gout comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eIL1RAP. In some embodiments, the CRISPR gene-editing system for the treatment of gout comprises one or more sgRNAs targeting exon 2 of eIL1RAP. In some embodiments, the CRISPR gene-editing system for the treatment of gout comprises one or more sgRNAs targeting exon 3 of eIL1RAP. In some embodiments, the CRISPR gene-editing system for the treatment of gout comprises one or more sgRNAs targeting exon 4 of eIL1RAP. In some embodiments, the CRISPR gene-editing system for the treatment of gout comprises one or more sgRNAs targeting exon 5 of eIL1RAP. In some embodiments, the CRISPR gene-editing system for the treatment of gout comprises one or more sgRNAs targeting exon 6 of eIL1RAP. In some embodiments, the CRISPR gene-editing system for the treatment of gout comprises one or more sgRNAs targeting exon 7 of eIL1RAP. In some embodiments, the CRISPR gene-editing system for the treatment of gout comprises one or more sgRNAs targeting exon 8 of eIL1RAP. In some embodiments, the CRISPR gene-editing system for the treatment of gout comprises one or more sgRNAs targeting exon 9 of eIL1RAP. In some embodiments, the CRISPR gene-editing system for the treatment of gout comprises one or more sgRNAs targeting exon 10 of eIL1RAP. In some embodiments, the CRISPR gene-editing system for the treatment of gout comprises one or more sgRNAs targeting exon 11 of eIL1RAP. In some embodiments, the CRISPR gene-editing system for the treatment of gout comprises one or more sgRNAs targeting exon 12 of eIL1RAP. In some embodiments, the CRISPR gene-editing system for the treatment of gout comprises one or more sgRNAs targeting exon 13 of eIL1RAP. In some embodiments, the CRISPR gene-editing system for the treatment of gout comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fIL1RAP. In some embodiments, the CRISPR gene-editing system for the treatment of gout comprises one or more sgRNAs targeting exon 2 of fIL1RAP. In some embodiments, the CRISPR gene-editing system for the treatment of gout comprises one or more sgRNAs targeting exon 3 of fIL1RAP. In some embodiments, the CRISPR gene-editing system for the treatment of gout comprises one or more sgRNAs targeting exon 4 of fIL1RAP. In some embodiments, the CRISPR gene-editing system for the treatment of gout comprises one or more sgRNAs targeting exon 5 of fIL1RAP. In some embodiments, the CRISPR gene-editing system for the treatment of gout comprises one or more sgRNAs targeting exon 6 of fIL1RAP. In some embodiments, the CRISPR gene-editing system for the treatment of gout comprises one or more sgRNAs targeting exon 7 of fIL1RAP. In some embodiments, the CRISPR gene-editing system for the treatment of gout comprises one or more sgRNAs targeting exon 8 of fIL1RAP. In some embodiments, the CRISPR gene-editing system for the treatment of gout comprises one or more sgRNAs targeting exon 9 of fIL1RAP. In some embodiments, the CRISPR gene-editing system for the treatment of gout comprises one or more sgRNAs targeting exon 10 of fIL1RAP. In some embodiments, the CRISPR gene-editing system for the treatment of gout comprises one or more sgRNAs targeting exon 11 of fIL1RAP. In some embodiments, the CRISPR gene-editing system for the treatment of gout comprises one or more sgRNAs targeting exon 12 of fIL1RAP. In some embodiments, the CRISPR gene-editing system for the treatment of gout comprises one or more sgRNAs targeting exon 13 of fIL1RAP. In some embodiments, the CRISPR gene-editing system for the treatment of gout comprises one or more sgRNAs targeting exon 14 of fIL1RAP. In some embodiments, the CRISPR gene-editing system for the treatment of gout comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TGFBR1. In some embodiments, the CRISPR gene-editing system for the treatment of gout comprises one or more sgRNAs targeting hTGFBR1. In some embodiments, the CRISPR gene-editing system for the treatment of gout comprises one or more sgRNAs targeting cTGFBR1. In some embodiments, the CRISPR gene-editing system for the treatment of gout comprises one or more sgRNAs targeting e TGFBR1. In some embodiments, the CRISPR gene-editing system for the treatment of gout comprises one or more sgRNAs targeting fTGFBR1. In some embodiments, the CRISPR gene-editing system for the treatment of gout comprising one or more sgRNAs targeting an exon of TRGBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTGFBR1. In some embodiments, the CRISPR gene-editing system for the treatment of gout comprises one or more sgRNAs targeting exon 2 of hTGFBR1. In some embodiments, the CRISPR gene-editing system for the treatment of gout comprises one or more sgRNAs targeting exon 3 of hTGFBR1. In some embodiments, the CRISPR gene-editing system for the treatment of gout comprises one or more sgRNAs targeting exon 4 of hTGFBR1. In some embodiments, the CRISPR gene-editing system for the treatment of gout comprises one or more sgRNAs targeting exon 5 of hTGFBR1. In some embodiments, the CRISPR gene-editing system for the treatment of gout comprises one or more sgRNAs targeting exon 6 of hTGFBR1. In some embodiments, the CRISPR gene-editing system for the treatment of gout comprises one or more sgRNAs targeting exon 7 of hTGFBR1. In some embodiments, the CRISPR gene-editing system for the treatment of gout comprises one or more sgRNAs targeting exon 8 of hTGFBR1. In some embodiments, the CRISPR gene-editing system for the treatment of gout comprises one or more sgRNAs targeting exon 9 of hTGFBR1. In some embodiments, the CRISPR gene-editing system for the treatment of gout comprises one or more sgRNAs targeting exon 10 of hTGFBR1. In some embodiments, the CRISPR gene-editing system for the treatment of gout comprises one or more sgRNAs targeting exon 11 of hTGFBR1. In some embodiments, the CRISPR gene-editing system for the treatment of gout comprising one or more sgRNAs targeting an exon of hTRGBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTGFBR1. In some embodiments, the CRISPR gene-editing system for the treatment of gout comprises one or more sgRNAs targeting exon 2 of cTGFBR1. In some embodiments, the CRISPR gene-editing system for the treatment of gout comprises one or more sgRNAs targeting exon 3 of cTGFBR1. In some embodiments, the CRISPR gene-editing system for the treatment of gout comprises one or more sgRNAs targeting exon 4 of cTGFBR1. In some embodiments, the CRISPR gene-editing system for the treatment of gout comprises one or more sgRNAs targeting exon 5 of cTGFBR1. In some embodiments, the CRISPR gene-editing system for the treatment of gout comprises one or more sgRNAs targeting exon 6 of cTGFBR1. In some embodiments, the CRISPR gene-editing system for the treatment of gout comprises one or more sgRNAs targeting exon 7 of cTGFBR1. In some embodiments, the CRISPR gene-editing system for the treatment of gout comprises one or more sgRNAs targeting exon 8 of cTGFBR1. In some embodiments, the CRISPR gene-editing system for the treatment of gout comprises one or more sgRNAs targeting exon 9 of cTGFBR1. In some embodiments, the CRISPR gene-editing system for the treatment of gout comprising one or more sgRNAs targeting an exon of cTRGBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTGFBR1. In some embodiments, the CRISPR gene-editing system for the treatment of gout comprises one or more sgRNAs targeting exon 2 of eTGFBR1. In some embodiments, the CRISPR gene-editing system for the treatment of gout comprises one or more sgRNAs targeting exon 3 of eTGFBR1. In some embodiments, the CRISPR gene-editing system for the treatment of gout comprises one or more sgRNAs targeting exon 4 of eTGFBR1. In some embodiments, the CRISPR gene-editing system for the treatment of gout comprises one or more sgRNAs targeting exon 5 of eTGFBR1. In some embodiments, the CRISPR gene-editing system for the treatment of gout comprises one or more sgRNAs targeting exon 6 of eTGFBR1. In some embodiments, the CRISPR gene-editing system for the treatment of gout comprises one or more sgRNAs targeting exon 7 of eTGFBR1. In some embodiments, the CRISPR gene-editing system for the treatment of gout comprises one or more sgRNAs targeting exon 8 of eTGFBR1. In some embodiments, the CRISPR gene-editing system for the treatment of gout comprises one or more sgRNAs targeting exon 9 of eTGFBR1. In some embodiments, the CRISPR gene-editing system for the treatment of gout comprises one or more sgRNAs targeting exon 10 of eTGFBR1. In some embodiments, the CRISPR gene-editing system for the treatment of gout comprises one or more sgRNAs targeting exon 11 of eTGFBR1. In some embodiments, the CRISPR gene-editing system for the treatment of gout comprises one or more sgRNAs targeting exon 12 of eTGFBR1. In some embodiments, the CRISPR gene-editing system for the treatment of gout comprising one or more sgRNAs targeting an exon of eTRGBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTGFBR1. In some embodiments, the CRISPR gene-editing system for the treatment of gout comprises one or more sgRNAs targeting exon 2 of fTGFBR1. In some embodiments, the CRISPR gene-editing system for the treatment of gout comprises one or more sgRNAs targeting exon 3 of fTGFBR1. In some embodiments, the CRISPR gene-editing system for the treatment of gout comprises one or more sgRNAs targeting exon 4 of fTGFBR1. In some embodiments, the CRISPR gene-editing system for the treatment of gout comprises one or more sgRNAs targeting exon 5 of fTGFBR1. In some embodiments, the CRISPR gene-editing system for the treatment of gout comprises one or more sgRNAs targeting exon 6 of fTGFBR1. In some embodiments, the CRISPR gene-editing system for the treatment of gout comprises one or more sgRNAs targeting exon 7 of fTGFBR1. In some embodiments, the CRISPR gene-editing system for the treatment of gout comprises one or more sgRNAs targeting exon 8 of fTGFBR1. In some embodiments, the CRISPR gene-editing system for the treatment of gout comprises one or more sgRNAs targeting exon 9 of fTGFBR1. In some embodiments, the CRISPR gene-editing system for the treatment of gout comprises one or more sgRNAs targeting exon 10 of fTGFBR1. In some embodiments, the CRISPR gene-editing system for the treatment of gout comprises one or more sgRNAs targeting exon 11 of fTGFBR1. In some embodiments, the CRISPR gene-editing system for the treatment of gout comprising one or more sgRNAs targeting an exon of fTRGBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TGFBR2. In some embodiments, the CRISPR gene-editing system for the treatment of gout comprises one or more sgRNAs targeting hTGFBR2. In some embodiments, the CRISPR gene-editing system for the treatment of gout comprises one or more sgRNAs targeting cTGFBR2. In some embodiments, the CRISPR gene-editing system for the treatment of gout comprises one or more sgRNAs targeting eTGFBR2. In some embodiments, the CRISPR gene-editing system for the treatment of gout comprises one or more sgRNAs targeting fTGFBR2. In some embodiments, the CRISPR gene-editing system for the treatment of gout comprising one or more sgRNAs targeting an exon of TRGBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTGFBR2. In some embodiments, the CRISPR gene-editing system for the treatment of gout comprises one or more sgRNAs targeting exon 2 of hTGFBR2. In some embodiments, the CRISPR gene-editing system for the treatment of gout comprises one or more sgRNAs targeting exon 3 of hTGFBR2. In some embodiments, the CRISPR gene-editing system for the treatment of gout comprises one or more sgRNAs targeting exon 4 of hTGFBR2. In some embodiments, the CRISPR gene-editing system for the treatment of gout comprises one or more sgRNAs targeting exon 5 of hTGFBR2. In some embodiments, the CRISPR gene-editing system for the treatment of gout comprises one or more sgRNAs targeting exon 6 of hTGFBR2. In some embodiments, the CRISPR gene-editing system for the treatment of gout comprises one or more sgRNAs targeting exon 7 of hTGFBR2. In some embodiments, the CRISPR gene-editing system for the treatment of gout comprises one or more sgRNAs targeting exon 8 of hTGFBR2. In some embodiments, the CRISPR gene-editing system for the treatment of gout comprises one or more sgRNAs targeting exon 9 of hTGFBR2. In some embodiments, the CRISPR gene-editing system for the treatment of gout comprising one or more sgRNAs targeting an exon of hTRGBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTGFBR2. In some embodiments, the CRISPR gene-editing system for the treatment of gout comprises one or more sgRNAs targeting exon 2 of cTGFBR2. In some embodiments, the CRISPR gene-editing system for the treatment of gout comprises one or more sgRNAs targeting exon 3 of cTGFBR2. In some embodiments, the CRISPR gene-editing system for the treatment of gout comprises one or more sgRNAs targeting exon 4 of cTGFBR2. In some embodiments, the CRISPR gene-editing system for the treatment of gout comprises one or more sgRNAs targeting exon 5 of cTGFBR2. In some embodiments, the CRISPR gene-editing system for the treatment of gout comprises one or more sgRNAs targeting exon 6 of cTGFBR2. In some embodiments, the CRISPR gene-editing system for the treatment of gout comprises one or more sgRNAs targeting exon 7 of cTGFBR2. In some embodiments, the CRISPR gene-editing system for the treatment of gout comprises one or more sgRNAs targeting exon 8 of cTGFBR2. In some embodiments, the CRISPR gene-editing system for the treatment of gout comprising one or more sgRNAs targeting an exon of cTRGBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTGFBR2. In some embodiments, the CRISPR gene-editing system for the treatment of gout comprises one or more sgRNAs targeting exon 2 of eTGFBR2. In some embodiments, the CRISPR gene-editing system for the treatment of gout comprises one or more sgRNAs targeting exon 3 of eTGFBR2. In some embodiments, the CRISPR gene-editing system for the treatment of gout comprises one or more sgRNAs targeting exon 4 of eTGFBR2. In some embodiments, the CRISPR gene-editing system for the treatment of gout comprises one or more sgRNAs targeting exon 5 of eTGFBR2. In some embodiments, the CRISPR gene-editing system for the treatment of gout comprises one or more sgRNAs targeting exon 6 of eTGFBR2. In some embodiments, the CRISPR gene-editing system for the treatment of gout comprises one or more sgRNAs targeting exon 7 of eTGFBR2. In some embodiments, the CRISPR gene-editing system for the treatment of gout comprises one or more sgRNAs targeting exon 8 of eTGFBR2. In some embodiments, the CRISPR gene-editing system for the treatment of gout comprises one or more sgRNAs targeting exon 9 of eTGFBR2. In some embodiments, the CRISPR gene-editing system for the treatment of gout comprises one or more sgRNAs targeting exon 10 of eTGFBR2. In some embodiments, the CRISPR gene-editing system for the treatment of gout comprising one or more sgRNAs targeting an exon of eTRGBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTGFBR2. In some embodiments, the CRISPR gene-editing system for the treatment of gout comprises one or more sgRNAs targeting exon 2 of fTGFBR2. In some embodiments, the CRISPR gene-editing system for the treatment of gout comprises one or more sgRNAs targeting exon 3 of fTGFBR2. In some embodiments, the CRISPR gene-editing system for the treatment of gout comprises one or more sgRNAs targeting exon 4 of fTGFBR2. In some embodiments, the CRISPR gene-editing system for the treatment of gout comprises one or more sgRNAs targeting exon 5 of fTGFBR2. In some embodiments, the CRISPR gene-editing system for the treatment of gout comprises one or more sgRNAs targeting exon 6 of fTGFBR2. In some embodiments, the CRISPR gene-editing system for the treatment of gout comprises one or more sgRNAs targeting exon 7 of fTGFBR2. In some embodiments, the CRISPR gene-editing system for the treatment of gout comprises one or more sgRNAs targeting exon 8 of fTGFBR2. In some embodiments, the CRISPR gene-editing system for the treatment of gout comprising one or more sgRNAs targeting an exon of fTRGBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting IL6R. In some embodiments, the CRISPR gene-editing system for the treatment of gout comprises one or more sgRNAs targeting hIL6R. In some embodiments, the CRISPR gene-editing system for the treatment of gout comprises one or more sgRNAs targeting cIL6R. In some embodiments, the CRISPR gene-editing system for the treatment of gout comprises one or more sgRNAs targeting eIL6R. In some embodiments, the CRISPR gene-editing system for the treatment of gout comprises one or more sgRNAs targeting fIL6R. In some embodiments, the CRISPR gene-editing system for the treatment of gout comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hIL6R. In some embodiments, the CRISPR gene-editing system for the treatment of gout comprises one or more sgRNAs targeting exon 2 of hIL6R. In some embodiments, the CRISPR gene-editing system for the treatment of gout comprises one or more sgRNAs targeting exon 3 of hIL6R. In some embodiments, the CRISPR gene-editing system for the treatment of gout comprises one or more sgRNAs targeting exon 4 of hIL6R. In some embodiments, the CRISPR gene-editing system for the treatment of gout comprises one or more sgRNAs targeting exon 5 of hIL6R. In some embodiments, the CRISPR gene-editing system for the treatment of gout comprises one or more sgRNAs targeting exon 6 of hIL6R. In some embodiments, the CRISPR gene-editing system for the treatment of gout comprises one or more sgRNAs targeting exon 7 of hIL6R. In some embodiments, the CRISPR gene-editing system for the treatment of gout comprises one or more sgRNAs targeting exon 8 of hIL6R. In some embodiments, the CRISPR gene-editing system for the treatment of gout comprises one or more sgRNAs targeting exon 9 of hIL6R. In some embodiments, the CRISPR gene-editing system for the treatment of gout comprises one or more sgRNAs targeting exon 10 of hIL6R. In some embodiments, the CRISPR gene-editing system for the treatment of gout comprises one or more sgRNAs targeting exon 11 of hIL6R In some embodiments, the CRISPR gene-editing system for the treatment of gout comprises one or more sgRNAs targeting exon 12 of hIL6R. In some embodiments, the CRISPR gene-editing system for the treatment of gout comprises one or more sgRNAs targeting exon 13 of hIL6R. In some embodiments, the CRISPR gene-editing system for the treatment of gout comprises one or more sgRNAs targeting exon 14 of hIL6R. In some embodiments, the CRISPR gene-editing system for the treatment of gout comprises one or more sgRNAs targeting exon 15 of hIL6R. In some embodiments, the CRISPR gene-editing system for the treatment of gout comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cIL6R. In some embodiments, the CRISPR gene-editing system for the treatment of gout comprises one or more sgRNAs targeting exon 2 of cIL6R. In some embodiments, the CRISPR gene-editing system for the treatment of gout comprises one or more sgRNAs targeting exon 3 of cIL6R. In some embodiments, the CRISPR gene-editing system for the treatment of gout comprises one or more sgRNAs targeting exon 4 of cIL6R. In some embodiments, the CRISPR gene-editing system for the treatment of gout comprises one or more sgRNAs targeting exon 5 of cIL6R. In some embodiments, the CRISPR gene-editing system for the treatment of gout comprises one or more sgRNAs targeting exon 6 of cIL6R. In some embodiments, the CRISPR gene-editing system for the treatment of gout comprises one or more sgRNAs targeting exon 7 of cIL6R. In some embodiments, the CRISPR gene-editing system for the treatment of gout comprises one or more sgRNAs targeting exon 8 of cIL6R. In some embodiments, the CRISPR gene-editing system for the treatment of gout comprises one or more sgRNAs targeting exon 9 of cIL6R. In some embodiments, the CRISPR gene-editing system for the treatment of gout comprises one or more sgRNAs targeting exon 10 of cIL6R. In some embodiments, the CRISPR gene-editing system for the treatment of gout comprises one or more sgRNAs targeting exon 11 of cIL6R. In some embodiments, the CRISPR gene-editing system for the treatment of gout comprises one or more sgRNAs targeting exon 12 of cIL6R. In some embodiments, the CRISPR gene-editing system for the treatment of gout comprises one or more sgRNAs targeting exon 13 of cIL6R. In some embodiments, the CRISPR gene-editing system for the treatment of gout comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eIL6R. In some embodiments, the CRISPR gene-editing system for the treatment of gout comprises one or more sgRNAs targeting exon 2 of eIL6R. In some embodiments, the CRISPR gene-editing system for the treatment of gout comprises one or more sgRNAs targeting exon 3 of eIL6R. In some embodiments, the CRISPR gene-editing system for the treatment of gout comprises one or more sgRNAs targeting exon 4 of eIL6R. In some embodiments, the CRISPR gene-editing system for the treatment of gout comprises one or more sgRNAs targeting exon 5 of eIL6R. In some embodiments, the CRISPR gene-editing system for the treatment of gout comprises one or more sgRNAs targeting exon 6 of eIL6R. In some embodiments, the CRISPR gene-editing system for the treatment of gout comprises one or more sgRNAs targeting exon 7 of eIL6R. In some embodiments, the CRISPR gene-editing system for the treatment of gout comprises one or more sgRNAs targeting exon 8 of eIL6R. In some embodiments, the CRISPR gene-editing system for the treatment of gout comprises one or more sgRNAs targeting exon 9 of eIL6R. In some embodiments, the CRISPR gene-editing system for the treatment of gout comprises one or more sgRNAs targeting exon 10 of eIL6R. In some embodiments, the CRISPR gene-editing system for the treatment of gout comprises one or more sgRNAs targeting exon 11 of eIL6R. In some embodiments, the CRISPR gene-editing system for the treatment of gout comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fIL6R. In some embodiments, the CRISPR gene-editing system for the treatment of gout comprises one or more sgRNAs targeting exon 2 of fIL6R. In some embodiments, the CRISPR gene-editing system for the treatment of gout comprises one or more sgRNAs targeting exon 3 of fIL6R. In some embodiments, the CRISPR gene-editing system for the treatment of gout comprises one or more sgRNAs targeting exon 4 of fIL6R. In some embodiments, the CRISPR gene-editing system for the treatment of gout comprises one or more sgRNAs targeting exon 5 of fIL6R. In some embodiments, the CRISPR gene-editing system for the treatment of gout comprises one or more sgRNAs targeting exon 6 of fIL6R. In some embodiments, the CRISPR gene-editing system for the treatment of gout comprises one or more sgRNAs targeting exon 7 of fIL6R. In some embodiments, the CRISPR gene-editing system for the treatment of gout comprises one or more sgRNAs targeting exon 8 of fIL6R. In some embodiments, the CRISPR gene-editing system for the treatment of gout comprises one or more sgRNAs targeting exon 9 of fIL6R. In some embodiments, the CRISPR gene-editing system for the treatment of gout comprises one or more sgRNAs targeting exon 10 of fIL6R. In some embodiments, the CRISPR gene-editing system for the treatment of gout comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting IL6ST. In some embodiments, the CRISPR gene-editing system for the treatment of gout comprises one or more sgRNAs targeting hIL6ST. In some embodiments, the CRISPR gene-editing system for the treatment of gout comprises one or more sgRNAs targeting cIL6ST. In some embodiments, the CRISPR gene-editing system for the treatment of gout comprises one or more sgRNAs targeting eIL6ST. In some embodiments, the CRISPR gene-editing system for the treatment of gout comprises one or more sgRNAs targeting fIL6ST. In some embodiments, the CRISPR gene-editing system for the treatment of gout comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hIL6ST. In some embodiments, the CRISPR gene-editing system for the treatment of gout comprises one or more sgRNAs targeting exon 2 of hIL6ST. In some embodiments, the CRISPR gene-editing system for the treatment of gout comprises one or more sgRNAs targeting exon 3 of hIL6ST. In some embodiments, the CRISPR gene-editing system for the treatment of gout comprises one or more sgRNAs targeting exon 4 of hIL6ST. In some embodiments, the CRISPR gene-editing system for the treatment of gout comprises one or more sgRNAs targeting exon 5 of hIL6ST. In some embodiments, the CRISPR gene-editing system for the treatment of gout comprises one or more sgRNAs targeting exon 6 of hIL6ST. In some embodiments, the CRISPR gene-editing system for the treatment of gout comprises one or more sgRNAs targeting exon 7 of hIL6ST. In some embodiments, the CRISPR gene-editing system for the treatment of gout comprises one or more sgRNAs targeting exon 8 of hIL6ST. In some embodiments, the CRISPR gene-editing system for the treatment of gout comprises one or more sgRNAs targeting exon 9 of hIL6ST. In some embodiments, the CRISPR gene-editing system for the treatment of gout comprises one or more sgRNAs targeting exon 10 of hIL6ST. In some embodiments, the CRISPR gene-editing system for the treatment of gout comprises one or more sgRNAs targeting exon 11 of hIL6ST. In some embodiments, the CRISPR gene-editing system for the treatment of gout comprises one or more sgRNAs targeting exon 12 of hIL6ST. In some embodiments, the CRISPR gene-editing system for the treatment of gout comprises one or more sgRNAs targeting exon 13 of hIL6ST. In some embodiments, the CRISPR gene-editing system for the treatment of gout comprises one or more sgRNAs targeting exon 14 of hIL6ST. In some embodiments, the CRISPR gene-editing system for the treatment of gout comprises one or more sgRNAs targeting exon 15 of hIL6ST. In some embodiments, the CRISPR gene-editing system for the treatment of gout comprises one or more sgRNAs targeting exon 16 of hIL6ST. In some embodiments, the CRISPR gene-editing system for the treatment of gout comprises one or more sgRNAs targeting exon 17 of hIL6ST. In some embodiments, the CRISPR gene-editing system for the treatment of gout comprises one or more sgRNAs targeting exon 18 of hIL6ST. In some embodiments, the CRISPR gene-editing system for the treatment of gout comprises one or more sgRNAs targeting exon 19 of hIL6ST. In some embodiments, the CRISPR gene-editing system for the treatment of gout comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cIL6ST. In some embodiments, the CRISPR gene-editing system for the treatment of gout comprises one or more sgRNAs targeting exon 2 of cIL6ST. In some embodiments, the CRISPR gene-editing system for the treatment of gout comprises one or more sgRNAs targeting exon 3 of cIL6ST. In some embodiments, the CRISPR gene-editing system for the treatment of gout comprises one or more sgRNAs targeting exon 4 of cIL6ST. In some embodiments, the CRISPR gene-editing system for the treatment of gout comprises one or more sgRNAs targeting exon 5 of cIL6ST. In some embodiments, the CRISPR gene-editing system for the treatment of gout comprises one or more sgRNAs targeting exon 6 of cIL6ST. In some embodiments, the CRISPR gene-editing system for the treatment of gout comprises one or more sgRNAs targeting exon 7 of cIL6ST. In some embodiments, the CRISPR gene-editing system for the treatment of gout comprises one or more sgRNAs targeting exon 8 of cIL6ST. In some embodiments, the CRISPR gene-editing system for the treatment of gout comprises one or more sgRNAs targeting exon 9 of cIL6ST. In some embodiments, the CRISPR gene-editing system for the treatment of gout comprises one or more sgRNAs targeting exon 10 of cIL6ST. In some embodiments, the CRISPR gene-editing system for the treatment of gout comprises one or more sgRNAs targeting exon 11 of cIL6ST In some embodiments, the CRISPR gene-editing system for the treatment of gout comprises one or more sgRNAs targeting exon 12 of cIL6ST. In some embodiments, the CRISPR gene-editing system for the treatment of gout comprises one or more sgRNAs targeting exon 13 of cIL6ST. In some embodiments, the CRISPR gene-editing system for the treatment of gout comprises one or more sgRNAs targeting exon 14 of cIL6ST. In some embodiments, the CRISPR gene-editing system for the treatment of gout comprises one or more sgRNAs targeting exon 15 of cIL6ST. In some embodiments, the CRISPR gene-editing system for the treatment of gout comprises one or more sgRNAs targeting exon 16 of cIL6ST. In some embodiments, the CRISPR gene-editing system for the treatment of gout comprises one or more sgRNAs targeting exon 17 of cIL6ST. In some embodiments, the CRISPR gene-editing system for the treatment of gout comprises one or more sgRNAs targeting exon 18 of cIL6ST. In some embodiments, the CRISPR gene-editing system for the treatment of gout comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eIL6ST. In some embodiments, the CRISPR gene-editing system for the treatment of gout comprises one or more sgRNAs targeting exon 2 of eIL6ST. In some embodiments, the CRISPR gene-editing system for the treatment of gout comprises one or more sgRNAs targeting exon 3 of eIL6ST. In some embodiments, the CRISPR gene-editing system for the treatment of gout comprises one or more sgRNAs targeting exon 4 of eIL6ST. In some embodiments, the CRISPR gene-editing system for the treatment of gout comprises one or more sgRNAs targeting exon 5 of eIL6ST. In some embodiments, the CRISPR gene-editing system for the treatment of gout comprises one or more sgRNAs targeting exon 6 of eIL6ST. In some embodiments, the CRISPR gene-editing system for the treatment of gout comprises one or more sgRNAs targeting exon 7 of eIL6ST. In some embodiments, the CRISPR gene-editing system for the treatment of gout comprises one or more sgRNAs targeting exon 8 of eIL6ST. In some embodiments, the CRISPR gene-editing system for the treatment of gout comprises one or more sgRNAs targeting exon 9 of eIL6ST. In some embodiments, the CRISPR gene-editing system for the treatment of gout comprises one or more sgRNAs targeting exon 10 of eIL6ST. In some embodiments, the CRISPR gene-editing system for the treatment of gout comprises one or more sgRNAs targeting exon 11 of eIL6ST In some embodiments, the CRISPR gene-editing system for the treatment of gout comprises one or more sgRNAs targeting exon 12 of eIL6ST. In some embodiments, the CRISPR gene-editing system for the treatment of gout comprises one or more sgRNAs targeting exon 13 of eIL6ST. In some embodiments, the CRISPR gene-editing system for the treatment of gout comprises one or more sgRNAs targeting exon 14 of eIL6ST. In some embodiments, the CRISPR gene-editing system for the treatment of gout comprises one or more sgRNAs targeting exon 15 of eIL6ST. In some embodiments, the CRISPR gene-editing system for the treatment of gout comprises one or more sgRNAs targeting exon 16 of eIL6ST. In some embodiments, the CRISPR gene-editing system for the treatment of gout comprises one or more sgRNAs targeting exon 17 of eIL6ST. In some embodiments, the CRISPR gene-editing system for the treatment of gout comprises one or more sgRNAs targeting exon 18 of eIL6ST. In some embodiments, the CRISPR gene-editing system for the treatment of gout comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fIL6ST. In some embodiments, the CRISPR gene-editing system for the treatment of gout comprises one or more sgRNAs targeting exon 2 of fIL6ST. In some embodiments, the CRISPR gene-editing system for the treatment of gout comprises one or more sgRNAs targeting exon 3 of fIL6ST. In some embodiments, the CRISPR gene-editing system for the treatment of gout comprises one or more sgRNAs targeting exon 4 of fIL6ST. In some embodiments, the CRISPR gene-editing system for the treatment of gout comprises one or more sgRNAs targeting exon 5 of fIL6ST. In some embodiments, the CRISPR gene-editing system for the treatment of gout comprises one or more sgRNAs targeting exon 6 of fIL6ST. In some embodiments, the CRISPR gene-editing system for the treatment of gout comprises one or more sgRNAs targeting exon 7 of fIL6ST. In some embodiments, the CRISPR gene-editing system for the treatment of gout comprises one or more sgRNAs targeting exon 8 of fIL6ST. In some embodiments, the CRISPR gene-editing system for the treatment of gout comprises one or more sgRNAs targeting exon 9 of fIL6ST. In some embodiments, the CRISPR gene-editing system for the treatment of gout comprises one or more sgRNAs targeting exon 10 of fIL6ST. In some embodiments, the CRISPR gene-editing system for the treatment of gout comprises one or more sgRNAs targeting exon 11 of fIL6ST In some embodiments, the CRISPR gene-editing system for the treatment of gout comprises one or more sgRNAs targeting exon 12 of fIL6ST. In some embodiments, the CRISPR gene-editing system for the treatment of gout comprises one or more sgRNAs targeting exon 13 of fIL6ST. In some embodiments, the CRISPR gene-editing system for the treatment of gout comprises one or more sgRNAs targeting exon 14 of fIL6ST. In some embodiments, the CRISPR gene-editing system for the treatment of gout comprises one or more sgRNAs targeting exon 15 of fIL6ST. In some embodiments, the CRISPR gene-editing system for the treatment of gout comprises one or more sgRNAs targeting exon 16 of fIL6ST. In some embodiments, the CRISPR gene-editing system for the treatment of gout comprises one or more sgRNAs targeting exon 17 of fIL6ST. In some embodiments, the CRISPR gene-editing system for the treatment of gout comprises one or more sgRNAs targeting exon 18 of fIL6ST. In some embodiments, the CRISPR gene-editing system for the treatment of gout comprises one or more sgRNAs targeting exon 19 of fIL6ST. In some embodiments, the CRISPR gene-editing system for the treatment of gout comprises one or more sgRNAs targeting exon 20 of fIL6ST. In some embodiments, the CRISPR gene-editing system for the treatment of gout comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TNFRSF1A. In some embodiments, the CRISPR gene-editing system for the treatment of gout comprises one or more sgRNAs targeting hTNFRSF1A. In some embodiments, the CRISPR gene-editing system for the treatment of gout comprises one or more sgRNAs targeting cTNFRSF1A. In some embodiments, the CRISPR gene-editing system for the treatment of gout comprises one or more sgRNAs targeting eTNFRSF1A. In some embodiments, the CRISPR gene-editing system for the treatment of gout comprises one or more sgRNAs targeting fTNFRSF1A. In some embodiments, the CRISPR gene-editing system for the treatment of gout comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTNFRSF1A. In some embodiments, the CRISPR gene-editing system for the treatment of gout comprises one or more sgRNAs targeting exon 2 of hTNFRSF1A. In some embodiments, the CRISPR gene-editing system for the treatment of gout comprises one or more sgRNAs targeting exon 3 of hTNFRSF1A. In some embodiments, the CRISPR gene-editing system for the treatment of gout comprises one or more sgRNAs targeting exon 4 of hTNFRSF1A. In some embodiments, the CRISPR gene-editing system for the treatment of gout comprises one or more sgRNAs targeting exon 5 of hTNFRSF1A. In some embodiments, the CRISPR gene-editing system for the treatment of gout comprises one or more sgRNAs targeting exon 6 of hTNFRSF1A. In some embodiments, the CRISPR gene-editing system for the treatment of gout comprises one or more sgRNAs targeting exon 7 of hTNFRSF1A. In some embodiments, the CRISPR gene-editing system for the treatment of gout comprises one or more sgRNAs targeting exon 8 of hTNFRSF1A. In some embodiments, the CRISPR gene-editing system for the treatment of gout comprises one or more sgRNAs targeting exon 9 of hTNFRSF1A. In some embodiments, the CRISPR gene-editing system for the treatment of gout comprises one or more sgRNAs targeting exon 10 of hTNFRSF1A. In some embodiments, the CRISPR gene-editing system for the treatment of gout comprises one or more sgRNAs targeting exon 11 of hTNFRSF1A. In some embodiments, the CRISPR gene-editing system for the treatment of gout comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTNFRSF1A. In some embodiments, the CRISPR gene-editing system for the treatment of gout comprises one or more sgRNAs targeting exon 2 of cTNFRSF1A. In some embodiments, the CRISPR gene-editing system for the treatment of gout comprises one or more sgRNAs targeting exon 3 of cTNFRSF1A. In some embodiments, the CRISPR gene-editing system for the treatment of gout comprises one or more sgRNAs targeting exon 4 of cTNFRSF1A. In some embodiments, the CRISPR gene-editing system for the treatment of gout comprises one or more sgRNAs targeting exon 5 of cTNFRSF1A. In some embodiments, the CRISPR gene-editing system for the treatment of gout comprises one or more sgRNAs targeting exon 6 of cTNFRSF1A. In some embodiments, the CRISPR gene-editing system for the treatment of gout comprises one or more sgRNAs targeting exon 7 of cTNFRSF1A. In some embodiments, the CRISPR gene-editing system for the treatment of gout comprises one or more sgRNAs targeting exon 8 of cTNFRSF1A. In some embodiments, the CRISPR gene-editing system for the treatment of gout comprises one or more sgRNAs targeting exon 9 of cTNFRSF1A. In some embodiments, the CRISPR gene-editing system for the treatment of gout comprises one or more sgRNAs targeting exon 10 of cTNFRSF1A. In some embodiments, the CRISPR gene-editing system for the treatment of gout comprises one or more sgRNAs targeting exon 11 of cTNFRSF1A. In some embodiments, the CRISPR gene-editing system for the treatment of gout comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTNFRSF1A. In some embodiments, the CRISPR gene-editing system for the treatment of gout comprises one or more sgRNAs targeting exon 2 of eTNFRSF1A. In some embodiments, the CRISPR gene-editing system for the treatment of gout comprises one or more sgRNAs targeting exon 3 of eTNFRSF1A. In some embodiments, the CRISPR gene-editing system for the treatment of gout comprises one or more sgRNAs targeting exon 4 of eTNFRSF1A. In some embodiments, the CRISPR gene-editing system for the treatment of gout comprises one or more sgRNAs targeting exon 5 of eTNFRSF1A. In some embodiments, the CRISPR gene-editing system for the treatment of gout comprises one or more sgRNAs targeting exon 6 of eTNFRSF1A. In some embodiments, the CRISPR gene-editing system for the treatment of gout comprises one or more sgRNAs targeting exon 7 of eTNFRSF1A. In some embodiments, the CRISPR gene-editing system for the treatment of gout comprises one or more sgRNAs targeting exon 8 of eTNFRSF1A. In some embodiments, the CRISPR gene-editing system for the treatment of gout comprises one or more sgRNAs targeting exon 9 of eTNFRSF1A. In some embodiments, the CRISPR gene-editing system for the treatment of gout comprises one or more sgRNAs targeting exon 10 of eTNFRSF1A. In some embodiments, the CRISPR gene-editing system for the treatment of gout comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTNFRSF1A. In some embodiments, the CRISPR gene-editing system for the treatment of gout comprises one or more sgRNAs targeting exon 2 of fTNFRSF1A. In some embodiments, the CRISPR gene-editing system for the treatment of gout comprises one or more sgRNAs targeting exon 3 of fTNFRSF1A. In some embodiments, the CRISPR gene-editing system for the treatment of gout comprises one or more sgRNAs targeting exon 4 of fTNFRSF1A. In some embodiments, the CRISPR gene-editing system for the treatment of gout comprises one or more sgRNAs targeting exon 5 of fTNFRSF1A. In some embodiments, the CRISPR gene-editing system for the treatment of gout comprises one or more sgRNAs targeting exon 6 of fTNFRSF1A. In some embodiments, the CRISPR gene-editing system for the treatment of gout comprises one or more sgRNAs targeting exon 7 of fTNFRSF1A. In some embodiments, the CRISPR gene-editing system for the treatment of gout comprises one or more sgRNAs targeting exon 8 of fTNFRSF1A. In some embodiments, the CRISPR gene-editing system for the treatment of gout comprises one or more sgRNAs targeting exon 9 of fTNFRSF1A. In some embodiments, the CRISPR gene-editing system for the treatment of gout comprises one or more sgRNAs targeting exon 10 of fTNFRSF1A. In some embodiments, the CRISPR gene-editing system for the treatment of gout comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TNFRSF1B. In some embodiments, the CRISPR gene-editing system for the treatment of gout comprises one or more sgRNAs targeting hTNFRSF1B. In some embodiments, the CRISPR gene-editing system for the treatment of gout comprises one or more sgRNAs targeting cTNFRSF1B. In some embodiments, the CRISPR gene-editing system for the treatment of gout comprises one or more sgRNAs targeting eTNFRSF1B. In some embodiments, the CRISPR gene-editing system for the treatment of gout comprises one or more sgRNAs targeting fTNFRSF1B. In some embodiments, the CRISPR gene-editing system for the treatment of gout comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTNFRSF1B. In some embodiments, the CRISPR gene-editing system for the treatment of gout comprises one or more sgRNAs targeting exon 2 of hTNFRSF1B. In some embodiments, the CRISPR gene-editing system for the treatment of gout comprises one or more sgRNAs targeting exon 3 of hTNFRSF1B. In some embodiments, the CRISPR gene-editing system for the treatment of gout comprises one or more sgRNAs targeting exon 4 of hTNFRSF1B. In some embodiments, the CRISPR gene-editing system for the treatment of gout comprises one or more sgRNAs targeting exon 5 of hTNFRSF1B. In some embodiments, the CRISPR gene-editing system for the treatment of gout comprises one or more sgRNAs targeting exon 6 of hTNFRSF1B. In some embodiments, the CRISPR gene-editing system for the treatment of gout comprises one or more sgRNAs targeting exon 7 of hTNFRSF1B. In some embodiments, the CRISPR gene-editing system for the treatment of gout comprises one or more sgRNAs targeting exon 8 of hTNFRSF1B. In some embodiments, the CRISPR gene-editing system for the treatment of gout comprises one or more sgRNAs targeting exon 9 of hTNFRSF1B. In some embodiments, the CRISPR gene-editing system for the treatment of gout comprises one or more sgRNAs targeting exon 10 of hTNFRSF1B. In some embodiments, the CRISPR gene-editing system for the treatment of gout comprises one or more sgRNAs targeting exon 11 of hTNFRSF1B In some embodiments, the CRISPR gene-editing system for the treatment of gout comprises one or more sgRNAs targeting exon 12 of hTNFRSF1B. In some embodiments, the CRISPR gene-editing system for the treatment of gout comprises one or more sgRNAs targeting exon 13 of hTNFRSF1B. In some embodiments, the CRISPR gene-editing system for the treatment of gout comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTNFRSF1B. In some embodiments, the CRISPR gene-editing system for the treatment of gout comprises one or more sgRNAs targeting exon 2 of cTNFRSF1B. In some embodiments, the CRISPR gene-editing system for the treatment of gout comprises one or more sgRNAs targeting exon 3 of cTNFRSF1B. In some embodiments, the CRISPR gene-editing system for the treatment of gout comprises one or more sgRNAs targeting exon 4 of cTNFRSF1B. In some embodiments, the CRISPR gene-editing system for the treatment of gout comprises one or more sgRNAs targeting exon 5 of cTNFRSF1B. In some embodiments, the CRISPR gene-editing system for the treatment of gout comprises one or more sgRNAs targeting exon 6 of cTNFRSF1B. In some embodiments, the CRISPR gene-editing system for the treatment of gout comprises one or more sgRNAs targeting exon 7 of cTNFRSF1B. In some embodiments, the CRISPR gene-editing system for the treatment of gout comprises one or more sgRNAs targeting exon 8 of cTNFRSF1B. In some embodiments, the CRISPR gene-editing system for the treatment of gout comprises one or more sgRNAs targeting exon 9 of cTNFRSF1B. In some embodiments, the CRISPR gene-editing system for the treatment of gout comprises one or more sgRNAs targeting exon 10 of cTNFRSF1B. In some embodiments, the CRISPR gene-editing system for the treatment of gout comprises one or more sgRNAs targeting exon 11 of cTNFRSF1B. In some embodiments, the CRISPR gene-editing system for the treatment of gout comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTNFRSF1B. In some embodiments, the CRISPR gene-editing system for the treatment of gout comprises one or more sgRNAs targeting exon 2 of eTNFRSF1B. In some embodiments, the CRISPR gene-editing system for the treatment of gout comprises one or more sgRNAs targeting exon 3 of eTNFRSF1B. In some embodiments, the CRISPR gene-editing system for the treatment of gout comprises one or more sgRNAs targeting exon 4 of eTNFRSF1B. In some embodiments, the CRISPR gene-editing system for the treatment of gout comprises one or more sgRNAs targeting exon 5 of eTNFRSF1B. In some embodiments, the CRISPR gene-editing system for the treatment of gout comprises one or more sgRNAs targeting exon 6 of eTNFRSF1B. In some embodiments, the CRISPR gene-editing system for the treatment of gout comprises one or more sgRNAs targeting exon 7 of eTNFRSF1B. In some embodiments, the CRISPR gene-editing system for the treatment of gout comprises one or more sgRNAs targeting exon 8 of eTNFRSF1B. In some embodiments, the CRISPR gene-editing system for the treatment of gout comprises one or more sgRNAs targeting exon 9 of eTNFRSF1B. In some embodiments, the CRISPR gene-editing system for the treatment of gout comprises one or more sgRNAs targeting exon 10 of eTNFRSF1B. In some embodiments, the CRISPR gene-editing system for the treatment of gout comprises one or more sgRNAs targeting exon 11 of eTNFRSF1B. In some embodiments, the CRISPR gene-editing system for the treatment of gout comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTNFRSF1B. In some embodiments, the CRISPR gene-editing system for the treatment of gout comprises one or more sgRNAs targeting exon 2 of fTNFRSF1B. In some embodiments, the CRISPR gene-editing system for the treatment of gout comprises one or more sgRNAs targeting exon 3 of fTNFRSF1B. In some embodiments, the CRISPR gene-editing system for the treatment of gout comprises one or more sgRNAs targeting exon 4 of fTNFRSF1B. In some embodiments, the CRISPR gene-editing system for the treatment of gout comprises one or more sgRNAs targeting exon 5 of fTNFRSF1B. In some embodiments, the CRISPR gene-editing system for the treatment of gout comprises one or more sgRNAs targeting exon 6 of fTNFRSF1B. In some embodiments, the CRISPR gene-editing system for the treatment of gout comprises one or more sgRNAs targeting exon 7 of fTNFRSF1B. In some embodiments, the CRISPR gene-editing system for the treatment of gout comprises one or more sgRNAs targeting exon 8 of fTNFRSF1B. In some embodiments, the CRISPR gene-editing system for the treatment of gout comprises one or more sgRNAs targeting exon 9 of fTNFRSF1B. In some embodiments, the CRISPR gene-editing system for the treatment of gout comprises one or more sgRNAs targeting exon 10 of fTNFRSF1B. In some embodiments, the CRISPR gene-editing system for the treatment of gout comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TNFRSF3. In some embodiments, the CRISPR gene-editing system for the treatment of gout comprises one or more sgRNAs targeting hTNFRSF3. In some embodiments, the CRISPR gene-editing system for the treatment of gout comprises one or more sgRNAs targeting cTNFRSF3. In some embodiments, the CRISPR gene-editing system for the treatment of gout comprises one or more sgRNAs targeting eTNFRSF3. In some embodiments, the CRISPR gene-editing system for the treatment of gout comprises one or more sgRNAs targeting fTNFRSF3. In some embodiments, the CRISPR gene-editing system for the treatment of gout comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTNFRSF3. In some embodiments, the CRISPR gene-editing system for the treatment of gout comprises one or more sgRNAs targeting exon 2 of hTNFRSF3. In some embodiments, the CRISPR gene-editing system for the treatment of gout comprises one or more sgRNAs targeting exon 3 of hTNFRSF3. In some embodiments, the CRISPR gene-editing system for the treatment of gout comprises one or more sgRNAs targeting exon 4 of hTNFRSF3. In some embodiments, the CRISPR gene-editing system for the treatment of gout comprises one or more sgRNAs targeting exon 5 of hTNFRSF3. In some embodiments, the CRISPR gene-editing system for the treatment of gout comprises one or more sgRNAs targeting exon 6 of hTNFRSF3. In some embodiments, the CRISPR gene-editing system for the treatment of gout comprises one or more sgRNAs targeting exon 7 of hTNFRSF3. In some embodiments, the CRISPR gene-editing system for the treatment of gout comprises one or more sgRNAs targeting exon 8 of hTNFRSF3. In some embodiments, the CRISPR gene-editing system for the treatment of gout comprises one or more sgRNAs targeting exon 9 of hTNFRSF3. In some embodiments, the CRISPR gene-editing system for the treatment of gout comprises one or more sgRNAs targeting exon 10 of hTNFRSF3. In some embodiments, the CRISPR gene-editing system for the treatment of gout comprises one or more sgRNAs targeting exon 11 of hTNFRSF3. In some embodiments, the CRISPR gene-editing system for the treatment of gout comprises one or more sgRNAs targeting exon 12 of hTNFRSF3. In some embodiments, the CRISPR gene-editing system for the treatment of gout comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTNFRSF3. In some embodiments, the CRISPR gene-editing system for the treatment of gout comprises one or more sgRNAs targeting exon 2 of cTNFRSF3. In some embodiments, the CRISPR gene-editing system for the treatment of gout comprises one or more sgRNAs targeting exon 3 of cTNFRSF3. In some embodiments, the CRISPR gene-editing system for the treatment of gout comprises one or more sgRNAs targeting exon 4 of cTNFRSF3. In some embodiments, the CRISPR gene-editing system for the treatment of gout comprises one or more sgRNAs targeting exon 5 of cTNFRSF3. In some embodiments, the CRISPR gene-editing system for the treatment of gout comprises one or more sgRNAs targeting exon 6 of cTNFRSF3. In some embodiments, the CRISPR gene-editing system for the treatment of gout comprises one or more sgRNAs targeting exon 7 of cTNFRSF3. In some embodiments, the CRISPR gene-editing system for the treatment of gout comprises one or more sgRNAs targeting exon 8 of cTNFRSF3. In some embodiments, the CRISPR gene-editing system for the treatment of gout comprises one or more sgRNAs targeting exon 9 of cTNFRSF3. In some embodiments, the CRISPR gene-editing system for the treatment of gout comprises one or more sgRNAs targeting exon 10 of cTNFRSF3. In some embodiments, the CRISPR gene-editing system for the treatment of gout comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTNFRSF3. In some embodiments, the CRISPR gene-editing system for the treatment of gout comprises one or more sgRNAs targeting exon 2 of eTNFRSF3. In some embodiments, the CRISPR gene-editing system for the treatment of gout comprises one or more sgRNAs targeting exon 3 of eTNFRSF3. In some embodiments, the CRISPR gene-editing system for the treatment of gout comprises one or more sgRNAs targeting exon 4 of eTNFRSF3. In some embodiments, the CRISPR gene-editing system for the treatment of gout comprises one or more sgRNAs targeting exon 5 of eTNFRSF3. In some embodiments, the CRISPR gene-editing system for the treatment of gout comprises one or more sgRNAs targeting exon 6 of eTNFRSF3. In some embodiments, the CRISPR gene-editing system for the treatment of gout comprises one or more sgRNAs targeting exon 7 of eTNFRSF3. In some embodiments, the CRISPR gene-editing system for the treatment of gout comprises one or more sgRNAs targeting exon 8 of eTNFRSF3. In some embodiments, the CRISPR gene-editing system for the treatment of gout comprises one or more sgRNAs targeting exon 9 of eTNFRSF3. In some embodiments, the CRISPR gene-editing system for the treatment of gout comprises one or more sgRNAs targeting exon 10 of eTNFRSF3. In some embodiments, the CRISPR gene-editing system for the treatment of gout comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTNFRSF3. In some embodiments, the CRISPR gene-editing system for the treatment of gout comprises one or more sgRNAs targeting exon 2 of fTNFRSF3. In some embodiments, the CRISPR gene-editing system for the treatment of gout comprises one or more sgRNAs targeting exon 3 of fTNFRSF3. In some embodiments, the CRISPR gene-editing system for the treatment of gout comprises one or more sgRNAs targeting exon 4 of fTNFRSF3. In some embodiments, the CRISPR gene-editing system for the treatment of gout comprises one or more sgRNAs targeting exon 5 of fTNFRSF3. In some embodiments, the CRISPR gene-editing system for the treatment of gout comprises one or more sgRNAs targeting exon 6 of fTNFRSF3. In some embodiments, the CRISPR gene-editing system for the treatment of gout comprises one or more sgRNAs targeting exon 7 of fTNFRSF3. In some embodiments, the CRISPR gene-editing system for the treatment of gout comprises one or more sgRNAs targeting exon 8 of fTNFRSF3. In some embodiments, the CRISPR gene-editing system for the treatment of gout comprises one or more sgRNAs targeting exon 9 of fTNFRSF3. In some embodiments, the CRISPR gene-editing system for the treatment of gout comprises one or more sgRNAs targeting exon 10 of fTNFRSF3. In some embodiments, the CRISPR gene-editing system for the treatment of gout comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TNFRSF4. In some embodiments, the CRISPR gene-editing system for the treatment of gout comprises one or more sgRNAs targeting hTNFRSF4. In some embodiments, the CRISPR gene-editing system for the treatment of gout comprises one or more sgRNAs targeting cTNFRSF4. In some embodiments, the CRISPR gene-editing system for the treatment of gout comprises one or more sgRNAs targeting eTNFRSF4. In some embodiments, the CRISPR gene-editing system for the treatment of gout comprises one or more sgRNAs targeting fTNFRSF4. In some embodiments, the CRISPR gene-editing system for the treatment of gout comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTNFRSF4. In some embodiments, the CRISPR gene-editing system for the treatment of gout comprises one or more sgRNAs targeting exon 2 of hTNFRSF4. In some embodiments, the CRISPR gene-editing system for the treatment of gout comprises one or more sgRNAs targeting exon 3 of hTNFRSF4. In some embodiments, the CRISPR gene-editing system for the treatment of gout comprises one or more sgRNAs targeting exon 4 of hTNFRSF4. In some embodiments, the CRISPR gene-editing system for the treatment of gout comprises one or more sgRNAs targeting exon 5 of hTNFRSF4. In some embodiments, the CRISPR gene-editing system for the treatment of gout comprises one or more sgRNAs targeting exon 6 of hTNFRSF4. In some embodiments, the CRISPR gene-editing system for the treatment of gout comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTNFRSF4. In some embodiments, the CRISPR gene-editing system for the treatment of gout comprises one or more sgRNAs targeting exon 2 of cTNFRSF4. In some embodiments, the CRISPR gene-editing system for the treatment of gout comprises one or more sgRNAs targeting exon 3 of cTNFRSF4. In some embodiments, the CRISPR gene-editing system for the treatment of gout comprises one or more sgRNAs targeting exon 4 of cTNFRSF4. In some embodiments, the CRISPR gene-editing system for the treatment of gout comprises one or more sgRNAs targeting exon 5 of cTNFRSF4. In some embodiments, the CRISPR gene-editing system for the treatment of gout comprises one or more sgRNAs targeting exon 6 of cTNFRSF4. In some embodiments, the CRISPR gene-editing system for the treatment of gout comprises one or more sgRNAs targeting exon 7 of cTNFRSF4. In some embodiments, the CRISPR gene-editing system for the treatment of gout comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTNFRSF4. In some embodiments, the CRISPR gene-editing system for the treatment of gout comprises one or more sgRNAs targeting exon 2 of eTNFRSF4. In some embodiments, the CRISPR gene-editing system for the treatment of gout comprises one or more sgRNAs targeting exon 3 of eTNFRSF4. In some embodiments, the CRISPR gene-editing system for the treatment of gout comprises one or more sgRNAs targeting exon 4 of eTNFRSF4. In some embodiments, the CRISPR gene-editing system for the treatment of gout comprises one or more sgRNAs targeting exon 5 of eTNFRSF4. In some embodiments, the CRISPR gene-editing system for the treatment of gout comprises one or more sgRNAs targeting exon 6 of eTNFRSF4. In some embodiments, the CRISPR gene-editing system for the treatment of gout comprises one or more sgRNAs targeting exon 7 of eTNFRSF4. In some embodiments, the CRISPR gene-editing system for the treatment of gout comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of fTNFRSF4. In some embodiments, the CRISPR gene-editing system for the treatment of gout comprises one or more sgRNAs targeting exon 2 of fTNFRSF4. In some embodiments, the CRISPR gene-editing system for the treatment of gout comprises one or more sgRNAs targeting exon 3 of fTNFRSF4. In some embodiments, the CRISPR gene-editing system for the treatment of gout comprises one or more sgRNAs targeting exon 4 of fTNFRSF4. In some embodiments, the CRISPR gene-editing system for the treatment of gout comprises one or more sgRNAs targeting exon 5 of fTNFRSF4. In some embodiments, the CRISPR gene-editing system for the treatment of gout comprises one or more sgRNAs targeting exon 6 of fTNFRSF4. In some embodiments, the CRISPR gene-editing system for the treatment of gout comprises one or more sgRNAs targeting exon 7 of fTNFRSF4. In some embodiments, the CRISPR gene-editing system for the treatment of gout comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting TNFRSF11A. In some embodiments, the CRISPR gene-editing system for the treatment of gout comprises one or more sgRNAs targeting hTNFRSF11A. In some embodiments, the CRISPR gene-editing system for the treatment of gout comprises one or more sgRNAs targeting cTNFRSF11A. In some embodiments, the CRISPR gene-editing system for the treatment of gout comprises one or more sgRNAs targeting eTNFRSF11A. In some embodiments, the CRISPR gene-editing system for the treatment of gout comprises one or more sgRNAs targeting fTNFRSF11A. In some embodiments, the CRISPR gene-editing system for the treatment of gout comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of hTNFRSF11A. In some embodiments, the CRISPR gene-editing system for the treatment of gout comprises one or more sgRNAs targeting exon 2 of hTNFRSF11A. In some embodiments, the CRISPR gene-editing system for the treatment of gout comprises one or more sgRNAs targeting exon 3 of hTNFRSF11A. In some embodiments, the CRISPR gene-editing system for the treatment of gout comprises one or more sgRNAs targeting exon 4 of hTNFRSF11A. In some embodiments, the CRISPR gene-editing system for the treatment of gout comprises one or more sgRNAs targeting exon 5 of hTNFRSF11A. In some embodiments, the CRISPR gene-editing system for the treatment of gout comprises one or more sgRNAs targeting exon 6 of hTNFRSF11A. In some embodiments, the CRISPR gene-editing system for the treatment of gout comprises one or more sgRNAs targeting exon 7 of hTNFRSF11A. In some embodiments, the CRISPR gene-editing system for the treatment of gout comprises one or more sgRNAs targeting exon 8 of hTNFRSF11A. In some embodiments, the CRISPR gene-editing system for the treatment of gout comprises one or more sgRNAs targeting exon 9 of hTNFRSF11A. In some embodiments, the CRISPR gene-editing system for the treatment of gout comprises one or more sgRNAs targeting exon 10 of hTNFRSF11A. In some embodiments, the CRISPR gene-editing system for the treatment of gout comprises one or more sgRNAs targeting exon 11 of hTNFRSF11A. In some embodiments, the CRISPR gene-editing system for the treatment of gout comprises one or more sgRNAs targeting exon 12 of hTNFRSF11A. In some embodiments, the CRISPR gene-editing system for the treatment of gout comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of cTNFRSF11A. In some embodiments, the CRISPR gene-editing system for the treatment of gout comprises one or more sgRNAs targeting exon 2 of cTNFRSF11A. In some embodiments, the CRISPR gene-editing system for the treatment of gout comprises one or more sgRNAs targeting exon 3 of cTNFRSF11A. In some embodiments, the CRISPR gene-editing system for the treatment of gout comprises one or more sgRNAs targeting exon 4 of cTNFRSF11A. In some embodiments, the CRISPR gene-editing system for the treatment of gout comprises one or more sgRNAs targeting exon 5 of cTNFRSF11A. In some embodiments, the CRISPR gene-editing system for the treatment of gout comprises one or more sgRNAs targeting exon 6 of cTNFRSF11A. In some embodiments, the CRISPR gene-editing system for the treatment of gout comprises one or more sgRNAs targeting exon 7 of cTNFRSF11A. In some embodiments, the CRISPR gene-editing system for the treatment of gout comprises one or more sgRNAs targeting exon 8 of cTNFRSF11A. In some embodiments, the CRISPR gene-editing system for the treatment of gout comprises one or more sgRNAs targeting exon 9 of cTNFRSF11A. In some embodiments, the CRISPR gene-editing system for the treatment of gout comprises one or more sgRNAs targeting exon 10 of cTNFRSF11A. In some embodiments, the CRISPR gene-editing system for the treatment of gout comprises one or more sgRNAs targeting exon 11 of cTNFRSF11A. In some embodiments, the CRISPR gene-editing system for the treatment of gout comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon 1 of eTNFRSF11A. In some embodiments, the CRISPR gene-editing system for the treatment of gout comprises one or more sgRNAs targeting exon 2 of eTNFRSF11A. In some embodiments, the CRISPR gene-editing system for the treatment of gout comprises one or more sgRNAs targeting exon 3 of eTNFRSF11A. In some embodiments, the CRISPR gene-editing system for the treatment of gout comprises one or more sgRNAs targeting exon 4 of eTNFRSF11A. In some embodiments, the CRISPR gene-editing system for the treatment of gout comprises one or more sgRNAs targeting exon 5 of eTNFRSF11A. In some embodiments, the CRISPR gene-editing system for the treatment of gout comprises one or more sgRNAs targeting exon 6 of eTNFRSF11A. In some embodiments, the CRISPR gene-editing system for the treatment of gout comprises one or more sgRNAs targeting exon 7 of eTNFRSF11A. In some embodiments, the CRISPR gene-editing system for the treatment of gout comprises one or more sgRNAs targeting exon 8 of eTNFRSF11A. In some embodiments, the CRISPR gene-editing system for the treatment of gout comprises one or more sgRNAs targeting exon 9 of eTNFRSF11A. In some embodiments, the CRISPR gene-editing system for the treatment of gout comprises one or more sgRNAs targeting exon 10 of eTNFRSF11A. In some embodiments, the CRISPR gene-editing system for the treatment of gout comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of gout comprises one or more sgRNAs targeting exon t of fTNFRSF11A. In some embodiments, the CRISPR gene-editing system for the treatment of gout comprises one or more sgRNAs targeting exon 2 of fTNFRSF11A. In some embodiments, the CRISPR gene-editing system for the treatment of gout comprises one or more sgRNAs targeting exon 3 of fTNFRSF11A. In some embodiments, the CRISPR gene-editing system for the treatment of gout comprises one or more sgRNAs targeting exon 4 of fTNFRSF11A. In some embodiments, the CRISPR gene-editing system for the treatment of gout comprises one or more sgRNAs targeting exon 5 of fTNFRSF11A. In some embodiments, the CRISPR gene-editing system for the treatment of gout comprises one or more sgRNAs targeting exon 6 of fTNFRSF11A. In some embodiments, the CRISPR gene-editing system for the treatment of gout comprises one or more sgRNAs targeting exon 7 of fTNFRSF11A. In some embodiments, the CRISPR gene-editing system for the treatment of gout comprises one or more sgRNAs targeting exon 8 of fTNFRSF11A. In some embodiments, the CRISPR gene-editing system for the treatment of gout comprises one or more sgRNAs targeting exon 9 of fTNFRSF11A. In some embodiments, the CRISPR gene-editing system for the treatment of gout comprises one or more sgRNAs targeting exon 10 of fTNFRSF11A. In some embodiments, the CRISPR gene-editing system for the treatment of gout comprises one or more sgRNAs targeting exon 11 of fTNFRSF11A. In some embodiments, the CRISPR gene-editing system for the treatment of gout comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### V. Back or Spine Conditions or Disorders

### A. Introduction

Back or spine conditions or disorders, including low back pain, cervical pain, sacral pain, thoracic pain. and pain or inflammation associated with discogenic disorders e.g., degenerative disc disease (DDD) or internal disc disruption (IDD), are a major cause of morbidity and disability worldwide for which few long-term options for amelioration currently exist. Andersson GB. Epidemiological features of chronic low-back pain. Lancet. 1999; 354:581-585. Presently available treatments include surgical or less invasive options that often fail to offer long-term palliation. Ju, et al. Global Spine Journal (2020): 2192568220963058. All vertebrate species are affected by back or spine conditions or disorders, including working animals, domestic pets, and their owners. All suffer from the associated discomfort, pain, and disability, depending on the degree of disease progression.

Back or spine conditions or disorders, such as low back pain, are complex diseases characterized by a multitude of inputs contributing to a progressive course of disability. Among these contributors are morphological irregularities (e.g., disc disruptions), inflammation, changes in the localized cellular environment (e.g., vascularization and/or innervation) and degenerative changes. Peng, Bao-Gan. World Journal of Orthopedics 4.2 (2013): 42. Each contributing factor is driven by differential expression of various gene products, including at least pro-inflammatory cytokines, growth factors, pain signaling molecules, and other effector biomolecules. There is a pressing need for new methods and compositions to treat this spectrum of disease and its associated disability.

The present disclosure provides compositions and methods for back or spine conditions or disorders. Particularly, said conditions are treated by reducing pro-inflammatory signaling mediated by inflammatory cytokines, such as, IL1α, IL1β, TNF-α, IL6, IL8, IL18, IL33, matrix metalloproteinases (MMPs), or TGFB1, or TGFB2, binding to their cognate receptor(s). In some embodiments, such conditions or disorders include disorders of the intervertebral discs (IVDs). In some embodiments, the condition or disorder is DDD. In some embodiments, the condition or disorder is IDD. In some embodiments, the condition or disorder is low back pain. Some embodiments are further useful as it relates to encapsulation in an LNP system described herein, e.g., and without limitation, any one of LNP systems LNP001 to LNP240.

Among the advantages of the present disclosure over treatments currently available for mammals afflicted with back or spine conditions or disorders include the period of relief from symptoms. Upon local administration (e.g., intradiscal injection) and subsequent genetic editing of a cell (e.g., a chondrocyte, a tenocyte, an osteocyte, a monocyte, a macrophage or the cells of the nucleus pulposus or annulus fibrosus), pro-inflammatory signaling is silenced through the targeted gene for the life of that cell. By contrast, biologic treatments require periodic dosing, which may magnify the impact of any side effects, which can be severe. Among various genetic approaches, the present disclosure is also superior due to the resistance to leakiness built in by virtue of modifying a protein receptor, rather than ablating its expression altogether.

### B. Low Back Pain

In one aspect, the present disclosure encompasses treatments for low back pain. In some embodiments, the low back pain treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the low back pain treatment comprises a CRISPR gene-editing system targeting hIL1R1. In some embodiments, the low back pain treatment comprises a CRISPR gene-editing system targeting cIL1R1. In some embodiments, the low back pain treatment comprises a CRISP gene-editing system R targeting eIL1R1. In some embodiments, the low back pain treatment comprises a CRISPR gene-editing system targeting fIL1R1. In some embodiments, the CRISPR gene-editing system for the treatment of low back pain comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hIL1R1. In some embodiments, the CRISPR gene-editing system for the treatment of low back pain comprises one or more sgRNAs targeting exon 2 of hIL1R1. In some embodiments, the CRISPR gene-editing system for the treatment of low back pain comprises one or more sgRNAs targeting exon 3 of hIL1R1. In some embodiments, the CRISPR gene-editing system for the treatment of low back pain comprises one or more sgRNAs targeting exon 4 of hIL1R1. In some embodiments, the CRISPR gene-editing system for the treatment of low back pain comprises one or more sgRNAs targeting exon 5 of hIL1R1. In some embodiments, the CRISPR gene-editing system for the treatment of low back pain comprises one or more sgRNAs targeting exon 6 of hIL1R1. In some embodiments, the CRISPR gene-editing system for the treatment of low back pain comprises one or more sgRNAs targeting exon 7 of hIL1R1. In some embodiments, the CRISPR gene-editing system for the treatment of low back pain comprises one or more sgRNAs targeting exon 8 of hIL1R1. In some embodiments, the CRISPR gene-editing system for the treatment of low back pain comprises one or more sgRNAs targeting exon 9 of hIL1R1. In some embodiments, the CRISPR gene-editing system for the treatment of low back pain comprises one or more sgRNAs targeting exon 10 of hIL1R1. In some embodiments, the CRISPR gene-editing system for the treatment of low back pain comprises one or more sgRNAs targeting exon 11 of hIL1R1. In some embodiments, the CRISPR gene-editing system for the treatment of low back pain comprises one or more sgRNAs targeting exon 12 of hIL1R1. In some embodiments, the CRISPR gene-editing system for the treatment of low back pain comprises one or more sgRNAs targeting exon 13 of hIL1R1. In some embodiments, the CRISPR gene-editing system for the treatment of low back pain comprises one or more sgRNAs targeting exon 14 of hIL1R1. In some embodiments, the CRISPR gene-editing system for the treatment of low back pain comprises one or more sgRNAs targeting exon 15 of hIL1R1. In some embodiments, the CRISPR gene-editing system for the treatment of low back pain comprises one or more sgRNAs targeting exon 16 of hIL1R1. In some embodiments, the CRISPR gene-editing system for the treatment of low back pain comprises one or more sgRNAs targeting exon 17 of hIL1R1. In some embodiments, the CRISPR gene-editing system for the treatment of low back pain comprises one or more sgRNAs targeting exon 18 of hIL1R1. In some embodiments, the CRISPR gene-editing system for the treatment of low back pain comprises one or more sgRNAs targeting exon 19 of hIL1R1. In some embodiments, the CRISPR gene-editing system for the treatment of low back pain comprises one or more sgRNAs targeting exon 20 of hIL1R1. In some embodiments, the CRISPR gene-editing system for the treatment of low back pain comprises one or more sgRNAs targeting exon 21 of hIL1R1. In some embodiments, the CRISPR gene-editing system for the treatment of low back pain comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cIL1R1. In some embodiments, the CRISPR gene-editing system for the treatment of low back pain comprises one or more sgRNAs targeting exon 2 of cIL1R1. In some embodiments, the CRISPR gene-editing system for the treatment of low back pain comprises one or more sgRNAs targeting exon 3 of cIL1R1. In some embodiments, the CRISPR gene-editing system for the treatment of low back pain comprises one or more sgRNAs targeting exon 4 of cIL1R1. In some embodiments, the CRISPR gene-editing system for the treatment of low back pain comprises one or more sgRNAs targeting exon 5 of cIL1R1. In some embodiments, the CRISPR gene-editing system for the treatment of low back pain comprises one or more sgRNAs targeting exon 6 of cIL1R1. In some embodiments, the CRISPR gene-editing system for the treatment of low back pain comprises one or more sgRNAs targeting exon 7 of cIL1R1. In some embodiments, the CRISPR gene-editing system for the treatment of low back pain comprises one or more sgRNAs targeting exon 8 of cIL1R1. In some embodiments, the CRISPR gene-editing system for the treatment of low back pain comprises one or more sgRNAs targeting exon 9 of cIL1R1. In some embodiments, the CRISPR gene-editing system for the treatment of low back pain comprises one or more sgRNAs targeting exon 10 of cIL1R1. In some embodiments, the CRISPR gene-editing system for the treatment of low back pain comprises one or more sgRNAs targeting exon 11 of cIL1R1. In some embodiments, the CRISPR gene-editing system for the treatment of low back pain comprises one or more sgRNAs targeting exon 12 of cIL1R1. In some embodiments, the CRISPR gene-editing system for the treatment of low back pain comprises one or more sgRNAs targeting exon 13 of cIL1R1. In some embodiments, the CRISPR gene-editing system for the treatment of low back pain comprises one or more sgRNAs targeting exon 14 of cIL1R1. In some embodiments, the CRISPR gene-editing system for the treatment of low back pain comprises one or more sgRNAs targeting exon 15 of cIL1R1. In some embodiments, the CRISPR gene-editing system for the treatment of low back pain comprises one or more sgRNAs targeting exon 16 of cIL1R1. In some embodiments, the CRISPR gene-editing system for the treatment of low back pain comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eIL1R1. In some embodiments, the CRISPR gene-editing system for the treatment of low back pain comprises one or more sgRNAs targeting exon 2 of eIL1R1. In some embodiments, the CRISPR gene-editing system for the treatment of low back pain comprises one or more sgRNAs targeting exon 3 of eIL1R1. In some embodiments, the CRISPR gene-editing system for the treatment of low back pain comprises one or more sgRNAs targeting exon 4 of eIL1R1. In some embodiments, the CRISPR gene-editing system for the treatment of low back pain comprises one or more sgRNAs targeting exon 5 of eIL1R1. In some embodiments, the CRISPR gene-editing system for the treatment of low back pain comprises one or more sgRNAs targeting exon 6 of eIL1R1. In some embodiments, the CRISPR gene-editing system for the treatment of low back pain comprises one or more sgRNAs targeting exon 7 of eIL1R1. In some embodiments, the CRISPR gene-editing system for the treatment of low back pain comprises one or more sgRNAs targeting exon 8 of eIL1R1. In some embodiments, the CRISPR gene-editing system for the treatment of low back pain comprises one or more sgRNAs targeting exon 9 of eIL1R1. In some embodiments, the CRISPR gene-editing system for the treatment of low back pain comprises one or more sgRNAs targeting exon 10 of eIL1R1. In some embodiments, the CRISPR gene-editing system for the treatment of low back pain comprises one or more sgRNAs targeting exon 11 of eIL1R1. In some embodiments, the CRISPR gene-editing system for the treatment of low back pain comprises one or more sgRNAs targeting exon 12 of eIL1R1. In some embodiments, the CRISPR gene-editing system for the treatment of low back pain comprises one or more sgRNAs targeting exon 13 of eIL1R1. In some embodiments, the CRISPR gene-editing system for the treatment of low back pain comprises one or more sgRNAs targeting exon 14 of eIL1R1. In some embodiments, the CRISPR gene-editing system for the treatment of low back pain comprises one or more sgRNAs targeting exon 15 of eIL1R1. In some embodiments, the CRISPR gene-editing system for the treatment of low back pain comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fIL1R1. In some embodiments, the CRISPR gene-editing system for the treatment of low back pain comprises one or more sgRNAs targeting exon 2 of fIL1R1. In some embodiments, the CRISPR gene-editing system for the treatment of low back pain comprises one or more sgRNAs targeting exon 3 of fIL1R1. In some embodiments, the CRISPR gene-editing system for the treatment of low back pain comprises one or more sgRNAs targeting exon 4 of fIL1R1. In some embodiments, the CRISPR gene-editing system for the treatment of low back pain comprises one or more sgRNAs targeting exon 5 of fIL1R1. In some embodiments, the CRISPR gene-editing system for the treatment of low back pain comprises one or more sgRNAs targeting exon 6 of fIL1R1. In some embodiments, the CRISPR gene-editing system for the treatment of low back pain comprises one or more sgRNAs targeting exon 7 of fIL1R1. In some embodiments, the CRISPR gene-editing system for the treatment of low back pain comprises one or more sgRNAs targeting exon 8 of fIL1R1. In some embodiments, the CRISPR gene-editing system for the treatment of low back pain comprises one or more sgRNAs targeting exon 9 of fIL1R1. In some embodiments, the CRISPR gene-editing system for the treatment of low back pain comprises one or more sgRNAs targeting exon 10 of fIL1R1. In some embodiments, the CRISPR gene-editing system for the treatment of low back pain comprises one or more sgRNAs targeting exon 11 of fIL1R1. In some embodiments, the CRISPR gene-editing system for the treatment of low back pain comprises one or more sgRNAs targeting exon 12 of fIL1R1. In some embodiments, the CRISPR gene-editing system for the treatment of low back pain comprises one or more sgRNAs targeting exon 13 of fIL1R1. In some embodiments, the CRISPR gene-editing system for the treatment of low back pain comprises one or more sgRNAs targeting exon 14 of fIL1R1. In some embodiments, the CRISPR gene-editing system for the treatment of low back pain comprises one or more sgRNAs targeting exon 15 of fIL1R1. In some embodiments, the CRISPR gene-editing system for the treatment of low back pain comprises one or more sgRNAs targeting exon 16 of fIL1R1. In some embodiments, the CRISPR gene-editing system for the treatment of low back pain comprises one or more sgRNAs targeting exon 17 of fIL1R1. In some embodiments, the CRISPR gene-editing system for the treatment of low back pain comprises one or more sgRNAs targeting exon 18 of fIL1R1. In some embodiments, the CRISPR gene-editing system for the treatment of low back pain comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting IL1RAP. In some embodiments, the CRISPR gene-editing system for the treatment of low back pain comprises one or more sgRNAs targeting hIL1RAP. In some embodiments, the CRISPR gene-editing system for the treatment of low back pain comprises one or more sgRNAs targeting cIL1RAP. In some embodiments, the CRISPR gene-editing system for the treatment of low back pain comprises one or more sgRNAs targeting eIL1RAP. In some embodiments, the CRISPR gene-editing system for the treatment of low back pain comprises one or more sgRNAs targeting fIL1RAP. In some embodiments, the CRISPR gene-editing system for the treatment of low back pain comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hIL1RAP. In some embodiments, the CRISPR gene-editing system for the treatment of low back pain comprises one or more sgRNAs targeting exon 2 of hIL1RAP. In some embodiments, the CRISPR gene-editing system for the treatment of low back pain comprises one or more sgRNAs targeting exon 3 of hIL1RAP. In some embodiments, the CRISPR gene-editing system for the treatment of low back pain comprises one or more sgRNAs targeting exon 4 of hIL1RAP. In some embodiments, the CRISPR gene-editing system for the treatment of low back pain comprises one or more sgRNAs targeting exon 5 of hIL1RAP. In some embodiments, the CRISPR gene-editing system for the treatment of low back pain comprises one or more sgRNAs targeting exon 6 of hIL1RAP. In some embodiments, the CRISPR gene-editing system for the treatment of low back pain comprises one or more sgRNAs targeting exon 7 of hIL1RAP. In some embodiments, the CRISPR gene-editing system for the treatment of low back pain comprises one or more sgRNAs targeting exon 8 of hIL1RAP. In some embodiments, the CRISPR gene-editing system for the treatment of low back pain comprises one or more sgRNAs targeting exon 9 of hIL1RAP. In some embodiments, the CRISPR gene-editing system for the treatment of low back pain comprises one or more sgRNAs targeting exon 10 of hIL1RAP. In some embodiments, the CRISPR gene-editing system for the treatment of low back pain comprises one or more sgRNAs targeting exon 11 of hIL1RAP. In some embodiments, the CRISPR gene-editing system for the treatment of low back pain comprises one or more sgRNAs targeting exon 12 of hIL1RAP. In some embodiments, the CRISPR gene-editing system for the treatment of low back pain comprises one or more sgRNAs targeting exon 13 of hIL1RAP. In some embodiments, the CRISPR gene-editing system for the treatment of low back pain comprises one or more sgRNAs targeting exon 14 of hIL1RAP. In some embodiments, the CRISPR gene-editing system for the treatment of low back pain comprises one or more sgRNAs targeting exon 15 of hIL1RAP. In some embodiments, the CRISPR gene-editing system for the treatment of low back pain comprises one or more sgRNAs targeting exon 16 of hIL1RAP. In some embodiments, the CRISPR gene-editing system for the treatment of low back pain comprises one or more sgRNAs targeting exon 17 of hIL1RAP. In some embodiments, the CRISPR gene-editing system for the treatment of low back pain comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cIL1RAP. In some embodiments, the CRISPR gene-editing system for the treatment of low back pain comprises one or more sgRNAs targeting exon 2 of cIL1RAP. In some embodiments, the CRISPR gene-editing system for the treatment of low back pain comprises one or more sgRNAs targeting exon 3 of cIL1RAP. In some embodiments, the CRISPR gene-editing system for the treatment of low back pain comprises one or more sgRNAs targeting exon 4 of cIL1RAP. In some embodiments, the CRISPR gene-editing system for the treatment of low back pain comprises one or more sgRNAs targeting exon 5 of cIL1RAP. In some embodiments, the CRISPR gene-editing system for the treatment of low back pain comprises one or more sgRNAs targeting exon 6 of cIL1RAP. In some embodiments, the CRISPR gene-editing system for the treatment of low back pain comprises one or more sgRNAs targeting exon 7 of cIL1RAP. In some embodiments, the CRISPR gene-editing system for the treatment of low back pain comprises one or more sgRNAs targeting exon 8 of cIL1RAP. In some embodiments, the CRISPR gene-editing system for the treatment of low back pain comprises one or more sgRNAs targeting exon 9 of cIL1RAP. In some embodiments, the CRISPR gene-editing system for the treatment of low back pain comprises one or more sgRNAs targeting exon 10 of cIL1RAP. In some embodiments, the CRISPR gene-editing system for the treatment of low back pain comprises one or more sgRNAs targeting exon 11 of cIL1RAP. In some embodiments, the CRISPR gene-editing system for the treatment of low back pain comprises one or more sgRNAs targeting exon 12 of cIL1RAP. In some embodiments, the CRISPR gene-editing system for the treatment of low back pain comprises one or more sgRNAs targeting exon 13 of cIL1RAP. In some embodiments, the CRISPR gene-editing system for the treatment of low back pain comprises one or more sgRNAs targeting exon 14 of cIL1RAP. In some embodiments, the CRISPR gene-editing system for the treatment of low back pain comprises one or more sgRNAs targeting exon 15 of cIL1RAP. In some embodiments, the CRISPR gene-editing system for the treatment of low back pain comprises one or more sgRNAs targeting exon 16 of cIL1RAP. In some embodiments, the CRISPR gene-editing system for the treatment of low back pain comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eIL1RAP. In some embodiments, the CRISPR gene-editing system for the treatment of low back pain comprises one or more sgRNAs targeting exon 2 of eIL1RAP. In some embodiments, the CRISPR gene-editing system for the treatment of low back pain comprises one or more sgRNAs targeting exon 3 of eIL1RAP. In some embodiments, the CRISPR gene-editing system for the treatment of low back pain comprises one or more sgRNAs targeting exon 4 of eIL1RAP. In some embodiments, the CRISPR gene-editing system for the treatment of low back pain comprises one or more sgRNAs targeting exon 5 of eIL1RAP. In some embodiments, the CRISPR gene-editing system for the treatment of low back pain comprises one or more sgRNAs targeting exon 6 of eIL1RAP. In some embodiments, the CRISPR gene-editing system for the treatment of low back pain comprises one or more sgRNAs targeting exon 7 of eIL1RAP. In some embodiments, the CRISPR gene-editing system for the treatment of low back pain comprises one or more sgRNAs targeting exon 8 of eIL1RAP. In some embodiments, the CRISPR gene-editing system for the treatment of low back pain comprises one or more sgRNAs targeting exon 9 of eIL1RAP. In some embodiments, the CRISPR gene-editing system for the treatment of low back pain comprises one or more sgRNAs targeting exon 10 of eIL1RAP. In some embodiments, the CRISPR gene-editing system for the treatment of low back pain comprises one or more sgRNAs targeting exon 11 of eIL1RAP. In some embodiments, the CRISPR gene-editing system for the treatment of low back pain comprises one or more sgRNAs targeting exon 12 of eIL1RAP. In some embodiments, the CRISPR gene-editing system for the treatment of low back pain comprises one or more sgRNAs targeting exon 13 of eIL1RAP. In some embodiments, the CRISPR gene-editing system for the treatment of low back pain comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fIL1RAP. In some embodiments, the CRISPR gene-editing system for the treatment of low back pain comprises one or more sgRNAs targeting exon 2 of fIL1RAP. In some embodiments, the CRISPR gene-editing system for the treatment of low back pain comprises one or more sgRNAs targeting exon 3 of fIL1RAP. In some embodiments, the CRISPR gene-editing system for the treatment of low back pain comprises one or more sgRNAs targeting exon 4 of fIL1RAP. In some embodiments, the CRISPR gene-editing system for the treatment of low back pain comprises one or more sgRNAs targeting exon 5 of fIL1RAP. In some embodiments, the CRISPR gene-editing system for the treatment of low back pain comprises one or more sgRNAs targeting exon 6 of fIL1RAP. In some embodiments, the CRISPR gene-editing system for the treatment of low back pain comprises one or more sgRNAs targeting exon 7 of fIL1RAP. In some embodiments, the CRISPR gene-editing system for the treatment of low back pain comprises one or more sgRNAs targeting exon 8 of fIL1RAP. In some embodiments, the CRISPR gene-editing system for the treatment of low back pain comprises one or more sgRNAs targeting exon 9 of fIL1RAP. In some embodiments, the CRISPR gene-editing system for the treatment of low back pain comprises one or more sgRNAs targeting exon 10 of fIL1RAP. In some embodiments, the CRISPR gene-editing system for the treatment of low back pain comprises one or more sgRNAs targeting exon 11 of fIL1RAP. In some embodiments, the CRISPR gene-editing system for the treatment of low back pain comprises one or more sgRNAs targeting exon 12 of fIL1RAP. In some embodiments, the CRISPR gene-editing system for the treatment of low back pain comprises one or more sgRNAs targeting exon 13 of fIL1RAP. In some embodiments, the CRISPR gene-editing system for the treatment of low back pain comprises one or more sgRNAs targeting exon 14 of fIL1RAP. In some embodiments, the CRISPR gene-editing system for the treatment of low back pain comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TGFBR1. In some embodiments, the CRISPR gene-editing system for the treatment of low back pain comprises one or more sgRNAs targeting hTGFBR1. In some embodiments, the CRISPR gene-editing system for the treatment of low back pain comprises one or more sgRNAs targeting cTGFBR1. In some embodiments, the CRISPR gene-editing system for the treatment of low back pain comprises one or more sgRNAs targeting e TGFBR1. In some embodiments, the CRISPR gene-editing system for the treatment of low back pain comprises one or more sgRNAs targeting fTGFBR1. In some embodiments, the CRISPR gene-editing system for the treatment of low back pain comprising one or more sgRNAs targeting an exon of TGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTGFBR1. In some embodiments, the CRISPR gene-editing system for the treatment of low back pain comprises one or more sgRNAs targeting exon 2 of hTGFBR1. In some embodiments, the CRISPR gene-editing system for the treatment of low back pain comprises one or more sgRNAs targeting exon 3 of hTGFBR1. In some embodiments, the CRISPR gene-editing system for the treatment of low back pain comprises one or more sgRNAs targeting exon 4 of hTGFBR1. In some embodiments, the CRISPR gene-editing system for the treatment of low back pain comprises one or more sgRNAs targeting exon 5 of hTGFBR1. In some embodiments, the CRISPR gene-editing system for the treatment of low back pain comprises one or more sgRNAs targeting exon 6 of hTGFBR1. In some embodiments, the CRISPR gene-editing system for the treatment of low back pain comprises one or more sgRNAs targeting exon 7 of hTGFBR1. In some embodiments, the CRISPR gene-editing system for the treatment of low back pain comprises one or more sgRNAs targeting exon 8 of hTGFBR1. In some embodiments, the CRISPR gene-editing system for the treatment of low back pain comprises one or more sgRNAs targeting exon 9 of hTGFBR1. In some embodiments, the CRISPR gene-editing system for the treatment of low back pain comprises one or more sgRNAs targeting exon 10 of hTGFBR1. In some embodiments, the CRISPR gene-editing system for the treatment of low back pain comprises one or more sgRNAs targeting exon 11 of hTGFBR1. In some embodiments, the CRISPR gene-editing system for the treatment of low back pain comprising one or more sgRNAs targeting an exon of hTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTGFBR1. In some embodiments, the CRISPR gene-editing system for the treatment of low back pain comprises one or more sgRNAs targeting exon 2 of cTGFBR1. In some embodiments, the CRISPR gene-editing system for the treatment of low back pain comprises one or more sgRNAs targeting exon 3 of cTGFBR1. In some embodiments, the CRISPR gene-editing system for the treatment of low back pain comprises one or more sgRNAs targeting exon 4 of cTGFBR1. In some embodiments, the CRISPR gene-editing system for the treatment of low back pain comprises one or more sgRNAs targeting exon 5 of cTGFBR1. In some embodiments, the CRISPR gene-editing system for the treatment of low back pain comprises one or more sgRNAs targeting exon 6 of cTGFBR1. In some embodiments, the CRISPR gene-editing system for the treatment of low back pain comprises one or more sgRNAs targeting exon 7 of cTGFBR1. In some embodiments, the CRISPR gene-editing system for the treatment of low back pain comprises one or more sgRNAs targeting exon 8 of cTGFBR1. In some embodiments, the CRISPR gene-editing system for the treatment of low back pain comprises one or more sgRNAs targeting exon 9 of cTGFBR1. In some embodiments, the CRISPR gene-editing system for the treatment of low back pain comprising one or more sgRNAs targeting an exon of cTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTGFBR1. In some embodiments, the CRISPR gene-editing system for the treatment of low back pain comprises one or more sgRNAs targeting exon 2 of eTGFBR1. In some embodiments, the CRISPR gene-editing system for the treatment of low back pain comprises one or more sgRNAs targeting exon 3 of eTGFBR1. In some embodiments, the CRISPR gene-editing system for the treatment of low back pain comprises one or more sgRNAs targeting exon 4 of eTGFBR1. In some embodiments, the CRISPR gene-editing system for the treatment of low back pain comprises one or more sgRNAs targeting exon 5 of eTGFBR1. In some embodiments, the CRISPR gene-editing system for the treatment of low back pain comprises one or more sgRNAs targeting exon 6 of eTGFBR1. In some embodiments, the CRISPR gene-editing system for the treatment of low back pain comprises one or more sgRNAs targeting exon 7 of eTGFBR1. In some embodiments, the CRISPR gene-editing system for the treatment of low back pain comprises one or more sgRNAs targeting exon 8 of eTGFBR1. In some embodiments, the CRISPR gene-editing system for the treatment of low back pain comprises one or more sgRNAs targeting exon 9 of eTGFBR1. In some embodiments, the CRISPR gene-editing system for the treatment of low back pain comprises one or more sgRNAs targeting exon 10 of eTGFBR1. In some embodiments, the CRISPR gene-editing system for the treatment of low back pain comprises one or more sgRNAs targeting exon 11 of eTGFBR1. In some embodiments, the CRISPR gene-editing system for the treatment of low back pain comprises one or more sgRNAs targeting exon 12 of eTGFBR1. In some embodiments, the CRISPR gene-editing system for the treatment of low back pain comprising one or more sgRNAs targeting an exon of eTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTGFBR1. In some embodiments, the CRISPR gene-editing system for the treatment of low back pain comprises one or more sgRNAs targeting exon 2 of fTGFBR1. In some embodiments, the CRISPR gene-editing system for the treatment of low back pain comprises one or more sgRNAs targeting exon 3 of fTGFBR1. In some embodiments, the CRISPR gene-editing system for the treatment of low back pain comprises one or more sgRNAs targeting exon 4 of fTGFBR1. In some embodiments, the CRISPR gene-editing system for the treatment of low back pain comprises one or more sgRNAs targeting exon 5 of fTGFBR1. In some embodiments, the CRISPR gene-editing system for the treatment of low back pain comprises one or more sgRNAs targeting exon 6 of fTGFBR1. In some embodiments, the CRISPR gene-editing system for the treatment of low back pain comprises one or more sgRNAs targeting exon 7 of fTGFBR1. In some embodiments, the CRISPR gene-editing system for the treatment of low back pain comprises one or more sgRNAs targeting exon 8 of fTGFBR1. In some embodiments, the CRISPR gene-editing system for the treatment of low back pain comprises one or more sgRNAs targeting exon 9 of fTGFBR1. In some embodiments, the CRISPR gene-editing system for the treatment of low back pain comprises one or more sgRNAs targeting exon 10 of fTGFBR1. In some embodiments, the CRISPR gene-editing system for the treatment of low back pain comprises one or more sgRNAs targeting exon 11 of fTGFBR1. In some embodiments, the CRISPR gene-editing system for the treatment of low back pain comprising one or more sgRNAs targeting an exon of fTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TGFBR2. In some embodiments, the CRISPR gene-editing system for the treatment of low back pain comprises one or more sgRNAs targeting hTGFBR2. In some embodiments, the CRISPR gene-editing system for the treatment of low back pain comprises one or more sgRNAs targeting cTGFBR2. In some embodiments, the CRISPR gene-editing system for the treatment of low back pain comprises one or more sgRNAs targeting eTGFBR2. In some embodiments, the CRISPR gene-editing system for the treatment of low back pain comprises one or more sgRNAs targeting fTGFBR2. In some embodiments, the CRISPR gene-editing system for the treatment of low back pain comprising one or more sgRNAs targeting an exon of TGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTGFBR2. In some embodiments, the CRISPR gene-editing system for the treatment of low back pain comprises one or more sgRNAs targeting exon 2 of hTGFBR2. In some embodiments, the CRISPR gene-editing system for the treatment of low back pain comprises one or more sgRNAs targeting exon 3 of hTGFBR2. In some embodiments, the CRISPR gene-editing system for the treatment of low back pain comprises one or more sgRNAs targeting exon 4 of hTGFBR2. In some embodiments, the CRISPR gene-editing system for the treatment of low back pain comprises one or more sgRNAs targeting exon 5 of hTGFBR2. In some embodiments, the CRISPR gene-editing system for the treatment of low back pain comprises one or more sgRNAs targeting exon 6 of hTGFBR2. In some embodiments, the CRISPR gene-editing system for the treatment of low back pain comprises one or more sgRNAs targeting exon 7 of hTGFBR2. In some embodiments, the CRISPR gene-editing system for the treatment of low back pain comprises one or more sgRNAs targeting exon 8 of hTGFBR2. In some embodiments, the CRISPR gene-editing system for the treatment of low back pain comprises one or more sgRNAs targeting exon 9 of hTGFBR2. In some embodiments, the CRISPR gene-editing system for the treatment of low back pain comprising one or more sgRNAs targeting an exon of hTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTGFBR2. In some embodiments, the CRISPR gene-editing system for the treatment of low back pain comprises one or more sgRNAs targeting exon 2 of cTGFBR2. In some embodiments, the CRISPR gene-editing system for the treatment of low back pain comprises one or more sgRNAs targeting exon 3 of cTGFBR2. In some embodiments, the CRISPR gene-editing system for the treatment of low back pain comprises one or more sgRNAs targeting exon 4 of cTGFBR2. In some embodiments, the CRISPR gene-editing system for the treatment of low back pain comprises one or more sgRNAs targeting exon 5 of cTGFBR2. In some embodiments, the CRISPR gene-editing system for the treatment of low back pain comprises one or more sgRNAs targeting exon 6 of cTGFBR2. In some embodiments, the CRISPR gene-editing system for the treatment of low back pain comprises one or more sgRNAs targeting exon 7 of cTGFBR2. In some embodiments, the CRISPR gene-editing system for the treatment of low back pain comprises one or more sgRNAs targeting exon 8 of cTGFBR2. In some embodiments, the CRISPR gene-editing system for the treatment of low back pain comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTGFBR2. In some embodiments, the CRISPR gene-editing system for the treatment of low back pain comprises one or more sgRNAs targeting exon 2 of eTGFBR2. In some embodiments, the CRISPR gene-editing system for the treatment of low back pain comprises one or more sgRNAs targeting exon 3 of eTGFBR2. In some embodiments, the CRISPR gene-editing system for the treatment of low back pain comprises one or more sgRNAs targeting exon 4 of eTGFBR2. In some embodiments, the CRISPR gene-editing system for the treatment of low back pain comprises one or more sgRNAs targeting exon 5 of eTGFBR2. In some embodiments, the CRISPR gene-editing system for the treatment of low back pain comprises one or more sgRNAs targeting exon 6 of eTGFBR2. In some embodiments, the CRISPR gene-editing system for the treatment of low back pain comprises one or more sgRNAs targeting exon 7 of eTGFBR2. In some embodiments, the CRISPR gene-editing system for the treatment of low back pain comprises one or more sgRNAs targeting exon 8 of eTGFBR2. In some embodiments, the CRISPR gene-editing system for the treatment of low back pain comprises one or more sgRNAs targeting exon 9 of eTGFBR2. In some embodiments, the CRISPR gene-editing system for the treatment of low back pain comprises one or more sgRNAs targeting exon 10 of eTGFBR2. In some embodiments, the CRISPR gene-editing system for the treatment of low back pain comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTGFBR2. In some embodiments, the CRISPR gene-editing system for the treatment of low back pain comprises one or more sgRNAs targeting exon 2 of fTGFBR2. In some embodiments, the CRISPR gene-editing system for the treatment of low back pain comprises one or more sgRNAs targeting exon 3 of fTGFBR2. In some embodiments, the CRISPR gene-editing system for the treatment of low back pain comprises one or more sgRNAs targeting exon 4 of fTGFBR2. In some embodiments, the CRISPR gene-editing system for the treatment of low back pain comprises one or more sgRNAs targeting exon 5 of fTGFBR2. In some embodiments, the CRISPR gene-editing system for the treatment of low back pain comprises one or more sgRNAs targeting exon 6 of fTGFBR2. In some embodiments, the CRISPR gene-editing system for the treatment of low back pain comprises one or more sgRNAs targeting exon 7 of fTGFBR2. In some embodiments, the CRISPR gene-editing system for the treatment of low back pain comprises one or more sgRNAs targeting exon 8 of fTGFBR2. In some embodiments, the CRISPR gene-editing system for the treatment of low back pain comprising one or more sgRNAs targeting an exon of fTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting IL6R. In some embodiments, the CRISPR gene-editing system for the treatment of low back pain comprises one or more sgRNAs targeting hIL6R. In some embodiments, the CRISPR gene-editing system for the treatment of low back pain comprises one or more sgRNAs targeting cIL6R. In some embodiments, the CRISPR gene-editing system for the treatment of low back pain comprises one or more sgRNAs targeting eIL6R. In some embodiments, the CRISPR gene-editing system for the treatment of low back pain comprises one or more sgRNAs targeting fIL6R. In some embodiments, the CRISPR gene-editing system for the treatment of low back pain comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hIL6R. In some embodiments, the CRISPR gene-editing system for the treatment of low back pain comprises one or more sgRNAs targeting exon 2 of hIL6R. In some embodiments, the CRISPR gene-editing system for the treatment of low back pain comprises one or more sgRNAs targeting exon 3 of hIL6R. In some embodiments, the CRISPR gene-editing system for the treatment of low back pain comprises one or more sgRNAs targeting exon 4 of hIL6R. In some embodiments, the CRISPR gene-editing system for the treatment of low back pain comprises one or more sgRNAs targeting exon 5 of hIL6R. In some embodiments, the CRISPR gene-editing system for the treatment of low back pain comprises one or more sgRNAs targeting exon 6 of hIL6R. In some embodiments, the CRISPR gene-editing system for the treatment of low back pain comprises one or more sgRNAs targeting exon 7 of hIL6R. In some embodiments, the CRISPR gene-editing system for the treatment of low back pain comprises one or more sgRNAs targeting exon 8 of hIL6R. In some embodiments, the CRISPR gene-editing system for the treatment of low back pain comprises one or more sgRNAs targeting exon 9 of hIL6R. In some embodiments, the CRISPR gene-editing system for the treatment of low back pain comprises one or more sgRNAs targeting exon 10 of hIL6R. In some embodiments, the CRISPR gene-editing system for the treatment of low back pain comprises one or more sgRNAs targeting exon 11 of hIL6R In some embodiments, the CRISPR gene-editing system for the treatment of low back pain comprises one or more sgRNAs targeting exon 12 of hIL6R. In some embodiments, the CRISPR gene-editing system for the treatment of low back pain comprises one or more sgRNAs targeting exon 13 of hIL6R. In some embodiments, the CRISPR gene-editing system for the treatment of low back pain comprises one or more sgRNAs targeting exon 14 of hIL6R. In some embodiments, the CRISPR gene-editing system for the treatment of low back pain comprises one or more sgRNAs targeting exon 15 of hIL6R. In some embodiments, the CRISPR gene-editing system for the treatment of low back pain comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cIL6R. In some embodiments, the CRISPR gene-editing system for the treatment of low back pain comprises one or more sgRNAs targeting exon 2 of cIL6R. In some embodiments, the CRISPR gene-editing system for the treatment of low back pain comprises one or more sgRNAs targeting exon 3 of cIL6R. In some embodiments, the CRISPR gene-editing system for the treatment of low back pain comprises one or more sgRNAs targeting exon 4 of cIL6R. In some embodiments, the CRISPR gene-editing system for the treatment of low back pain comprises one or more sgRNAs targeting exon 5 of cIL6R. In some embodiments, the CRISPR gene-editing system for the treatment of low back pain comprises one or more sgRNAs targeting exon 6 of cIL6R. In some embodiments, the CRISPR gene-editing system for the treatment of low back pain comprises one or more sgRNAs targeting exon 7 of cIL6R. In some embodiments, the CRISPR gene-editing system for the treatment of low back pain comprises one or more sgRNAs targeting exon 8 of cIL6R. In some embodiments, the CRISPR gene-editing system for the treatment of low back pain comprises one or more sgRNAs targeting exon 9 of cIL6R. In some embodiments, the CRISPR gene-editing system for the treatment of low back pain comprises one or more sgRNAs targeting exon 10 of cIL6R. In some embodiments, the CRISPR gene-editing system for the treatment of low back pain comprises one or more sgRNAs targeting exon 11 of cIL6R. In some embodiments, the CRISPR gene-editing system for the treatment of low back pain comprises one or more sgRNAs targeting exon 12 of cIL6R. In some embodiments, the CRISPR gene-editing system for the treatment of low back pain comprises one or more sgRNAs targeting exon 13 of cIL6R. In some embodiments, the CRISPR gene-editing system for the treatment of low back pain comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eIL6R. In some embodiments, the CRISPR gene-editing system for the treatment of low back pain comprises one or more sgRNAs targeting exon 2 of eIL6R. In some embodiments, the CRISPR gene-editing system for the treatment of low back pain comprises one or more sgRNAs targeting exon 3 of eIL6R. In some embodiments, the CRISPR gene-editing system for the treatment of low back pain comprises one or more sgRNAs targeting exon 4 of eIL6R. In some embodiments, the CRISPR gene-editing system for the treatment of low back pain comprises one or more sgRNAs targeting exon 5 of eIL6R. In some embodiments, the CRISPR gene-editing system for the treatment of low back pain comprises one or more sgRNAs targeting exon 6 of eIL6R. In some embodiments, the CRISPR gene-editing system for the treatment of low back pain comprises one or more sgRNAs targeting exon 7 of eIL6R. In some embodiments, the CRISPR gene-editing system for the treatment of low back pain comprises one or more sgRNAs targeting exon 8 of eIL6R. In some embodiments, the CRISPR gene-editing system for the treatment of low back pain comprises one or more sgRNAs targeting exon 9 of eIL6R. In some embodiments, the CRISPR gene-editing system for the treatment of low back pain comprises one or more sgRNAs targeting exon 10 of eIL6R. In some embodiments, the CRISPR gene-editing system for the treatment of low back pain comprises one or more sgRNAs targeting exon 11 of eIL6R. In some embodiments, the CRISPR gene-editing system for the treatment of low back pain comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fIL6R. In some embodiments, the CRISPR gene-editing system for the treatment of low back pain comprises one or more sgRNAs targeting exon 2 of fIL6R. In some embodiments, the CRISPR gene-editing system for the treatment of low back pain comprises one or more sgRNAs targeting exon 3 of fIL6R. In some embodiments, the CRISPR gene-editing system for the treatment of low back pain comprises one or more sgRNAs targeting exon 4 of fIL6R. In some embodiments, the CRISPR gene-editing system for the treatment of low back pain comprises one or more sgRNAs targeting exon 5 of fIL6R. In some embodiments, the CRISPR gene-editing system for the treatment of low back pain comprises one or more sgRNAs targeting exon 6 of fIL6R. In some embodiments, the CRISPR gene-editing system for the treatment of low back pain comprises one or more sgRNAs targeting exon 7 of fIL6R. In some embodiments, the CRISPR gene-editing system for the treatment of low back pain comprises one or more sgRNAs targeting exon 8 of fIL6R. In some embodiments, the CRISPR gene-editing system for the treatment of low back pain comprises one or more sgRNAs targeting exon 9 of fIL6R. In some embodiments, the CRISPR gene-editing system for the treatment of low back pain comprises one or more sgRNAs targeting exon 10 of fIL6R. In some embodiments, the CRISPR gene-editing system for the treatment of low back pain comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting IL6ST. In some embodiments, the CRISPR gene-editing system for the treatment of low back pain comprises one or more sgRNAs targeting hIL6ST. In some embodiments, the CRISPR gene-editing system for the treatment of low back pain comprises one or more sgRNAs targeting cIL6ST. In some embodiments, the CRISPR gene-editing system for the treatment of low back pain comprises one or more sgRNAs targeting eIL6ST. In some embodiments, the CRISPR gene-editing system for the treatment of low back pain comprises one or more sgRNAs targeting fIL6ST. In some embodiments, the CRISPR gene-editing system for the treatment of low back pain comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hIL6ST. In some embodiments, the CRISPR gene-editing system for the treatment of low back pain comprises one or more sgRNAs targeting exon 2 of hIL6ST. In some embodiments, the CRISPR gene-editing system for the treatment of low back pain comprises one or more sgRNAs targeting exon 3 of hIL6ST. In some embodiments, the CRISPR gene-editing system for the treatment of low back pain comprises one or more sgRNAs targeting exon 4 of hIL6ST. In some embodiments, the CRISPR gene-editing system for the treatment of low back pain comprises one or more sgRNAs targeting exon 5 of hIL6ST. In some embodiments, the CRISPR gene-editing system for the treatment of low back pain comprises one or more sgRNAs targeting exon 6 of hIL6ST. In some embodiments, the CRISPR gene-editing system for the treatment of low back pain comprises one or more sgRNAs targeting exon 7 of hIL6ST. In some embodiments, the CRISPR gene-editing system for the treatment of low back pain comprises one or more sgRNAs targeting exon 8 of hIL6ST. In some embodiments, the CRISPR gene-editing system for the treatment of low back pain comprises one or more sgRNAs targeting exon 9 of hIL6ST. In some embodiments, the CRISPR gene-editing system for the treatment of low back pain comprises one or more sgRNAs targeting exon 10 of hIL6ST. In some embodiments, the CRISPR gene-editing system for the treatment of low back pain comprises one or more sgRNAs targeting exon 11 of hIL6ST. In some embodiments, the CRISPR gene-editing system for the treatment of low back pain comprises one or more sgRNAs targeting exon 12 of hIL6ST. In some embodiments, the CRISPR gene-editing system for the treatment of low back pain comprises one or more sgRNAs targeting exon 13 of hIL6ST. In some embodiments, the CRISPR gene-editing system for the treatment of low back pain comprises one or more sgRNAs targeting exon 14 of hIL6ST. In some embodiments, the CRISPR gene-editing system for the treatment of low back pain comprises one or more sgRNAs targeting exon 15 of hIL6ST. In some embodiments, the CRISPR gene-editing system for the treatment of low back pain comprises one or more sgRNAs targeting exon 16 of hIL6ST. In some embodiments, the CRISPR gene-editing system for the treatment of low back pain comprises one or more sgRNAs targeting exon 17 of hIL6ST. In some embodiments, the CRISPR gene-editing system for the treatment of low back pain comprises one or more sgRNAs targeting exon 18 of hIL6ST. In some embodiments, the CRISPR gene-editing system for the treatment of low back pain comprises one or more sgRNAs targeting exon 19 of hIL6ST. In some embodiments, the CRISPR gene-editing system for the treatment of low back pain comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cIL6ST. In some embodiments, the CRISPR gene-editing system for the treatment of low back pain comprises one or more sgRNAs targeting exon 2 of cIL6ST. In some embodiments, the CRISPR gene-editing system for the treatment of low back pain comprises one or more sgRNAs targeting exon 3 of cIL6ST. In some embodiments, the CRISPR gene-editing system for the treatment of low back pain comprises one or more sgRNAs targeting exon 4 of cIL6ST. In some embodiments, the CRISPR gene-editing system for the treatment of low back pain comprises one or more sgRNAs targeting exon 5 of cIL6ST. In some embodiments, the CRISPR gene-editing system for the treatment of low back pain comprises one or more sgRNAs targeting exon 6 of cIL6ST. In some embodiments, the CRISPR gene-editing system for the treatment of low back pain comprises one or more sgRNAs targeting exon 7 of cIL6ST. In some embodiments, the CRISPR gene-editing system for the treatment of low back pain comprises one or more sgRNAs targeting exon 8 of cIL6ST. In some embodiments, the CRISPR gene-editing system for the treatment of low back pain comprises one or more sgRNAs targeting exon 9 of cIL6ST. In some embodiments, the CRISPR gene-editing system for the treatment of low back pain comprises one or more sgRNAs targeting exon 10 of cIL6ST. In some embodiments, the CRISPR gene-editing system for the treatment of low back pain comprises one or more sgRNAs targeting exon 11 of cIL6ST In some embodiments, the CRISPR gene-editing system for the treatment of low back pain comprises one or more sgRNAs targeting exon 12 of cIL6ST. In some embodiments, the CRISPR gene-editing system for the treatment of low back pain comprises one or more sgRNAs targeting exon 13 of cIL6ST. In some embodiments, the CRISPR gene-editing system for the treatment of low back pain comprises one or more sgRNAs targeting exon 14 of cIL6ST. In some embodiments, the CRISPR gene-editing system for the treatment of low back pain comprises one or more sgRNAs targeting exon 15 of cIL6ST. In some embodiments, the CRISPR gene-editing system for the treatment of low back pain comprises one or more sgRNAs targeting exon 16 of cIL6ST. In some embodiments, the CRISPR gene-editing system for the treatment of low back pain comprises one or more sgRNAs targeting exon 17 of cIL6ST. In some embodiments, the CRISPR gene-editing system for the treatment of low back pain comprises one or more sgRNAs targeting exon 18 of cIL6ST. In some embodiments, the CRISPR gene-editing system for the treatment of low back pain comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eIL6ST. In some embodiments, the CRISPR gene-editing system for the treatment of low back pain comprises one or more sgRNAs targeting exon 2 of eIL6ST. In some embodiments, the CRISPR gene-editing system for the treatment of low back pain comprises one or more sgRNAs targeting exon 3 of eIL6ST. In some embodiments, the CRISPR gene-editing system for the treatment of low back pain comprises one or more sgRNAs targeting exon 4 of eIL6ST. In some embodiments, the CRISPR gene-editing system for the treatment of low back pain comprises one or more sgRNAs targeting exon 5 of eIL6ST. In some embodiments, the CRISPR gene-editing system for the treatment of low back pain comprises one or more sgRNAs targeting exon 6 of eIL6ST. In some embodiments, the CRISPR gene-editing system for the treatment of low back pain comprises one or more sgRNAs targeting exon 7 of eIL6ST. In some embodiments, the CRISPR gene-editing system for the treatment of low back pain comprises one or more sgRNAs targeting exon 8 of eIL6ST. In some embodiments, the CRISPR gene-editing system for the treatment of low back pain comprises one or more sgRNAs targeting exon 9 of eIL6ST. In some embodiments, the CRISPR gene-editing system for the treatment of low back pain comprises one or more sgRNAs targeting exon 10 of eIL6ST. In some embodiments, the CRISPR gene-editing system for the treatment of low back pain comprises one or more sgRNAs targeting exon 11 of eIL6ST In some embodiments, the CRISPR gene-editing system for the treatment of low back pain comprises one or more sgRNAs targeting exon 12 of eIL6ST. In some embodiments, the CRISPR gene-editing system for the treatment of low back pain comprises one or more sgRNAs targeting exon 13 of eIL6ST. In some embodiments, the CRISPR gene-editing system for the treatment of low back pain comprises one or more sgRNAs targeting exon 14 of eIL6ST. In some embodiments, the CRISPR gene-editing system for the treatment of low back pain comprises one or more sgRNAs targeting exon 15 of eIL6ST. In some embodiments, the CRISPR gene-editing system for the treatment of low back pain comprises one or more sgRNAs targeting exon 16 of eIL6ST. In some embodiments, the CRISPR gene-editing system for the treatment of low back pain comprises one or more sgRNAs targeting exon 17 of eIL6ST. In some embodiments, the CRISPR gene-editing system for the treatment of low back pain comprises one or more sgRNAs targeting exon 18 of eIL6ST. In some embodiments, the CRISPR gene-editing system for the treatment of low back pain comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fIL6ST. In some embodiments, the CRISPR gene-editing system for the treatment of low back pain comprises one or more sgRNAs targeting exon 2 of fIL6ST. In some embodiments, the CRISPR gene-editing system for the treatment of low back pain comprises one or more sgRNAs targeting exon 3 of fIL6ST. In some embodiments, the CRISPR gene-editing system for the treatment of low back pain comprises one or more sgRNAs targeting exon 4 of fIL6ST. In some embodiments, the CRISPR gene-editing system for the treatment of low back pain comprises one or more sgRNAs targeting exon 5 of fIL6ST. In some embodiments, the CRISPR gene-editing system for the treatment of low back pain comprises one or more sgRNAs targeting exon 6 of fIL6ST. In some embodiments, the CRISPR gene-editing system for the treatment of low back pain comprises one or more sgRNAs targeting exon 7 of fIL6ST. In some embodiments, the CRISPR gene-editing system for the treatment of low back pain comprises one or more sgRNAs targeting exon 8 of fIL6ST. In some embodiments, the CRISPR gene-editing system for the treatment of low back pain comprises one or more sgRNAs targeting exon 9 of fIL6ST. In some embodiments, the CRISPR gene-editing system for the treatment of low back pain comprises one or more sgRNAs targeting exon 10 of fIL6ST. In some embodiments, the CRISPR gene-editing system for the treatment of low back pain comprises one or more sgRNAs targeting exon 11 of fIL6ST In some embodiments, the CRISPR gene-editing system for the treatment of low back pain comprises one or more sgRNAs targeting exon 12 of fIL6ST. In some embodiments, the CRISPR gene-editing system for the treatment of low back pain comprises one or more sgRNAs targeting exon 13 of fIL6ST. In some embodiments, the CRISPR gene-editing system for the treatment of low back pain comprises one or more sgRNAs targeting exon 14 of fIL6ST. In some embodiments, the CRISPR gene-editing system for the treatment of low back pain comprises one or more sgRNAs targeting exon 15 of fIL6ST. In some embodiments, the CRISPR gene-editing system for the treatment of low back pain comprises one or more sgRNAs targeting exon 16 of fIL6ST. In some embodiments, the CRISPR gene-editing system for the treatment of low back pain comprises one or more sgRNAs targeting exon 17 of fIL6ST. In some embodiments, the CRISPR gene-editing system for the treatment of low back pain comprises one or more sgRNAs targeting exon 18 of fIL6ST. In some embodiments, the CRISPR gene-editing system for the treatment of low back pain comprises one or more sgRNAs targeting exon 19 of fIL6ST. In some embodiments, the CRISPR gene-editing system for the treatment of low back pain comprises one or more sgRNAs targeting exon 20 of fIL6ST. In some embodiments, the CRISPR gene-editing system for the treatment of low back pain comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TNFRSF1A. In some embodiments, the CRISPR gene-editing system for the treatment of low back pain comprises one or more sgRNAs targeting hTNFRSF1A. In some embodiments, the CRISPR gene-editing system for the treatment of low back pain comprises one or more sgRNAs targeting cTNFRSF1A. In some embodiments, the CRISPR gene-editing system for the treatment of low back pain comprises one or more sgRNAs targeting eTNFRSF1A. In some embodiments, the CRISPR gene-editing system for the treatment of low back pain comprises one or more sgRNAs targeting fTNFRSF1A. In some embodiments, the CRISPR gene-editing system for the treatment of low back pain comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTNFRSF1A. In some embodiments, the CRISPR gene-editing system for the treatment of low back pain comprises one or more sgRNAs targeting exon 2 of hTNFRSF1A. In some embodiments, the CRISPR gene-editing system for the treatment of low back pain comprises one or more sgRNAs targeting exon 3 of hTNFRSF1A. In some embodiments, the CRISPR gene-editing system for the treatment of low back pain comprises one or more sgRNAs targeting exon 4 of hTNFRSF1A. In some embodiments, the CRISPR gene-editing system for the treatment of low back pain comprises one or more sgRNAs targeting exon 5 of hTNFRSF1A. In some embodiments, the CRISPR gene-editing system for the treatment of low back pain comprises one or more sgRNAs targeting exon 6 of hTNFRSF1A. In some embodiments, the CRISPR gene-editing system for the treatment of low back pain comprises one or more sgRNAs targeting exon 7 of hTNFRSF1A. In some embodiments, the CRISPR gene-editing system for the treatment of low back pain comprises one or more sgRNAs targeting exon 8 of hTNFRSF1A. In some embodiments, the CRISPR gene-editing system for the treatment of low back pain comprises one or more sgRNAs targeting exon 9 of hTNFRSF1A. In some embodiments, the CRISPR gene-editing system for the treatment of low back pain comprises one or more sgRNAs targeting exon 10 of hTNFRSF1A. In some embodiments, the CRISPR gene-editing system for the treatment of low back pain comprises one or more sgRNAs targeting exon 11 of hTNFRSF1A. In some embodiments, the CRISPR gene-editing system for the treatment of low back pain comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTNFRSF1A. In some embodiments, the CRISPR gene-editing system for the treatment of low back pain comprises one or more sgRNAs targeting exon 2 of cTNFRSF1A. In some embodiments, the CRISPR gene-editing system for the treatment of low back pain comprises one or more sgRNAs targeting exon 3 of cTNFRSF1A. In some embodiments, the CRISPR gene-editing system for the treatment of low back pain comprises one or more sgRNAs targeting exon 4 of cTNFRSF1A. In some embodiments, the CRISPR gene-editing system for the treatment of low back pain comprises one or more sgRNAs targeting exon 5 of cTNFRSF1A. In some embodiments, the CRISPR gene-editing system for the treatment of low back pain comprises one or more sgRNAs targeting exon 6 of cTNFRSF1A. In some embodiments, the CRISPR gene-editing system for the treatment of low back pain comprises one or more sgRNAs targeting exon 7 of cTNFRSF1A. In some embodiments, the CRISPR gene-editing system for the treatment of low back pain comprises one or more sgRNAs targeting exon 8 of cTNFRSF1A. In some embodiments, the CRISPR gene-editing system for the treatment of low back pain comprises one or more sgRNAs targeting exon 9 of cTNFRSF1A. In some embodiments, the CRISPR gene-editing system for the treatment of low back pain comprises one or more sgRNAs targeting exon 10 of cTNFRSF1A. In some embodiments, the CRISPR gene-editing system for the treatment of low back pain comprises one or more sgRNAs targeting exon 11 of cTNFRSF1A. In some embodiments, the CRISPR gene-editing system for the treatment of low back pain comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTNFRSF1A. In some embodiments, the CRISPR gene-editing system for the treatment of low back pain comprises one or more sgRNAs targeting exon 2 of eTNFRSF1A. In some embodiments, the CRISPR gene-editing system for the treatment of low back pain comprises one or more sgRNAs targeting exon 3 of eTNFRSF1A. In some embodiments, the CRISPR gene-editing system for the treatment of low back pain comprises one or more sgRNAs targeting exon 4 of eTNFRSF1A. In some embodiments, the CRISPR gene-editing system for the treatment of low back pain comprises one or more sgRNAs targeting exon 5 of eTNFRSF1A. In some embodiments, the CRISPR gene-editing system for the treatment of low back pain comprises one or more sgRNAs targeting exon 6 of eTNFRSF1A. In some embodiments, the CRISPR gene-editing system for the treatment of low back pain comprises one or more sgRNAs targeting exon 7 of eTNFRSF1A. In some embodiments, the CRISPR gene-editing system for the treatment of low back pain comprises one or more sgRNAs targeting exon 8 of eTNFRSF1A. In some embodiments, the CRISPR gene-editing system for the treatment of low back pain comprises one or more sgRNAs targeting exon 9 of eTNFRSF1A. In some embodiments, the CRISPR gene-editing system for the treatment of low back pain comprises one or more sgRNAs targeting exon 10 of eTNFRSF1A. In some embodiments, the CRISPR gene-editing system for the treatment of low back pain comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTNFRSF1A. In some embodiments, the CRISPR gene-editing system for the treatment of low back pain comprises one or more sgRNAs targeting exon 2 of fTNFRSF1A. In some embodiments, the CRISPR gene-editing system for the treatment of low back pain comprises one or more sgRNAs targeting exon 3 of fTNFRSF1A. In some embodiments, the CRISPR gene-editing system for the treatment of low back pain comprises one or more sgRNAs targeting exon 4 of fTNFRSF1A. In some embodiments, the CRISPR gene-editing system for the treatment of low back pain comprises one or more sgRNAs targeting exon 5 of fTNFRSF1A. In some embodiments, the CRISPR gene-editing system for the treatment of low back pain comprises one or more sgRNAs targeting exon 6 of fTNFRSF1A. In some embodiments, the CRISPR gene-editing system for the treatment of low back pain comprises one or more sgRNAs targeting exon 7 of fTNFRSF1A. In some embodiments, the CRISPR gene-editing system for the treatment of low back pain comprises one or more sgRNAs targeting exon 8 of fTNFRSF1A. In some embodiments, the CRISPR gene-editing system for the treatment of low back pain comprises one or more sgRNAs targeting exon 9 of fTNFRSF1A. In some embodiments, the CRISPR gene-editing system for the treatment of low back pain comprises one or more sgRNAs targeting exon 10 of fTNFRSF1A. In some embodiments, the CRISPR gene-editing system for the treatment of low back pain comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TNFRSF1B. In some embodiments, the CRISPR gene-editing system for the treatment of low back pain comprises one or more sgRNAs targeting hTNFRSF1B. In some embodiments, the CRISPR gene-editing system for the treatment of low back pain comprises one or more sgRNAs targeting cTNFRSF1B. In some embodiments, the CRISPR gene-editing system for the treatment of low back pain comprises one or more sgRNAs targeting eTNFRSF1B. In some embodiments, the CRISPR gene-editing system for the treatment of low back pain comprises one or more sgRNAs targeting fTNFRSF1B. In some embodiments, the CRISPR gene-editing system for the treatment of low back pain comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTNFRSF1B. In some embodiments, the CRISPR gene-editing system for the treatment of low back pain comprises one or more sgRNAs targeting exon 2 of hTNFRSF1B. In some embodiments, the CRISPR gene-editing system for the treatment of low back pain comprises one or more sgRNAs targeting exon 3 of hTNFRSF1B. In some embodiments, the CRISPR gene-editing system for the treatment of low back pain comprises one or more sgRNAs targeting exon 4 of hTNFRSF1B. In some embodiments, the CRISPR gene-editing system for the treatment of low back pain comprises one or more sgRNAs targeting exon 5 of hTNFRSF1B. In some embodiments, the CRISPR gene-editing system for the treatment of low back pain comprises one or more sgRNAs targeting exon 6 of hTNFRSF1B. In some embodiments, the CRISPR gene-editing system for the treatment of low back pain comprises one or more sgRNAs targeting exon 7 of hTNFRSF1B. In some embodiments, the CRISPR gene-editing system for the treatment of low back pain comprises one or more sgRNAs targeting exon 8 of hTNFRSF1B. In some embodiments, the CRISPR gene-editing system for the treatment of low back pain comprises one or more sgRNAs targeting exon 9 of hTNFRSF1B. In some embodiments, the CRISPR gene-editing system for the treatment of low back pain comprises one or more sgRNAs targeting exon 10 of hTNFRSF1B. In some embodiments, the CRISPR gene-editing system for the treatment of low back pain comprises one or more sgRNAs targeting exon 11 of hTNFRSF1B In some embodiments, the CRISPR gene-editing system for the treatment of low back pain comprises one or more sgRNAs targeting exon 12 of hTNFRSF1B. In some embodiments, the CRISPR gene-editing system for the treatment of low back pain comprises one or more sgRNAs targeting exon 13 of hTNFRSF1B. In some embodiments, the CRISPR gene-editing system for the treatment of low back pain comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTNFRSF1B. In some embodiments, the CRISPR gene-editing system for the treatment of low back pain comprises one or more sgRNAs targeting exon 2 of cTNFRSF1B. In some embodiments, the CRISPR gene-editing system for the treatment of low back pain comprises one or more sgRNAs targeting exon 3 of cTNFRSF1B. In some embodiments, the CRISPR gene-editing system for the treatment of low back pain comprises one or more sgRNAs targeting exon 4 of cTNFRSF1B. In some embodiments, the CRISPR gene-editing system for the treatment of low back pain comprises one or more sgRNAs targeting exon 5 of cTNFRSF1B. In some embodiments, the CRISPR gene-editing system for the treatment of low back pain comprises one or more sgRNAs targeting exon 6 of cTNFRSF1B. In some embodiments, the CRISPR gene-editing system for the treatment of low back pain comprises one or more sgRNAs targeting exon 7 of cTNFRSF1B. In some embodiments, the CRISPR gene-editing system for the treatment of low back pain comprises one or more sgRNAs targeting exon 8 of cTNFRSF1B. In some embodiments, the CRISPR gene-editing system for the treatment of low back pain comprises one or more sgRNAs targeting exon 9 of cTNFRSF1B. In some embodiments, the CRISPR gene-editing system for the treatment of low back pain comprises one or more sgRNAs targeting exon 10 of cTNFRSF1B. In some embodiments, the CRISPR gene-editing system for the treatment of low back pain comprises one or more sgRNAs targeting exon 11 of cTNFRSF1B. In some embodiments, the CRISPR gene-editing system for the treatment of low back pain comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTNFRSF1B. In some embodiments, the CRISPR gene-editing system for the treatment of low back pain comprises one or more sgRNAs targeting exon 2 of eTNFRSF1B. In some embodiments, the CRISPR gene-editing system for the treatment of low back pain comprises one or more sgRNAs targeting exon 3 of eTNFRSF1B. In some embodiments, the CRISPR gene-editing system for the treatment of low back pain comprises one or more sgRNAs targeting exon 4 of eTNFRSF1B. In some embodiments, the CRISPR gene-editing system for the treatment of low back pain comprises one or more sgRNAs targeting exon 5 of eTNFRSF1B. In some embodiments, the CRISPR gene-editing system for the treatment of low back pain comprises one or more sgRNAs targeting exon 6 of eTNFRSF1B. In some embodiments, the CRISPR gene-editing system for the treatment of low back pain comprises one or more sgRNAs targeting exon 7 of eTNFRSF1B. In some embodiments, the CRISPR gene-editing system for the treatment of low back pain comprises one or more sgRNAs targeting exon 8 of eTNFRSF1B. In some embodiments, the CRISPR gene-editing system for the treatment of low back pain comprises one or more sgRNAs targeting exon 9 of eTNFRSF1B. In some embodiments, the CRISPR gene-editing system for the treatment of low back pain comprises one or more sgRNAs targeting exon 10 of eTNFRSF1B. In some embodiments, the CRISPR gene-editing system for the treatment of low back pain comprises one or more sgRNAs targeting exon 11 of eTNFRSF1B. In some embodiments, the CRISPR gene-editing system for the treatment of low back pain comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTNFRSF1B. In some embodiments, the CRISPR gene-editing system for the treatment of low back pain comprises one or more sgRNAs targeting exon 2 of fTNFRSF1B. In some embodiments, the CRISPR gene-editing system for the treatment of low back pain comprises one or more sgRNAs targeting exon 3 of fTNFRSF1B. In some embodiments, the CRISPR gene-editing system for the treatment of low back pain comprises one or more sgRNAs targeting exon 4 of fTNFRSF1B. In some embodiments, the CRISPR gene-editing system for the treatment of low back pain comprises one or more sgRNAs targeting exon 5 of fTNFRSF1B. In some embodiments, the CRISPR gene-editing system for the treatment of low back pain comprises one or more sgRNAs targeting exon 6 of fTNFRSF1B. In some embodiments, the CRISPR gene-editing system for the treatment of low back pain comprises one or more sgRNAs targeting exon 7 of fTNFRSF1B. In some embodiments, the CRISPR gene-editing system for the treatment of low back pain comprises one or more sgRNAs targeting exon 8 of fTNFRSF1B. In some embodiments, the CRISPR gene-editing system for the treatment of low back pain comprises one or more sgRNAs targeting exon 9 of fTNFRSF1B. In some embodiments, the CRISPR gene-editing system for the treatment of low back pain comprises one or more sgRNAs targeting exon 10 of fTNFRSF1B. In some embodiments, the CRISPR gene-editing system for the treatment of low back pain comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TNFRSF3. In some embodiments, the CRISPR gene-editing system for the treatment of low back pain comprises one or more sgRNAs targeting hTNFRSF3. In some embodiments, the CRISPR gene-editing system for the treatment of low back pain comprises one or more sgRNAs targeting cTNFRSF3. In some embodiments, the CRISPR gene-editing system for the treatment of low back pain comprises one or more sgRNAs targeting eTNFRSF3. In some embodiments, the CRISPR gene-editing system for the treatment of low back pain comprises one or more sgRNAs targeting fTNFRSF3. In some embodiments, the CRISPR gene-editing system for the treatment of low back pain comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTNFRSF3. In some embodiments, the CRISPR gene-editing system for the treatment of low back pain comprises one or more sgRNAs targeting exon 2 of hTNFRSF3. In some embodiments, the CRISPR gene-editing system for the treatment of low back pain comprises one or more sgRNAs targeting exon 3 of hTNFRSF3. In some embodiments, the CRISPR gene-editing system for the treatment of low back pain comprises one or more sgRNAs targeting exon 4 of hTNFRSF3. In some embodiments, the CRISPR gene-editing system for the treatment of low back pain comprises one or more sgRNAs targeting exon 5 of hTNFRSF3. In some embodiments, the CRISPR gene-editing system for the treatment of low back pain comprises one or more sgRNAs targeting exon 6 of hTNFRSF3. In some embodiments, the CRISPR gene-editing system for the treatment of low back pain comprises one or more sgRNAs targeting exon 7 of hTNFRSF3. In some embodiments, the CRISPR gene-editing system for the treatment of low back pain comprises one or more sgRNAs targeting exon 8 of hTNFRSF3. In some embodiments, the CRISPR gene-editing system for the treatment of low back pain comprises one or more sgRNAs targeting exon 9 of hTNFRSF3. In some embodiments, the CRISPR gene-editing system for the treatment of low back pain comprises one or more sgRNAs targeting exon 10 of hTNFRSF3. In some embodiments, the CRISPR gene-editing system for the treatment of low back pain comprises one or more sgRNAs targeting exon 11 of hTNFRSF3. In some embodiments, the CRISPR gene-editing system for the treatment of low back pain comprises one or more sgRNAs targeting exon 12 of hTNFRSF3. In some embodiments, the CRISPR gene-editing system for the treatment of low back pain comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTNFRSF3. In some embodiments, the CRISPR gene-editing system for the treatment of low back pain comprises one or more sgRNAs targeting exon 2 of cTNFRSF3. In some embodiments, the CRISPR gene-editing system for the treatment of low back pain comprises one or more sgRNAs targeting exon 3 of cTNFRSF3. In some embodiments, the CRISPR gene-editing system for the treatment of low back pain comprises one or more sgRNAs targeting exon 4 of cTNFRSF3. In some embodiments, the CRISPR gene-editing system for the treatment of low back pain comprises one or more sgRNAs targeting exon 5 of cTNFRSF3. In some embodiments, the CRISPR gene-editing system for the treatment of low back pain comprises one or more sgRNAs targeting exon 6 of cTNFRSF3. In some embodiments, the CRISPR gene-editing system for the treatment of low back pain comprises one or more sgRNAs targeting exon 7 of cTNFRSF3. In some embodiments, the CRISPR gene-editing system for the treatment of low back pain comprises one or more sgRNAs targeting exon 8 of cTNFRSF3. In some embodiments, the CRISPR gene-editing system for the treatment of low back pain comprises one or more sgRNAs targeting exon 9 of cTNFRSF3. In some embodiments, the CRISPR gene-editing system for the treatment of low back pain comprises one or more sgRNAs targeting exon 10 of cTNFRSF3. In some embodiments, the CRISPR gene-editing system for the treatment of low back pain comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTNFRSF3. In some embodiments, the CRISPR gene-editing system for the treatment of low back pain comprises one or more sgRNAs targeting exon 2 of eTNFRSF3. In some embodiments, the CRISPR gene-editing system for the treatment of low back pain comprises one or more sgRNAs targeting exon 3 of eTNFRSF3. In some embodiments, the CRISPR gene-editing system for the treatment of low back pain comprises one or more sgRNAs targeting exon 4 of eTNFRSF3. In some embodiments, the CRISPR gene-editing system for the treatment of low back pain comprises one or more sgRNAs targeting exon 5 of eTNFRSF3. In some embodiments, the CRISPR gene-editing system for the treatment of low back pain comprises one or more sgRNAs targeting exon 6 of eTNFRSF3. In some embodiments, the CRISPR gene-editing system for the treatment of low back pain comprises one or more sgRNAs targeting exon 7 of eTNFRSF3. In some embodiments, the CRISPR gene-editing system for the treatment of low back pain comprises one or more sgRNAs targeting exon 8 of eTNFRSF3. In some embodiments, the CRISPR gene-editing system for the treatment of low back pain comprises one or more sgRNAs targeting exon 9 of eTNFRSF3. In some embodiments, the CRISPR gene-editing system for the treatment of low back pain comprises one or more sgRNAs targeting exon 10 of eTNFRSF3. In some embodiments, the CRISPR gene-editing system for the treatment of low back pain comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTNFRSF3. In some embodiments, the CRISPR gene-editing system for the treatment of low back pain comprises one or more sgRNAs targeting exon 2 of fTNFRSF3. In some embodiments, the CRISPR gene-editing system for the treatment of low back pain comprises one or more sgRNAs targeting exon 3 of fTNFRSF3. In some embodiments, the CRISPR gene-editing system for the treatment of low back pain comprises one or more sgRNAs targeting exon 4 of fTNFRSF3. In some embodiments, the CRISPR gene-editing system for the treatment of low back pain comprises one or more sgRNAs targeting exon 5 of fTNFRSF3. In some embodiments, the CRISPR gene-editing system for the treatment of low back pain comprises one or more sgRNAs targeting exon 6 of fTNFRSF3. In some embodiments, the CRISPR gene-editing system for the treatment of low back pain comprises one or more sgRNAs targeting exon 7 of fTNFRSF3. In some embodiments, the CRISPR gene-editing system for the treatment of low back pain comprises one or more sgRNAs targeting exon 8 of fTNFRSF3. In some embodiments, the CRISPR gene-editing system for the treatment of low back pain comprises one or more sgRNAs targeting exon 9 of fTNFRSF3. In some embodiments, the CRISPR gene-editing system for the treatment of low back pain comprises one or more sgRNAs targeting exon 10 of fTNFRSF3. In some embodiments, the CRISPR gene-editing system for the treatment of low back pain comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TNFRSF4. In some embodiments, the CRISPR gene-editing system for the treatment of low back pain comprises one or more sgRNAs targeting hTNFRSF4. In some embodiments, the CRISPR gene-editing system for the treatment of low back pain comprises one or more sgRNAs targeting cTNFRSF4. In some embodiments, the CRISPR gene-editing system for the treatment of low back pain comprises one or more sgRNAs targeting eTNFRSF4. In some embodiments, the CRISPR gene-editing system for the treatment of low back pain comprises one or more sgRNAs targeting fTNFRSF4. In some embodiments, the CRISPR gene-editing system for the treatment of low back pain comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTNFRSF4. In some embodiments, the CRISPR gene-editing system for the treatment of low back pain comprises one or more sgRNAs targeting exon 2 of hTNFRSF4. In some embodiments, the CRISPR gene-editing system for the treatment of low back pain comprises one or more sgRNAs targeting exon 3 of hTNFRSF4. In some embodiments, the CRISPR gene-editing system for the treatment of low back pain comprises one or more sgRNAs targeting exon 4 of hTNFRSF4. In some embodiments, the CRISPR gene-editing system for the treatment of low back pain comprises one or more sgRNAs targeting exon 5 of hTNFRSF4. In some embodiments, the CRISPR gene-editing system for the treatment of low back pain comprises one or more sgRNAs targeting exon 6 of hTNFRSF4. In some embodiments, the CRISPR gene-editing system for the treatment of low back pain comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTNFRSF4. In some embodiments, the CRISPR gene-editing system for the treatment of low back pain comprises one or more sgRNAs targeting exon 2 of cTNFRSF4. In some embodiments, the CRISPR gene-editing system for the treatment of low back pain comprises one or more sgRNAs targeting exon 3 of cTNFRSF4. In some embodiments, the CRISPR gene-editing system for the treatment of low back pain comprises one or more sgRNAs targeting exon 4 of cTNFRSF4. In some embodiments, the CRISPR gene-editing system for the treatment of low back pain comprises one or more sgRNAs targeting exon 5 of cTNFRSF4. In some embodiments, the CRISPR gene-editing system for the treatment of low back pain comprises one or more sgRNAs targeting exon 6 of cTNFRSF4. In some embodiments, the CRISPR gene-editing system for the treatment of low back pain comprises one or more sgRNAs targeting exon 7 of cTNFRSF4. In some embodiments, the CRISPR gene-editing system for the treatment of low back pain comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTNFRSF4. In some embodiments, the CRISPR gene-editing system for the treatment of low back pain comprises one or more sgRNAs targeting exon 2 of eTNFRSF4. In some embodiments, the CRISPR gene-editing system for the treatment of low back pain comprises one or more sgRNAs targeting exon 3 of eTNFRSF4. In some embodiments, the CRISPR gene-editing system for the treatment of low back pain comprises one or more sgRNAs targeting exon 4 of eTNFRSF4. In some embodiments, the CRISPR gene-editing system for the treatment of low back pain comprises one or more sgRNAs targeting exon 5 of eTNFRSF4. In some embodiments, the CRISPR gene-editing system for the treatment of low back pain comprises one or more sgRNAs targeting exon 6 of eTNFRSF4. In some embodiments, the CRISPR gene-editing system for the treatment of low back pain comprises one or more sgRNAs targeting exon 7 of eTNFRSF4. In some embodiments, the CRISPR gene-editing system for the treatment of low back pain comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTNFRSF4. In some embodiments, the CRISPR gene-editing system for the treatment of low back pain comprises one or more sgRNAs targeting exon 2 of fTNFRSF4. In some embodiments, the CRISPR gene-editing system for the treatment of low back pain comprises one or more sgRNAs targeting exon 3 of fTNFRSF4. In some embodiments, the CRISPR gene-editing system for the treatment of low back pain comprises one or more sgRNAs targeting exon 4 of fTNFRSF4. In some embodiments, the CRISPR gene-editing system for the treatment of low back pain comprises one or more sgRNAs targeting exon 5 of fTNFRSF4. In some embodiments, the CRISPR gene-editing system for the treatment of low back pain comprises one or more sgRNAs targeting exon 6 of fTNFRSF4. In some embodiments, the CRISPR gene-editing system for the treatment of low back pain comprises one or more sgRNAs targeting exon 7 of fTNFRSF4. In some embodiments, the CRISPR gene-editing system for the treatment of low back pain comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting TNFRSF11A. In some embodiments, the CRISPR gene-editing system for the treatment of low back pain comprises one or more sgRNAs targeting hTNFRSF11A. In some embodiments, the CRISPR gene-editing system for the treatment of low back pain comprises one or more sgRNAs targeting cTNFRSF11A. In some embodiments, the CRISPR gene-editing system for the treatment of low back pain comprises one or more sgRNAs targeting eTNFRSF11A. In some embodiments, the CRISPR gene-editing system for the treatment of low back pain comprises one or more sgRNAs targeting fTNFRSF11A. In some embodiments, the CRISPR gene-editing system for the treatment of low back pain comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of hTNFRSF11A. In some embodiments, the CRISPR gene-editing system for the treatment of low back pain comprises one or more sgRNAs targeting exon 2 of hTNFRSF11A. In some embodiments, the CRISPR gene-editing system for the treatment of low back pain comprises one or more sgRNAs targeting exon 3 of hTNFRSF11A. In some embodiments, the CRISPR gene-editing system for the treatment of low back pain comprises one or more sgRNAs targeting exon 4 of hTNFRSF11A. In some embodiments, the CRISPR gene-editing system for the treatment of low back pain comprises one or more sgRNAs targeting exon 5 of hTNFRSF11A. In some embodiments, the CRISPR gene-editing system for the treatment of low back pain comprises one or more sgRNAs targeting exon 6 of hTNFRSF11A. In some embodiments, the CRISPR gene-editing system for the treatment of low back pain comprises one or more sgRNAs targeting exon 7 of hTNFRSF11A. In some embodiments, the CRISPR gene-editing system for the treatment of low back pain comprises one or more sgRNAs targeting exon 8 of hTNFRSF11A. In some embodiments, the CRISPR gene-editing system for the treatment of low back pain comprises one or more sgRNAs targeting exon 9 of hTNFRSF11A. In some embodiments, the CRISPR gene-editing system for the treatment of low back pain comprises one or more sgRNAs targeting exon 10 of hTNFRSF11A. In some embodiments, the CRISPR gene-editing system for the treatment of low back pain comprises one or more sgRNAs targeting exon 11 of hTNFRSF11A. In some embodiments, the CRISPR gene-editing system for the treatment of low back pain comprises one or more sgRNAs targeting exon 12 of hTNFRSF11A. In some embodiments, the CRISPR gene-editing system for the treatment of low back pain comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of cTNFRSF11A. In some embodiments, the CRISPR gene-editing system for the treatment of low back pain comprises one or more sgRNAs targeting exon 2 of cTNFRSF11A. In some embodiments, the CRISPR gene-editing system for the treatment of low back pain comprises one or more sgRNAs targeting exon 3 of cTNFRSF11A. In some embodiments, the CRISPR gene-editing system for the treatment of low back pain comprises one or more sgRNAs targeting exon 4 of cTNFRSF11A. In some embodiments, the CRISPR gene-editing system for the treatment of low back pain comprises one or more sgRNAs targeting exon 5 of cTNFRSF11A. In some embodiments, the CRISPR gene-editing system for the treatment of low back pain comprises one or more sgRNAs targeting exon 6 of cTNFRSF11A. In some embodiments, the CRISPR gene-editing system for the treatment of low back pain comprises one or more sgRNAs targeting exon 7 of cTNFRSF11A. In some embodiments, the CRISPR gene-editing system for the treatment of low back pain comprises one or more sgRNAs targeting exon 8 of cTNFRSF11A. In some embodiments, the CRISPR gene-editing system for the treatment of low back pain comprises one or more sgRNAs targeting exon 9 of cTNFRSF11A. In some embodiments, the CRISPR gene-editing system for the treatment of low back pain comprises one or more sgRNAs targeting exon 10 of cTNFRSF11A. In some embodiments, the CRISPR gene-editing system for the treatment of low back pain comprises one or more sgRNAs targeting exon 11 of cTNFRSF11A. In some embodiments, the CRISPR gene-editing system for the treatment of low back pain comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of eTNFRSF11A. In some embodiments, the CRISPR gene-editing system for the treatment of low back pain comprises one or more sgRNAs targeting exon 2 of eTNFRSF11A. In some embodiments, the CRISPR gene-editing system for the treatment of low back pain comprises one or more sgRNAs targeting exon 3 of eTNFRSF11A. In some embodiments, the CRISPR gene-editing system for the treatment of low back pain comprises one or more sgRNAs targeting exon 4 of eTNFRSF11A. In some embodiments, the CRISPR gene-editing system for the treatment of low back pain comprises one or more sgRNAs targeting exon 5 of eTNFRSF11A. In some embodiments, the CRISPR gene-editing system for the treatment of low back pain comprises one or more sgRNAs targeting exon 6 of eTNFRSF11A. In some embodiments, the CRISPR gene-editing system for the treatment of low back pain comprises one or more sgRNAs targeting exon 7 of eTNFRSF11A. In some embodiments, the CRISPR gene-editing system for the treatment of low back pain comprises one or more sgRNAs targeting exon 8 of eTNFRSF11A. In some embodiments, the CRISPR gene-editing system for the treatment of low back pain comprises one or more sgRNAs targeting exon 9 of eTNFRSF11A. In some embodiments, the CRISPR gene-editing system for the treatment of low back pain comprises one or more sgRNAs targeting exon 10 of eTNFRSF11A. In some embodiments, the CRISPR gene-editing system for the treatment of low back pain comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of low back pain comprises one or more sgRNAs targeting exon 1 of fTNFRSF11A. In some embodiments, the CRISPR gene-editing system for the treatment of low back pain comprises one or more sgRNAs targeting exon 2 of fTNFRSF11A. In some embodiments, the CRISPR gene-editing system for the treatment of low back pain comprises one or more sgRNAs targeting exon 3 of fTNFRSF11A. In some embodiments, the CRISPR gene-editing system for the treatment of low back pain comprises one or more sgRNAs targeting exon 4 of fTNFRSF11A. In some embodiments, the CRISPR gene-editing system for the treatment of low back pain comprises one or more sgRNAs targeting exon 5 of fTNFRSF11A. In some embodiments, the CRISPR gene-editing system for the treatment of low back pain comprises one or more sgRNAs targeting exon 6 of fTNFRSF11A. In some embodiments, the CRISPR gene-editing system for the treatment of low back pain comprises one or more sgRNAs targeting exon 7 of fTNFRSF11A. In some embodiments, the CRISPR gene-editing system for the treatment of low back pain comprises one or more sgRNAs targeting exon 8 of fTNFRSF11A. In some embodiments, the CRISPR gene-editing system for the treatment of low back pain comprises one or more sgRNAs targeting exon 9 of fTNFRSF11A. In some embodiments, the CRISPR gene-editing system for the treatment of low back pain comprises one or more sgRNAs targeting exon 10 of fTNFRSF11A. In some embodiments, the CRISPR gene-editing system for the treatment of low back pain comprises one or more sgRNAs targeting exon 11 of fTNFRSF11A. In some embodiments, the CRISPR gene-editing system for the treatment of low back pain comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### C. DDD

In one aspect, the present disclosure encompasses treatments for degenerative disc disorder (DDD). In some embodiments, the DDD treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the DDD treatment comprises a CRISPR gene-editing system targeting hIL1R1. In some embodiments, the DDD treatment comprises a CRISPR gene-editing system targeting cIL1R1. In some embodiments, the DDD treatment comprises a CRISP gene-editing system R targeting eIL1R1. In some embodiments, the DDD treatment comprises a CRISPR gene-editing system targeting fIL1R1. In some embodiments, the CRISPR gene-editing system for the treatment of DDD comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hIL1R1. In some embodiments, the CRISPR gene-editing system for the treatment of DDD comprises one or more sgRNAs targeting exon 2 of hIL1R1. In some embodiments, the CRISPR gene-editing system for the treatment of DDD comprises one or more sgRNAs targeting exon 3 of hIL1R1. In some embodiments, the CRISPR gene-editing system for the treatment of DDD comprises one or more sgRNAs targeting exon 4 of hIL1R1. In some embodiments, the CRISPR gene-editing system for the treatment of DDD comprises one or more sgRNAs targeting exon 5 of hIL1R1. In some embodiments, the CRISPR gene-editing system for the treatment of DDD comprises one or more sgRNAs targeting exon 6 of hIL1R1. In some embodiments, the CRISPR gene-editing system for the treatment of DDD comprises one or more sgRNAs targeting exon 7 of hIL1R1. In some embodiments, the CRISPR gene-editing system for the treatment of DDD comprises one or more sgRNAs targeting exon 8 of hIL1R1. In some embodiments, the CRISPR gene-editing system for the treatment of DDD comprises one or more sgRNAs targeting exon 9 of hIL1R1. In some embodiments, the CRISPR gene-editing system for the treatment of DDD comprises one or more sgRNAs targeting exon 10 of hIL1R1. In some embodiments, the CRISPR gene-editing system for the treatment of DDD comprises one or more sgRNAs targeting exon 11 of hIL1R1. In some embodiments, the CRISPR gene-editing system for the treatment of DDD comprises one or more sgRNAs targeting exon 12 of hIL1R1. In some embodiments, the CRISPR gene-editing system for the treatment of DDD comprises one or more sgRNAs targeting exon 13 of hIL1R1. In some embodiments, the CRISPR gene-editing system for the treatment of DDD comprises one or more sgRNAs targeting exon 14 of hIL1R1. In some embodiments, the CRISPR gene-editing system for the treatment of DDD comprises one or more sgRNAs targeting exon 15 of hIL1R1. In some embodiments, the CRISPR gene-editing system for the treatment of DDD comprises one or more sgRNAs targeting exon 16 of hIL1R1. In some embodiments, the CRISPR gene-editing system for the treatment of DDD comprises one or more sgRNAs targeting exon 17 of hIL1R1. In some embodiments, the CRISPR gene-editing system for the treatment of DDD comprises one or more sgRNAs targeting exon 18 of hIL1R1. In some embodiments, the CRISPR gene-editing system for the treatment of DDD comprises one or more sgRNAs targeting exon 19 of hIL1R1. In some embodiments, the CRISPR gene-editing system for the treatment of DDD comprises one or more sgRNAs targeting exon 20 of hIL1R1. In some embodiments, the CRISPR gene-editing system for the treatment of DDD comprises one or more sgRNAs targeting exon 21 of hIL1R1. In some embodiments, the CRISPR gene-editing system for the treatment of DDD comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cIL1R1. In some embodiments, the CRISPR gene-editing system for the treatment of DDD comprises one or more sgRNAs targeting exon 2 of cIL1R1. In some embodiments, the CRISPR gene-editing system for the treatment of DDD comprises one or more sgRNAs targeting exon 3 of cIL1R1. In some embodiments, the CRISPR gene-editing system for the treatment of DDD comprises one or more sgRNAs targeting exon 4 of cIL1R1. In some embodiments, the CRISPR gene-editing system for the treatment of DDD comprises one or more sgRNAs targeting exon 5 of cIL1R1. In some embodiments, the CRISPR gene-editing system for the treatment of DDD comprises one or more sgRNAs targeting exon 6 of cIL1R1. In some embodiments, the CRISPR gene-editing system for the treatment of DDD comprises one or more sgRNAs targeting exon 7 of cIL1R1. In some embodiments, the CRISPR gene-editing system for the treatment of DDD comprises one or more sgRNAs targeting exon 8 of cIL1R1. In some embodiments, the CRISPR gene-editing system for the treatment of DDD comprises one or more sgRNAs targeting exon 9 of cIL1R1. In some embodiments, the CRISPR gene-editing system for the treatment of DDD comprises one or more sgRNAs targeting exon 10 of cIL1R1. In some embodiments, the CRISPR gene-editing system for the treatment of DDD comprises one or more sgRNAs targeting exon 11 of cIL1R1. In some embodiments, the CRISPR gene-editing system for the treatment of DDD comprises one or more sgRNAs targeting exon 12 of cIL1R1. In some embodiments, the CRISPR gene-editing system for the treatment of DDD comprises one or more sgRNAs targeting exon 13 of cIL1R1. In some embodiments, the CRISPR gene-editing system for the treatment of DDD comprises one or more sgRNAs targeting exon 14 of cIL1R1. In some embodiments, the CRISPR gene-editing system for the treatment of DDD comprises one or more sgRNAs targeting exon 15 of cIL1R1. In some embodiments, the CRISPR gene-editing system for the treatment of DDD comprises one or more sgRNAs targeting exon 16 of cIL1R1. In some embodiments, the CRISPR gene-editing system for the treatment of DDD comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eIL1R1. In some embodiments, the CRISPR gene-editing system for the treatment of DDD comprises one or more sgRNAs targeting exon 2 of eIL1R1. In some embodiments, the CRISPR gene-editing system for the treatment of DDD comprises one or more sgRNAs targeting exon 3 of eIL1R1. In some embodiments, the CRISPR gene-editing system for the treatment of DDD comprises one or more sgRNAs targeting exon 4 of eIL1R1. In some embodiments, the CRISPR gene-editing system for the treatment of DDD comprises one or more sgRNAs targeting exon 5 of eIL1R1. In some embodiments, the CRISPR gene-editing system for the treatment of DDD comprises one or more sgRNAs targeting exon 6 of eIL1R1. In some embodiments, the CRISPR gene-editing system for the treatment of DDD comprises one or more sgRNAs targeting exon 7 of eIL1R1. In some embodiments, the CRISPR gene-editing system for the treatment of DDD comprises one or more sgRNAs targeting exon 8 of eIL1R1. In some embodiments, the CRISPR gene-editing system for the treatment of DDD comprises one or more sgRNAs targeting exon 9 of eIL1R1. In some embodiments, the CRISPR gene-editing system for the treatment of DDD comprises one or more sgRNAs targeting exon 10 of eIL1R1. In some embodiments, the CRISPR gene-editing system for the treatment of DDD comprises one or more sgRNAs targeting exon 11 of eIL1R1. In some embodiments, the CRISPR gene-editing system for the treatment of DDD comprises one or more sgRNAs targeting exon 12 of eIL1R1. In some embodiments, the CRISPR gene-editing system for the treatment of DDD comprises one or more sgRNAs targeting exon 13 of eIL1R1. In some embodiments, the CRISPR gene-editing system for the treatment of DDD comprises one or more sgRNAs targeting exon 14 of eIL1R1. In some embodiments, the CRISPR gene-editing system for the treatment of DDD comprises one or more sgRNAs targeting exon 15 of eIL1R1. In some embodiments, the CRISPR gene-editing system for the treatment of DDD comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fIL1R1. In some embodiments, the CRISPR gene-editing system for the treatment of DDD comprises one or more sgRNAs targeting exon 2 of fIL1R1. In some embodiments, the CRISPR gene-editing system for the treatment of DDD comprises one or more sgRNAs targeting exon 3 of fIL1R1. In some embodiments, the CRISPR gene-editing system for the treatment of DDD comprises one or more sgRNAs targeting exon 4 of fIL1R1. In some embodiments, the CRISPR gene-editing system for the treatment of DDD comprises one or more sgRNAs targeting exon 5 of fIL1R1. In some embodiments, the CRISPR gene-editing system for the treatment of DDD comprises one or more sgRNAs targeting exon 6 of fIL1R1. In some embodiments, the CRISPR gene-editing system for the treatment of DDD comprises one or more sgRNAs targeting exon 7 of fIL1R1. In some embodiments, the CRISPR gene-editing system for the treatment of DDD comprises one or more sgRNAs targeting exon 8 of fIL1R1. In some embodiments, the CRISPR gene-editing system for the treatment of DDD comprises one or more sgRNAs targeting exon 9 of fIL1R1. In some embodiments, the CRISPR gene-editing system for the treatment of DDD comprises one or more sgRNAs targeting exon 10 of fIL1R1. In some embodiments, the CRISPR gene-editing system for the treatment of DDD comprises one or more sgRNAs targeting exon 11 of fIL1R1. In some embodiments, the CRISPR gene-editing system for the treatment of DDD comprises one or more sgRNAs targeting exon 12 of fIL1R1. In some embodiments, the CRISPR gene-editing system for the treatment of DDD comprises one or more sgRNAs targeting exon 13 of fIL1R1. In some embodiments, the CRISPR gene-editing system for the treatment of DDD comprises one or more sgRNAs targeting exon 14 of fIL1R1. In some embodiments, the CRISPR gene-editing system for the treatment of DDD comprises one or more sgRNAs targeting exon 15 of fIL1R1. In some embodiments, the CRISPR gene-editing system for the treatment of DDD comprises one or more sgRNAs targeting exon 16 of fIL1R1. In some embodiments, the CRISPR gene-editing system for the treatment of DDD comprises one or more sgRNAs targeting exon 17 of fIL1R1. In some embodiments, the CRISPR gene-editing system for the treatment of DDD comprises one or more sgRNAs targeting exon 18 of fIL1R1. In some embodiments, the CRISPR gene-editing system for the treatment of DDD comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting IL1RAP. In some embodiments, the CRISPR gene-editing system for the treatment of DDD comprises one or more sgRNAs targeting hIL1RAP. In some embodiments, the CRISPR gene-editing system for the treatment of DDD comprises one or more sgRNAs targeting cIL1RAP. In some embodiments, the CRISPR gene-editing system for the treatment of DDD comprises one or more sgRNAs targeting eIL1RAP. In some embodiments, the CRISPR gene-editing system for the treatment of DDD comprises one or more sgRNAs targeting fIL1RAP. In some embodiments, the CRISPR gene-editing system for the treatment of DDD comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hIL1RAP. In some embodiments, the CRISPR gene-editing system for the treatment of DDD comprises one or more sgRNAs targeting exon 2 of hIL1RAP. In some embodiments, the CRISPR gene-editing system for the treatment of DDD comprises one or more sgRNAs targeting exon 3 of hIL1RAP. In some embodiments, the CRISPR gene-editing system for the treatment of DDD comprises one or more sgRNAs targeting exon 4 of hIL1RAP. In some embodiments, the CRISPR gene-editing system for the treatment of DDD comprises one or more sgRNAs targeting exon 5 of hIL1RAP. In some embodiments, the CRISPR gene-editing system for the treatment of DDD comprises one or more sgRNAs targeting exon 6 of hIL1RAP. In some embodiments, the CRISPR gene-editing system for the treatment of DDD comprises one or more sgRNAs targeting exon 7 of hIL1RAP. In some embodiments, the CRISPR gene-editing system for the treatment of DDD comprises one or more sgRNAs targeting exon 8 of hIL1RAP. In some embodiments, the CRISPR gene-editing system for the treatment of DDD comprises one or more sgRNAs targeting exon 9 of hIL1RAP. In some embodiments, the CRISPR gene-editing system for the treatment of DDD comprises one or more sgRNAs targeting exon 10 of hIL1RAP. In some embodiments, the CRISPR gene-editing system for the treatment of DDD comprises one or more sgRNAs targeting exon 11 of hIL1RAP. In some embodiments, the CRISPR gene-editing system for the treatment of DDD comprises one or more sgRNAs targeting exon 12 of hIL1RAP. In some embodiments, the CRISPR gene-editing system for the treatment of DDD comprises one or more sgRNAs targeting exon 13 of hIL1RAP. In some embodiments, the CRISPR gene-editing system for the treatment of DDD comprises one or more sgRNAs targeting exon 14 of hIL1RAP. In some embodiments, the CRISPR gene-editing system for the treatment of DDD comprises one or more sgRNAs targeting exon 15 of hIL1RAP. In some embodiments, the CRISPR gene-editing system for the treatment of DDD comprises one or more sgRNAs targeting exon 16 of hIL1RAP. In some embodiments, the CRISPR gene-editing system for the treatment of DDD comprises one or more sgRNAs targeting exon 17 of hIL1RAP. In some embodiments, the CRISPR gene-editing system for the treatment of DDD comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cIL1RAP. In some embodiments, the CRISPR gene-editing system for the treatment of DDD comprises one or more sgRNAs targeting exon 2 of cIL1RAP. In some embodiments, the CRISPR gene-editing system for the treatment of DDD comprises one or more sgRNAs targeting exon 3 of cIL1RAP. In some embodiments, the CRISPR gene-editing system for the treatment of DDD comprises one or more sgRNAs targeting exon 4 of cIL1RAP. In some embodiments, the CRISPR gene-editing system for the treatment of DDD comprises one or more sgRNAs targeting exon 5 of cIL1RAP. In some embodiments, the CRISPR gene-editing system for the treatment of DDD comprises one or more sgRNAs targeting exon 6 of cIL1RAP. In some embodiments, the CRISPR gene-editing system for the treatment of DDD comprises one or more sgRNAs targeting exon 7 of cIL1RAP. In some embodiments, the CRISPR gene-editing system for the treatment of DDD comprises one or more sgRNAs targeting exon 8 of cIL1RAP. In some embodiments, the CRISPR gene-editing system for the treatment of DDD comprises one or more sgRNAs targeting exon 9 of cIL1RAP. In some embodiments, the CRISPR gene-editing system for the treatment of DDD comprises one or more sgRNAs targeting exon 10 of cIL1RAP. In some embodiments, the CRISPR gene-editing system for the treatment of DDD comprises one or more sgRNAs targeting exon 11 of cIL1RAP. In some embodiments, the CRISPR gene-editing system for the treatment of DDD comprises one or more sgRNAs targeting exon 12 of cIL1RAP. In some embodiments, the CRISPR gene-editing system for the treatment of DDD comprises one or more sgRNAs targeting exon 13 of cIL1RAP. In some embodiments, the CRISPR gene-editing system for the treatment of DDD comprises one or more sgRNAs targeting exon 14 of cIL1RAP. In some embodiments, the CRISPR gene-editing system for the treatment of DDD comprises one or more sgRNAs targeting exon 15 of cIL1RAP. In some embodiments, the CRISPR gene-editing system for the treatment of DDD comprises one or more sgRNAs targeting exon 16 of cIL1RAP. In some embodiments, the CRISPR gene-editing system for the treatment of DDD comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eIL1RAP. In some embodiments, the CRISPR gene-editing system for the treatment of DDD comprises one or more sgRNAs targeting exon 2 of eIL1RAP. In some embodiments, the CRISPR gene-editing system for the treatment of DDD comprises one or more sgRNAs targeting exon 3 of eIL1RAP. In some embodiments, the CRISPR gene-editing system for the treatment of DDD comprises one or more sgRNAs targeting exon 4 of eIL1RAP. In some embodiments, the CRISPR gene-editing system for the treatment of DDD comprises one or more sgRNAs targeting exon 5 of eIL1RAP. In some embodiments, the CRISPR gene-editing system for the treatment of DDD comprises one or more sgRNAs targeting exon 6 of eIL1RAP. In some embodiments, the CRISPR gene-editing system for the treatment of DDD comprises one or more sgRNAs targeting exon 7 of eIL1RAP. In some embodiments, the CRISPR gene-editing system for the treatment of DDD comprises one or more sgRNAs targeting exon 8 of eIL1RAP. In some embodiments, the CRISPR gene-editing system for the treatment of DDD comprises one or more sgRNAs targeting exon 9 of eIL1RAP. In some embodiments, the CRISPR gene-editing system for the treatment of DDD comprises one or more sgRNAs targeting exon 10 of eIL1RAP. In some embodiments, the CRISPR gene-editing system for the treatment of DDD comprises one or more sgRNAs targeting exon 11 of eIL1RAP. In some embodiments, the CRISPR gene-editing system for the treatment of DDD comprises one or more sgRNAs targeting exon 12 of eIL1RAP. In some embodiments, the CRISPR gene-editing system for the treatment of DDD comprises one or more sgRNAs targeting exon 13 of eIL1RAP. In some embodiments, the CRISPR gene-editing system for the treatment of DDD comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fIL1RAP. In some embodiments, the CRISPR gene-editing system for the treatment of DDD comprises one or more sgRNAs targeting exon 2 of fIL1RAP. In some embodiments, the CRISPR gene-editing system for the treatment of DDD comprises one or more sgRNAs targeting exon 3 of fIL1RAP. In some embodiments, the CRISPR gene-editing system for the treatment of DDD comprises one or more sgRNAs targeting exon 4 of fIL1RAP. In some embodiments, the CRISPR gene-editing system for the treatment of DDD comprises one or more sgRNAs targeting exon 5 of fIL1RAP. In some embodiments, the CRISPR gene-editing system for the treatment of DDD comprises one or more sgRNAs targeting exon 6 of fIL1RAP. In some embodiments, the CRISPR gene-editing system for the treatment of DDD comprises one or more sgRNAs targeting exon 7 of fIL1RAP. In some embodiments, the CRISPR gene-editing system for the treatment of DDD comprises one or more sgRNAs targeting exon 8 of fIL1RAP. In some embodiments, the CRISPR gene-editing system for the treatment of DDD comprises one or more sgRNAs targeting exon 9 of fIL1RAP. In some embodiments, the CRISPR gene-editing system for the treatment of DDD comprises one or more sgRNAs targeting exon 10 of fIL1RAP. In some embodiments, the CRISPR gene-editing system for the treatment of DDD comprises one or more sgRNAs targeting exon 11 of fIL1RAP. In some embodiments, the CRISPR gene-editing system for the treatment of DDD comprises one or more sgRNAs targeting exon 12 of fIL1RAP. In some embodiments, the CRISPR gene-editing system for the treatment of DDD comprises one or more sgRNAs targeting exon 13 of fIL1RAP. In some embodiments, the CRISPR gene-editing system for the treatment of DDD comprises one or more sgRNAs targeting exon 14 of fIL1RAP. In some embodiments, the CRISPR gene-editing system for the treatment of DDD comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TGFBR1. In some embodiments, the CRISPR gene-editing system for the treatment of DDD comprises one or more sgRNAs targeting hTGFBR1. In some embodiments, the CRISPR gene-editing system for the treatment of DDD comprises one or more sgRNAs targeting cTGFBR1. In some embodiments, the CRISPR gene-editing system for the treatment of DDD comprises one or more sgRNAs targeting e TGFBR1. In some embodiments, the CRISPR gene-editing system for the treatment of DDD comprises one or more sgRNAs targeting fTGFBR1. In some embodiments, the CRISPR gene-editing system for the treatment of DDD comprising one or more sgRNAs targeting an exon of TGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTGFBR1. In some embodiments, the CRISPR gene-editing system for the treatment of DDD comprises one or more sgRNAs targeting exon 2 of hTGFBR1. In some embodiments, the CRISPR gene-editing system for the treatment of DDD comprises one or more sgRNAs targeting exon 3 of hTGFBR1. In some embodiments, the CRISPR gene-editing system for the treatment of DDD comprises one or more sgRNAs targeting exon 4 of hTGFBR1. In some embodiments, the CRISPR gene-editing system for the treatment of DDD comprises one or more sgRNAs targeting exon 5 of hTGFBR1. In some embodiments, the CRISPR gene-editing system for the treatment of DDD comprises one or more sgRNAs targeting exon 6 of hTGFBR1. In some embodiments, the CRISPR gene-editing system for the treatment of DDD comprises one or more sgRNAs targeting exon 7 of hTGFBR1. In some embodiments, the CRISPR gene-editing system for the treatment of DDD comprises one or more sgRNAs targeting exon 8 of hTGFBR1. In some embodiments, the CRISPR gene-editing system for the treatment of DDD comprises one or more sgRNAs targeting exon 9 of hTGFBR1. In some embodiments, the CRISPR gene-editing system for the treatment of DDD comprises one or more sgRNAs targeting exon 10 of hTGFBR1. In some embodiments, the CRISPR gene-editing system for the treatment of DDD comprises one or more sgRNAs targeting exon 11 of hTGFBR1. In some embodiments, the CRISPR gene-editing system for the treatment of DDD comprising one or more sgRNAs targeting an exon of hTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTGFBR1. In some embodiments, the CRISPR gene-editing system for the treatment of DDD comprises one or more sgRNAs targeting exon 2 of cTGFBR1. In some embodiments, the CRISPR gene-editing system for the treatment of DDD comprises one or more sgRNAs targeting exon 3 of cTGFBR1. In some embodiments, the CRISPR gene-editing system for the treatment of DDD comprises one or more sgRNAs targeting exon 4 of cTGFBR1. In some embodiments, the CRISPR gene-editing system for the treatment of DDD comprises one or more sgRNAs targeting exon 5 of cTGFBR1. In some embodiments, the CRISPR gene-editing system for the treatment of DDD comprises one or more sgRNAs targeting exon 6 of cTGFBR1. In some embodiments, the CRISPR gene-editing system for the treatment of DDD comprises one or more sgRNAs targeting exon 7 of cTGFBR1. In some embodiments, the CRISPR gene-editing system for the treatment of DDD comprises one or more sgRNAs targeting exon 8 of cTGFBR1. In some embodiments, the CRISPR gene-editing system for the treatment of DDD comprises one or more sgRNAs targeting exon 9 of cTGFBR1. In some embodiments, the CRISPR gene-editing system for the treatment of DDD comprising one or more sgRNAs targeting an exon of cTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTGFBR1. In some embodiments, the CRISPR gene-editing system for the treatment of DDD comprises one or more sgRNAs targeting exon 2 of eTGFBR1. In some embodiments, the CRISPR gene-editing system for the treatment of DDD comprises one or more sgRNAs targeting exon 3 of eTGFBR1. In some embodiments, the CRISPR gene-editing system for the treatment of DDD comprises one or more sgRNAs targeting exon 4 of eTGFBR1. In some embodiments, the CRISPR gene-editing system for the treatment of DDD comprises one or more sgRNAs targeting exon 5 of eTGFBR1. In some embodiments, the CRISPR gene-editing system for the treatment of DDD comprises one or more sgRNAs targeting exon 6 of eTGFBR1. In some embodiments, the CRISPR gene-editing system for the treatment of DDD comprises one or more sgRNAs targeting exon 7 of eTGFBR1. In some embodiments, the CRISPR gene-editing system for the treatment of DDD comprises one or more sgRNAs targeting exon 8 of eTGFBR1. In some embodiments, the CRISPR gene-editing system for the treatment of DDD comprises one or more sgRNAs targeting exon 9 of eTGFBR1. In some embodiments, the CRISPR gene-editing system for the treatment of DDD comprises one or more sgRNAs targeting exon 10 of eTGFBR1. In some embodiments, the CRISPR gene-editing system for the treatment of DDD comprises one or more sgRNAs targeting exon 11 of eTGFBR1. In some embodiments, the CRISPR gene-editing system for the treatment of DDD comprises one or more sgRNAs targeting exon 12 of eTGFBR1. In some embodiments, the CRISPR gene-editing system for the treatment of DDD comprising one or more sgRNAs targeting an exon of eTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTGFBR1. In some embodiments, the CRISPR gene-editing system for the treatment of DDD comprises one or more sgRNAs targeting exon 2 of fTGFBR1. In some embodiments, the CRISPR gene-editing system for the treatment of DDD comprises one or more sgRNAs targeting exon 3 of fTGFBR1. In some embodiments, the CRISPR gene-editing system for the treatment of DDD comprises one or more sgRNAs targeting exon 4 of fTGFBR1. In some embodiments, the CRISPR gene-editing system for the treatment of DDD comprises one or more sgRNAs targeting exon 5 of fTGFBR1. In some embodiments, the CRISPR gene-editing system for the treatment of DDD comprises one or more sgRNAs targeting exon 6 of fTGFBR1. In some embodiments, the CRISPR gene-editing system for the treatment of DDD comprises one or more sgRNAs targeting exon 7 of fTGFBR1. In some embodiments, the CRISPR gene-editing system for the treatment of DDD comprises one or more sgRNAs targeting exon 8 of fTGFBR1. In some embodiments, the CRISPR gene-editing system for the treatment of DDD comprises one or more sgRNAs targeting exon 9 of fTGFBR1. In some embodiments, the CRISPR gene-editing system for the treatment of DDD comprises one or more sgRNAs targeting exon 10 of fTGFBR1. In some embodiments, the CRISPR gene-editing system for the treatment of DDD comprises one or more sgRNAs targeting exon 11 of fTGFBR1. In some embodiments, the CRISPR gene-editing system for the treatment of DDD comprising one or more sgRNAs targeting an exon of fTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TGFBR2. In some embodiments, the CRISPR gene-editing system for the treatment of DDD comprises one or more sgRNAs targeting hTGFBR2. In some embodiments, the CRISPR gene-editing system for the treatment of DDD comprises one or more sgRNAs targeting cTGFBR2. In some embodiments, the CRISPR gene-editing system for the treatment of DDD comprises one or more sgRNAs targeting eTGFBR2. In some embodiments, the CRISPR gene-editing system for the treatment of DDD comprises one or more sgRNAs targeting fTGFBR2. In some embodiments, the CRISPR gene-editing system for the treatment of DDD comprising one or more sgRNAs targeting an exon of TGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTGFBR2. In some embodiments, the CRISPR gene-editing system for the treatment of DDD comprises one or more sgRNAs targeting exon 2 of hTGFBR2. In some embodiments, the CRISPR gene-editing system for the treatment of DDD comprises one or more sgRNAs targeting exon 3 of hTGFBR2. In some embodiments, the CRISPR gene-editing system for the treatment of DDD comprises one or more sgRNAs targeting exon 4 of hTGFBR2. In some embodiments, the CRISPR gene-editing system for the treatment of DDD comprises one or more sgRNAs targeting exon 5 of hTGFBR2. In some embodiments, the CRISPR gene-editing system for the treatment of DDD comprises one or more sgRNAs targeting exon 6 of hTGFBR2. In some embodiments, the CRISPR gene-editing system for the treatment of DDD comprises one or more sgRNAs targeting exon 7 of hTGFBR2. In some embodiments, the CRISPR gene-editing system for the treatment of DDD comprises one or more sgRNAs targeting exon 8 of hTGFBR2. In some embodiments, the CRISPR gene-editing system for the treatment of DDD comprises one or more sgRNAs targeting exon 9 of hTGFBR2. In some embodiments, the CRISPR gene-editing system for the treatment of DDD comprising one or more sgRNAs targeting an exon of hTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTGFBR2. In some embodiments, the CRISPR gene-editing system for the treatment of DDD comprises one or more sgRNAs targeting exon 2 of cTGFBR2. In some embodiments, the CRISPR gene-editing system for the treatment of DDD comprises one or more sgRNAs targeting exon 3 of cTGFBR2. In some embodiments, the CRISPR gene-editing system for the treatment of DDD comprises one or more sgRNAs targeting exon 4 of cTGFBR2. In some embodiments, the CRISPR gene-editing system for the treatment of DDD comprises one or more sgRNAs targeting exon 5 of cTGFBR2. In some embodiments, the CRISPR gene-editing system for the treatment of DDD comprises one or more sgRNAs targeting exon 6 of cTGFBR2. In some embodiments, the CRISPR gene-editing system for the treatment of DDD comprises one or more sgRNAs targeting exon 7 of cTGFBR2. In some embodiments, the CRISPR gene-editing system for the treatment of DDD comprises one or more sgRNAs targeting exon 8 of cTGFBR2. In some embodiments, the CRISPR gene-editing system for the treatment of DDD comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTGFBR2. In some embodiments, the CRISPR gene-editing system for the treatment of DDD comprises one or more sgRNAs targeting exon 2 of eTGFBR2. In some embodiments, the CRISPR gene-editing system for the treatment of DDD comprises one or more sgRNAs targeting exon 3 of eTGFBR2. In some embodiments, the CRISPR gene-editing system for the treatment of DDD comprises one or more sgRNAs targeting exon 4 of eTGFBR2. In some embodiments, the CRISPR gene-editing system for the treatment of DDD comprises one or more sgRNAs targeting exon 5 of eTGFBR2. In some embodiments, the CRISPR gene-editing system for the treatment of DDD comprises one or more sgRNAs targeting exon 6 of eTGFBR2. In some embodiments, the CRISPR gene-editing system for the treatment of DDD comprises one or more sgRNAs targeting exon 7 of eTGFBR2. In some embodiments, the CRISPR gene-editing system for the treatment of DDD comprises one or more sgRNAs targeting exon 8 of eTGFBR2. In some embodiments, the CRISPR gene-editing system for the treatment of DDD comprises one or more sgRNAs targeting exon 9 of eTGFBR2. In some embodiments, the CRISPR gene-editing system for the treatment of DDD comprises one or more sgRNAs targeting exon 10 of eTGFBR2. In some embodiments, the CRISPR gene-editing system for the treatment of DDD comprising one or more sgRNAs targeting an exon of eTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTGFBR2. In some embodiments, the CRISPR gene-editing system for the treatment of DDD comprises one or more sgRNAs targeting exon 2 of fTGFBR2. In some embodiments, the CRISPR gene-editing system for the treatment of DDD comprises one or more sgRNAs targeting exon 3 of fTGFBR2. In some embodiments, the CRISPR gene-editing system for the treatment of DDD comprises one or more sgRNAs targeting exon 4 of fTGFBR2. In some embodiments, the CRISPR gene-editing system for the treatment of DDD comprises one or more sgRNAs targeting exon 5 of fTGFBR2. In some embodiments, the CRISPR gene-editing system for the treatment of DDD comprises one or more sgRNAs targeting exon 6 of fTGFBR2. In some embodiments, the CRISPR gene-editing system for the treatment of DDD comprises one or more sgRNAs targeting exon 7 of fTGFBR2. In some embodiments, the CRISPR gene-editing system for the treatment of DDD comprises one or more sgRNAs targeting exon 8 of fTGFBR2. In some embodiments, the CRISPR gene-editing system for the treatment of DDD comprising one or more sgRNAs targeting an exon of fTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting IL6R. In some embodiments, the CRISPR gene-editing system for the treatment of DDD comprises one or more sgRNAs targeting hIL6R. In some embodiments, the CRISPR gene-editing system for the treatment of DDD comprises one or more sgRNAs targeting cIL6R. In some embodiments, the CRISPR gene-editing system for the treatment of DDD comprises one or more sgRNAs targeting eIL6R. In some embodiments, the CRISPR gene-editing system for the treatment of DDD comprises one or more sgRNAs targeting fIL6R. In some embodiments, the CRISPR gene-editing system for the treatment of DDD comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hIL6R. In some embodiments, the CRISPR gene-editing system for the treatment of DDD comprises one or more sgRNAs targeting exon 2 of hIL6R. In some embodiments, the CRISPR gene-editing system for the treatment of DDD comprises one or more sgRNAs targeting exon 3 of hIL6R. In some embodiments, the CRISPR gene-editing system for the treatment of DDD comprises one or more sgRNAs targeting exon 4 of hIL6R. In some embodiments, the CRISPR gene-editing system for the treatment of DDD comprises one or more sgRNAs targeting exon 5 of hIL6R. In some embodiments, the CRISPR gene-editing system for the treatment of DDD comprises one or more sgRNAs targeting exon 6 of hIL6R. In some embodiments, the CRISPR gene-editing system for the treatment of DDD comprises one or more sgRNAs targeting exon 7 of hIL6R. In some embodiments, the CRISPR gene-editing system for the treatment of DDD comprises one or more sgRNAs targeting exon 8 of hIL6R. In some embodiments, the CRISPR gene-editing system for the treatment of DDD comprises one or more sgRNAs targeting exon 9 of hIL6R. In some embodiments, the CRISPR gene-editing system for the treatment of DDD comprises one or more sgRNAs targeting exon 10 of hIL6R. In some embodiments, the CRISPR gene-editing system for the treatment of DDD comprises one or more sgRNAs targeting exon 11 of hIL6R In some embodiments, the CRISPR gene-editing system for the treatment of DDD comprises one or more sgRNAs targeting exon 12 of hIL6R. In some embodiments, the CRISPR gene-editing system for the treatment of DDD comprises one or more sgRNAs targeting exon 13 of hIL6R. In some embodiments, the CRISPR gene-editing system for the treatment of DDD comprises one or more sgRNAs targeting exon 14 of hIL6R. In some embodiments, the CRISPR gene-editing system for the treatment of DDD comprises one or more sgRNAs targeting exon 15 of hIL6R. In some embodiments, the CRISPR gene-editing system for the treatment of DDD comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cIL6R. In some embodiments, the CRISPR gene-editing system for the treatment of DDD comprises one or more sgRNAs targeting exon 2 of cIL6R. In some embodiments, the CRISPR gene-editing system for the treatment of DDD comprises one or more sgRNAs targeting exon 3 of cIL6R. In some embodiments, the CRISPR gene-editing system for the treatment of DDD comprises one or more sgRNAs targeting exon 4 of cIL6R. In some embodiments, the CRISPR gene-editing system for the treatment of DDD comprises one or more sgRNAs targeting exon 5 of cIL6R. In some embodiments, the CRISPR gene-editing system for the treatment of DDD comprises one or more sgRNAs targeting exon 6 of cIL6R. In some embodiments, the CRISPR gene-editing system for the treatment of DDD comprises one or more sgRNAs targeting exon 7 of cIL6R. In some embodiments, the CRISPR gene-editing system for the treatment of DDD comprises one or more sgRNAs targeting exon 8 of cIL6R. In some embodiments, the CRISPR gene-editing system for the treatment of DDD comprises one or more sgRNAs targeting exon 9 of cIL6R. In some embodiments, the CRISPR gene-editing system for the treatment of DDD comprises one or more sgRNAs targeting exon 10 of cIL6R. In some embodiments, the CRISPR gene-editing system for the treatment of DDD comprises one or more sgRNAs targeting exon 11 of cIL6R. In some embodiments, the CRISPR gene-editing system for the treatment of DDD comprises one or more sgRNAs targeting exon 12 of cIL6R. In some embodiments, the CRISPR gene-editing system for the treatment of DDD comprises one or more sgRNAs targeting exon 13 of cIL6R. In some embodiments, the CRISPR gene-editing system for the treatment of DDD comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eIL6R. In some embodiments, the CRISPR gene-editing system for the treatment of DDD comprises one or more sgRNAs targeting exon 2 of eIL6R. In some embodiments, the CRISPR gene-editing system for the treatment of DDD comprises one or more sgRNAs targeting exon 3 of eIL6R. In some embodiments, the CRISPR gene-editing system for the treatment of DDD comprises one or more sgRNAs targeting exon 4 of eIL6R. In some embodiments, the CRISPR gene-editing system for the treatment of DDD comprises one or more sgRNAs targeting exon 5 of eIL6R. In some embodiments, the CRISPR gene-editing system for the treatment of DDD comprises one or more sgRNAs targeting exon 6 of eIL6R. In some embodiments, the CRISPR gene-editing system for the treatment of DDD comprises one or more sgRNAs targeting exon 7 of eIL6R. In some embodiments, the CRISPR gene-editing system for the treatment of DDD comprises one or more sgRNAs targeting exon 8 of eIL6R. In some embodiments, the CRISPR gene-editing system for the treatment of DDD comprises one or more sgRNAs targeting exon 9 of eIL6R. In some embodiments, the CRISPR gene-editing system for the treatment of DDD comprises one or more sgRNAs targeting exon 10 of eIL6R. In some embodiments, the CRISPR gene-editing system for the treatment of DDD comprises one or more sgRNAs targeting exon 11 of eIL6R. In some embodiments, the CRISPR gene-editing system for the treatment of DDD comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fIL6R. In some embodiments, the CRISPR gene-editing system for the treatment of DDD comprises one or more sgRNAs targeting exon 2 of fIL6R. In some embodiments, the CRISPR gene-editing system for the treatment of DDD comprises one or more sgRNAs targeting exon 3 of fIL6R. In some embodiments, the CRISPR gene-editing system for the treatment of DDD comprises one or more sgRNAs targeting exon 4 of fIL6R. In some embodiments, the CRISPR gene-editing system for the treatment of DDD comprises one or more sgRNAs targeting exon 5 of fIL6R. In some embodiments, the CRISPR gene-editing system for the treatment of DDD comprises one or more sgRNAs targeting exon 6 of fIL6R. In some embodiments, the CRISPR gene-editing system for the treatment of DDD comprises one or more sgRNAs targeting exon 7 of fIL6R. In some embodiments, the CRISPR gene-editing system for the treatment of DDD comprises one or more sgRNAs targeting exon 8 of fIL6R. In some embodiments, the CRISPR gene-editing system for the treatment of DDD comprises one or more sgRNAs targeting exon 9 of fIL6R. In some embodiments, the CRISPR gene-editing system for the treatment of DDD comprises one or more sgRNAs targeting exon 10 of fIL6R. In some embodiments, the CRISPR gene-editing system for the treatment of DDD comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting IL6ST. In some embodiments, the CRISPR gene-editing system for the treatment of DDD comprises one or more sgRNAs targeting hIL6ST. In some embodiments, the CRISPR gene-editing system for the treatment of DDD comprises one or more sgRNAs targeting cIL6ST. In some embodiments, the CRISPR gene-editing system for the treatment of DDD comprises one or more sgRNAs targeting eIL6ST. In some embodiments, the CRISPR gene-editing system for the treatment of DDD comprises one or more sgRNAs targeting fIL6ST. In some embodiments, the CRISPR gene-editing system for the treatment of DDD comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hIL6ST. In some embodiments, the CRISPR gene-editing system for the treatment of DDD comprises one or more sgRNAs targeting exon 2 of hIL6ST. In some embodiments, the CRISPR gene-editing system for the treatment of DDD comprises one or more sgRNAs targeting exon 3 of hIL6ST. In some embodiments, the CRISPR gene-editing system for the treatment of DDD comprises one or more sgRNAs targeting exon 4 of hIL6ST. In some embodiments, the CRISPR gene-editing system for the treatment of DDD comprises one or more sgRNAs targeting exon 5 of hIL6ST. In some embodiments, the CRISPR gene-editing system for the treatment of DDD comprises one or more sgRNAs targeting exon 6 of hIL6ST. In some embodiments, the CRISPR gene-editing system for the treatment of DDD comprises one or more sgRNAs targeting exon 7 of hIL6ST. In some embodiments, the CRISPR gene-editing system for the treatment of DDD comprises one or more sgRNAs targeting exon 8 of hIL6ST. In some embodiments, the CRISPR gene-editing system for the treatment of DDD comprises one or more sgRNAs targeting exon 9 of hIL6ST. In some embodiments, the CRISPR gene-editing system for the treatment of DDD comprises one or more sgRNAs targeting exon 10 of hIL6ST. In some embodiments, the CRISPR gene-editing system for the treatment of DDD comprises one or more sgRNAs targeting exon 11 of hIL6ST. In some embodiments, the CRISPR gene-editing system for the treatment of DDD comprises one or more sgRNAs targeting exon 12 of hIL6ST. In some embodiments, the CRISPR gene-editing system for the treatment of DDD comprises one or more sgRNAs targeting exon 13 of hIL6ST. In some embodiments, the CRISPR gene-editing system for the treatment of DDD comprises one or more sgRNAs targeting exon 14 of hIL6ST. In some embodiments, the CRISPR gene-editing system for the treatment of DDD comprises one or more sgRNAs targeting exon 15 of hIL6ST. In some embodiments, the CRISPR gene-editing system for the treatment of DDD comprises one or more sgRNAs targeting exon 16 of hIL6ST. In some embodiments, the CRISPR gene-editing system for the treatment of DDD comprises one or more sgRNAs targeting exon 17 of hIL6ST. In some embodiments, the CRISPR gene-editing system for the treatment of DDD comprises one or more sgRNAs targeting exon 18 of hIL6ST. In some embodiments, the CRISPR gene-editing system for the treatment of DDD comprises one or more sgRNAs targeting exon 19 of hIL6ST. In some embodiments, the CRISPR gene-editing system for the treatment of DDD comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cIL6ST. In some embodiments, the CRISPR gene-editing system for the treatment of DDD comprises one or more sgRNAs targeting exon 2 of cIL6ST. In some embodiments, the CRISPR gene-editing system for the treatment of DDD comprises one or more sgRNAs targeting exon 3 of cIL6ST. In some embodiments, the CRISPR gene-editing system for the treatment of DDD comprises one or more sgRNAs targeting exon 4 of cIL6ST. In some embodiments, the CRISPR gene-editing system for the treatment of DDD comprises one or more sgRNAs targeting exon 5 of cIL6ST. In some embodiments, the CRISPR gene-editing system for the treatment of DDD comprises one or more sgRNAs targeting exon 6 of cIL6ST. In some embodiments, the CRISPR gene-editing system for the treatment of DDD comprises one or more sgRNAs targeting exon 7 of cIL6ST. In some embodiments, the CRISPR gene-editing system for the treatment of DDD comprises one or more sgRNAs targeting exon 8 of cIL6ST. In some embodiments, the CRISPR gene-editing system for the treatment of DDD comprises one or more sgRNAs targeting exon 9 of cIL6ST. In some embodiments, the CRISPR gene-editing system for the treatment of DDD comprises one or more sgRNAs targeting exon 10 of cIL6ST. In some embodiments, the CRISPR gene-editing system for the treatment of DDD comprises one or more sgRNAs targeting exon 11 of cIL6ST In some embodiments, the CRISPR gene-editing system for the treatment of DDD comprises one or more sgRNAs targeting exon 12 of cIL6ST. In some embodiments, the CRISPR gene-editing system for the treatment of DDD comprises one or more sgRNAs targeting exon 13 of cIL6ST. In some embodiments, the CRISPR gene-editing system for the treatment of DDD comprises one or more sgRNAs targeting exon 14 of cIL6ST. In some embodiments, the CRISPR gene-editing system for the treatment of DDD comprises one or more sgRNAs targeting exon 15 of cIL6ST. In some embodiments, the CRISPR gene-editing system for the treatment of DDD comprises one or more sgRNAs targeting exon 16 of cIL6ST. In some embodiments, the CRISPR gene-editing system for the treatment of DDD comprises one or more sgRNAs targeting exon 17 of cIL6ST. In some embodiments, the CRISPR gene-editing system for the treatment of DDD comprises one or more sgRNAs targeting exon 18 of cIL6ST. In some embodiments, the CRISPR gene-editing system for the treatment of DDD comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eIL6ST. In some embodiments, the CRISPR gene-editing system for the treatment of DDD comprises one or more sgRNAs targeting exon 2 of eIL6ST. In some embodiments, the CRISPR gene-editing system for the treatment of DDD comprises one or more sgRNAs targeting exon 3 of eIL6ST. In some embodiments, the CRISPR gene-editing system for the treatment of DDD comprises one or more sgRNAs targeting exon 4 of eIL6ST. In some embodiments, the CRISPR gene-editing system for the treatment of DDD comprises one or more sgRNAs targeting exon 5 of eIL6ST. In some embodiments, the CRISPR gene-editing system for the treatment of DDD comprises one or more sgRNAs targeting exon 6 of eIL6ST. In some embodiments, the CRISPR gene-editing system for the treatment of DDD comprises one or more sgRNAs targeting exon 7 of eIL6ST. In some embodiments, the CRISPR gene-editing system for the treatment of DDD comprises one or more sgRNAs targeting exon 8 of eIL6ST. In some embodiments, the CRISPR gene-editing system for the treatment of DDD comprises one or more sgRNAs targeting exon 9 of eIL6ST. In some embodiments, the CRISPR gene-editing system for the treatment of DDD comprises one or more sgRNAs targeting exon 10 of eIL6ST. In some embodiments, the CRISPR gene-editing system for the treatment of DDD comprises one or more sgRNAs targeting exon 11 of eIL6ST In some embodiments, the CRISPR gene-editing system for the treatment of DDD comprises one or more sgRNAs targeting exon 12 of eIL6ST. In some embodiments, the CRISPR gene-editing system for the treatment of DDD comprises one or more sgRNAs targeting exon 13 of eIL6ST. In some embodiments, the CRISPR gene-editing system for the treatment of DDD comprises one or more sgRNAs targeting exon 14 of eIL6ST. In some embodiments, the CRISPR gene-editing system for the treatment of DDD comprises one or more sgRNAs targeting exon 15 of eIL6ST. In some embodiments, the CRISPR gene-editing system for the treatment of DDD comprises one or more sgRNAs targeting exon 16 of eIL6ST. In some embodiments, the CRISPR gene-editing system for the treatment of DDD comprises one or more sgRNAs targeting exon 17 of eIL6ST. In some embodiments, the CRISPR gene-editing system for the treatment of DDD comprises one or more sgRNAs targeting exon 18 of eIL6ST. In some embodiments, the CRISPR gene-editing system for the treatment of DDD comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fIL6ST. In some embodiments, the CRISPR gene-editing system for the treatment of DDD comprises one or more sgRNAs targeting exon 2 of fIL6ST. In some embodiments, the CRISPR gene-editing system for the treatment of DDD comprises one or more sgRNAs targeting exon 3 of fIL6ST. In some embodiments, the CRISPR gene-editing system for the treatment of DDD comprises one or more sgRNAs targeting exon 4 of fIL6ST. In some embodiments, the CRISPR gene-editing system for the treatment of DDD comprises one or more sgRNAs targeting exon 5 of fIL6ST. In some embodiments, the CRISPR gene-editing system for the treatment of DDD comprises one or more sgRNAs targeting exon 6 of fIL6ST. In some embodiments, the CRISPR gene-editing system for the treatment of DDD comprises one or more sgRNAs targeting exon 7 of fIL6ST. In some embodiments, the CRISPR gene-editing system for the treatment of DDD comprises one or more sgRNAs targeting exon 8 of fIL6ST. In some embodiments, the CRISPR gene-editing system for the treatment of DDD comprises one or more sgRNAs targeting exon 9 of fIL6ST. In some embodiments, the CRISPR gene-editing system for the treatment of DDD comprises one or more sgRNAs targeting exon 10 of fIL6ST. In some embodiments, the CRISPR gene-editing system for the treatment of DDD comprises one or more sgRNAs targeting exon 11 of fIL6ST In some embodiments, the CRISPR gene-editing system for the treatment of DDD comprises one or more sgRNAs targeting exon 12 of fIL6ST. In some embodiments, the CRISPR gene-editing system for the treatment of DDD comprises one or more sgRNAs targeting exon 13 of fIL6ST. In some embodiments, the CRISPR gene-editing system for the treatment of DDD comprises one or more sgRNAs targeting exon 14 of fIL6ST. In some embodiments, the CRISPR gene-editing system for the treatment of DDD comprises one or more sgRNAs targeting exon 15 of fIL6ST. In some embodiments, the CRISPR gene-editing system for the treatment of DDD comprises one or more sgRNAs targeting exon 16 of fIL6ST. In some embodiments, the CRISPR gene-editing system for the treatment of DDD comprises one or more sgRNAs targeting exon 17 of fIL6ST. In some embodiments, the CRISPR gene-editing system for the treatment of DDD comprises one or more sgRNAs targeting exon 18 of fIL6ST. In some embodiments, the CRISPR gene-editing system for the treatment of DDD comprises one or more sgRNAs targeting exon 19 of fIL6ST. In some embodiments, the CRISPR gene-editing system for the treatment of DDD comprises one or more sgRNAs targeting exon 20 of fIL6ST. In some embodiments, the CRISPR gene-editing system for the treatment of DDD comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TNFRSF1A. In some embodiments, the CRISPR gene-editing system for the treatment of DDD comprises one or more sgRNAs targeting hTNFRSF1A. In some embodiments, the CRISPR gene-editing system for the treatment of DDD comprises one or more sgRNAs targeting cTNFRSF1A. In some embodiments, the CRISPR gene-editing system for the treatment of DDD comprises one or more sgRNAs targeting eTNFRSF1A. In some embodiments, the CRISPR gene-editing system for the treatment of DDD comprises one or more sgRNAs targeting fTNFRSF1A. In some embodiments, the CRISPR gene-editing system for the treatment of DDD comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTNFRSF1A. In some embodiments, the CRISPR gene-editing system for the treatment of DDD comprises one or more sgRNAs targeting exon 2 of hTNFRSF1A. In some embodiments, the CRISPR gene-editing system for the treatment of DDD comprises one or more sgRNAs targeting exon 3 of hTNFRSF1A. In some embodiments, the CRISPR gene-editing system for the treatment of DDD comprises one or more sgRNAs targeting exon 4 of hTNFRSF1A. In some embodiments, the CRISPR gene-editing system for the treatment of DDD comprises one or more sgRNAs targeting exon 5 of hTNFRSF1A. In some embodiments, the CRISPR gene-editing system for the treatment of DDD comprises one or more sgRNAs targeting exon 6 of hTNFRSF1A. In some embodiments, the CRISPR gene-editing system for the treatment of DDD comprises one or more sgRNAs targeting exon 7 of hTNFRSF1A. In some embodiments, the CRISPR gene-editing system for the treatment of DDD comprises one or more sgRNAs targeting exon 8 of hTNFRSF1A. In some embodiments, the CRISPR gene-editing system for the treatment of DDD comprises one or more sgRNAs targeting exon 9 of hTNFRSF1A. In some embodiments, the CRISPR gene-editing system for the treatment of DDD comprises one or more sgRNAs targeting exon 10 of hTNFRSF1A. In some embodiments, the CRISPR gene-editing system for the treatment of DDD comprises one or more sgRNAs targeting exon 11 of hTNFRSF1A. In some embodiments, the CRISPR gene-editing system for the treatment of DDD comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTNFRSF1A. In some embodiments, the CRISPR gene-editing system for the treatment of DDD comprises one or more sgRNAs targeting exon 2 of cTNFRSF1A. In some embodiments, the CRISPR gene-editing system for the treatment of DDD comprises one or more sgRNAs targeting exon 3 of cTNFRSF1A. In some embodiments, the CRISPR gene-editing system for the treatment of DDD comprises one or more sgRNAs targeting exon 4 of cTNFRSF1A. In some embodiments, the CRISPR gene-editing system for the treatment of DDD comprises one or more sgRNAs targeting exon 5 of cTNFRSF1A. In some embodiments, the CRISPR gene-editing system for the treatment of DDD comprises one or more sgRNAs targeting exon 6 of cTNFRSF1A. In some embodiments, the CRISPR gene-editing system for the treatment of DDD comprises one or more sgRNAs targeting exon 7 of cTNFRSF1A. In some embodiments, the CRISPR gene-editing system for the treatment of DDD comprises one or more sgRNAs targeting exon 8 of cTNFRSF1A. In some embodiments, the CRISPR gene-editing system for the treatment of DDD comprises one or more sgRNAs targeting exon 9 of cTNFRSF1A. In some embodiments, the CRISPR gene-editing system for the treatment of DDD comprises one or more sgRNAs targeting exon 10 of cTNFRSF1A. In some embodiments, the CRISPR gene-editing system for the treatment of DDD comprises one or more sgRNAs targeting exon 11 of cTNFRSF1A. In some embodiments, the CRISPR gene-editing system for the treatment of DDD comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTNFRSF1A. In some embodiments, the CRISPR gene-editing system for the treatment of DDD comprises one or more sgRNAs targeting exon 2 of eTNFRSF1A. In some embodiments, the CRISPR gene-editing system for the treatment of DDD comprises one or more sgRNAs targeting exon 3 of eTNFRSF1A. In some embodiments, the CRISPR gene-editing system for the treatment of DDD comprises one or more sgRNAs targeting exon 4 of eTNFRSF1A. In some embodiments, the CRISPR gene-editing system for the treatment of DDD comprises one or more sgRNAs targeting exon 5 of eTNFRSF1A. In some embodiments, the CRISPR gene-editing system for the treatment of DDD comprises one or more sgRNAs targeting exon 6 of eTNFRSF1A. In some embodiments, the CRISPR gene-editing system for the treatment of DDD comprises one or more sgRNAs targeting exon 7 of eTNFRSF1A. In some embodiments, the CRISPR gene-editing system for the treatment of DDD comprises one or more sgRNAs targeting exon 8 of eTNFRSF1A. In some embodiments, the CRISPR gene-editing system for the treatment of DDD comprises one or more sgRNAs targeting exon 9 of eTNFRSF1A. In some embodiments, the CRISPR gene-editing system for the treatment of DDD comprises one or more sgRNAs targeting exon 10 of eTNFRSF1A. In some embodiments, the CRISPR gene-editing system for the treatment of DDD comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTNFRSF1A. In some embodiments, the CRISPR gene-editing system for the treatment of DDD comprises one or more sgRNAs targeting exon 2 of fTNFRSF1A. In some embodiments, the CRISPR gene-editing system for the treatment of DDD comprises one or more sgRNAs targeting exon 3 of fTNFRSF1A. In some embodiments, the CRISPR gene-editing system for the treatment of DDD comprises one or more sgRNAs targeting exon 4 of fTNFRSF1A. In some embodiments, the CRISPR gene-editing system for the treatment of DDD comprises one or more sgRNAs targeting exon 5 of fTNFRSF1A. In some embodiments, the CRISPR gene-editing system for the treatment of DDD comprises one or more sgRNAs targeting exon 6 of fTNFRSF1A. In some embodiments, the CRISPR gene-editing system for the treatment of DDD comprises one or more sgRNAs targeting exon 7 of fTNFRSF1A. In some embodiments, the CRISPR gene-editing system for the treatment of DDD comprises one or more sgRNAs targeting exon 8 of fTNFRSF1A. In some embodiments, the CRISPR gene-editing system for the treatment of DDD comprises one or more sgRNAs targeting exon 9 of fTNFRSF1A. In some embodiments, the CRISPR gene-editing system for the treatment of DDD comprises one or more sgRNAs targeting exon 10 of fTNFRSF1A. In some embodiments, the CRISPR gene-editing system for the treatment of DDD comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TNFRSF1B. In some embodiments, the CRISPR gene-editing system for the treatment of DDD comprises one or more sgRNAs targeting hTNFRSF1B. In some embodiments, the CRISPR gene-editing system for the treatment of DDD comprises one or more sgRNAs targeting cTNFRSF1B. In some embodiments, the CRISPR gene-editing system for the treatment of DDD comprises one or more sgRNAs targeting eTNFRSF1B. In some embodiments, the CRISPR gene-editing system for the treatment of DDD comprises one or more sgRNAs targeting fTNFRSF1B. In some embodiments, the CRISPR gene-editing system for the treatment of DDD comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTNFRSF1B. In some embodiments, the CRISPR gene-editing system for the treatment of DDD comprises one or more sgRNAs targeting exon 2 of hTNFRSF1B. In some embodiments, the CRISPR gene-editing system for the treatment of DDD comprises one or more sgRNAs targeting exon 3 of hTNFRSF1B. In some embodiments, the CRISPR gene-editing system for the treatment of DDD comprises one or more sgRNAs targeting exon 4 of hTNFRSF1B. In some embodiments, the CRISPR gene-editing system for the treatment of DDD comprises one or more sgRNAs targeting exon 5 of hTNFRSF1B. In some embodiments, the CRISPR gene-editing system for the treatment of DDD comprises one or more sgRNAs targeting exon 6 of hTNFRSF1B. In some embodiments, the CRISPR gene-editing system for the treatment of DDD comprises one or more sgRNAs targeting exon 7 of hTNFRSF1B. In some embodiments, the CRISPR gene-editing system for the treatment of DDD comprises one or more sgRNAs targeting exon 8 of hTNFRSF1B. In some embodiments, the CRISPR gene-editing system for the treatment of DDD comprises one or more sgRNAs targeting exon 9 of hTNFRSF1B. In some embodiments, the CRISPR gene-editing system for the treatment of DDD comprises one or more sgRNAs targeting exon 10 of hTNFRSF1B. In some embodiments, the CRISPR gene-editing system for the treatment of DDD comprises one or more sgRNAs targeting exon 11 of hTNFRSF1B In some embodiments, the CRISPR gene-editing system for the treatment of DDD comprises one or more sgRNAs targeting exon 12 of hTNFRSF1B. In some embodiments, the CRISPR gene-editing system for the treatment of DDD comprises one or more sgRNAs targeting exon 13 of hTNFRSF1B. In some embodiments, the CRISPR gene-editing system for the treatment of DDD comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTNFRSF1B. In some embodiments, the CRISPR gene-editing system for the treatment of DDD comprises one or more sgRNAs targeting exon 2 of cTNFRSF1B. In some embodiments, the CRISPR gene-editing system for the treatment of DDD comprises one or more sgRNAs targeting exon 3 of cTNFRSF1B. In some embodiments, the CRISPR gene-editing system for the treatment of DDD comprises one or more sgRNAs targeting exon 4 of cTNFRSF1B. In some embodiments, the CRISPR gene-editing system for the treatment of DDD comprises one or more sgRNAs targeting exon 5 of cTNFRSF1B. In some embodiments, the CRISPR gene-editing system for the treatment of DDD comprises one or more sgRNAs targeting exon 6 of cTNFRSF1B. In some embodiments, the CRISPR gene-editing system for the treatment of DDD comprises one or more sgRNAs targeting exon 7 of cTNFRSF1B. In some embodiments, the CRISPR gene-editing system for the treatment of DDD comprises one or more sgRNAs targeting exon 8 of cTNFRSF1B. In some embodiments, the CRISPR gene-editing system for the treatment of DDD comprises one or more sgRNAs targeting exon 9 of cTNFRSF1B. In some embodiments, the CRISPR gene-editing system for the treatment of DDD comprises one or more sgRNAs targeting exon 10 of cTNFRSF1B. In some embodiments, the CRISPR gene-editing system for the treatment of DDD comprises one or more sgRNAs targeting exon 11 of cTNFRSF1B. In some embodiments, the CRISPR gene-editing system for the treatment of DDD comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTNFRSF1B. In some embodiments, the CRISPR gene-editing system for the treatment of DDD comprises one or more sgRNAs targeting exon 2 of eTNFRSF1B. In some embodiments, the CRISPR gene-editing system for the treatment of DDD comprises one or more sgRNAs targeting exon 3 of eTNFRSF1B. In some embodiments, the CRISPR gene-editing system for the treatment of DDD comprises one or more sgRNAs targeting exon 4 of eTNFRSF1B. In some embodiments, the CRISPR gene-editing system for the treatment of DDD comprises one or more sgRNAs targeting exon 5 of eTNFRSF1B. In some embodiments, the CRISPR gene-editing system for the treatment of DDD comprises one or more sgRNAs targeting exon 6 of eTNFRSF1B. In some embodiments, the CRISPR gene-editing system for the treatment of DDD comprises one or more sgRNAs targeting exon 7 of eTNFRSF1B. In some embodiments, the CRISPR gene-editing system for the treatment of DDD comprises one or more sgRNAs targeting exon 8 of eTNFRSF1B. In some embodiments, the CRISPR gene-editing system for the treatment of DDD comprises one or more sgRNAs targeting exon 9 of eTNFRSF1B. In some embodiments, the CRISPR gene-editing system for the treatment of DDD comprises one or more sgRNAs targeting exon 10 of eTNFRSF1B. In some embodiments, the CRISPR gene-editing system for the treatment of DDD comprises one or more sgRNAs targeting exon 11 of eTNFRSF1B. In some embodiments, the CRISPR gene-editing system for the treatment of DDD comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTNFRSF1B. In some embodiments, the CRISPR gene-editing system for the treatment of DDD comprises one or more sgRNAs targeting exon 2 of fTNFRSF1B. In some embodiments, the CRISPR gene-editing system for the treatment of DDD comprises one or more sgRNAs targeting exon 3 of fTNFRSF1B. In some embodiments, the CRISPR gene-editing system for the treatment of DDD comprises one or more sgRNAs targeting exon 4 of fTNFRSF1B. In some embodiments, the CRISPR gene-editing system for the treatment of DDD comprises one or more sgRNAs targeting exon 5 of fTNFRSF1B. In some embodiments, the CRISPR gene-editing system for the treatment of DDD comprises one or more sgRNAs targeting exon 6 of fTNFRSF1B. In some embodiments, the CRISPR gene-editing system for the treatment of DDD comprises one or more sgRNAs targeting exon 7 of fTNFRSF1B. In some embodiments, the CRISPR gene-editing system for the treatment of DDD comprises one or more sgRNAs targeting exon 8 of fTNFRSF1B. In some embodiments, the CRISPR gene-editing system for the treatment of DDD comprises one or more sgRNAs targeting exon 9 of fTNFRSF1B. In some embodiments, the CRISPR gene-editing system for the treatment of DDD comprises one or more sgRNAs targeting exon 10 of fTNFRSF1B. In some embodiments, the CRISPR gene-editing system for the treatment of DDD comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TNFRSF3. In some embodiments, the CRISPR gene-editing system for the treatment of DDD comprises one or more sgRNAs targeting hTNFRSF3. In some embodiments, the CRISPR gene-editing system for the treatment of DDD comprises one or more sgRNAs targeting cTNFRSF3. In some embodiments, the CRISPR gene-editing system for the treatment of DDD comprises one or more sgRNAs targeting eTNFRSF3. In some embodiments, the CRISPR gene-editing system for the treatment of DDD comprises one or more sgRNAs targeting fTNFRSF3. In some embodiments, the CRISPR gene-editing system for the treatment of DDD comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTNFRSF3. In some embodiments, the CRISPR gene-editing system for the treatment of DDD comprises one or more sgRNAs targeting exon 2 of hTNFRSF3. In some embodiments, the CRISPR gene-editing system for the treatment of DDD comprises one or more sgRNAs targeting exon 3 of hTNFRSF3. In some embodiments, the CRISPR gene-editing system for the treatment of DDD comprises one or more sgRNAs targeting exon 4 of hTNFRSF3. In some embodiments, the CRISPR gene-editing system for the treatment of DDD comprises one or more sgRNAs targeting exon 5 of hTNFRSF3. In some embodiments, the CRISPR gene-editing system for the treatment of DDD comprises one or more sgRNAs targeting exon 6 of hTNFRSF3. In some embodiments, the CRISPR gene-editing system for the treatment of DDD comprises one or more sgRNAs targeting exon 7 of hTNFRSF3. In some embodiments, the CRISPR gene-editing system for the treatment of DDD comprises one or more sgRNAs targeting exon 8 of hTNFRSF3. In some embodiments, the CRISPR gene-editing system for the treatment of DDD comprises one or more sgRNAs targeting exon 9 of hTNFRSF3. In some embodiments, the CRISPR gene-editing system for the treatment of DDD comprises one or more sgRNAs targeting exon 10 of hTNFRSF3. In some embodiments, the CRISPR gene-editing system for the treatment of DDD comprises one or more sgRNAs targeting exon 11 of hTNFRSF3. In some embodiments, the CRISPR gene-editing system for the treatment of DDD comprises one or more sgRNAs targeting exon 12 of hTNFRSF3. In some embodiments, the CRISPR gene-editing system for the treatment of DDD comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTNFRSF3. In some embodiments, the CRISPR gene-editing system for the treatment of DDD comprises one or more sgRNAs targeting exon 2 of cTNFRSF3. In some embodiments, the CRISPR gene-editing system for the treatment of DDD comprises one or more sgRNAs targeting exon 3 of cTNFRSF3. In some embodiments, the CRISPR gene-editing system for the treatment of DDD comprises one or more sgRNAs targeting exon 4 of cTNFRSF3. In some embodiments, the CRISPR gene-editing system for the treatment of DDD comprises one or more sgRNAs targeting exon 5 of cTNFRSF3. In some embodiments, the CRISPR gene-editing system for the treatment of DDD comprises one or more sgRNAs targeting exon 6 of cTNFRSF3. In some embodiments, the CRISPR gene-editing system for the treatment of DDD comprises one or more sgRNAs targeting exon 7 of cTNFRSF3. In some embodiments, the CRISPR gene-editing system for the treatment of DDD comprises one or more sgRNAs targeting exon 8 of cTNFRSF3. In some embodiments, the CRISPR gene-editing system for the treatment of DDD comprises one or more sgRNAs targeting exon 9 of cTNFRSF3. In some embodiments, the CRISPR gene-editing system for the treatment of DDD comprises one or more sgRNAs targeting exon 10 of cTNFRSF3. In some embodiments, the CRISPR gene-editing system for the treatment of DDD comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTNFRSF3. In some embodiments, the CRISPR gene-editing system for the treatment of DDD comprises one or more sgRNAs targeting exon 2 of eTNFRSF3. In some embodiments, the CRISPR gene-editing system for the treatment of DDD comprises one or more sgRNAs targeting exon 3 of eTNFRSF3. In some embodiments, the CRISPR gene-editing system for the treatment of DDD comprises one or more sgRNAs targeting exon 4 of eTNFRSF3. In some embodiments, the CRISPR gene-editing system for the treatment of DDD comprises one or more sgRNAs targeting exon 5 of eTNFRSF3. In some embodiments, the CRISPR gene-editing system for the treatment of DDD comprises one or more sgRNAs targeting exon 6 of eTNFRSF3. In some embodiments, the CRISPR gene-editing system for the treatment of DDD comprises one or more sgRNAs targeting exon 7 of eTNFRSF3. In some embodiments, the CRISPR gene-editing system for the treatment of DDD comprises one or more sgRNAs targeting exon 8 of eTNFRSF3. In some embodiments, the CRISPR gene-editing system for the treatment of DDD comprises one or more sgRNAs targeting exon 9 of eTNFRSF3. In some embodiments, the CRISPR gene-editing system for the treatment of DDD comprises one or more sgRNAs targeting exon 10 of eTNFRSF3. In some embodiments, the CRISPR gene-editing system for the treatment of DDD comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTNFRSF3. In some embodiments, the CRISPR gene-editing system for the treatment of DDD comprises one or more sgRNAs targeting exon 2 of fTNFRSF3. In some embodiments, the CRISPR gene-editing system for the treatment of DDD comprises one or more sgRNAs targeting exon 3 of fTNFRSF3. In some embodiments, the CRISPR gene-editing system for the treatment of DDD comprises one or more sgRNAs targeting exon 4 of fTNFRSF3. In some embodiments, the CRISPR gene-editing system for the treatment of DDD comprises one or more sgRNAs targeting exon 5 of fTNFRSF3. In some embodiments, the CRISPR gene-editing system for the treatment of DDD comprises one or more sgRNAs targeting exon 6 of fTNFRSF3. In some embodiments, the CRISPR gene-editing system for the treatment of DDD comprises one or more sgRNAs targeting exon 7 of fTNFRSF3. In some embodiments, the CRISPR gene-editing system for the treatment of DDD comprises one or more sgRNAs targeting exon 8 of fTNFRSF3. In some embodiments, the CRISPR gene-editing system for the treatment of DDD comprises one or more sgRNAs targeting exon 9 of fTNFRSF3. In some embodiments, the CRISPR gene-editing system for the treatment of DDD comprises one or more sgRNAs targeting exon 10 of fTNFRSF3. In some embodiments, the CRISPR gene-editing system for the treatment of DDD comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TNFRSF4. In some embodiments, the CRISPR gene-editing system for the treatment of DDD comprises one or more sgRNAs targeting hTNFRSF4. In some embodiments, the CRISPR gene-editing system for the treatment of DDD comprises one or more sgRNAs targeting cTNFRSF4. In some embodiments, the CRISPR gene-editing system for the treatment of DDD comprises one or more sgRNAs targeting eTNFRSF4. In some embodiments, the CRISPR gene-editing system for the treatment of DDD comprises one or more sgRNAs targeting fTNFRSF4. In some embodiments, the CRISPR gene-editing system for the treatment of DDD comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTNFRSF4. In some embodiments, the CRISPR gene-editing system for the treatment of DDD comprises one or more sgRNAs targeting exon 2 of hTNFRSF4. In some embodiments, the CRISPR gene-editing system for the treatment of DDD comprises one or more sgRNAs targeting exon 3 of hTNFRSF4. In some embodiments, the CRISPR gene-editing system for the treatment of DDD comprises one or more sgRNAs targeting exon 4 of hTNFRSF4. In some embodiments, the CRISPR gene-editing system for the treatment of DDD comprises one or more sgRNAs targeting exon 5 of hTNFRSF4. In some embodiments, the CRISPR gene-editing system for the treatment of DDD comprises one or more sgRNAs targeting exon 6 of hTNFRSF4. In some embodiments, the CRISPR gene-editing system for the treatment of DDD comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTNFRSF4. In some embodiments, the CRISPR gene-editing system for the treatment of DDD comprises one or more sgRNAs targeting exon 2 of cTNFRSF4. In some embodiments, the CRISPR gene-editing system for the treatment of DDD comprises one or more sgRNAs targeting exon 3 of cTNFRSF4. In some embodiments, the CRISPR gene-editing system for the treatment of DDD comprises one or more sgRNAs targeting exon 4 of cTNFRSF4. In some embodiments, the CRISPR gene-editing system for the treatment of DDD comprises one or more sgRNAs targeting exon 5 of cTNFRSF4. In some embodiments, the CRISPR gene-editing system for the treatment of DDD comprises one or more sgRNAs targeting exon 6 of cTNFRSF4. In some embodiments, the CRISPR gene-editing system for the treatment of DDD comprises one or more sgRNAs targeting exon 7 of cTNFRSF4. In some embodiments, the CRISPR gene-editing system for the treatment of DDD comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTNFRSF4. In some embodiments, the CRISPR gene-editing system for the treatment of DDD comprises one or more sgRNAs targeting exon 2 of eTNFRSF4. In some embodiments, the CRISPR gene-editing system for the treatment of DDD comprises one or more sgRNAs targeting exon 3 of eTNFRSF4. In some embodiments, the CRISPR gene-editing system for the treatment of DDD comprises one or more sgRNAs targeting exon 4 of eTNFRSF4. In some embodiments, the CRISPR gene-editing system for the treatment of DDD comprises one or more sgRNAs targeting exon 5 of eTNFRSF4. In some embodiments, the CRISPR gene-editing system for the treatment of DDD comprises one or more sgRNAs targeting exon 6 of eTNFRSF4. In some embodiments, the CRISPR gene-editing system for the treatment of DDD comprises one or more sgRNAs targeting exon 7 of eTNFRSF4. In some embodiments, the CRISPR gene-editing system for the treatment of DDD comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTNFRSF4. In some embodiments, the CRISPR gene-editing system for the treatment of DDD comprises one or more sgRNAs targeting exon 2 of fTNFRSF4. In some embodiments, the CRISPR gene-editing system for the treatment of DDD comprises one or more sgRNAs targeting exon 3 of fTNFRSF4. In some embodiments, the CRISPR gene-editing system for the treatment of DDD comprises one or more sgRNAs targeting exon 4 of fTNFRSF4. In some embodiments, the CRISPR gene-editing system for the treatment of DDD comprises one or more sgRNAs targeting exon 5 of fTNFRSF4. In some embodiments, the CRISPR gene-editing system for the treatment of DDD comprises one or more sgRNAs targeting exon 6 of fTNFRSF4. In some embodiments, the CRISPR gene-editing system for the treatment of DDD comprises one or more sgRNAs targeting exon 7 of fTNFRSF4. In some embodiments, the CRISPR gene-editing system for the treatment of DDD comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting TNFRSF11A. In some embodiments, the CRISPR gene-editing system for the treatment of DDD comprises one or more sgRNAs targeting hTNFRSF11A. In some embodiments, the CRISPR gene-editing system for the treatment of DDD comprises one or more sgRNAs targeting cTNFRSF11A. In some embodiments, the CRISPR gene-editing system for the treatment of DDD comprises one or more sgRNAs targeting eTNFRSF11A. In some embodiments, the CRISPR gene-editing system for the treatment of DDD comprises one or more sgRNAs targeting fTNFRSF11A. In some embodiments, the CRISPR gene-editing system for the treatment of DDD comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of hTNFRSF11A. In some embodiments, the CRISPR gene-editing system for the treatment of DDD comprises one or more sgRNAs targeting exon 2 of hTNFRSF11A. In some embodiments, the CRISPR gene-editing system for the treatment of DDD comprises one or more sgRNAs targeting exon 3 of hTNFRSF11A. In some embodiments, the CRISPR gene-editing system for the treatment of DDD comprises one or more sgRNAs targeting exon 4 of hTNFRSF11A. In some embodiments, the CRISPR gene-editing system for the treatment of DDD comprises one or more sgRNAs targeting exon 5 of hTNFRSF11A. In some embodiments, the CRISPR gene-editing system for the treatment of DDD comprises one or more sgRNAs targeting exon 6 of hTNFRSF11A. In some embodiments, the CRISPR gene-editing system for the treatment of DDD comprises one or more sgRNAs targeting exon 7 of hTNFRSF11A. In some embodiments, the CRISPR gene-editing system for the treatment of DDD comprises one or more sgRNAs targeting exon 8 of hTNFRSF11A. In some embodiments, the CRISPR gene-editing system for the treatment of DDD comprises one or more sgRNAs targeting exon 9 of hTNFRSF11A. In some embodiments, the CRISPR gene-editing system for the treatment of DDD comprises one or more sgRNAs targeting exon 10 of hTNFRSF11A. In some embodiments, the CRISPR gene-editing system for the treatment of DDD comprises one or more sgRNAs targeting exon 11 of hTNFRSF11A. In some embodiments, the CRISPR gene-editing system for the treatment of DDD comprises one or more sgRNAs targeting exon 12 of hTNFRSF11A. In some embodiments, the CRISPR gene-editing system for the treatment of DDD comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of cTNFRSF11A. In some embodiments, the CRISPR gene-editing system for the treatment of DDD comprises one or more sgRNAs targeting exon 2 of cTNFRSF11A. In some embodiments, the CRISPR gene-editing system for the treatment of DDD comprises one or more sgRNAs targeting exon 3 of cTNFRSF11A. In some embodiments, the CRISPR gene-editing system for the treatment of DDD comprises one or more sgRNAs targeting exon 4 of cTNFRSF11A. In some embodiments, the CRISPR gene-editing system for the treatment of DDD comprises one or more sgRNAs targeting exon 5 of cTNFRSF11A. In some embodiments, the CRISPR gene-editing system for the treatment of DDD comprises one or more sgRNAs targeting exon 6 of cTNFRSF11A. In some embodiments, the CRISPR gene-editing system for the treatment of DDD comprises one or more sgRNAs targeting exon 7 of cTNFRSF11A. In some embodiments, the CRISPR gene-editing system for the treatment of DDD comprises one or more sgRNAs targeting exon 8 of cTNFRSF11A. In some embodiments, the CRISPR gene-editing system for the treatment of DDD comprises one or more sgRNAs targeting exon 9 of cTNFRSF11A. In some embodiments, the CRISPR gene-editing system for the treatment of DDD comprises one or more sgRNAs targeting exon 10 of cTNFRSF11A. In some embodiments, the CRISPR gene-editing system for the treatment of DDD comprises one or more sgRNAs targeting exon 11 of cTNFRSF11A. In some embodiments, the CRISPR gene-editing system for the treatment of DDD comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of eTNFRSF11A. In some embodiments, the CRISPR gene-editing system for the treatment of DDD comprises one or more sgRNAs targeting exon 2 of eTNFRSF11A. In some embodiments, the CRISPR gene-editing system for the treatment of DDD comprises one or more sgRNAs targeting exon 3 of eTNFRSF11A. In some embodiments, the CRISPR gene-editing system for the treatment of DDD comprises one or more sgRNAs targeting exon 4 of eTNFRSF11A. In some embodiments, the CRISPR gene-editing system for the treatment of DDD comprises one or more sgRNAs targeting exon 5 of eTNFRSF11A. In some embodiments, the CRISPR gene-editing system for the treatment of DDD comprises one or more sgRNAs targeting exon 6 of eTNFRSF11A. In some embodiments, the CRISPR gene-editing system for the treatment of DDD comprises one or more sgRNAs targeting exon 7 of eTNFRSF11A. In some embodiments, the CRISPR gene-editing system for the treatment of DDD comprises one or more sgRNAs targeting exon 8 of eTNFRSF11A. In some embodiments, the CRISPR gene-editing system for the treatment of DDD comprises one or more sgRNAs targeting exon 9 of eTNFRSF11A. In some embodiments, the CRISPR gene-editing system for the treatment of DDD comprises one or more sgRNAs targeting exon 10 of eTNFRSF11A. In some embodiments, the CRISPR gene-editing system for the treatment of DDD comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of DDD comprises one or more sgRNAs targeting exon 1 of fTNFRSF11A. In some embodiments, the CRISPR gene-editing system for the treatment of DDD comprises one or more sgRNAs targeting exon 2 of fTNFRSF11A. In some embodiments, the CRISPR gene-editing system for the treatment of DDD comprises one or more sgRNAs targeting exon 3 of fTNFRSF11A. In some embodiments, the CRISPR gene-editing system for the treatment of DDD comprises one or more sgRNAs targeting exon 4 of fTNFRSF11A. In some embodiments, the CRISPR gene-editing system for the treatment of DDD comprises one or more sgRNAs targeting exon 5 of fTNFRSF11A. In some embodiments, the CRISPR gene-editing system for the treatment of DDD comprises one or more sgRNAs targeting exon 6 of fTNFRSF11A. In some embodiments, the CRISPR gene-editing system for the treatment of DDD comprises one or more sgRNAs targeting exon 7 of fTNFRSF11A. In some embodiments, the CRISPR gene-editing system for the treatment of DDD comprises one or more sgRNAs targeting exon 8 of fTNFRSF11A. In some embodiments, the CRISPR gene-editing system for the treatment of DDD comprises one or more sgRNAs targeting exon 9 of fTNFRSF11A. In some embodiments, the CRISPR gene-editing system for the treatment of DDD comprises one or more sgRNAs targeting exon 10 of fTNFRSF11A. In some embodiments, the CRISPR gene-editing system for the treatment of DDD comprises one or more sgRNAs targeting exon 11 of fTNFRSF11A. In some embodiments, the CRISPR gene-editing system for the treatment of DDD comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### D. IDD

In one aspect, the present disclosure encompasses treatments for internal disc disruption (IDD). In some embodiments, the IDD treatment comprises a therapeutically effective amount of a Clustered Regularly Interspaced Short Palindromic Repeats (CRISPR) gene-editing system, the system comprising: (i) a CRISPR Associated (Cas) protein; and (ii) at least one guide RNA targeting IL1R1. In some embodiments, the IDD treatment comprises a CRISPR gene-editing system targeting hIL1R1. In some embodiments, the IDD treatment comprises a CRISPR gene-editing system targeting cIL1RT. In some embodiments, the IDD treatment comprises a CRISP gene-editing system R targeting eIL1R1. In some embodiments, the IDD treatment comprises a CRISPR gene-editing system targeting fIL1R1. In some embodiments, the CRISPR gene-editing system for the treatment of IDD comprising one or more sgRNAs targeting an exon of IL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hIL1R1. In some embodiments, the CRISPR gene-editing system for the treatment of IDD comprises one or more sgRNAs targeting exon 2 of hIL1R1. In some embodiments, the CRISPR gene-editing system for the treatment of IDD comprises one or more sgRNAs targeting exon 3 of hIL1R1. In some embodiments, the CRISPR gene-editing system for the treatment of IDD comprises one or more sgRNAs targeting exon 4 of hIL1R1. In some embodiments, the CRISPR gene-editing system for the treatment of IDD comprises one or more sgRNAs targeting exon 5 of hIL1R1. In some embodiments, the CRISPR gene-editing system for the treatment of IDD comprises one or more sgRNAs targeting exon 6 of hIL1R1. In some embodiments, the CRISPR gene-editing system for the treatment of IDD comprises one or more sgRNAs targeting exon 7 of hIL1R1. In some embodiments, the CRISPR gene-editing system for the treatment of IDD comprises one or more sgRNAs targeting exon 8 of hIL1R1. In some embodiments, the CRISPR gene-editing system for the treatment of IDD comprises one or more sgRNAs targeting exon 9 of hIL1R1. In some embodiments, the CRISPR gene-editing system for the treatment of IDD comprises one or more sgRNAs targeting exon 10 of hIL1R1. In some embodiments, the CRISPR gene-editing system for the treatment of IDD comprises one or more sgRNAs targeting exon 11 of hIL1R1. In some embodiments, the CRISPR gene-editing system for the treatment of IDD comprises one or more sgRNAs targeting exon 12 of hIL1R1. In some embodiments, the CRISPR gene-editing system for the treatment of IDD comprises one or more sgRNAs targeting exon 13 of hIL1R1. In some embodiments, the CRISPR gene-editing system for the treatment of IDD comprises one or more sgRNAs targeting exon 14 of hIL1R1. In some embodiments, the CRISPR gene-editing system for the treatment of IDD comprises one or more sgRNAs targeting exon 15 of hIL1R1. In some embodiments, the CRISPR gene-editing system for the treatment of IDD comprises one or more sgRNAs targeting exon 16 of hIL1R1. In some embodiments, the CRISPR gene-editing system for the treatment of IDD comprises one or more sgRNAs targeting exon 17 of hIL1R1. In some embodiments, the CRISPR gene-editing system for the treatment of IDD comprises one or more sgRNAs targeting exon 18 of hIL1R1. In some embodiments, the CRISPR gene-editing system for the treatment of IDD comprises one or more sgRNAs targeting exon 19 of hIL1R1. In some embodiments, the CRISPR gene-editing system for the treatment of IDD comprises one or more sgRNAs targeting exon 20 of hIL1R1. In some embodiments, the CRISPR gene-editing system for the treatment of IDD comprises one or more sgRNAs targeting exon 21 of hIL1R1. In some embodiments, the CRISPR gene-editing system for the treatment of IDD comprising one or more sgRNAs targeting an exon of hIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cIL1R1. In some embodiments, the CRISPR gene-editing system for the treatment of IDD comprises one or more sgRNAs targeting exon 2 of cIL1R1. In some embodiments, the CRISPR gene-editing system for the treatment of IDD comprises one or more sgRNAs targeting exon 3 of cIL1R1. In some embodiments, the CRISPR gene-editing system for the treatment of IDD comprises one or more sgRNAs targeting exon 4 of cIL1R1. In some embodiments, the CRISPR gene-editing system for the treatment of IDD comprises one or more sgRNAs targeting exon 5 of cIL1R1. In some embodiments, the CRISPR gene-editing system for the treatment of IDD comprises one or more sgRNAs targeting exon 6 of cIL1R1. In some embodiments, the CRISPR gene-editing system for the treatment of IDD comprises one or more sgRNAs targeting exon 7 of cIL1R1. In some embodiments, the CRISPR gene-editing system for the treatment of IDD comprises one or more sgRNAs targeting exon 8 of cIL1R1. In some embodiments, the CRISPR gene-editing system for the treatment of IDD comprises one or more sgRNAs targeting exon 9 of cIL1R1. In some embodiments, the CRISPR gene-editing system for the treatment of IDD comprises one or more sgRNAs targeting exon 10 of cIL1R1. In some embodiments, the CRISPR gene-editing system for the treatment of IDD comprises one or more sgRNAs targeting exon 11 of cIL1R1. In some embodiments, the CRISPR gene-editing system for the treatment of IDD comprises one or more sgRNAs targeting exon 12 of cIL1R1. In some embodiments, the CRISPR gene-editing system for the treatment of IDD comprises one or more sgRNAs targeting exon 13 of cIL1R1. In some embodiments, the CRISPR gene-editing system for the treatment of IDD comprises one or more sgRNAs targeting exon 14 of cIL1R1. In some embodiments, the CRISPR gene-editing system for the treatment of IDD comprises one or more sgRNAs targeting exon 15 of cIL1R1. In some embodiments, the CRISPR gene-editing system for the treatment of IDD comprises one or more sgRNAs targeting exon 16 of cIL1R1. In some embodiments, the CRISPR gene-editing system for the treatment of IDD comprising one or more sgRNAs targeting an exon of cIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eIL1R1. In some embodiments, the CRISPR gene-editing system for the treatment of IDD comprises one or more sgRNAs targeting exon 2 of eIL1R1. In some embodiments, the CRISPR gene-editing system for the treatment of IDD comprises one or more sgRNAs targeting exon 3 of eIL1R1. In some embodiments, the CRISPR gene-editing system for the treatment of IDD comprises one or more sgRNAs targeting exon 4 of eIL1R1. In some embodiments, the CRISPR gene-editing system for the treatment of IDD comprises one or more sgRNAs targeting exon 5 of eIL1R1. In some embodiments, the CRISPR gene-editing system for the treatment of IDD comprises one or more sgRNAs targeting exon 6 of eIL1R1. In some embodiments, the CRISPR gene-editing system for the treatment of IDD comprises one or more sgRNAs targeting exon 7 of eIL1R1. In some embodiments, the CRISPR gene-editing system for the treatment of IDD comprises one or more sgRNAs targeting exon 8 of eIL1R1. In some embodiments, the CRISPR gene-editing system for the treatment of IDD comprises one or more sgRNAs targeting exon 9 of eIL1R1. In some embodiments, the CRISPR gene-editing system for the treatment of IDD comprises one or more sgRNAs targeting exon 10 of eIL1R1. In some embodiments, the CRISPR gene-editing system for the treatment of IDD comprises one or more sgRNAs targeting exon 11 of eIL1R1. In some embodiments, the CRISPR gene-editing system for the treatment of IDD comprises one or more sgRNAs targeting exon 12 of eIL1R1. In some embodiments, the CRISPR gene-editing system for the treatment of IDD comprises one or more sgRNAs targeting exon 13 of eIL1R1. In some embodiments, the CRISPR gene-editing system for the treatment of IDD comprises one or more sgRNAs targeting exon 14 of eIL1R1. In some embodiments, the CRISPR gene-editing system for the treatment of IDD comprises one or more sgRNAs targeting exon 15 of eIL1R1. In some embodiments, the CRISPR gene-editing system for the treatment of IDD comprising one or more sgRNAs targeting an exon of eIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fIL1R1. In some embodiments, the CRISPR gene-editing system for the treatment of IDD comprises one or more sgRNAs targeting exon 2 of fIL1R1. In some embodiments, the CRISPR gene-editing system for the treatment of IDD comprises one or more sgRNAs targeting exon 3 of fIL1R1. In some embodiments, the CRISPR gene-editing system for the treatment of IDD comprises one or more sgRNAs targeting exon 4 of fIL1R1. In some embodiments, the CRISPR gene-editing system for the treatment of IDD comprises one or more sgRNAs targeting exon 5 of fIL1R1. In some embodiments, the CRISPR gene-editing system for the treatment of IDD comprises one or more sgRNAs targeting exon 6 of fIL1R1. In some embodiments, the CRISPR gene-editing system for the treatment of IDD comprises one or more sgRNAs targeting exon 7 of fIL1R1. In some embodiments, the CRISPR gene-editing system for the treatment of IDD comprises one or more sgRNAs targeting exon 8 of fIL1R1. In some embodiments, the CRISPR gene-editing system for the treatment of IDD comprises one or more sgRNAs targeting exon 9 of fIL1R1. In some embodiments, the CRISPR gene-editing system for the treatment of IDD comprises one or more sgRNAs targeting exon 10 of fIL1R1. In some embodiments, the CRISPR gene-editing system for the treatment of IDD comprises one or more sgRNAs targeting exon 11 of fIL1R. In some embodiments, the CRISPR gene-editing system for the treatment of IDD comprises one or more sgRNAs targeting exon 12 of fIL1R1. In some embodiments, the CRISPR gene-editing system for the treatment of IDD comprises one or more sgRNAs targeting exon 13 of fIL1R1. In some embodiments, the CRISPR gene-editing system for the treatment of IDD comprises one or more sgRNAs targeting exon 14 of fIL1R1. In some embodiments, the CRISPR gene-editing system for the treatment of IDD comprises one or more sgRNAs targeting exon 15 of fIL1R. In some embodiments, the CRISPR gene-editing system for the treatment of IDD comprises one or more sgRNAs targeting exon 16 of fIL1R1. In some embodiments, the CRISPR gene-editing system for the treatment of IDD comprises one or more sgRNAs targeting exon 17 of fIL1R1. In some embodiments, the CRISPR gene-editing system for the treatment of IDD comprises one or more sgRNAs targeting exon 18 of fIL1R1. In some embodiments, the CRISPR gene-editing system for the treatment of IDD comprising one or more sgRNAs targeting an exon of fIL1R1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting IL1RAP. In some embodiments, the CRISPR gene-editing system for the treatment of IDD comprises one or more sgRNAs targeting hIL1RAP. In some embodiments, the CRISPR gene-editing system for the treatment of IDD comprises one or more sgRNAs targeting cIL1RAP. In some embodiments, the CRISPR gene-editing system for the treatment of IDD comprises one or more sgRNAs targeting eIL1RAP. In some embodiments, the CRISPR gene-editing system for the treatment of IDD comprises one or more sgRNAs targeting fIL1RAP. In some embodiments, the CRISPR gene-editing system for the treatment of IDD comprising one or more sgRNAs targeting an exon of IL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hIL1RAP. In some embodiments, the CRISPR gene-editing system for the treatment of IDD comprises one or more sgRNAs targeting exon 2 of hIL1RAP. In some embodiments, the CRISPR gene-editing system for the treatment of IDD comprises one or more sgRNAs targeting exon 3 of hIL1RAP. In some embodiments, the CRISPR gene-editing system for the treatment of IDD comprises one or more sgRNAs targeting exon 4 of hIL1RAP. In some embodiments, the CRISPR gene-editing system for the treatment of IDD comprises one or more sgRNAs targeting exon 5 of hIL1RAP. In some embodiments, the CRISPR gene-editing system for the treatment of IDD comprises one or more sgRNAs targeting exon 6 of hIL1RAP. In some embodiments, the CRISPR gene-editing system for the treatment of IDD comprises one or more sgRNAs targeting exon 7 of hIL1RAP. In some embodiments, the CRISPR gene-editing system for the treatment of IDD comprises one or more sgRNAs targeting exon 8 of hIL1RAP. In some embodiments, the CRISPR gene-editing system for the treatment of IDD comprises one or more sgRNAs targeting exon 9 of hIL1RAP. In some embodiments, the CRISPR gene-editing system for the treatment of IDD comprises one or more sgRNAs targeting exon 10 of hIL1RAP. In some embodiments, the CRISPR gene-editing system for the treatment of IDD comprises one or more sgRNAs targeting exon 11 of hIL1RAP. In some embodiments, the CRISPR gene-editing system for the treatment of IDD comprises one or more sgRNAs targeting exon 12 of hIL1RAP. In some embodiments, the CRISPR gene-editing system for the treatment of IDD comprises one or more sgRNAs targeting exon 13 of hIL1RAP. In some embodiments, the CRISPR gene-editing system for the treatment of IDD comprises one or more sgRNAs targeting exon 14 of hIL1RAP. In some embodiments, the CRISPR gene-editing system for the treatment of IDD comprises one or more sgRNAs targeting exon 15 of hIL1RAP. In some embodiments, the CRISPR gene-editing system for the treatment of IDD comprises one or more sgRNAs targeting exon 16 of hIL1RAP. In some embodiments, the CRISPR gene-editing system for the treatment of IDD comprises one or more sgRNAs targeting exon 17 of hIL1RAP. In some embodiments, the CRISPR gene-editing system for the treatment of IDD comprising one or more sgRNAs targeting an exon of hIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cIL1RAP. In some embodiments, the CRISPR gene-editing system for the treatment of IDD comprises one or more sgRNAs targeting exon 2 of cIL1RAP. In some embodiments, the CRISPR gene-editing system for the treatment of IDD comprises one or more sgRNAs targeting exon 3 of cIL1RAP. In some embodiments, the CRISPR gene-editing system for the treatment of IDD comprises one or more sgRNAs targeting exon 4 of cIL1RAP. In some embodiments, the CRISPR gene-editing system for the treatment of IDD comprises one or more sgRNAs targeting exon 5 of cIL1RAP. In some embodiments, the CRISPR gene-editing system for the treatment of IDD comprises one or more sgRNAs targeting exon 6 of cIL1RAP. In some embodiments, the CRISPR gene-editing system for the treatment of IDD comprises one or more sgRNAs targeting exon 7 of cIL1RAP. In some embodiments, the CRISPR gene-editing system for the treatment of IDD comprises one or more sgRNAs targeting exon 8 of cIL1RAP. In some embodiments, the CRISPR gene-editing system for the treatment of IDD comprises one or more sgRNAs targeting exon 9 of cIL1RAP. In some embodiments, the CRISPR gene-editing system for the treatment of IDD comprises one or more sgRNAs targeting exon 10 of cIL1RAP. In some embodiments, the CRISPR gene-editing system for the treatment of IDD comprises one or more sgRNAs targeting exon 11 of cIL1RAP. In some embodiments, the CRISPR gene-editing system for the treatment of IDD comprises one or more sgRNAs targeting exon 12 of cIL1RAP. In some embodiments, the CRISPR gene-editing system for the treatment of IDD comprises one or more sgRNAs targeting exon 13 of cIL1RAP. In some embodiments, the CRISPR gene-editing system for the treatment of IDD comprises one or more sgRNAs targeting exon 14 of cIL1RAP. In some embodiments, the CRISPR gene-editing system for the treatment of IDD comprises one or more sgRNAs targeting exon 15 of cIL1RAP. In some embodiments, the CRISPR gene-editing system for the treatment of IDD comprises one or more sgRNAs targeting exon 16 of cIL1RAP. In some embodiments, the CRISPR gene-editing system for the treatment of IDD comprising one or more sgRNAs targeting an exon of cIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eIL1RAP. In some embodiments, the CRISPR gene-editing system for the treatment of IDD comprises one or more sgRNAs targeting exon 2 of eIL1RAP. In some embodiments, the CRISPR gene-editing system for the treatment of IDD comprises one or more sgRNAs targeting exon 3 of eIL1RAP. In some embodiments, the CRISPR gene-editing system for the treatment of IDD comprises one or more sgRNAs targeting exon 4 of eIL1RAP. In some embodiments, the CRISPR gene-editing system for the treatment of IDD comprises one or more sgRNAs targeting exon 5 of eIL1RAP. In some embodiments, the CRISPR gene-editing system for the treatment of IDD comprises one or more sgRNAs targeting exon 6 of eIL1RAP. In some embodiments, the CRISPR gene-editing system for the treatment of IDD comprises one or more sgRNAs targeting exon 7 of eIL1RAP. In some embodiments, the CRISPR gene-editing system for the treatment of IDD comprises one or more sgRNAs targeting exon 8 of eIL1RAP. In some embodiments, the CRISPR gene-editing system for the treatment of IDD comprises one or more sgRNAs targeting exon 9 of eIL1RAP. In some embodiments, the CRISPR gene-editing system for the treatment of IDD comprises one or more sgRNAs targeting exon 10 of eIL1RAP. In some embodiments, the CRISPR gene-editing system for the treatment of IDD comprises one or more sgRNAs targeting exon 11 of eIL1RAP. In some embodiments, the CRISPR gene-editing system for the treatment of IDD comprises one or more sgRNAs targeting exon 12 of eIL1RAP. In some embodiments, the CRISPR gene-editing system for the treatment of IDD comprises one or more sgRNAs targeting exon 13 of eIL1RAP. In some embodiments, the CRISPR gene-editing system for the treatment of IDD comprising one or more sgRNAs targeting an exon of eIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fIL1RAP. In some embodiments, the CRISPR gene-editing system for the treatment of IDD comprises one or more sgRNAs targeting exon 2 of fIL1RAP. In some embodiments, the CRISPR gene-editing system for the treatment of IDD comprises one or more sgRNAs targeting exon 3 of fIL1RAP. In some embodiments, the CRISPR gene-editing system for the treatment of IDD comprises one or more sgRNAs targeting exon 4 of fIL1RAP. In some embodiments, the CRISPR gene-editing system for the treatment of IDD comprises one or more sgRNAs targeting exon 5 of fIL1RAP. In some embodiments, the CRISPR gene-editing system for the treatment of IDD comprises one or more sgRNAs targeting exon 6 of fIL1RAP. In some embodiments, the CRISPR gene-editing system for the treatment of IDD comprises one or more sgRNAs targeting exon 7 of fIL1RAP. In some embodiments, the CRISPR gene-editing system for the treatment of IDD comprises one or more sgRNAs targeting exon 8 of fIL1RAP. In some embodiments, the CRISPR gene-editing system for the treatment of IDD comprises one or more sgRNAs targeting exon 9 of fIL1RAP. In some embodiments, the CRISPR gene-editing system for the treatment of IDD comprises one or more sgRNAs targeting exon 10 of fIL1RAP. In some embodiments, the CRISPR gene-editing system for the treatment of IDD comprises one or more sgRNAs targeting exon 11 of fIL1RAP. In some embodiments, the CRISPR gene-editing system for the treatment of IDD comprises one or more sgRNAs targeting exon 12 of fIL1RAP. In some embodiments, the CRISPR gene-editing system for the treatment of IDD comprises one or more sgRNAs targeting exon 13 of fIL1RAP. In some embodiments, the CRISPR gene-editing system for the treatment of IDD comprises one or more sgRNAs targeting exon 14 of fIL1RAP. In some embodiments, the CRISPR gene-editing system for the treatment of IDD comprising one or more sgRNAs targeting an exon of fIL1RAP is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TGFBR1. In some embodiments, the CRISPR gene-editing system for the treatment of IDD comprises one or more sgRNAs targeting hTGFBR1. In some embodiments, the CRISPR gene-editing system for the treatment of IDD comprises one or more sgRNAs targeting cTGFBR1. In some embodiments, the CRISPR gene-editing system for the treatment of IDD comprises one or more sgRNAs targeting e TGFBR1. In some embodiments, the CRISPR gene-editing system for the treatment of IDD comprises one or more sgRNAs targeting fTGFBR1. In some embodiments, the CRISPR gene-editing system for the treatment of IDD comprising one or more sgRNAs targeting an exon of TGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTGFBR1. In some embodiments, the CRISPR gene-editing system for the treatment of IDD comprises one or more sgRNAs targeting exon 2 of hTGFBR1. In some embodiments, the CRISPR gene-editing system for the treatment of IDD comprises one or more sgRNAs targeting exon 3 of hTGFBR1. In some embodiments, the CRISPR gene-editing system for the treatment of IDD comprises one or more sgRNAs targeting exon 4 of hTGFBR1. In some embodiments, the CRISPR gene-editing system for the treatment of IDD comprises one or more sgRNAs targeting exon 5 of hTGFBR1. In some embodiments, the CRISPR gene-editing system for the treatment of IDD comprises one or more sgRNAs targeting exon 6 of hTGFBR1. In some embodiments, the CRISPR gene-editing system for the treatment of IDD comprises one or more sgRNAs targeting exon 7 of hTGFBR1. In some embodiments, the CRISPR gene-editing system for the treatment of IDD comprises one or more sgRNAs targeting exon 8 of hTGFBR1. In some embodiments, the CRISPR gene-editing system for the treatment of IDD comprises one or more sgRNAs targeting exon 9 of hTGFBR1. In some embodiments, the CRISPR gene-editing system for the treatment of IDD comprises one or more sgRNAs targeting exon 10 of hTGFBR1. In some embodiments, the CRISPR gene-editing system for the treatment of IDD comprises one or more sgRNAs targeting exon 11 of hTGFBR1. In some embodiments, the CRISPR gene-editing system for the treatment of IDD comprising one or more sgRNAs targeting an exon of hTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTGFBR1. In some embodiments, the CRISPR gene-editing system for the treatment of IDD comprises one or more sgRNAs targeting exon 2 of cTGFBR1. In some embodiments, the CRISPR gene-editing system for the treatment of IDD comprises one or more sgRNAs targeting exon 3 of cTGFBR1. In some embodiments, the CRISPR gene-editing system for the treatment of IDD comprises one or more sgRNAs targeting exon 4 of cTGFBR1. In some embodiments, the CRISPR gene-editing system for the treatment of IDD comprises one or more sgRNAs targeting exon 5 of cTGFBR1. In some embodiments, the CRISPR gene-editing system for the treatment of IDD comprises one or more sgRNAs targeting exon 6 of cTGFBR1. In some embodiments, the CRISPR gene-editing system for the treatment of IDD comprises one or more sgRNAs targeting exon 7 of cTGFBR1. In some embodiments, the CRISPR gene-editing system for the treatment of IDD comprises one or more sgRNAs targeting exon 8 of cTGFBR1. In some embodiments, the CRISPR gene-editing system for the treatment of IDD comprises one or more sgRNAs targeting exon 9 of cTGFBR1. In some embodiments, the CRISPR gene-editing system for the treatment of IDD comprising one or more sgRNAs targeting an exon of cTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTGFBR1. In some embodiments, the CRISPR gene-editing system for the treatment of IDD comprises one or more sgRNAs targeting exon 2 of eTGFBR1. In some embodiments, the CRISPR gene-editing system for the treatment of IDD comprises one or more sgRNAs targeting exon 3 of eTGFBR1. In some embodiments, the CRISPR gene-editing system for the treatment of IDD comprises one or more sgRNAs targeting exon 4 of eTGFBR1. In some embodiments, the CRISPR gene-editing system for the treatment of IDD comprises one or more sgRNAs targeting exon 5 of eTGFBR1. In some embodiments, the CRISPR gene-editing system for the treatment of IDD comprises one or more sgRNAs targeting exon 6 of eTGFBR1. In some embodiments, the CRISPR gene-editing system for the treatment of IDD comprises one or more sgRNAs targeting exon 7 of eTGFBR1. In some embodiments, the CRISPR gene-editing system for the treatment of IDD comprises one or more sgRNAs targeting exon 8 of eTGFBR1. In some embodiments, the CRISPR gene-editing system for the treatment of IDD comprises one or more sgRNAs targeting exon 9 of eTGFBR1. In some embodiments, the CRISPR gene-editing system for the treatment of IDD comprises one or more sgRNAs targeting exon 10 of eTGFBR1. In some embodiments, the CRISPR gene-editing system for the treatment of IDD comprises one or more sgRNAs targeting exon 11 of eTGFBR1. In some embodiments, the CRISPR gene-editing system for the treatment of IDD comprises one or more sgRNAs targeting exon 12 of eTGFBR1. In some embodiments, the CRISPR gene-editing system for the treatment of IDD comprising one or more sgRNAs targeting an exon of eTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTGFBR1. In some embodiments, the CRISPR gene-editing system for the treatment of IDD comprises one or more sgRNAs targeting exon 2 of fTGFBR1. In some embodiments, the CRISPR gene-editing system for the treatment of IDD comprises one or more sgRNAs targeting exon 3 of fTGFBR1. In some embodiments, the CRISPR gene-editing system for the treatment of IDD comprises one or more sgRNAs targeting exon 4 of fTGFBR1. In some embodiments, the CRISPR gene-editing system for the treatment of IDD comprises one or more sgRNAs targeting exon 5 of fTGFBR1. In some embodiments, the CRISPR gene-editing system for the treatment of IDD comprises one or more sgRNAs targeting exon 6 of fTGFBR1. In some embodiments, the CRISPR gene-editing system for the treatment of IDD comprises one or more sgRNAs targeting exon 7 of fTGFBR1. In some embodiments, the CRISPR gene-editing system for the treatment of IDD comprises one or more sgRNAs targeting exon 8 of fTGFBR1. In some embodiments, the CRISPR gene-editing system for the treatment of IDD comprises one or more sgRNAs targeting exon 9 of fTGFBR1. In some embodiments, the CRISPR gene-editing system for the treatment of IDD comprises one or more sgRNAs targeting exon 10 of fTGFBR1. In some embodiments, the CRISPR gene-editing system for the treatment of IDD comprises one or more sgRNAs targeting exon 11 of fTGFBR1. In some embodiments, the CRISPR gene-editing system for the treatment of IDD comprising one or more sgRNAs targeting an exon of fTGFBR1 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TGFBR2. In some embodiments, the CRISPR gene-editing system for the treatment of IDD comprises one or more sgRNAs targeting hTGFBR2. In some embodiments, the CRISPR gene-editing system for the treatment of IDD comprises one or more sgRNAs targeting cTGFBR2. In some embodiments, the CRISPR gene-editing system for the treatment of IDD comprises one or more sgRNAs targeting eTGFBR2. In some embodiments, the CRISPR gene-editing system for the treatment of IDD comprises one or more sgRNAs targeting fTGFBR2. In some embodiments, the CRISPR gene-editing system for the treatment of IDD comprising one or more sgRNAs targeting an exon of TGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTGFBR2. In some embodiments, the CRISPR gene-editing system for the treatment of IDD comprises one or more sgRNAs targeting exon 2 of hTGFBR2. In some embodiments, the CRISPR gene-editing system for the treatment of IDD comprises one or more sgRNAs targeting exon 3 of hTGFBR2. In some embodiments, the CRISPR gene-editing system for the treatment of IDD comprises one or more sgRNAs targeting exon 4 of hTGFBR2. In some embodiments, the CRISPR gene-editing system for the treatment of IDD comprises one or more sgRNAs targeting exon 5 of hTGFBR2. In some embodiments, the CRISPR gene-editing system for the treatment of IDD comprises one or more sgRNAs targeting exon 6 of hTGFBR2. In some embodiments, the CRISPR gene-editing system for the treatment of IDD comprises one or more sgRNAs targeting exon 7 of hTGFBR2. In some embodiments, the CRISPR gene-editing system for the treatment of IDD comprises one or more sgRNAs targeting exon 8 of hTGFBR2. In some embodiments, the CRISPR gene-editing system for the treatment of IDD comprises one or more sgRNAs targeting exon 9 of hTGFBR2. In some embodiments, the CRISPR gene-editing system for the treatment of IDD comprising one or more sgRNAs targeting an exon of hTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTGFBR2. In some embodiments, the CRISPR gene-editing system for the treatment of IDD comprises one or more sgRNAs targeting exon 2 of cTGFBR2. In some embodiments, the CRISPR gene-editing system for the treatment of IDD comprises one or more sgRNAs targeting exon 3 of cTGFBR2. In some embodiments, the CRISPR gene-editing system for the treatment of IDD comprises one or more sgRNAs targeting exon 4 of cTGFBR2. In some embodiments, the CRISPR gene-editing system for the treatment of IDD comprises one or more sgRNAs targeting exon 5 of cTGFBR2. In some embodiments, the CRISPR gene-editing system for the treatment of IDD comprises one or more sgRNAs targeting exon 6 of cTGFBR2. In some embodiments, the CRISPR gene-editing system for the treatment of IDD comprises one or more sgRNAs targeting exon 7 of cTGFBR2. In some embodiments, the CRISPR gene-editing system for the treatment of IDD comprises one or more sgRNAs targeting exon 8 of cTGFBR2. In some embodiments, the CRISPR gene-editing system for the treatment of IDD comprising one or more sgRNAs targeting an exon of cTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTGFBR2. In some embodiments, the CRISPR gene-editing system for the treatment of IDD comprises one or more sgRNAs targeting exon 2 of eTGFBR2. In some embodiments, the CRISPR gene-editing system for the treatment of IDD comprises one or more sgRNAs targeting exon 3 of eTGFBR2. In some embodiments, the CRISPR gene-editing system for the treatment of IDD comprises one or more sgRNAs targeting exon 4 of eTGFBR2. In some embodiments, the CRISPR gene-editing system for the treatment of IDD comprises one or more sgRNAs targeting exon 5 of eTGFBR2. In some embodiments, the CRISPR gene-editing system for the treatment of IDD comprises one or more sgRNAs targeting exon 6 of eTGFBR2. In some embodiments, the CRISPR gene-editing system for the treatment of IDD comprises one or more sgRNAs targeting exon 7 of eTGFBR2. In some embodiments, the CRISPR gene-editing system for the treatment of IDD comprises one or more sgRNAs targeting exon 8 of eTGFBR2. In some embodiments, the CRISPR gene-editing system for the treatment of IDD comprises one or more sgRNAs targeting exon 9 of eTGFBR2. In some embodiments, the CRISPR gene-editing system for the treatment of IDD comprises one or more sgRNAs targeting exon 10 of eTGFBR2. In some embodiments, the CRISPR gene-editing system for the treatment of IDD comprising one or more sgRNAs targeting an exon of eTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTGFBR2. In some embodiments, the CRISPR gene-editing system for the treatment of IDD comprises one or more sgRNAs targeting exon 2 of fTGFBR2. In some embodiments, the CRISPR gene-editing system for the treatment of IDD comprises one or more sgRNAs targeting exon 3 of fTGFBR2. In some embodiments, the CRISPR gene-editing system for the treatment of IDD comprises one or more sgRNAs targeting exon 4 of fTGFBR2. In some embodiments, the CRISPR gene-editing system for the treatment of IDD comprises one or more sgRNAs targeting exon 5 of fTGFBR2. In some embodiments, the CRISPR gene-editing system for the treatment of IDD comprises one or more sgRNAs targeting exon 6 of fTGFBR2. In some embodiments, the CRISPR gene-editing system for the treatment of IDD comprises one or more sgRNAs targeting exon 7 of fTGFBR2. In some embodiments, the CRISPR gene-editing system for the treatment of IDD comprises one or more sgRNAs targeting exon 8 of fTGFBR2. In some embodiments, the CRISPR gene-editing system for the treatment of IDD comprising one or more sgRNAs targeting an exon of fTGFBR2 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting IL6R. In some embodiments, the CRISPR gene-editing system for the treatment of IDD comprises one or more sgRNAs targeting hIL6R. In some embodiments, the CRISPR gene-editing system for the treatment of IDD comprises one or more sgRNAs targeting cIL6R. In some embodiments, the CRISPR gene-editing system for the treatment of IDD comprises one or more sgRNAs targeting eIL6R. In some embodiments, the CRISPR gene-editing system for the treatment of IDD comprises one or more sgRNAs targeting fIL6R. In some embodiments, the CRISPR gene-editing system for the treatment of IDD comprising one or more sgRNAs targeting an exon of IL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hIL6R. In some embodiments, the CRISPR gene-editing system for the treatment of IDD comprises one or more sgRNAs targeting exon 2 of hIL6R. In some embodiments, the CRISPR gene-editing system for the treatment of IDD comprises one or more sgRNAs targeting exon 3 of hIL6R. In some embodiments, the CRISPR gene-editing system for the treatment of IDD comprises one or more sgRNAs targeting exon 4 of hIL6R. In some embodiments, the CRISPR gene-editing system for the treatment of IDD comprises one or more sgRNAs targeting exon 5 of hIL6R. In some embodiments, the CRISPR gene-editing system for the treatment of IDD comprises one or more sgRNAs targeting exon 6 of hIL6R. In some embodiments, the CRISPR gene-editing system for the treatment of IDD comprises one or more sgRNAs targeting exon 7 of hIL6R. In some embodiments, the CRISPR gene-editing system for the treatment of IDD comprises one or more sgRNAs targeting exon 8 of hIL6R. In some embodiments, the CRISPR gene-editing system for the treatment of IDD comprises one or more sgRNAs targeting exon 9 of hIL6R. In some embodiments, the CRISPR gene-editing system for the treatment of IDD comprises one or more sgRNAs targeting exon 10 of hIL6R. In some embodiments, the CRISPR gene-editing system for the treatment of IDD comprises one or more sgRNAs targeting exon 11 of hIL6R In some embodiments, the CRISPR gene-editing system for the treatment of IDD comprises one or more sgRNAs targeting exon 12 of hIL6R. In some embodiments, the CRISPR gene-editing system for the treatment of IDD comprises one or more sgRNAs targeting exon 13 of hIL6R. In some embodiments, the CRISPR gene-editing system for the treatment of IDD comprises one or more sgRNAs targeting exon 14 of hIL6R. In some embodiments, the CRISPR gene-editing system for the treatment of IDD comprises one or more sgRNAs targeting exon 15 of hIL6R. In some embodiments, the CRISPR gene-editing system for the treatment of IDD comprising one or more sgRNAs targeting an exon of hIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cIL6R. In some embodiments, the CRISPR gene-editing system for the treatment of IDD comprises one or more sgRNAs targeting exon 2 of cIL6R. In some embodiments, the CRISPR gene-editing system for the treatment of IDD comprises one or more sgRNAs targeting exon 3 of cIL6R. In some embodiments, the CRISPR gene-editing system for the treatment of IDD comprises one or more sgRNAs targeting exon 4 of cIL6R. In some embodiments, the CRISPR gene-editing system for the treatment of IDD comprises one or more sgRNAs targeting exon 5 of cIL6R. In some embodiments, the CRISPR gene-editing system for the treatment of IDD comprises one or more sgRNAs targeting exon 6 of cIL6R. In some embodiments, the CRISPR gene-editing system for the treatment of IDD comprises one or more sgRNAs targeting exon 7 of cIL6R. In some embodiments, the CRISPR gene-editing system for the treatment of IDD comprises one or more sgRNAs targeting exon 8 of cIL6R. In some embodiments, the CRISPR gene-editing system for the treatment of IDD comprises one or more sgRNAs targeting exon 9 of cIL6R. In some embodiments, the CRISPR gene-editing system for the treatment of IDD comprises one or more sgRNAs targeting exon 10 of cIL6R. In some embodiments, the CRISPR gene-editing system for the treatment of IDD comprises one or more sgRNAs targeting exon 11 of cIL6R. In some embodiments, the CRISPR gene-editing system for the treatment of IDD comprises one or more sgRNAs targeting exon 12 of cIL6R. In some embodiments, the CRISPR gene-editing system for the treatment of IDD comprises one or more sgRNAs targeting exon 13 of cIL6R. In some embodiments, the CRISPR gene-editing system for the treatment of IDD comprising one or more sgRNAs targeting an exon of cIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eIL6R. In some embodiments, the CRISPR gene-editing system for the treatment of IDD comprises one or more sgRNAs targeting exon 2 of eIL6R. In some embodiments, the CRISPR gene-editing system for the treatment of IDD comprises one or more sgRNAs targeting exon 3 of eIL6R. In some embodiments, the CRISPR gene-editing system for the treatment of IDD comprises one or more sgRNAs targeting exon 4 of eIL6R. In some embodiments, the CRISPR gene-editing system for the treatment of IDD comprises one or more sgRNAs targeting exon 5 of eIL6R. In some embodiments, the CRISPR gene-editing system for the treatment of IDD comprises one or more sgRNAs targeting exon 6 of eIL6R. In some embodiments, the CRISPR gene-editing system for the treatment of IDD comprises one or more sgRNAs targeting exon 7 of eIL6R. In some embodiments, the CRISPR gene-editing system for the treatment of IDD comprises one or more sgRNAs targeting exon 8 of eIL6R. In some embodiments, the CRISPR gene-editing system for the treatment of IDD comprises one or more sgRNAs targeting exon 9 of eIL6R. In some embodiments, the CRISPR gene-editing system for the treatment of IDD comprises one or more sgRNAs targeting exon 10 of eIL6R. In some embodiments, the CRISPR gene-editing system for the treatment of IDD comprises one or more sgRNAs targeting exon 11 of eIL6R. In some embodiments, the CRISPR gene-editing system for the treatment of IDD comprising one or more sgRNAs targeting an exon of eIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fIL6R. In some embodiments, the CRISPR gene-editing system for the treatment of IDD comprises one or more sgRNAs targeting exon 2 of fIL6R. In some embodiments, the CRISPR gene-editing system for the treatment of IDD comprises one or more sgRNAs targeting exon 3 of fIL6R. In some embodiments, the CRISPR gene-editing system for the treatment of IDD comprises one or more sgRNAs targeting exon 4 of fIL6R. In some embodiments, the CRISPR gene-editing system for the treatment of IDD comprises one or more sgRNAs targeting exon 5 of fIL6R. In some embodiments, the CRISPR gene-editing system for the treatment of IDD comprises one or more sgRNAs targeting exon 6 of fIL6R. In some embodiments, the CRISPR gene-editing system for the treatment of IDD comprises one or more sgRNAs targeting exon 7 of fIL6R. In some embodiments, the CRISPR gene-editing system for the treatment of IDD comprises one or more sgRNAs targeting exon 8 of fIL6R. In some embodiments, the CRISPR gene-editing system for the treatment of IDD comprises one or more sgRNAs targeting exon 9 of fIL6R. In some embodiments, the CRISPR gene-editing system for the treatment of IDD comprises one or more sgRNAs targeting exon 10 of fIL6R. In some embodiments, the CRISPR gene-editing system for the treatment of IDD comprising one or more sgRNAs targeting an exon of fIL6R is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting IL6ST. In some embodiments, the CRISPR gene-editing system for the treatment of IDD comprises one or more sgRNAs targeting hIL6ST. In some embodiments, the CRISPR gene-editing system for the treatment of IDD comprises one or more sgRNAs targeting cIL6ST. In some embodiments, the CRISPR gene-editing system for the treatment of IDD comprises one or more sgRNAs targeting eIL6ST. In some embodiments, the CRISPR gene-editing system for the treatment of IDD comprises one or more sgRNAs targeting fIL6ST. In some embodiments, the CRISPR gene-editing system for the treatment of IDD comprising one or more sgRNAs targeting an exon of IL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hIL6ST. In some embodiments, the CRISPR gene-editing system for the treatment of IDD comprises one or more sgRNAs targeting exon 2 of hIL6ST. In some embodiments, the CRISPR gene-editing system for the treatment of IDD comprises one or more sgRNAs targeting exon 3 of hIL6ST. In some embodiments, the CRISPR gene-editing system for the treatment of IDD comprises one or more sgRNAs targeting exon 4 of hIL6ST. In some embodiments, the CRISPR gene-editing system for the treatment of IDD comprises one or more sgRNAs targeting exon 5 of hIL6ST. In some embodiments, the CRISPR gene-editing system for the treatment of IDD comprises one or more sgRNAs targeting exon 6 of hIL6ST. In some embodiments, the CRISPR gene-editing system for the treatment of IDD comprises one or more sgRNAs targeting exon 7 of hIL6ST. In some embodiments, the CRISPR gene-editing system for the treatment of IDD comprises one or more sgRNAs targeting exon 8 of hIL6ST. In some embodiments, the CRISPR gene-editing system for the treatment of IDD comprises one or more sgRNAs targeting exon 9 of hIL6ST. In some embodiments, the CRISPR gene-editing system for the treatment of IDD comprises one or more sgRNAs targeting exon 10 of hIL6ST. In some embodiments, the CRISPR gene-editing system for the treatment of IDD comprises one or more sgRNAs targeting exon 11 of hIL6ST. In some embodiments, the CRISPR gene-editing system for the treatment of IDD comprises one or more sgRNAs targeting exon 12 of hIL6ST. In some embodiments, the CRISPR gene-editing system for the treatment of IDD comprises one or more sgRNAs targeting exon 13 of hIL6ST. In some embodiments, the CRISPR gene-editing system for the treatment of IDD comprises one or more sgRNAs targeting exon 14 of hIL6ST. In some embodiments, the CRISPR gene-editing system for the treatment of IDD comprises one or more sgRNAs targeting exon 15 of hIL6ST. In some embodiments, the CRISPR gene-editing system for the treatment of IDD comprises one or more sgRNAs targeting exon 16 of hIL6ST. In some embodiments, the CRISPR gene-editing system for the treatment of IDD comprises one or more sgRNAs targeting exon 17 of hIL6ST. In some embodiments, the CRISPR gene-editing system for the treatment of IDD comprises one or more sgRNAs targeting exon 18 of hIL6ST. In some embodiments, the CRISPR gene-editing system for the treatment of IDD comprises one or more sgRNAs targeting exon 19 of hIL6ST. In some embodiments, the CRISPR gene-editing system for the treatment of IDD comprising one or more sgRNAs targeting an exon of hIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cIL6ST. In some embodiments, the CRISPR gene-editing system for the treatment of IDD comprises one or more sgRNAs targeting exon 2 of cIL6ST. In some embodiments, the CRISPR gene-editing system for the treatment of IDD comprises one or more sgRNAs targeting exon 3 of cIL6ST. In some embodiments, the CRISPR gene-editing system for the treatment of IDD comprises one or more sgRNAs targeting exon 4 of cIL6ST. In some embodiments, the CRISPR gene-editing system for the treatment of IDD comprises one or more sgRNAs targeting exon 5 of cIL6ST. In some embodiments, the CRISPR gene-editing system for the treatment of IDD comprises one or more sgRNAs targeting exon 6 of cIL6ST. In some embodiments, the CRISPR gene-editing system for the treatment of IDD comprises one or more sgRNAs targeting exon 7 of cIL6ST. In some embodiments, the CRISPR gene-editing system for the treatment of IDD comprises one or more sgRNAs targeting exon 8 of cIL6ST. In some embodiments, the CRISPR gene-editing system for the treatment of IDD comprises one or more sgRNAs targeting exon 9 of cIL6ST. In some embodiments, the CRISPR gene-editing system for the treatment of IDD comprises one or more sgRNAs targeting exon 10 of cIL6ST. In some embodiments, the CRISPR gene-editing system for the treatment of IDD comprises one or more sgRNAs targeting exon 11 of cIL6ST In some embodiments, the CRISPR gene-editing system for the treatment of IDD comprises one or more sgRNAs targeting exon 12 of cIL6ST. In some embodiments, the CRISPR gene-editing system for the treatment of IDD comprises one or more sgRNAs targeting exon 13 of cIL6ST. In some embodiments, the CRISPR gene-editing system for the treatment of IDD comprises one or more sgRNAs targeting exon 14 of cIL6ST. In some embodiments, the CRISPR gene-editing system for the treatment of IDD comprises one or more sgRNAs targeting exon 15 of cIL6ST. In some embodiments, the CRISPR gene-editing system for the treatment of IDD comprises one or more sgRNAs targeting exon 16 of cIL6ST. In some embodiments, the CRISPR gene-editing system for the treatment of IDD comprises one or more sgRNAs targeting exon 17 of cIL6ST. In some embodiments, the CRISPR gene-editing system for the treatment of IDD comprises one or more sgRNAs targeting exon 18 of cIL6ST. In some embodiments, the CRISPR gene-editing system for the treatment of IDD comprising one or more sgRNAs targeting an exon of cIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eIL6ST. In some embodiments, the CRISPR gene-editing system for the treatment of IDD comprises one or more sgRNAs targeting exon 2 of eIL6ST. In some embodiments, the CRISPR gene-editing system for the treatment of IDD comprises one or more sgRNAs targeting exon 3 of eIL6ST. In some embodiments, the CRISPR gene-editing system for the treatment of IDD comprises one or more sgRNAs targeting exon 4 of eIL6ST. In some embodiments, the CRISPR gene-editing system for the treatment of IDD comprises one or more sgRNAs targeting exon 5 of eIL6ST. In some embodiments, the CRISPR gene-editing system for the treatment of IDD comprises one or more sgRNAs targeting exon 6 of eIL6ST. In some embodiments, the CRISPR gene-editing system for the treatment of IDD comprises one or more sgRNAs targeting exon 7 of eIL6ST. In some embodiments, the CRISPR gene-editing system for the treatment of IDD comprises one or more sgRNAs targeting exon 8 of eIL6ST. In some embodiments, the CRISPR gene-editing system for the treatment of IDD comprises one or more sgRNAs targeting exon 9 of eIL6ST. In some embodiments, the CRISPR gene-editing system for the treatment of IDD comprises one or more sgRNAs targeting exon 10 of eIL6ST. In some embodiments, the CRISPR gene-editing system for the treatment of IDD comprises one or more sgRNAs targeting exon 11 of eIL6ST In some embodiments, the CRISPR gene-editing system for the treatment of IDD comprises one or more sgRNAs targeting exon 12 of eIL6ST. In some embodiments, the CRISPR gene-editing system for the treatment of IDD comprises one or more sgRNAs targeting exon 13 of eIL6ST. In some embodiments, the CRISPR gene-editing system for the treatment of IDD comprises one or more sgRNAs targeting exon 14 of eIL6ST. In some embodiments, the CRISPR gene-editing system for the treatment of IDD comprises one or more sgRNAs targeting exon 15 of eIL6ST. In some embodiments, the CRISPR gene-editing system for the treatment of IDD comprises one or more sgRNAs targeting exon 16 of eIL6ST. In some embodiments, the CRISPR gene-editing system for the treatment of IDD comprises one or more sgRNAs targeting exon 17 of eIL6ST. In some embodiments, the CRISPR gene-editing system for the treatment of IDD comprises one or more sgRNAs targeting exon 18 of eIL6ST. In some embodiments, the CRISPR gene-editing system for the treatment of IDD comprising one or more sgRNAs targeting an exon of eIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fIL6ST. In some embodiments, the CRISPR gene-editing system for the treatment of IDD comprises one or more sgRNAs targeting exon 2 of fIL6ST. In some embodiments, the CRISPR gene-editing system for the treatment of IDD comprises one or more sgRNAs targeting exon 3 of fIL6ST. In some embodiments, the CRISPR gene-editing system for the treatment of IDD comprises one or more sgRNAs targeting exon 4 of fIL6ST. In some embodiments, the CRISPR gene-editing system for the treatment of IDD comprises one or more sgRNAs targeting exon 5 of fIL6ST. In some embodiments, the CRISPR gene-editing system for the treatment of IDD comprises one or more sgRNAs targeting exon 6 of fIL6ST. In some embodiments, the CRISPR gene-editing system for the treatment of IDD comprises one or more sgRNAs targeting exon 7 of fIL6ST. In some embodiments, the CRISPR gene-editing system for the treatment of IDD comprises one or more sgRNAs targeting exon 8 of fIL6ST. In some embodiments, the CRISPR gene-editing system for the treatment of IDD comprises one or more sgRNAs targeting exon 9 of fIL6ST. In some embodiments, the CRISPR gene-editing system for the treatment of IDD comprises one or more sgRNAs targeting exon 10 of fIL6ST. In some embodiments, the CRISPR gene-editing system for the treatment of IDD comprises one or more sgRNAs targeting exon 11 of fIL6ST In some embodiments, the CRISPR gene-editing system for the treatment of IDD comprises one or more sgRNAs targeting exon 12 of fIL6ST. In some embodiments, the CRISPR gene-editing system for the treatment of IDD comprises one or more sgRNAs targeting exon 13 of fIL6ST. In some embodiments, the CRISPR gene-editing system for the treatment of IDD comprises one or more sgRNAs targeting exon 14 of fIL6ST. In some embodiments, the CRISPR gene-editing system for the treatment of IDD comprises one or more sgRNAs targeting exon 15 of fIL6ST. In some embodiments, the CRISPR gene-editing system for the treatment of IDD comprises one or more sgRNAs targeting exon 16 of fIL6ST. In some embodiments, the CRISPR gene-editing system for the treatment of IDD comprises one or more sgRNAs targeting exon 17 of fIL6ST. In some embodiments, the CRISPR gene-editing system for the treatment of IDD comprises one or more sgRNAs targeting exon 18 of fIL6ST. In some embodiments, the CRISPR gene-editing system for the treatment of IDD comprises one or more sgRNAs targeting exon 19 of fIL6ST. In some embodiments, the CRISPR gene-editing system for the treatment of IDD comprises one or more sgRNAs targeting exon 20 of fIL6ST. In some embodiments, the CRISPR gene-editing system for the treatment of IDD comprising one or more sgRNAs targeting an exon of fIL6ST is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TNFRSF1A. In some embodiments, the CRISPR gene-editing system for the treatment of IDD comprises one or more sgRNAs targeting hTNFRSF1A. In some embodiments, the CRISPR gene-editing system for the treatment of IDD comprises one or more sgRNAs targeting cTNFRSF1A. In some embodiments, the CRISPR gene-editing system for the treatment of IDD comprises one or more sgRNAs targeting eTNFRSF1A. In some embodiments, the CRISPR gene-editing system for the treatment of IDD comprises one or more sgRNAs targeting fTNFRSF1A. In some embodiments, the CRISPR gene-editing system for the treatment of IDD comprising one or more sgRNAs targeting an exon of TNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTNFRSF1A. In some embodiments, the CRISPR gene-editing system for the treatment of IDD comprises one or more sgRNAs targeting exon 2 of hTNFRSF1A. In some embodiments, the CRISPR gene-editing system for the treatment of IDD comprises one or more sgRNAs targeting exon 3 of hTNFRSF1A. In some embodiments, the CRISPR gene-editing system for the treatment of IDD comprises one or more sgRNAs targeting exon 4 of hTNFRSF1A. In some embodiments, the CRISPR gene-editing system for the treatment of IDD comprises one or more sgRNAs targeting exon 5 of hTNFRSF1A. In some embodiments, the CRISPR gene-editing system for the treatment of IDD comprises one or more sgRNAs targeting exon 6 of hTNFRSF1A. In some embodiments, the CRISPR gene-editing system for the treatment of IDD comprises one or more sgRNAs targeting exon 7 of hTNFRSF1A. In some embodiments, the CRISPR gene-editing system for the treatment of IDD comprises one or more sgRNAs targeting exon 8 of hTNFRSF1A. In some embodiments, the CRISPR gene-editing system for the treatment of IDD comprises one or more sgRNAs targeting exon 9 of hTNFRSF1A. In some embodiments, the CRISPR gene-editing system for the treatment of IDD comprises one or more sgRNAs targeting exon 10 of hTNFRSF1A. In some embodiments, the CRISPR gene-editing system for the treatment of IDD comprises one or more sgRNAs targeting exon 11 of hTNFRSF1A. In some embodiments, the CRISPR gene-editing system for the treatment of IDD comprising one or more sgRNAs targeting an exon of hTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTNFRSF1A. In some embodiments, the CRISPR gene-editing system for the treatment of IDD comprises one or more sgRNAs targeting exon 2 of cTNFRSF1A. In some embodiments, the CRISPR gene-editing system for the treatment of IDD comprises one or more sgRNAs targeting exon 3 of cTNFRSF1A. In some embodiments, the CRISPR gene-editing system for the treatment of IDD comprises one or more sgRNAs targeting exon 4 of cTNFRSF1A. In some embodiments, the CRISPR gene-editing system for the treatment of IDD comprises one or more sgRNAs targeting exon 5 of cTNFRSF1A. In some embodiments, the CRISPR gene-editing system for the treatment of IDD comprises one or more sgRNAs targeting exon 6 of cTNFRSF1A. In some embodiments, the CRISPR gene-editing system for the treatment of IDD comprises one or more sgRNAs targeting exon 7 of cTNFRSF1A. In some embodiments, the CRISPR gene-editing system for the treatment of IDD comprises one or more sgRNAs targeting exon 8 of cTNFRSF1A. In some embodiments, the CRISPR gene-editing system for the treatment of IDD comprises one or more sgRNAs targeting exon 9 of cTNFRSF1A. In some embodiments, the CRISPR gene-editing system for the treatment of IDD comprises one or more sgRNAs targeting exon 10 of cTNFRSF1A. In some embodiments, the CRISPR gene-editing system for the treatment of IDD comprises one or more sgRNAs targeting exon 11 of cTNFRSF1A. In some embodiments, the CRISPR gene-editing system for the treatment of IDD comprising one or more sgRNAs targeting an exon of cTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTNFRSF1A. In some embodiments, the CRISPR gene-editing system for the treatment of IDD comprises one or more sgRNAs targeting exon 2 of eTNFRSF1A. In some embodiments, the CRISPR gene-editing system for the treatment of IDD comprises one or more sgRNAs targeting exon 3 of eTNFRSF1A. In some embodiments, the CRISPR gene-editing system for the treatment of IDD comprises one or more sgRNAs targeting exon 4 of eTNFRSF1A. In some embodiments, the CRISPR gene-editing system for the treatment of IDD comprises one or more sgRNAs targeting exon 5 of eTNFRSF1A. In some embodiments, the CRISPR gene-editing system for the treatment of IDD comprises one or more sgRNAs targeting exon 6 of eTNFRSF1A. In some embodiments, the CRISPR gene-editing system for the treatment of IDD comprises one or more sgRNAs targeting exon 7 of eTNFRSF1A. In some embodiments, the CRISPR gene-editing system for the treatment of IDD comprises one or more sgRNAs targeting exon 8 of eTNFRSF1A. In some embodiments, the CRISPR gene-editing system for the treatment of IDD comprises one or more sgRNAs targeting exon 9 of eTNFRSF1A. In some embodiments, the CRISPR gene-editing system for the treatment of IDD comprises one or more sgRNAs targeting exon 10 of eTNFRSF1A. In some embodiments, the CRISPR gene-editing system for the treatment of IDD comprising one or more sgRNAs targeting an exon of eTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTNFRSF1A. In some embodiments, the CRISPR gene-editing system for the treatment of IDD comprises one or more sgRNAs targeting exon 2 of fTNFRSF1A. In some embodiments, the CRISPR gene-editing system for the treatment of IDD comprises one or more sgRNAs targeting exon 3 of fTNFRSF1A. In some embodiments, the CRISPR gene-editing system for the treatment of IDD comprises one or more sgRNAs targeting exon 4 of fTNFRSF1A. In some embodiments, the CRISPR gene-editing system for the treatment of IDD comprises one or more sgRNAs targeting exon 5 of fTNFRSF1A. In some embodiments, the CRISPR gene-editing system for the treatment of IDD comprises one or more sgRNAs targeting exon 6 of fTNFRSF1A. In some embodiments, the CRISPR gene-editing system for the treatment of IDD comprises one or more sgRNAs targeting exon 7 of fTNFRSF1A. In some embodiments, the CRISPR gene-editing system for the treatment of IDD comprises one or more sgRNAs targeting exon 8 of fTNFRSF1A. In some embodiments, the CRISPR gene-editing system for the treatment of IDD comprises one or more sgRNAs targeting exon 9 of fTNFRSF1A. In some embodiments, the CRISPR gene-editing system for the treatment of IDD comprises one or more sgRNAs targeting exon 10 of fTNFRSF1A. In some embodiments, the CRISPR gene-editing system for the treatment of IDD comprising one or more sgRNAs targeting an exon of fTNFRSF1A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TNFRSF1B. In some embodiments, the CRISPR gene-editing system for the treatment of IDD comprises one or more sgRNAs targeting hTNFRSF1B. In some embodiments, the CRISPR gene-editing system for the treatment of IDD comprises one or more sgRNAs targeting cTNFRSF1B. In some embodiments, the CRISPR gene-editing system for the treatment of IDD comprises one or more sgRNAs targeting eTNFRSF1B. In some embodiments, the CRISPR gene-editing system for the treatment of IDD comprises one or more sgRNAs targeting fTNFRSF1B. In some embodiments, the CRISPR gene-editing system for the treatment of IDD comprising one or more sgRNAs targeting an exon of TNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTNFRSF1B. In some embodiments, the CRISPR gene-editing system for the treatment of IDD comprises one or more sgRNAs targeting exon 2 of hTNFRSF1B. In some embodiments, the CRISPR gene-editing system for the treatment of IDD comprises one or more sgRNAs targeting exon 3 of hTNFRSF1B. In some embodiments, the CRISPR gene-editing system for the treatment of IDD comprises one or more sgRNAs targeting exon 4 of hTNFRSF1B. In some embodiments, the CRISPR gene-editing system for the treatment of IDD comprises one or more sgRNAs targeting exon 5 of hTNFRSF1B. In some embodiments, the CRISPR gene-editing system for the treatment of IDD comprises one or more sgRNAs targeting exon 6 of hTNFRSF1B. In some embodiments, the CRISPR gene-editing system for the treatment of IDD comprises one or more sgRNAs targeting exon 7 of hTNFRSF1B. In some embodiments, the CRISPR gene-editing system for the treatment of IDD comprises one or more sgRNAs targeting exon 8 of hTNFRSF1B. In some embodiments, the CRISPR gene-editing system for the treatment of IDD comprises one or more sgRNAs targeting exon 9 of hTNFRSF1B. In some embodiments, the CRISPR gene-editing system for the treatment of IDD comprises one or more sgRNAs targeting exon 10 of hTNFRSF1B. In some embodiments, the CRISPR gene-editing system for the treatment of IDD comprises one or more sgRNAs targeting exon 11 of hTNFRSF1B In some embodiments, the CRISPR gene-editing system for the treatment of IDD comprises one or more sgRNAs targeting exon 12 of hTNFRSF1B. In some embodiments, the CRISPR gene-editing system for the treatment of IDD comprises one or more sgRNAs targeting exon 13 of hTNFRSF1B. In some embodiments, the CRISPR gene-editing system for the treatment of IDD comprising one or more sgRNAs targeting an exon of hTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTNFRSF1B. In some embodiments, the CRISPR gene-editing system for the treatment of IDD comprises one or more sgRNAs targeting exon 2 of cTNFRSF1B. In some embodiments, the CRISPR gene-editing system for the treatment of IDD comprises one or more sgRNAs targeting exon 3 of cTNFRSF1B. In some embodiments, the CRISPR gene-editing system for the treatment of IDD comprises one or more sgRNAs targeting exon 4 of cTNFRSF1B. In some embodiments, the CRISPR gene-editing system for the treatment of IDD comprises one or more sgRNAs targeting exon 5 of cTNFRSF1B. In some embodiments, the CRISPR gene-editing system for the treatment of IDD comprises one or more sgRNAs targeting exon 6 of cTNFRSF1B. In some embodiments, the CRISPR gene-editing system for the treatment of IDD comprises one or more sgRNAs targeting exon 7 of cTNFRSF1B. In some embodiments, the CRISPR gene-editing system for the treatment of IDD comprises one or more sgRNAs targeting exon 8 of cTNFRSF1B. In some embodiments, the CRISPR gene-editing system for the treatment of IDD comprises one or more sgRNAs targeting exon 9 of cTNFRSF1B. In some embodiments, the CRISPR gene-editing system for the treatment of IDD comprises one or more sgRNAs targeting exon 10 of cTNFRSF1B. In some embodiments, the CRISPR gene-editing system for the treatment of IDD comprises one or more sgRNAs targeting exon 11 of cTNFRSF1B. In some embodiments, the CRISPR gene-editing system for the treatment of IDD comprising one or more sgRNAs targeting an exon of cTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTNFRSF1B. In some embodiments, the CRISPR gene-editing system for the treatment of IDD comprises one or more sgRNAs targeting exon 2 of eTNFRSF1B. In some embodiments, the CRISPR gene-editing system for the treatment of IDD comprises one or more sgRNAs targeting exon 3 of eTNFRSF1B. In some embodiments, the CRISPR gene-editing system for the treatment of IDD comprises one or more sgRNAs targeting exon 4 of eTNFRSF1B. In some embodiments, the CRISPR gene-editing system for the treatment of IDD comprises one or more sgRNAs targeting exon 5 of eTNFRSF1B. In some embodiments, the CRISPR gene-editing system for the treatment of IDD comprises one or more sgRNAs targeting exon 6 of eTNFRSF1B. In some embodiments, the CRISPR gene-editing system for the treatment of IDD comprises one or more sgRNAs targeting exon 7 of eTNFRSF1B. In some embodiments, the CRISPR gene-editing system for the treatment of IDD comprises one or more sgRNAs targeting exon 8 of eTNFRSF1B. In some embodiments, the CRISPR gene-editing system for the treatment of IDD comprises one or more sgRNAs targeting exon 9 of eTNFRSF1B. In some embodiments, the CRISPR gene-editing system for the treatment of IDD comprises one or more sgRNAs targeting exon 10 of eTNFRSF1B. In some embodiments, the CRISPR gene-editing system for the treatment of IDD comprises one or more sgRNAs targeting exon 11 of eTNFRSF1B. In some embodiments, the CRISPR gene-editing system for the treatment of IDD comprising one or more sgRNAs targeting an exon of eTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTNFRSF1B. In some embodiments, the CRISPR gene-editing system for the treatment of IDD comprises one or more sgRNAs targeting exon 2 of fTNFRSF1B. In some embodiments, the CRISPR gene-editing system for the treatment of IDD comprises one or more sgRNAs targeting exon 3 of fTNFRSF1B. In some embodiments, the CRISPR gene-editing system for the treatment of IDD comprises one or more sgRNAs targeting exon 4 of fTNFRSF1B. In some embodiments, the CRISPR gene-editing system for the treatment of IDD comprises one or more sgRNAs targeting exon 5 of fTNFRSF1B. In some embodiments, the CRISPR gene-editing system for the treatment of IDD comprises one or more sgRNAs targeting exon 6 of fTNFRSF1B. In some embodiments, the CRISPR gene-editing system for the treatment of IDD comprises one or more sgRNAs targeting exon 7 of fTNFRSF1B. In some embodiments, the CRISPR gene-editing system for the treatment of IDD comprises one or more sgRNAs targeting exon 8 of fTNFRSF1B. In some embodiments, the CRISPR gene-editing system for the treatment of IDD comprises one or more sgRNAs targeting exon 9 of fTNFRSF1B. In some embodiments, the CRISPR gene-editing system for the treatment of IDD comprises one or more sgRNAs targeting exon 10 of fTNFRSF1B. In some embodiments, the CRISPR gene-editing system for the treatment of IDD comprising one or more sgRNAs targeting an exon of fTNFRSF1B is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TNFRSF3. In some embodiments, the CRISPR gene-editing system for the treatment of IDD comprises one or more sgRNAs targeting hTNFRSF3. In some embodiments, the CRISPR gene-editing system for the treatment of IDD comprises one or more sgRNAs targeting cTNFRSF3. In some embodiments, the CRISPR gene-editing system for the treatment of IDD comprises one or more sgRNAs targeting eTNFRSF3. In some embodiments, the CRISPR gene-editing system for the treatment of IDD comprises one or more sgRNAs targeting fTNFRSF3. In some embodiments, the CRISPR gene-editing system for the treatment of IDD comprising one or more sgRNAs targeting an exon of TNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTNFRSF3. In some embodiments, the CRISPR gene-editing system for the treatment of IDD comprises one or more sgRNAs targeting exon 2 of hTNFRSF3. In some embodiments, the CRISPR gene-editing system for the treatment of IDD comprises one or more sgRNAs targeting exon 3 of hTNFRSF3. In some embodiments, the CRISPR gene-editing system for the treatment of IDD comprises one or more sgRNAs targeting exon 4 of hTNFRSF3. In some embodiments, the CRISPR gene-editing system for the treatment of IDD comprises one or more sgRNAs targeting exon 5 of hTNFRSF3. In some embodiments, the CRISPR gene-editing system for the treatment of IDD comprises one or more sgRNAs targeting exon 6 of hTNFRSF3. In some embodiments, the CRISPR gene-editing system for the treatment of IDD comprises one or more sgRNAs targeting exon 7 of hTNFRSF3. In some embodiments, the CRISPR gene-editing system for the treatment of IDD comprises one or more sgRNAs targeting exon 8 of hTNFRSF3. In some embodiments, the CRISPR gene-editing system for the treatment of IDD comprises one or more sgRNAs targeting exon 9 of hTNFRSF3. In some embodiments, the CRISPR gene-editing system for the treatment of IDD comprises one or more sgRNAs targeting exon 10 of hTNFRSF3. In some embodiments, the CRISPR gene-editing system for the treatment of IDD comprises one or more sgRNAs targeting exon 11 of hTNFRSF3. In some embodiments, the CRISPR gene-editing system for the treatment of IDD comprises one or more sgRNAs targeting exon 12 of hTNFRSF3. In some embodiments, the CRISPR gene-editing system for the treatment of IDD comprising one or more sgRNAs targeting an exon of hTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTNFRSF3. In some embodiments, the CRISPR gene-editing system for the treatment of IDD comprises one or more sgRNAs targeting exon 2 of cTNFRSF3. In some embodiments, the CRISPR gene-editing system for the treatment of IDD comprises one or more sgRNAs targeting exon 3 of cTNFRSF3. In some embodiments, the CRISPR gene-editing system for the treatment of IDD comprises one or more sgRNAs targeting exon 4 of cTNFRSF3. In some embodiments, the CRISPR gene-editing system for the treatment of IDD comprises one or more sgRNAs targeting exon 5 of cTNFRSF3. In some embodiments, the CRISPR gene-editing system for the treatment of IDD comprises one or more sgRNAs targeting exon 6 of cTNFRSF3. In some embodiments, the CRISPR gene-editing system for the treatment of IDD comprises one or more sgRNAs targeting exon 7 of cTNFRSF3. In some embodiments, the CRISPR gene-editing system for the treatment of IDD comprises one or more sgRNAs targeting exon 8 of cTNFRSF3. In some embodiments, the CRISPR gene-editing system for the treatment of IDD comprises one or more sgRNAs targeting exon 9 of cTNFRSF3. In some embodiments, the CRISPR gene-editing system for the treatment of IDD comprises one or more sgRNAs targeting exon 10 of cTNFRSF3. In some embodiments, the CRISPR gene-editing system for the treatment of IDD comprising one or more sgRNAs targeting an exon of cTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTNFRSF3. In some embodiments, the CRISPR gene-editing system for the treatment of IDD comprises one or more sgRNAs targeting exon 2 of eTNFRSF3. In some embodiments, the CRISPR gene-editing system for the treatment of IDD comprises one or more sgRNAs targeting exon 3 of eTNFRSF3. In some embodiments, the CRISPR gene-editing system for the treatment of IDD comprises one or more sgRNAs targeting exon 4 of eTNFRSF3. In some embodiments, the CRISPR gene-editing system for the treatment of IDD comprises one or more sgRNAs targeting exon 5 of eTNFRSF3. In some embodiments, the CRISPR gene-editing system for the treatment of IDD comprises one or more sgRNAs targeting exon 6 of eTNFRSF3. In some embodiments, the CRISPR gene-editing system for the treatment of IDD comprises one or more sgRNAs targeting exon 7 of eTNFRSF3. In some embodiments, the CRISPR gene-editing system for the treatment of IDD comprises one or more sgRNAs targeting exon 8 of eTNFRSF3. In some embodiments, the CRISPR gene-editing system for the treatment of IDD comprises one or more sgRNAs targeting exon 9 of eTNFRSF3. In some embodiments, the CRISPR gene-editing system for the treatment of IDD comprises one or more sgRNAs targeting exon 10 of eTNFRSF3. In some embodiments, the CRISPR gene-editing system for the treatment of IDD comprising one or more sgRNAs targeting an exon of eTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTNFRSF3. In some embodiments, the CRISPR gene-editing system for the treatment of IDD comprises one or more sgRNAs targeting exon 2 of fTNFRSF3. In some embodiments, the CRISPR gene-editing system for the treatment of IDD comprises one or more sgRNAs targeting exon 3 of fTNFRSF3. In some embodiments, the CRISPR gene-editing system for the treatment of IDD comprises one or more sgRNAs targeting exon 4 of fTNFRSF3. In some embodiments, the CRISPR gene-editing system for the treatment of IDD comprises one or more sgRNAs targeting exon 5 of fTNFRSF3. In some embodiments, the CRISPR gene-editing system for the treatment of IDD comprises one or more sgRNAs targeting exon 6 of fTNFRSF3. In some embodiments, the CRISPR gene-editing system for the treatment of IDD comprises one or more sgRNAs targeting exon 7 of fTNFRSF3. In some embodiments, the CRISPR gene-editing system for the treatment of IDD comprises one or more sgRNAs targeting exon 8 of fTNFRSF3. In some embodiments, the CRISPR gene-editing system for the treatment of IDD comprises one or more sgRNAs targeting exon 9 of fTNFRSF3. In some embodiments, the CRISPR gene-editing system for the treatment of IDD comprises one or more sgRNAs targeting exon 10 of fTNFRSF3. In some embodiments, the CRISPR gene-editing system for the treatment of IDD comprising one or more sgRNAs targeting an exon of fTNFRSF3 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TNFRSF4. In some embodiments, the CRISPR gene-editing system for the treatment of IDD comprises one or more sgRNAs targeting hTNFRSF4. In some embodiments, the CRISPR gene-editing system for the treatment of IDD comprises one or more sgRNAs targeting cTNFRSF4. In some embodiments, the CRISPR gene-editing system for the treatment of IDD comprises one or more sgRNAs targeting eTNFRSF4. In some embodiments, the CRISPR gene-editing system for the treatment of IDD comprises one or more sgRNAs targeting fTNFRSF4. In some embodiments, the CRISPR gene-editing system for the treatment of IDD comprising one or more sgRNAs targeting an exon of TNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTNFRSF4. In some embodiments, the CRISPR gene-editing system for the treatment of IDD comprises one or more sgRNAs targeting exon 2 of hTNFRSF4. In some embodiments, the CRISPR gene-editing system for the treatment of IDD comprises one or more sgRNAs targeting exon 3 of hTNFRSF4. In some embodiments, the CRISPR gene-editing system for the treatment of IDD comprises one or more sgRNAs targeting exon 4 of hTNFRSF4. In some embodiments, the CRISPR gene-editing system for the treatment of IDD comprises one or more sgRNAs targeting exon 5 of hTNFRSF4. In some embodiments, the CRISPR gene-editing system for the treatment of IDD comprises one or more sgRNAs targeting exon 6 of hTNFRSF4. In some embodiments, the CRISPR gene-editing system for the treatment of IDD comprising one or more sgRNAs targeting an exon of hTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTNFRSF4. In some embodiments, the CRISPR gene-editing system for the treatment of IDD comprises one or more sgRNAs targeting exon 2 of cTNFRSF4. In some embodiments, the CRISPR gene-editing system for the treatment of IDD comprises one or more sgRNAs targeting exon 3 of cTNFRSF4. In some embodiments, the CRISPR gene-editing system for the treatment of IDD comprises one or more sgRNAs targeting exon 4 of cTNFRSF4. In some embodiments, the CRISPR gene-editing system for the treatment of IDD comprises one or more sgRNAs targeting exon 5 of cTNFRSF4. In some embodiments, the CRISPR gene-editing system for the treatment of IDD comprises one or more sgRNAs targeting exon 6 of cTNFRSF4. In some embodiments, the CRISPR gene-editing system for the treatment of IDD comprises one or more sgRNAs targeting exon 7 of cTNFRSF4. In some embodiments, the CRISPR gene-editing system for the treatment of IDD comprising one or more sgRNAs targeting an exon of cTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTNFRSF4. In some embodiments, the CRISPR gene-editing system for the treatment of IDD comprises one or more sgRNAs targeting exon 2 of eTNFRSF4. In some embodiments, the CRISPR gene-editing system for the treatment of IDD comprises one or more sgRNAs targeting exon 3 of eTNFRSF4. In some embodiments, the CRISPR gene-editing system for the treatment of IDD comprises one or more sgRNAs targeting exon 4 of eTNFRSF4. In some embodiments, the CRISPR gene-editing system for the treatment of IDD comprises one or more sgRNAs targeting exon 5 of eTNFRSF4. In some embodiments, the CRISPR gene-editing system for the treatment of IDD comprises one or more sgRNAs targeting exon 6 of eTNFRSF4. In some embodiments, the CRISPR gene-editing system for the treatment of IDD comprises one or more sgRNAs targeting exon 7 of eTNFRSF4. In some embodiments, the CRISPR gene-editing system for the treatment of IDD comprising one or more sgRNAs targeting an exon of eTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTNFRSF4. In some embodiments, the CRISPR gene-editing system for the treatment of IDD comprises one or more sgRNAs targeting exon 2 of fTNFRSF4. In some embodiments, the CRISPR gene-editing system for the treatment of IDD comprises one or more sgRNAs targeting exon 3 of fTNFRSF4. In some embodiments, the CRISPR gene-editing system for the treatment of IDD comprises one or more sgRNAs targeting exon 4 of fTNFRSF4. In some embodiments, the CRISPR gene-editing system for the treatment of IDD comprises one or more sgRNAs targeting exon 5 of fTNFRSF4. In some embodiments, the CRISPR gene-editing system for the treatment of IDD comprises one or more sgRNAs targeting exon 6 of fTNFRSF4. In some embodiments, the CRISPR gene-editing system for the treatment of IDD comprises one or more sgRNAs targeting exon 7 of fTNFRSF4. In some embodiments, the CRISPR gene-editing system for the treatment of IDD comprising one or more sgRNAs targeting an exon of fTNFRSF4 is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting TNFRSF11A. In some embodiments, the CRISPR gene-editing system for the treatment of IDD comprises one or more sgRNAs targeting hTNFRSF11A. In some embodiments, the CRISPR gene-editing system for the treatment of IDD comprises one or more sgRNAs targeting cTNFRSF11A. In some embodiments, the CRISPR gene-editing system for the treatment of IDD comprises one or more sgRNAs targeting eTNFRSF11A. In some embodiments, the CRISPR gene-editing system for the treatment of IDD comprises one or more sgRNAs targeting fTNFRSF11A. In some embodiments, the CRISPR gene-editing system for the treatment of IDD comprising one or more sgRNAs targeting an exon of TNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of hTNFRSF11A. In some embodiments, the CRISPR gene-editing system for the treatment of IDD comprises one or more sgRNAs targeting exon 2 of hTNFRSF11A. In some embodiments, the CRISPR gene-editing system for the treatment of IDD comprises one or more sgRNAs targeting exon 3 of hTNFRSF11A. In some embodiments, the CRISPR gene-editing system for the treatment of IDD comprises one or more sgRNAs targeting exon 4 of hTNFRSF11A. In some embodiments, the CRISPR gene-editing system for the treatment of IDD comprises one or more sgRNAs targeting exon 5 of hTNFRSF11A. In some embodiments, the CRISPR gene-editing system for the treatment of IDD comprises one or more sgRNAs targeting exon 6 of hTNFRSF11A. In some embodiments, the CRISPR gene-editing system for the treatment of IDD comprises one or more sgRNAs targeting exon 7 of hTNFRSF11A. In some embodiments, the CRISPR gene-editing system for the treatment of IDD comprises one or more sgRNAs targeting exon 8 of hTNFRSF11A. In some embodiments, the CRISPR gene-editing system for the treatment of IDD comprises one or more sgRNAs targeting exon 9 of hTNFRSF11A. In some embodiments, the CRISPR gene-editing system for the treatment of IDD comprises one or more sgRNAs targeting exon 10 of hTNFRSF11A. In some embodiments, the CRISPR gene-editing system for the treatment of IDD comprises one or more sgRNAs targeting exon 11 of hTNFRSF11A. In some embodiments, the CRISPR gene-editing system for the treatment of IDD comprises one or more sgRNAs targeting exon 12 of hTNFRSF11A. In some embodiments, the CRISPR gene-editing system for the treatment of IDD comprising one or more sgRNAs targeting an exon of hTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of cTNFRSF11A. In some embodiments, the CRISPR gene-editing system for the treatment of IDD comprises one or more sgRNAs targeting exon 2 of cTNFRSF11A. In some embodiments, the CRISPR gene-editing system for the treatment of IDD comprises one or more sgRNAs targeting exon 3 of cTNFRSF11A. In some embodiments, the CRISPR gene-editing system for the treatment of IDD comprises one or more sgRNAs targeting exon 4 of cTNFRSF11A. In some embodiments, the CRISPR gene-editing system for the treatment of IDD comprises one or more sgRNAs targeting exon 5 of cTNFRSF11A. In some embodiments, the CRISPR gene-editing system for the treatment of IDD comprises one or more sgRNAs targeting exon 6 of cTNFRSF11A. In some embodiments, the CRISPR gene-editing system for the treatment of IDD comprises one or more sgRNAs targeting exon 7 of cTNFRSF11A. In some embodiments, the CRISPR gene-editing system for the treatment of IDD comprises one or more sgRNAs targeting exon 8 of cTNFRSF11A. In some embodiments, the CRISPR gene-editing system for the treatment of IDD comprises one or more sgRNAs targeting exon 9 of cTNFRSF11A. In some embodiments, the CRISPR gene-editing system for the treatment of IDD comprises one or more sgRNAs targeting exon 10 of cTNFRSF11A. In some embodiments, the CRISPR gene-editing system for the treatment of IDD comprises one or more sgRNAs targeting exon 11 of cTNFRSF11A. In some embodiments, the CRISPR gene-editing system for the treatment of IDD comprising one or more sgRNAs targeting an exon of cTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of eTNFRSF11A. In some embodiments, the CRISPR gene-editing system for the treatment of IDD comprises one or more sgRNAs targeting exon 2 of eTNFRSF11A. In some embodiments, the CRISPR gene-editing system for the treatment of IDD comprises one or more sgRNAs targeting exon 3 of eTNFRSF11A. In some embodiments, the CRISPR gene-editing system for the treatment of IDD comprises one or more sgRNAs targeting exon 4 of eTNFRSF11A. In some embodiments, the CRISPR gene-editing system for the treatment of IDD comprises one or more sgRNAs targeting exon 5 of eTNFRSF11A. In some embodiments, the CRISPR gene-editing system for the treatment of IDD comprises one or more sgRNAs targeting exon 6 of eTNFRSF11A. In some embodiments, the CRISPR gene-editing system for the treatment of IDD comprises one or more sgRNAs targeting exon 7 of eTNFRSF11A. In some embodiments, the CRISPR gene-editing system for the treatment of IDD comprises one or more sgRNAs targeting exon 8 of eTNFRSF11A. In some embodiments, the CRISPR gene-editing system for the treatment of IDD comprises one or more sgRNAs targeting exon 9 of eTNFRSF11A. In some embodiments, the CRISPR gene-editing system for the treatment of IDD comprises one or more sgRNAs targeting exon 10 of eTNFRSF11A. In some embodiments, the CRISPR gene-editing system for the treatment of IDD comprising one or more sgRNAs targeting an exon of eTNFRSF11A is delivered via one or more lipid nanoparticles (LNPs), the LNPs comprising an LNP system described herein, e.g., and without limitation, any one of LNP systems LNP001 to LNP240.

In some embodiments, the CRISPR gene-editing system for the treatment of IDD comprises one or more sgRNAs targeting exon 1 of fTNFRSF11A. In some embodiments, the CRISPR gene-editing system for the treatment of IDD comprises one or more sgRNAs targeting exon 2 of fTNFRSF11A. In some embodiments, the CRISPR gene-editing system for the treatment of IDD comprises one or more sgRNAs targeting exon 3 of fTNFRSF11A. In some embodiments, the CRISPR gene-editing system for the treatment of IDD comprises one or more sgRNAs targeting exon 4 of fTNFRSF11A. In some embodiments, the CRISPR gene-editing system for the treatment of IDD comprises one or more sgRNAs targeting exon 5 of fTNFRSF11A. In some embodiments, the CRISPR gene-editing system for the treatment of IDD comprises one or more sgRNAs targeting exon 6 of fTNFRSF11A. In some embodiments, the CRISPR gene-editing system for the treatment of IDD comprises one or more sgRNAs targeting exon 7 of fTNFRSF11A. In some embodiments, the CRISPR gene-editing system for the treatment of IDD comprises one or more sgRNAs targeting exon 8 of fTNFRSF11A. In some embodiments, the CRISPR gene-editing system for the treatment of IDD comprises one or more sgRNAs targeting exon 9 of fTNFRSF11A. In some embodiments, the CRISPR gene-editing system for the treatment of IDD comprises one or more sgRNAs targeting exon 10 of fTNFRSF11A. In some embodiments, the CRISPR gene-editing system for the treatment of IDD comprises one or more sgRNAs targeting exon 11 of fTNFRSF11A. In some embodiments, the CRISPR gene-editing system for the treatment of IDD comprising one or more sgRNAs targeting an exon of fTNFRSF11A is delivered via one or more lipid nanoparticles (LNPs), the LNPs comprising an LNP system described herein, e.g., and without limitation, any one of LNP systems LNP001 to LNP240.

### VI. Delivery

### A. Lipid Nanoparticles (LNP)

In some embodiments, a CRISPR gene-editing system is delivered by a nanoparticle. Without wishing to be bound by any particular theory, in certain embodiments, nucleic acids, when present in the nanoparticle, are resistant in aqueous solution to degradation with a nuclease. In other embodiments, proteins are protected from protease degradation. In some embodiments, proteins and nucleic acids encapsulated by nanoparticles are capable of penetrating the cellular plasma membrane.

Lipid nanoparticles comprising nucleic acids and their method of preparation is disclosed in at least WO2017/019935, WO2017/049074, WO2017/201346, WO2017/218704, WO2018/006052, WO2018/013525, WO2018/089540, WO2018/119115, WO2018/126084, WO2018/157009, WO2018/170336, WO2018/222890, WO2019/046809, WO2019/089828, WO2020/061284, WO2020/061317. WO2020/081938, WO2020/097511, WO2020/097520, WO2020/097540, WO2020/097548, WO2020/214946, WO2020/219941, WO2020/232276, WO2020/227615, WO2020/061295, WO2021/007278, WO2021/016430, WO2021/021988, EP Patent No. EP 2 972 360, US20200155691, US20200237671, U.S. Pat. Nos. 8,058,069, 8,492,359, 8,822,668, 9,364,435, 9,404,127, 9,504,651, 9,593,077, 9,738,593, 9,868,691, 9,868,692, 9,950,068, 10,138,213, 10,166,298, 10,221,127, 10,238,754, 10,266,485, 10,383,952, 10,730,924, 10,766,852, 11,079,379, 11,141,378 and 11,246,933, which are incorporated herein by reference in their entirety for all purposes.

### Lipid Nanoparticle Compositions

In some embodiments, the largest dimension of a nanoparticle composition is 1 micrometer or shorter (e.g., 1 micrometer, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method. Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers may be functionalized and/or crosslinked to one another. Lipid bilayers may include one or more ligands, proteins, or channels. In various embodiments, lipid nanoparticles described herein have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 nm to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm, and are substantially non-toxic.

In certain embodiments, the lipid nanoparticles described herein comprise one or more components, including a lipid component, and (optionally) a structural component. The lipid component comprises lipids selected from ionizable and/or cationic lipids (i.e., lipids that may have a positive or partial positive charge at physiological pH), neutral lipids (e.g., phospholipids, or sphingolipids), and polymer-conjugated lipids (e.g., PEGylated lipids). In some embodiments, the lipid component comprises a single ionizable lipid. In other embodiments, the lipid component comprises 2, 3, 4, 5, 6, 7, 8, 9, 10, 15, or 20 ionizable lipids. In some embodiments, the lipid component comprises a single neutral lipid. In other embodiments, the lipid component comprises 2, 3, 4, 5, 6, 7, 8, 9, 10, 15, or 20 neutral lipids. In some embodiments, the lipid component comprises a single polymer-conjugated lipid. In other embodiments, the lipid component comprises 2, 3, 4, 5, 6, 7, 8, 9, 10, 15, or 20 polymer-conjugated lipids. In some embodiments, the structural component comprises a single structural lipid. In other embodiments, the structural component comprises 2, 3, 4, 5, 6, 7, 8, 9, 10, 15, or 20 structural lipids. In some embodiments, the lipid component comprises at least one cationic lipid, at least one neutral lipid, and at least one polymer-conjugated lipid. The present disclosure contemplates that the lipid component may comprise any combination of the foregoing constituents.

### Ionizable/Cationic Lipids

In some embodiments, the lipid component comprises an ionizable lipid. In some embodiments, the ionizable lipid is anionic. In other embodiments, the ionizable lipid is a cationic lipid. In some embodiments, the lipid component comprises cationic lipids including, but not limited to, a cationic lipid selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and mixtures thereof.

Non-exhaustive and non-limiting examples of cationic lipids include:

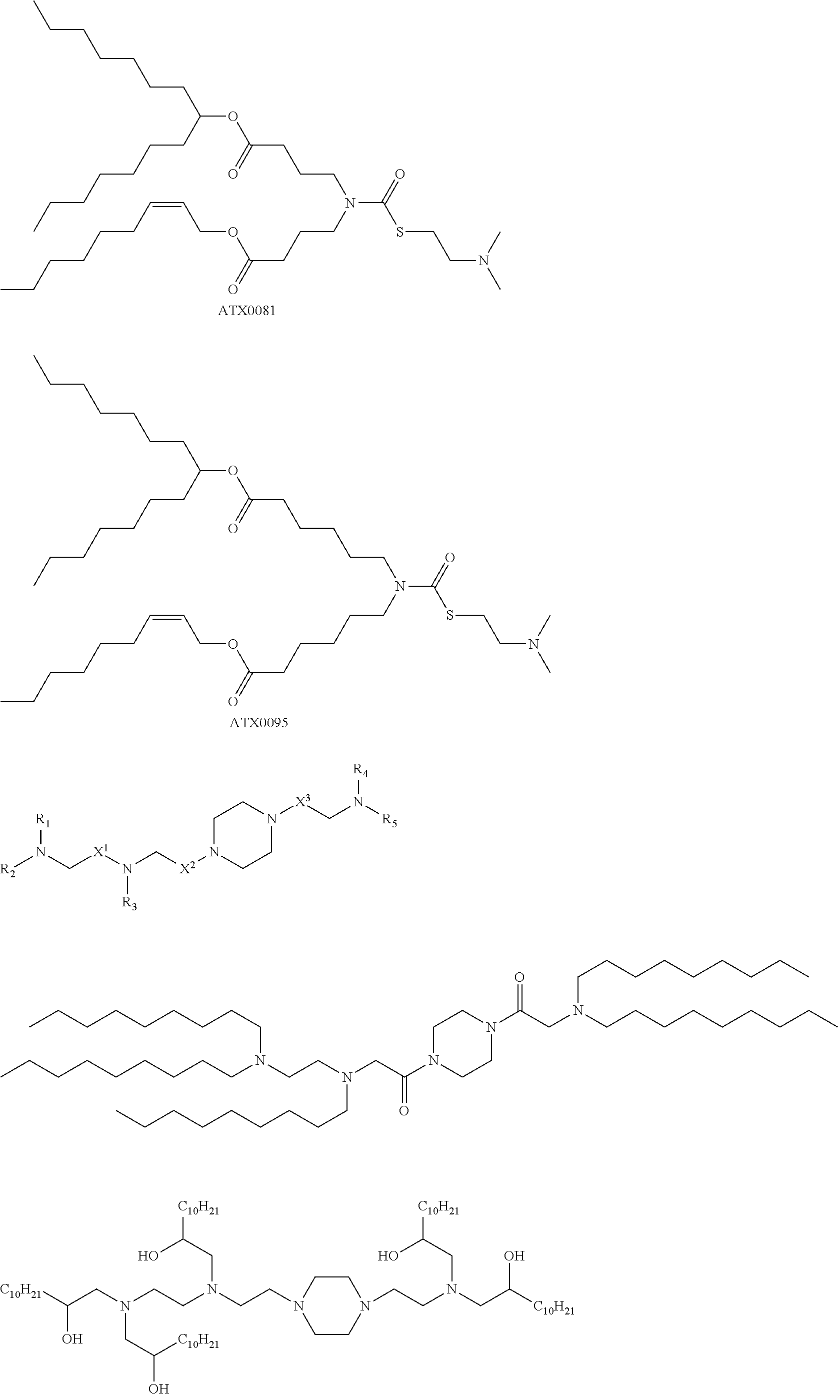

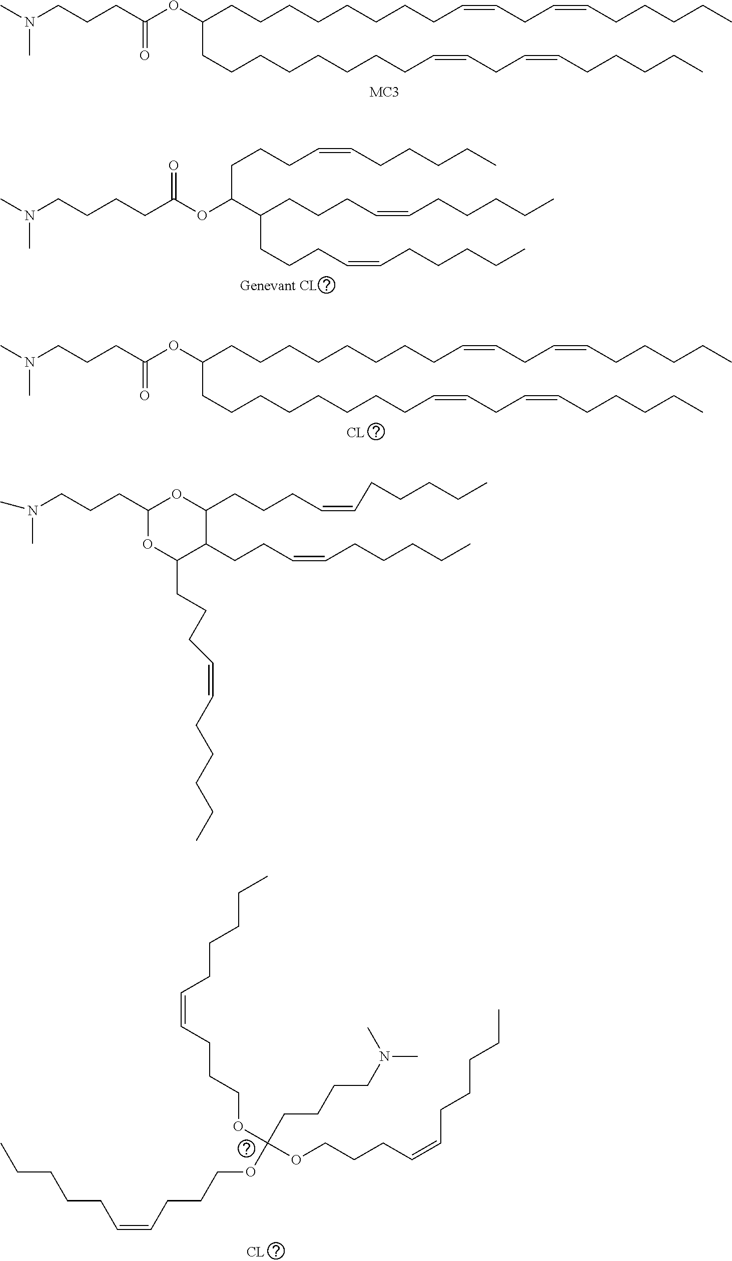

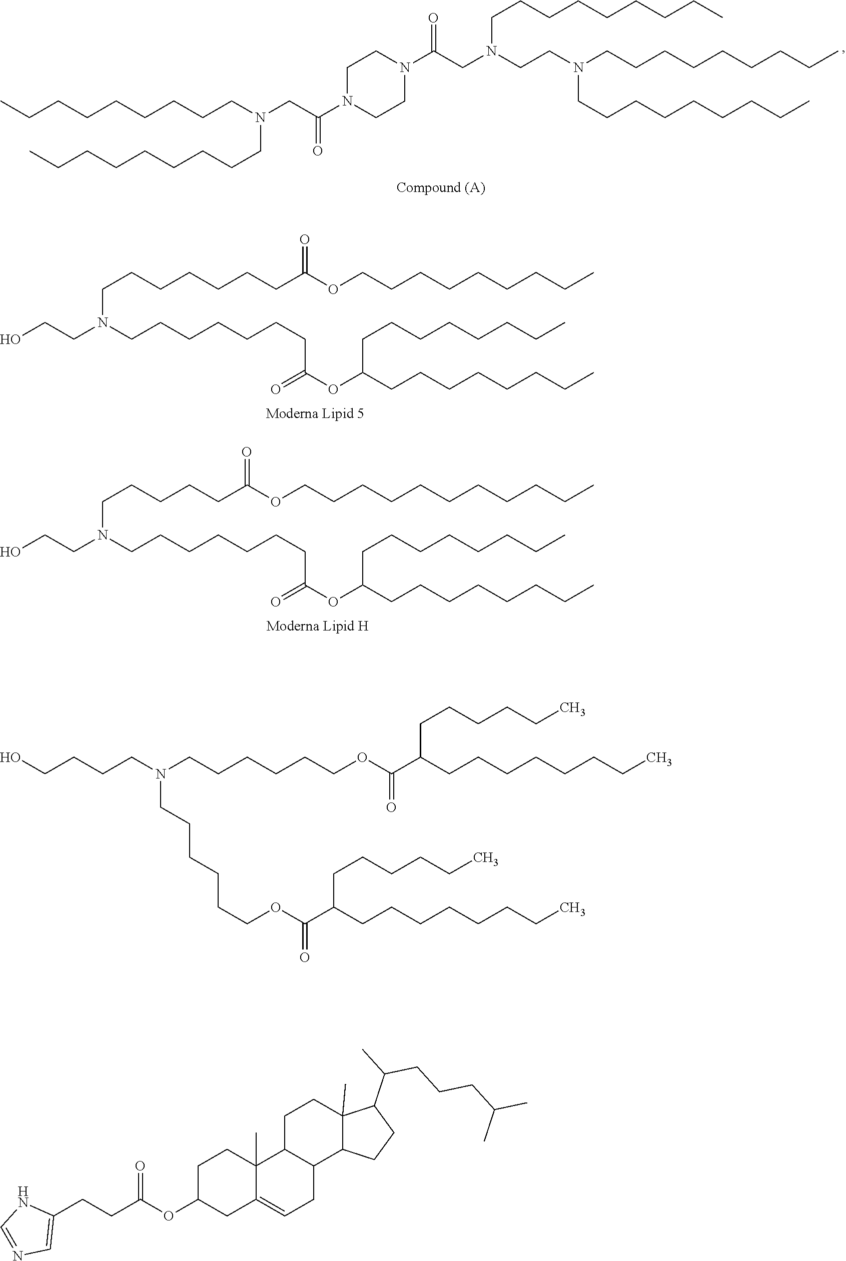 [SMILES: CCCCCCCCCN(CCCCCCCCC)CCN(CCCCCCCCC)CC(=O)N1CCN(C(=O)CN(CCCCCCCCC)CCCCCCCCC)CC1 ; CCCCCCCCCOC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC ; CCCCCCCCC(CCCCCC)C(=O)OCCCCCCN(CCCCO)CCCCCCOC(=O)C(CCCCCC)CCCCCCCC ; CC(C)CCCC(C)C1CCC2C3CC=C4CC(OC(=O)CCc5cnc[nH]5)CCC4(C)C3CCC12C]

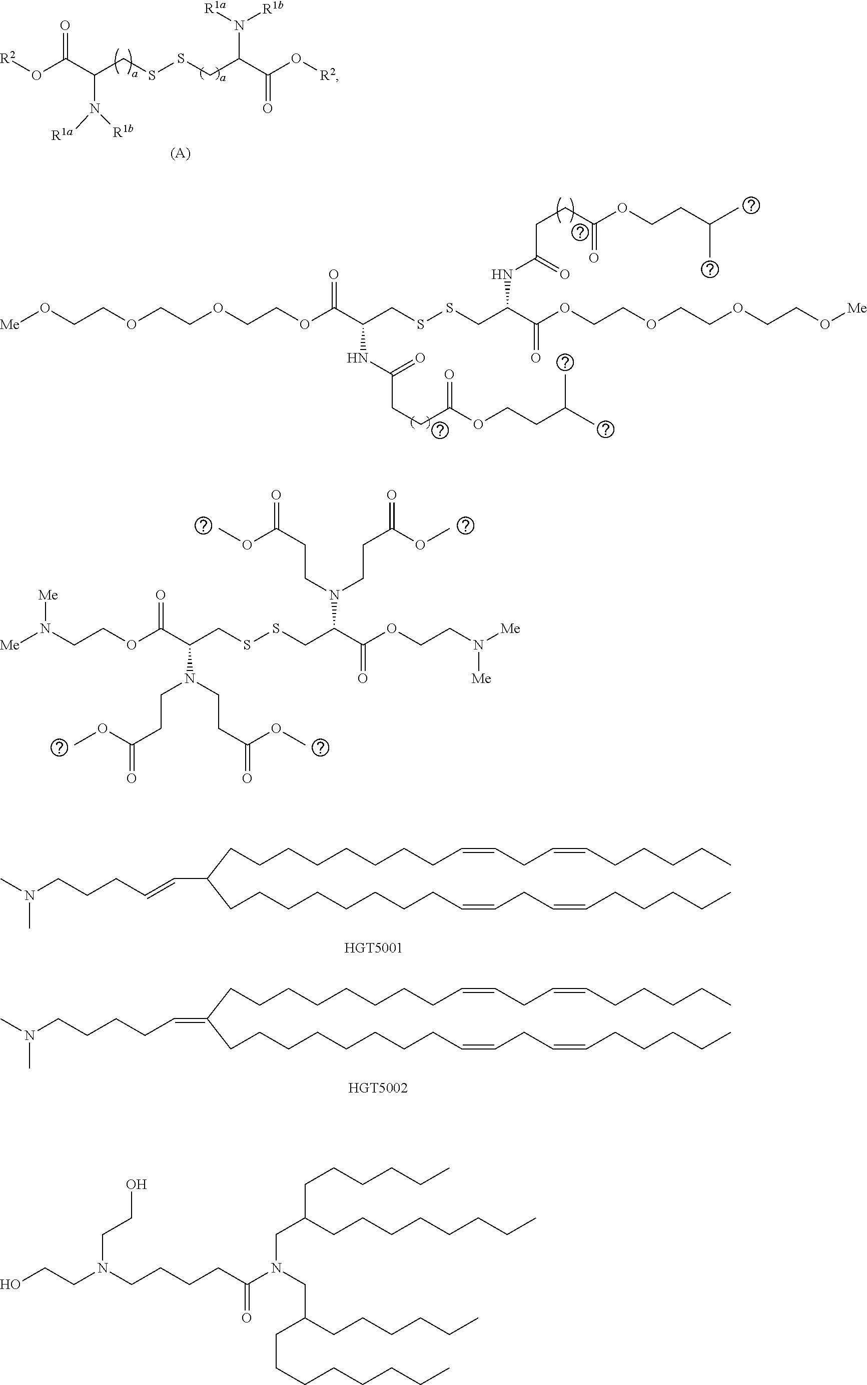

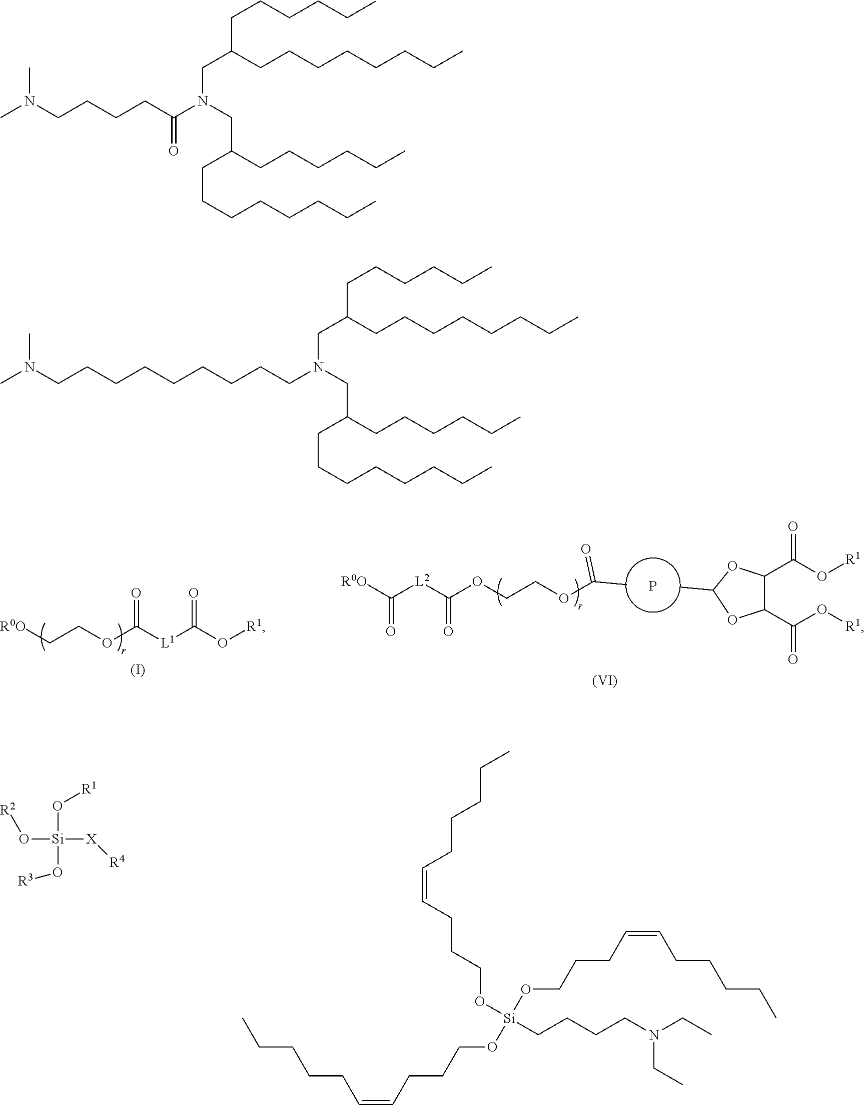

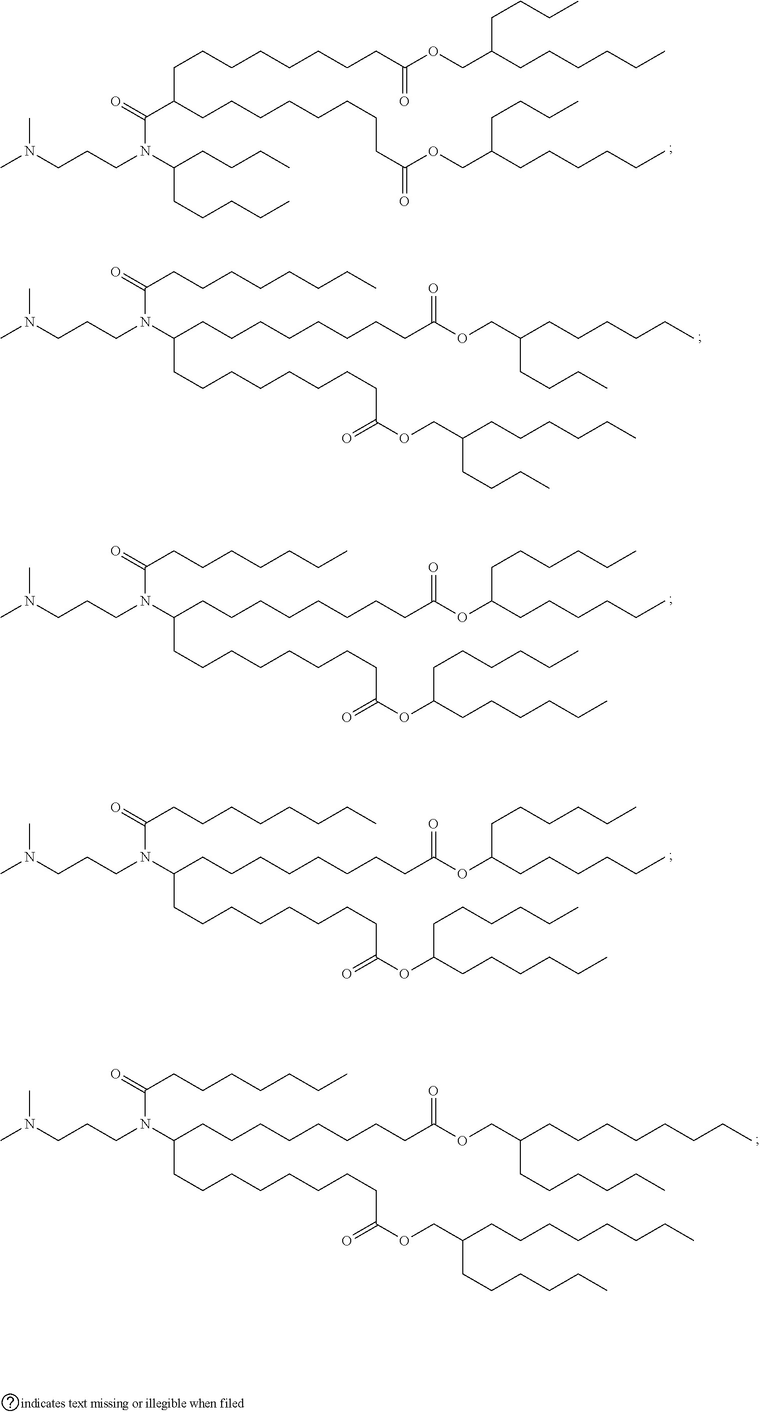

### Neutral Lipids/Phospholipids

In some embodiments, the lipid component further comprises neutral lipids including, but not limited to, a phospholipid selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and mixtures thereof.

### Polymer-Conjugated Lipids

In some embodiments, the lipid component further comprises polymer-conjugated lipids, including, but not limited to, a PEGylated lipid selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG₂₀₀₀-c-DMG, PEG-DLPE, PEG-DMPE, PEG-DPPC, PEG-DMA or a PEG-DSPE lipid.

Non-exhaustive and non-limiting examples of PEG lipids include:

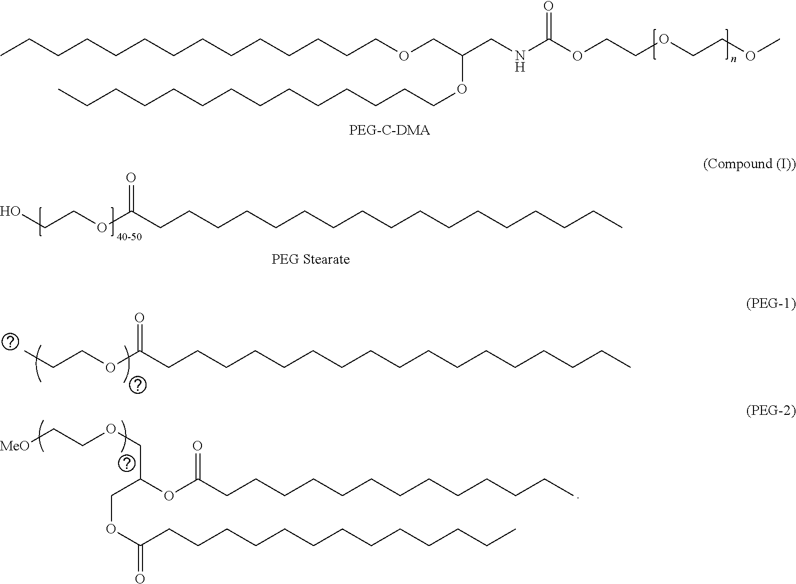

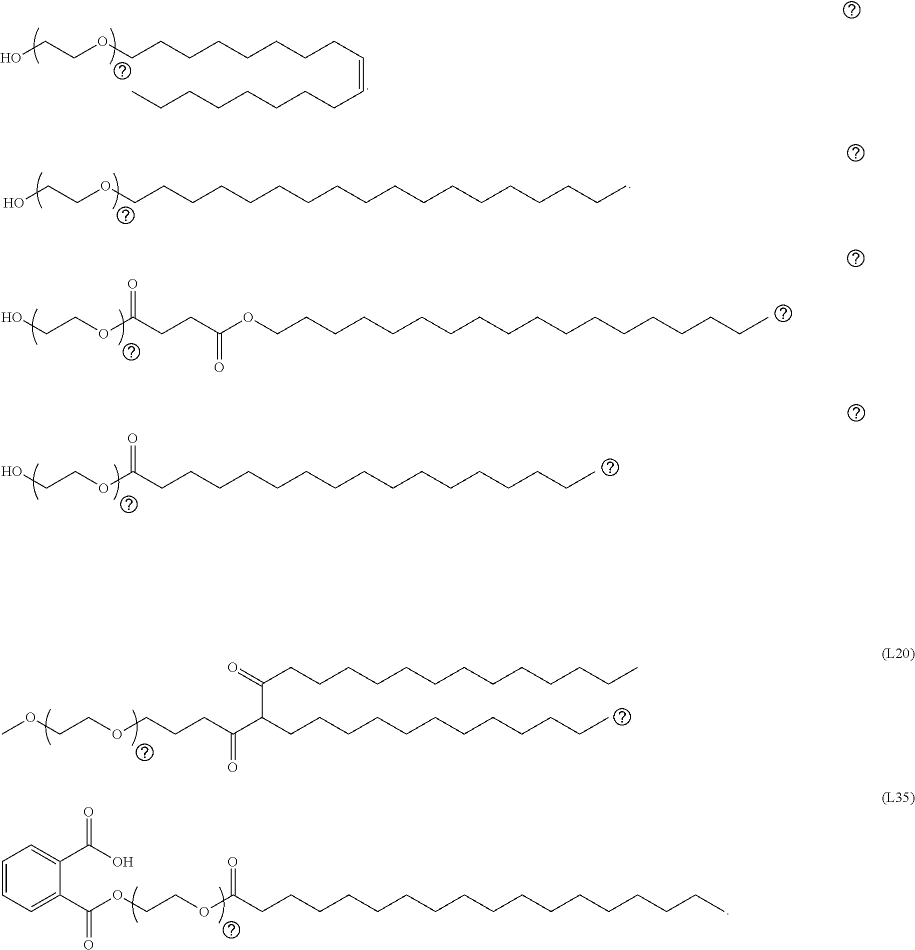

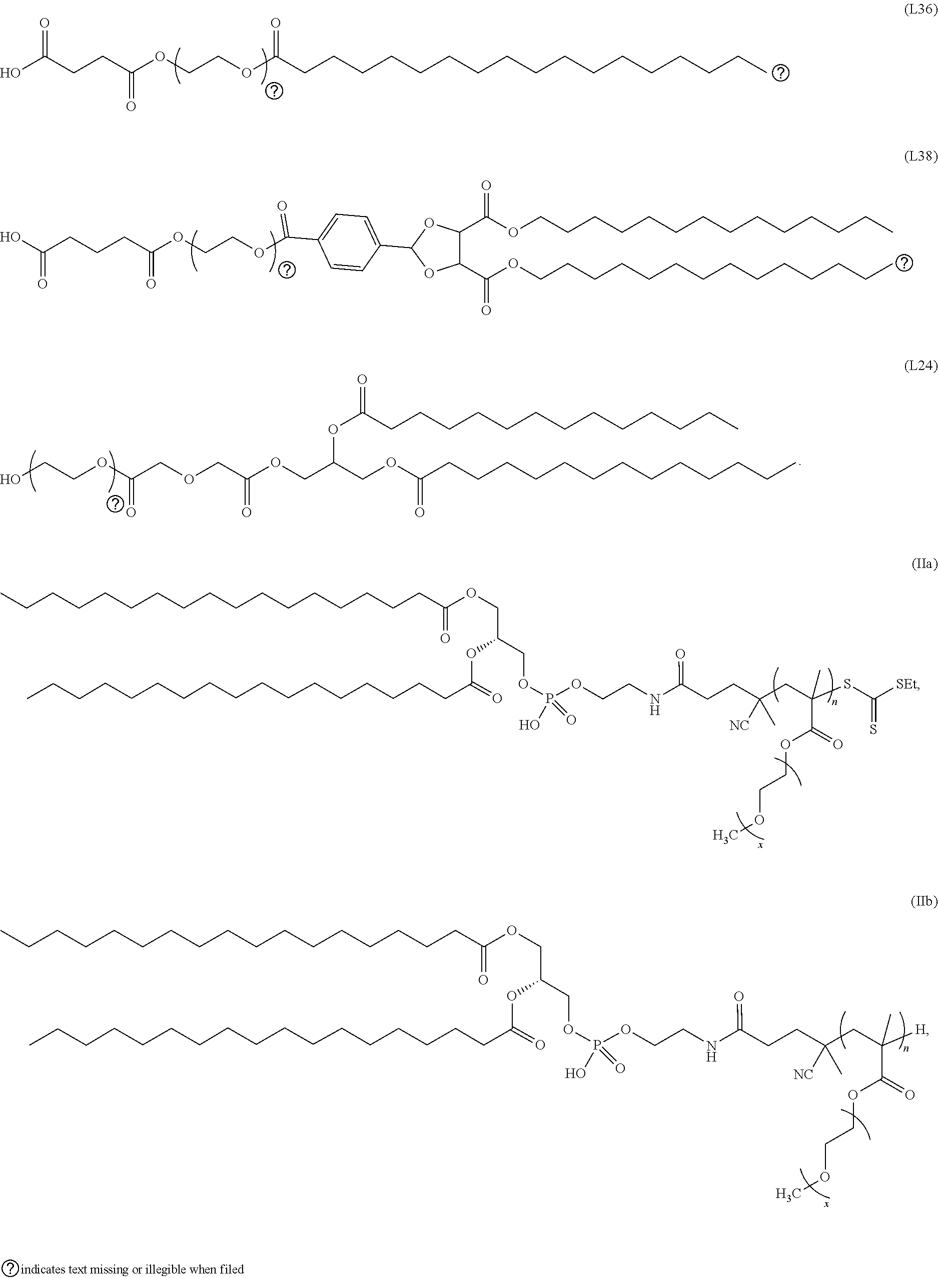

### Structural Lipids/Sterols

In some embodiments, the LNP further comprises a structural component. See generally Patel, S., et al. (2020). Nature Communications, 11(1), 1-13. In some embodiments, the structural component comprises a sterol including, but not limited to, a sterol selected from the group consisting of cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9,11-dehydroergosterol, tomatidine, tomatine, α-tocopherol, and mixtures thereof. In other embodiments, the structural lipid includes cholesterol and a corticosteroid (e.g., prednisolone, dexamethasone, prednisone, and hydrocortisone), or a combination thereof.

Non-exhaustive and non-limiting examples of structural lipids include:

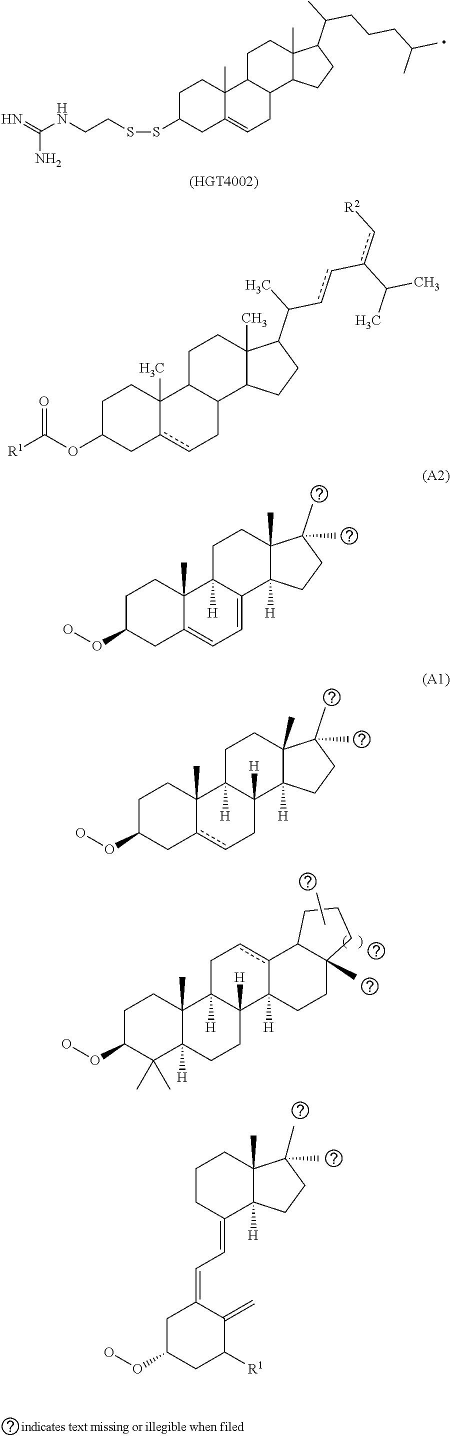

### Formulations

Nanoparticle compositions may include a lipid component and one or more additional components, such as a therapeutic and/or prophylactic. A nanoparticle composition may be designed for one or more specific applications or targets. The elements of a nanoparticle composition may be selected based on a particular application or target, and/or based on the efficacy, toxicity, expense, ease of use, availability, or other feature of one or more elements. Similarly, the particular formulation of a nanoparticle composition may be selected for a particular application or target according to, for example, the efficacy and toxicity of particular combinations of elements.

The lipid component of a nanoparticle composition may include, for example, a cationic lipid, a phospholipid (such as an unsaturated lipid, e.g., DOPE or DSPC), a PEG lipid, and a structural lipid. The elements of the lipid component may be provided in specific fractions.

In some embodiments, the lipid component of a nanoparticle composition includes an ionizable lipid, a phospholipid, a PEG lipid, and a structural lipid. In certain embodiments, the lipid component of the nanoparticle composition includes about 30 mol % to about 60 mol % ionizable lipid, about 0 mol % to about 30 mol % phospholipid, about 0 mol % to about 10 mol % of PEG lipid, and about 17.5 mol % to about 50 mol % structural lipid, provided that the total mol % does not exceed 100%. In some embodiments, the lipid component of the nanoparticle composition includes about 35 mol % to about 55 mol % compound of ionizable lipid, about 5 mol % to about 25 mol % phospholipid, about 0 mol % to about 10 mol % of PEG lipid, and about 30 mol % to about 40 mol % structural lipid. In a particular embodiment, the lipid component includes about 50 mol % said compound, about 10 mol % phospholipid, about 38.5 mol % structural lipid, and about 1.5 mol % of PEG lipid. In another embodiment, the lipid component includes about 40 mol % said compound, about 20 mol % phospholipid, about 38.5 mol % structural lipid, and about 1.5 mol % of PEG lipid. In some embodiments, the phospholipid may be DOPE or DSPC. In other embodiments, the PEG lipid may be PEG-DMG and/or the structural lipid may be cholesterol.

In some embodiments, the ionizable lipids comprise between about 20 and about 60 mol % of the lipid component. In other embodiments, the ionizable lipids comprise between about 35 and about 55 mol % of the lipid component. In various embodiments, the ionizable lipids comprise about 20, 21, 22, 23, 24, 25, 26, 27, 28, 29, 30, 35, 40, 45, 50, 55, or 60 mol % of the lipid component.

In some embodiments, the neutral lipids comprise between about 0 and about 30 mol % of the lipid component. In other embodiments, the neutral lipids comprise between about 5 and about 25 mol % of the lipid component. In various embodiments, the neutral lipids comprise about 1, 2, 3, 4, 5, 6, 7, 8, 9, 10, 11, 12, 13, 14, 15, 16, 17, 18, 19, 20, 25, or 30 mol % of the lipid component.

In some embodiments, the polymer-conjugated lipids comprise between about 0 and about 15 mol % of the lipid component. In other embodiments, the polymer-conjugated lipids comprise between about 0.5 and about 10 mol % of the lipid component. In various embodiments, the polymer-conjugated lipids comprise about 0.5, 1, 1.5, 2, 2.5, 3, 3.5, 4, 4.5, 5, 5.5, 6, 6.5, 7, 7.5, 8, 8.5 9, 9.5, 10, or 15 mol % of the lipid component.

In some embodiments, the structural component comprises about 17.5 mol % to about 50 mol % of the lipid component. In other embodiments, the structural component comprises about 30 to about 40 mol % of the lipid component. In various embodiments, the structural component comprises about 17.5, 20, 22.5, 25, 27.5, 30, 31, 32, 33, 34, 35, 36, 37, 38, 39, 40, 41, 42, 43, 44, 45, 46, 47, 48, 49, or 50 mol % of the lipid component.

The structural component may alternatively be expressed as a ratio relative to the lipid component. In some embodiments, the structural component is in a ratio of about 1:1 with the lipid component (sterol:lipids). In other embodiments, the structural component is in a ratio of about 1:5 with the lipid component (sterol:lipids). In various embodiments, the structural component is in a ratio of about 1:1, 1:2, 1:3, 1:4,1:5, 1:6, 1:7, 1:8, 1:9, 1:10, 1:15, 1:20, or 1:25 with the lipid component (sterol:lipids).

Nanoparticle compositions may be designed for one or more specific applications or targets. For example, a nanoparticle composition may be designed to deliver a therapeutic and/or prophylactic such as an RNA to a particular cell, tissue, organ, or system or group thereof in a mammal's body. Physiochemical properties of nanoparticle compositions may be altered in order to increase selectivity for particular bodily targets. For instance, particle sizes may be adjusted based on the fenestration sizes of different organs. The therapeutic and/or prophylactic included in a nanoparticle composition may also be selected based on the desired delivery target or tar-gets. For example, a therapeutic and/or prophylactic may be selected for a particular indication, condition, disease, or disorder and/or for delivery to a particular cell, tissue, organ, or system or group thereof (e.g., localized or specific delivery). In certain embodiments, a nanoparticle com-position may include an mRNA encoding a polypeptide of interest capable of being translated within a cell to produce the polypeptide of interest. Such a composition may be designed to be specifically delivered to a particular organ. In some embodiments, a composition may be de-signed to be specifically delivered to a mammalian joint.

The amount of a therapeutic and/or prophylactic in a nanoparticle composition may depend on the size, composition, desired target and/or application, or other properties of the nanoparticle composition as well as on the properties of the therapeutic and/or prophylactic. For example, the amount of an RNA useful in a nanoparticle composition may depend on the size, sequence, and other characteristics of the RNA. The relative amounts of a therapeutic and/or prophylactic and other elements (e.g., lipids) in a nanoparticle composition may also vary. In some embodiments, the wt/wt ratio of the lipid component to a therapeutic and/or prophylactic in a nanoparticle composition may be from about 5:1 to about 60:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, and 60:1. For example, the wt/wt ratio of the lipid component to a therapeutic and/or prophylactic may be from about 10:1 to about 40:1. In certain embodiments, the wt/wt ratio is about 20:1. The amount of a therapeutic and/or prophylactic in a nanoparticle composition may, for example, be measured using absorption spectroscopy (e.g., ultraviolet-visible spectroscopy).

In some embodiments, the therapeutic and/or prophylactic comprises a nucleic acid component. In some embodiments, the nucleic acid component comprises RNA including, but not limited to, RNA selected from the group consisting of messenger RNA (mRNA), CRISPR RNA (crRNA), tracrRNA, single-guide RNA (sgRNA), short interfering RNA (siRNA), antisense oligonucleotides (ASO), and mixtures thereof. In other embodiments, the nucleic acid component comprises DNA including, but not limited to, DNA selected from the group consisting of linear DNA, plasmid DNA, antisense oligonucleotide, and mixtures thereof.

In some embodiments, a nanoparticle composition includes one or more RNAs, and the one or more RNAs, lipids, and amounts thereof may be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof may be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio may be from about 2:1 to about 8:1. In other embodiments, the N:P ratio is from about 5:1 to about 8:1. For example, the N:P ratio may be about 5.0:1, about 5.5:1, about 5.67:1, about 6.0:1, about 6.5:1, or about 7.0:1. For example, the N:P ratio may be about 5.67:1.

In some embodiments, the nucleic acid component is comprised of a modified nucleic acid. For example, an RNA may be a modified RNA. That is, an RNA may include one or more nucleobases, nucleosides, nucleotides, or linkers that are non-naturally occurring. A “modified” species may also be referred to herein as an “altered” species. Species may be modified or altered chemically, structurally, or functionally. For example, a modified nucleobase species may include one or more substitutions that are not naturally occurring.

In certain embodiments, the present disclosure comprises methods for treating back or spine conditions or disorders. In other embodiments, the present disclosure comprises methods for treating discogenic disorders. In some embodiments, the present disclosure comprises methods for treating localized nociception, inflammation, or morphological changes associated with back or spine conditions or disorders in a subject in need thereof, the method comprising administering a therapeutically effective amount of a CRISPR-Cas composition encapsulated within or associated with a lipid nanoparticle (LNP), wherein the composition comprises one or more non-naturally occurring polynucleotides encoding a Cas9 protein and at least one sgRNA. In some embodiments, LNPs are administered intradiscally. In other embodiments, LNPs are administered epidurally. In some embodiments, LNPs are administered peridiscally. In some embodiments, LNPs are administered perivertebrally.

### Physical Properties

The characteristics of a nanoparticle composition may depend on the components thereof. For example, a nanoparticle composition including cholesterol as a structural lipid may have different characteristics than a nanoparticle composition that includes a different structural lipid. Similarly, the characteristics of a nanoparticle composition may depend on the absolute or relative amounts of its components. For instance, a nanoparticle composition including a higher molar fraction of a phospholipid may have different characteristics than a nanoparticle composition including a lower molar fraction of a phospholipid. Characteristics may also vary depending on the method and conditions of preparation of the nanoparticle composition.

Nanoparticle compositions may be characterized by a variety of methods. For example, microscopy (e.g., transmission electron microscopy or scanning electron microscopy) may be used to examine the morphology and size distribution of a nanoparticle composition. Dynamic light scattering or potentiometry (e.g., potentiometric titrations) may be used to measure zeta potentials. Dynamic light scattering may also be utilized to determine particle sizes. Instruments such as the Zetasizer Nano ZS (Malvern Instruments Ltd, Malvern, Worcestershire, UK) may also be used to measure multiple characteristics of a nanoparticle composition, such as particle size, polydispersity index, and zeta potential.

The mean size of a nanoparticle composition may be between 10 nm and 1 micrometer, e.g., measured by dynamic light scattering (DLS). For example, the mean size may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the mean size of a nanoparticle composition may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In certain embodiments, the mean size of a nanoparticle composition may be from about 70 nm to about 100 nm. In a particular embodiment, the mean size may be about 80 nm. In other embodiments, the mean size may be about 100 nm.

A nanoparticle composition may be relatively homogenous. A polydispersity index may be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition may be from about 0.10 to about 0.20.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a therapeutic and/or prophylactic describes the amount of therapeutic and/or prophylactic that is encapsulated or otherwise associated with a nanoparticle composition after preparation, relative to the initial amount provided. The encapsulation efficiency is desirably high (e.g., close to 100%). The encapsulation efficiency may be measured, for example, by comparing the amount of therapeutic and/or prophylactic in a solution containing the nanoparticle composition before and after breaking up the nanoparticle composition with one or more organic solvents or detergents. Fluorescence may be used to measure the amount of free therapeutic and/or prophylactic (e.g., RNA) in a solution. For the nanoparticle compositions described herein, the encapsulation efficiency of a therapeutic and/or prophylactic may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In certain embodiments, the encapsulation efficiency may be at least 90%.

A nanoparticle composition may optionally comprise one or more coatings. For example, a nanoparticle composition may be formulated in a capsule, film, or tablet having a coating. A capsule, film, or tablet including a composition described herein may have any useful size, tensile strength, hardness, or density.

Unless otherwise defined, all technical and scientific terms used herein have the same meaning as commonly understood by one of ordinary skill in the art to which this disclosure belongs. Although methods and materials similar or equivalent to those described herein may be used in the practice or testing of the present disclosure, suitable methods and materials are described below. All publications, patent applications, patents, and other references mentioned herein are incorporated by reference in their entirety. In addition, the materials, methods, and examples are illustrative only and not intended to be limiting.

In some embodiments, the CRISPR gene-editing system comprises one or more RNA-containing compositions. In some embodiments, the CRISPR gene-editing system further comprises one or more nanoparticles. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanoparticles are lipid nanoparticles (LNP).

In some embodiments, the CRISPR gene-editing system comprises one or more LNPs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNPs comprises a first plurality of LNP encapsulating the RNA-guided nuclease or a nucleic acid encoding an RNA-guided nuclease and a second plurality of LNP encapsulating the at least one guide RNA or a nucleic acid encoding at least one guide RNA.

In some embodiments, the one or more LNP comprises a component selected from the group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), a lipid including a cyclic amine group, and a mixture thereof.

In some embodiments, the one or more LNP comprises a component selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin (SM), and a mixture thereof.

In some embodiments, the one or more LNP comprises a component selected from the group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA, a PEG-DSPE lipid, and a mixture thereof.

In some embodiments, the one or more LNP comprises a component selected from the group consisting of a cholesterol, fecosterol, stigmasterol, stigmastanol, sitosterol, β-sitosterol, lupeol, betulin, ursolic acid, oleanolic acid, campesterol, fucosterol, brassicasterol, ergosterol, 9, 11-dehydroergosterol, tomatidine, tomatine, α-tocopherol, and a mixture thereof.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In some aspect, use of the CRISPR gene-editing system further comprising one or more LNPs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to. Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more LNPs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### B. Virus-Like Particles

In one aspect, the present disclosure encompasses means for delivering a CRISPR gene-editing system to a mammalian cell via a virus-like particle (VLP). In some embodiments, a CRISPR gene-editing system is delivered by a VLP. Without wishing to be bound by any particular theory, in certain embodiments, nucleic acids, when present in the particle, are resistant in aqueous solution to degradation with a nuclease. In other embodiments, proteins are protected from protease degradation while present in the particle. In some embodiments, proteins and nucleic acids encapsulated by VLPs are capable of penetrating the cellular plasma membrane.

In some embodiments, the CRISPR gene-editing system comprises one or more RNA-containing compositions. In some embodiments, the CRISPR gene-editing system further comprises one or more VLPs. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, the CRISPR gene-editing system comprises one or more virus-like particles collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more virus-like particles comprises a first plurality of virus-like particles encapsulating the RNA-guided nuclease or a nucleic acid encoding an RNA-guided nuclease and a second plurality of virus-like particles encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more VLPs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more VLPs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### C. Miscellaneous Modes of Delivery

### 1. Liposomes

In some embodiments, nucleic acids encoding a CRISPR gene-editing system targeting a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A, and combinations thereof (e.g., Cas9 or gRNA) are entrapped in liposomes bearing positive charges on their surface (e.g., lipofectins), which can be tagged with antibodies against cell surface antigens of the target cells. These delivery vehicles can also be used to deliver Cas9 protein/gRNA complexes.

In some embodiments, the CRISPR gene-editing system comprises one or more RNA-containing compositions. In some embodiments, the CRISPR gene-editing system further comprises one or more liposomes. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA).

In some embodiments, wherein the composition comprises one or more liposomes collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more liposomes comprises a first plurality of liposomes encapsulating the RNA-guided nuclease or a nucleic acid encoding an RNA-guided nuclease and a second plurality of liposomes encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more liposomes to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more liposomes to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, colorectal cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### 2. Lipid Nanocrystals (LNC)

In one aspect, the In one aspect, the present disclosure encompasses means for delivering a CRISPR gene-editing system to a mammalian cell via a lipid nanocrystal (LNC). In some embodiments, a CRISPR gene-editing system is delivered by a LNC. Without wishing to be bound by any particular theory, in certain embodiments, nucleic acids, when present in the nanocrystal, are resistant in aqueous solution to degradation with a nuclease. In other embodiments, proteins are protected from protease degradation while present in the nanocrystal. In some embodiments, proteins and nucleic acids encapsulated by nanocrystal are capable of penetrating the cellular plasma membrane.

In some embodiments, the CRISPR gene-editing system comprises one or more RNA-containing compositions. In some embodiments, the CRISPR gene-editing system further comprises one or more nanocrystals. In some embodiments, said one or more RNA-containing compositions comprises a guide RNA. In some embodiments, said one or more RNA-containing compositions comprises an mRNA. In some embodiments, said one or more RNA-containing compositions comprises an RNP (e.g., Cas9 and a guide RNA). In some embodiments, said one or more nanocrystals are lipid nanocrystals (LNC).

In some embodiments, the CRISPR gene-editing system comprises one or more LNCs collectively encapsulating (i) the RNA-guided nuclease or the nucleic acid encoding the RNA-guided nuclease and (ii) the at least one guide RNA or the nucleic acid encoding the at least one guide RNA. In some embodiments, the one or more LNCs comprises a first plurality of LNC encapsulating the RNA-guided nuclease or a nucleic acid encoding an RNA-guided nuclease and a second plurality of LNC encapsulating the at least one guide RNA or a nucleic acid encoding at least one guide RNA.

In some embodiments, use of the CRISPR gene-editing system further comprising one or more LNPs to target a gene selected from IL1R1, IL1RAP, TGFBR1, TGFBR2, IL6R, IL6ST, TNFRSF1A, TNFRSF1B, TNFRSF3, TNFRSF4, TNFRSF11A, and combinations thereof is therapeutic.

In one aspect, use of the CRISPR gene-editing system further comprising one or more LNCs to target TGFBR1 and/or TGFBR2 is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, Loeys-Dietz Syndrome, osteoarthritis, Marfan syndrome, aortic aneurysm (e.g., familial thoracic 3 aortic aneurysm), craniofacial abnormalities, and combinations thereof. In other embodiments, use of the system treats neoplastic diseases, conditions, and illnesses, including, but not limited to, pancreatic cancer, multiple self-healing squamous epithelioma (Ferguson-Smith disease), gastrointestinal stromal tumors (GIST), hereditary nonpolyposis colorectal cancer (Lynch Syndrome), metastatic colorectal carcinoma, bone neoplasms, anaplastic carcinoma, spindle-cell carcinoma, lung neoplasms, brain neoplasms, and combinations thereof.

In some embodiments, the CRISPR gene-editing system further comprising one or more LNCs to target IL1R1 and/or IL1RAP is therapeutic. In some embodiments, use of the system treats one or more joint disease or illness or one or more back or spine conditions or disorders. In some embodiments, use of the system treats one or more musculoskeletal diseases, conditions, and illnesses, including, but not limited to, rheumatoid arthritis, gout, osteoarthritis, osteoporosis, intervertebral disc disease (IVDD), psoriatic arthritis, arthritis, polymyositis, proliferative synovitis, bone neoplasms, sarcoid myopathy, cortex bone disorders, idiopathic scoliosis, tendinopathy, myofibrillar myopathy, enthesis-related arthritis, ankylosing spondylitis, degenerative polyarthritis, arthropathy, osteitis deformans, prolapsed lumbar disc, polymyositis ossificans, idiopathic polymyositis, Luft Disease, adult-onset Still's Disease, osteoarthritis deformans, Bachet's Disease, and combinations thereof. In other embodiments, use of the system treats one or more neoplastic diseases, conditions, and illnesses, including, but not limited to, osteosarcoma, multiple myeloma, lymphoma (e.g., B-cell or cutaneous T-cell lymphoma), leukemia, thyroid carcinoma, glioma, renal cell carcinoma, chondrosarcoma, glioblastoma, melanoma, neuroblastoma, polycystic ovary syndrome, Kaposi sarcoma, squamous cell carcinoma, leiomyosarcoma, malignant peripheral nerve sheath tumor, Ewing's sarcoma, esophageal neoplasms, colon cancer, lung cancer, breast cancer, pancreatic cancer, stomach cancer, epithelial ovarian cancer, cholectoral cancer, liver cancer, skin cancer, prostate cancer, cervical cancer, ovarian cancer, bladder cancer, oral cavity cancer, and combinations thereof. In other embodiments, use of the system treats one or more inflammatory diseases, conditions, and illnesses, including, but not limited to, autoinflammatory disease (AID), cryopyrin-associated periodic syndrome (CAPS), systemic lupus erythematosus (SLE), inflammatory bowel disease (IBD) and combinations thereof. In some embodiments, use of the system treats one or more cardiac diseases, conditions, and illnesses, including, but not limited to, myocardial infarction, heart failure (e.g., refractory or acute decompensated heart failure), arrythmias, pericarditis (e.g., refractory idiopathic pericarditis), myocarditis, sepsis-induced cardiomyopathy, atherosclerosis, coronary artery disease and combinations thereof. In some embodiments, use of the system treats one or more neurological diseases, conditions, and illnesses, including, but not limited to, acute thrombotic stroke, epilepsy, multiple sclerosis, Alzheimer's Disease and combinations thereof. In some embodiments, use of the system treats one or more ophthalmic diseases, conditions, and illnesses, including, but not limited to, uveitis, scleritis, Sjogren asthenia, dry eye and combinations thereof. In some embodiments, use of the system treats one or more diseases, conditions, and illnesses, including, but not limited to, chronic kidney disease, Type 2 diabetes, gastroesophageal reflux disease (GERD), non-HP-associated peptic ulcer disease, and pulmonary fibrosis.

### VII. Pharmaceutical Compositions

In one aspect, the present disclosure encompasses pharmaceutical compositions comprising a CRISPR gene-editing system for treatment of a mammal in need thereof. In some embodiments, the CRISPR gene-editing system targets a gene selected from IL1R1, IL1R2, IL1RAP, IL1RL1, TGFBR1, TGFBR2, IL6R, IL6ST, TNFRSF1A, TNFRSF1B, TNFRSF3, TNFRSF4, TNFRSF11A, and combinations thereof. In some embodiments, the mammal is selected from a human, a dog, a horse, and a cat.

### A. IL1A

In some embodiments, the disclosure provides one or more pharmaceutical compositions targeting an IL1A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL1A gene, and an LNP system selected from any one of LNP001 to LNP240. In some embodiments, the at least one guide RNA has a crRNA sequence selected from SEQ ID NOs: 769-786. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL1A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### B. IL1B

In some embodiments, the disclosure provides one or more pharmaceutical compositions targeting an IL1B gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL1B gene, and an LNP system selected from any one of LNP001 to LNP240. In some embodiments, the at least one guide RNA has a crRNA sequence selected from SEQ ID NOs: 787-805. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL1B gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### C. IL1R1

In some embodiments, the disclosure provides one or more pharmaceutical compositions targeting an IL1R1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL1R1 gene, and an LNP system selected from any one of LNP001 to LNP240. In some embodiments, the at least one guide RNA has a crRNA sequence selected from SEQ ID NOs: 806-839. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL1R1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### D. IL1RAP

In some embodiments, the disclosure provides one or more pharmaceutical compositions targeting an IL1RAP gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL1RAP gene, and an LNP system selected from any one of LNP001 to LNP240. In some embodiments, the at least one guide RNA has a crRNA sequence selected from SEQ ID NOs: 840-887. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL1RAP gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### E. IL4

In some embodiments, the disclosure provides one or more pharmaceutical compositions targeting an IL4 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL4 gene, and an LNP system selected from any one of LNP001 to LNP240. In some embodiments, the at least one guide RNA has a crRNA sequence selected from SEQ ID NOs: 888-911. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL4 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### F. IL6R

In some embodiments, the disclosure provides one or more pharmaceutical compositions targeting an IL6R gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL6R gene, and an LNP system selected from any one of LNP001 to LNP240. In some embodiments, the at least one guide RNA has a crRNA sequence selected from SEQ ID NOs: 929-963. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL6R gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### G. IL6ST

In some embodiments, the disclosure provides one or more pharmaceutical compositions targeting an IL6ST gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL6ST gene, and an LNP system selected from any one of LNP001 to LNP240. In some embodiments, the at least one guide RNA has a crRNA sequence selected from SEQ ID NOs: 964-990. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL6ST gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### H. TNFRSF1A

In some embodiments, the disclosure provides one or more pharmaceutical compositions targeting an TNFRSF1A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNFRSF1A gene, and an LNP system selected from any one of LNP001 to LNP240. In some embodiments, the at least one guide RNA has a crRNA sequence selected from SEQ ID NOs: 2575-2622. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNFRSF1A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### I. TNFRSF1B

In some embodiments, the disclosure provides one or more pharmaceutical compositions targeting an TNFRSF1B gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNFRSF1B gene, and an LNP system selected from any one of LNP001 to LNP240. In some embodiments, the at least one guide RNA has a crRNA sequence selected from SEQ ID NOs: 2623-2670. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNFRSF1B gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### J. TNFRSF3

In some embodiments, the disclosure provides one or more pharmaceutical compositions targeting an TNFRSF3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNFRSF3 gene, and an LNP system selected from any one of LNP001 to LNP240. In some embodiments, the at least one guide RNA has a crRNA sequence selected from SEQ ID NOs: 1664-1684. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNFRSF3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### K. TNFRSF4

In some embodiments, the disclosure provides one or more pharmaceutical compositions targeting an TNFRSF4 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNFRSF4 gene, and an LNP system selected from any one of LNP001 to LNP240. In some embodiments, the at least one guide RNA has a crRNA sequence selected from SEQ ID NOs: 1685-1711. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNFRSF4 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### L. TNFRSF11A

In some embodiments, the disclosure provides one or more pharmaceutical compositions targeting an TNFRSF11A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNFRSF11A gene, and an LNP system selected from any one of LNP001 to LNP240. In some embodiments, the at least one guide RNA has a crRNA sequence selected from SEQ ID NOs: 1711-1759. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNFRSF11A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### M. ADAM17

In some embodiments, the disclosure provides one or more pharmaceutical compositions targeting an ADAM17 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an ADAM17 gene, and an LNP system selected from any one of LNP001 to LNP240. In some embodiments, the at least one guide RNA has a crRNA sequence selected from SEQ ID NOs: 1-48. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an ADAM17 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### N. ADAMTS1

In some embodiments, the disclosure provides one or more pharmaceutical compositions targeting an ADAMTS1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an ADAMTS1 gene, and an LNP system selected from any one of LNP001 to LNP240. In some embodiments, the at least one guide RNA has a crRNA sequence selected from SEQ ID NOs: 49-96. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an ADAMTS1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### O. ADAMTS5

In some embodiments, the disclosure provides one or more pharmaceutical compositions targeting an ADAMTS1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an ADAMTS1 gene, and an LNP system selected from any one of LNP001 to LNP240. In some embodiments, the at least one guide RNA has a crRNA sequence selected from SEQ ID NOs: 97-144. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an ADAMTS1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### P. ADM

In some embodiments, the disclosure provides one or more pharmaceutical compositions targeting an ADM gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an ADM gene, and an LNP system selected from any one of LNP001 to LNP240. In some embodiments, the at least one guide RNA has a crRNA sequence selected from SEQ ID NOs: 145-192. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an ADM gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### Q. ATP1A1

In some embodiments, the disclosure provides one or more pharmaceutical compositions targeting an ATP1A1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an ATP1A1 gene, and an LNP system selected from any one of LNP001 to LNP240. In some embodiments, the at least one guide RNA has a crRNA sequence selected from SEQ ID NOs: 193-240. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an ATP1A1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### R. BDNF

In some embodiments, the disclosure provides one or more pharmaceutical compositions targeting an BDNF gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an BDNF gene, and an LNP system selected from any one of LNP001 to LNP240. In some embodiments, the at least one guide RNA has a crRNA sequence selected from SEQ ID NOs: 241-281. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an BDNF gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### S. CALCA

In some embodiments, the disclosure provides one or more pharmaceutical compositions targeting an CALCA gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CALCA gene, and an LNP system selected from any one of LNP001 to LNP240. In some embodiments, the at least one guide RNA has a crRNA sequence selected from SEQ ID NOs: 282-301. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CALCA gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### T. CALCB

In some embodiments, the disclosure provides one or more pharmaceutical compositions targeting an CALCB gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CALCB gene, and an LNP system selected from any one of LNP001 to LNP240. In some embodiments, the at least one guide RNA has a crRNA sequence selected from SEQ ID NOs: 302-318. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CALCB gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### U. CALCRL

In some embodiments, the disclosure provides one or more pharmaceutical compositions targeting an CALCRL gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CALCRL gene, and an LNP system selected from any one of LNP001 to LNP240. In some embodiments, the at least one guide RNA has a crRNA sequence selected from SEQ ID NOs: 319-340. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CALCRL gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### V. CCL2

In some embodiments, the disclosure provides one or more pharmaceutical compositions targeting an CCL2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCL2 gene, and an LNP system selected from any one of LNP001 to LNP240. In some embodiments, the at least one guide RNA has a crRNA sequence selected from SEQ ID NOs: 341-357. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCL2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### W. CCL3

In some embodiments, the disclosure provides one or more pharmaceutical compositions targeting an CCL3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCL3 gene, and an LNP system selected from any one of LNP001 to LNP240. In some embodiments, the at least one guide RNA has a crRNA sequence selected from SEQ ID NOs: 358-374. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCL3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### X. CCL5

In some embodiments, the disclosure provides one or more pharmaceutical compositions targeting an CCL5 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCL5 gene, and an LNP system selected from any one of LNP001 to LNP240. In some embodiments, the at least one guide RNA has a crRNA sequence selected from SEQ ID NOs: 375-391. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCL5 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### Y. CCL7

In some embodiments, the disclosure provides one or more pharmaceutical compositions targeting an CCL7 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCL7 gene, and an LNP system selected from any one of LNP001 to LNP240. In some embodiments, the at least one guide RNA has a crRNA sequence selected from SEQ ID NOs: 392-408. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCL7 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### Z. CCL20

In some embodiments, the disclosure provides one or more pharmaceutical compositions targeting an CCL20 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCL20 gene, and an LNP system selected from any one of LNP001 to LNP240. In some embodiments, the at least one guide RNA has a crRNA sequence selected from SEQ ID NOs: 409-425. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCL20 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### AA. CCN2

In some embodiments, the disclosure provides one or more pharmaceutical compositions targeting an CCN2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCN2 gene, and an LNP system selected from any one of LNP001 to LNP240. In some embodiments, the at least one guide RNA has a crRNA sequence selected from SEQ ID NOs: 426-473. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCN2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### BB. CCR7

In some embodiments, the disclosure provides one or more pharmaceutical compositions targeting an CCR7 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CCR7 gene, and an LNP system selected from any one of LNP001 to LNP240. In some embodiments, the at least one guide RNA has a crRNA sequence selected from SEQ ID NOs: 474-517. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CCR7 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### CC. CRCP

In some embodiments, the disclosure provides one or more pharmaceutical compositions targeting an CRCP gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CRCP gene, and an LNP system selected from any one of LNP001 to LNP240. In some embodiments, the at least one guide RNA has a crRNA sequence selected from SEQ ID NOs: 518-534. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CRCP gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### DD. CXCL1

In some embodiments, the disclosure provides one or more pharmaceutical compositions targeting an CXCL1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL1 gene, and an LNP system selected from any one of LNP001 to LNP240. In some embodiments, the at least one guide RNA has a crRNA sequence selected from SEQ ID NOs: 535-551. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCL1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### EE. CXCL2

In some embodiments, the disclosure provides one or more pharmaceutical compositions targeting an CXCL2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL2 gene, and an LNP system selected from any one of LNP001 to LNP240. In some embodiments, the at least one guide RNA has a crRNA sequence selected from SEQ ID NOs: 552-568. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCL2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### FF. CXCL3

In some embodiments, the disclosure provides one or more pharmaceutical compositions targeting an CXCL3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL3 gene, and an LNP system selected from any one of LNP001 to LNP240. In some embodiments, the at least one guide RNA has a crRNA sequence selected from SEQ ID NOs: 569-585. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the pharmaceutical composition targeting an CXCL3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### GG. CXCL5

In some embodiments, the disclosure provides one or more pharmaceutical compositions targeting an CXCL5 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL5 gene, and an LNP system selected from any one of LNP001 to LNP240. In some embodiments, the at least one guide RNA has a crRNA sequence selected from SEQ ID NOs: 586-602. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCL5 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### HH. CXCL6

In some embodiments, the disclosure provides one or more pharmaceutical compositions targeting an CXCL6 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL6 gene, and an LNP system selected from any one of LNP001 to LNP240. In some embodiments, the at least one guide RNA has a crRNA sequence selected from SEQ ID NOs: 603-619. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCL6 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### II. CXCL8

In some embodiments, the disclosure provides one or more pharmaceutical compositions targeting an CXCL8 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCL8 gene, and an LNP system selected from any one of LNP001 to LNP240. In some embodiments, the at least one guide RNA has a crRNA sequence selected from SEQ ID NOs: 620-636. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCL8 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### JJ. CXCR1

In some embodiments, the disclosure provides one or more pharmaceutical compositions targeting an CXCR1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCR1 gene, and an LNP system selected from any one of LNP001 to LNP240. In some embodiments, the at least one guide RNA has a crRNA sequence selected from SEQ ID NOs: 637-655. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCR1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### KK. CXCR2

In some embodiments, the disclosure provides one or more pharmaceutical compositions targeting an CXCR2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an CXCR2 gene, and an LNP system selected from any one of LNP001 to LNP240. In some embodiments, the at least one guide RNA has a crRNA sequence selected from SEQ ID NOs: 656-672. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an CXCR2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### LL. FGF2

In some embodiments, the disclosure provides one or more pharmaceutical compositions targeting an FGF2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an FGF2 gene, and an LNP system selected from any one of LNP001 to LNP240. In some embodiments, the at least one guide RNA has a crRNA sequence selected from SEQ ID NOs: 673-720. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an FGF2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### MM. FGFR1

In some embodiments, the disclosure provides one or more pharmaceutical compositions targeting an FGFR1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an FGFR1 gene, and an LNP system selected from any one of LNP001 to LNP240. In some embodiments, the at least one guide RNA has a crRNA sequence selected from SEQ ID NOs: 721-768. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an FGFR1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### NN. IL10

In some embodiments, the disclosure provides one or more pharmaceutical compositions targeting an IL10 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL10 gene, and an LNP system selected from any one of LNP001 to LNP240. In some embodiments, the at least one guide RNA has a crRNA sequence selected from SEQ ID NOs: 991-1007. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL10 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### OO. IL10RA

In some embodiments, the disclosure provides one or more pharmaceutical compositions targeting an IL10RA gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL10RA gene, and an LNP system selected from any one of LNP001 to LNP240. In some embodiments, the at least one guide RNA has a crRNA sequence selected from SEQ ID NOs: 1008-1055. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL10RA gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### PP. IL10RB

In some embodiments, the disclosure provides one or more pharmaceutical compositions targeting an IL10RB gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL10RB gene, and an LNP system selected from any one of LNP001 to LNP240. In some embodiments, the at least one guide RNA has a crRNA sequence selected from SEQ ID NOs: 1056-1082. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL10RB gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### QQ. IL13

In some embodiments, the disclosure provides one or more pharmaceutical compositions targeting an IL13 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL13 gene, and an LNP system selected from any one of LNP001 to LNP240. In some embodiments, the at least one guide RNA has a crRNA sequence selected from SEQ ID NOs: 1083-1104. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL13 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### RR. IL13RA1

In some embodiments, the disclosure provides one or more pharmaceutical compositions targeting an IL13RA1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL13RA1 gene, and an LNP system selected from any one of LNP001 to LNP240. In some embodiments, the at least one guide RNA has a crRNA sequence selected from SEQ ID NOs: 1105-1130. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL13RA1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### SS. IL13RA2

In some embodiments, the disclosure provides one or more pharmaceutical compositions targeting an IL13RA2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL13RA2 gene, and an LNP system selected from any one of LNP001 to LNP240. In some embodiments, the at least one guide RNA has a crRNA sequence selected from SEQ ID NOs: 1131-1147. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL13RA2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### TT. IL17A

In some embodiments, the disclosure provides one or more pharmaceutical compositions targeting an IL17A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL17A gene, and an LNP system selected from any one of LNP001 to LNP240. In some embodiments, the at least one guide RNA has a crRNA sequence selected from SEQ ID NOs: 1148-1173. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL17A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### UU. IL17RA

In some embodiments, the disclosure provides one or more pharmaceutical compositions targeting an IL17RA gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL17RA gene, and an LNP system selected from any one of LNP001 to LNP240. In some embodiments, the at least one guide RNA has a crRNA sequence selected from SEQ ID NOs: 1174-1221. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL17RA gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### VV. IL18

In some embodiments, the disclosure provides one or more pharmaceutical compositions targeting an IL18 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL18 gene, and an LNP system selected from any one of LNP001 to LNP240. In some embodiments, the at least one guide RNA has a crRNA sequence selected from SEQ ID NOs: 1222-1238. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL18 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### WW. IL18R1

In some embodiments, the disclosure provides one or more pharmaceutical compositions targeting an IL18R1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL18R1 gene, and an LNP system selected from any one of LNP001 to LNP240. In some embodiments, the at least one guide RNA has a crRNA sequence selected from SEQ ID NOs: 1239-1262. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL18R1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### XX. IL18RAP

In some embodiments, the disclosure provides one or more pharmaceutical compositions targeting an IL18RAP gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an IL18RAP gene, and an LNP system selected from any one of LNP001 to LNP240. In some embodiments, the at least one guide RNA has a crRNA sequence selected from SEQ ID NOs: 1263-1310. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an IL18RAP gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### YY. MMP1

In some embodiments, the disclosure provides one or more pharmaceutical compositions targeting an MMP1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP1 gene, and an LNP system selected from any one of LNP001 to LNP240. In some embodiments, the at least one guide RNA has a crRNA sequence selected from SEQ ID NOs: 1311-1343. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### ZZ. MMP2

In some embodiments, the disclosure provides one or more pharmaceutical compositions targeting an MMP2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP2 gene, and an LNP system selected from any one of LNP001 to LNP240. In some embodiments, the at least one guide RNA has a crRNA sequence selected from SEQ ID NOs: 1344-1391. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### AAA. MMP3

In some embodiments, the disclosure provides one or more pharmaceutical compositions targeting an MMP3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP3 gene, and an LNP system selected from any one of LNP001 to LNP240. In some embodiments, the at least one guide RNA has a crRNA sequence selected from SEQ ID NOs: 1392-1417. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### BBB. MMP7

In some embodiments, the disclosure provides one or more pharmaceutical compositions targeting an MMP7 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP7 gene, and an LNP system selected from any one of LNP001 to LNP240. In some embodiments, the at least one guide RNA has a crRNA sequence selected from SEQ ID NOs: 1418-1436. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP7 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### CCC. MMP8

In some embodiments, the disclosure provides one or more pharmaceutical compositions targeting an MMP8 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP8 gene, and an LNP system selected from any one of LNP001 to LNP240. In some embodiments, the at least one guide RNA has a crRNA sequence selected from SEQ ID NOs: 1437-1474. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP8 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### DDD. MMP10

In some embodiments, the disclosure provides one or more pharmaceutical compositions targeting an MMP10 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP10 gene, and an LNP system selected from any one of LNP001 to LNP240. In some embodiments, the at least one guide RNA has a crRNA sequence selected from SEQ ID NOs: 1475-1497. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP10 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### EEE. MMP12

In some embodiments, the disclosure provides one or more pharmaceutical compositions targeting an MMP12 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP12 gene, and an LNP system selected from any one of LNP001 to LNP240. In some embodiments, the at least one guide RNA has a crRNA sequence selected from SEQ ID NOs: 1498-1541. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP12 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### FFF. MMP13

In some embodiments, the disclosure provides one or more pharmaceutical compositions targeting an MMP13 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MMP13 gene, and an LNP system selected from any one of LNP001 to LNP240. In some embodiments, the at least one guide RNA has a crRNA sequence selected from SEQ ID NOs: 1542-1568. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MMP13 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### GGG. MRGPRX2

In some embodiments, the disclosure provides one or more pharmaceutical compositions targeting an MRGPRX2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an MRGPRX2 gene, and an LNP system selected from any one of LNP001 to LNP240. In some embodiments, the at least one guide RNA has a crRNA sequence selected from SEQ ID NOs: 1569-1585. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an MRGPRX2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### HHH. NGF

In some embodiments, the disclosure provides one or more pharmaceutical compositions targeting an NGF gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NGF gene, and an LNP system selected from any one of LNP001 to LNP240. In some embodiments, the at least one guide RNA has a crRNA sequence selected from SEQ ID NOs: 1586-1628. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NGF gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### III. NGFR

In some embodiments, the disclosure provides one or more pharmaceutical compositions targeting an NGFR gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NGFR gene, and an LNP system selected from any one of LNP001 to LNP240. In some embodiments, the at least one guide RNA has a crRNA sequence selected from SEQ ID NOs: 1629-1676. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NGFR gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### JJJ. NTF3

In some embodiments, the disclosure provides one or more pharmaceutical compositions targeting an NTF3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NTF3 gene, and an LNP system selected from any one of LNP001 to LNP240. In some embodiments, the at least one guide RNA has a crRNA sequence selected from SEQ ID NOs: 1677-1724. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NTF3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### KKK. NTF4

In some embodiments, the disclosure provides one or more pharmaceutical compositions targeting an NTF4 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NTF4 gene, and an LNP system selected from any one of LNP001 to LNP240. In some embodiments, the at least one guide RNA has a crRNA sequence selected from SEQ ID NOs: 1725-1746. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NTF4 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### LLL. NTRK1

In some embodiments, the disclosure provides one or more pharmaceutical compositions targeting an NTRK1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NTRK1 gene, and an LNP system selected from any one of LNP001 to LNP240. In some embodiments, the at least one guide RNA has a crRNA sequence selected from SEQ ID NOs: 1747-1794. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NTRK1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### MMM. NTRK2

In some embodiments, the disclosure provides one or more pharmaceutical compositions targeting an NTRK2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an NTRK2 gene, and an LNP system selected from any one of LNP001 to LNP240. In some embodiments, the at least one guide RNA has a crRNA sequence selected from SEQ ID NOs: 1795-1842. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an NTRK2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### NNN. RAMP1

In some embodiments, the disclosure provides one or more pharmaceutical compositions targeting an RAMP1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an RAMP1 gene, and an LNP system selected from any one of LNP001 to LNP240. In some embodiments, the at least one guide RNA has a crRNA sequence selected from SEQ ID NOs: 1843-1859. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an RAMP1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### OOO. SCN1A

In some embodiments, the disclosure provides one or more pharmaceutical compositions targeting an SCN1A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN1A gene, and an LNP system selected from any one of LNP001 to LNP240. In some embodiments, the at least one guide RNA has a crRNA sequence selected from SEQ ID NOs: 1860-1907. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN1A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### PPP. SCN2A

In some embodiments, the disclosure provides one or more pharmaceutical compositions targeting an SCN2A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN2A gene, and an LNP system selected from any one of LNP001 to LNP240. In some embodiments, the at least one guide RNA has a crRNA sequence selected from SEQ ID NOs: 1908-1955. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN2A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### QQQ. SCN3A

In some embodiments, the disclosure provides one or more pharmaceutical compositions targeting an SCN3A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN3A gene, and an LNP system selected from any one of LNP001 to LNP240. In some embodiments, the at least one guide RNA has a crRNA sequence selected from SEQ ID NOs: 1956-2003. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN3A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### RRR. SCN4A

In some embodiments, the disclosure provides one or more pharmaceutical compositions targeting an SCN4A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN4A gene, and an LNP system selected from any one of LNP001 to LNP240. In some embodiments, the at least one guide RNA has a crRNA sequence selected from SEQ ID NOs: 2004-2051. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN4A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### SSS. SCN5A

In some embodiments, the disclosure provides one or more pharmaceutical compositions targeting an SCN5A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN5A gene, and an LNP system selected from any one of LNP001 to LNP240. In some embodiments, the at least one guide RNA has a crRNA sequence selected from SEQ ID NOs: 2052-2099. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN5A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### TTT. SCN8A

In some embodiments, the disclosure provides one or more pharmaceutical compositions targeting an SCN8A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN8A gene, and an LNP system selected from any one of LNP001 to LNP240. In some embodiments, the at least one guide RNA has a crRNA sequence selected from SEQ ID NOs: 2100-2147. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN8A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### UUU. SCN9A

In some embodiments, the disclosure provides one or more pharmaceutical compositions targeting an SCN9A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN9A gene, and an LNP system selected from any one of LNP001 to LNP240. In some embodiments, the at least one guide RNA has a crRNA sequence selected from SEQ ID NOs: 2148-2195. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN9A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### VVV. SCN10A

In some embodiments, the disclosure provides one or more pharmaceutical compositions targeting an SCN10A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN10A gene, and an LNP system selected from any one of LNP001 to LNP240. In some embodiments, the at least one guide RNA has a crRNA sequence selected from SEQ ID NOs: 2196-2243. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN10A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### WWW. SCN11A

In some embodiments, the disclosure provides one or more pharmaceutical compositions targeting an SCN11A gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an SCN11A gene, and an LNP system selected from any one of LNP001 to LNP240. In some embodiments, the at least one guide RNA has a crRNA sequence selected from SEQ ID NOs: 2244-2291. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an SCN11A gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### XXX. TAC1

In some embodiments, the disclosure provides one or more pharmaceutical compositions targeting an TAC1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TAC1 gene, and an LNP system selected from any one of LNP001 to LNP240. In some embodiments, the at least one guide RNA has a crRNA sequence selected from SEQ ID NOs: 2292-2308. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TACT gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### YYY. TAC3

In some embodiments, the disclosure provides one or more pharmaceutical compositions targeting an TAC3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TAC3 gene, and an LNP system selected from any one of LNP001 to LNP240. In some embodiments, the at least one guide RNA has a crRNA sequence selected from SEQ ID NOs: 2309-2325. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TAC3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### ZZZ. TACR1

In some embodiments, the disclosure provides one or more pharmaceutical compositions targeting an TACR1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TACR1 gene, and an LNP system selected from any one of LNP001 to LNP240. In some embodiments, the at least one guide RNA has a crRNA sequence selected from SEQ ID NOs: 2326-2373. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TACR1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### AAAA. TACR2

In some embodiments, the disclosure provides one or more pharmaceutical compositions targeting an TACR2 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TACR2 gene, and an LNP system selected from any one of LNP001 to LNP240. In some embodiments, the at least one guide RNA has a crRNA sequence selected from SEQ ID NOs: 2374-2421. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TACR2 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### BBBB. TACR3

In some embodiments, the disclosure provides one or more pharmaceutical compositions targeting an TACR3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TACR3 gene, and an LNP system selected from any one of LNP001 to LNP240. In some embodiments, the at least one guide RNA has a crRNA sequence selected from SEQ ID NOs: 2422-2469. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TACR3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### CCCC. TIMP1

In some embodiments, the disclosure provides one or more pharmaceutical compositions targeting an TIMP1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TIMP1 gene, and an LNP system selected from any one of LNP001 to LNP240. In some embodiments, the at least one guide RNA has a crRNA sequence selected from SEQ ID NOs: 2470-2509. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TIMP1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### DDDD. TIMP3

In some embodiments, the disclosure provides one or more pharmaceutical compositions targeting an TIMP3 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TIMP3 gene, and an LNP system selected from any one of LNP001 to LNP240. In some embodiments, the at least one guide RNA has a crRNA sequence selected from SEQ ID NOs: 2510-2557. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TIMP3 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### EEEE. TNF

In some embodiments, the disclosure provides one or more pharmaceutical compositions targeting an TNF gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an TNF gene, and an LNP system selected from any one of LNP001 to LNP240. In some embodiments, the at least one guide RNA has a crRNA sequence selected from SEQ ID NOs: 2558-2574. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an TNF gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### FFFF. YAP1

In some embodiments, the disclosure provides one or more pharmaceutical compositions targeting an YAP1 gene, the composition comprising a plurality of LNPs, wherein the LNPs comprise an RNA-guided nuclease or a nucleic acid encoding an RNA-guided nuclease and at least one guide RNA or a nucleic acid encoding at least one guide RNA targeting an YAP1 gene, and an LNP system selected from any one of LNP001 to LNP240. In some embodiments, the at least one guide RNA has a crRNA sequence selected from SEQ ID NOs: 2671-2718. In some embodiments, the LNP system comprises the steroid component in a molar ratio from about 36 to about 46, or from about 38 to about 42. In some embodiments, the LNP system comprises the steroid component in a molar ratio of about 37, about 38, about 39, about 40, about 41, or about 42. In some embodiments, the steroid component comprises cholesterol. In some embodiments, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000. In some embodiments, the plurality of LNPs have an average diameter between about 60 nm and about 120 nm. In some embodiments, the concentration of LNPs in the pharmaceutical composition is between 0.001 and 3 mg/ml. In some embodiments, the pharmaceutical composition targeting an YAP1 gene is formulated for treating one or more disorders selected from (i) a joint disease or illness (e.g., arthritis, post-traumatic arthritis, osteoarthritis, rheumatoid arthritis, psoriatic arthritis, lupus, ankylosing spondylitis, reactive arthritis, septic arthritis, juvenile idiopathic arthritis, fibromyalgia, crystal arthropathies, gout, pseudogout, or CPPD disease/chondrocalcinosis), (ii) a spinal condition or disorder (e.g. discogenic disease, low back pain, neck pain, lumbar pain, degenerative disc disease, annual ligament tear, herniated disc, spinal facet joint arthritis, intervertebral disc disease, spondylosis, painful scoliosis, or spinal stenosis), (iii) a musculoskeletal disorder (e.g. fibrosis, scarring, infectious intermittent joint effusion, spondylarthritis, plantar fasciitis, degenerative polyarthritis, hemophilic arthropathy, inflammatory myopathy with abundant macrophages, or polymyositis), and/or any combinations thereof.

### VIII. Administration Routes

The methods and compositions herein described encompass the use of pharmaceutical compositions comprising a CRISPR gene-editing system as an active ingredient.

Depending on the method/route of administration, pharmaceutical dosage forms come in several types. These include many kinds of liquid, solid, and semisolid dosage forms. Common pharmaceutical dosage forms include pill, tablet, or capsule, drink or syrup, and natural or herbal form such as plant or food of sorts, among many others. Notably, the route of administration (ROA) for drug delivery is dependent on the dosage form of the substance in question. A liquid pharmaceutical dosage form is the liquid form of a dose of a chemical compound used as a drug or medication intended for administration or consumption.

As described below, a composition of the present disclosure can be delivered to a subject subcutaneously (e.g., intra-articular or intradiscal injection), dermally (e.g., transdermally via patch), and/or via implant. Exemplary pharmaceutical dosage forms include, e.g., pills, osmotic delivery systems, elixirs, emulsions, hydrogels, suspensions, syrups, capsules, tablets, orally dissolving tablets (ODTs), gel capsules, thin films, adhesive topical patches, lollipops, lozenges, chewing gum, dry powder inhalers (DPIs), vaporizers, nebulizers, metered dose inhalers (MDIs), ointments, transdermal patches, intradermal implant.

As used herein, “dermal delivery” or “dermal administration” can refer to a route of administration wherein the pharmaceutical dosage form is taken to, or through, the dermis (i.e., layer of skin between the epidermis (with which it makes up the cutis) and subcutaneous tissues). “Subcutaneous delivery” can refer to a route of administration wherein the pharmaceutical dosage form is to or beneath the subcutaneous tissue layer.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

Therapeutic compounds can be prepared with carriers that will protect the therapeutic compounds against rapid elimination from the body, such as a controlled release formulation, including implants and microencapsulated delivery systems. Biodegradable, biocompatible polymers can be used, such as collagen, ethylene vinyl acetate, polyanhydrides (e.g., poly[1,3-bis(carboxyphenoxy)propane-co-sebacic-acid](PCPP-SA) matrix, fatty acid dimer-sebacic acid (FAD-SA) copolymer, poly(lactide-co-glycolide)), polyglycolic acid, collagen, polyorthoesters, polyethylene glycol-coated liposomes, hyaluronic acid and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811. Semisolid, gelling, soft-gel, or other formulations (including controlled release) can be used, e.g., when administration to a surgical site is desired. Methods of making such formulations are known in the art and can include the use of biodegradable, biocompatible polymers. See, e.g., Sawyer et al., Yale J Biol Med. 2006 December; 79(3-4): 141-152.

The pharmaceutical compositions described herein may be included in a container, kit, pack, or dispenser together with instructions for administration.

### A. Systemic Administration

In some embodiments, a pharmaceutical composition comprising a CRISPR gene-editing system is administered systemically to a mammal in need thereof. In some embodiments, the composition is formulated for intravenous injection. In some embodiments, the composition is formulated for oral administration. In some embodiments, the composition is formulated for parenteral administration.

### B. Local Administration

In some embodiments, a pharmaceutical composition comprising a CRISPR gene-editing system is administered locally to a mammal in need thereof. In some embodiments, the local administration is an intra-articular injection. In some embodiments, the composition is formulated for intradiscal injection. In some embodiments, the composition is formulated for epidural injection. In some embodiments, the composition is formulated for peridiscal injection. In some embodiments, the composition is formulated for perivertebral injection. In some embodiments, composition is formulated for administration to the facet joints of the spine.

In some embodiments, a pharmaceutical composition comprising a CRISPR gene-editing system is administered locally to a mammal in need thereof during a surgical procedure. In some embodiments, a pharmaceutical composition comprising a CRISPR gene-editing system is administered locally to a mammal in need thereof 1, 2, 3, 4, 5, 6, 7, 8, 9, 10, 20, or 30 days after a surgical procedure.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Design of Crispr/Cas9 Guide RNAs Targeting cIL1A and cIL1B

Publicly accessible genomes (human, hg38; dog, CanFam3.1), collapsed gene models (merged Ensembl/Havana), tissue-specific exon expression (gtexportal.org) and various gRNA models were then used to select two to five individual crRNA sequences per gene, targeting canine and human interleukin-1 alpha (IL1A) and interleukin-1 beta (IL1B). The following gRNA design rules were applied:
1. The gRNA target region was limited to the first 5-50% of the coding sequence (CDS).
2. Single gRNAs were ranked according to maximal on-target editing using Azimuth 2.0 model (10.1038/nbt.3437) and minimal off-target editing using Cutting Frequencing Determination (CFD) (10.1038/nbt.3437) and the specificity score from Hsu et al. (10.1038/nbt.2647).
3. Highly ranked sgRNA with high frameshift frequencies (>75%) and uniform DNA repair outcomes (>0.48) as predicted by inDelphi (10.1038/s41586-018-0686-x) were selected for in vitro synthesis.

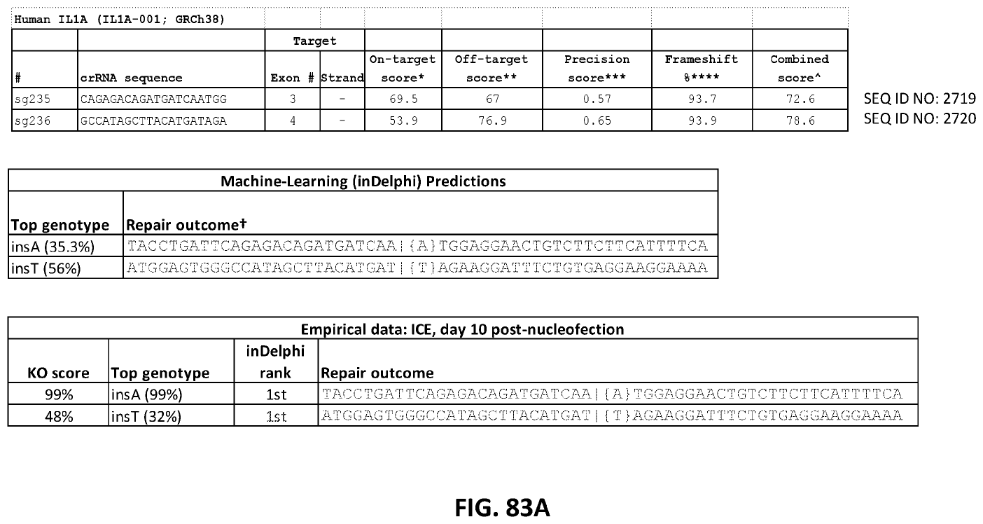

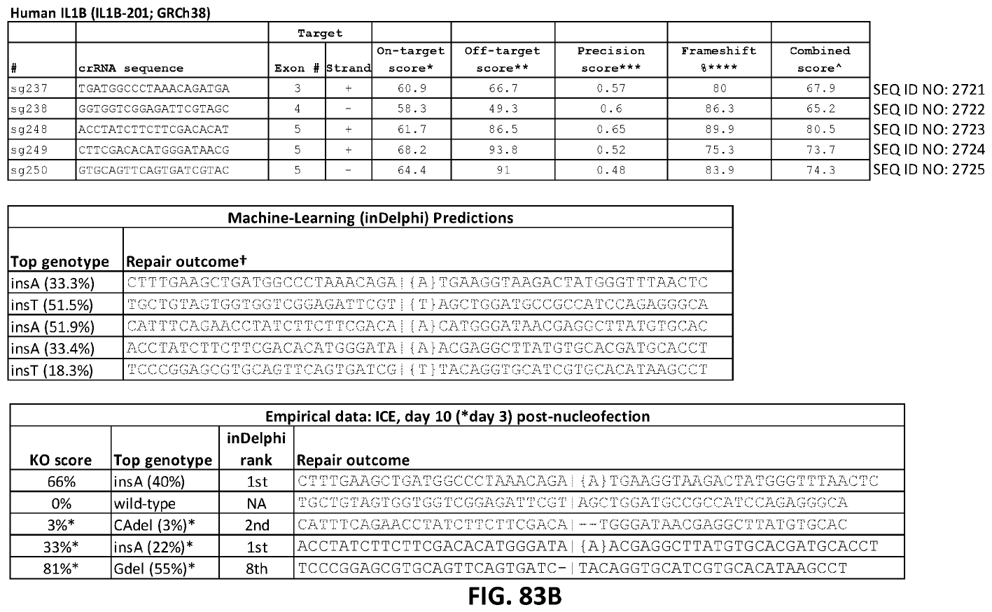

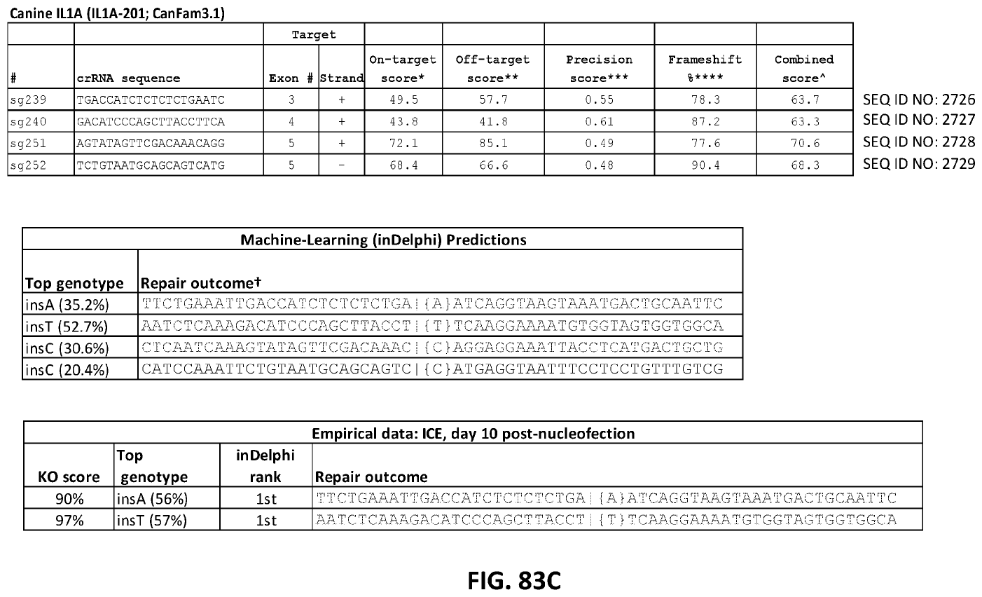

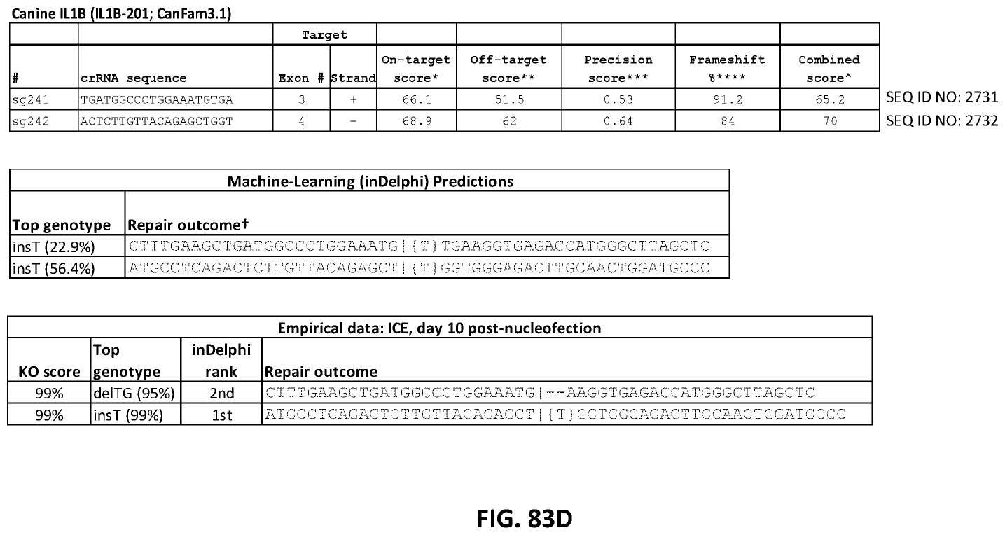

Using these selection criteria, crRNA guide sequences targeting different exons of the respective target genes were selected for further investigation. Specifically, as shown in FIG. 83A, sg235 (SEQ ID NO:2719) and sg236 (SEQ ID NO:2720) targeting exons 3 and 4 of the hIL1A gene were selected. Likewise, as shown in FIG. 83B, sg237 (SEQ ID NO:2721), sg238 (SEQ ID NO: 2722), sg248 (SEQ ID NO: 2723), sg249 (SEQ ID NO: 2724), and sg250 (SEQ ID NO: 2725) targeting exons 3, 4, and 5 of the hIL1B gene were selected. As shown in FIG. 83C, sg239 (SEQ ID NO: 2726), sg240 (SEQ ID NO: 2727), sg251 (SEQ ID NO:2728), and sg252 (SEQ ID NO:2729) targeting exons 3, 4, and 5 of the cIL1A gene were selected. Likewise, as shown in FIG. 83D, sg241 (SEQ ID NO: 2730) and sg242 (SEQ ID NO: 2731) targeting exons 3 and 4 of the cIL1B gene were selected.

sgRNAs, fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with 4 μg Cas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (ab1) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

Results of the empirical experiments and machine-learning prediction of gene editing using the selected guide sequences are shown in FIG. 83 and support the overall design method for various gene targets in any well-annotated genome.

### Example 2: Functional Impact of IL1A/IL1B Editing

The sgRNAs with the highest knockout (KO) scores from Example 8 (i.e., the highest frameshift frequency) were used to generate double IL-1α/IL-1β knock out (KO) cells. Specifically, human chondrocytes were edited to achieve >99% IL-1α KO using crRNA sequence CAGAGACAGAUGAUCAAUGG and 67% IL-1β KO using crRNA sequence GUGCAGUUCAGUGAUCGUAC. Canine chondrocytes were edited to achieve 97% IL-1α KO using crRNA sequence GACAUCCCAGCUUACCUUCA and 99% IL-1β KO using crRNA sequence ACUCUUGUUACAGAGCUGGU.

Canine chondrocytes (Catalog Cn402K-05), human chondrocytes (Catalog 402-05a) and human fibroblast-like synovial cells (Catalog 408-05a) were purchased as frozen stocks (5×10\hat{ }5 cells) from Cell Applications, Inc., San Diego, CA. Chondrocytes were cultured in growth medium consisting of DMEM/Ham's F12 (Gibco, Catalog 21331-020) supplemented with 20% (v/v) untreated FBS (Gibco, Catalog 10270-106) and 1×GlutaMAX (Gibco, Catalog 35050-038). Synovial cells were cultured in growth medium consisting of DMEM (Gibco, Catalog 11960-044), 10% non-treated FBS (Gibco, Catalog 10270-106) and 1×GlutaMAX (Gibco, Catalog 35050-038). Cells were confirmed as being negative for Mycoplasma spp. and subjected to STR profiling prior to use. For electroporation and subculture, cells were dissociated using 0.25% trypsin (Gibco, Catalog 25200056). Trypsin was quenched with 9 volumes of growth medium and cells were spun at 1,000 g to remove the supernatant.

Interleukin-1 (IL-1) release was induced by challenging sub-confluent monolayers of cells (edited or wild-type non-edited) with lipopolysaccharide (LPS). In brief, non-edited (control) and double IL-1α/IL-1β KO (edited) human or canine chondrocytes were seeded at density of approximately 5×10⁴ cells per well in 24-well plates. After 24-48 hours, the medium was replaced with fresh, serum-free medium containing either LPS (50 μg/ml) or PBS vehicle and the plates returned to the incubator. Plates were harvested after 6 and 24 hours for the determination of IL-1 release. Media were snap-frozen in liquid nitrogen and stored at −20° C. until they were assayed.

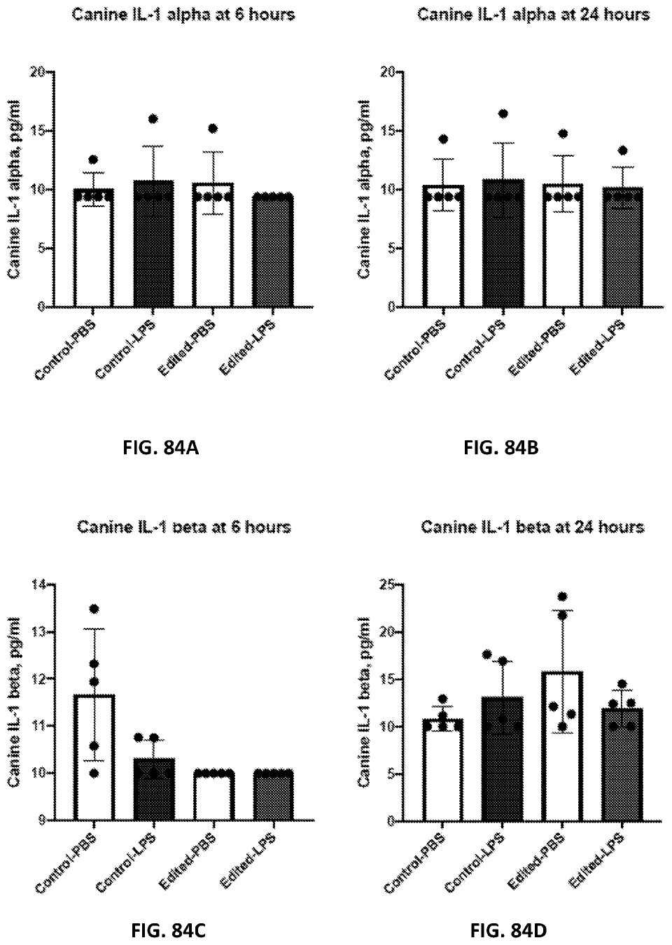

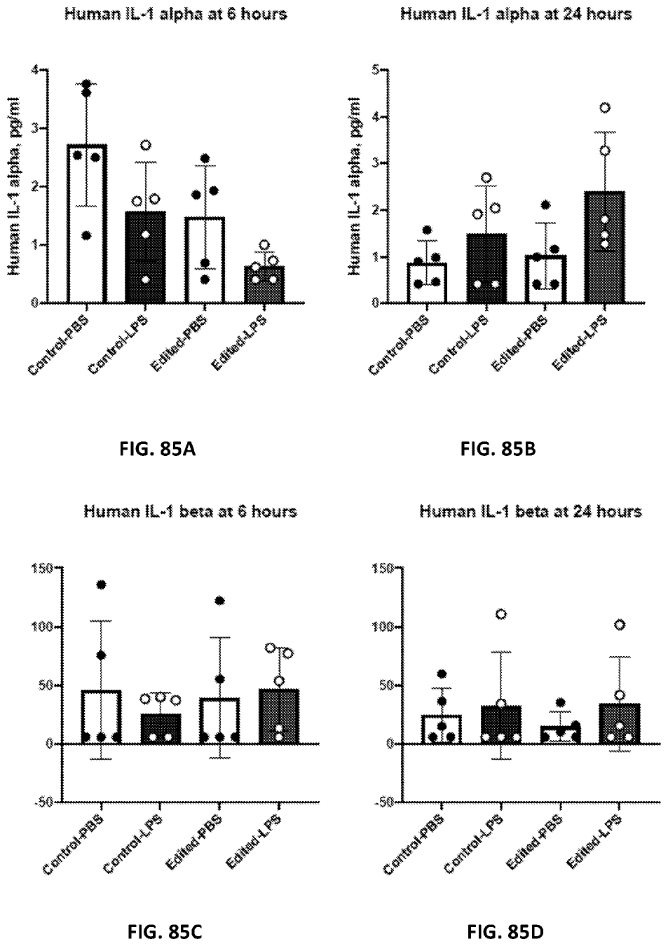

The concentration of IL-1A and IL-1B in the culture medium was measured with species-specific commercial assays, following the manufacturer's instructions. Prior to measurement, frozen media were thawed and then centrifuged (1,500 g for 2 mins) in order to remove cellular debris. Aliquots of medium were measured in duplicate and the concentration of IL-1 determined from a standard curve of recombinant human or canine IL-1A or IL-1B, as appropriate. The results of IL-1 alpha release in canine cells are shown in FIGS. 84A (6 hours) and 84B (24 hours). The results of IL-1 beta release in canine cells are shown in FIGS. 84C (6 hours) and 84D (24 hours). The results of IL-1 alpha release in human cells are shown in FIGS. 85A (6 hours) and 85B (24 hours). The results of IL-1 beta release in human cells are shown in FIGS. 85C (6 hours) and 85D (24 hours).

Taken together, these results demonstrated the functional impact on editing either human or canine cells.

### Example 3: Impact of Cas9 Mutant with Enhanced Specificity

The analysis of gene editing specificity reported in Example 8 was repeated using an enhanced Specificity CRISPR associated protein 9. The eSpCas9 includes three specificity enhancing mutations: K848A, K1003A, and R1060A, as described in Slaymaker et al., Science, 351:84-88 (2016). Th eSpCas9 was expressed in E. coli and purified to homogeneity. The construct has a molecular weight of 161 kDa and contains N-terminal Flag-tags and a C-terminal hexa-His-tag. The sequence of the eSpCas9 is:

(SEQ ID NO: 2733)
MDYKDHDGDYKDHDIDYKDDDDKMAPKKKRKVGIHGVPAADKKYSIGLD
IGTNSVGWAVITDEYKVPSKKFKVLGNTDRHSIKKNLIGALLFDSGETA
EATRLKRTARRRYTRRKNRICYLQEIFSNEMAKVDDSFFHRLEESFLVE
EDKKHERHPIFGNIVDEVAYHEKYPTIYHLRKKLVDSTDKADLRLIYLA
LAHMIKFRGHFLIEGDLNPDNSDVDKLFIQLVQTYNQLFEENPINASGV
DAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSD
ILRVNTEITKAPLSASMIKRYDEHHQDLTLLKALVRQQLPEKYKEIFFD
QSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYY
VGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIV
DLLFKTNRKVTVKQLKEDYFKKIECFDSVEISGVEDRFNASLGTYHDLL
KIIKDKDFLDNEENEDILEDIVLTLTLFEDREMIEERLKTYAHLFDDKV
MKQLKRRRYTGWGRLSRKLINGIRDKQSGKTILDFLKSDGFANRNFMQL
IHDDSLTFKEDIQKAQVSGQGDSLHEHIANLAGSPAIKKGILQTVKVVD
ELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVP
QSFLADDSIDNKVLTRSDKNRGKSDNVPSEEVVKKMKNYWRQLLNAKLI
TQRKFDNLTKAERGGLSELDKAGFIKRQLVETRQITKHVAQILDSRMNT
KYDENDKLIREVKVITLKSKLVSDFRKDFQFYKVREINNYHHAHDAYLN
AVVGTALIKKYPALESEFVYGDYKVYDVRKMIAKSEQEIGKATAKYFFY
SNIMNFFKTEITLANGEIRKAPLIETNGETGEIVWDKGRDFATVRKVLS
MPQVNIVKKTEVQTGGFSKESILPKRNSDKLIARKKDWDPKKYGGFDSP
TVAYSVLVVAKVEKGKSKKLKSVKELLGITIMERSSFEKNPIDFLEAKG
YKEVKKDLIIKLPKYSLFELENGRKRMLASAGELQKGNELALPSKYVNF
LYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDR
KRYTSTKEVLDATLIHQSITGLYETRIDLSQLGGDKRPAATKKAGQAKK
KKAAALEHHHHHH.

Briefly, the same sgRNAs used in Example 1, and shown in FIG. 83, were complexed with the eSpCas9. Single guide RNAs (sgRNAs), fusing the selected crRNA guide sequences to a scaffold sequence were then synthesised (Synthego) with scaffold modifications designed to increase their stability and decrease their cellular immunogenicity. Primers for genotyping were designed to be at least 200 bp from the target site and generate PCR amplicons <1.5 kb and synthesized (Merck).

The following quantities were used for single electroporation-based transfection using the 4D-nucleofector (Lonza, Catalog AAF-1002B and AAF-1002X) and nucleocuvette strips. 80 pmol synthesised sgRNA were pre-complexed with eSpCas9 nuclease at room temperature for at least 10 min. 300-400K dissociated cells were washed with PBS before resuspending them in 20 μl supplemented P3 nucleofection solution and adding the Cas9 RNP complex. These cells were then transferred into a nucleocuvette well and electroporated using the pulse code ER-100. Directly after electroporation, the nucleocuvette was placed into the 37° C./5% CO2 incubator for 10 min for the cells to recover from the electrical voltage. Afterwards, 80 μl growth medium was added to the nucleocuvette well and cells transferred into 6-well dishes with prewarmed growth medium.

Between two- and eleven-days post-electroporation, genomic DNA was extracted from 50-200K cells using DNeasy Blood & Tissue kit (Qiagen, Catalog 69506). Single gRNA target (and off-target) regions were amplified by PCR.

PCR products were size-verified by gel electrophoresis, purified using QIAquick PCR purification kit (Qiagen, Catalog 28106) and submitted for Sanger sequencing at Source BioScience. Sanger traces (ab1) were deconvoluted using ICE version 1.2 (found online at the URL github.com/synthego-open/ice) to infer CRISPR edits. In addition, machine-learning predictions of gene editing using the selected probes was generated using inDelphi. In addition, the predicted off-target sites were analysed through direct sequencing to verify whether gRNA facilitates off-target editing.

As compared to the Cas9 editing reported in Example 1, use of eSpCas9 reduces off-target editing without losing on-target activity. For example, the off-target editing by sgRNA #242 (targeting cIL1B) of three loci, having 2, 3, and 3 mismatches, respectively, were evaluated by amplifying and then sequencing the loci reported in Table 3. As shown in Table 3, the first off-target loci experienced no editing in the experiment described in Example 8, and was not tested here. The second off-target loci experienced almost complete off-target editing (98-99%) in the experiment described in Example 8, but experienced no editing when eSpCas9 was used. The third off-target loci experienced some editing (0-25%) in the experiment described in Example 8, but again experienced no editing when eSpCas9 was used. Further, as shown in Table 2, the “enhanced on-target score,” corresponding to editing using eSpCas9 as described in this example, for each sgRNA tested was as high, if not higher, than the “on-target score,” corresponding to the editing described in Example 1.

TABLE 2

| | | | On-target | | | | |
|---|---|---|---|---|---|---|---|
| | | | metrics | | | | |
| | Target | | On-target | Precision | Frameshift | Enhanced on-target | Off-target metrics |
| # | Exon # | Strand | score* | score** | %*** | score**** | Off-target score\hat{ } |
| sg235 | 3 | − | 69.5 | 0.57 | 93.7 | 72.4 | 67 |
| sg236 | 4 | − | 53.9 | 0.65 | 93.9 | 66.7 | 76.9 |

Human IL1B (IL1B-201; GRCh38)

| | | | | | | | |
|---|---|---|---|---|---|---|---|
| sg237 | 3 | + | 60.9 | 0.57 | 80 | 64.7 | 66.7 |
| sg238 | 4 | − | 58.3 | 0.6 | 86.3 | 65.7 | 49.3 |
| sg248 | 5 | + | 61.7 | 0.65 | 89.9 | 69.6 | 86.5 |
| sg249 | 5 | + | 68.2 | 0.52 | 75.3 | 65.9 | 93.8 |
| sg250 | 5 | − | 64.4 | 0.48 | 83.9 | 65.2 | 91 |

Canine IL1A (IL1A-201; CanFam3.1)

| | | | | | | | |
|---|---|---|---|---|---|---|---|
| sg239 | 3 | + | 49.5 | 0.55 | 78.3 | 58.1 | 57.7 |
| sg240 | 4 | + | 43.8 | 0.61 | 87.2 | 59.0 | 41.8 |
| sg251 | 5 | + | 72.1 | 0.49 | 77.6 | 67.7 | 85.1 |
| sg252 | 5 | − | 68.4 | 0.48 | 90.4 | 68.8 | 66.6 |

TABLE 3

| | | | | | | | | | CRISPR |
|---|---|---|---|---|---|---|---|---|---|
| | | | | | | | | CRISPR | Edits with |
| | | | | | | | | Edits | enhanced |
| | | | | Chrom- | | Posi- | Mis- | with | specificity |
| Sequence | PAM | Score | Gene | osome | Strand | tion | matches | Cas9 | Cas9 |
| ACTCTTGTTACAG | GGG | 100 | ENSCAFG00 | chr17 | 1 | 37022194 | 0 | | |
| AGCTGGT | | | 000007249 | | | | | | |
| ACTTTTGTTTCAG | CAG | 6.16161972 | | chr33 | −1 | 20234937 | 2 | 0% | |
| AGCTGGT | | | | | | | | | |
| CCTCATGCTACAG | GGG | 2.76564774 | | chr1 | −1 | 47541563 | 3 | 98-99% | 0% |
| AGCTGGT | | | | | | | | | |
| GTGCTTGTTACAG | GGG | 2.32143742 | | chr26 | −1 | 32323843 | 3 | 0-25% | 0% |
| AGCTGGT | | | | | | | | | |

### Example 4: Selection of gRNAs Targeting IL1A and IL1B in Humans and Canines

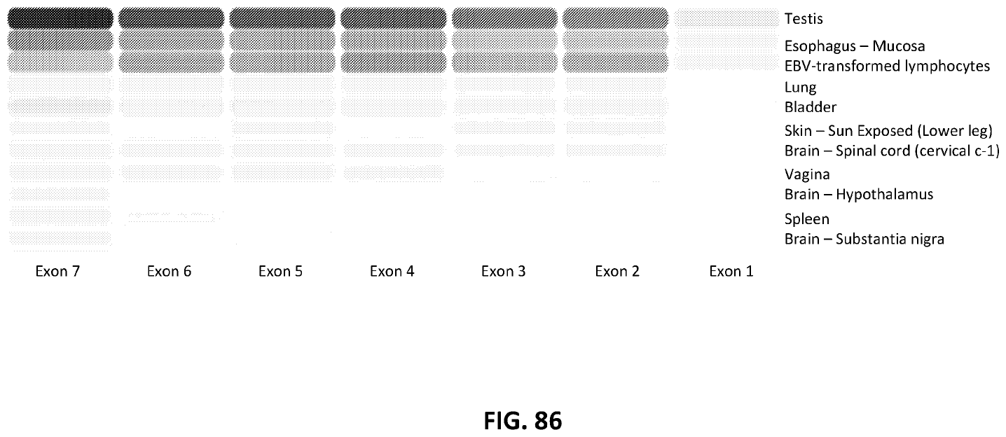

Given that the ultimate goal of CRISPR target design is fabrication of nucleotide sequences that will hybridize to genomic DNA sequences resulting in the most robust knockout of a targeted gene as part of the CRISPR/Cas system, the process begins with assessment of splicing at the target loci. Human IL1A exhibits almost exclusively canonical splicing (i.e., no major variants) across various tissue types with the mature mRNA including exons 2-7, making each of these a potential CRISPR gRNA target (FIG. 86). Additional functional analysis (see Michlits, et al. (2020). Nature Methods, 17(7), 708-716) of the hIL-1a gene demonstrated that all functional domains cluster within Exons 5-7. In order to avoid a truncation that retains residual post-editing functionality, hIL-1a CRISPR targets were limited to those upstream of the functional domain cluster (exons 2-4). In so doing, a resultant frameshift or premature stop codon (i.e., missense mutation) at the editing site will impact all functional domains.

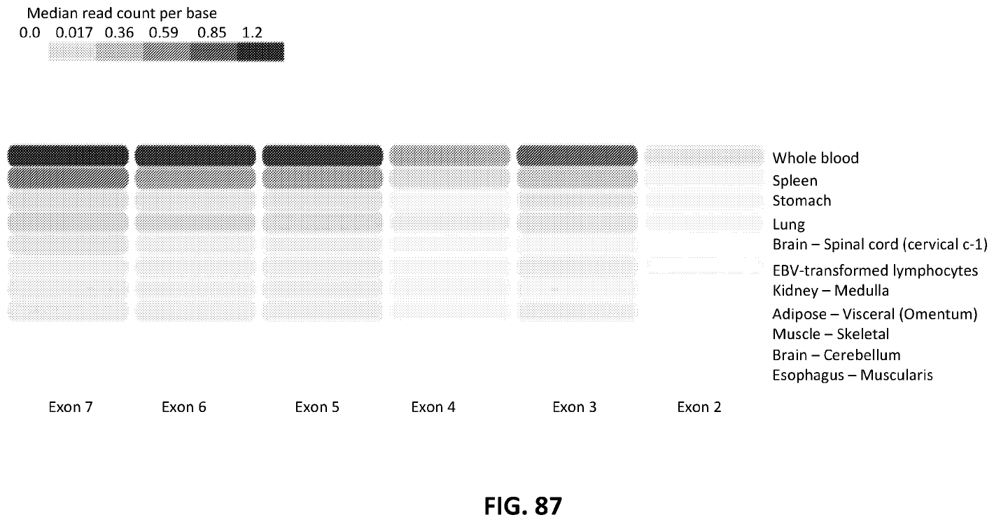

Similar analysis of hIL1B found a stronger overall expression pattern and more variation in splicing as compared to hIL1A (FIG. 87). However, as no tissue exhibited a variant omitting exons 2-7, each of these remained viable CRISPR targets. Application of the same functional analysis tools for hIL-1b found that functional domains cluster in exons 5-7, leaving exons 2-4 as viable CRISPR targets.

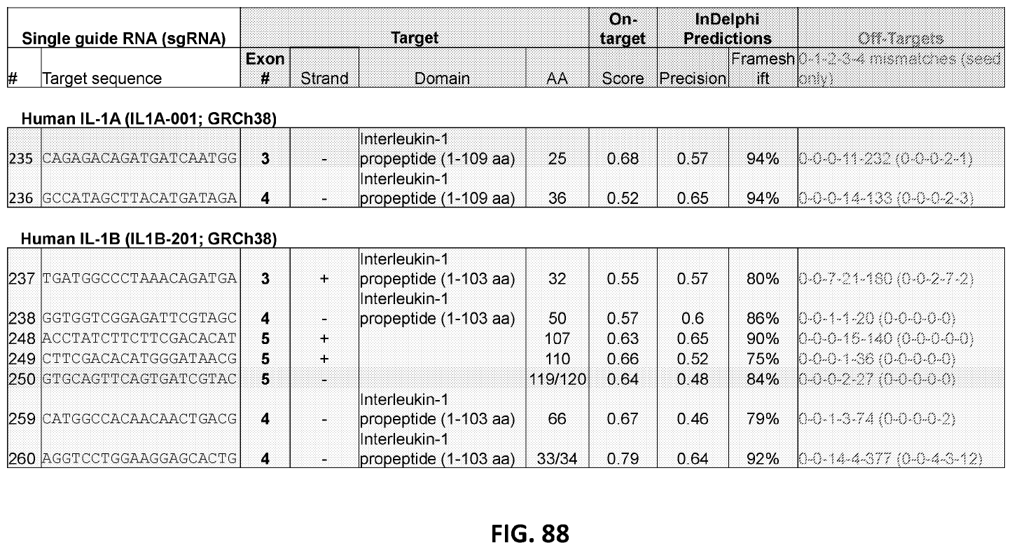

Having established the human gene targets, emphasis then shifted to gRNA targeting domain design. Generated CRISPR targeting domains were first tested in silico through at least, four separate algorithms, yielding scores assessing cutting activity (On-Target score; see Doench et al. (2016). Nature Biotechnology, 34(2), 184-191), reproducibility of the particular mutation via double-strand break repair mechanisms (Precision score; inDelphi), likelihood of creating a frameshift mutation (Frameshift score; inDelphi), and specificity of gRNA binding (Off-Target score; CRISPOR) (FIG. 88). Cutoffs were set for On-Target score at >0.30 and for Off-Target scores of 0 for 0 or 1 mismatch (first two columns).

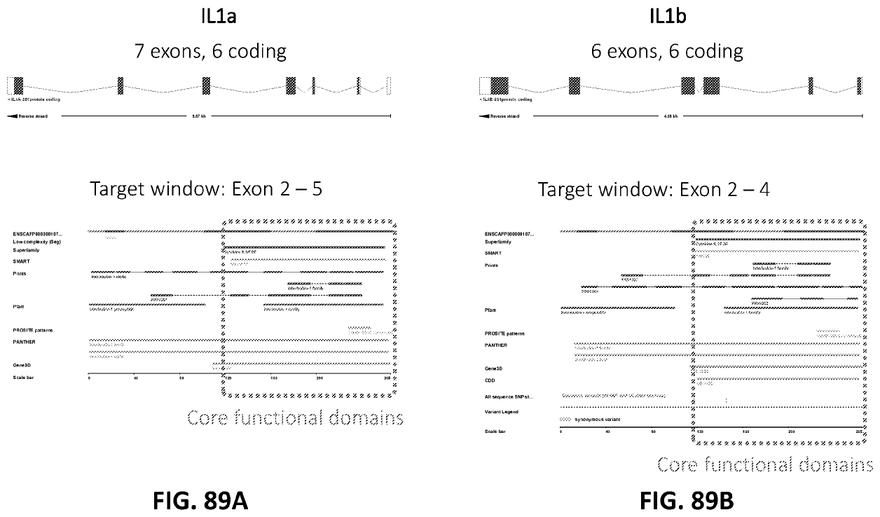

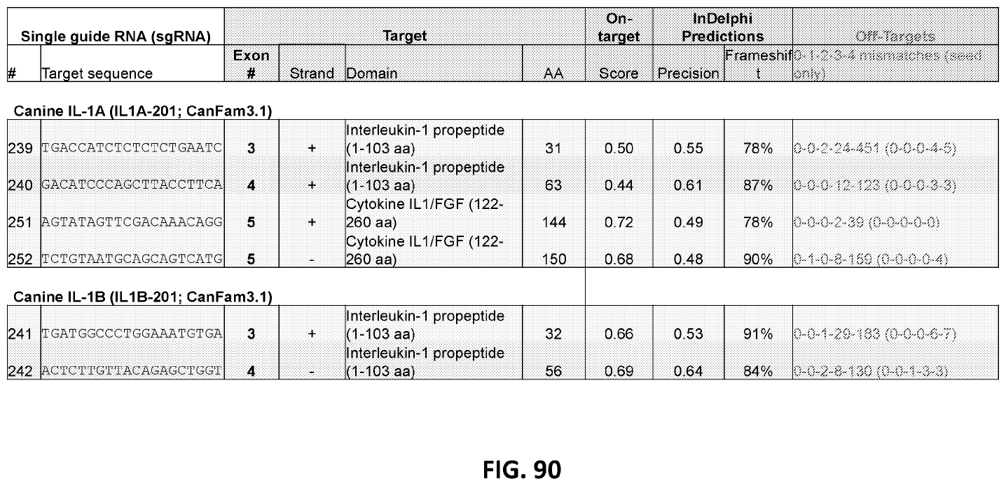

The same design process was then repeated for the orthologous canine gene targets. Splicing and functional analyses of canine IL-1a (cIL-1a) shows 6 exons (exons 2-7) incorporated in the mature mRNA, of which exons 6 and 7 contain functional domains (FIG. 89A). This leaves exons 2-5 as viable CRISPR targets. The mature mRNA of cIL1B contains exons 2-7, with the core function domains clustered in exons 5-7 (FIG. 89B). As such, exons 2-4 are potentially viable CRISPR targets. Generated candidate CRISPR targeting domains were then analyzed in silico; those exceeding the minimum cutoff scores are shown in FIG. 90.

### Example 5: Characterizing the Ablation of IL1A and IL1B in Primary Human and Canine Cells

Algorithm-validated gRNA targeting domains were then tested in primary cells. Briefly, plasmid DNA encoding a sgRNA with the selected targeting domain was introduced into the primary cells with an encoded Cas9 plasmid via electroporation. Pooled cell populations then underwent DNA extraction and sequencing to assess editing efficiency.

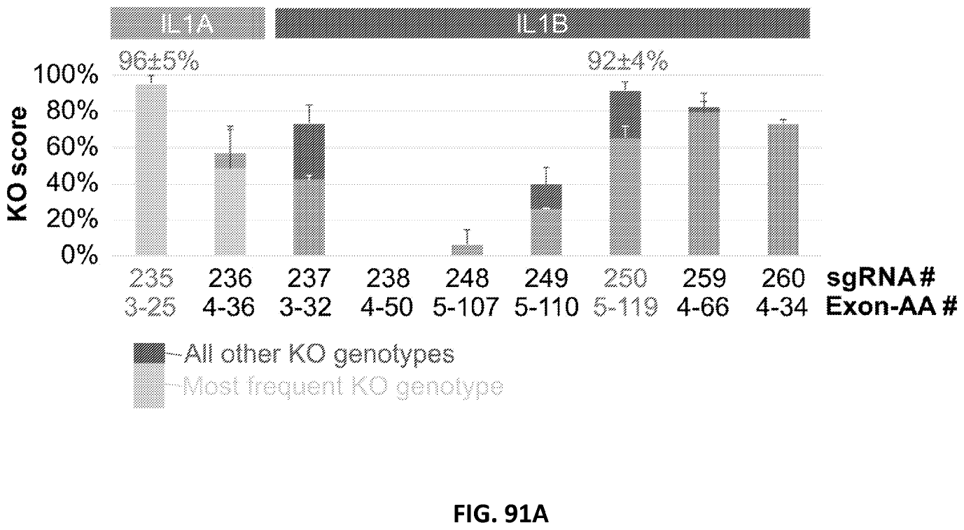

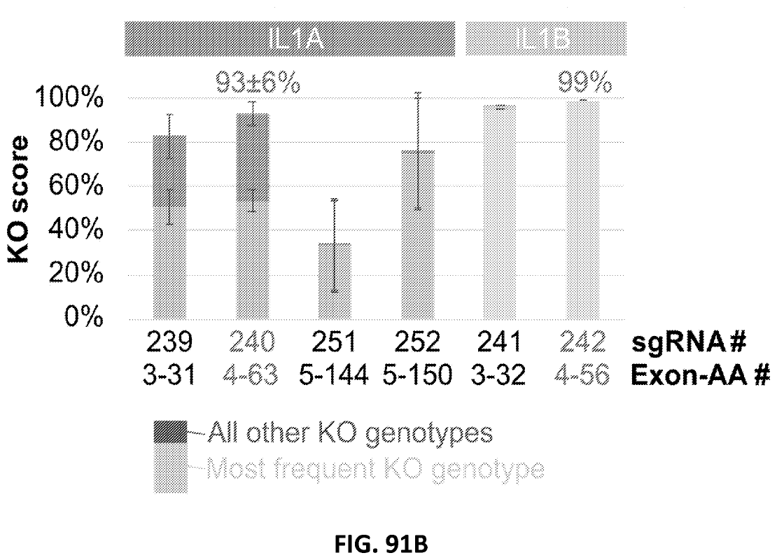

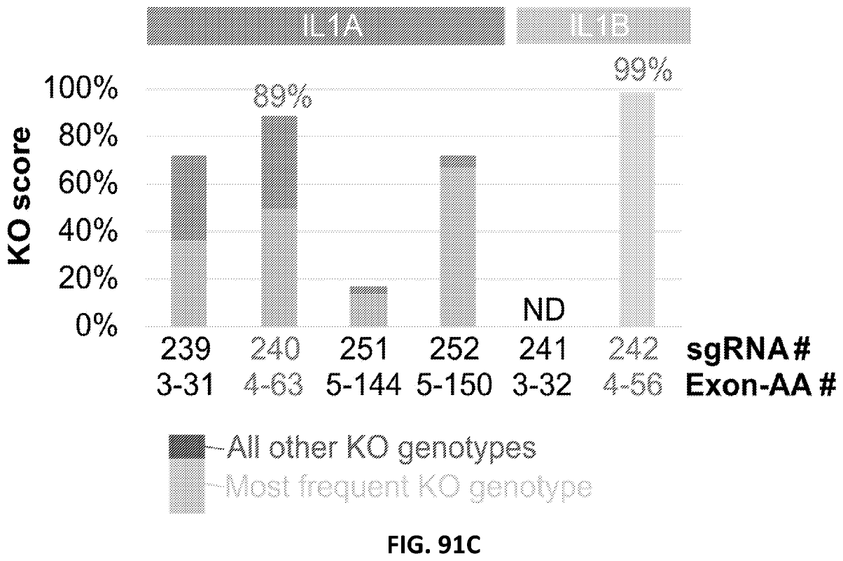

The results, shown in FIG. 91, demonstrate a wide range of editing efficiencies in both human (FIG. 91A) and canine (FIGS. 91B-91C) cells. Indeed, for each gene target, at least one targeting domain demonstrates effective editing (between 89% and 99%) with reproducibility between different cell types (in canine).

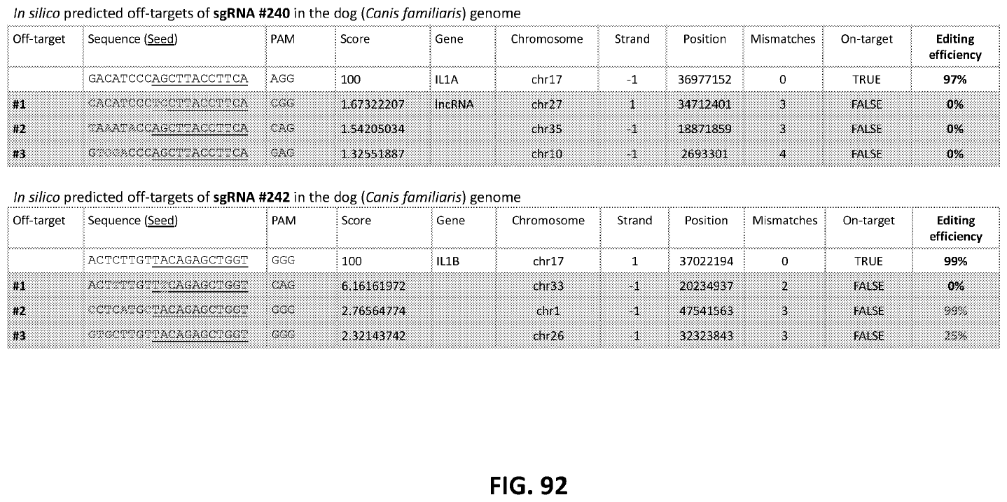

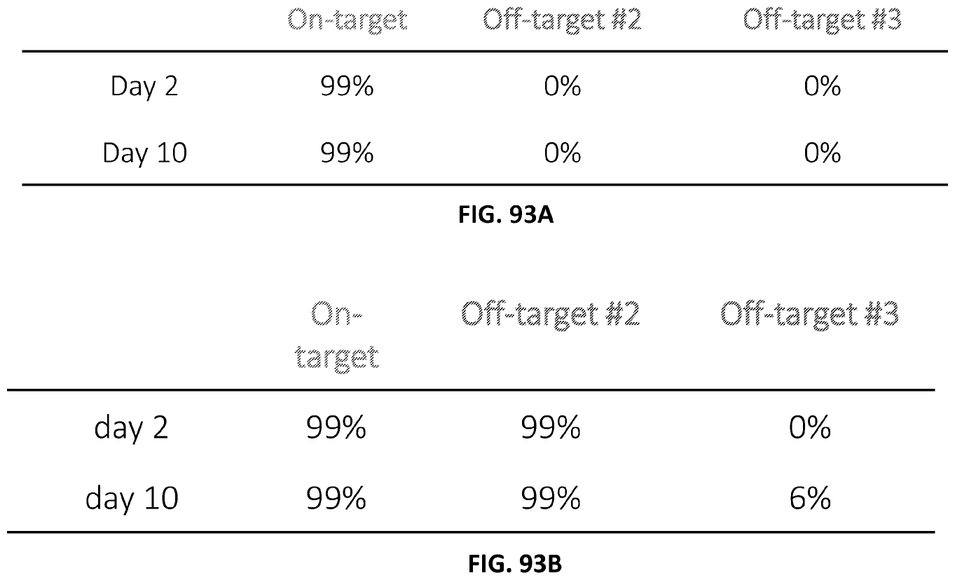

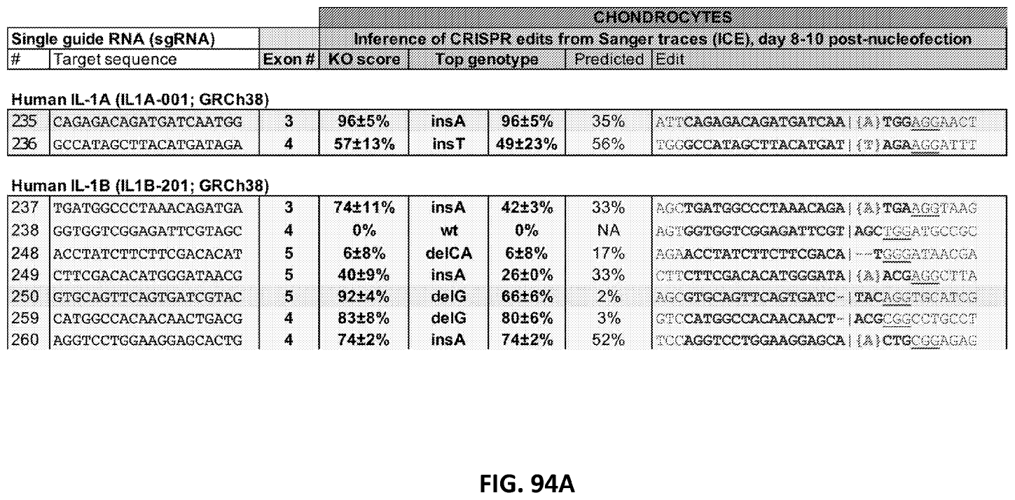

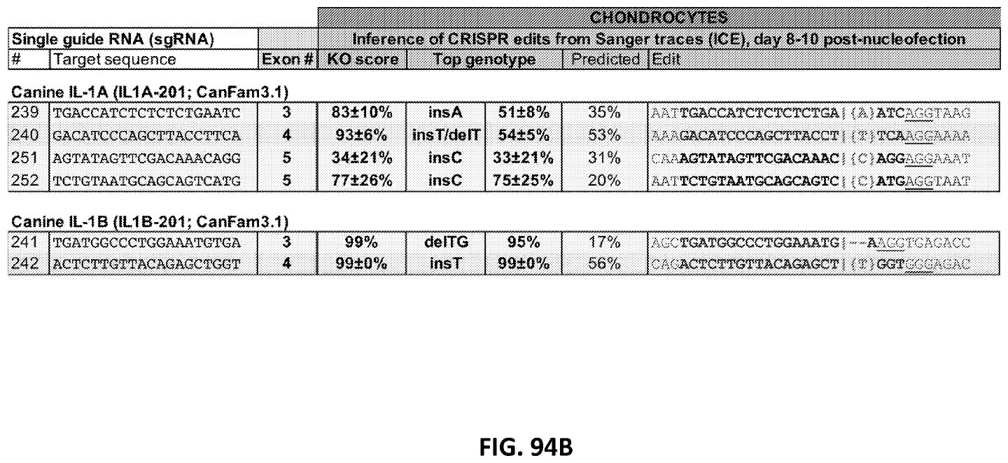

However, in addition to this robust editing of the gene target, sgRNA 242, which targets cIL1B, also exhibited high levels of off-target editing, as anticipated by the in silico analysis (FIG. 92). To rectify these off-target effects, the experiments were repeated with an engineered, enhanced-specificity Cas9 (eSpCas9). This engineered Cas9 completely abrogated the previously-observed off-target effects while still maintain maximal editing efficiency at the target site (FIG. 93). These data demonstrate that the lead candidate sgRNA targeting domain for each gene target can safely (i.e., without off-target effects) and reliably generate genetic knockouts through creation of a primary missense mutation within the targeted locus in human (FIG. 94A) and canine (FIG. 94B) chondrocytes.

Taken together, these data presented a strong profile for ablating gene expression of IL1A and IL1B in primary mammalian cells.

### Example 6: Coadministration of IL1A and IL1B-Targeted sgRNAs in Primary Mammalian Cells

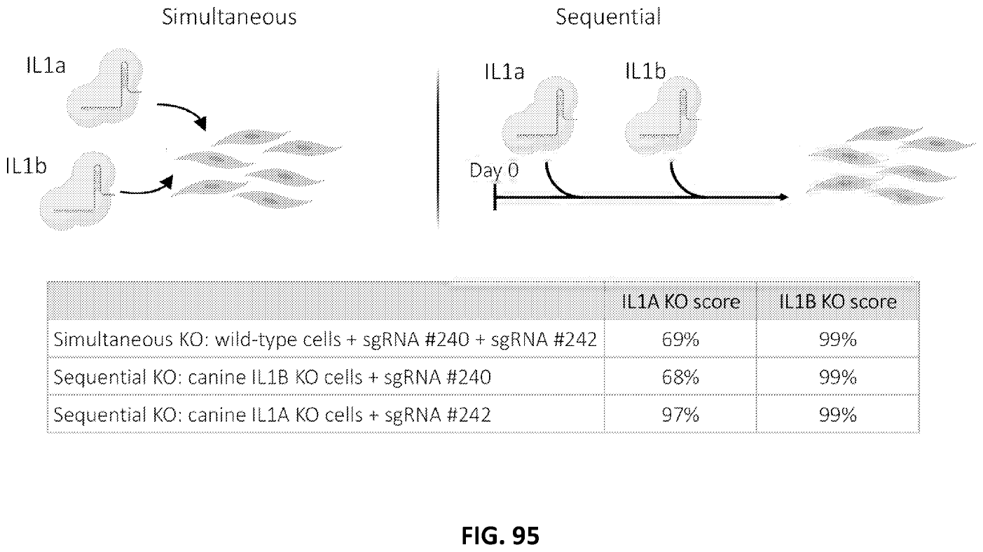

Having observed robust and reproducible efficacy for each individual gRNA targeting domain, the lead candidates were next assessed in the context of co-administration in order to generate double knockouts. To do this, each sgRNA was administered to canine synoviocytes as described above either simultaneously or sequentially (in either order). The results show that sgRNA 242 is highly effective at knocking out cIL1B under all conditions (FIG. 95). Conversely, sgRNA 240, targeting cIL1A, demonstrated optimal efficacy when it edits first with both simultaneous and secondary administration reducing efficacy by roughly 30%. However, such significant reduction likely remains beyond the threshold needed to impact functionality at the organismal level. As such, these results show that more than one sgRNA may be used within a single cell, preferably to silence multiple genes within a single pathway, thereby maximizing functional efficacy.

### Example 7: Applicability to Additional Species

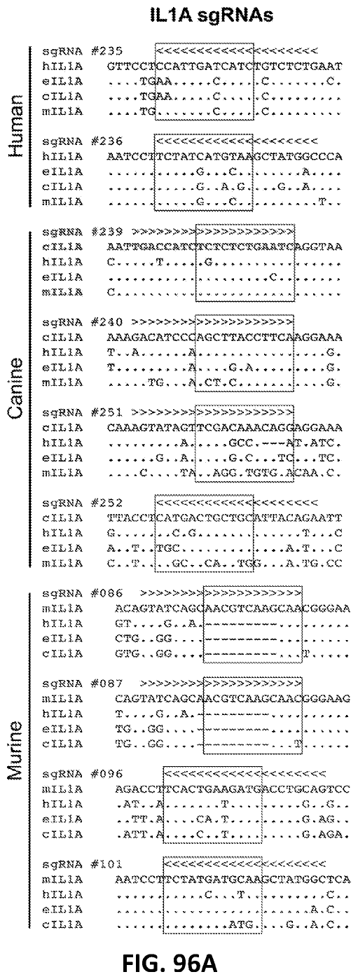

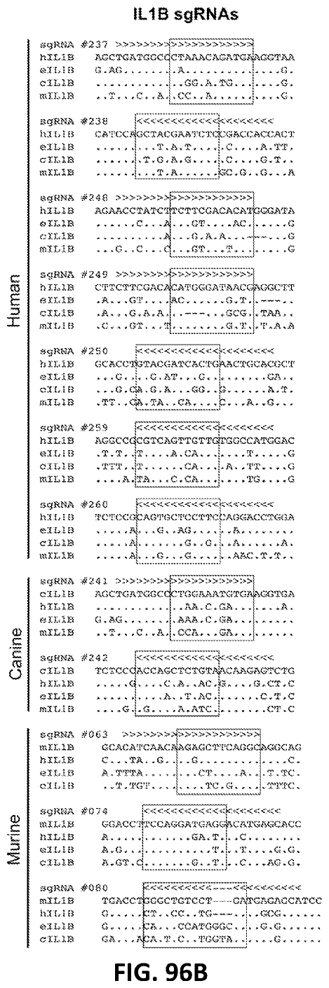

Interleukin 1 is a highly conserved gene in terms of both sequence and function among mammals (see Dinarello, C. A. (1991). Blood 77 (8): 1627-1652). As a result, the gRNA targeting domains that have been generated and characterized for specific species may result in efficient editing of the IL-1 locus of additional species. An alignment to discover conserved IL1A (FIG. 96A) and IL1B (FIG. 96B) gene target positions among humans, horses, dogs, and mice finds relatively few mismatched base pairs across all species at particular target positions. For example, sgRNA 239, targeting cIL1A, is predicted to also edit mouse IL1A. Given the reported flexibility within the CRISPR/Cas system to tolerate imperfect sequence alignments under certain conditions (see generally Zischewski, J., et al. (2017). Biotechnology Advances, 35(1), 95-104), cross-species reactivity for particular targeting domains is wholly anticipated for any number of conserved genes, including, but not limited to, any of those genes listed in Table 4.

TABLE 4
List of Potential pro-inflammatory gene targets.

6-phosphogluconate dehydrogenase (6PGD)
Alcohol dehydrogenase (ADH)
Aldehyde dehydrogenase (ALDH2)
AP-1
B-cell lymphoma-extra large (Bcl-XL)
BCL2 apoptosis regulator (Bcl-2)
Bcl-2-associated X protein (BAX)
Catalase (CAT)
c-Jun N-terminal kinase (JNK)
Coenzyme Q10
CYP2E1
Cytochrome c (Cyt c)
F1Fo-ATP synthase
Ferritin heavy chain (FHC)
Glucose-6-phosphate dehydrogenase (G6PD)
Glutamylcysteine synthetase (GCS)
Glutathione (GSH) synthase
Glutathione Peroxidase 1 (GPX1)
Glutathione Peroxidase 2 (GPX2)
Glutathione Peroxidase 3 (GPX3)
Glutathione Peroxidase 4 (GPX4)
Glutathione Peroxidase 5 (GPX5)
Glutathione Peroxidase 6 (GPX6)
Glutathione Peroxidase 7 (GPX7)
Glutathione Peroxidase 8 (GPX8)
Glutathione reductase (GR)
Glycerol 3-phosphate dehydrogenase
Growth arrest and DNA damage (GADD 45)
Hypoxia-inducible factor 1-alpha (HIF-1α)
Mitogen-activated protein kinase (MAPK)
NADH-ubiquinone oxidoreductase
NADPH oxidase 4 (NOX4)
NADPH oxidase 5 (NOX5)
Nuclear factor kappa-light-chain-enhancer of activated B cells (NF-κB)
Nuclear factor κB (NF-κB) essential modulator (NEMO)
p46Shc (SHC isoform)
p52Shc (SHC isoform)
p53 upregulated modulator of apoptosis (PUMA)
p66Shc (SHC isoform)
Phosphoinositide 3-kinase (PI3-K)
Proline oxidase (PIG6, POX)
Quinone oxidoreductase (PIG3, NQO1)
Sestrin 1 (SESN1)
Sestrin 2 (SESN2)
SHC adaptor protein 1 (SHC1)
Superoxide dismutase 1 (SOD1)
Superoxide dismutase 2 (SOD2)
Superoxide dismutase 3 (SOD3)
TNF alpha induced protein 3 (TNFAIP3)
Tumor protein 53 (p53)
Tumor protein p53 inducible nuclear protein 1 (TP53INP1)
Ubiquinol-cytochrome c oxidoreductase
2B4
ABCA1
ACP5
ADAR-1
ADSS
AIG1
AIM2
APOBEC3
ARRB2
B2M
BCAS3
BMP4
C10orf32
C21orf33
CASP1
CCL5
CD160
Cd53
CDKN2A
CHEK1
CNNM2
CNTNAP2
CSMD1
CTLA-4
CTSB
C-type lectin receptors CLRs
CXCL10
CYP17A1
DDX60
DYNC1I1
FOXO3a
GPC6
GRN
HCK
HECW1
HLA
IFI30
IFI44L
IFI6
IFITM
IFITM1/3
IFITM2
IFITM3
IL-18
IL-1α
IL-1β
interferon-γ
interleukin-12 (IL-12)
IRF
IRF-1
IRF3
IRF7
LAG-3
LIPC
MDA5/IFIH1
MPAK
MYH9
MYO16
MYO5A
NAIP
NF-κB
NLRC4
NLRP3
NOD2
nucleotide oligomerization and binding domain NOD-like receptors
OAS1
OAS2
OASL
parkin gene (PARK2)
PD-1
PLEKHG1
PRKCA
PTBP1
PYCARD
Pyrin-HIN (PYHIN) domain containing receptors (e.g. AIM2)
Any retinoic acid inducible gene-I (RIG-I)-like receptors (RLRs)_{—}
RFC3
RGS1
RIG-I/DDX58
SAMHD1
SF3A1/SF3B1
SFXN2
SLAMF7
SLC41A1
SLC8A1
SLCO3A1
STAT1
Tetherin
TLR5
TLR7
TLR9
Any Toll-like receptors (TLRs)
TREM2
TREX1
TRIM5
TTLL7
TYROBP

### Example 8: LNP Systems

In some embodiments, the following LNP systems are used to formulate pharmaceutical compositions described herein:

TABLE 5
LNP Systems (all values are a molar ratio relative to a 100 total)

| | | | Steroid | |
|---|---|---|---|---|
| | Ionizable | Neutral/Helper | System | PEGylated |
| | Lipid Component | Lipid Component | Component | Lipid Component |
| LNP | (molar ratio or molar | (molar ratio or | (molar ratio or | (molar ratio or |
| System# | ratio range) | molar ratio range) | molar ratio range) | molar ratio range) |
| LNP001 | Dlin-KC2-DMA (42-50) | DSPC (8-10) | (36-46) | (balance to 100) |
| LNP002 | Dlin-KC2-DMA (42-50) | DSPC (10-12) | (36-46) | (balance to 100) |
| LNP003 | Dlin-KC2-DMA (42-50) | DSPC (10 ± 0.5) | (36-46) | (balance to 100) |
| LNP004 | Dlin-KC2-DMA (42-50) | DOPE (8-10) | (36-46) | (balance to 100) |
| LNP005 | Dlin-KC2-DMA (42-50) | DOPE (10-12) | (36-46) | (balance to 100) |
| LNP006 | Dlin-KC2-DMA (42-50) | DOPE (10 ± 0.5) | (36-46) | (balance to 100) |
| LNP007 | Dlin-KC2-DMA (42-50) | DOTMA (8-10) | (36-46) | (balance to 100) |
| LNP008 | Dlin-KC2-DMA (42-50) | DOTMA (10-12) | (36-46) | (balance to 100) |
| LNP009 | Dlin-KC2-DMA (42-50) | DOTMA (10 ± 0.5) | (36-46) | (balance to 100) |
| LNP010 | Dlin-KC2-DMA (42-50) | DPPC (8-10) | (36-46) | (balance to 100) |
| LNP011 | Dlin-KC2-DMA (42-50) | DPPC (10-12) | (36-46) | (balance to 100) |
| LNP012 | Dlin-KC2-DMA (42-50) | DPPC (10 ± 0.5) | (36-46) | (balance to 100) |
| LNP013 | Dlin-KC2-DMA (40-48) | DSPC (8-10) | (36-46) | (balance to 100) |
| LNP014 | Dlin-KC2-DMA (40-48) | DSPC (10-12) | (36-46) | (balance to 100) |
| LNP015 | Dlin-KC2-DMA (40-48) | DSPC (10 ± 0.5) | (36-46) | (balance to 100) |
| LNP016 | Dlin-KC2-DMA (40-48) | DOPE (8-10) | (36-46) | (balance to 100) |
| LNP017 | Dlin-KC2-DMA (40-48) | DOPE (10-12) | (36-46) | (balance to 100) |
| LNP018 | Dlin-KC2-DMA (40-48) | DOPE (10 ± 0.5) | (36-46) | (balance to 100) |
| LNP019 | Dlin-KC2-DMA (40-48) | DOTMA (8-10) | (36-46) | (balance to 100) |
| LNP020 | Dlin-KC2-DMA (40-48) | DOTMA (10-12) | (36-46) | (balance to 100) |
| LNP021 | Dlin-KC2-DMA (40-48) | DOTMA (10 ± 0.5) | (36-46) | (balance to 100) |
| LNP022 | Dlin-KC2-DMA (40-48) | DPPC (8-10) | (36-46) | (balance to 100) |
| LNP023 | Dlin-KC2-DMA (40-48) | DPPC (10-12) | (36-46) | (balance to 100) |
| LNP024 | Dlin-KC2-DMA (40-48) | DPPC (10 ± 0.5) | (36-46) | (balance to 100) |
| LNP025 | Dlin-KC2-DMA (43-45) | DSPC (8-10) | (36-46) | (balance to 100) |
| LNP026 | Dlin-KC2-DMA (43-45) | DSPC (10-12) | (36-46) | (balance to 100) |
| LNP027 | Dlin-KC2-DMA (43-45) | DSPC (10 ± 0.5) | (36-46) | (balance to 100) |
| LNP028 | Dlin-KC2-DMA (43-45) | DOPE (8-10) | (36-46) | (balance to 100) |
| LNP029 | Dlin-KC2-DMA (43-45) | DOPE (10-12) | (36-46) | (balance to 100) |
| LNP030 | Dlin-KC2-DMA (43-45) | DOPE (10 ± 0.5) | (36-46) | (balance to 100) |
| LNP031 | Dlin-KC2-DMA (43-45) | DOTMA (8-10) | (36-46) | (balance to 100) |
| LNP032 | Dlin-KC2-DMA (43-45) | DOTMA (10-12) | (36-46) | (balance to 100) |
| LNP033 | Dlin-KC2-DMA (43-45) | DOTMA (10 ± 0.5) | (36-46) | (balance to 100) |
| LNP034 | Dlin-KC2-DMA (43-45) | DPPC (8-10) | (36-46) | (balance to 100) |
| LNP035 | Dlin-KC2-DMA (43-45) | DPPC (10-12) | (36-46) | (balance to 100) |
| LNP036 | Dlin-KC2-DMA (43-45) | DPPC (10 ± 0.5) | (36-46) | (balance to 100) |
| LNP037 | Dlin-KC2-DMA | DSPC (8-10) | (36-46) | (balance to 100) |
| | (44 ± 0.5) | | | |
| LNP038 | Dlin-KC2-DMA | DSPC (10-12) | (36-46) | (balance to 100) |
| | (44 ± 0.5) | | | |
| LNP039 | Dlin-KC2-DMA | DSPC (10 ± 0.5) | (36-46) | (balance to 100) |
| | (44 ± 0.5) | | | |
| LNP040 | Dlin-KC2-DMA | DOPE (8-10) | (36-46) | (balance to 100) |
| | (44 ± 0.5) | | | |
| LNP041 | Dlin-KC2-DMA | DOPE (10-12) | (36-46) | (balance to 100) |
| | (44 ± 0.5) | | | |
| LNP042 | Dlin-KC2-DMA | DOPE (10 ± 0.5) | (36-46) | (balance to 100) |
| | (44 ± 0.5) | | | |
| LNP043 | Dlin-KC2-DMA | DOTMA (8-10) | (36-46) | (balance to 100) |
| | (44 ± 0.5) | | | |
| LNP044 | Dlin-KC2-DMA | DOTMA (10-12) | (36-46) | (balance to 100) |
| | (44 ± 0.5) | | | |
| LNP045 | Dlin-KC2-DMA | DOTMA (10 ± 0.5) | (36-46) | (balance to 100) |
| | (44 ± 0.5) | | | |
| LNP046 | Dlin-KC2-DMA | DPPC (8-10) | (36-46) | (balance to 100) |
| | (44 ± 0.5) | | | |
| LNP047 | Dlin-KC2-DMA | DPPC (10-12) | (36-46) | (balance to 100) |
| | (44 ± 0.5) | | | |
| LNP048 | Dlin-KC2-DMA | DPPC (10 ± 0.5) | (36-46) | (balance to 100) |
| | (44 ± 0.5) | | | |
| LNP049 | Dlin-MC3-DMA (44-50) | DSPC (8-10) | (36-46) | (balance to 100) |
| LNP050 | Dlin-MC3-DMA (44-50) | DSPC (10-12) | (36-46) | (balance to 100) |
| LNP051 | Dlin-MC3-DMA (44-50) | DSPC (10 ± 0.5) | (36-46) | (balance to 100) |
| LNP052 | Dlin-MC3-DMA (44-50) | DOPE (8-10) | (36-46) | (balance to 100) |
| LNP053 | Dlin-MC3-DMA (44-50) | DOPE (10-12) | (36-46) | (balance to 100) |
| LNP054 | Dlin-MC3-DMA (44-50) | DOPE (10 ± 0.5) | (36-46) | (balance to 100) |
| LNP055 | Dlin-MC3-DMA (44-50) | DOTMA (8-10) | (36-46) | (balance to 100) |
| LNP056 | Dlin-MC3-DMA (44-50) | DOTMA (10-12) | (36-46) | (balance to 100) |
| LNP057 | Dlin-MC3-DMA (44-50) | DOTMA (10 ± 0.5) | (36-46) | (balance to 100) |
| LNP058 | Dlin-MC3-DMA (44-50) | DPPC (8-10) | (36-46) | (balance to 100) |
| LNP059 | Dlin-MC3-DMA (44-50) | DPPC (10-12) | (36-46) | (balance to 100) |
| LNP060 | Dlin-MC3-DMA (44-50) | DPPC (10 ± 0.5) | (36-46) | (balance to 100) |
| LNP061 | Dlin-MC3-DMA (50-54) | DSPC (8-10) | (36-46) | (balance to 100) |
| LNP062 | Dlin-MC3-DMA (50-54) | DSPC (10-12) | (36-46) | (balance to 100) |
| LNP063 | Dlin-MC3-DMA (50-54) | DSPC (10 ± 0.5) | (36-46) | (balance to 100) |
| LNP064 | Dlin-MC3-DMA (50-54) | DOPE (8-10) | (36-46) | (balance to 100) |
| LNP065 | Dlin-MC3-DMA (50-54) | DOPE (10-12) | (36-46) | (balance to 100) |
| LNP066 | Dlin-MC3-DMA (50-54) | DOPE (10 ± 0.5) | (36-46) | (balance to 100) |
| LNP067 | Dlin-MC3-DMA (50-54) | DOTMA (8-10) | (36-46) | (balance to 100) |
| LNP068 | Dlin-MC3-DMA (50-54) | DOTMA (10-12) | (36-46) | (balance to 100) |
| LNP069 | Dlin-MC3-DMA (50-54) | DOTMA (10 ± 0.5) | (36-46) | (balance to 100) |
| LNP070 | Dlin-MC3-DMA (50-54) | DPPC (8-10) | (36-46) | (balance to 100) |
| LNP071 | Dlin-MC3-DMA (50-54) | DPPC (10-12) | (36-46) | (balance to 100) |
| LNP072 | Dlin-MC3-DMA (50-54) | DPPC (10 ± 0.5) | (36-46) | (balance to 100) |
| LNP073 | Dlin-MC3-DMA (49-51) | DSPC (8-10) | (36-46) | (balance to 100) |
| LNP074 | Dlin-MC3-DMA (49-51) | DSPC (10-12) | (36-46) | (balance to 100) |
| LNP075 | Dlin-MC3-DMA (49-51) | DSPC (10 ± 0.5) | (36-46) | (balance to 100) |
| LNP076 | Dlin-MC3-DMA (49-51) | DOPE (8-10) | (36-46) | (balance to 100) |
| LNP077 | Dlin-MC3-DMA (49-51) | DOPE (10-12) | (36-46) | (balance to 100) |
| LNP078 | Dlin-MC3-DMA (49-51) | DOPE (10 ± 0.5) | (36-46) | (balance to 100) |
| LNP079 | Dlin-MC3-DMA (49-51) | DOTMA (8-10) | (36-46) | (balance to 100) |
| LNP080 | Dlin-MC3-DMA (49-51) | DOTMA (10-12) | (36-46) | (balance to 100) |
| LNP081 | Dlin-MC3-DMA (49-51) | DOTMA (10 ± 0.5) | (36-46) | (balance to 100) |
| LNP082 | Dlin-MC3-DMA (49-51) | DPPC (8-10) | (36-46) | (balance to 100) |
| LNP083 | Dlin-MC3-DMA (49-51) | DPPC (10-12) | (36-46) | (balance to 100) |
| LNP084 | Dlin-MC3-DMA (49-51) | DPPC (10 ± 0.5) | (36-46) | (balance to 100) |
| LNP085 | Dlin-MC3-DMA | DSPC (8-10) | (36-46) | (balance to 100) |
| | (50 ± 0.5) | | | |
| LNP086 | Dlin-MC3-DMA | DSPC (10-12) | (36-46) | (balance to 100) |
| | (50 ± 0.5) | | | |
| LNP087 | Dlin-MC3-DMA | DSPC (10 ± 0.5) | (36-46) | (balance to 100) |
| | (50 ± 0.5) | | | |
| LNP088 | Dlin-MC3-DMA | DOPE (8-10) | (36-46) | (balance to 100) |
| | (50 ± 0.5) | | | |
| LNP089 | Dlin-MC3-DMA | DOPE (10-12) | (36-46) | (balance to 100) |
| | (50 ± 0.5) | | | |
| LNP090 | Dlin-MC3-DMA | DOPE (10 ± 0.5) | (36-46) | (balance to 100) |
| | (50 ± 0.5) | | | |
| LNP091 | Dlin-MC3-DMA | DOTMA (8-10) | (36-46) | (balance to 100) |
| | (50 ± 0.5) | | | |
| LNP092 | Dlin-MC3-DMA | DOTMA (10-12) | (36-46) | (balance to 100) |
| | (50 ± 0.5) | | | |
| LNP093 | Dlin-MC3-DMA | DOTMA (10 ± 0.5) | (36-46) | (balance to 100) |
| | (50 ± 0.5) | | | |
| LNP094 | Dlin-MC3-DMA | DPPC (8-10) | (36-46) | (balance to 100) |
| | (50 ± 0.5) | | | |
| LNP095 | Dlin-MC3-DMA | DPPC (10-12) | (36-46) | (balance to 100) |
| | (50 ± 0.5) | | | |
| LNP096 | Dlin-MC3-DMA | DPPC (10 ± 0.5) | (36-46) | (balance to 100) |
| | (50 ± 0.5) | | | |
| LNP097 | SM-102 (44-50) | DSPC (8-10) | (36-46) | (balance to 100) |
| LNP098 | SM-102 (44-50) | DSPC (10-12) | (36-46) | (balance to 100) |
| LNP099 | SM-102 (44-50) | DSPC (10 ± 0.5) | (36-46) | (balance to 100) |
| LNP100 | SM-102 (44-50) | DOPE (8-10) | (36-46) | (balance to 100) |
| LNP101 | SM-102 (44-50) | DOPE (10-12) | (36-46) | (balance to 100) |
| LNP102 | SM-102 (44-50) | DOPE (10 ± 0.5) | (36-46) | (balance to 100) |
| LNP103 | SM-102 (44-50) | DOTMA (8-10) | (36-46) | (balance to 100) |
| LNP104 | SM-102 (44-50) | DOTMA (10-12) | (36-46) | (balance to 100) |
| LNP105 | SM-102 (44-50) | DOTMA (10 ± 0.5) | (36-46) | (balance to 100) |
| LNP106 | SM-102 (44-50) | DPPC (8-10) | (36-46) | (balance to 100) |
| LNP107 | SM-102 (44-50) | DPPC (10-12) | (36-46) | (balance to 100) |
| LNP108 | SM-102 (44-50) | DPPC (10 ± 0.5) | (36-46) | (balance to 100) |
| LNP109 | SM-102 (50-54) | DSPC (8-10) | (36-46) | (balance to 100) |
| LNP110 | SM-102 (50-54) | DSPC (10-12) | (36-46) | (balance to 100) |
| LNP111 | SM-102 (50-54) | DSPC (10 ± 0.5) | (36-46) | (balance to 100) |
| LNP112 | SM-102 (50-54) | DOPE (8-10) | (36-46) | (balance to 100) |
| LNP113 | SM-102 (50-54) | DOPE (10-12) | (36-46) | (balance to 100) |
| LNP114 | SM-102 (50-54) | DOPE (10 ± 0.5) | (36-46) | (balance to 100) |
| LNP115 | SM-102 (50-54) | DOTMA (8-10) | (36-46) | (balance to 100) |
| LNP116 | SM-102 (50-54) | DOTMA (10-12) | (36-46) | (balance to 100) |
| LNP117 | SM-102 (50-54) | DOTMA (10 ± 0.5) | (36-46) | (balance to 100) |
| LNP118 | SM-102 (50-54) | DPPC (8-10) | (36-46) | (balance to 100) |
| LNP119 | SM-102 (50-54) | DPPC (10-12) | (36-46) | (balance to 100) |
| LNP120 | SM-102 (50-54) | DPPC (10 ± 0.5) | (36-46) | (balance to 100) |
| LNP121 | SM-102 (49-51) | DSPC (8-10) | (36-46) | (balance to 100) |
| LNP122 | SM-102 (49-51) | DSPC (10-12) | (36-46) | (balance to 100) |
| LNP123 | SM-102 (49-51) | DSPC (10 ± 0.5) | (36-46) | (balance to 100) |
| LNP124 | SM-102 (49-51) | DOPE (8-10) | (36-46) | (balance to 100) |
| LNP125 | SM-102 (49-51) | DOPE (10-12) | (36-46) | (balance to 100) |
| LNP126 | SM-102 (49-51) | DOPE (10 ± 0.5) | (36-46) | (balance to 100) |
| LNP127 | SM-102 (49-51) | DOTMA (8-10) | (36-46) | (balance to 100) |
| LNP128 | SM-102 (49-51) | DOTMA (10-12) | (36-46) | (balance to 100) |
| LNP129 | SM-102 (49-51) | DOTMA (10 ± 0.5) | (36-46) | (balance to 100) |
| LNP130 | SM-102 (49-51) | DPPC (8-10) | (36-46) | (balance to 100) |
| LNP131 | SM-102 (49-51) | DPPC (10-12) | (36-46) | (balance to 100) |
| LNP132 | SM-102 (49-51) | DPPC (10 ± 0.5) | (36-46) | (balance to 100) |
| LNP133 | SM-102 (50 ± 0.5) | DSPC (8-10) | (36-46) | (balance to 100) |
| LNP134 | SM-102 (50 ± 0.5) | DSPC (10-12) | (36-46) | (balance to 100) |
| LNP135 | SM-102 (50 ± 0.5) | DSPC (10 ± 0.5) | (36-46) | (balance to 100) |
| LNP136 | SM-102 (50 ± 0.5) | DOPE (8-10) | (36-46) | (balance to 100) |
| LNP137 | SM-102 (50 ± 0.5) | DOPE (10-12) | (36-46) | (balance to 100) |
| LNP138 | SM-102 (50 ± 0.5) | DOPE (10 ± 0.5) | (36-46) | (balance to 100) |
| LNP139 | SM-102 (50 ± 0.5) | DOTMA (8-10) | (36-46) | (balance to 100) |
| LNP140 | SM-102 (50 ± 0.5) | DOTMA (10-12) | (36-46) | (balance to 100) |
| LNP141 | SM-102 (50 ± 0.5) | DOTMA (10 ± 0.5) | (36-46) | (balance to 100) |
| LNP142 | SM-102 (50 ± 0.5) | DPPC (8-10) | (36-46) | (balance to 100) |
| LNP143 | SM-102 (50 ± 0.5) | DPPC (10-12) | (36-46) | (balance to 100) |
| LNP144 | SM-102 (50 ± 0.5) | DPPC (10 ± 0.5) | (36-46) | (balance to 100) |
| LNP145 | ALC-0315 (44-50) | DSPC (8-10) | (36-46) | (balance to 100) |
| LNP146 | ALC-0315 (44-50) | DSPC (10-12) | (36-46) | (balance to 100) |
| LNP147 | ALC-0315 (44-50) | DSPC (10 ± 0.5) | (36-46) | (balance to 100) |
| LNP148 | ALC-0315 (44-50) | DOPE (8-10) | (36-46) | (balance to 100) |
| LNP149 | ALC-0315 (44-50) | DOPE (10-12) | (36-46) | (balance to 100) |
| LNP150 | ALC-0315 (44-50) | DOPE (10 ± 0.5) | (36-46) | (balance to 100) |
| LNP151 | ALC-0315 (44-50) | DOTMA (8-10) | (36-46) | (balance to 100) |
| LNP152 | ALC-0315 (44-50) | DOTMA (10-12) | (36-46) | (balance to 100) |
| LNP153 | ALC-0315 (44-50) | DOTMA (10 ± 0.5) | (36-46) | (balance to 100) |
| LNP154 | ALC-0315 (44-50) | DPPC (8-10) | (36-46) | (balance to 100) |
| LNP155 | ALC-0315 (44-50) | DPPC (10-12) | (36-46) | (balance to 100) |
| LNP156 | ALC-0315 (44-50) | DPPC (10 ± 0.5) | (36-46) | (balance to 100) |
| LNP157 | ALC-0315 (50-54) | DSPC (8-10) | (36-46) | (balance to 100) |
| LNP158 | ALC-0315 (50-54) | DSPC (10-12) | (36-46) | (balance to 100) |
| LNP159 | ALC-0315 (50-54) | DSPC (10 ± 0.5) | (36-46) | (balance to 100) |
| LNP160 | ALC-0315 (50-54) | DOPE (8-10) | (36-46) | (balance to 100) |
| LNP161 | ALC-0315 (50-54) | DOPE (10-12) | (36-46) | (balance to 100) |
| LNP162 | ALC-0315 (50-54) | DOPE (10 ± 0.5) | (36-46) | (balance to 100) |
| LNP163 | ALC-0315 (50-54) | DOTMA (8-10) | (36-46) | (balance to 100) |
| LNP164 | ALC-0315 (50-54) | DOTMA (10-12) | (36-46) | (balance to 100) |
| LNP165 | ALC-0315 (50-54) | DOTMA (10 ± 0.5) | (36-46) | (balance to 100) |
| LNP166 | ALC-0315 (50-54) | DPPC (8-10) | (36-46) | (balance to 100) |
| LNP167 | ALC-0315 (50-54) | DPPC (10-12) | (36-46) | (balance to 100) |
| LNP168 | ALC-0315 (50-54) | DPPC (10 ± 0.5) | (36-46) | (balance to 100) |
| LNP169 | ALC-0315 (49-51) | DSPC (8-10) | (36-46) | (balance to 100) |
| LNP170 | ALC-0315 (49-51) | DSPC (10-12) | (36-46) | (balance to 100) |
| LNP171 | ALC-0315 (49-51) | DSPC (10 ± 0.5) | (36-46) | (balance to 100) |
| LNP172 | ALC-0315 (49-51) | DOPE (8-10) | (36-46) | (balance to 100) |
| LNP173 | ALC-0315 (49-51) | DOPE (10-12) | (36-46) | (balance to 100) |
| LNP174 | ALC-0315 (49-51) | DOPE (10 ± 0.5) | (36-46) | (balance to 100) |
| LNP175 | ALC-0315 (49-51) | DOTMA (8-10) | (36-46) | (balance to 100) |
| LNP176 | ALC-0315 (49-51) | DOTMA (10-12) | (36-46) | (balance to 100) |
| LNP177 | ALC-0315 (49-51) | DOTMA (10 ± 0.5) | (36-46) | (balance to 100) |
| LNP178 | ALC-0315 (49-51) | DPPC (8-10) | (36-46) | (balance to 100) |
| LNP179 | ALC-0315 (49-51) | DPPC (10-12) | (36-46) | (balance to 100) |
| LNP180 | ALC-0315 (49-51) | DPPC (10 ± 0.5) | (36-46) | (balance to 100) |
| LNP181 | ALC-0315 (50 ± 0.5) | DSPC (8-10) | (36-46) | (balance to 100) |
| LNP182 | ALC-0315 (50 ± 0.5) | DSPC (10-12) | (36-46) | (balance to 100) |
| LNP183 | ALC-0315 (50 ± 0.5) | DSPC (10 ± 0.5) | (36-46) | (balance to 100) |
| LNP184 | ALC-0315 (50 ± 0.5) | DOPE (8-10) | (36-46) | (balance to 100) |
| LNP185 | ALC-0315 (50 ± 0.5) | DOPE (10-12) | (36-46) | (balance to 100) |
| LNP186 | ALC-0315 (50 ± 0.5) | DOPE (10 ± 0.5) | (36-46) | (balance to 100) |
| LNP187 | ALC-0315 (50 ± 0.5) | DOTMA (8-10) | (36-46) | (balance to 100) |
| LNP188 | ALC-0315 (50 ± 0.5) | DOTMA (10-12) | (36-46) | (balance to 100) |
| LNP189 | ALC-0315 (50 ± 0.5) | DOTMA (10 ± 0.5) | (36-46) | (balance to 100) |
| LNP190 | ALC-0315 (50 ± 0.5) | DPPC (8-10) | (36-46) | (balance to 100) |
| LNP191 | ALC-0315 (50 ± 0.5) | DPPC (10-12) | (36-46) | (balance to 100) |
| LNP192 | ALC-0315 (50 ± 0.5) | DPPC (10 ± 0.5) | (36-46) | (balance to 100) |
| LNP193 | LP01 (42-45) | DSPC (8-10) | (36-46) | (balance to 100) |
| LNP194 | LP01 (42-45) | DSPC (10-12) | (36-46) | (balance to 100) |
| LNP195 | LP01 (42-45) | DSPC (10 ± 0.5) | (36-46) | (balance to 100) |
| LNP196 | LP01 (42-45) | DOPE (8-10) | (36-46) | (balance to 100) |
| LNP197 | LP01 (42-45) | DOPE (10-12) | (36-46) | (balance to 100) |
| LNP198 | LP01 (42-45) | DOPE (10 ± 0.5) | (36-46) | (balance to 100) |
| LNP199 | LP01 (42-45) | DOTMA (8-10) | (36-46) | (balance to 100) |
| LNP200 | LP01 (42-45) | DOTMA (10-12) | (36-46) | (balance to 100) |
| LNP201 | LP01 (42-45) | DOTMA (10 ± 0.5) | (36-46) | (balance to 100) |
| LNP202 | LP01 (42-45) | DPPC (8-10) | (36-46) | (balance to 100) |
| LNP203 | LP01 (42-45) | DPPC (10-12) | (36-46) | (balance to 100) |
| LNP204 | LP01 (42-45) | DPPC (10 ± 0.5) | (36-46) | (balance to 100) |
| LNP205 | LP01 (45-50) | DSPC (8-10) | (36-46) | (balance to 100) |
| LNP206 | LP01 (45-50) | DSPC (10-12) | (36-46) | (balance to 100) |
| LNP207 | LP01 (45-50) | DSPC (10 ± 0.5) | (36-46) | (balance to 100) |
| LNP208 | LP01 (45-50) | DOPE (8-10) | (36-46) | (balance to 100) |
| LNP209 | LP01 (45-50) | DOPE (10-12) | (36-46) | (balance to 100) |
| LNP210 | LP01 (45-50) | DOPE (10 ± 0.5) | (36-46) | (balance to 100) |
| LNP211 | LP01 (45-50) | DOTMA (8-10) | (36-46) | (balance to 100) |
| LNP212 | LP01 (45-50) | DOTMA (10-12) | (36-46) | (balance to 100) |
| LNP213 | LP01 (45-50) | DOTMA (10 ± 0.5) | (36-46) | (balance to 100) |
| LNP214 | LP01 (45-50) | DPPC (8-10) | (36-46) | (balance to 100) |
| LNP215 | LP01 (45-50) | DPPC (10-12) | (36-46) | (balance to 100) |
| LNP216 | LP01 (45-50) | DPPC (10 ± 0.5) | (36-46) | (balance to 100) |
| LNP217 | LP01 (44-46) | DSPC (8-10) | (36-46) | (balance to 100) |
| LNP218 | LP01 (44-46) | DSPC (10-12) | (36-46) | (balance to 100) |
| LNP219 | LP01 (44-46) | DSPC (10 ± 0.5) | (36-46) | (balance to 100) |
| LNP220 | LP01 (44-46) | DOPE (8-10) | (36-46) | (balance to 100) |
| LNP221 | LP01 (44-46) | DOPE (10-12) | (36-46) | (balance to 100) |
| LNP222 | LP01 (44-46) | DOPE (10 ± 0.5) | (36-46) | (balance to 100) |
| LNP223 | LP01 (44-46) | DOTMA (8-10) | (36-46) | (balance to 100) |
| LNP224 | LP01 (44-46) | DOTMA (10-12) | (36-46) | (balance to 100) |
| LNP225 | LP01 (44-46) | DOTMA (10 ± 0.5) | (36-46) | (balance to 100) |
| LNP226 | LP01 (44-46) | DPPC (8-10) | (36-46) | (balance to 100) |
| LNP227 | LP01 (44-46) | DPPC (10-12) | (36-46) | (balance to 100) |
| LNP228 | LP01 (44-46) | DPPC (10 ± 0.5) | (36-46) | (balance to 100) |
| LNP229 | LP01 (45 ± 0.5) | DSPC (8-10) | (36-46) | (balance to 100) |
| LNP230 | LP01 (45 ± 0.5) | DSPC (10-12) | (36-46) | (balance to 100) |
| LNP231 | LP01 (45 ± 0.5) | DSPC (10 ± 0.5) | (36-46) | (balance to 100) |
| LNP232 | LP01 (45 ± 0.5) | DOPE (8-10) | (36-46) | (balance to 100) |
| LNP233 | LP01 (45 ± 0.5) | DOPE (10-12) | (36-46) | (balance to 100) |
| LNP234 | LP01 (45 ± 0.5) | DOPE (10 ± 0.5) | (36-46) | (balance to 100) |
| LNP235 | LP01 (45 ± 0.5) | DOTMA (8-10) | (36-46) | (balance to 100) |
| LNP236 | LP01 (45 ± 0.5) | DOTMA (10-12) | (36-46) | (balance to 100) |
| LNP237 | LP01 (45 ± 0.5) | DOTMA (10 ± 0.5) | (36-46) | (balance to 100) |
| LNP238 | LP01 (45 ± 0.5) | DPPC (8-10) | (36-46) | (balance to 100) |
| LNP239 | LP01 (45 ± 0.5) | DPPC (10-12) | (36-46) | (balance to 100) |
| LNP240 | LP01 (45 ± 0.5) | DPPC (10 ± 0.5) | (36-46) | (balance to 100) |

In some embodiments of any one LNP system LNP001 to LNP240, the LNP system comprises the steroid component in a molar ratio from about 36 to about 46, or from about 38 to about 42. In some embodiments of any one LNP system LNP001 to LNP240, the LNP system comprises the steroid component in a molar ratio of about 36±0.5, about 37±0.5, about 38±0.5, about 39±0.5, about 40±0.5, about 41±0.5, about 42±0.5, about 43±0.5, about 44±0.5, about 45±0.5, or about ±0.5. In some embodiments, the LNP system comprises the steroid component in a molar ratio of about 37, about 38, about 39, about 40, about 41, or about 42. In some embodiments of any one LNP system LNP001 to LNP240, the steroid component comprises cholesterol. In some embodiments of any one LNP system LNP001 to LNP240, the steroid component comprises cholesterol and one or more additional steroids, wherein cholesterol is about 60% (w/w) or more, about 65% (w/w) or more, about 70% (w/w) or more, about 75% (w/w) or more, or about 80% (w/w) or more of the steroid component. In some embodiments, the steroid component comprises dexamethasone. In some embodiments, the steroid component comprises a modified cholesterol, e.g., a hydroxy- or alkyl modification. In some embodiments, the steroid component comprises any other steroid disclosed herein. In some embodiments, the LNP system comprises the PEGylated Lipid Component in a molar ratio from about 1.5 to about 2.5. In some embodiments, the PEGylated Lipid Component comprises a PEG2000 lipid. In some embodiments, the PEGylated Lipid Component comprises one or more of DMG-PEG and/or DMG-C-PEG. In some embodiments, the PEGylated Lipid Component comprises one or more of DMG-PEG2000, DMG-C-PEG2000, DSG-PEG2000, C14-PEG2000, C16-PEG2000, and/or C18-PEG2000.

### Example 9: Screening of LNP Formulations

LNPs described herein typically include a Biodegradable Ionizable Lipid, a Helper phospholipid, Cholesterol (with the potentially addition of dexamethasone or other steroids), and a PEG2000 component. Molar Lipid Ratio described herein are ratio of ionizable lipid:helper lipid:cholesterol [:Dexamethasone]:PEG.

Modifying molar lipid ratios: Without wishing to be bound by any particular theory, when altering the molar lipid ratio of ionizable lipids, the amount of cholesterol or helper lipid would either be supplemented or decreased. Typically, ionizable lipids are kept at a set ratio, while helper lipids, cholesterol and PEG content are varied. If you were to alter the amount of PEG, you would subsequently adjust the amount of primarily cholesterol and sometimes the helper lipid. If altering the helper lipid, the amount of cholesterol in combination with this is adjusted. Dexamethasone, or another similar steroid in the LNP structure directly replaces some of the cholesterol due to similar chemical structures/function in the LNP.

Typical ionizable lipids are: Dlin-KC2-DMA: molar ratios between 42-50, e.g., and without limitation, 44; Dlin-MC3-DMA: molar ratios between 44-50, e.g., and without limitation, 50; SM-102 (Lipid H): molar ratios between 44-50, e.g., and without limitation, 50; ALC-0315: molar ratios between 44-50, e.g., and without limitation, 50; LP01 (LP000001): molar ratios between 42-50, e.g., and without limitation, 45. Helper Lipids can have molar ratios between 9-11, e.g., and without limitation, 10. Helper lipids include DSPC, DOPE, DOTMA, and DPPC. PEG2000 lipids have typical molar ratios between about 1.5 and about 2.5. These include, without limitation, DMG-PEG, DMG-C-PEG, DMG-PEG2000, DMG-C-PEG2000, DSG-PEG2000, and C14,16,18-PEG2000. Cholesterol molar lipid ratios are typically between about 36 and about 46. Dexamethasone or similar steroids supplement a portion of the molar lipid ratio of cholesterol, for example, and without limitation, a ratio of cholesterol:dexamethasone (C:D) of 9:1. Molar lipid ratio ranges of Cholesterol can be, without limitation, between about 8 and about 10 and Dexamethasone, without limitation, between about 0.1 and about 2. Without wishing to be bound by any particular theory, modifications or replacements for cholesterol can be considered, for example, and without limitation, hydroxy- or alkyl modification (e.g., to improve mRNA delivery) or substitution with potentially therapeutic moieties such as anti-inflammatory steroids. In some embodiments, Molar N/P ratios cab be between 1-8. Without wishing to be bound by any particular, it is believed that to reduce inflammatory response of LNPs in vivo, the ideal range is 1-5 for N/P, however, N/P ratios of 6-8 are tested.

In some embodiments, LNP formulations described herein include LP01:DSPC:Cholesterol:DMG-PEG2000, LP01:DSPC:Cholesterol:Dexamethasone:DMG-PEG2000, MC3:DSPC:Cholesterol:DMG-PEG2000, MC3:DSPC:Cholesterol:Dexamethasone:DMG-PEG2000, MC3:DSPC:Cholesterol:DMG-C-PEG2000, MC3:DSPC:Cholesterol:Dexamethasone:DMG-C-PEG2000, SM-102:DSPC:Cholesterol:DMG-PEG2000, SM-102:DSPC:Cholesterol:Dexamethasone:DMG-PEG2000, ALC-0315:DSPC:Cholesterol:DMG-PEG2000, ALC-0315:DSPC:Cholesterol:Dexamethasone:DMG-PEG2000. In some embodiemnts, molar ratios of formulations described herein include 50:10:38.5:1.5; 45:9:44:2.

Biophysical Assays: LNPs efficiency to encapsulate nucleic acids, meet sizing criteria and are homogeneity, stability after freeze-thaw cycles. Payloads encapsulated: GFP mRNA, Luciferase mRNA, and our CRISPR/cas9 therapeutic (sgRNA and cas9 mRNA).

TABLE 6
LNPs of various formulations created and screened.

| | | | RNA |
|---|---|---|---|
| ID | LNP Composition | Ratio | Payload |
| LNP 02 | MC3:DSPC:Cholesterol:D | 50:10:38.5:1.5 | OCB02::cas9 |
| | MG-PEG2000 | | mRNA |
| LNP 03 | MC3:DSPC:Cholesterol:De | 50:10:34.65: | OCB02::cas9 |
| | xamethasone:DMG- | 3.85:1.5 | mRNA |
| | PEG2000 | | |
| LNP 04 | MC3:DSPC:Cholesterol:D | 50:10:38.5:1.5 | OCB02::cas9 |
| | MG-C-PEG2000 | | mRNA |
| LNP 05 | KC2:DPPC:Cholesterol:C1 | 47.4:10.1: | Luciferase |
| | 6-C-PEG2000 | 39.5:4 | mRNA |
| LNP 07 | MC3:DPSC:Cholesterol:D | 50:10:38.5:1.5 | Luciferase |
| | MG-PEG2000 | | mRNA |

### Additional Notes on LNP Components:

Ionizable lipid (titratable charge): Contains a tertiary amine, making this positively charged at an acidic pH, neutrally charged at a physiological pH. Become protonated after endosomal uptake into the cytosol. See below for specifications on different types of ionizable lipids.

Helper phospholipid: anionic endosomal phospholipids that interact with protonated ionizable lipids to form cone shaped ion pairs that enhance cell membrane fusion and disruption, endosomal escape, and cargo release into cell cytosol.

Cholesterol: maintains nanoparticle membrane integrity, assists encapsulation of nucleic acids, and enhances circulation by reducing surface bound proteins.

Dexamethasone or similar steroids: provide an anti-inflammatory component to the LNPs, which decrease immunogenicity and increase transfection rates, particularly in vivo.

PEGylated lipid (PEG2000): improves circulation half-life, reduces aggregation of LNPs, and reduces interactions with serum proteins such as opsonins, enhancing stability in vivo. Additionally assists particle stability, particle sizing, and biodistribution.

### Ionizable Lipids:

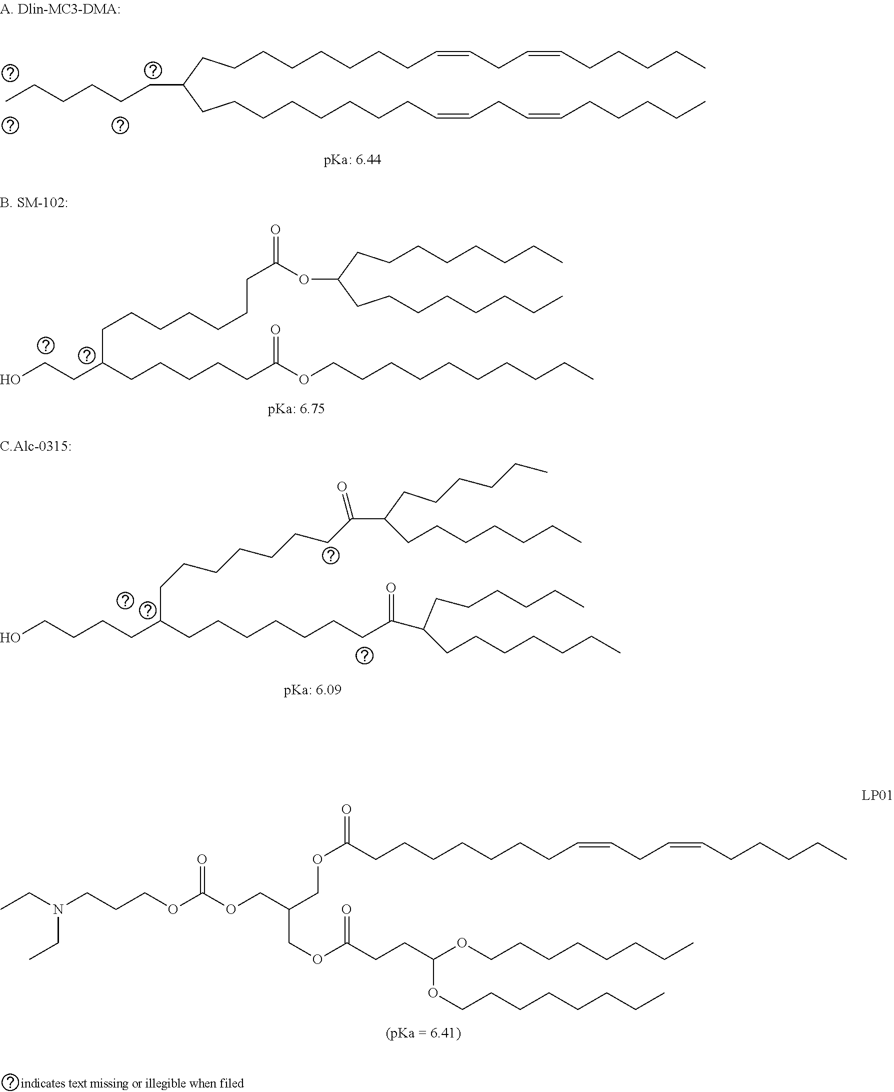

Cationic lipids: such as DODMA, DOTMA, and DOTAP have a quaternary amine group, which leaves them permanently positively charged. Use of this alone was in the past proven to have poor circulation and increased toxicity in vivo. Led to the development of ionizable lipids.

Ionizable lipids are characterised by a replacing quaternary amine to be tertiary, which enables them to be pH dependent, i.e., is neutrally charged at physiological pH and becomes positively charged (protonated) at acidic pH.

This increases circulation half-life and reduces toxicity in vivo.

pKa of ionizable lipid drives performance in vivo. pKas between 6-6.7 have been shown to be optimal for delivery of RNA therapeutics. However, the relative pKa of combination of all components in LNP influences LNP transfection—tertiary amine, quaternary amino and hydroxyl group from ionizable lipid, helper lipids and cholesterol all alter relative pKa due to proximity between headgroups. This subsequently affects overall surface charge.

PH dependency enables efficient encapsulation of RNAs in acidic buffer as well as assists RNA release once uptaken by cells.

Further, these lipid pairs form an inverted hexagonal HII phase, which assists membrane disruption, endosomal escape, and subsequent payload release into cytosol of cells.

Albertson et al., 2022, Lipid packing theory showing relationship between amphipathic compounds and geometry once self-assembled. Proposed mechanism by which ionizable lipids mediate endosomal disruption (also known as molecular shape hypothesis). https://www.ncbi.nlm.nih.gov/pmc/articles/PMC9250827/

MC3, ALC-0135, SM-102 all have tertiary amines (making them ionizable), no stereo centres, and ester linkers. ALC-0135 and SM-102 have been improved upon MC3 to incorporate additional ester bonds (to assist with biodegradability and reduce bioaccumulation).

Unsaturation of linear tails increases delivery efficiency and fluidity in ionizable lipids, i.e., this enables bilayer lipids to form a non-bilayer phase that will increase membrane disruption and payload release. Additional research explored branching the tails of ionizable lipids, which further increased potency when delivering mRNA therapeutics due to an increase in protonation of ionizable lipids at endosomal pH and cross-section of lipid tails. This modification also led to an increase in cone-shape structure, which facilitates membrane disruption, endosomal escape, and payload release into the cytosol (Albertson et al., 2022).

LP01 incorporates increase in ester bonds as well as branching of tails, which has been shown to be an efficient and safe delivery system for in-vivo gene editing in animal models. LP01 has less liver bioaccumulation and fewer safety risks.

### REFERENCES

Hald Albertsen, C, Kulkami J A., Witzigmann D, Lind M, Petersson K and Simonsen J B. The role of lipid components in lipid nanoparticles for vaccines and gene therapy. Advanced Drug Delivery Reviews 2022; 188:114416.
Zhang H, Han X, Alameh M, Shepherd S J, Padilla M S, Xue L, et al. Rational design of anti-inflammatory lipid nanoparticles for mRNA delivery. J Biomed Mater Res A. 2022; 110(5):1101-8.
Finn J D, Smith A R, Patel M C, Shaw L, Youniss M R, Heteren J van, et al. A Single Administration of CRISPR/Cas9 Lipid Nanoparticles Achieves Robust and Persistent In Vivo Genome Editing. Cell Reports. 2018; 22(9):2227-35.
Han X, Zhang H., Butowska K, Swingle K L, Alameh A-G, Weissman A and Mitchell M J. An ionizable lipid toolbox for RNA delivery. Nat Commun 12, 7233 (2021). https://doi.org/10.1038/s41467-021-27493-0

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

The examples set forth above are provided to give those of ordinary skill in the art a complete disclosure and description of how to make and use the embodiments of the compositions, systems and methods of the disclosure, and are not intended to limit the scope of what the inventors regard as their invention. Modifications of the above-described modes for carrying out the embodiments of the disclosure that are obvious to persons of skill in the art are intended to be within the scope of the following claims. All patents and publications mentioned in the specification are indicative of the levels of skill of those skilled in the art to which the disclosure pertains.

All headings and section designations are used for clarity and reference purposes only and are not to be considered limiting in any way. For example, those of skill in the art will appreciate the usefulness of combining various aspects from different headings and sections as appropriate according to the spirit and scope of the disclosure described herein.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

While some embodiments have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure.

Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein can be employed in practicing the disclosure. It is therefore contemplated that the disclosure shall also cover any such alternatives, modifications, variations or equivalents. It is intended that the following claims define the scope of the disclosure and that methods and structures within the scope of these claims and their equivalents be covered thereby.

What is claimed is:

1. A pharmaceutical composition for treating a disorder, the composition comprising a plurality of lipid nanoparticles (LNPs) encapsulating:
(i) an RNA-guided nuclease or a nucleic acid encoding an RNA-guided nuclease; and
(ii) at least one guide RNA or a nucleic acid encoding at least one guide RNA targeting a gene selected from FGF2, CCN2, NGF, NTF3, NTF4, BDNF, FGFR1, NGFR, NTRK1, NTRK2, ADAM17, ADAMTS1, ADAMTS5, MMP1, MMP2, MMP3, MMP7, MMP8, MMP10, MMP12, MMP13, TIMP1, TIMP3 CXCL1, CXCL2, CXCL3, CXCL5, CXCL6, CXCL8, CCL2, CCL3, CCL5, CCL7, CCL20, IL1A, IL1B, IL4, IL6, IL10, IL13, IL17A, IL18, TNF, CXCR1, CXCR2, CCR7, TNFRSF1A, TNFRSF1B, IL1R1, IL1RAP, IL4R, IL6R, IL6ST IL10RA, IL10RB, IL13RA1, IL13RA2, IL17RA, IL18R1, IL18RAP, SCN1A, SCN2A, SCN3A, SCN4A, SCN5A, SCN8A, SCN9A, SCN10A, SCN11A, TAC1, TAC3, TACR1, TACR2, TACR3, ATP1A1, CALCA, CALCB, CALCRL, RAMP1, ADM, CRCP, YAP1, MRGPRX2, TGFB, TGFBR1, and TGFBR2.

2. The pharmaceutical composition of claim 1, wherein the at least one guide RNA comprises a crRNA sequence selected from any one of SEQ ID NOs: 1-2731.

3. The pharmaceutical composition of claim 1 or 2, wherein the disorder is a musculoskeletal disorder.

4. The pharmaceutical composition of claim 3, wherein the musculoskeletal disorder is selected from the group consisting of Rheumatoid Arthritis, Gout, Osteoarthritis, Osteoporosis, Intervertebral disc disease (IVDD), Psoriatic arthritis (PsA), Arthritis, Polymyositis, Proliferative synovitis, Malignant bone neoplasm, Sarcoid Myopathy, Cortex Bone Disorders, Idiopathic Scoliosis, Tendinopathy, Myofibrillar Myopathy, Enthesis-Related Arthritis, Ankylosing spondylitis, Degenerative, polyarthritis, Arthropathy, Osteitis Deformans, Prolapsed Lumbar Disc, Polymyositis Ossificans, Idiopathic Polymyositis, Luft Disease, Adult-onset Still's Disease, Osteoarthrosis Deformans, and Bachet's Disease.

5. The pharmaceutical composition of claim 3, wherein the musculoskeletal disorder is selected from the group consisting of Rheumatoid arthritis, Idiopathic osteoporosis, Post-menopausal osteoporosis, Paget's disease, Osteoarthritis, Juvenile idiopathic arthritis (JIA), Still's disease, Ankylosing spondylitis, Polymyalgia rheumatica, Arthritis, Secondary malignant neoplasm of bone, Type II, Mucolipidosis, Sjogren's Syndrome, Psoriatic arthritis, Rheumatism, Castleman's disease, Degenerative Polyarthritis, Arthropathy, Bone neoplasm, Osteoporosis, Massive osteolysis, Bone fracture healing, Systemic sclerosis, Systemic Juvenile Idiopathic, Arthritis, and Synovitis.

6. The pharmaceutical composition of claim 3, wherein the musculoskeletal disorder is selected from the group consisting of Arthritis, Infectious Intermittent joint effusion, Ankylosing spondylitis, Arthritis, Osteoarthritis, Spondylarthritis, Plantar fasciitis, Degenerative polyarthritis, Hemophilic arthropathy, Inflammatory myopathy with abundant macrophages, Polymyositis, Tendinosis, Malignant Bone Neoplasm, Osteoporosis, Psoriatic arthritis, Rheumatism, Rheumatoid arthritis, Adult Still's disease, Juvenile arthritis, Early rheumatoid arthritis, Palindromic rheumatism, Gout, Infectious Arthritis, Myotonic dystrophy, Dermatomyositis, Hemophilic arthropathy, Osteopenia, Sjogren's syndrome, Juvenile Idiopathic Arthritis, Myasthenia gravis, Osteolysis, Inflammation, Degenerative polyarthritis, Osteitis deformans, Pigmented villonodular synovitis, or Hyperphosphatasemia with bone disease.

7. The pharmaceutical composition of claim 3, wherein the musculoskeletal disorder is selected from the group consisting of Loeys-Dietz Syndrome, Osteoarthrosis, Marfan Syndrome, Aortic aneurysm (familial thoracic 3), and a Craniofacial abnormality.

8. The pharmaceutical composition of claim 1 or 2, wherein the disorder is a neoplasia.

9. The pharmaceutical composition of claim 8, wherein the neoplasia is selected from the group consisting of Osteosarcoma, Colon Cancer, Metastasis (General), Lung Cancer, Multiple Myeloma. Breast Cancer, Solid Tumors, Lymphoma, Pancreatic Cancer, Stomach Cancer, Epithelial Ovarian Cancer, Mammary Neoplasms, Oropharyngeal Carcinoma, Renal Cell Carcinoma, Chondrosarcoma, Esophageal Neoplasms, B-Cell Lymphoma, Cutaneous Lymphoma T-Cell, Leukemia, Thyroid Carcinoma, Skin carcinogenesis, Cholectoral Cancer, Glioma, Liver Cancer, Melanoma, Neuroblastoma, Polycystic Ovary Syndrome, Glioblastoma, Prostate Cancer, Cervical Cancer, Ovarian Cancer, Bladder Cancer, Squamous cell carcinoma, Kaposi Sarcoma, Oral Cavity Cancer, Leiomyosarcoma, Malignant Peripheral Nerve Sheath Tumor, and Ewing's Sarcoma.

10. The pharmaceutical composition of claim 8, wherein the neoplasia is selected from the group consisting of Multiple myeloma, Lung Cancer, Stomach Cancer, Breast Cancer, Kidney Cancer, Neoplasm Metastasis, Colorectal Neoplasms, Ovarian cancer, Osteosarcoma, Cholangiocarcinoma, Leukemia, Prostate cancer, Plasmacytoma, Pancreatic cancer, Cervical cancer, Lymphoma, Liver neoplasms, Neuroblastoma, Melanoma, Mastocytosis, Endometrial cancer, Bladder Cancer, Squamous cell carcinoma, Gallbladder carcinoma, Adenocarcinoma, Thyroid Cancer, prostate neoplasms, stomach cancer, liver carcinoma, pituitary neoplasms, hepatoblastoma, multiple myeloma, hepatocellular adenoma, breast carcinoma, carcinogenesis, leukemia, colon carcinoma, melanoma, metastasis (general), lung cancer, pancreatic cancer, Kaposi sarcoma, medulloblastoma, carcinoma of bladder, squamous cell carcinoma, mast cell neoplasm, glioma, mastocytoma, brain neoplasms, ovarian neoplasms, bone neoplasm, rhabdomyosarcoma, solid tumors, metastatic kidney cancer, and metastatic renal cell carcinoma

11. The pharmaceutical composition of claim 8, wherein the neoplasia is selected from the group consisting of a Neoplasm, Glioblastoma, Astrocytoma, Adenocarcinoma, Osteosarcoma, Squamous cell carcinoma, Metastasis, Ameloblastoma, Cholangiocarcinoma, Choriocarcinoma, Ovarian cancer, Prostate cancer, Lung Cancer, Larynx Cancer, Breast Cancer, Lip and Oral Cavity Carcinoma, Squamous intraepithelial lesion, Neuroblastoma, Non-Hodgkin lymphomas, Malignant Neoplasms, Skin Neoplasms, Neuroblastoma, Neoplasm Metastasis, Colorectal Cancer, Fibrosarcoma, Myeloid Leukemia, Myelofibrosis, Hodgkin Lymphomas, Non-Small Cell Lung Carcinoma, Cervical Cancer, Liver cancer, Extrapulmonary small cell carcinoma, Basal cell carcinoma, Kidney cancer, Pancreatic carcinoma, Mesothelioma, Gastric cancer, Polycystic Ovary Syndrome, Chordoma, Cholangiocarcinoma, Malignant ascites, Nasopharyngeal carcinoma, Head and Neck Carcinoma, Cancer metastasis, Prostate carcinoma, Fibrosarcoma, Liver carcinoma, Carcinoma of bladder, T-Cell Lymphoblastic Leukemia, Bladder Neoplasm, melanoma, Leukemia, acute myeloid leukemia, Hepatocellular carcinoma, Colorectal cancer, a Lymphoma, Mycosis fungoides, and Sezary syndrome.

12. The pharmaceutical composition of claim 8, wherein the neoplasia is selected from the group consisting of Pancreatic cancer, Multiple self-healing squamous epithelioma (Ferguson-Smith disease), Pancreatic cancer, Gastrointestinal Stromal Tumors (GIST), Hereditary Nonpolyposis Colorectal Cancer (Lynch Syndrome), Metastasis, Colorectal Carcinoma, Bone neoplasms, Anaplastic carcinoma, Spindle-Cell carcinoma, Malignant Bone Neoplasm, Lung neoplasms, and Malignant brain neoplasm.

13. The pharmaceutical composition of claim 1 or 2, wherein the disorder is a neurological disorder.

14. The pharmaceutical composition of claim 13, wherein the neurological disorder is selected from the group consisting of Acute Thrombotic Stroke, Epilepsy, Multiple Sclerosis, and Alzheimer's Disease.

15. The pharmaceutical composition of claim 1 or 2, wherein the disorder is a cardiac disorder.

16. The pharmaceutical composition of claim 15, wherein the cardiac disorder is selected from the group consisting of Acute Myocardial Infarction, refractory heart failure, arrhythmias, pericarditis, myocarditis, sepsis-induced cardiomyopathy, Acute Decompensated Heart Failure, Refractory Idiopathic Pericarditis, Atherosclerosis, coronary artery disease, myocardial infarction, and cardiac remodeling.

17. The pharmaceutical composition of claim 15, wherein the cardiac disorder is atherosclerosis or abdominal aortic aneurism.

18. The pharmaceutical composition of claim 1 or 2, wherein the disorder is an inflammatory disorder.

19. The pharmaceutical composition of claim 18, wherein the inflammatory disorder is selected from the group consisting of Autoinflammatory Disease (AID), Cryopyrin Associated Periodic syndrome (CAPS), Familial Mediterranean Fever (FMF), TNF-Receptor Associated Periodic Syndrome (TRAPS), Hyper-IgD Syndrome (HIDS), Systemic Lupus Erythematosus (SLE), and Fibrosis.

20. The pharmaceutical composition of claim 18, wherein the inflammatory disorder is selected from the group consisting of a cytokine storm, acute local inflammation, inflammatory bowel disease, Crohn's disease, sepsis, Experimental sepsis, and Castleman's disease

21. The pharmaceutical composition of claim 1 or 2, wherein the disorder is a digestive disorder.

22. The pharmaceutical composition of claim 21, wherein the digestive disorder is selected from the group consisting of Inflammatory Bowel Disease (IBD), Gastroesophageal reflux disease (GERD), and Non-HP-associated Peptic Ulcer Disease.

23. The pharmaceutical composition of claim 1 or 2, wherein the disorder is a respiratory disorder.

24. The pharmaceutical composition of claim 23, wherein the respiratory disorder is Asthma or Pulmonary Fibrosis.

25. The pharmaceutical composition of claim 1 or 2, wherein the disorder is a renal, metabolic, or ophthalmic disorder.

26. The pharmaceutical composition of claim 25, wherein the renal, metabolic, or ophthalmic disorder is selected from the group consisting of Chronic Kidney Disease, Type 2 Diabetes, an Eye Disease, Uveitis, Scleritis, Sjogren asthenia, and dry eye.

27. The pharmaceutical composition of claim 1 or 2, wherein the disorder is an autoimmune disorder.

28. The pharmaceutical composition of claim 27, wherein the autoimmune disorder is Systemic Lupus Erythematosus.

29. The pharmaceutical composition of claim 1 or 2, wherein the disorder is acute synovitis, chronic synovitis, inflammatory arthritis, or immune-mediated arthritides.

30. The pharmaceutical composition of claim 1 or 2, wherein the disorder is an autoimmune or inflammatory disorder.

31. The pharmaceutical composition of claim 29, wherein the autoimmune or inflammatory disorder is selected from the group consisting of an Allergy, Asthma, Multiple sclerosis, Psoriasis, Inflammatory bowel disease, Autoimmune experimental uveitis, Colitis, Hypersensitivity reaction disease, Diabetes, Crohn's disease, Systemic lupus erythematosus, Pulmonary sarcoidosis, Leishmaniasis, Experimental autoimmune encephalomyelitis, HTLV-1-associated myelopathy, Tropical spastic paraparesis, Scleroderma, an Idiopathic inflammatory myopathy, polymyositis, and dermatomyositis.

32. The pharmaceutical composition of any one of claims 1-31, wherein the RNA-guided nuclease or a nucleic acid encoding an RNA-guided nuclease is the RNA-guided nuclease.

33. The pharmaceutical composition of any one of claims 1-31, wherein the RNA-guided nuclease or a nucleic acid encoding an RNA-guided nuclease is DNA encoding the RNA-guided nuclease.

34. The pharmaceutical composition of any one of claims 1-31, wherein the RNA-guided nuclease or a nucleic acid encoding an RNA-guided nuclease is mRNA encoding the RNA-guided nuclease.

35. The pharmaceutical composition of any one of claims 1-34, wherein the RNA-guided nuclease is a Cas protein.

36. The pharmaceutical composition of claim 35, wherein the Cas protein is a Cas9 protein.

37. The pharmaceutical composition of claim 35, wherein the Cas9 protein is an S. pyogenes Cas9 polypeptide.

38. The pharmaceutical composition of claim 35, wherein the Cas9 protein is selected from the group consisting of esCas9, hfCas9, peCas9, and ARCas9.

39. The pharmaceutical composition of any one of claims 1-38, wherein the at least one guide RNA or a nucleic acid encoding at least one guide RNA is the at least one guide RNA.

40. The pharmaceutical composition of any one of claims 1-38, wherein the at least one guide RNA or a nucleic acid encoding at least one guide RNA is DNA encoding the at least one guide RNA.

41. The pharmaceutical composition of any one of claims 1-38, comprising a nucleic acid encoding both the RNA-guided nuclease and the at least one guide RNA.

42. The pharmaceutical composition of any one of claims 1-41, wherein the at least one guide RNA is a single guide RNA (sgRNA).

43. The pharmaceutical composition of any one of claims 1-42, wherein the at least one guide RNA targets a human gene.

44. The pharmaceutical composition of any one of claims 1-42, wherein the at least one guide RNA targets a canine gene.

45. The pharmaceutical composition of any one of claims 1-42, wherein the at least one guide RNA targets an equine gene.

46. The pharmaceutical composition of any one of claims 1-42, wherein the at least one guide RNA targets a feline gene.

47. The pharmaceutical composition of any one of claims 1-42, wherein the at least one guide RNA targets a mammalian gene.

48. The pharmaceutical composition of any one of claims 1-47, wherein the composition is formulated for parenteral administration.

49. The pharmaceutical composition of any one of claims 1-47, wherein the composition is formulated for intra-articular injection within a joint of the subject.

50. The pharmaceutical composition of any one of claims 1-47, wherein the composition is formulated for intradiscal injection.

51. The pharmaceutical composition of any one of claims 1-47, wherein the composition is formulated for peridiscal injection.

52. The pharmaceutical composition of any one of claims 1-47, wherein the composition is formulated for intravertebral injection.

53. The pharmaceutical composition of any one of claims 1-47, wherein the composition is formulated for epidural injection.

54. The pharmaceutical composition of any one of claims 1-47, wherein the composition is formulated for injection to the facet joints of the spine.

55. The pharmaceutical composition of any one of claims 1-54, wherein the one or more LNPs comprise one or more ionizable lipids selected from: 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), 2-({8-[(3.beta.)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2R)), (2S)-2-({8-[(3.beta.)-cholest-5-en-3-yloxy]octyl}oxy)-N,N-dimethyl-3-[(9Z-,12Z)-octadeca-9,12-dien-1-yloxy]propan-1-amine (Octyl-CLinDMA (2S)), LP01, ALC-0315, SM-102, a lipid including a cyclic amine group, and mixtures thereof.

56. The pharmaceutical composition of claim 55, wherein the ionizable lipid is DLin-KC2-DMA.

57. The pharmaceutical composition of claim 55, wherein the ionizable lipid is DLin-MC3-DMA.

58. The pharmaceutical composition of claim 55, wherein the ionizable lipid is SM-102.

59. The pharmaceutical composition of claim 55, wherein the ionizable lipid is ALC-0315.

60. The pharmaceutical composition of claim 55, wherein the ionizable lipid is LP01.

61. The pharmaceutical composition of any one of claims 1 to 54, wherein the one or more LNPs comprise one or more helper lipid selected from: 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-gly cero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-gly cero-3-phospho-rac-(1-glycerol) sodium salt (DOPG), sphingomyelin (SM), and mixtures thereof.

62. The pharmaceutical composition of any one of claims 1 to 54, wherein the one or more LNPs comprise one or more PEG lipids selected from: PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, PEG-DMA or a PEG-DSPE lipid.

63. The pharmaceutical composition of any one of claims 1 to 54, wherein the one or more LNPs comprise one or more structural lipids selected from: cholesterol, fecosterol, stigmasterol, stigmastanol, sitosterol, D-sitosterol, lupeol, betulin, ursolic acid, oleanolic acid, campesterol, fucosterol, brassicasterol, ergosterol, 9, 11-dehydroergosterol, tomatidine, tomatine, α-tocopherol, and mixtures thereof.

64. The pharmaceutical composition of claim 63, wherein the structural lipid is cholesterol.

65. The pharmaceutical composition of claim 63, wherein the one or more LNPs comprise a structural lipid and a corticosteroid (e.g., prednisolone, dexamethasone, prednisone, and hydrocortisone), or a combination thereof.

66. The pharmaceutical composition of claim 65, wherein the corticosteroid is dexamethasone.

67. The pharmaceutical composition of any one of claims 1 to 66, wherein the LNPs comprise a plurality of particles with a diameter over 100 nm.

68. The pharmaceutical composition of any one of claims 1 to 66, wherein the LNPs comprise a plurality of particles with a diameter between about 60 nm and about 120 nm.

69. The pharmaceutical composition of any one of claims 1 to 68, wherein the plurality of LNPs comprise an LNP system selected from any one of LNP001 to LNP240.

70. A method for treating a disease or disorder in a subject in need thereof, the method comprising administering to the subject a pharmaceutical composition of any one of claims 1 to 69.
